(19) United States
(12) Patent Application Publication
(10) Pub. No.: US 2025/0145598 A1
(43) Pub. Date: May 8, 2025

(54) ACID CERAMIDASE INHIBITORS AND USES THEREOF

(71) Applicant: The Regents of the University of California, Oakland, CA (US)

(72) Inventors: Jennifer Y. Chen, San Francisco, CA (US); William F. DeGrado, San Francisco, CA (US); Hyunil Jo, Lafayette, CA (US); Richard Beresis, San Francisco, CA (US); Marc Adler, Orinda, CA (US)

(21) Appl. No.: 18/684,143

(22) PCT Filed: Aug. 17, 2022
(86) PCT No.: PCT/US22/40601

§ 371 Date: Feb. 15, 2024

Related U.S. Application Data
(60) Provisional application No. 63/234,560, filed on Aug. 18, 2021.

(51) Int. Cl. C07D 413/12; A61K 31/4178; A61K 31/4192; A61K 31/4196; A61K 31/4245; A61K 31/427; A61K 31/4439; A61K 31/496; A61K 31/501; A61K 31/506; A61K 31/5377; C07D 233/64; C07D 401/12; C07D 401/14; C07D 403/12; C07D 403/14; C07D 405/14; C07D 413/14; C07D 417/14

(52) U.S. Cl. CPC C07D 413/12; A61K 31/4178; A61K 31/4192; A61K 31/4196; A61K 31/4245; A61K 31/427; A61K 31/4439; A61K 31/496; A61K 31/501; A61K 31/506; A61K 31/5377; C07D 233/64; C07D 401/12; C07D 401/14; C07D 403/12; C07D 403/14; C07D 405/14; C07D 413/14; C07D 417/14

(57) ABSTRACT

Described herein, inter alia, are acid ceramidase inhibitors and uses thereof.

### CROSS-REFERENCES TO RELATED APPLICATIONS

This application claims the benefit of U.S. Provisional Application No. 63/234,560, filed Aug. 18, 2021, which is incorporated herein by reference in its entirety and for all purposes.

### BACKGROUND

Fibrosis develops in response to chronic injury in nearly all organs and is characterized by progressive matrix stiffening. Tissue fibrosis is associated with high morbidity and mortality. Treatment options for fibrosis are limited, and organ transplantation is the only effective option for end-stage disease. Disclosed herein, inter alia, are solutions to these and other problems in the art.

### BRIEF SUMMARY

In an aspect is provided a compound, or a pharmaceutically acceptable salt thereof, having the formula:

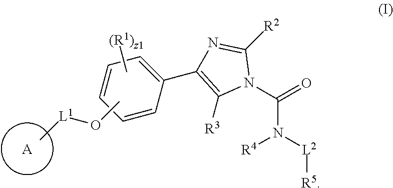

L¹ is a bond, —C(O)—, substituted or unsubstituted alkylene, or substituted or unsubstituted heteroalkylene.

Ring A is substituted or unsubstituted cycloalkyl, substituted or unsubstituted heterocycloalkyl, substituted or unsubstituted aryl, or substituted or unsubstituted heteroaryl.

R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR_{1A}R^{1B}, —NHC(O)NR^{1A}R^{1B}, —N(O)ₘ₁, —NR^{1A}R^{1B}, —C(O)R^{1C}, —C(O)OR^{1C}, —C(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —NR^{1A}OR_{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R¹ substituents may optionally be joined to form a substituted or unsubstituted cycloalkyl, substituted or unsubstituted heterocycloalkyl, substituted or unsubstituted aryl, or substituted or unsubstituted heteroaryl.

The symbol z1 is an integer from 0 to 4.

R² is hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —N^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R³ is hydrogen, halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NR^{3C}NR^{3A}R^{3B}, —ONR^{3A}R^{3B}, —NHC(O)NR^{3C}NR^{3A}R^{3B}, —NHC(O)NR^{3A}R^{3B}, —N(O)ₘ₃, —NR^{3A}R^{3B}, —C(O)R^{3C}, —C(O)OR^{3C}, —C(O)NR^{3A}R^{3B}, —OR^{3D}, —SR^{3D}, —NR^{3A}SO₂R^{3D}, —NR^{3A}C(O)R^{3C}, —NR^{3A}C(O)OR^{3C}, —NR^{3A}OR^{3C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R⁴ is hydrogen or unsubstituted C₁-C₄ alkyl.

L² is -L^{2A}-L^{2B}-L^{2C}-.

L^{2A}, L^{2B}, and L^{2C} are independently a bond, —O—, —NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

R⁵ is hydrogen, halogen, —CX⁵₃, —CHX⁵₂, —CH₂X⁵, —OCX⁵₃, —OCH₂X⁵, —OCHX⁵₂, —CN, —SOₙ₅R^{5D}, —SOᵥ₅NR^{5A}R^{5B}, —NR^{5C}NR^{5A}R^{5B}, —ONR^{5A}R^{5B}, —NHC(O)NR^{5C}NR^{5A}R^{5B}, —NHC(O)NR^{5A}R^{5B}, —N(O)ₘ₅, —NR^{5A}R^{5B}, —C(O)R^{5C}, —C(O)OR^{5C}, —C(O)NR^{5A}R^{5B}, —OR^{5D}, —SR^{5D}, —NR^{5A}SO₂R^{5D}, —NR^{5A}C(O)R^{5C}, —NR^{5A}C(O)OR^{5C}, —NR^{5A}OR^{5C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R^{1A}, R^{1B}, R^{1C}, R^{1D}, R^{2A}, R^{2B}, R^{2C}, R^{2D}, R^{3A}, R^{3B}, R^{3C}, R^{3D}, R^{5A}, R^{5B}, R^{5C}, and R^{5D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{5A} and R^{5B} substituents bonded to the same nitrogen atom may optionally be joined to form a substituted or unsubstituted heterocycloalkyl or substituted or unsubstituted heteroaryl.

X¹, X², X³, and X⁵ are independently —F, —Cl, —Br, or —I.

The symbols n1, n2, n3, and n5 are independently an integer from 0 to 4.

The symbols m1, m2, m3, m5, v1, v2, v3, and v5 are independently 1 or 2.

In an aspect is provided a pharmaceutical composition including a compound described herein, or a pharmaceutically acceptable salt thereof, and a pharmaceutically acceptable excipient.

In an aspect is provided a method of treating fibrotic disease in a subject in need thereof, the method including administering to the subject in need thereof a therapeutically effective amount of an acid ceramidase inhibitor, or a pharmaceutically acceptable salt thereof.

In an aspect is provided a method of treating fibrotic disease in a subject in need thereof, the method including administering to the subject in need thereof a therapeutically effective amount of a compound described herein, or a pharmaceutically acceptable salt thereof.

In an aspect is provided a method of treating cancer in a subject in need thereof, the method including administering to the subject in need thereof a therapeutically effective amount of a compound described herein, or a pharmaceutically acceptable salt thereof.

### DETAILED DESCRIPTION

### I. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di-, and multivalent radicals. The alkyl may include a designated number of carbons (e.g., C₁-C₁₀ means one to ten carbons). In embodiments, the alkyl is fully saturated. In embodiments, the alkyl is monounsaturated. In embodiments, the alkyl is polyunsaturated. Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkenyl includes one or more double bonds. An alkynyl includes one or more triple bonds.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene. The term “alkynylene” by itself or as part of another substituent, means, unless otherwise stated, a divalent radical derived from an alkyne. In embodiments, the alkylene is fully saturated. In embodiments, the alkylene is monounsaturated. In embodiments, the alkylene is polyunsaturated. An alkenylene includes one or more double bonds. An alkynylene includes one or more triple bonds.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —S—CH₂—CH₂, —S(O)—CH₃, —CH₂—CH₂—S(O)₂—CH₃, —CH═CHO—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds. In embodiments, the heteroalkyl is fully saturated. In embodiments, the heteroalkyl is monounsaturated. In embodiments, the heteroalkyl is polyunsaturated.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′ and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like. The term “heteroalkenylene,” by itself or as part of another substituent, means, unless otherwise stated, a divalent radical derived from a heteroalkene. The term “heteroalkynylene” by itself or as part of another substituent, means, unless otherwise stated, a divalent radical derived from a heteroalkyne. In embodiments, the heteroalkylene is fully saturated. In embodiments, the heteroalkylene is monounsaturated. In embodiments, the heteroalkylene is polyunsaturated. A heteroalkenylene includes one or more double bonds. A heteroalkynylene includes one or more triple bonds.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively. In embodiments, the cycloalkyl is fully saturated. In embodiments, the cycloalkyl is monounsaturated. In embodiments, the cycloalkyl is polyunsaturated. In embodiments, the heterocycloalkyl is fully saturated. In embodiments, the heterocycloalkyl is monounsaturated. In embodiments, the heterocycloalkyl is polyunsaturated.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. A bicyclic or multicyclic cycloalkyl ring system refers to multiple rings fused together wherein at least one of the fused rings is a cycloalkyl ring and wherein the multiple rings are attached to the parent molecular moiety through any carbon atom contained within a cycloalkyl ring of the multiple rings.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. A bicyclic or multicyclic cycloalkenyl ring system refers to multiple rings fused together wherein at least one of the fused rings is a cycloalkenyl ring and wherein the multiple rings are attached to the parent molecular moiety through any carbon atom contained within a cycloalkenyl ring of the multiple rings.

In embodiments, the term “heterocycloalkyl” means a monocyclic, bicyclic, or a multicyclic heterocycloalkyl ring system. In embodiments, heterocycloalkyl groups are fully saturated. A bicyclic or multicyclic heterocycloalkyl ring system refers to multiple rings fused together wherein at least one of the fused rings is a heterocycloalkyl ring and wherein the multiple rings are attached to the parent molecular moiety through any atom contained within a heterocycloalkyl ring of the multiple rings.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring and wherein the multiple rings are attached to the parent molecular moiety through any carbon atom contained within an aryl ring of the multiple rings. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring and wherein the multiple rings are attached to the parent molecular moiety through any atom contained within a heteroaromatic ring of the multiple rings). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g., substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g., all rings being substituted heterocycloalkylene wherein each ring may be the same or different substituted heterocycloalkylene). When referring to a spirocyclic ring system, heterocyclic spirocyclic rings means spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

Bridged rings are two or more rings that share three or more atoms, separating the two bridgehead atoms by a bridge containing at least one atom. Individual rings in bridged rings may be substituted or unsubstituted and may have different substituents from other individual rings within a set of bridged rings. Possible substituents for individual rings within bridged rings are the possible substituents for the same ring when not part of bridged rings (e.g., substituents for cycloalkyl or heterocycloalkyl rings). Bridged rings may be substituted or unsubstituted cycloalkyl, substituted or unsubstituted cycloalkylene, substituted or unsubstituted heterocycloalkyl or substituted or unsubstituted heterocycloalkylene and individual rings within a bridged ring group may be any of the immediately previous list, including having all rings of one type (e.g., all rings being substituted heterocycloalkylene wherein each ring may be the same or different substituted heterocycloalkylene). When referring to a bridged ring system, heterocyclic bridged rings means bridged rings wherein at least one ring is a heterocyclic ring and wherein each ring may be a different ring. When referring to a bridged ring system, substituted bridged rings means that at least one ring is substituted and each substituent may optionally be different.

The symbol “ [symbol] ” denotes the point of attachment of a chemical moiety to the remainder of a molecule or chemical formula.

The term “oxo,” as used herein, means an oxygen that is double bonded to a carbon atom.

The term “alkylarylene” as an arylene moiety covalently bonded to an alkylene moiety (also referred to herein as an alkylene linker). In embodiments, the alkylarylene group has the formula:

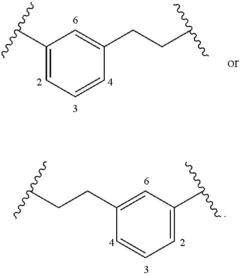 [SMILES: *CCc1cccc(*)c1 ; *CCc1cccc(*)c1]

An alkylarylene moiety may be substituted (e.g., with a substituent group) on the alkylene moiety or the arylene linker (e.g., at carbons 2, 3, 4, or 6) with halogen, oxo, —N₃, —CF₃, —CCl₃, —CBr₃, —CI₃, —CN, —CHO, —OH, —NH₂, —COOH, —CONH₂, —NO₂, —SH, —SO₂CH₃, —SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl). In embodiments, the alkylarylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, halogen, —SiR′R″R′″, —OC(O)R′, —C(O)R′, —CO₂R′, —CONR′R″, —OC(O)NR′R″, —NR″C(O)R′, —NR′C(O)NR″R′″, —NR″C(O)₂R′, —NRC(NR′R″R′″)═NR″″, —NRC(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —NR′SO₂R″, —NR′C(O)R″, —NR′C(O)OR″, —NR′OR″, in a number ranging from zero to (2m′+1), where m′ is the total number of carbon atoms in such radical. R, R′, R′, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R′ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, halogen, —SiR′R″R′″, —OC(O)R′, —C(O)R′, —CO₂R′, —CONR′R″, —OC(O)NR′R″, —NR″C(O)R′, —NR′C(O)NR″R′″, —NR″C(O)₂R′, —NR—C(NR′R″R′″)═NR″″, —NR—C(NR′R″)=NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Substituents for rings (e.g., cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g., a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), selenium (Se), and silicon (Si). In embodiments, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl (e.g., C₁-C₅ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
   (i) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
   (ii) alkyl (e.g., C₁-C₅ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
      (a) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
      (b) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from: oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OC₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted phenyl, and each substituted or unsubstituted heteroaryl is a substituted or unsubstituted 5 to 6 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₅ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, FIGURES, or tables below.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

In a recited claim or chemical formula description herein, each R substituent or L linker that is described as being “substituted” without reference as to the identity of any chemical moiety that composes the “substituted” group (also referred to herein as an “open substitution” on an R substituent or L linker or an “openly substituted” R substituent or L linker), the recited R substituent or L linker may, in embodiments, be substituted with one or more first substituent groups as defined below.

The first substituent group is denoted with a corresponding first decimal point numbering system such that, for example, R¹ may be substituted with one or more first substituent groups denoted by R^{1.1}, R² may be substituted with one or more first substituent groups denoted by R^{2.1}, R³ may be substituted with one or more first substituent groups denoted by R^{3.1}, R⁴ may be substituted with one or more first substituent groups denoted by R⁴-1, R⁵ may be substituted with one or more first substituent groups denoted by R^{5.1}, and the like up to or exceeding an R¹⁰⁰ that may be substituted with one or more first substituent groups denoted by R^{100.1}. As a further example, R^{1A} may be substituted with one or more first substituent groups denoted by R^{1A.1}, R^{2A} may be substituted with one or more first substituent groups denoted by R^{2A.1}, R^{3A} may be substituted with one or more first substituent groups denoted by R^{3A.1}, R^{4A} may be substituted with one or more first substituent groups denoted by R^{4A.1}, R^{5A} may be substituted with one or more first substituent groups denoted by R^{5A.1} and the like up to or exceeding an R^{100A} may be substituted with one or more first substituent groups denoted by R^{100A.1} As a further example, L¹ may be substituted with one or more first substituent groups denoted by R^{L1.1}, L² may be substituted with one or more first substituent groups denoted by R^{L2.1}, L³ may be substituted with one or more first substituent groups denoted by R^{L3.1}, L⁴ may be substituted with one or more first substituent groups denoted by R^{L4.1}, L⁵ may be substituted with one or more first substituent groups denoted by R^{L5.1} and the like up to or exceeding an L¹⁰⁰ which may be substituted with one or more first substituent groups denoted by R^{L100.1}. Thus, each numbered R group or L group (alternatively referred to herein as R^{WW} or L^{WW} wherein “WW” represents the stated superscript number of the subject R group or L group) described herein may be substituted with one or more first substituent groups referred to herein generally as R^{WW.1} or R^{LWW.1}, respectively. In turn, each first substituent group (e.g., R^{1.1}, R^{2.1}, R^{3.1}, R^{4.1}, R^{5.1} . . . R^{100.1}; R^{1A.1}, R^{2A.1}, R^{3A.1}, R^{4A.1}, R^{5A.1} . . . R^{100A.1}; R^{L1.1}, R^{L2.1}, R^{L3.1}, R^{L4.1}, R^{L5.1} . . . R^{L100.1}) may be further substituted with one or more second substituent groups (e.g., R^{1.2}, R^{2.2}, R^{3.2}, R^{4.2}, R^{5.2} . . . R^{100.2}; R^{1A.2}, R^{2A.2}, R^{3A.2}, R^{4A.2}, R^{5A.2} . . . R^{100A.2}; R^{L1.2}, R^{L2.2}, R^{L3.2}, R^{L4.2}, R^{L5.2} . . . R^{L100.2}, respectively). Thus, each first substituent group, which may alternatively be represented herein as R^{WW.1} as described above, may be further substituted with one or more second substituent groups, which may alternatively be represented herein as R^{WW.2}.

Finally, each second substituent group (e.g., R^{1.2}, R^{2.2}, R^{3.2}, R^{4.2}, R^{5.2} . . . R^{100.2}; R^{1A.2}, R^{2A.2}, R^{3A.2}, R^{4A.2}, R^{5A.2} . . . R^{100A.2}; R^{L1.2}, R^{L2.2}, R^{L3.2}, R^{L4.2}, R^{L5.2} . . . R^{L100.2}) may be further substituted with one or more third substituent groups (e.g., R^{1.3}, R^{2.3}, R^{3.3}, R^{4.3}, R^{5.3} . . . R^{100.3}; R^{1A.3}, R^{2A.3}, R^{3A.3}, R^{4A.3}, R^{5A.3} . . . R^{100A.3}; R^{L1.3}, R^{L2.3}, R^{L3.3}, R^{L4.3}, R^{L5.3} . . . R^{L100.3}; respectively). Thus, each second substituent group, which may alternatively be represented herein as R^{WW.2} as described above, may be further substituted with one or more third substituent groups, which may alternatively be represented herein as R^{WW.3}. Each of the first substituent groups may be optionally different. Each of the second substituent groups may be optionally different. Each of the third substituent groups may be optionally different.

Thus, as used herein, R^{WW} represents a substituent recited in a claim or chemical formula description herein which is openly substituted. “WW” represents the stated superscript number of the subject R group (1, 2, 3, 1A, 2A, 3A, 1B, 2B, 3B, etc.). Likewise, L^{WW} is a linker recited in a claim or chemical formula description herein which is openly substituted. Again, “WW” represents the stated superscript number of the subject L group (1, 2, 3, 1A, 2A, 3A, 1B, 2B, 3B, etc.). As stated above, in embodiments, each R^{WW} may be unsubstituted or independently substituted with one or more first substituent groups, referred to herein as R^{WW.1}; each first substituent group, R^{WW.1}, may be unsubstituted or independently substituted with one or more second substituent groups, referred to herein as R^{WW.2}; and each second substituent group may be unsubstituted or independently substituted with one or more third substituent groups, referred to herein as R^{WW.3}. Similarly, each L^{WW} linker may be unsubstituted or independently substituted with one or more first substituent groups, referred to herein as R^{LWW.1}; each first substituent group, R^{LWW.1}, may be unsubstituted or independently substituted with one or more second substituent groups, referred to herein as R^{LWW.2}; and each second substituent group may be unsubstituted or independently substituted with one or more third substituent groups, referred to herein as R^{LWW.3}. Each first substituent group is optionally different. Each second substituent group is optionally different. Each third substituent group is optionally different. For example, if R^{WW} is phenyl, the said phenyl group is optionally substituted by one or more R^{WW.1} groups as defined herein below, e.g., when R^{WW.1} is R^{WW.2}-substituted or unsubstituted alkyl, examples of groups so formed include but are not limited to itself optionally substituted by 1 or more R^{WW.2}, which R^{WW.2} is optionally substituted by one or more R^{WW.3}. By way of example when the R^{WW} group is phenyl substituted by R^{WW.1}, which is methyl, the methyl group may be further substituted to form groups including but not limited to:

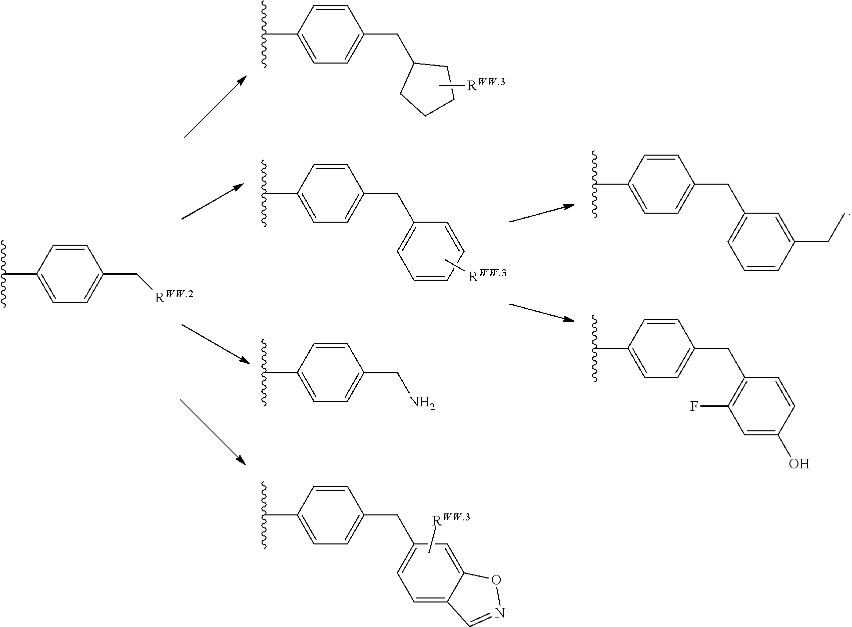

R^{WW.1} is independently oxo, halogen, —CX^{WW.1}₃, —CHX^{WW.1}₂, —CH₂X^{WW.1}, —OCX^{WW.1}₃, —OCH₂X^{WW.1}, —OCHX^{WW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{WW.2}-substituted or unsubstituted alkyl (e.g., C₁-C₈, C₁-C₆, C₁-C₄, or C₁-C₂), R^{WW.2}-substituted or unsubstituted heteroalkyl (e.g., 2 to 8 membered, 2 to 6 membered, 4 to 6 membered, 2 to 3 membered, or 4 to 5 membered), R^{WW.2}-substituted or unsubstituted cycloalkyl (e.g., C₃-C₈, C₃-C₆, C₄-C₆, or C₅-C₆), R^{WW.2}-substituted or unsubstituted heterocycloalkyl (e.g., 3 to 8 membered, 3 to 6 membered, 4 to 6 membered, 4 to 5 membered, or 5 to 6 membered), R^{WW.2}-substituted or unsubstituted aryl (e.g., C₆-C₁₂, C₆-C₁₀, or phenyl), or R^{WW.2}-substituted or unsubstituted heteroaryl (e.g., 5 to 12 membered, 5 to 10 membered, 5 to 9 membered, or 5 to 6 membered). In embodiments, R^{WW.1} is independently oxo, halogen, —CX^{WW.1}₃, —CHX^{WW.1}₂, —CH₂X^{WW.1}, —OCX^{WW.1}₃, —OCH₂X^{WW.1}, —OCHX^{WW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.1} is independently —F, —Cl, —Br, or —I.

R^{WW.2} is independently oxo, halogen, —CX^{WW.2}₃, —CHX^{WW.2}₂, —CH₂X^{WW.2}, —OCX^{WW.2}₃, —OCH₂X^{WW.2}, —OCHX^{WW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{WW.3}-substituted or unsubstituted alkyl (e.g., C₁-C₈, C₁-C₆, C₁-C₄, or C₁-C₂), R^{WW.3}-substituted or unsubstituted heteroalkyl (e.g., 2 to 8 membered, 2 to 6 membered, 4 to 6 membered, 2 to 3 membered, or 4 to 5 membered), R^{WW.3}-substituted or unsubstituted cycloalkyl (e.g., C₃-C₈, C₃-C₆, C₄-C₆, or C₅-C₆), R^{WW.3}-substituted or unsubstituted heterocycloalkyl (e.g., 3 to 8 membered, 3 to 6 membered, 4 to 6 membered, 4 to 5 membered, or 5 to 6 membered), R^{WW.3}-substituted or unsubstituted aryl (e.g., C₆-C₁₂, C₆-C₁₀, or phenyl), or R^{WW.3}-substituted or unsubstituted heteroaryl (e.g., 5 to 12 membered, 5 to 10 membered, 5 to 9 membered, or 5 to 6 membered). In embodiments, R^{WW.2} is independently oxo, halogen, —CX^{WW.2}₃, —CHX^{WW.2}₂, —CH₂X^{WW.2}, —OCX^{WW.2}₃, —OCH₂X^{WW.2}, —OCHX^{WW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.2} is independently —F, —Cl, —Br, or —I.

R^{WW.3} is independently oxo, halogen, —CX^{WW.3}₃, —CHX^{WW.3}₂, —CH₂X^{WW.3}, —OCX^{WW.3}₃, —OCH₂X^{WW.3}, —OCHX^{WW.3}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.3} is independently —F, —Cl, —Br, or —I.

Where two different R^{WW} substituents are joined together to form an openly substituted ring (e.g., substituted cycloalkyl, substituted heterocycloalkyl, substituted aryl or substituted heteroaryl), in embodiments the openly substituted ring may be independently substituted with one or more first substituent groups, referred to herein as R^{WW.1}; each first substituent group, R^{WW.1}, may be unsubstituted or independently substituted with one or more second substituent groups, referred to herein as R^{WW.2}; and each second substituent group, R^{WW.2}, may be unsubstituted or independently substituted with one or more third substituent groups, referred to herein as R^{WW.3}; and each third substituent group, R^{WW.3}, is unsubstituted. Each first substituent group is optionally different. Each second substituent group is optionally different. Each third substituent group is optionally different. In the context of two different R^{WW} substituents joined together to form an openly substituted ring, the “WW” symbol in the R^{WW.1}, R^{WW.2} and R^{WW.3} refers to the designated number of one of the two different R^{WW} substituents. For example, in embodiments where R^{100A} and R^{100B} are optionally joined together to form an openly substituted ring, R^{WW.1} is R^{100A.1}, R^{WW.2} is R^{10A.2}, and R^{WW.3} is R^{100A.3}. Alternatively, in embodiments where R^{100A} and R^{100B} are optionally joined together to form an openly substituted ring, R^{WW.1} is R^{100B.1}, R^{WW.2} is R^{100B.2}, and R^{WW.3} is R^{100B.3}. R^{WW.1}, R^{WW.2} and R^{WW.3} in this paragraph are as defined in the preceding paragraphs.

R^{LWW.1} is independently oxo, halogen, —CX^{LWW.1}₃, —CHX^{LWW.1}₂, —CH₂X^{LWW.1}, —OCX^{LWW.1}₃, —OCH₂X^{LWW.1}, —OCHX^{LWW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{LWW.2}-substituted or unsubstituted alkyl (e.g., C₁-C₈, C₁-C₆, C₁-C₄, or C₁-C₂), R^{LWW.2}-substituted or unsubstituted heteroalkyl (e.g., 2 to 8 membered, 2 to 6 membered, 4 to 6 membered, 2 to 3 membered, or 4 to 5 membered), R^{LWW.2}-substituted or unsubstituted cycloalkyl (e.g., C₃-C₈, C₃-C₆, C₄-C₆, or C₅-C₆), R^{LWW.2}-substituted or unsubstituted heterocycloalkyl (e.g., 3 to 8 membered, 3 to 6 membered, 4 to 6 membered, 4 to 5 membered, or 5 to 6 membered), R^{LWW.2}-substituted or unsubstituted aryl (e.g., C₆-C₁₂, C₆-C₁₀, or phenyl), or R^{LWW.2}-substituted or unsubstituted heteroaryl (e.g., 5 to 12 membered, 5 to 10 membered, 5 to 9 membered, or 5 to 6 membered). In embodiments, R^{LWW.1} is independently oxo, halogen, —CX^{LWW.1}₃, —CHX^{LWW.1}₂, —CH₂X^{LWW.1}, —OCX^{LWW.1}₃, —OCH₂X^{LWW.1}, —OCHX^{LWW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.1} is independently —F, —Cl, —Br, or —I.

R^{LWW.2} is independently oxo, halogen, —CX^{LWW.2}₃, —CHX^{LWW.2}₂, —CH₂X^{LWW.2}, —OCX^{LWW.2}₃, —OCH₂X^{LWW.2}, —OCHX^{LWW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{LWW.3}-substituted or unsubstituted alkyl (e.g., C₁-C₈, C₁-C₆, C₁-C₄, or C₁-C₂), R^{LWW.3}-substituted or unsubstituted heteroalkyl (e.g., 2 to 8 membered, 2 to 6 membered, 4 to 6 membered, 2 to 3 membered, or 4 to 5 membered), R^{WW.3}-substituted or unsubstituted cycloalkyl (e.g., C₃-C₈, C₃-C₆, C₄-C₆, or C₅-C₆), R^{LWW.3}-substituted or unsubstituted heterocycloalkyl (e.g., 3 to 8 membered, 3 to 6 membered, 4 to 6 membered, 4 to 5 membered, or 5 to 6 membered), R^{LWW.3}-substituted or unsubstituted aryl (e.g., C₆-C₁₂, C₆-C₁₀, or phenyl), or R^{LWW.3}-substituted or unsubstituted heteroaryl (e.g., 5 to 12 membered, 5 to 10 membered, 5 to 9 membered, or 5 to 6 membered). In embodiments, R^{LWW.2} is independently oxo, halogen, —CX^{LWW.2}₃, —CHX^{LWW.2}₂, —CH₂X^{LWW.2}, —OCX^{LWW.2}₃, —OCH₂X^{LWW.2}, —OCHX^{LWW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.2} is independently —F, —Cl, —Br, or —I.

R^{LWW.3} is independently oxo, halogen, —CX^{LWW.3}₃, —CHX^{LWW.3}₂, —CH₂X^{LWW.3}, —OCX^{LWW.3}₃, —OCH₂X^{LWW.3}, —OCHX^{LWW.3}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.3} is independently —F, —Cl, —Br, or —I.

In the event that any R group recited in a claim or chemical formula description set forth herein (R^{WW} substituent) is not specifically defined in this disclosure, then that R group (R^{WW} group) is hereby defined as independently oxo, halogen, —CX^{WW}₃, —CHX^{WW}₂, —CH₂X^{WW}, —OCX^{WW}₃, —OCH₂X^{WW}, —OCHX^{WW}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{WW.1}-substituted or unsubstituted alkyl (e.g., C₁-C₈, C₁-C₆, C₁-C₄, or C₁-C₂), R^{WW.1}-substituted or unsubstituted heteroalkyl (e.g., 2 to 8 membered, 2 to 6 membered, 4 to 6 membered, 2 to 3 membered, or 4 to 5 membered), R^{WW.1}-substituted or unsubstituted cycloalkyl (e.g., C₃-C₈, C₃-C₆, C₄-C₆, or C₅-C₆), R^{WW.1}-substituted or unsubstituted heterocycloalkyl (e.g., 3 to 8 membered, 3 to 6 membered, 4 to 6 membered, 4 to 5 membered, or 5 to 6 membered), R^{WW.1}-substituted or unsubstituted aryl (e.g., C₆-C₁₂, C₆-C₁₀, or phenyl), or R^{WW.1}-substituted or unsubstituted heteroaryl (e.g., 5 to 12 membered, 5 to 10 membered, 5 to 9 membered, or 5 to 6 membered). X^{WW} is independently —F, —Cl, —Br, or —I. Again, “WW” represents the stated superscript number of the subject R group (e.g., 1, 2, 3, 1A, 2A, 3A, 1B, 2B, 3B, etc.). R^{WW.1}, R^{WW.2}, and R^{WW.3} are as defined above.

In the event that any L linker group recited in a claim or chemical formula description set forth herein (i.e., an L^{WW} substituent) is not explicitly defined, then that L group (L^{WW} group) is herein defined as independently a bond, —O—, —NH—, —C(O)—, —C(O)NH—, —NHC(O)—, —NHC(O)NH—, —NHC(NH)NH—, —C(O)O—, —OC(O)—, —S—, —SO₂—, —SO₂NH—, R^{LWW.1}-substituted or unsubstituted alkylene (e.g., C₁-C₈, C₁-C₆, C₁-C₄, or C₁-C₂), R^{LWW.1}-substituted or unsubstituted heteroalkylene (e.g., 2 to 8 membered, 2 to 6 membered, 4 to 6 membered, 2 to 3 membered, or 4 to 5 membered), R^{LWW.1}-substituted or unsubstituted cycloalkylene (e.g., C₃-C₈, C₃-C₆, C₄-C₆, or C₅-C₆), R^{LWW.1}-substituted or unsubstituted heterocycloalkylene (e.g., 3 to 8 membered, 3 to 6 membered, 4 to 6 membered, 4 to 5 membered, or 5 to 6 membered), R^{LWW.1}-substituted or unsubstituted arylene (e.g., C₆-C₁₂, C₆-C₁₀, or phenyl), or R^{LWW.1}-substituted or unsubstituted heteroarylene (e.g., 5 to 12 membered, 5 to 10 membered, 5 to 9 membered, or 5 to 6 membered). Again, “WW” represents the stated superscript number of the subject L group (1, 2, 3, 1A, 2A, 3A, 1B, 2B, 3B, etc.). R^{LWW.1}, as well as R^{LWW.2} and R^{LWW.3} are as defined above.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)-or (S)— or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present disclosure, whether radioactive or not, are encompassed within the scope of the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

As used herein, the terms “bioconjugate” and “bioconjugate linker” refer to the resulting association between atoms or molecules of bioconjugate reactive groups or bioconjugate reactive moieties. The association can be direct or indirect. For example, a conjugate between a first bioconjugate reactive group (e.g., —NH₂, —COOH, —N-hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g., a first linker of second linker), or indirect, e.g., by non-covalent bond (e.g., electrostatic interactions (e.g., ionic bond, hydrogen bond, halogen bond), van der Waals interactions (e.g., dipole-dipole, dipole-induced dipole, London dispersion), ring stacking (pi effects), hydrophobic interactions and the like). In embodiments, bioconjugates or bioconjugate linkers are formed using bioconjugate chemistry (i.e., the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g., a sulfhydryl). In embodiments, the first bioconjugate reactive group (e.g., haloacetyl moiety) is covalently attached to the second bioconjugate reactive group (e.g., a sulfhydryl). In embodiments, the first bioconjugate reactive group (e.g., pyridyl moiety) is covalently attached to the second bioconjugate reactive group (e.g., a sulfhydryl). In embodiments, the first bioconjugate reactive group (e.g., —N-hydroxysuccinimide moiety) is covalently attached to the second bioconjugate reactive group (e.g., an amine). In embodiments, the first bioconjugate reactive group (e.g., maleimide moiety) is covalently attached to the second bioconjugate reactive group (e.g., a sulfhydryl). In embodiments, the first bioconjugate reactive group (e.g., -sulfo-N-hydroxysuccinimide moiety) is covalently attached to the second bioconjugate reactive group (e.g., an amine).

Useful bioconjugate reactive moieties used for bioconjugate chemistries herein include, for example: (a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters; (b) hydroxyl groups which can be converted to esters, ethers, aldehydes, etc.; (c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom; (d) dienophile groups which are capable of participating in Diels-Alder reactions such as, for example, maleimido or maleimide groups; (e) aldehyde or ketone groups such that subsequent derivatization is possible via formation of carbonyl derivatives such as, for example, imines, hydrazones, semicarbazones or oximes, or via such mechanisms as Grignard addition or alkyllithium addition; (f) sulfonyl halide groups for subsequent reaction with amines, for example, to form sulfonamides; (g) thiol groups, which can be converted to disulfides, reacted with acyl halides, or bonded to metals such as gold, or react with maleimides; (h) amine or sulfhydryl groups (e.g., present in cysteine), which can be, for example, acylated, alkylated or oxidized; (i) alkenes, which can undergo, for example, cycloadditions, acylation, Michael addition, etc.; (j) epoxides, which can react with, for example, amines and hydroxyl compounds; (k) phosphoramidites and other standard functional groups useful in nucleic acid synthesis; (l) metal silicon oxide bonding; (m) metal bonding to reactive phosphorus groups (e.g., phosphines) to form, for example, phosphate diester bonds; (n) azides coupled to alkynes using copper catalyzed cycloaddition click chemistry; and (o) biotin conjugate can react with avidin or streptavidin to form an avidin-biotin complex or streptavidin-biotin complex.

The bioconjugate reactive groups can be chosen such that they do not participate in, or interfere with, the chemical stability of the conjugate described herein. Alternatively, a reactive functional group can be protected from participating in the crosslinking reaction by the presence of a protecting group. In embodiments, the bioconjugate comprises a molecular entity derived from the reaction of an unsaturated bond, such as a maleimide, and a sulfhydryl group.

“Analog,” “analogue,” or “derivative” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an”, as used in herein means one or more. In addition, the phrase “substituted with a[n]”, as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl”, the group may contain one or more unsubstituted C₁-C₂₀ alkyls, and/or one or more unsubstituted 2 to 20 membered heteroalkyls.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R¹³ substituents are present, each R¹³ substituent may be distinguished as R^{13.A}, R^{13.B}, R^{13.C}, R^{13.D}, etc., wherein each of R^{13.A}, R^{13.B}, R^{13.C}, R^{13.D}, etc. is defined within the scope of the definition of R¹³ and optionally differently.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Thus, the compounds of the present disclosure may exist as salts, such as with pharmaceutically acceptable acids. The present disclosure includes such salts. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g., methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents.

In addition to salt forms, the present disclosure provides compounds, which are in a prodrug form. Prodrugs of the compounds described herein are those compounds that readily undergo chemical changes under physiological conditions to provide the compounds of the present disclosure. Prodrugs of the compounds described herein may be converted in vivo after administration. Additionally, prodrugs can be converted to the compounds of the present disclosure by chemical or biochemical methods in an ex vivo environment, such as, for example, when contacted with a suitable enzyme or chemical reagent.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

A polypeptide, or a cell is “recombinant” when it is artificial or engineered, or derived from or contains an artificial or engineered protein or nucleic acid (e.g., non-natural or not wild type). For example, a polynucleotide that is inserted into a vector or any other heterologous location, e.g., in a genome of a recombinant organism, such that it is not associated with nucleotide sequences that normally flank the polynucleotide as it is found in nature is a recombinant polynucleotide. A protein expressed in vitro or in vivo from a recombinant polynucleotide is an example of a recombinant polypeptide. Likewise, a polynucleotide sequence that does not appear in nature, for example a variant of a naturally occurring gene, is recombinant.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaroytic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

The terms “treating” or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. For example, the certain methods presented herein successfully treat cancer by decreasing the incidence of cancer and or causing remission of cancer. In some embodiments of the compositions or methods described herein, treating cancer includes slowing the rate of growth or spread of cancer cells, reducing metastasis, or reducing the growth of metastatic tumors. The term “treating” and conjugations thereof, include prevention of an injury, pathology, condition, or disease. In embodiments, treating is preventing. In embodiments, treating does not include preventing. In embodiments, the treating or treatment is not prophylactic treatment.

An “effective amount” is an amount sufficient for a compound to accomplish a stated purpose relative to the absence of the compound (e.g., achieve the effect for which it is administered, treat a disease, reduce enzyme activity, increase enzyme activity, reduce signaling pathway, reduce one or more symptoms of a disease or condition. An example of an “effective amount” is an amount sufficient to contribute to the treatment, prevention, or reduction of a symptom or symptoms of a disease, which could also be referred to as a “therapeutically effective amount” when referred to in this context. A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. An “activity increasing amount,” as used herein, refers to an amount of agonist required to increase the activity of an enzyme relative to the absence of the agonist. A “function increasing amount,” as used herein, refers to the amount of agonist required to increase the function of an enzyme or protein relative to the absence of the agonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity (e.g., signaling pathway) of a protein in the absence of a compound as described herein (including embodiments, examples, FIGURES, or Tables).

“Contacting” is used in accordance with its plain ordinary meaning and refers to the process of allowing at least two distinct species (e.g., chemical compounds including biomolecules, or cells) to become sufficiently proximal to react, interact or physically touch. It should be appreciated; however, the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be a compound as described herein and a cellular component (e.g., protein, ion, lipid, nucleic acid, nucleotide, amino acid, protein, particle, organelle, cellular compartment, microorganism, virus, lipid droplet, vesicle, small molecule, protein complex, protein aggregate, or macromolecule). In some embodiments contacting includes allowing a compound described herein to interact with a cellular component (e.g., protein, ion, lipid, nucleic acid, nucleotide, amino acid, protein, particle, virus, lipid droplet, organelle, cellular compartment, microorganism, vesicle, small molecule, protein complex, protein aggregate, or macromolecule) that is involved in a signaling pathway.

As defined herein, the term “activation,” “activate,” “activating” and the like in reference to a protein refers to conversion of a protein into a biologically active derivative from an initial inactive or deactivated state. The terms reference activation, or activating, sensitizing, or up-regulating signal transduction or enzymatic activity or the amount of a protein decreased in a disease.

The terms “agonist,” “activator,” “upregulator,” etc. refer to a substance capable of detectably increasing the expression or activity of a given gene or protein. The agonist can increase expression or activity by at least 10%, 20%, 30%, 40%, 50%, 60%, 70%, 80%, 90%, 95%, 96%, 97%, 98%, or 99% in comparison to a control in the absence of the agonist. In certain instances, expression or activity is 1.5-fold, 2-fold, 3-fold, 4-fold, 5-fold, 10-fold or higher than the expression or activity in the absence of the agonist.

As defined herein, the term “inhibition,” “inhibit,” “inhibiting” and the like in reference to a cellular component-inhibitor interaction means negatively affecting (e.g., decreasing) the activity or function of the cellular component (e.g., decreasing the signaling pathway stimulated by a cellular component (e.g., protein, ion, lipid, virus, lipid droplet, nucleic acid, nucleotide, amino acid, protein, particle, organelle, cellular compartment, microorganism, vesicle, small molecule, protein complex, protein aggregate, or macromolecule)), relative to the activity or function of the cellular component in the absence of the inhibitor. In embodiments inhibition means negatively affecting (e.g., decreasing) the concentration or levels of the cellular component relative to the concentration or level of the cellular component in the absence of the inhibitor. In some embodiments, inhibition refers to reduction of a disease or symptoms of disease. In some embodiments, inhibition refers to a reduction in the activity of a signal transduction pathway or signaling pathway (e.g., reduction of a pathway involving the cellular component). Thus, inhibition includes, at least in part, partially or totally blocking stimulation, decreasing, preventing, or delaying activation, or inactivating, desensitizing, or down-regulating the signaling pathway or enzymatic activity or the amount of a cellular component.

The terms “inhibitor,” “repressor,” “antagonist,” or “downregulator” interchangeably refer to a substance capable of detectably decreasing the expression or activity of a given gene or protein. The antagonist can decrease expression or activity by at least 10%, 20%, 30%, 40%, 50%, 60%, 70%, 80%, 90%, 95%, 96%, 97%, 98%, or 99% in comparison to a control in the absence of the antagonist. In certain instances, expression or activity is 1.5-fold, 2-fold, 3-fold, 4-fold, 5-fold, 10-fold or lower than the expression or activity in the absence of the antagonist.

The term “modulator” refers to a composition that increases or decreases the level of a target molecule or the function of a target molecule or the physical state of the target of the molecule (e.g., a target may be a cellular component (e.g., protein, ion, lipid, virus, lipid droplet, nucleic acid, nucleotide, amino acid, protein, particle, organelle, cellular compartment, microorganism, vesicle, small molecule, protein complex, protein aggregate, or macromolecule)) relative to the absence of the composition.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

“Patient”, “patient in need thereof”, “subject”, or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human. In embodiments, a patient in need thereof is human. In embodiments, a subject is human. In embodiments, a subject in need thereof is human.

“Disease” or “condition” refer to a state of being or health status of a patient or subject capable of being treated with the compounds or methods provided herein. In some embodiments, the disease is a disease related to (e.g., caused by) a cellular component (e.g., protein, ion, lipid, nucleic acid, nucleotide, amino acid, protein, particle, organelle, cellular compartment, microorganism, vesicle, small molecule, protein complex, protein aggregate, or macromolecule). In embodiments, the disease is a fibrotic disease (e.g., nonalcoholic steatohepatitis or liver fibrosis). In embodiments, the disease is cancer (e.g., liver cancer).

As used herein, the terms “fibrotic disease” and “fibrosis” refer to any disease or condition characterized by the formation of excess fibrous connective tissue. The formation of excess fibrous connective tissue may be in response to a reparative or reactive process. Fibrotic diseases include but are not limited to pulmonary fibrosis (e.g., idiopathic pulmonary fibrosis (IPF)), liver fibrosis (e.g., nonalcoholic steatohepatitis (NASH)), myelofibrosis, skin fibrosis (e.g., scleroderma), ocular fibrosis, mediastinal fibrosis, cardiac fibrosis, kidney fibrosis, stromal fibrosis, epidural fibrosis, epithelial fibrosis, or idiopathic fibrosis.

As used herein, the term “cancer” refers to all types of cancer, neoplasm or malignant tumors found in mammals (e.g., humans), including leukemia, lymphoma, carcinomas and sarcomas. Exemplary cancers that may be treated with a compound or method provided herein include cancer of the thyroid, endocrine system, brain, breast, cervix, colon, head and neck, liver, kidney, lung, non-small cell lung, melanoma, mesothelioma, ovary, sarcoma, stomach, uterus, medulloblastoma, colorectal cancer, or pancreatic cancer. Additional examples include, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

As used herein, the term “lymphoma” refers to a group of cancers affecting hematopoietic and lymphoid tissues. It begins in lymphocytes, the blood cells that are found primarily in lymph nodes, spleen, thymus, and bone marrow. Two main types of lymphoma are non-Hodgkin lymphoma and Hodgkin's disease. Hodgkin's disease represents approximately 15% of all diagnosed lymphomas. This is a cancer associated with Reed-Sternberg malignant B lymphocytes. Non-Hodgkin's lymphomas (NHL) can be classified based on the rate at which cancer grows and the type of cells involved. There are aggressive (high grade) and indolent (low grade) types of NHL. Based on the type of cells involved, there are B-cell and T-cell NHLs. Exemplary B-cell lymphomas that may be treated with a compound or method provided herein include, but are not limited to, small lymphocytic lymphoma, Mantle cell lymphoma, follicular lymphoma, marginal zone lymphoma, extranodal (MALT) lymphoma, nodal (monocytoid B-cell) lymphoma, splenic lymphoma, diffuse large cell B-lymphoma, Burkitt's lymphoma, lymphoblastic lymphoma, immunoblastic large cell lymphoma, or precursor B-lymphoblastic lymphoma. Exemplary T-cell lymphomas that may be treated with a compound or method provided herein include, but are not limited to, cutaneous T-cell lymphoma, peripheral T-cell lymphoma, anaplastic large cell lymphoma, mycosis fungoides, and precursor T-lymphoblastic lymphoma.

The term “sarcoma” generally refers to a tumor which is made up of a substance like the embryonic connective tissue and is generally composed of closely packed cells embedded in a fibrillar or homogeneous substance. Sarcomas that may be treated with a compound or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

As used herein, the terms “metastasis,” “metastatic,” and “metastatic cancer” can be used interchangeably and refer to the spread of a proliferative disease or disorder, e.g., cancer, from one organ or another non-adjacent organ or body part. “Metastatic cancer” is also called “Stage IV cancer.” Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

The terms “cutaneous metastasis” or “skin metastasis” refer to secondary malignant cell growths in the skin, wherein the malignant cells originate from a primary cancer site (e.g., breast). In cutaneous metastasis, cancerous cells from a primary cancer site may migrate to the skin where they divide and cause lesions. Cutaneous metastasis may result from the migration of cancer cells from breast cancer tumors to the skin.

The term “visceral metastasis” refer to secondary malignant cell growths in the interal organs (e.g., heart, lungs, liver, pancreas, intestines) or body cavities (e.g., pleura, peritoneum), wherein the malignant cells originate from a primary cancer site (e.g., head and neck, liver, breast). In visceral metastasis, cancerous cells from a primary cancer site may migrate to the internal organs where they divide and cause lesions. Visceral metastasis may result from the migration of cancer cells from liver cancer tumors or head and neck tumors to internal organs.

The term “drug” is used in accordance with its common meaning and refers to a substance which has a physiological effect (e.g., beneficial effect, is useful for treating a subject) when introduced into or to a subject (e.g., in or on the body of a subject or patient). A drug moiety is a radical of a drug.

A “detectable agent,” “detectable compound,” “detectable label,” or “detectable moiety” is a substance (e.g., element), molecule, or composition detectable by spectroscopic, photochemical, biochemical, immunochemical, chemical, magnetic resonance imaging, or other physical means. For example, detectable agents include ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹ Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁵Ac, Cr, V, Mn, Fe, Co, Ni, Cu, La, Ce, Pr, Nd, Pm, Sm, Eu, Gd, Tb, Dy, Ho, Er, Tm, Yb, Lu, ³²P, fluorophore (e.g., fluorescent dyes), modified oligonucleotides (e.g., moieties described in PCT/US2015/022063, which is incorporated herein by reference), electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monochrystalline iron oxide nanoparticles, monochrystalline iron oxide, nanoparticle contrast agents, liposomes or other delivery vehicles containing Gadolinium chelate (“Gd-chelate”) molecules, Gadolinium, radioisotopes, radionuclides (e.g., carbon-11, nitrogen-13, oxygen-15, fluorine-18, rubidium-82), fluorodeoxyglucose (e.g., fluorine-18 labeled), any gamma ray emitting radionuclides, positron-emitting radionuclide, radiolabeled glucose, radiolabeled water, radiolabeled ammonia, biocolloids, microbubbles (e.g., including microbubble shells including albumin, galactose, lipid, and/or polymers; microbubble gas core including air, heavy gas(es), perfluorcarbon, nitrogen, octafluoropropane, perflexane lipid microsphere, perflutren, etc.), iodinated contrast agents (e.g., iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide.

Radioactive substances (e.g., radioisotopes) that may be used as imaging and/or labeling agents in accordance with the embodiments of the disclosure include, but are not limited to, ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹n, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra and ²²⁵Ac. Paramagnetic ions that may be used as additional imaging agents in accordance with the embodiments of the disclosure include, but are not limited to, ions of transition and lanthanide metals (e.g., metals having atomic numbers of 21-29, 42, 43, 44, or 57-71). These metals include ions of Cr, V, Mn, Fe, Co, Ni, Cu, La, Ce, Pr, Nd, Pm, Sm, Eu, Gd, Th, Dy, Ho, Er, Tm, Yb, and Lu.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

As used herein, the term “administering” is used in accordance with its plain and ordinary meaning and includes oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds of the invention can be administered alone or can be co-administered to the patient. Co-administration is meant to include simultaneous or sequential administration of the compounds individually or in combination (more than one compound). Thus, the preparations can also be combined, when desired, with other active substances (e.g., to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols.

The compounds described herein can be used in combination with one another, with other active agents known to be useful in treating a disease associated with cells expressing a disease associated cellular component, or with adjunctive agents that may not be effective alone, but may contribute to the efficacy of the active agent.

In some embodiments, co-administration includes administering one active agent within 0.5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents. In other embodiments, the active agents can be formulated separately. In another embodiment, the active and/or adjunctive agents may be linked or conjugated to one another.

In therapeutic use for the treatment of a disease, compound utilized in the pharmaceutical compositions of the present invention may be administered at the initial dosage of about 0.001 mg/kg to about 1000 mg/kg daily. A daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosages, however, may be varied depending upon the requirements of the patient, the severity of the condition being treated, and the compound or drug being employed. For example, dosages can be empirically determined considering the type and stage of disease (e.g., fibrotic disease or cancer) diagnosed in a particular patient. The dose administered to a patient, in the context of the present invention, should be sufficient to affect a beneficial therapeutic response in the patient over time. The size of the dose will also be determined by the existence, nature, and extent of any adverse side effects that accompany the administration of a compound in a particular patient. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. For convenience, the total daily dosage may be divided and administered in portions during the day, if desired.

The term “associated” or “associated with” in the context of a substance or substance activity or function associated with a disease (e.g., a protein associated disease, disease associated with a cellular component) means that the disease (e.g., fibrotic disease or cancer) is caused by (in whole or in part), or a symptom of the disease is caused by (in whole or in part) the substance or substance activity or function or the disease or a symptom of the disease may be treated by modulating (e.g., inhibiting or activating) the substance (e.g., cellular component). As used herein, what is described as being associated with a disease, if a causative agent, could be a target for treatment of the disease.

The term “aberrant” as used herein refers to different from normal. When used to describe enzymatic activity, aberrant refers to activity that is greater or less than a normal control or the average of normal non-diseased control samples. Aberrant activity may refer to an amount of activity that results in a disease, wherein returning the aberrant activity to a normal or non-disease-associated amount (e.g., by administering a compound or using a method as described herein), results in reduction of the disease or one or more disease symptoms.

The term “isolated,” when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide,” and “protein” are used interchangeably herein to refer to a polymer of amino acid residues, wherein the polymer may in embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

The term “protein complex” is used in accordance with its plain ordinary meaning and refers to a protein which is associated with an additional substance (e.g., another protein, protein subunit, or a compound). Protein complexes typically have defined quaternary structure. The association between the protein and the additional substance may be a covalent bond. In embodiments, the association between the protein and the additional substance (e.g., compound) is via non-covalent interactions. In embodiments, a protein complex refers to a group of two or more polypeptide chains. Proteins in a protein complex are linked by non-covalent protein-protein interactions. A non-limiting example of a protein complex is the proteasome.

The term “acid ceramidase” refers to a protein (including homologs, isoforms, and functional fragments thereof) that cleaves fatty acids from ceramide. The term includes any recombinant or naturally-occurring form of acid ceramidase variants thereof that maintain acid ceramidase activity (e.g., within at least 30%, 40%, 50%, 60%, 70%, 80%, 90%, 95%, or 100% activity compared to wildtype acid ceramidase). In embodiments, the acid ceramidase protein encoded by the ASAHI gene has the amino acid sequence set forth in or corresponding to Entrez 427, UniProt Q13510, RefSeq (protein) NP_001120977.1, RefSeq (protein) NP 004306.3, or RefSeq (protein) NP_808592.2. In embodiments, the ASAHI gene has the nucleic acid sequence set forth in RefSeq (mRNA) NM_001127505.2, RefSeq (mRNA) NM 004315.5, or RefSeq (mRNA) NM_177924.4. In embodiments, the amino acid sequence or nucleic acid sequence is the sequence known at the time of filing of the present application.

The term “acid ceramidase inhibitor” as used herein refers to a substance (e.g., a compound described herein) that is capable of decreasing the expression or activity of acid ceramidase compared to the absence of the acid ceramidase inhibitor. In embodiments, the acid ceramidase inhibitor can decrease expression or activity by at least 10%, 20%, 30%, 40%, 50%, 60%, 70%, 80%, 90%, 95%, 96%, 97%, 98%, or 99% in comparison to a control in the absence of the acid ceramidase inhibitor. In certain instances, expression or activity is 1.5-fold, 2-fold, 3-fold, 4-fold, 5-fold, 10-fold or lower than the expression or activity in the absence of the acid ceramidase inhibitor.

### II. Compounds

In an aspect is provided a compound, or a pharmaceutically acceptable salt thereof, having the formula:

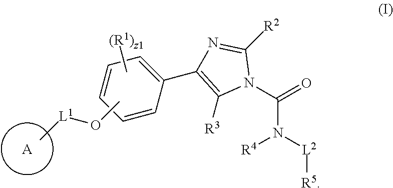

L¹ is a bond, —C(O)—, substituted or unsubstituted alkylene (e.g., C₁-C₈, C₁-C₆, C₁-C₄, or C₁-C₂), or substituted or unsubstituted heteroalkylene (e.g., 2 to 8 membered, 2 to 6 membered, 4 to 6 membered, 2 to 3 membered, or 4 to 5 membered).

Ring A is substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)OR^{1C}, —C(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₃-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); two R¹ substituents may optionally be joined to form a substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

The symbol z1 is an integer from 0 to 4.

R² is hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ON_{2A}R_{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R³ is hydrogen, halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NR^{3C}NR^{3A}R^{3B}, —ONR^{3A}R^{3B}, —NHC(O)NR^{3C}NR^{3A}R^{3B}, —NHC(O)NR^{3A}R^{3B}, —N(O)ₘ₃, —NR^{3A}R^{3B}, —C(O)R^{3C}, —C(O)OR^{3C}, —C(O)NR^{3A}R^{3B}, —OR^{3D}, —SR^{3D}, —NR^{3A}SO₂R^{3D}, —NR^{3A}C(O)R^{3C}, —NR^{3A}C(O)OR^{3C}, —NR^{3A}OR^{3C}, —SF₅, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R⁴ is hydrogen or unsubstituted C₁-C₄ alkyl.

L² is -L^{2A}-L^{2B}-L^{2C}-.

L^{2A}, L^{2B}, and L^{2C} are independently a bond, —O—, —NH—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

R⁵ is hydrogen, halogen, —CX⁵₃, —CHX⁵₂, —CH₂X⁵, —OCX⁵₃, —OCH₂X⁵, —OCHX⁵₂, —CN, —SOₙ₅R^{5D}, —SOᵥ₅NR^{5A}R^{5B}, —NR^{5C}NR^{5A}R^{5B}, —ONR^{5A}R^{5B}, —NHC(O)NR^{5C}NR^{5A}R^{5B}, —NHC(O)NR^{5A}R^{5B}, —N(O)ₘ₅, —NR^{5A}R^{5B}, —C(O)R^{5C}, —C(O)OR^{5C}, —C(O)NR^{5A}R^{5B}, —OR^{5D}, —SR^{5D}, —NR^{5A}SO₂R^{5D}, —NR^{5A}C(O)R^{5C}, —NR^{5A}C(O)OR^{5C}, —NR^{5A}OR^{5C}, —SF₅, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R^{1A}, R^{1B}, R^{1C}, R^{1D}, R^{2A}, R^{2B}, R^{2C}, R^{2D}, R^{3A}, R^{3B}, R^{3C}, R^{3D}, R^{5A}, R^{5B}, R^{5C}, and R^{5D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); R^{1A} and R^{1B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered); R^{2A} and R^{2B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered); R^{3A} and R^{3B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered); R^{5A} and R^{5B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered).

X¹, X², X³, and X⁵ are independently —F, —Cl, —Br, or —I.

The symbols n1, n2, n3, and n5 are independently an integer from 0 to 4.

The symbols m1, m2, m3, m5, v1, v2, v3, and v5 are independently 1 or 2.

In embodiments, the compound has the formula:

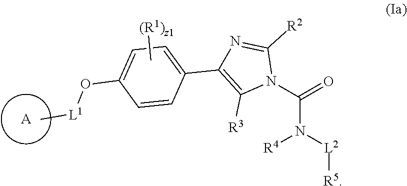

Ring A, R¹, z1, R², R³, R⁴, R⁵, L¹, and L² are as described herein, including in embodiments.

In embodiments, the compound has the formula:

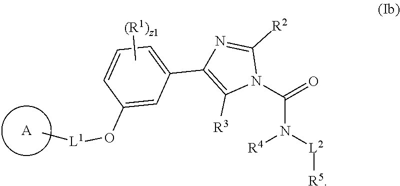

Ring A, R¹, z1, R², R³, R⁴, R⁵, L¹, and L² are as described herein, including in embodiments.

In embodiments, the compound has the formula:

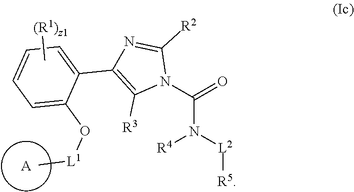

Ring A, R¹, z1, R², R³, R⁴, R⁵, L¹, and L² are as described herein, including in embodiments.

In embodiments, a substituted Ring A (e.g., substituted cycloalkyl, substituted heterocycloalkyl, substituted aryl, and/or substituted heteroaryl) is substituted with at least one substituent group, size-limited substituent group, or lower substituent group; wherein if the substituted Ring A is substituted with a plurality of groups selected from substituent groups, size-limited substituent groups, and lower substituent groups; each substituent group, size-limited substituent group, and/or lower substituent group may optionally be different. In embodiments, when Ring A is substituted, it is substituted with at least one substituent group. In embodiments, when Ring A is substituted, it is substituted with at least one size-limited substituent group. In embodiments, when Ring A is substituted, it is substituted with at least one lower substituent group.

In embodiments, Ring A is substituted or unsubstituted 5 to 9 membered cycloalkyl, substituted or unsubstituted 5 to 6 membered heterocycloalkyl, substituted or unsubstituted phenyl, or substituted or unsubstituted 5 to 9 membered heteroaryl. In embodiments, Ring A is substituted or unsubstituted 5 to 9 membered cycloalkyl. In embodiments, Ring A is substituted or unsubstituted 5 to 6 membered heterocycloalkyl. In embodiments, Ring A is substituted or unsubstituted piperidinyl. In embodiments, Ring A is substituted or unsubstituted piperazinyl. In embodiments, Ring A is substituted or unsubstituted morpholinyl. In embodiments, Ring A is substituted or unsubstituted tetrahydropyranyl. In embodiments, Ring A is substituted or unsubstituted phenyl. In embodiments, Ring A is substituted or unsubstituted 5 to 9 membered heteroaryl. In embodiments, Ring A is substituted or unsubstituted pyridyl. In embodiments, Ring A is substituted or unsubstituted 2-pyridyl. In embodiments, Ring A is substituted or unsubstituted 3-pyridyl. In embodiments, Ring A is substituted or unsubstituted 4-pyridyl. In embodiments, Ring A is substituted or unsubstituted pyrimidinyl. In embodiments, Ring A is substituted or unsubstituted pyridazinyl. In embodiments, Ring A is substituted or unsubstituted oxazolyl. In embodiments, Ring A is substituted or unsubstituted pyrazolyl. In embodiments, Ring A is substituted or unsubstituted triazolyl. In embodiments, Ring A is substituted or unsubstituted oxadiazolyl. In embodiments, Ring A is substituted or unsubstituted thiazolyl.

In embodiments, the compound has the formula:

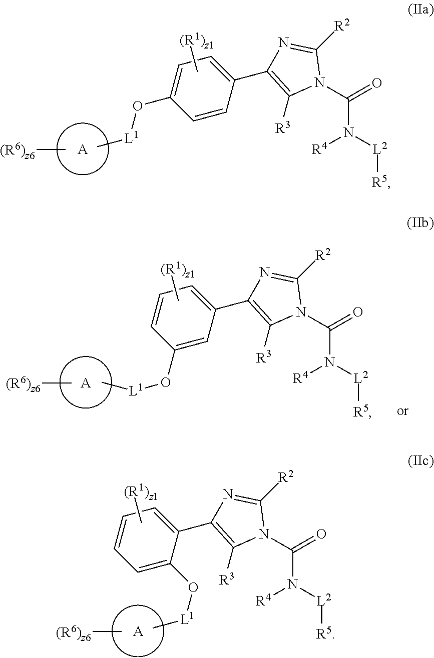

R¹, z1, R², R³, R⁴, R⁵, L¹, and L² are as described herein, including in embodiments.

In embodiments, Ring A is cycloalkyl (e.g., C₃-C₈, C₃-C₆, C₄-C₆, or C₅-C₆), heterocycloalkyl (e.g., 3 to 8 membered, 3 to 6 membered, 4 to 6 membered, 4 to 5 membered, or 5 to 6 membered), aryl (e.g., C₆-C₁₀ or phenyl), or heteroaryl (e.g., 5 to 10 membered, 5 to 9 membered, or 5 to 6 membered).

R⁶ is independently oxo, halogen, —CX⁶₃, —CHX⁶₂, —CH₂X⁶, —OCX⁶₃, —OCH₂X⁶, —OCHX⁶₂, —CN, —SOₙ₆R^{6D}, —S(O)(NH)R^{6D}, —SOᵥ₆NR^{6A}R^{6B}, —ONR^{6A}R^{6B}, —NHC(O)NR^{6C}NR^{6A}R^{6B}, —NHC(O)NR^{6A}R^{6B}, —N(O)ₘ₆, —NR^{6A}R^{6B}, —C(O)R^{6C}, —C(O)OR^{6C}, —C(O)NR^{6A}R^{6B}, —OR^{6D}, —SR^{6D}, —NR^{6A}SO₂R^{6D}, —NR^{6A}C(O)R^{6C}, —NR^{6A}C(O)OR^{6C}, —NR^{6A}OR^{6C}, —SF₅, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); two R⁶ substituents may optionally be joined to form a substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R^{6A}, R^{6B}, R^{6C}, and R^{6D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); R^{6A} and R^{6B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered).

X⁶ is independently —F, —Cl, —Br, or —I.

The symbol n6 is independently an integer from 0 to 4.

The symbols m6 and v6 are independently 1 or 2.

The symbol z6 is an integer from 0 to 11.

In embodiments, the compound has the formula:

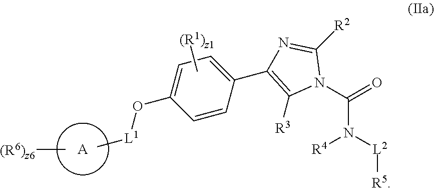

Ring A, R¹, z1, R², R³, R⁴, R⁵, R⁶, z6, L¹, and L² are as described herein, including in embodiments.

In embodiments, the compound has the formula:

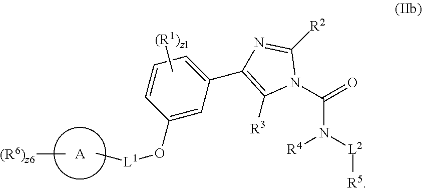

Ring A, R¹, z1, R², R³, R⁴, R⁵, R⁶, z6, L¹, and L² are as described herein, including in embodiments.

In embodiments, the compound has the formula:

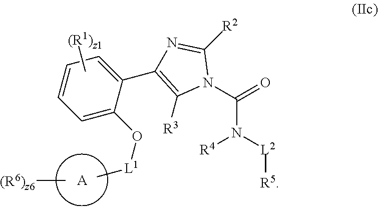

Ring A, R¹, z1, R², R³, R⁴, R⁵, R⁶, z6, L¹, and L² are as described herein, including in embodiments.

In embodiments,

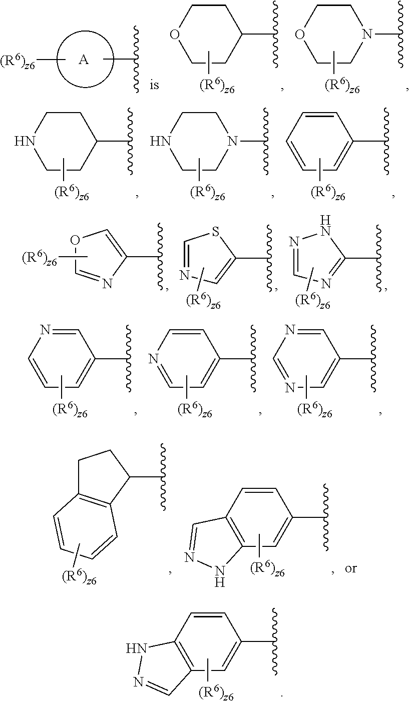

R⁶ z6 are as described herein, including in embodiments.

In embodiments,

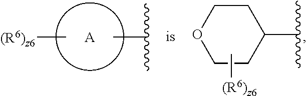

wherein R⁶ and z6 are as described herein, including in embodiments. In embodiments,

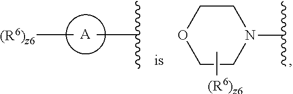

wherein R⁶ and z6 are as described herein, including in embodiments. In embodiments,

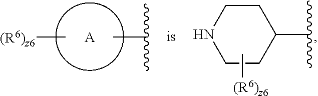

wherein R⁶ and z6 are as described herein, including in embodiments. In embodiments,

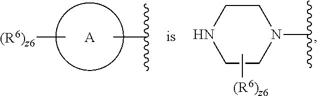

wherein R⁶ and z6 are as described herein, including in embodiments. In embodiments,

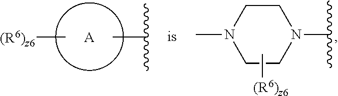

wherein R⁶ and z6 are as described herein, including in embodiments. In embodiments,

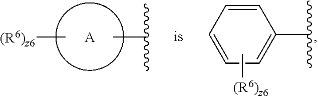

wherein R⁶ and z6 are as described herein, including in embodiments. In embodiments,

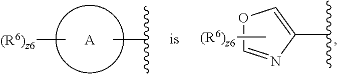

wherein R⁶ and z6 are as described herein, including in embodiments. In embodiments,

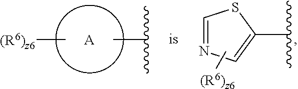

wherein R⁶ and z6 are as described herein, including in embodiments. In embodiments,

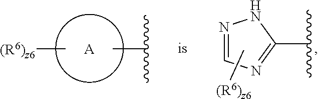

wherein R⁶ and z6 are as described herein, including in embodiments. In embodiments,

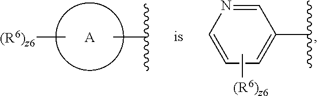

wherein R⁶ and z6 are as described herein, including in embodiments. In embodiments,

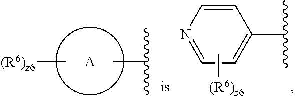

wherein R⁶ and z6 are as described herein, including in embodiments. In embodiments,

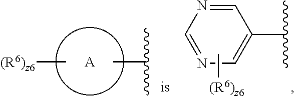

wherein R⁶ and z6 are as described herein, including in embodiments. In embodiments,

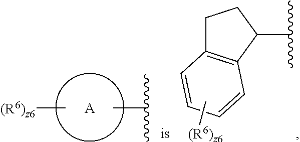

wherein R⁶ and z6 are as described herein, including in embodiments. In embodiments,

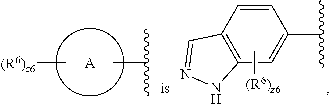

wherein R⁶ and z6 are as described herein, including in embodiments. In embodiments,

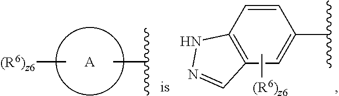

wherein R⁶ and z6 are as described herein, including in embodiments.

In embodiments,

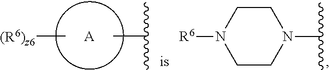

wherein R⁶ is unsubstituted C₁-C₄ alkyl. In embodiments,

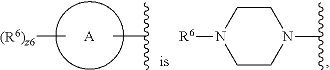

wherein R⁶ is unsubstituted methyl.

In embodiments, a substituted R⁶ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁶ is substituted with a plurality of groups selected from substituent groups, size-limited substituent groups, and lower substituent groups; each substituent group, size-limited substituent group, and/or lower substituent group may optionally be different. In embodiments, when R⁶ is substituted, it is substituted with at least one substituent group. In embodiments, when R⁶ is substituted, it is substituted with at least one size-limited substituent group. In embodiments, when R⁶ is substituted, it is substituted with at least one lower substituent group.

In embodiments, a substituted R^{6A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{6A} is substituted with a plurality of groups selected from substituent groups, size-limited substituent groups, and lower substituent groups; each substituent group, size-limited substituent group, and/or lower substituent group may optionally be different. In embodiments, when R^{6A} is substituted, it is substituted with at least one substituent group. In embodiments, when R^{6A} is substituted, it is substituted with at least one size-limited substituent group. In embodiments, when R^{6A} is substituted, it is substituted with at least one lower substituent group.

In embodiments, a substituted R^{B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{6B} is substituted with a plurality of groups selected from substituent groups, size-limited substituent groups, and lower substituent groups; each substituent group, size-limited substituent group, and/or lower substituent group may optionally be different. In embodiments, when R^{6A} is substituted, it is substituted with at least one substituent group. In embodiments, when R^{6B} is substituted, it is substituted with at least one size-limited substituent group. In embodiments, when R^{6B} is substituted, it is substituted with at least one lower substituent group.

In embodiments, a substituted ring formed when R^{6A} and R^{6B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{6A} and R^{6B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{6A} and R^{6B} substituents bonded to the same nitrogen atom are joined is substituted, it is substituted with at least one substituent group. In embodiments, when the substituted ring formed when R^{6A} and R^{6B} substituents bonded to the same nitrogen atom are joined is substituted, it is substituted with at least one size-limited substituent group. In embodiments, when the substituted ring formed when R^{6A} and R^{6B} substituents bonded to the same nitrogen atom are joined is substituted, it is substituted with at least one lower substituent group.

In embodiments, a substituted R^{6C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{6C} is substituted with a plurality of groups selected from substituent groups, size-limited substituent groups, and lower substituent groups; each substituent group, size-limited substituent group, and/or lower substituent group may optionally be different. In embodiments, when R^{6C} is substituted, it is substituted with at least one substituent group. In embodiments, when R^{6C} is substituted, it is substituted with at least one size-limited substituent group. In embodiments, when R^{6C} is substituted, it is substituted with at least one lower substituent group.

In embodiments, a substituted R^{6D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{6D} is substituted with a plurality of groups selected from substituent groups, size-limited substituent groups, and lower substituent groups; each substituent group, size-limited substituent group, and/or lower substituent group may optionally be different. In embodiments, when R^{D} is substituted, it is substituted with at least one substituent group. In embodiments, when R^{6D} is substituted, it is substituted with at least one size-limited substituent group. In embodiments, when R^{6D} is substituted, it is substituted with at least one lower substituent group.

In embodiments, R⁶ is independently halogen, —CN, —SOₙ₆R^{6D}, —S(O)(NH)R^{6D}, —SOᵥ₆NR^{6A}R^{6B}, —NR^{6A}R^{6B}, —C(O)NR^{6A}R^{6B}, —OR^{6D}, —SR^{6D}, —NR^{6A}SO₂R^{6D}, —NR^{6A}C(O)R^{6C}, substituted or unsubstituted C₁-C₆ alkyl, substituted or unsubstituted 2 to 6 membered heteroalkyl, or substituted or unsubstituted 3 to 6 membered heterocycloalkyl. In embodiments, R⁶ is independently halogen, —CF₃, —CN, —SOₙ₆R^{6D}, —S(O)(NH)R^{6D}, —SOᵥ₆NR^{6A}R^{6B}, —NR^{6A}R^{6B}, —C(O)NR^{6A}R^{6B}, —OR^{6D}, —SR^{6D}, —NR^{6A}SO₂R^{6D}, —NR^{6A}C(O)R^{6C}, substituted or unsubstituted C₁-C₆ alkyl, substituted or unsubstituted 2 to 6 membered heteroalkyl, or substituted or unsubstituted 3 to 6 membered heterocycloalkyl. In embodiments, R⁶ is independently halogen. In embodiments, R⁶ is independently —F. In embodiments, R⁶ is independently —Cl. In embodiments, R⁶ is independently —Br. In embodiments, R⁶ is independently —I. In embodiments, R⁶ is independently —CF₃. In embodiments, R⁶ is independently —CN. In embodiments, R⁶ is independently —SOₙ₆R^{6D}. In embodiments, R⁶ is independently —S(O)(NH)R^{6D}. In embodiments, R⁶ is independently —SOᵥ₆NR^{6A}R^{6B}. In embodiments, R⁶ is independently —NR^{6A}R^{6B}. In embodiments, R⁶ is independently —C(O)NR^{6A}R^{6B}. In embodiments, R⁶ is independently —OR^{6D}. In embodiments, R⁶ is independently —SR^{6D}. In embodiments, R⁶ is independently —NR^{6A}SO₂R^{6D}. In embodiments, R⁶ is independently —NR^{6A}C(O)R^{6C}. In embodiments, R⁶ is independently substituted or unsubstituted C₁-C₆ alkyl. In embodiments, R⁶ is independently substituted or unsubstituted 2 to 6 membered heteroalkyl. In embodiments, R⁶ is independently substituted or unsubstituted 3 to 6 membered heterocycloalkyl.

In embodiments, R^{6A} and R^{6B} are independently hydrogen, substituted or unsubstituted C₁-C₄ alkyl, substituted or unsubstituted 2 to 6 membered heteroalkyl, or substituted or unsubstituted 3 to 6 membered heterocycloalkyl.

In embodiments, R^{6A} is independently hydrogen, substituted or unsubstituted C₁-C₄ alkyl, substituted or unsubstituted 2 to 6 membered heteroalkyl, or substituted or unsubstituted 3 to 6 membered heterocycloalkyl. In embodiments, R^{6A} is independently hydrogen. In embodiments, R^{6A} is independently substituted or unsubstituted C₁-C₄ alkyl. In embodiments, R^{6A} is independently unsubstituted methyl. In embodiments, R^{6A} is independently unsubstituted ethyl. In embodiments, R^{6A} is independently unsubstituted propyl. In embodiments, R^{6A} is independently unsubstituted n-propyl. In embodiments, R^{6A} is independently unsubstituted isopropyl. In embodiments, R^{6A} is independently unsubstituted butyl. In embodiments, R^{6A} is independently unsubstituted n-butyl. In embodiments, R^{6A} is independently unsubstituted isobutyl. In embodiments, R^{6A} is independently unsubstituted tert-butyl. In embodiments, R^{6A} is independently oxo-substituted C₂-C₄ alkyl. In embodiments, R^{6A} is independently oxo-substituted ethyl. In embodiments, R^{6A} is independently oxo-substituted propyl. In embodiments, R^{6A} is independently oxo-substituted n-propyl. In embodiments, R^{6A} is independently oxo-substituted butyl. In embodiments, R^{6A} is independently oxo-substituted n-butyl. In embodiments, R^{6A} is independently substituted or unsubstituted 2 to 6 membered heteroalkyl.

In embodiments, R^{6A} is independently

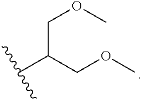 [SMILES: *C(COC)COC]

In embodiments, R^{6A} is independently

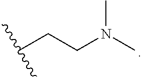 [SMILES: *CCN(C)C]

In embodiments, R^{6A} is independently substituted or unsubstituted 3 to 6 membered heterocycloalkyl. In embodiments, R^{6A} is independently substituted azetidinyl. In embodiments, R^{6A} is independently

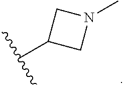 [SMILES: *C1CN(C)C1]

In embodiments, R^{6B} is independently hydrogen, substituted or unsubstituted C₁-C₄ alkyl, substituted or unsubstituted 2 to 6 membered heteroalkyl, or substituted or unsubstituted 3 to 6 membered heterocycloalkyl. In embodiments, R^{6B} is independently hydrogen. In embodiments, R* is independently substituted or unsubstituted C₁-C₄ alkyl. In embodiments, R* is independently unsubstituted methyl. In embodiments, R^{6B} is independently unsubstituted ethyl. In embodiments, R^{6B} is independently unsubstituted propyl. In embodiments, R^{6B} is independently unsubstituted n-propyl. In embodiments, R^{6B} is independently unsubstituted isopropyl. In embodiments, R* is independently unsubstituted butyl. In embodiments, R^{6B} is independently unsubstituted n-butyl. In embodiments, R^{6B} is independently unsubstituted isobutyl. In embodiments, R^{6B} is independently unsubstituted tert-butyl. In embodiments, R^{6B} is independently oxo-substituted C₂-C₄ alkyl. In embodiments, R^{6B} is independently oxo-substituted ethyl. In embodiments, R^{6B} is independently oxo-substituted propyl. In embodiments, R^{6B} is independently oxo-substituted n-propyl. In embodiments, R^{6B} is independently oxo-substituted butyl. In embodiments, R^{6B} is independently oxo-substituted n-butyl. In embodiments, R^{6B} is independently substituted or unsubstituted 2 to 6 membered heteroalkyl. In embodiments, R^{6B} is independently

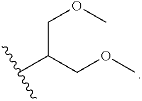 [SMILES: *C(COC)COC]

In embodiments, R^{6B} is independently

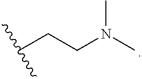 [SMILES: *CCN(C)C]

In embodiments, R^{6B} is independently substituted or unsubstituted 3 to 6 membered heterocycloalkyl. In embodiments, R^{6B} is independently substituted azetidinyl. In embodiments, R^{6B} is independently

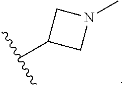 [SMILES: *C1CN(C)C1]

In embodiments, R^{6A} and R^{6B} substituents bonded to the same nitrogen atom are joined to form a substituted or unsubstituted 3 to 6 membered heterocycloalkyl. In embodiments, R^{6A} and R^{6B} substituents bonded to the same nitrogen atom are joined to form a substituted or unsubstituted azetidinyl. In embodiments, R^{6A} and R^{6B} substituents bonded to the same nitrogen atom are joined to form a substituted or unsubstituted morpholinyl. In embodiments, R^{6A} and R^{6B} substituents bonded to the same nitrogen atom are joined to form a substituted or unsubstituted piperazinyl.

In embodiments, R^{6C} is independently unsubstituted C₃-C₆ cycloalkyl or substituted or unsubstituted 3 to 6 membered heterocycloalkyl. In embodiments, R^{6C} is independently unsubstituted cyclopropyl. In embodiments, R^{6C} is independently substituted or unsubstituted oxetanyl. In embodiments, R^{6C} is independently substituted or unsubstituted azetidinyl.

In embodiments, R^{6D} is independently hydrogen, —CHF₂, substituted or unsubstituted C₁-C₄ alkyl, unsubstituted 2 to 6 membered heteroalkyl, or substituted or unsubstituted 3 to 6 membered heterocycloalkyl. In embodiments, R^{6D} is independently hydrogen. In embodiments, R^{6D} is independently —CHF₂. In embodiments, R^{6D} is independently substituted or unsubstituted C₁-C₄ alkyl. In embodiments, R^{6D} is independently unsubstituted methyl. In embodiments, R^{6D} is independently unsubstituted ethyl. In embodiments, R^{6D} is independently unsubstituted propyl. In embodiments, R^{6D} is independently unsubstituted n-propyl. In embodiments, R^{6D} is independently unsubstituted isopropyl. In embodiments, R^{6D} is independently unsubstituted butyl. In embodiments, R^{6D} is independently unsubstituted n-butyl. In embodiments, R^{6D} is independently unsubstituted isobutyl. In embodiments, R^{D} is independently unsubstituted tert-butyl. In embodiments, R^{6D} is independently unsubstituted 2 to 6 membered heteroalkyl. In embodiments, R^{6D} is independently substituted or unsubstituted 3 to 6 membered heterocycloalkyl.

In embodiments, R⁶ is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R⁶ is independently hydrogen or unsubstituted C₁-C₆ alkyl. In embodiments, R⁶ is independently hydrogen. In embodiments, R⁶ is independently unsubstituted methyl. In embodiments, R⁶ is independently unsubstituted ethyl. In embodiments, R⁶ is independently unsubstituted propyl. In embodiments, R⁶ is independently unsubstituted n-propyl. In embodiments, R⁶ is independently unsubstituted isopropyl. In embodiments, R⁶ is independently unsubstituted butyl. In embodiments, R⁶ is independently unsubstituted n-butyl. In embodiments, R⁶ is independently unsubstituted isobutyl. In embodiments, R⁶ is independently unsubstituted tert-butyl. In embodiments, R⁶ is independently unsubstituted pentyl. In embodiments, R⁶ is independently unsubstituted hexyl.

In embodiments, R⁶ is independently —F, —CN, —SO₂CH₃, —SO₂NH₂, —SO₂NHCH₃, —S(O)CH₃, —S(O)(NH)CH₃, —NH₂, —C(O)NH₂, —SCH₃, —OH, —NHSO₂CH₃, —NHSO₂CHF₂, —CH₃,

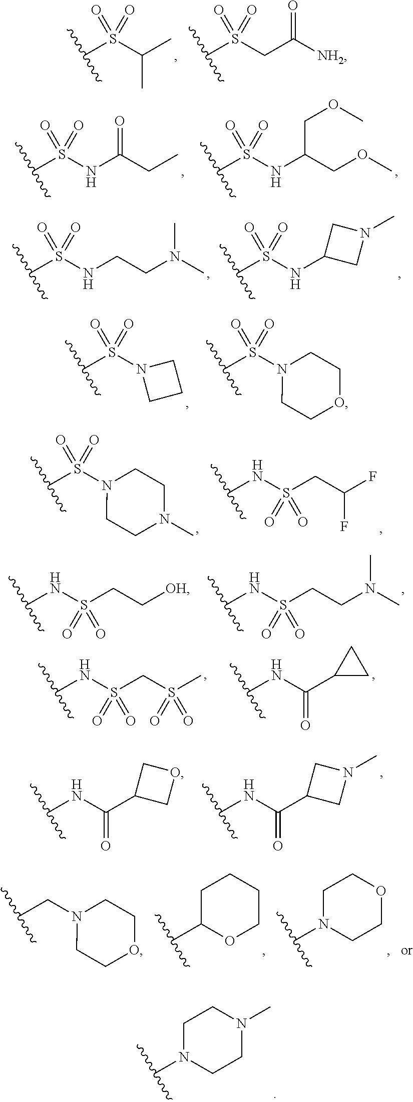

In embodiments, R⁶ is independently —F, —CF₃, —CN, —SO₂CH₃, —SO₂NH₂, —SO₂NHCH₃, —S(O)CH₃, —S(O)(NH)CH₃, —NH₂, —C(O)NH₂, —SCH₃, —OH, —NHSO₂CH₃, —NHSO₂CHF₂, —CH₃,

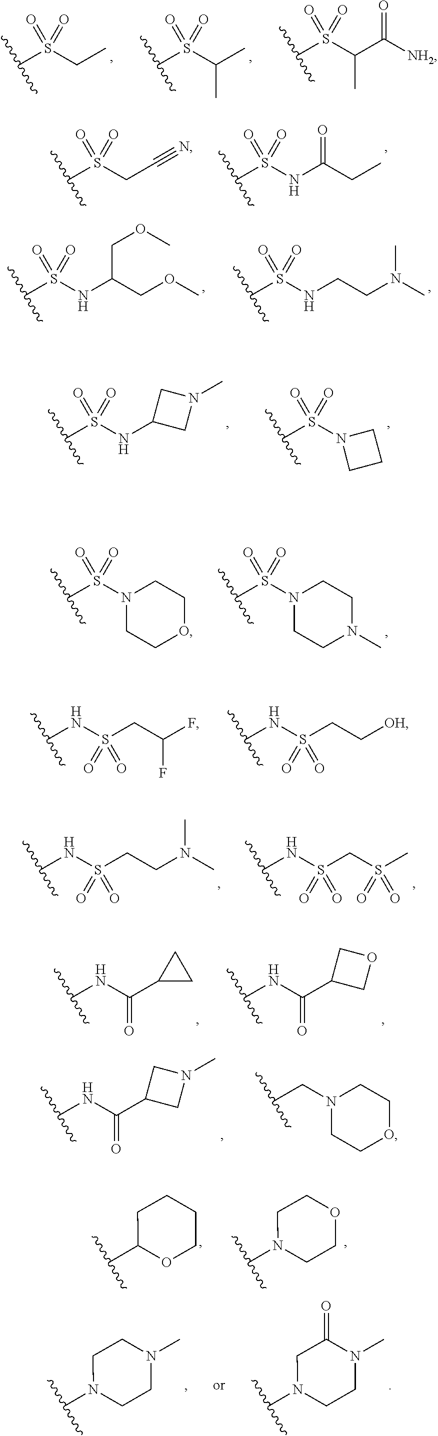

In embodiments, R⁶ is independently —F. In embodiments, R⁶ is independently —CF₃. In embodiments, R⁶ is independently —CN. In embodiments, R⁶ is independently —SO₂CH₃. In embodiments, R⁶ is independently —SO₂NH₂. In embodiments, R⁶ is independently —SO₂NHCH₃. In embodiments, R⁶ is independently —S(O)CH₃. In embodiments, R⁶ is independently —S(O)(NH)CH₃. In embodiments, R⁶ is independently —NH₂. In embodiments, R⁶ is independently —C(O)NH₂. In embodiments, R⁶ is independently —SCH₃. In embodiments, R⁶ is independently —OH. In embodiments, R⁶ is independently —NHSO₂CH₃. In embodiments, R⁶ is independently —NHSO₂CHF₂. In embodiments, R⁶ is independently —CH₃. In embodiments, R⁶ is independently

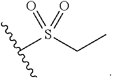 [SMILES: *S(=O)(=O)CC]

In embodiments, R⁶ is independently

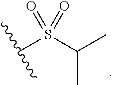 [SMILES: *S(=O)(=O)C(C)C]

In embodiments, R⁶ is independently

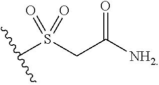 [SMILES: *S(=O)(=O)CC(N)=O]

In embodiments, R⁶ is independently

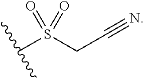 [SMILES: *S(=O)(=O)CC#N]

In embodiments, R⁶ is independently

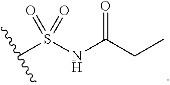 [SMILES: *S(=O)(=O)NC(=O)CC]

In embodiments, R⁶ is independently

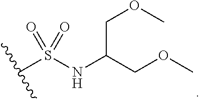 [SMILES: *S(=O)(=O)NC(COC)COC]

In embodiments, R⁶ is independently

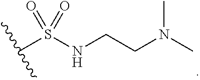 [SMILES: *S(=O)(=O)NCCN(C)C]

In embodiments, R⁶ is independently

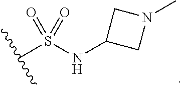 [SMILES: *S(=O)(=O)NC1CN(C)C1]

In embodiments, R⁶ is independently

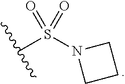 [SMILES: *S(=O)(=O)N1CCC1]

In embodiments, R⁶ is independently

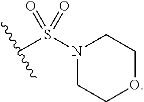 [SMILES: *S(=O)(=O)N1CCOCC1]

In embodiments, R⁶ is independently

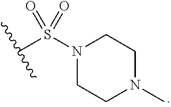 [SMILES: *S(=O)(=O)N1CCN(C)CC1]

In embodiments, R⁶ is independently

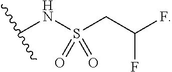 [SMILES: *NS(=O)(=O)CC(F)F]

In embodiments, R⁶ is independently

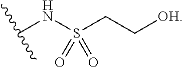 [SMILES: *NS(=O)(=O)CCO]

In embodiments, R⁶ is independently

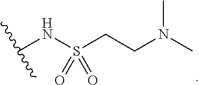 [SMILES: *NS(=O)(=O)CCN(C)C]

In embodiments, R⁶ is independently

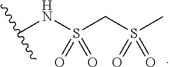 [SMILES: *NS(=O)(=O)CS(C)(=O)=O]

In embodiments, R⁶ is independently

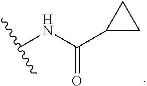 [SMILES: *NC(=O)C1CC1]

In embodiments, R⁶ is independently

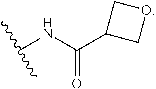 [SMILES: *NC(=O)C1COC1]

In embodiments, R⁶ is independently

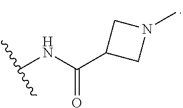 [SMILES: *NC(=O)C1CN(C)C1]

In embodiments, R⁶ is independently

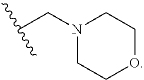 [SMILES: *CN1CCOCC1]

In embodiments, R⁶ is independently

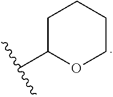 [SMILES: *C1CCCCO1]

In embodiments, R⁶ is independently

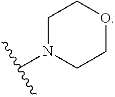 [SMILES: *N1CCOCC1]

In embodiments, R⁶ is independently

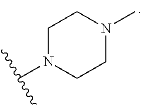 [SMILES: *N1CCN(C)CC1]

In embodiments, R⁶ is independently

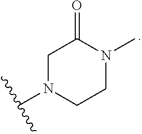 [SMILES: *N1CCN(C)C(=O)C1]

In embodiments, z6 is 0. In embodiments, z6 is 1. In embodiments, z6 is 2. In embodiments, z6 is 3. In embodiments, z6 is 4. In embodiments, z6 is 5. In embodiments, z6 is 6. In embodiments, z6 is 7. In embodiments, z6 is 8. In embodiments, z6 is 9. In embodiments, z6 is 10. In embodiments, z6 is 11.

In embodiments, is,

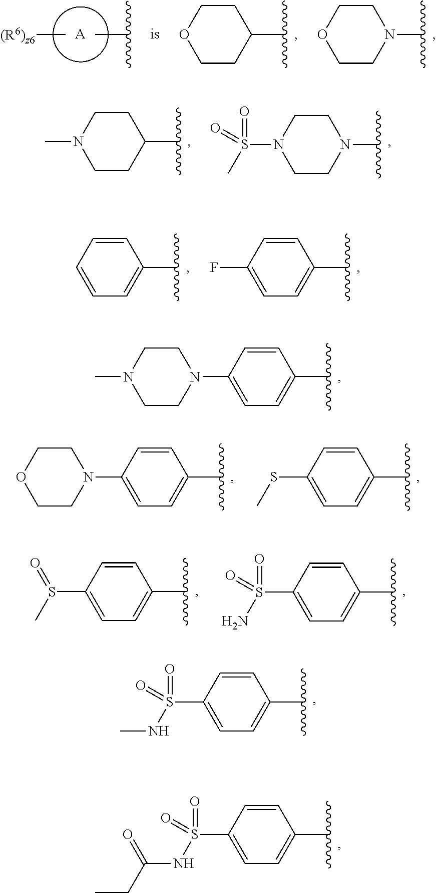

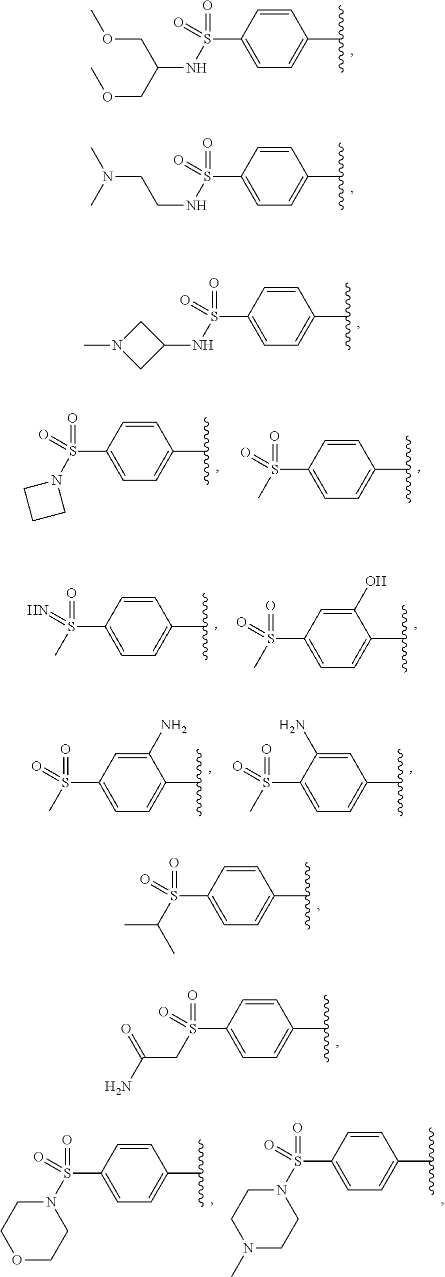

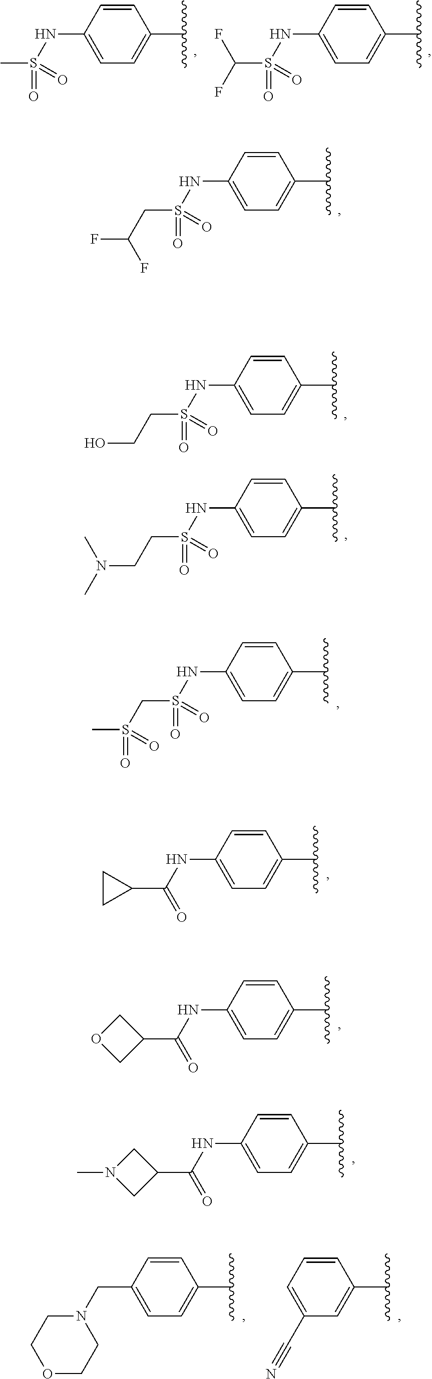

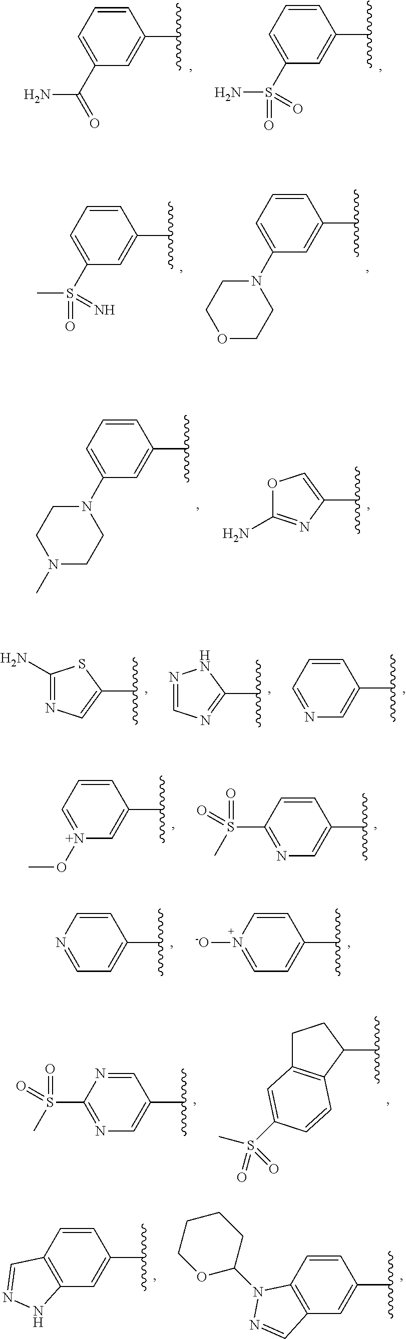

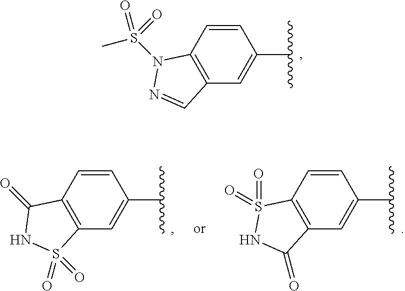 [SMILES: *c1ccc2c(cnn2S(C)(=O)=O)c1 ; *c1ccc2c(c1)S(=O)(=O)NC2=O ; *c1ccc2c(c1)C(=O)NS2(=O)=O]

In embodiments,

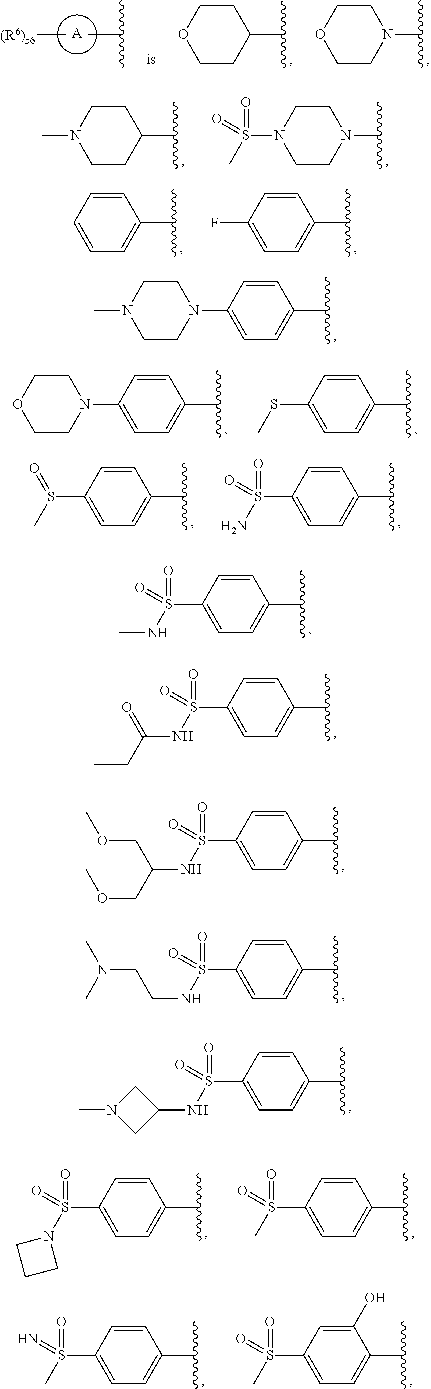

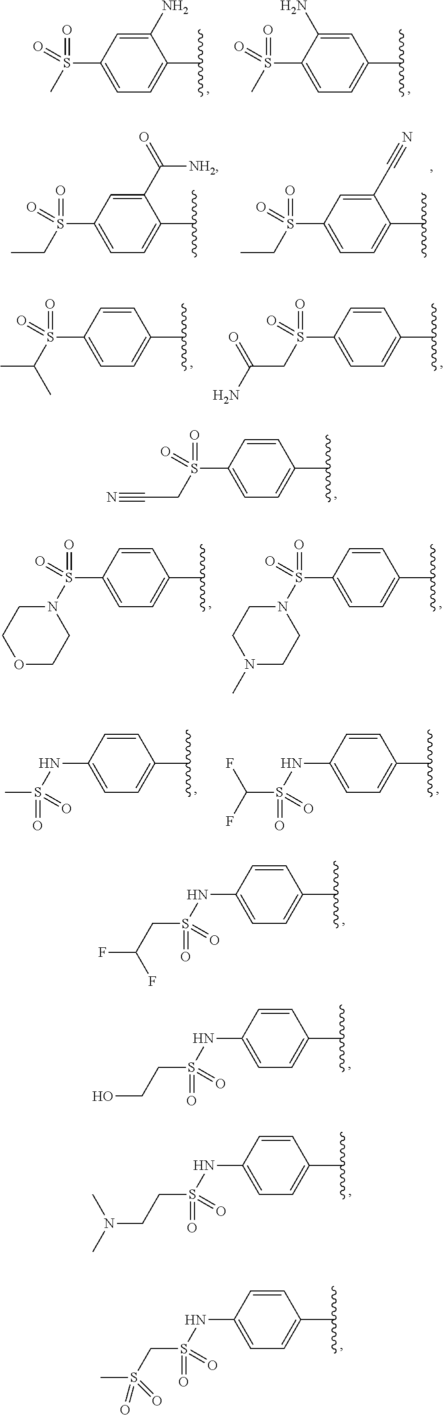

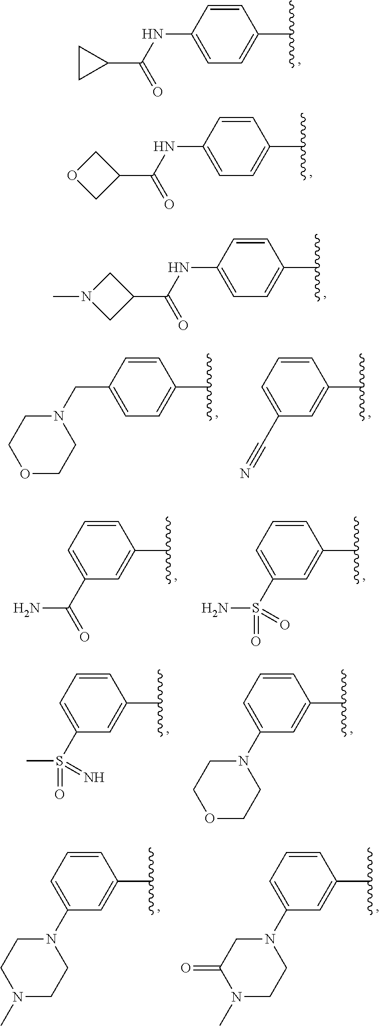

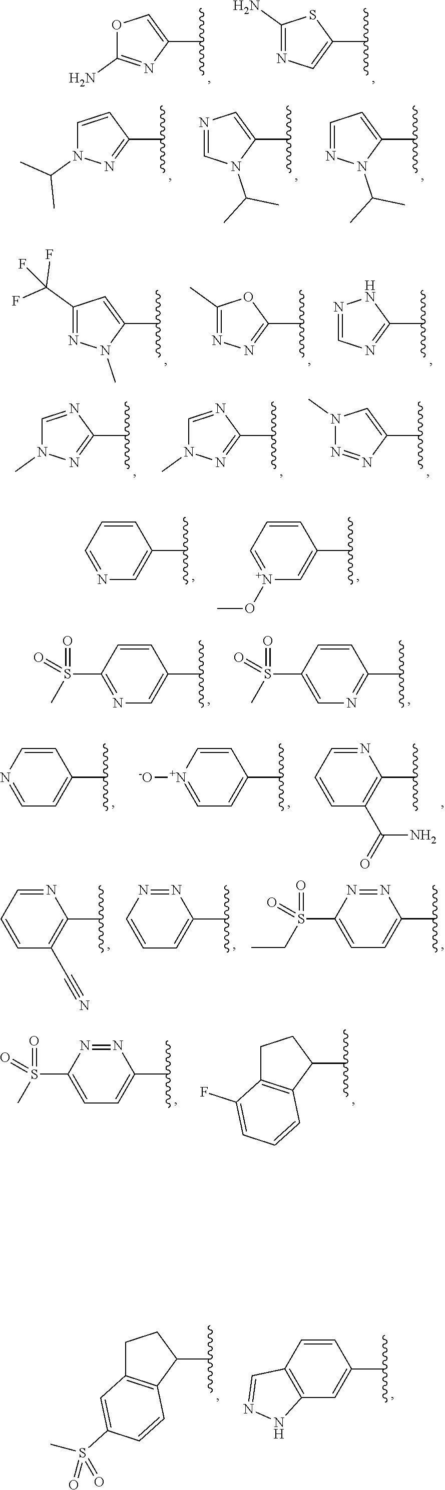

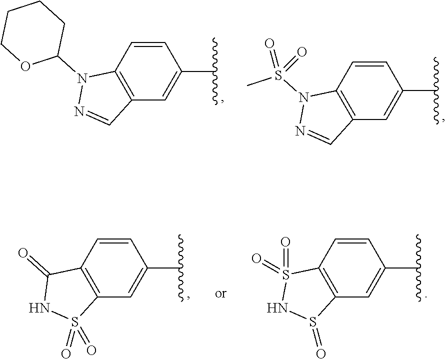

In embodiments,

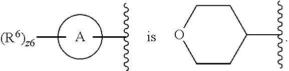

In embodiments

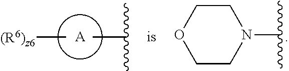

In embodiments,

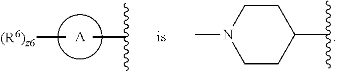

In embodiments,

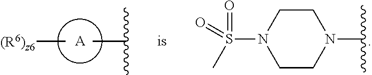

In embodiments,

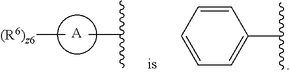

In embodiments,

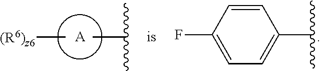

In embodiments,

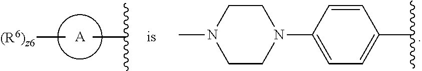

In embodiments,

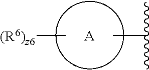

is

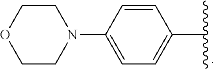 [SMILES: *c1ccc(N2CCOCC2)cc1]

In embodiments,

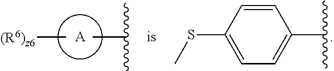

In embodiments,

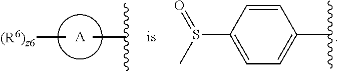

In embodiments,

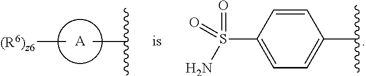

In embodiments,

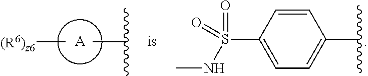

In embodiments,

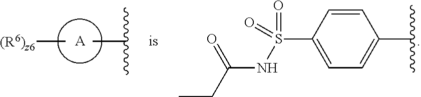

In embodiments,

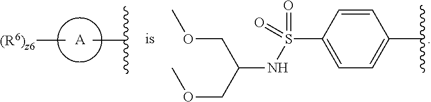

In embodiments,

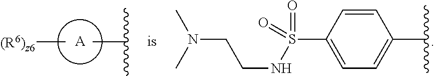

In embodiments,

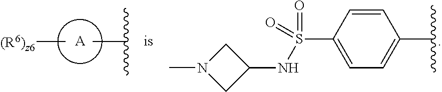

In embodiments,

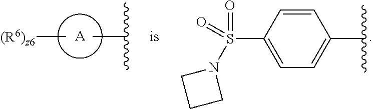

In embodiments,

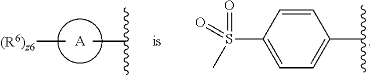

In embodiments,

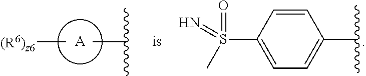

In embodiments,

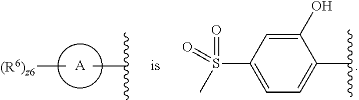

In embodiments,

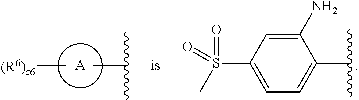

In embodiments,

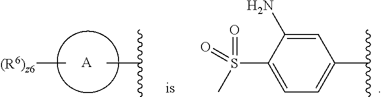

In embodiments,

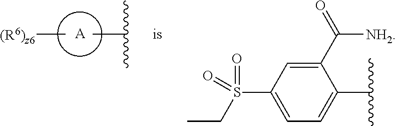

In embodiments,

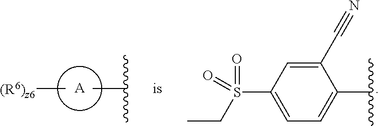

In embodiments,

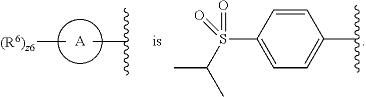

In embodiments,

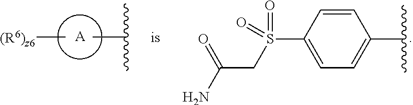

In embodiments,

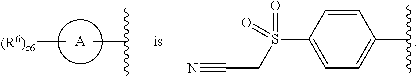

In embodiments,

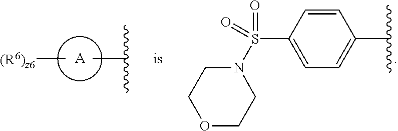

In embodiments,

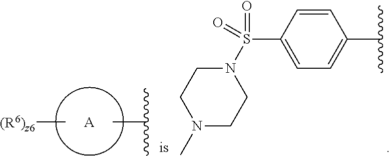

In embodiments,

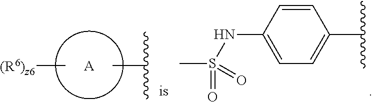

In embodiments,

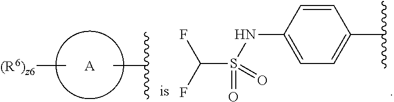

In embodiments,

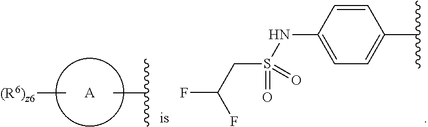

In embodiments,

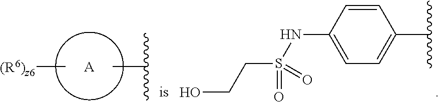

In embodiments,

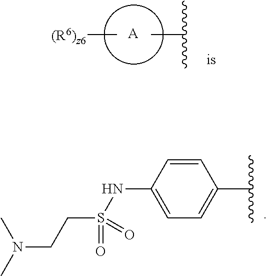

In embodiments,

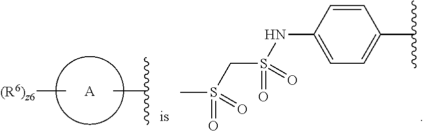

In embodiments,

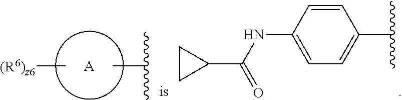

In embodiments,

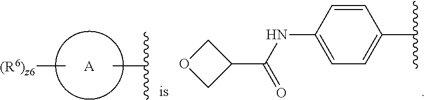

In embodiments,

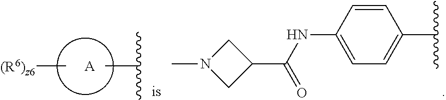

In embodiments,

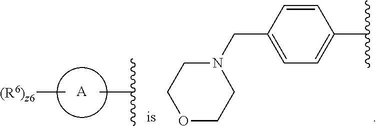

In embodiments,

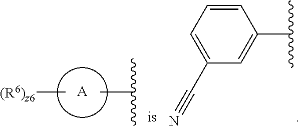

In embodiments,

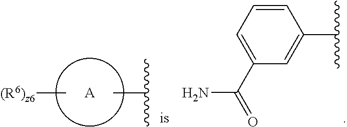

In embodiments,

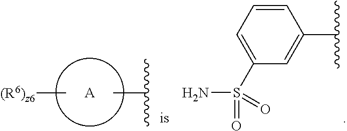

In embodiments,

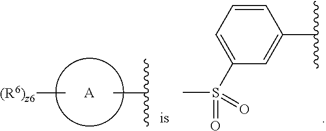

In embodiments,

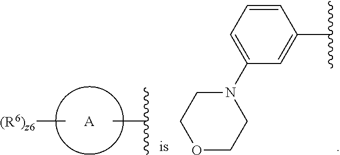

In embodiments,

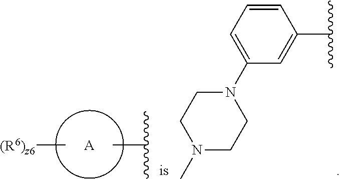

In embodiments,

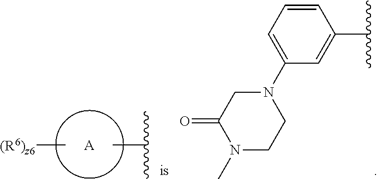

In embodiments,

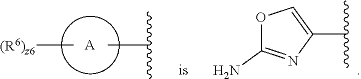

In embodiments,

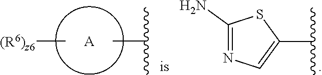

In embodiments,

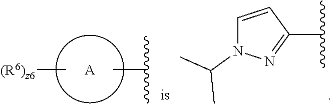

In embodiments,

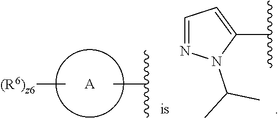

In embodiments,

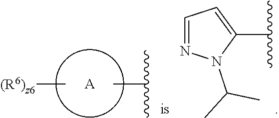

In embodiments,

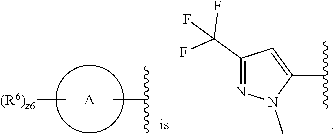

In embodiments,

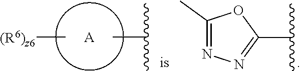

In embodiments,

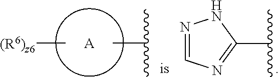

In embodiments,

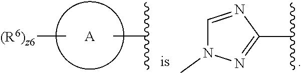

In embodiments,

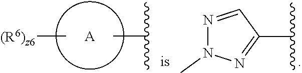

In embodiments,

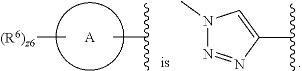

In embodiments,

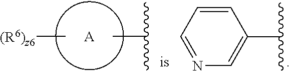

In embodiments,

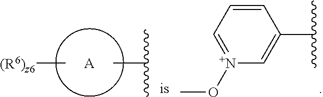

In embodiments,

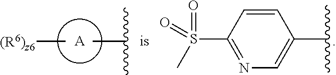

In embodiments,

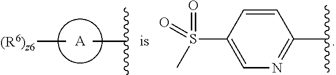

In embodiments,

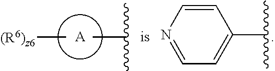

In embodiments,

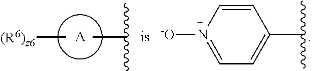

In embodiments,

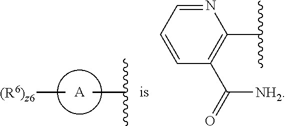

In embodiments,

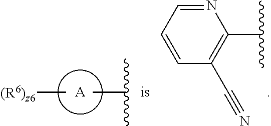

In embodiments,

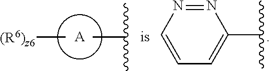

In embodiments,

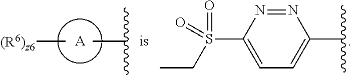

In embodiments,

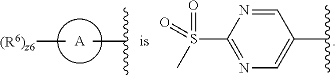

In embodiments,

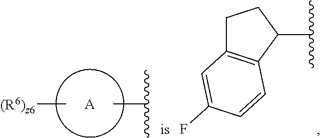

In embodiments,

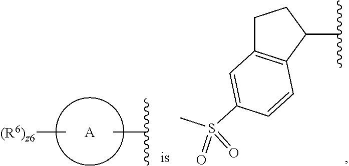

In embodiments,

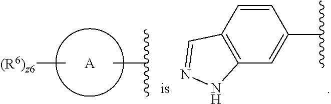

In embodiments,

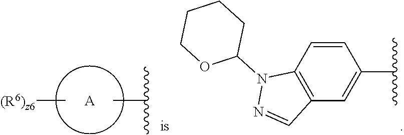

In embodiments,

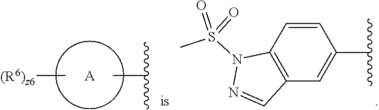

In embodiments,

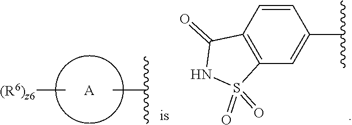

In embodiments,

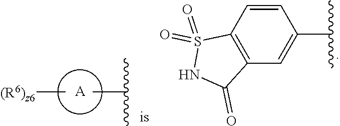

In embodiments, a substituted L¹ (e.g., substituted alkylene and/or substituted heteroalkylene) is substituted with at least one substituent group, size-limited substituent group, or lower substituent group; wherein if the substituted L¹ is substituted with a plurality of groups selected from substituent groups, size-limited substituent groups, and lower substituent groups; each substituent group, size-limited substituent group, and/or lower substituent group may optionally be different. In embodiments, when L¹ is substituted, it is substituted with at least one substituent group. In embodiments, when L¹ is substituted, it is substituted with at least one size-limited substituent group. In embodiments, when L¹ is substituted, it is substituted with at least one lower substituent group.

In embodiments, L¹ is a bond, substituted or unsubstituted alkylene (e.g., C₁-C₈, C₁-C₆, C₁-C₄, or C₁-C₂), or substituted or unsubstituted heteroalkylene (e.g., 2 to 8 membered, 2 to 6 membered, 4 to 6 membered, 2 to 3 membered, or 4 to 5 membered).

In embodiments, L¹ is a bond, substituted or unsubstituted C₁-C₄ alkylene, or substituted or unsubstituted 2 to 4 membered heteroalkylene. In embodiments, L¹ is a bond, —C(O)—, substituted or unsubstituted C₁-C₄ alkylene, or substituted or unsubstituted 2 to 4 membered heteroalkylene. In embodiments, L¹ is a bond. In embodiments, L¹ is —C(O)—. In embodiments, L¹ is substituted or unsubstituted C₁-C₄ alkylene. In embodiments, L¹ is unsubstituted C₁-C₄ alkylene. In embodiments, L¹ is unsubstituted methylene. In embodiments, L¹ is unsubstituted ethylene. In embodiments, L¹ is unsubstituted propylene. In embodiments, L¹ is unsubstituted n-propylene. In embodiments, L¹ is unsubstituted isopropylene. In embodiments, L¹ is unsubstituted butylene. In embodiments, L¹ is unsubstituted n-butylene. In embodiments, L¹ is unsubstituted isobutylene. In embodiments, L¹ is unsubstituted tert-butylene. In embodiments, L¹ is substituted C₁-C₄ alkylene. In embodiments, L¹ is substituted methylene. In embodiments, L¹ is substituted ethylene. In embodiments, L¹ is substituted propylene. In embodiments, L¹ is substituted n-propylene. In embodiments, L¹ is substituted isopropylene. In embodiments, L¹ is substituted butylene. In embodiments, L¹ is substituted n-butylene. In embodiments, L¹ is substituted isobutylene. In embodiments, L¹ is substituted tert-butylene. In embodiments, L¹ is substituted or unsubstituted 2 to 4 membered heteroalkylene.

In embodiments, L¹ is a bond,

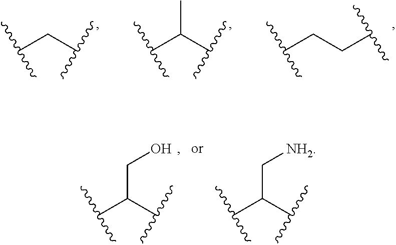

In embodiments, L¹ is a bond. In embodiments, L¹ is

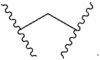 [SMILES: *C*]

In embodiments, L¹ is

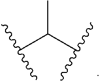 [SMILES: *C(*)C]

In embodiments, L¹ is

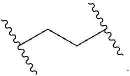 [SMILES: *CC*]

In embodiments, L¹ is

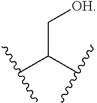 [SMILES: *C(*)CO]

In embodiments, L¹ is

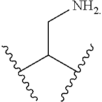 [SMILES: *C(*)CN]

In embodiments, L¹ is a bond, —C(O)—,

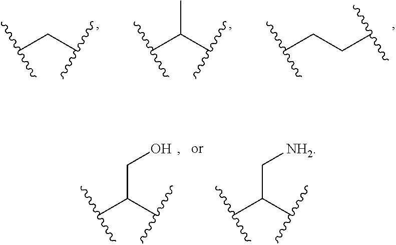

In embodiments, a substituted R¹ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹ is substituted with a plurality of groups selected from substituent groups, size-limited substituent groups, and lower substituent groups; each substituent group, size-limited substituent group, and/or lower substituent group may optionally be different. In embodiments, when R¹ is substituted, it is substituted with at least one substituent group. In embodiments, when R¹ is substituted, it is substituted with at least one size-limited substituent group. In embodiments, when R¹ is substituted, it is substituted with at least one lower substituent group.

In embodiments, a substituted R^{1A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1A} is substituted with a plurality of groups selected from substituent groups, size-limited substituent groups, and lower substituent groups; each substituent group, size-limited substituent group, and/or lower substituent group may optionally be different. In embodiments, when R^{1A} is substituted, it is substituted with at least one substituent group. In embodiments, when R^{1A} is substituted, it is substituted with at least one size-limited substituent group. In embodiments, when R^{1A} is substituted, it is substituted with at least one lower substituent group.

In embodiments, a substituted R^{1B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1B} is substituted with a plurality of groups selected from substituent groups, size-limited substituent groups, and lower substituent groups; each substituent group, size-limited substituent group, and/or lower substituent group may optionally be different. In embodiments, when R^{1B} is substituted, it is substituted with at least one substituent group. In embodiments, when R^{1B} is substituted, it is substituted with at least one size-limited substituent group. In embodiments, when R^{1B} is substituted, it is substituted with at least one lower substituent group.

In embodiments, a substituted ring formed when R^{1A} and R^{1B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{1A} and R^{1B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{1A} and R^{1B} substituents bonded to the same nitrogen atom are joined is substituted, it is substituted with at least one substituent group. In embodiments, when the substituted ring formed when R^{1A} and R^{1B} substituents bonded to the same nitrogen atom are joined is substituted, it is substituted with at least one size-limited substituent group. In embodiments, when the substituted ring formed when R^{1A} and R^{1B} substituents bonded to the same nitrogen atom are joined is substituted, it is substituted with at least one lower substituent group.

In embodiments, a substituted R^{1C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1C} is substituted with a plurality of groups selected from substituent groups, size-limited substituent groups, and lower substituent groups; each substituent group, size-limited substituent group, and/or lower substituent group may optionally be different. In embodiments, when R^{1C} is substituted, it is substituted with at least one substituent group. In embodiments, when R^{1C} is substituted, it is substituted with at least one size-limited substituent group. In embodiments, when R^{1C} is substituted, it is substituted with at least one lower substituent group.

In embodiments, a substituted R^{1D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1D} is substituted with a plurality of groups selected from substituent groups, size-limited substituent groups, and lower substituent groups; each substituent group, size-limited substituent group, and/or lower substituent group may optionally be different. In embodiments, when R^{1D} is substituted, it is substituted with at least one substituent group. In embodiments, when R^{1D} is substituted, it is substituted with at least one size-limited substituent group. In embodiments, when R^{1D} is substituted, it is substituted with at least one lower substituent group.

In embodiments, R¹ is independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, z1 is 0. In embodiments, z1 is 1. In embodiments, z1 is 2. In embodiments, z1 is 3. In embodiments, z1 is 4.

In embodiments, a substituted R² (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R² is substituted with a plurality of groups selected from substituent groups, size-limited substituent groups, and lower substituent groups; each substituent group, size-limited substituent group, and/or lower substituent group may optionally be different. In embodiments, when R² is substituted, it is substituted with at least one substituent group. In embodiments, when R² is substituted, it is substituted with at least one size-limited substituent group. In embodiments, when R² is substituted, it is substituted with at least one lower substituent group.

In embodiments, a substituted R^{2A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2A} is substituted with a plurality of groups selected from substituent groups, size-limited substituent groups, and lower substituent groups; each substituent group, size-limited substituent group, and/or lower substituent group may optionally be different. In embodiments, when R^{2A} is substituted, it is substituted with at least one substituent group. In embodiments, when R^{2A} is substituted, it is substituted with at least one size-limited substituent group. In embodiments, when R^{2A} is substituted, it is substituted with at least one lower substituent group.

In embodiments, a substituted R^{2B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2B} is substituted with a plurality of groups selected from substituent groups, size-limited substituent groups, and lower substituent groups; each substituent group, size-limited substituent group, and/or lower substituent group may optionally be different. In embodiments, when R^{2B} is substituted, it is substituted with at least one substituent group. In embodiments, when R^{2B} is substituted, it is substituted with at least one size-limited substituent group. In embodiments, when R^{2B} is substituted, it is substituted with at least one lower substituent group.

In embodiments, a substituted ring formed when R^{2A} and R^{2B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{2A} and R^{2B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{2A} and R^{2B} substituents bonded to the same nitrogen atom are joined is substituted, it is substituted with at least one substituent group. In embodiments, when the substituted ring formed when R^{2A} and R^{2B} substituents bonded to the same nitrogen atom are joined is substituted, it is substituted with at least one size-limited substituent group. In embodiments, when the substituted ring formed when R^{2A} and R^{2B} substituents bonded to the same nitrogen atom are joined is substituted, it is substituted with at least one lower substituent group.

In embodiments, a substituted R^{2C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2C} is substituted with a plurality of groups selected from substituent groups, size-limited substituent groups, and lower substituent groups; each substituent group, size-limited substituent group, and/or lower substituent group may optionally be different. In embodiments, when R^{2C} is substituted, it is substituted with at least one substituent group. In embodiments, when R^{2C} is substituted, it is substituted with at least one size-limited substituent group. In embodiments, when R^{2C} is substituted, it is substituted with at least one lower substituent group.

In embodiments, a substituted R^{2D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2D} is substituted with a plurality of groups selected from substituent groups, size-limited substituent groups, and lower substituent groups; each substituent group, size-limited substituent group, and/or lower substituent group may optionally be different. In embodiments, when R^{2D} is substituted, it is substituted with at least one substituent group. In embodiments, when R^{2D} is substituted, it is substituted with at least one size-limited substituent group. In embodiments, when R^{2D} is substituted, it is substituted with at least one lower substituent group.

In embodiments, R² is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R² is hydrogen or halogen. In embodiments, R² is hydrogen. In embodiments, R² is halogen.

In embodiments, a substituted R³ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R³ is substituted with a plurality of groups selected from substituent groups, size-limited substituent groups, and lower substituent groups; each substituent group, size-limited substituent group, and/or lower substituent group may optionally be different. In embodiments, when R³ is substituted, it is substituted with at least one substituent group. In embodiments, when R³ is substituted, it is substituted with at least one size-limited substituent group. In embodiments, when R³ is substituted, it is substituted with at least one lower substituent group.

In embodiments, a substituted R^{3A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3A} is substituted with a plurality of groups selected from substituent groups, size-limited substituent groups, and lower substituent groups; each substituent group, size-limited substituent group, and/or lower substituent group may optionally be different. In embodiments, when R^{3A} is substituted, it is substituted with at least one substituent group. In embodiments, when R^{3A} is substituted, it is substituted with at least one size-limited substituent group. In embodiments, when R^{3A} is substituted, it is substituted with at least one lower substituent group.

In embodiments, a substituted R^{3B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3B} is substituted with a plurality of groups selected from substituent groups, size-limited substituent groups, and lower substituent groups; each substituent group, size-limited substituent group, and/or lower substituent group may optionally be different. In embodiments, when R^{3B} is substituted, it is substituted with at least one substituent group. In embodiments, when R^{3B} is substituted, it is substituted with at least one size-limited substituent group. In embodiments, when R^{3B} is substituted, it is substituted with at least one lower substituent group.

In embodiments, a substituted ring formed when R^{3A} and R^{3B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{3A} and R^{3B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{3A} and R^{3B} substituents bonded to the same nitrogen atom are joined is substituted, it is substituted with at least one substituent group. In embodiments, when the substituted ring formed when R^{3A} and R^{3B} substituents bonded to the same nitrogen atom are joined is substituted, it is substituted with at least one size-limited substituent group. In embodiments, when the substituted ring formed when R^{3A} and R^{3B} substituents bonded to the same nitrogen atom are joined is substituted, it is substituted with at least one lower substituent group.

In embodiments, a substituted R^{3C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3C} is substituted with a plurality of groups selected from substituent groups, size-limited substituent groups, and lower substituent groups; each substituent group, size-limited substituent group, and/or lower substituent group may optionally be different. In embodiments, when R^{3C} is substituted, it is substituted with at least one substituent group. In embodiments, when R^{3C} is substituted, it is substituted with at least one size-limited substituent group. In embodiments, when R^{3C} is substituted, it is substituted with at least one lower substituent group.

In embodiments, a substituted R^{3D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3D} is substituted with a plurality of groups selected from substituent groups, size-limited substituent groups, and lower substituent groups; each substituent group, size-limited substituent group, and/or lower substituent group may optionally be different. In embodiments, when R^{3D} is substituted, it is substituted with at least one substituent group. In embodiments, when R^{3D} is substituted, it is substituted with at least one size-limited substituent group. In embodiments, when R^{3D} is substituted, it is substituted with at least one lower substituent group.

In embodiments, R³ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R³ is hydrogen or halogen. In embodiments, R³ is hydrogen. In embodiments, R³ is halogen.

In embodiments, R⁴ is hydrogen or unsubstituted methyl. In embodiments, R⁴ is hydrogen. In embodiments, R⁴ is unsubstituted methyl. In embodiments, R⁴ is unsubstituted ethyl. In embodiments, R⁴ is unsubstituted propyl. In embodiments, R⁴ is unsubstituted n-propyl. In embodiments, R⁴ is unsubstituted isopropyl. In embodiments, R⁴ is unsubstituted butyl. In embodiments, R⁴ is unsubstituted n-butyl. In embodiments, R⁴ is unsubstituted isobutyl. In embodiments, R⁴ is unsubstituted tert-butyl.

In embodiments, a substituted L^{2A} (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L^{2A} is substituted with a plurality of groups selected from substituent groups, size-limited substituent groups, and lower substituent groups; each substituent group, size-limited substituent group, and/or lower substituent group may optionally be different. In embodiments, when L^{2A} is substituted, it is substituted with at least one substituent group. In embodiments, when L^{2A} is substituted, it is substituted with at least one size-limited substituent group. In embodiments, when L^{2A} is substituted, it is substituted with at least one lower substituent group.

In embodiments, L^{2A} is unsubstituted C₁-C₆ alkylene. In embodiments, L^{2A} is unsubstituted methylene. In embodiments, L^{2A} is unsubstituted ethylene. In embodiments, L^{2A} is unsubstituted propylene. In embodiments, L^{2A} is unsubstituted n-propylene. In embodiments, L^{2A} is unsubstituted butylene. In embodiments, L^{2A} is unsubstituted n-butylene. In embodiments, L^{2A} is unsubstituted pentylene. In embodiments, L^{2A} is unsubstituted n-pentylene. In embodiments, L^{2A} is unsubstituted hexylene. In embodiments, L^{2A} is unsubstituted n-hexylene. In embodiments, L^{2A} is unsubstituted C₁-C₆ alkenylene.

In embodiments, a substituted L^{2B} (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L^{2B} is substituted with a plurality of groups selected from substituent groups, size-limited substituent groups, and lower substituent groups; each substituent group, size-limited substituent group, and/or lower substituent group may optionally be different. In embodiments, when L^{2B} is substituted, it is substituted with at least one substituent group. In embodiments, when L^{2B} is substituted, it is substituted with at least one size-limited substituent group. In embodiments, when L^{2B} is substituted, it is substituted with at least one lower substituent group.

In embodiments, L^{2B} is a bond, —O—, —NH—, unsubstituted C₁-C₆ alkylene, unsubstituted C₃-C₆ cycloalkylene, or unsubstituted 3 to 6 membered heterocycloalkylene. In embodiments, L^{2B} is a bond. In embodiments, L^{2B} is —O—. In embodiments, L^{2B} is —NH—. In embodiments, L^{2B} is unsubstituted methylene. In embodiments, L^{2B} is unsubstituted ethylene. In embodiments, L^{2B} is unsubstituted propylene. In embodiments, L^{2B} is unsubstituted n-propylene. In embodiments, L^{2B} is unsubstituted butylene. In embodiments, L^{2B} is unsubstituted n-butylene. In embodiments, L^{2B} is unsubstituted pentylene. In embodiments, L^{2B} is unsubstituted n-pentylene. In embodiments, L^{2B} is unsubstituted hexylene. In embodiments, L^{2B} is unsubstituted n-hexylene. In embodiments, L^{2B} is unsubstituted C₃-C₆ cycloalkylene. In embodiments, L^{2B} is unsubstituted cyclopropylene. In embodiments, L^{2B} is unsubstituted cyclobutylene. In embodiments, L^{2B} is unsubstituted cyclopentylene. In embodiments, L^{2B} is unsubstituted cyclohexylene. In embodiments, L^{2B} is unsubstituted 3 to 6 membered heterocycloalkylene. In embodiments, L^{2B} is unsubstituted azetidinylene. In embodiments, L^{2B} is unsubstituted pyrrolidinylene.

In embodiments, a substituted L^{2C} (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L^{2C} is substituted with a plurality of groups selected from substituent groups, size-limited substituent groups, and lower substituent groups; each substituent group, size-limited substituent group, and/or lower substituent group may optionally be different. In embodiments, when L^{2C} is substituted, it is substituted with at least one substituent group. In embodiments, when L^{2C} is substituted, it is substituted with at least one size-limited substituent group. In embodiments, when L^{2C} is substituted, it is substituted with at least one lower substituent group.

In embodiments, L^{2C} is a bond, —O—, —NH—, unsubstituted C₁-C₆ alkylene, unsubstituted phenylene, or unsubstituted 5 to 6 membered heteroarylene. In embodiments, L^{2C} is a bond. In embodiments, L^{2C} is —O—. In embodiments, L^{2C} is —NH—. In embodiments, L^{2C} is unsubstituted C₁-C₆ alkylene. In embodiments, L^{2C} is unsubstituted methylene. In embodiments, L^{2C} is unsubstituted ethylene. In embodiments, L^{2C} is unsubstituted propylene. In embodiments, L^{2C} is unsubstituted n-propylene. In embodiments, L^{2C} is unsubstituted butylene. In embodiments, L^{2C} is unsubstituted n-butylene. In embodiments, L^{2C} is unsubstituted pentylene. In embodiments, L^{2C} is unsubstituted n-pentylene. In embodiments, L^{2C} is unsubstituted hexylene. In embodiments, L^{2C} is unsubstituted n-hexylene. In embodiments, L^{2C} is unsubstituted phenylene. In embodiments, L^{2C} is unsubstituted 5 to 6 membered heteroarylene. In embodiments, L^{2C} is unsubstituted pyrazolylene. In embodiments, L^{2C} is unsubstituted imidazolylene. In embodiments, L^{2C} is unsubstituted thiophenylene.

In embodiments, L² is

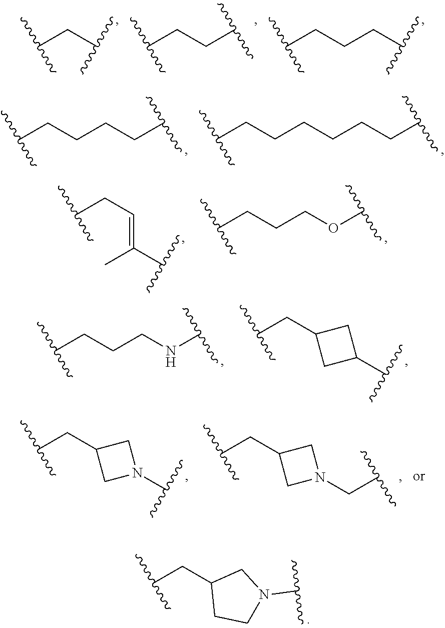

In embodiments, L² is

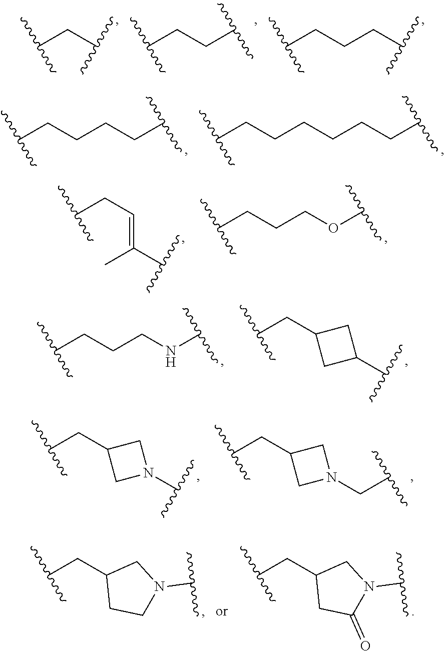

In embodiments, L² is

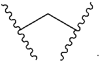 [SMILES: *C*]

In embodiments, L² is

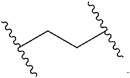 [SMILES: *CC*]

In embodiments, L² is

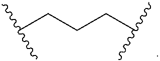 [SMILES: *CCC*]

In embodiments, L² is

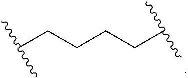 [SMILES: *CCCC*]

In embodiments, L² is

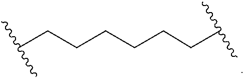 [SMILES: *CCCCCC*]

In embodiments, L² is

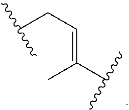 [SMILES: *C/C=C(/*)C]

In embodiments, L² is

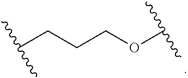 [SMILES: *CCCO*]

In embodiments, L² is

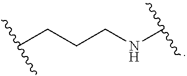 [SMILES: *CCCN*]

In embodiments, L² is

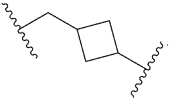 [SMILES: *CC1CC(*)C1]

In embodiments, L² is

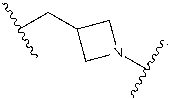 [SMILES: *CC1CN(*)C1]

In embodiments, L² is

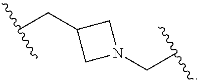 [SMILES: *CC1CN(C*)C1]

In embodiments, L² is

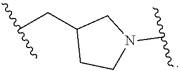 [SMILES: *CC1CCN(*)C1]

In embodiments, L² is

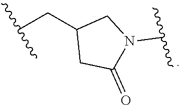 [SMILES: *CC1CC(=O)N(*)C1]

In embodiments, a substituted R⁵ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁵ is substituted with a plurality of groups selected from substituent groups, size-limited substituent groups, and lower substituent groups; each substituent group, size-limited substituent group, and/or lower substituent group may optionally be different. In embodiments, when R⁵ is substituted, it is substituted with at least one substituent group. In embodiments, when R⁵ is substituted, it is substituted with at least one size-limited substituent group. In embodiments, when R⁵ is substituted, it is substituted with at least one lower substituent group.

In embodiments, a substituted R^{5A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{5A} is substituted with a plurality of groups selected from substituent groups, size-limited substituent groups, and lower substituent groups; each substituent group, size-limited substituent group, and/or lower substituent group may optionally be different. In embodiments, when R^{5A} is substituted, it is substituted with at least one substituent group. In embodiments, when R^{5A} is substituted, it is substituted with at least one size-limited substituent group. In embodiments, when R^{5A} is substituted, it is substituted with at least one lower substituent group.

In embodiments, a substituted R^{5B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{5B} is substituted with a plurality of groups selected from substituent groups, size-limited substituent groups, and lower substituent groups; each substituent group, size-limited substituent group, and/or lower substituent group may optionally be different. In embodiments, when R^{5B} is substituted, it is substituted with at least one substituent group. In embodiments, when R^{5B} is substituted, it is substituted with at least one size-limited substituent group. In embodiments, when R^{5B} is substituted, it is substituted with at least one lower substituent group.

In embodiments, a substituted ring formed when R^{5A} and R^{5B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{5A} and R^{5B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{5A} and R^{5B} substituents bonded to the same nitrogen atom are joined is substituted, it is substituted with at least one substituent group. In embodiments, when the substituted ring formed when R^{5A} and R^{5B} substituents bonded to the same nitrogen atom are joined is substituted, it is substituted with at least one size-limited substituent group. In embodiments, when the substituted ring formed when R^{5A} and R^{5B} substituents bonded to the same nitrogen atom are joined is substituted, it is substituted with at least one lower substituent group.

In embodiments, a substituted R^{5C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{5C} is substituted with a plurality of groups selected from substituent groups, size-limited substituent groups, and lower substituent groups; each substituent group, size-limited substituent group, and/or lower substituent group may optionally be different. In embodiments, when R^{5C} is substituted, it is substituted with at least one substituent group. In embodiments, when R^{5C} is substituted, it is substituted with at least one size-limited substituent group. In embodiments, when R^{5C} is substituted, it is substituted with at least one lower substituent group.

In embodiments, a substituted R^{5D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{5D} is substituted with a plurality of groups selected from substituent groups, size-limited substituent groups, and lower substituent groups; each substituent group, size-limited substituent group, and/or lower substituent group may optionally be different. In embodiments, when R^{5D} is substituted, it is substituted with at least one substituent group. In embodiments, when R^{5D} is substituted, it is substituted with at least one size-limited substituent group. In embodiments, when R^{5D} is substituted, it is substituted with at least one lower substituent group.

In embodiments, R⁵ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R⁵ is hydrogen, halogen, —CF₃, —CHF₂, —CH₂F, —OCF₃, —OCHF₂, —OCH₂F, —CN, —SF₅, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl. In embodiments, R⁵ is hydrogen. In embodiments, R⁵ is halogen. In embodiments, R⁵ is —CF₃. In embodiments, R⁵ is —CHF₂. In embodiments, R⁵ is —CH₂F. In embodiments, R⁵ is —OCF₃. In embodiments, R⁵ is —OCHF₂. In embodiments, R⁵ is —OCH₂F. In embodiments, R⁵ is —CN. In embodiments, R⁵ is —SF₅. In embodiments, R⁵ is hydrogen or unsubstituted C₁-C₆ alkyl. In embodiments, R⁵ is unsubstituted methyl. In embodiments, R⁵ is unsubstituted ethyl. In embodiments, R⁵ is unsubstituted propyl. In embodiments, R⁵ is unsubstituted n-propyl. In embodiments, R⁵ is unsubstituted isopropyl. In embodiments, R⁵ is unsubstituted butyl. In embodiments, R⁵ is unsubstituted n-butyl. In embodiments, R⁵ is unsubstituted isobutyl. In embodiments, R⁵ is unsubstituted tert-butyl. In embodiments, R⁵ is unsubstituted pentyl. In embodiments, R⁵ is unsubstituted hexyl. In embodiments, R⁵ is substituted or unsubstituted 2 to 6 membered heteroalkyl. In embodiments, R⁵ is substituted or unsubstituted C₃-C₆ cycloalkyl. In embodiments, R⁵ is substituted or unsubstituted cyclopropyl. In embodiments, R⁵ is substituted or unsubstituted cyclobutyl. In embodiments, R⁵ is substituted or unsubstituted cyclopentyl. In embodiments, R⁵ is substituted or unsubstituted cyclohexyl. In embodiments, R⁵ is substituted or unsubstituted 3 to 6 membered heterocycloalkyl. In embodiments, R⁵ is substituted or unsubstituted tetrahydropyranyl. In embodiments, R⁵ is substituted or unsubstituted piperidinyl. In embodiments, R⁵ is substituted or unsubstituted phenyl. In embodiments, R⁵ is substituted or unsubstituted 5 to 6 membered heteroaryl. In embodiments, R⁵ is substituted or unsubstituted isoxazolyl. In embodiments, R⁵ is substituted or unsubstituted pyrazolyl. In embodiments, R⁵ is substituted or unsubstituted imidazolyl. In embodiments, R⁵ is substituted or unsubstituted thiophenyl. In embodiments, R⁵ is substituted or unsubstituted thiazolyl. In embodiments, R⁵ is substituted or unsubstituted pyridyl. In embodiments, R⁵ is substituted or unsubstituted benzimidazolyl. In embodiments, R⁵ is substituted or unsubstituted 2,3-dihydrobenzofuranyl.

In embodiments, R⁵ is hydrogen, —CF₃, —CHF₂, —OCF₃,

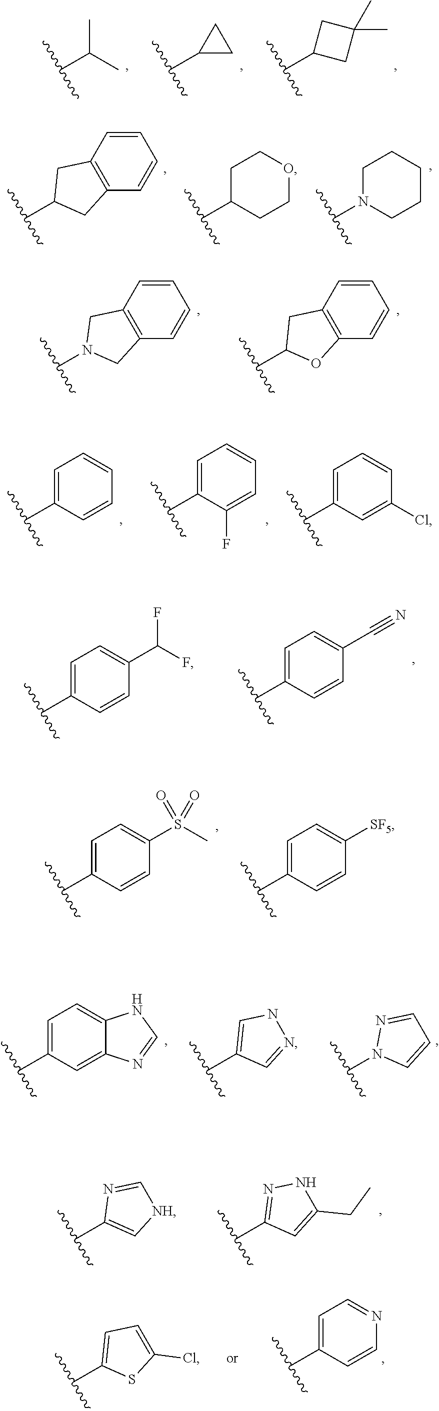

In embodiments, R⁵ is hydrogen, —CF₃, —CHF₂, —OCF₃,

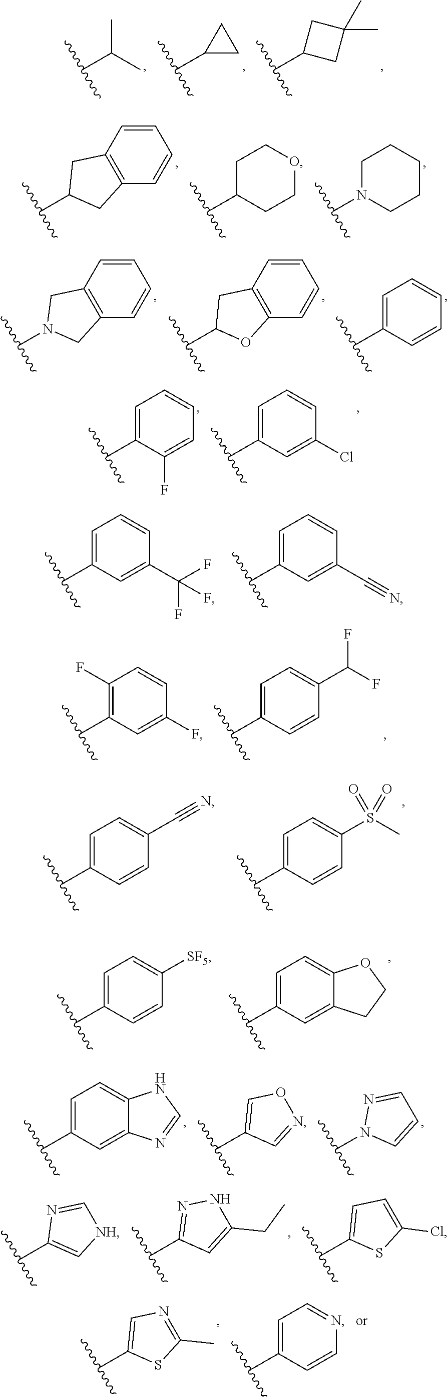

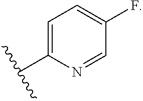 [SMILES: *c1ccc(F)cn1]

In embodiments, R⁵ is hydrogen. In embodiments, R⁵ is —CF₃. In embodiments, R⁵ is —CHF₂. In embodiments, R⁵ is —OCF₃. In embodiments, R⁵ is

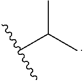 [SMILES: *C(C)C]

In embodiments, R⁵ is

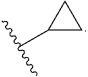 [SMILES: *C1CC1]

In embodiments, R⁵ is

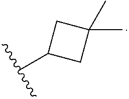 [SMILES: *C1CC(C)(C)C1]

In embodiments, R⁵ is

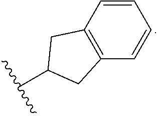 [SMILES: *C1Cc2ccccc2C1]

In embodiments, R⁵ is

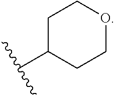 [SMILES: *C1CCOCC1]

In embodiments, R⁵ is

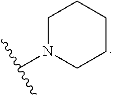 [SMILES: *N1CCCCC1]

In embodiments, R⁵ is

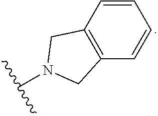 [SMILES: *N1Cc2ccccc2C1]

In embodiments, R⁵ is

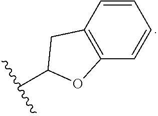 [SMILES: *C1Cc2ccccc2O1]

In embodiments, R⁵ is

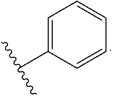 [SMILES: *c1ccccc1]

In embodiments, R⁵ is

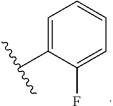 [SMILES: *c1ccccc1F]

In embodiments, R⁵ is

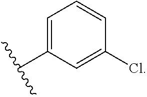 [SMILES: *c1cccc(Cl)c1]

In embodiments, R⁵ is

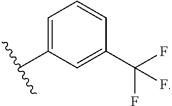 [SMILES: *c1cccc(C(F)(F)F)c1]

In embodiments, R⁵ is

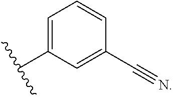 [SMILES: *c1cccc(C#N)c1]

In embodiments, R⁵ is

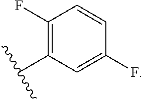 [SMILES: *c1cc(F)ccc1F]

In embodiments, R⁵ is

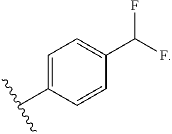 [SMILES: *c1ccc(C(F)F)cc1]

In embodiments, R⁵ is

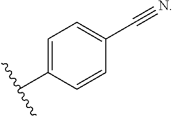 [SMILES: *c1ccc(C#N)cc1]

In embodiments, R⁵ is

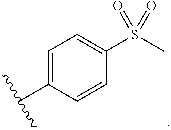 [SMILES: *c1ccc(S(C)(=O)=O)cc1]

In embodiments, R⁵ is

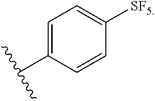

In embodiments, R⁵ is

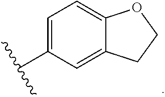 [SMILES: *c1ccc2c(c1)CCO2]

In embodiments, R⁵ is

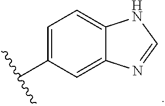 [SMILES: *c1ccc2[nH]cnc2c1]

In embodiments, R⁵ is

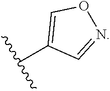 [SMILES: *c1cnoc1]

In embodiments, R⁵ is

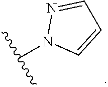 [SMILES: *n1cccn1]

In embodiments, R⁵ is

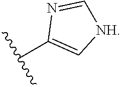 [SMILES: *c1c[nH]cn1]

In embodiments, R⁵ is

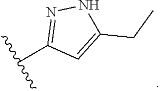 [SMILES: *c1cc(CC)[nH]n1]

In embodiments, R⁵ is

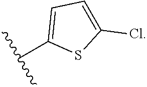 [SMILES: *c1ccc(Cl)s1]

In embodiments, R⁵ is

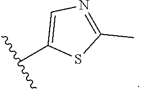 [SMILES: *c1cnc(C)s1]

In embodiments, R⁵ is

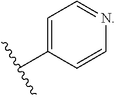 [SMILES: *c1ccncc1]

In embodiments, R⁵ is

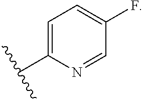 [SMILES: *c1ccc(F)cn1]

In embodiments, -L²-R⁵ is

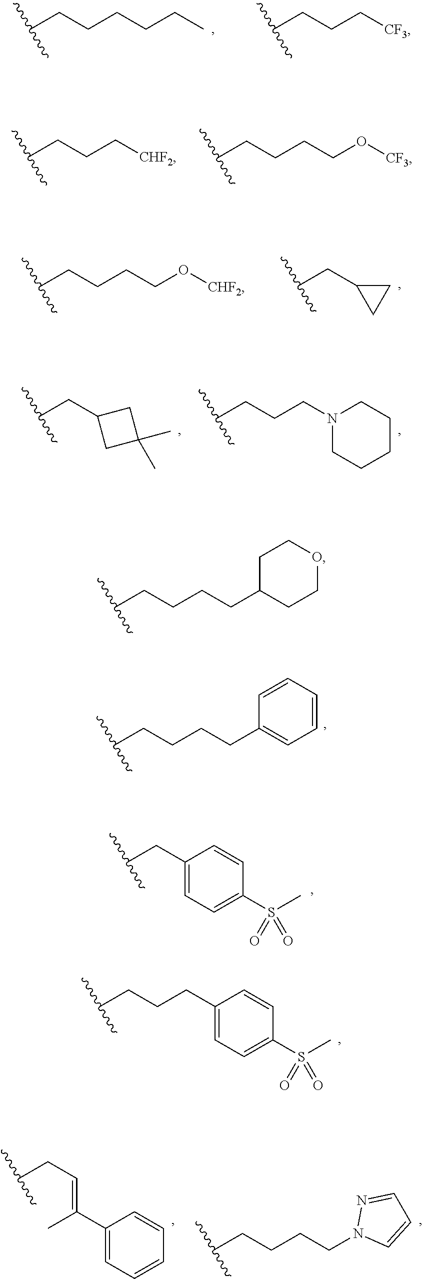

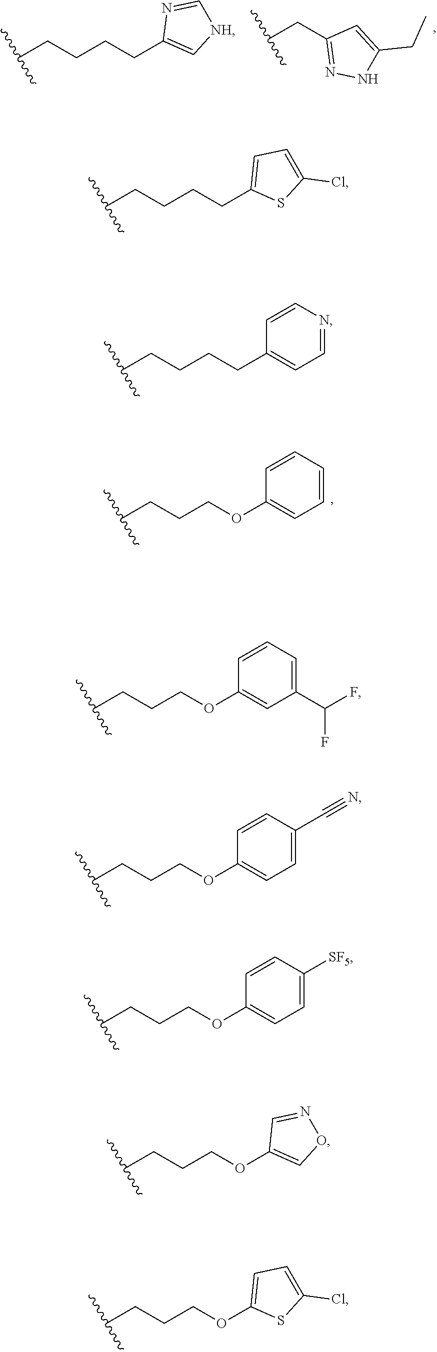

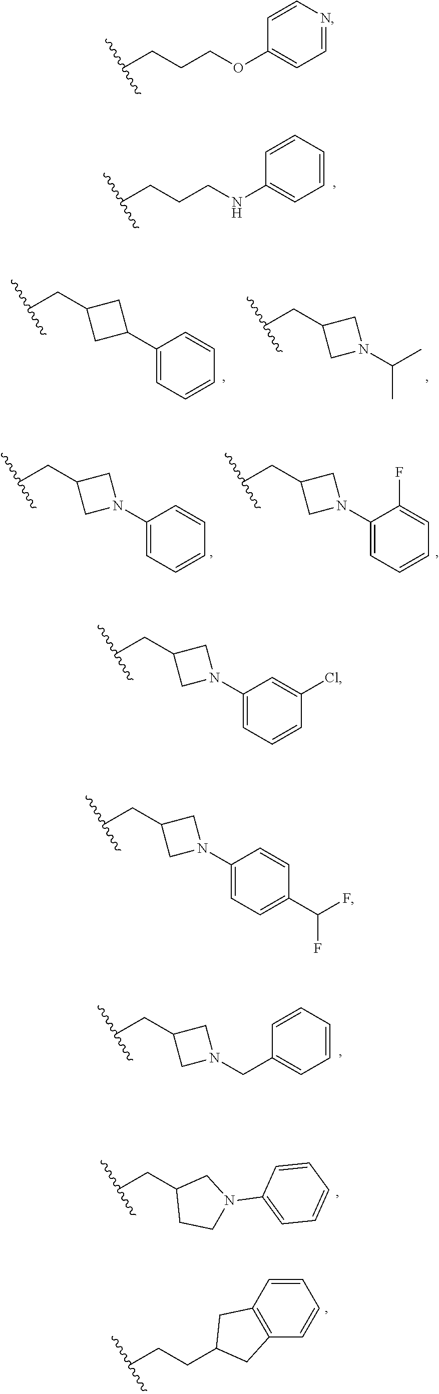

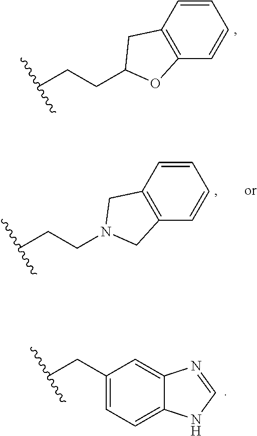 [SMILES: *CCC1Cc2ccccc2O1 ; *CCN1Cc2ccccc2C1 ; *Cc1ccc2[nH]cnc2c1]

In embodiments, -L²-R⁵ is

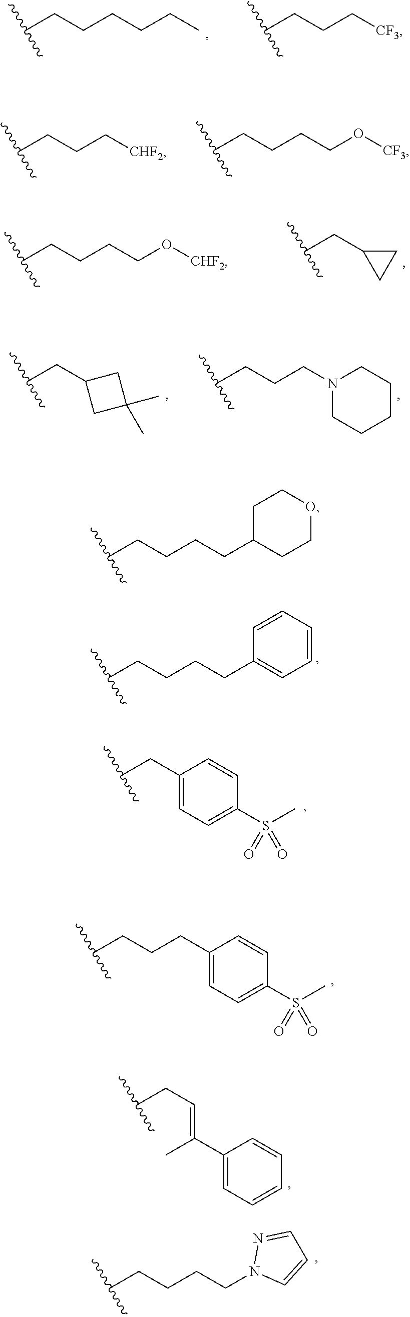

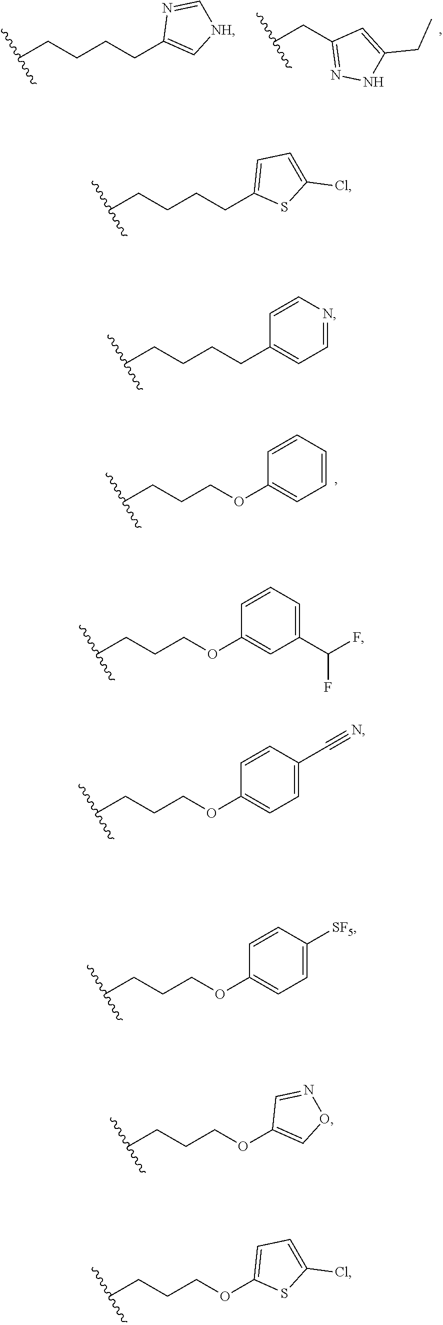

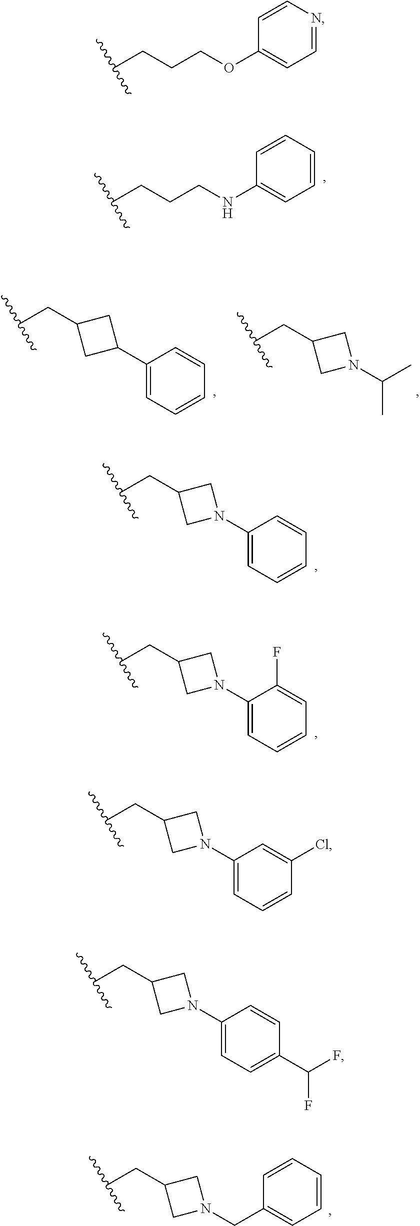

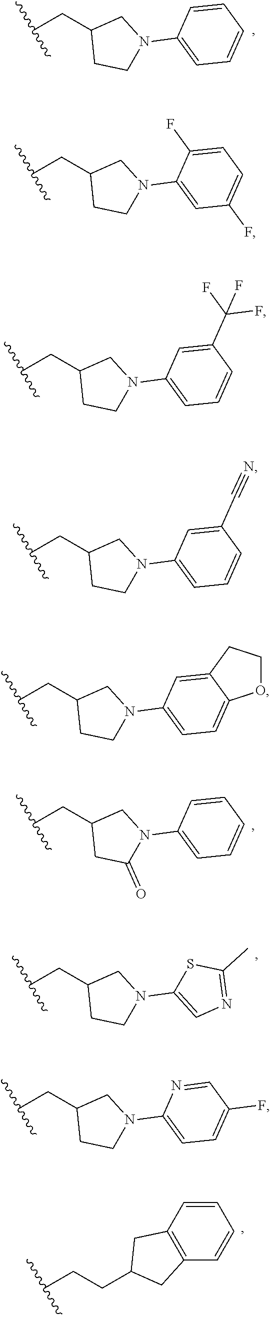

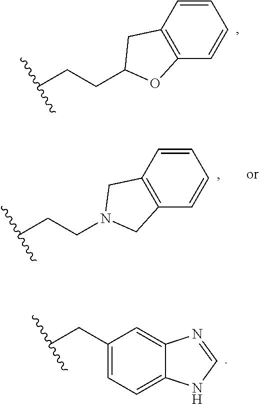 [SMILES: *CCC1Cc2ccccc2O1 ; *CCN1Cc2ccccc2C1 ; *Cc1ccc2[nH]cnc2c1]

In embodiments, -L²-R⁵ is

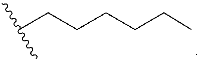 [SMILES: *CCCCCC]

In embodiments, -L²-R⁵ is

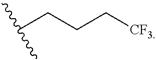 [SMILES: *CCCC(F)(F)F]

In embodiments, -L²-R⁵ is

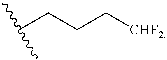 [SMILES: *CCCC(F)F]

In embodiments, -L²-R⁵ is

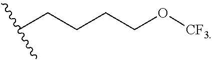 [SMILES: *CCCCOC(F)(F)F]

In embodiments, -L²-R⁵ is

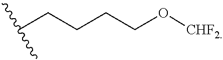 [SMILES: *CCCCOC(F)F]

In embodiments, -L²-R⁵ is

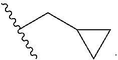 [SMILES: *CC1CC1]

In embodiments, -L²-R⁵ is

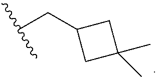 [SMILES: *CC1CC(C)(C)C1]

In embodiments, -L²-R⁵ is

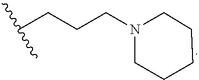 [SMILES: *CCCN1CCCCC1]

In embodiments, -L²-R⁵ is

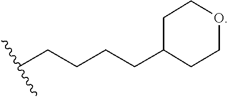 [SMILES: *CCCCC1CCOCC1]

In embodiments, -L²-R⁵ is

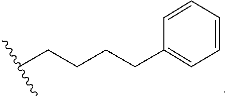 [SMILES: *CCCCc1ccccc1]

In embodiments, -L²-R⁵ is

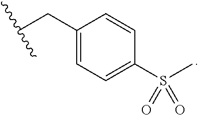 [SMILES: *Cc1ccc(S(C)(=O)=O)cc1]

In embodiments, -L²-R⁵ is

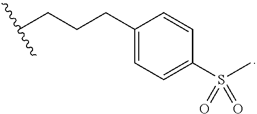 [SMILES: *CCCc1ccc(S(C)(=O)=O)cc1]

In embodiments, -L²-R⁵ is

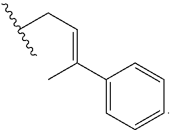 [SMILES: *C/C=C(\C)c1ccccc1]

In embodiments, -L²-R⁵ is

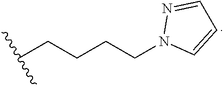 [SMILES: *CCCCn1cccn1]

In embodiments, -L²-R⁵ is

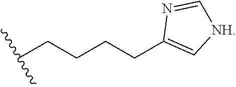 [SMILES: *CCCCc1c[nH]cn1]

In embodiments, -L²-R⁵ is

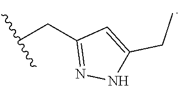 [SMILES: *Cc1cc(CC)[nH]n1]

In embodiments, -L²-R⁵ is

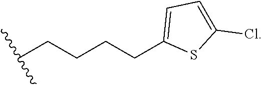 [SMILES: *CCCCc1ccc(Cl)s1]

In embodiments, -L²-R⁵ is

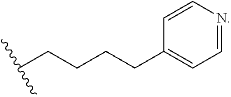 [SMILES: *CCCCc1ccncc1]

In embodiments, -L²-R⁵ is

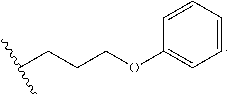 [SMILES: *CCCOc1ccccc1]

In embodiments, -L²-R⁵ is

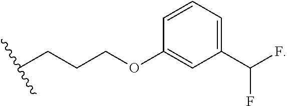 [SMILES: *CCCOc1cccc(C(F)F)c1]

In embodiments, -L²-R⁵ is

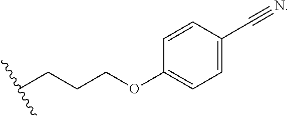 [SMILES: *CCCOc1ccc(C#N)cc1]

In embodiments, -L²-R⁵ is

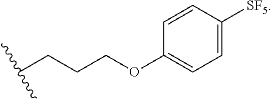

In embodiments, -L²-R⁵ is

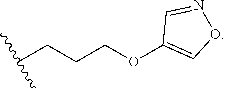 [SMILES: *CCCOc1cnoc1]

In embodiments, -L²-R⁵ is

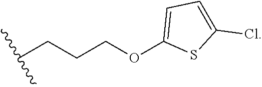 [SMILES: *CCCOc1ccc(Cl)s1]

In embodiments, -L²-R⁵ is

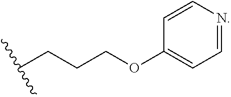 [SMILES: *CCCOc1ccncc1]

### In

embodiments, -L²-R⁵ is

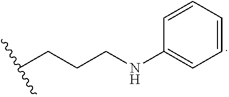 [SMILES: *CCCNc1ccccc1]

In embodiments, -L²-R⁵ is

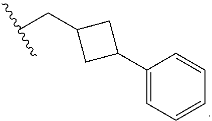 [SMILES: *CC1CC(c2ccccc2)C1]

In embodiments, -L²-R⁵ is

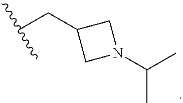 [SMILES: *CC1CN(C(C)C)C1]

In embodiments, -L²-R⁵ is

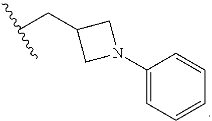 [SMILES: *CC1CN(c2ccccc2)C1]

In embodiments, -L²-R⁵ is

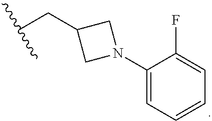 [SMILES: *CC1CN(c2ccccc2F)C1]

In embodiments, -L²-R⁵ is

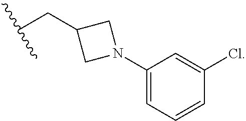 [SMILES: *CC1CN(c2cccc(Cl)c2)C1]

In embodiments, -L²-R⁵ is

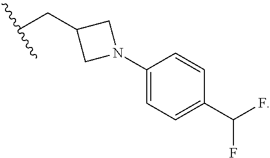 [SMILES: *CC1CN(c2ccc(C(F)F)cc2)C1]

In embodiments, -L²-R⁵ is

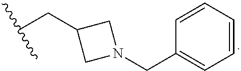 [SMILES: *CC1CN(Cc2ccccc2)C1]

In embodiments, -L²-R⁵ is

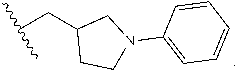 [SMILES: *CC1CCN(c2ccccc2)C1]

In embodiments, -L²-R⁵ is

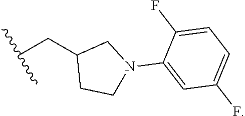 [SMILES: *CC1CCN(c2cc(F)ccc2F)C1]

In embodiments, -L²-R⁵ is

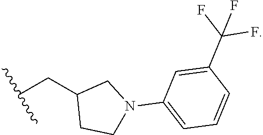 [SMILES: *CC1CCN(c2cccc(C(F)(F)F)c2)C1]

In embodiments, -L²-R⁵ is

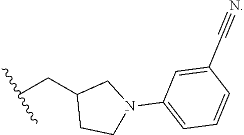 [SMILES: *CC1CCN(c2cccc(C#N)c2)C1]

In embodiments, -L²-R⁵ is

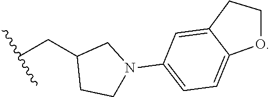 [SMILES: *CC1CCN(c2ccc3c(c2)CCO3)C1]

In embodiments, -L²-R⁵ is

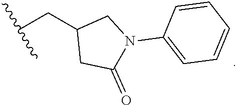 [SMILES: *CC1CC(=O)N(c2ccccc2)C1]

In embodiments, -L²-R⁵ is

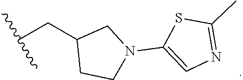 [SMILES: *CC1CCN(c2cnc(C)s2)C1]

In embodiments, -L²-R⁵ is

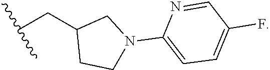 [SMILES: *CC1CCN(c2ccc(F)cn2)C1]

In embodiments, -L²-R⁵ is

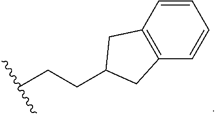 [SMILES: *CCC1Cc2ccccc2C1]

In embodiments, -L²-R⁵ is

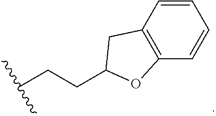 [SMILES: *CCC1Cc2ccccc2O1]

In embodiments, -L²-R⁵ is

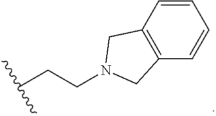 [SMILES: *CCN1Cc2ccccc2C1]

In embodiments, -L²-R⁵ is

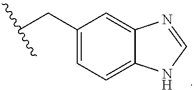 [SMILES: *Cc1ccc2[nH]cnc2c1]

In embodiments, when Ring A is substituted, Ring A is substituted with one or more first substituent groups denoted by R^{A.1} as explained in the definitions section above in the description of “first substituent group(s)”. In embodiments, when an R^{A.1} substituent group is substituted, the R^{A.1} substituent group is substituted with one or more second substituent groups denoted by R^{A.2} as explained in the definitions section above in the description of “first substituent group(s)”. In embodiments, when an R^{A.2} substituent group is substituted, the R^{A.2} substituent group is substituted with one or more third substituent groups denoted by R^{A.3} as explained in the definitions section above in the description of “first substituent group(s)”. In the above embodiments, R^{A}, R^{A.1}, R^{A.2}, and R^{A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{A}, R^{A.1}, R^{A.2}, and R^{A.3}, respectively.

In embodiments, when R¹ is substituted, R¹ is substituted with one or more first substituent groups denoted by R^{1.1} as explained in the definitions section above in the description of “first substituent group(s)”. In embodiments, when an R^{1.1} substituent group is substituted, the R^{1.1} substituent group is substituted with one or more second substituent groups denoted by R^{1.2} as explained in the definitions section above in the description of “first substituent group(s)”. In embodiments, when an R^{1.2} substituent group is substituted, the R^{1.2} substituent group is substituted with one or more third substituent groups denoted by R^{1.3} as explained in the definitions section above in the description of “first substituent group(s)”. In the above embodiments, R¹, R^{1.1}, R^{1.2}, and R^{1.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹, R^{1.1}, R^{1.2}, and R^{1.3}, respectively.

In embodiments, when R^{1A} is substituted, R^{1A} is substituted with one or more first substituent groups denoted by R^{1A.1} as explained in the definitions section above in the description of “first substituent group(s)”. In embodiments, when an R^{1A.1} substituent group is substituted, the R^{1A.1} substituent group is substituted with one or more second substituent groups denoted by R^{1A.2} as explained in the definitions section above in the description of “first substituent group(s)”. In embodiments, when an R^{1A.2} substituent group is substituted, the R^{1A.2} substituent group is substituted with one or more third substituent groups denoted by R^{1A.3} as explained in the definitions section above in the description of “first substituent group(s)”. In the above embodiments, R^{1A}, R^{1A.1}, R^{1A.2}, and R^{1A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{1A}, R^{1A.1}, R^{1A.2}, and R^{1A.3}, respectively.

In embodiments, when R^{1B} is substituted, R^{1B} is substituted with one or more first substituent groups denoted by R^{1B.1} as explained in the definitions section above in the description of “first substituent group(s)”. In embodiments, when an R^{1B.1} substituent group is substituted, the R^{1B.1} substituent group is substituted with one or more second substituent groups denoted by R^{1B.2} as explained in the definitions section above in the description of “first substituent group(s)”. In embodiments, when an R^{1B.2} substituent group is substituted, the R^{1B.2} substituent group is substituted with one or more third substituent groups denoted by R^{1B.3} as explained in the definitions section above in the description of “first substituent group(s)”. In the above embodiments, R^{1B}, R^{1B.1}, R^{1B.2}, and R^{1B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{1B}, R^{1B.1}, R^{1B.2}, and R^{1B.3}, respectively.

In embodiments, when R^{1A} and R^{1B} substituents bonded to the same nitrogen atom are optionally joined to form a moiety that is substituted (e.g., a substituted heterocycloalkyl or substituted heteroaryl), the moiety is substituted with one or more first substituent groups denoted by R^{1A.1} as explained in the definitions section above in the description of “first substituent group(s)”. In embodiments, when an R^{1A.1} substituent group is substituted, the R^{1A.1} substituent group is substituted with one or more second substituent groups denoted by R^{1A.2} as explained in the definitions section above in the description of “first substituent group(s)”. In embodiments, when an R^{1A.2} substituent group is substituted, the R^{1A.2} substituent group is substituted with one or more third substituent groups denoted by R^{1A.3} as explained in the definitions section above in the description of “first substituent group(s)”. In the above embodiments, R^{1A.1}, R^{1A.2}, and R^{1A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{1A.1}, R^{1A.2}, and R^{1A.3}, respectively.

In embodiments, when R^{1A} and R^{1B} substituents bonded to the same nitrogen atom are optionally joined to form a moiety that is substituted (e.g., a substituted heterocycloalkyl or substituted heteroaryl), the moiety is substituted with one or more first substituent groups denoted by R^{1B.1} as explained in the definitions section above in the description of “first substituent group(s)”. In embodiments, when an R^{1B.1} substituent group is substituted, the R^{1B.1} substituent group is substituted with one or more second substituent groups denoted by R^{1B.2} as explained in the definitions section above in the description of “first substituent group(s)”. In embodiments, when an R^{1B.2} substituent group is substituted, the R^{1B.2} substituent group is substituted with one or more third substituent groups denoted by R^{1B.3} as explained in the definitions section above in the description of “first substituent group(s)”. In the above embodiments, R^{1B.1}, R^{1B.2}, and R^{1B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{1B.1}, R^{1B.2}, and R^{1B.3}, respectively.

In embodiments, when R^{1C} is substituted, R^{1C} is substituted with one or more first substituent groups denoted by R^{1C.1} as explained in the definitions section above in the description of “first substituent group(s)”. In embodiments, when an R^{1C.1} substituent group is substituted, the R^{1C.1} substituent group is substituted with one or more second substituent groups denoted by R^{1C.2} as explained in the definitions section above in the description of “first substituent group(s)”. In embodiments, when an R^{1C.2} substituent group is substituted, the R^{1C.2} substituent group is substituted with one or more third substituent groups denoted by R^{1C.3} as explained in the definitions section above in the description of “first substituent group(s)”. In the above embodiments, R^{1C}, R^{1C.1}, R^{1C.2}, and R^{1C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{1C}, R^{1C.1}, R^{1C.2}, and R^{1C.3}, respectively.

In embodiments, when R^{1D} is substituted, R^{1D} is substituted with one or more first substituent groups denoted by R^{1D.1} as explained in the definitions section above in the description of “first substituent group(s)”. In embodiments, when an R^{1D.1} substituent group is substituted, the R^{1D.1} substituent group is substituted with one or more second substituent groups denoted by R^{1D.2} as explained in the definitions section above in the description of “first substituent group(s)”. In embodiments, when an R^{1D.2} substituent group is substituted, the R^{1D.2} substituent group is substituted with one or more third substituent groups denoted by R^{1D.3} as explained in the definitions section above in the description of “first substituent group(s)”. In the above embodiments, R^{1D}, R^{1D.1}, R^{1D.2}, and R^{1D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{1D}, R^{1D.1}, R^{1D.2}, and R^{1D.3}, respectively.

In embodiments, when R² is substituted, R² is substituted with one or more first substituent groups denoted by R^{2.1} as explained in the definitions section above in the description of “first substituent group(s)”. In embodiments, when an R^{2.1} substituent group is substituted, the R^{2.1} substituent group is substituted with one or more second substituent groups denoted by R^{2.2} as explained in the definitions section above in the description of “first substituent group(s)”. In embodiments, when an R^{2.2} substituent group is substituted, the R^{2.2} substituent group is substituted with one or more third substituent groups denoted by R^{2.3} as explained in the definitions section above in the description of “first substituent group(s)”. In the above embodiments, R², R^{2.1}, R^{2.2}, and R^{2.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R², R^{2.1}, R^{2.2}, and R^{2.3}, respectively.

In embodiments, when R^{2A} is substituted, R^{2A} is substituted with one or more first substituent groups denoted by R^{2A.1} as explained in the definitions section above in the description of “first substituent group(s)”. In embodiments, when an R^{2A.1} substituent group is substituted, the R^{2A.1} substituent group is substituted with one or more second substituent groups denoted by R^{2A.2} as explained in the definitions section above in the description of “first substituent group(s)”. In embodiments, when an R^{2A.2} substituent group is substituted, the R^{2A.2} substituent group is substituted with one or more third substituent groups denoted by R^{2A.3} as explained in the definitions section above in the description of “first substituent group(s)”. In the above embodiments, R^{2A}, R^{2A.1}, R^{2A.2}, and R^{2A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{2A}, R^{2A.1}, R^{2A.2}, and R^{2A.3}, respectively.

In embodiments, when R^{2B} is substituted, R^{2B} is substituted with one or more first substituent groups denoted by R^{2B.1} as explained in the definitions section above in the description of “first substituent group(s)”. In embodiments, when an R^{2B.1} substituent group is substituted, the R^{2B} substituent group is substituted with one or more second substituent groups denoted by R^{2B.2} as explained in the definitions section above in the description of “first substituent group(s)”. In embodiments, when an R^{2B.2} substituent group is substituted, the R^{2B.2} substituent group is substituted with one or more third substituent groups denoted by R^{2B.3} as explained in the definitions section above in the description of “first substituent group(s)”. In the above embodiments, R^{2B}, R^{2B.1}, R^{2B.2}, and R^{2B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{2B}, R^{2B.1}, R^{2B.2}, and R^{2B.3}, respectively.

In embodiments, when R^{2A} and R^{2B} substituents bonded to the same nitrogen atom are optionally joined to form a moiety that is substituted (e.g., a substituted heterocycloalkyl or substituted heteroaryl), the moiety is substituted with one or more first substituent groups denoted by R^{2A.1} as explained in the definitions section above in the description of “first substituent group(s)”. In embodiments, when an R^{2A.1} substituent group is substituted, the R^{2A.1} substituent group is substituted with one or more second substituent groups denoted by R^{2A.2} as explained in the definitions section above in the description of “first substituent group(s)”. In embodiments, when an R^{2A.2} substituent group is substituted, the R^{2A.2} substituent group is substituted with one or more third substituent groups denoted by R^{2A.3} as explained in the definitions section above in the description of “first substituent group(s)”. In the above embodiments, R^{2A}0.1, R^{2A.2}, and R^{2A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{2A.1}, R^{2A.2}, and R^{2A.3}, respectively.

In embodiments, when R^{2A} and R^{2B} substituents bonded to the same nitrogen atom are optionally joined to form a moiety that is substituted (e.g., a substituted heterocycloalkyl or substituted heteroaryl), the moiety is substituted with one or more first substituent groups denoted by R^{2B.1} as explained in the definitions section above in the description of “first substituent group(s)”. In embodiments, when an R^{2B.1} substituent group is substituted, the R^{2B.1} substituent group is substituted with one or more second substituent groups denoted by R^{2B.2} as explained in the definitions section above in the description of “first substituent group(s)”. In embodiments, when an R^{2B.2} substituent group is substituted, the R^{2B.2} substituent group is substituted with one or more third substituent groups denoted by R^{2B.3} as explained in the definitions section above in the description of “first substituent group(s)”. In the above embodiments, R^{2B.1}, R^{2B.2}, and R^{2B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{2B.1}, R^{2B.2}, and R^{2B.3}, respectively.

In embodiments, when R^{2C} is substituted, R^{2C} is substituted with one or more first substituent groups denoted by R^{2C.1} as explained in the definitions section above in the description of “first substituent group(s)”. In embodiments, when an R^{2C.1} substituent group is substituted, the R^{2C.1} substituent group is substituted with one or more second substituent groups denoted by R^{2C.2} as explained in the definitions section above in the description of “first substituent group(s)”. In embodiments, when an R^{2C.2} substituent group is substituted, the R^{2C.2} substituent group is substituted with one or more third substituent groups denoted by R^{2C.3} as explained in the definitions section above in the description of “first substituent group(s)”. In the above embodiments, R^{2C}, R^{2C.1}, R^{2C.2}, and R^{2C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{2C}, R^{2C.1}, R^{2C.2}, and R^{2C.3}, respectively.

In embodiments, when R^{2D} is substituted, R^{2D} is substituted with one or more first substituent groups denoted by R^{2D.1} as explained in the definitions section above in the description of “first substituent group(s)”. In embodiments, when an R^{2D.1} substituent group is substituted, the R^{2D.1} substituent group is substituted with one or more second substituent groups denoted by R^{2D.2} as explained in the definitions section above in the description of “first substituent group(s)”. In embodiments, when an R^{2D.2} substituent group is substituted, the R^{2D.2} substituent group is substituted with one or more third substituent groups denoted by R^{2D.3} as explained in the definitions section above in the description of “first substituent group(s)”. In the above embodiments, R^{2D}, R^{2D.1}, R^{2D.2}, and R^{2D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{2D}, R^{2D.1}, R^{2D.2}, and R^{2D.3}, respectively.

In embodiments, when R³ is substituted, R³ is substituted with one or more first substituent groups denoted by R^{3.1} as explained in the definitions section above in the description of “first substituent group(s)”. In embodiments, when an R^{3.1} substituent group is substituted, the R^{3.1} substituent group is substituted with one or more second substituent groups denoted by R^{3.2} as explained in the definitions section above in the description of “first substituent group(s)”. In embodiments, when an R^{3.2} substituent group is substituted, the R^{3.2} substituent group is substituted with one or more third substituent groups denoted by R^{3.3} as explained in the definitions section above in the description of “first substituent group(s)”. In the above embodiments, R³, R^{3.1}, R^{3.2}, and R^{3.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R³, R^{3.1}, R^{3.2}, and R^{3.3}, respectively.

In embodiments, when R^{3A} is substituted, R^{3A} is substituted with one or more first substituent groups denoted by R^{3A.1} as explained in the definitions section above in the description of “first substituent group(s)”. In embodiments, when an R^{3A.1} substituent group is substituted, the R^{3A.1} substituent group is substituted with one or more second substituent groups denoted by R^{3A.2} as explained in the definitions section above in the description of “first substituent group(s)”. In embodiments, when an R^{3A.2} substituent group is substituted, the R^{3A.2} substituent group is substituted with one or more third substituent groups denoted by R^{3A.3} as explained in the definitions section above in the description of “first substituent group(s)”. In the above embodiments, R^{3A}, R^{3A.1}, R^{3A.2}, and R^{3A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{3A}, R^{3A.1}, R^{3A.2}, and R^{3A.3}, respectively.

In embodiments, when R^{3B} is substituted, R^{3B} is substituted with one or more first substituent groups denoted by R^{3B.1} as explained in the definitions section above in the description of “first substituent group(s)”. In embodiments, when an R^{3B.1} substituent group is substituted, the R^{3B.1} substituent group is substituted with one or more second substituent groups denoted by R^{3B.2} as explained in the definitions section above in the description of “first substituent group(s)”. In embodiments, when an R^{3B.2} substituent group is substituted, the R^{3B.2} substituent group is substituted with one or more third substituent groups denoted by R^{3B.3} as explained in the definitions section above in the description of “first substituent group(s)”. In the above embodiments, R^{3B}, R^{3B.1}, R^{3B.2}, and R^{3B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{3B}, R^{3B.1}, R^{3B.2}, and R^{3B.3}, respectively.

In embodiments, when R^{3A} and R^{3B} substituents bonded to the same nitrogen atom are optionally joined to form a moiety that is substituted (e.g., a substituted heterocycloalkyl or substituted heteroaryl), the moiety is substituted with one or more first substituent groups denoted by R^{3A.1} as explained in the definitions section above in the description of “first substituent group(s)”. In embodiments, when an R^{3A.1} substituent group is substituted, the R^{3A.1} substituent group is substituted with one or more second substituent groups denoted by R^{3A.2} as explained in the definitions section above in the description of “first substituent group(s)”. In embodiments, when an R^{3A.2} substituent group is substituted, the R^{3A.2} substituent group is substituted with one or more third substituent groups denoted by R^{3A.3} as explained in the definitions section above in the description of “first substituent group(s)”. In the above embodiments, R^{3A.1}, R^{3A.2}, and R^{3A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{3A.1}, R^{3A.2}, and R^{3A.3}, respectively.

In embodiments, when R^{3A} and R^{3B} substituents bonded to the same nitrogen atom are optionally joined to form a moiety that is substituted (e.g., a substituted heterocycloalkyl or substituted heteroaryl), the moiety is substituted with one or more first substituent groups denoted by R^{3B.1} as explained in the definitions section above in the description of “first substituent group(s)”. In embodiments, when an R^{3B.1} substituent group is substituted, the R^{3B.1} substituent group is substituted with one or more second substituent groups denoted by R^{3B.2} as explained in the definitions section above in the description of “first substituent group(s)”. In embodiments, when an R^{3B.2} substituent group is substituted, the R^{3B.2} substituent group is substituted with one or more third substituent groups denoted by R^{3B.3} as explained in the definitions section above in the description of “first substituent group(s)”. In the above embodiments, R^{3B.1}, R^{3B.2}, and R^{3B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{3B.1}, R^{3B.2}, and R^{3B.3}, respectively.

In embodiments, when R^{3C} is substituted, R^{3C} is substituted with one or more first substituent groups denoted by R^{3C.1} as explained in the definitions section above in the description of “first substituent group(s)”. In embodiments, when an R^{3C.1} substituent group is substituted, the R^{3C.1} substituent group is substituted with one or more second substituent groups denoted by R^{3C.2} as explained in the definitions section above in the description of “first substituent group(s)”. In embodiments, when an R^{3C.2} substituent group is substituted, the R^{3C.2} substituent group is substituted with one or more third substituent groups denoted by R^{3C.3} as explained in the definitions section above in the description of “first substituent group(s)”. In the above embodiments, R^{3C}, R^{3C.1}, R^{3C.2}, and R^{3C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{3C}, R^{3C.1}, R^{3C.2}, and R^{3C.3}, respectively.

In embodiments, when R^{3D} is substituted, R^{3D} is substituted with one or more first substituent groups denoted by R^{3D.1} as explained in the definitions section above in the description of “first substituent group(s)”. In embodiments, when an R^{3D.1} substituent group is substituted, the R^{3D.1} substituent group is substituted with one or more second substituent groups denoted by R^{3D.2} as explained in the definitions section above in the description of “first substituent group(s)”. In embodiments, when an R^{3D.2} substituent group is substituted, the R^{3D.2} substituent group is substituted with one or more third substituent groups denoted by R^{3D.3} as explained in the definitions section above in the description of “first substituent group(s)”. In the above embodiments, R^{3D}, R^{3D.1}, R^{3D.2}, and R^{3D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{3D}, R^{3D.1}, R^{3D.2}, and R^{3D.3}, respectively.

In embodiments, when R⁵ is substituted, R⁵ is substituted with one or more first substituent groups denoted by R^{5.1} as explained in the definitions section above in the description of “first substituent group(s)”. In embodiments, when an R^{5.1} substituent group is substituted, the R^{5.1} substituent group is substituted with one or more second substituent groups denoted by R^{5.2} as explained in the definitions section above in the description of “first substituent group(s)”. In embodiments, when an R^{5.2} substituent group is substituted, the R^{5.2} substituent group is substituted with one or more third substituent groups denoted by R^{5.3} as explained in the definitions section above in the description of “first substituent group(s)”. In the above embodiments, R⁵, R^{5.1}, R^{5.2}, and R^{5.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R⁵, R^{5.1}, R^{5.2}, and R^{5.3}, respectively.

In embodiments, when R^{5A} is substituted, R^{5A} is substituted with one or more first substituent groups denoted by R^{5A.1} as explained in the definitions section above in the description of “first substituent group(s)”. In embodiments, when an R^{5A.1} substituent group is substituted, the R^{5A.1} substituent group is substituted with one or more second substituent groups denoted by R^{5A.2} as explained in the definitions section above in the description of “first substituent group(s)”. In embodiments, when an R^{5A.2} substituent group is substituted, the R^{5A.2} substituent group is substituted with one or more third substituent groups denoted by R^{5A.3} as explained in the definitions section above in the description of “first substituent group(s)”. In the above embodiments, R^{5A}, R^{5A.1}, R^{5A.2}, and R^{5A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{5A}, R^{5A.1}, R^{5A.2}, and R^{5A.3}, respectively.

In embodiments, when R^{5B} is substituted, R^{5B} is substituted with one or more first substituent groups denoted by R^{5B.1} as explained in the definitions section above in the description of “first substituent group(s)”. In embodiments, when an R^{5B.1} substituent group is substituted, the R^{5B.1} substituent group is substituted with one or more second substituent groups denoted by R^{5B.2} as explained in the definitions section above in the description of “first substituent group(s)”. In embodiments, when an R^{5B.2} substituent group is substituted, the R^{5B.2} substituent group is substituted with one or more third substituent groups denoted by R^{5B.3} as explained in the definitions section above in the description of “first substituent group(s)”. In the above embodiments, R^{5B}, R^{5B.1}, R^{5B.2}, and R^{5B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{5B}, R^{5B.1}, R^{5B.2}, and R^{5B.3}, respectively.

In embodiments, when R^{5A} and R^{5B} substituents bonded to the same nitrogen atom are optionally joined to form a moiety that is substituted (e.g., a substituted heterocycloalkyl or substituted heteroaryl), the moiety is substituted with one or more first substituent groups denoted by R^{5A.1} as explained in the definitions section above in the description of “first substituent group(s)”. In embodiments, when an R^{5A.1} substituent group is substituted, the R^{5A.1} substituent group is substituted with one or more second substituent groups denoted by R^{5A.2} as explained in the definitions section above in the description of “first substituent group(s)”. In embodiments, when an R^{5A.2} substituent group is substituted, the R^{5A.2} substituent group is substituted with one or more third substituent groups denoted by R^{5A.3} as explained in the definitions section above in the description of “first substituent group(s)”. In the above embodiments, R^{5A.1}, R^{5A.2}, and R^{5A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{5A.1}, R^{5A.2}, and R^{5A.3}, respectively.

In embodiments, when R^{5A} and R^{5B} substituents bonded to the same nitrogen atom are optionally joined to form a moiety that is substituted (e.g., a substituted heterocycloalkyl or substituted heteroaryl), the moiety is substituted with one or more first substituent groups denoted by R^{5B.1} as explained in the definitions section above in the description of “first substituent group(s)”. In embodiments, when an R^{5B.1} substituent group is substituted, the R^{5B.1} substituent group is substituted with one or more second substituent groups denoted by R^{5B.2} as explained in the definitions section above in the description of “first substituent group(s)”. In embodiments, when an R^{5B.2} substituent group is substituted, the R^{5B.2} substituent group is substituted with one or more third substituent groups denoted by R^{5B.3} as explained in the definitions section above in the description of “first substituent group(s)”. In the above embodiments, R^{5B.1}, R^{5B.2}, and R^{5B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{5B.1}, R^{5B.2}, and R^{5B.3}, respectively.

In embodiments, when R^{5C} is substituted, R^{5C} is substituted with one or more first substituent groups denoted by R^{5C.1} as explained in the definitions section above in the description of “first substituent group(s)”. In embodiments, when an R^{5C.1} substituent group is substituted, the R^{5C.1} substituent group is substituted with one or more second substituent groups denoted by R^{5C.2} as explained in the definitions section above in the description of “first substituent group(s)”. In embodiments, when an R^{5C.2} substituent group is substituted, the R^{5C.2} substituent group is substituted with one or more third substituent groups denoted by R^{5C.3} as explained in the definitions section above in the description of “first substituent group(s)”. In the above embodiments, R^{5C}, R^{5C.1}, R^{5C.2}, and R^{5C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{5C}, R^{5C.1}, R^{5C.2}, and R^{5C.3}, respectively.

In embodiments, when R^{5D} is substituted, R^{5D} is substituted with one or more first substituent groups denoted by R^{5D.1} as explained in the definitions section above in the description of “first substituent group(s)”. In embodiments, when an R^{5D.1} substituent group is substituted, the R^{5D.1} substituent group is substituted with one or more second substituent groups denoted by R^{5D.2} as explained in the definitions section above in the description of “first substituent group(s)”. In embodiments, when an R^{5D.2} substituent group is substituted, the R^{5D.2} substituent group is substituted with one or more third substituent groups denoted by R^{5D.3} as explained in the definitions section above in the description of “first substituent group(s)”. In the above embodiments, R^{5D}, R^{5D.1}, R^{5D.2}, and R^{5D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{5D}, R^{5D.1}, R^{5D.2}, and R^{5D.3}, respectively.

In embodiments, when R⁶ is substituted, R⁶ is substituted with one or more first substituent groups denoted by R^{6.1} as explained in the definitions section above in the description of “first substituent group(s)”. In embodiments, when an R^{6.1} substituent group is substituted, the R^{6.1} substituent group is substituted with one or more second substituent groups denoted by R^{6.2} as explained in the definitions section above in the description of “first substituent group(s)”. In embodiments, when an R^{6.2} substituent group is substituted, the R^{6.2} substituent group is substituted with one or more third substituent groups denoted by R^{6.3} as explained in the definitions section above in the description of “first substituent group(s)”. In the above embodiments, R⁶, R^{6.1}, R^{6.2}, and R^{6.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R⁶, R^{6.1}, R^{6.2}, and R^{6.3}, respectively.

In embodiments, when R^{6A} is substituted, R^{6A} is substituted with one or more first substituent groups denoted by R^{6A.1} as explained in the definitions section above in the description of “first substituent group(s)”. In embodiments, when an R^{6A.1} substituent group is substituted, the R^{6A.1} substituent group is substituted with one or more second substituent groups denoted by R^{6A.2} as explained in the definitions section above in the description of “first substituent group(s)”. In embodiments, when an R^{6A.2} substituent group is substituted, the R^{6A.2} substituent group is substituted with one or more third substituent groups denoted by R^{6A.3} as explained in the definitions section above in the description of “first substituent group(s)”. In the above embodiments, R^{6A}, R^{6A.1}, R^{6A.2}, and R^{6A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{6A}, R^{6A.1}, R^{6A.2}, and R^{6A.3}, respectively.

In embodiments, when R^{6B} is substituted, R^{B} is substituted with one or more first substituent groups denoted by R^{6B.1} as explained in the definitions section above in the description of “first substituent group(s)”. In embodiments, when an R^{6B.1} substituent group is substituted, the R^{6B.1} substituent group is substituted with one or more second substituent groups denoted by R^{6B.2} as explained in the definitions section above in the description of “first substituent group(s)”. In embodiments, when an R^{6B.2} substituent group is substituted, the R^{6B.2} substituent group is substituted with one or more third substituent groups denoted by R^{6B.3} as explained in the definitions section above in the description of “first substituent group(s)”. In the above embodiments, R^{6B}, R^{6B.1}, R^{6B.2}, and R^{6B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{6B}, R^{WW.1}, R^{6B.2}, and R^{6B.3}, respectively.

In embodiments, when R^{6A} and R^{6B} substituents bonded to the same nitrogen atom are optionally joined to form a moiety that is substituted (e.g., a substituted heterocycloalkyl or substituted heteroaryl), the moiety is substituted with one or more first substituent groups denoted by R^{6A.1} as explained in the definitions section above in the description of “first substituent group(s)”. In embodiments, when an R^{6A.1} substituent group is substituted, the R^{6A.1} substituent group is substituted with one or more second substituent groups denoted by R^{6A.2} as explained in the definitions section above in the description of “first substituent group(s)”. In embodiments, when an R^{6A.2} substituent group is substituted, the R^{6A.2} substituent group is substituted with one or more third substituent groups denoted by R^{6A.3} as explained in the definitions section above in the description of “first substituent group(s)”. In the above embodiments, R^{6A.1}, R^{6A.2}, and R^{6A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{6A.1}, R^{6A.2}, and R^{6A.3}, respectively.

In embodiments, when R^{6A} and R^{6B} substituents bonded to the same nitrogen atom are optionally joined to form a moiety that is substituted (e.g., a substituted heterocycloalkyl or substituted heteroaryl), the moiety is substituted with one or more first substituent groups denoted by R^{6B.1} as explained in the definitions section above in the description of “first substituent group(s)”. In embodiments, when an R^{6B.1} substituent group is substituted, the R^{6B.1} substituent group is substituted with one or more second substituent groups denoted by R^{6B.2} as explained in the definitions section above in the description of “first substituent group(s)”. In embodiments, when an R^{6B.2} substituent group is substituted, the R^{6B.2} substituent group is substituted with one or more third substituent groups denoted by R^{6B.3} as explained in the definitions section above in the description of “first substituent group(s)”. In the above embodiments, R^{6B.1}, R^{6B.2}, and R^{6B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{6B.1}, R^{6B.2}, and R^{6B.3}, respectively.

In embodiments, when R^{6C} is substituted, R^{6C} is substituted with one or more first substituent groups denoted by R^{6C.1} as explained in the definitions section above in the description of “first substituent group(s)”. In embodiments, when an R^{6C.1} substituent group is substituted, the R^{6C.1} substituent group is substituted with one or more second substituent groups denoted by R^{6C.2} as explained in the definitions section above in the description of “first substituent group(s)”. In embodiments, when an R^{6C.2} substituent group is substituted, the R^{6C.2} substituent group is substituted with one or more third substituent groups denoted by R^{6C.3} as explained in the definitions section above in the description of “first substituent group(s)”. In the above embodiments, R^{6C}, R^{6C.1}, R^{6C.2}, and R^{6C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{6C}, R^{6C.1}, R^{6C.2}, and R^{6C.3}, respectively.

In embodiments, when R^{6D} is substituted, R^{6D} is substituted with one or more first substituent groups denoted by R^{6D.1} as explained in the definitions section above in the description of “first substituent group(s)”. In embodiments, when an R^{6D.1} substituent group is substituted, the R^{6D.1} substituent group is substituted with one or more second substituent groups denoted by R^{6D.2} as explained in the definitions section above in the description of “first substituent group(s)”. In embodiments, when an R^{6D.2} substituent group is substituted, the R^{6D.2} substituent group is substituted with one or more third substituent groups denoted by R^{6D.3} as explained in the definitions section above in the description of “first substituent group(s)”. In the above embodiments, R^{6D}, R^{6D.1}, R^{6D.2}, and R^{6D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{6D}, R^{6D.1}, R^{6D.2}, and R^{6D.3}, respectively.

In embodiments, when L¹ is substituted, L¹ is substituted with one or more first substituent groups denoted by R^{L1.1} as explained in the definitions section above in the description of “first substituent group(s)”. In embodiments, when an R^{L1.1} substituent group is substituted, the R^{L1.1} substituent group is substituted with one or more second substituent groups denoted by R^{L1.2} as explained in the definitions section above in the description of “first substituent group(s)”. In embodiments, when an R^{L1.2} substituent group is substituted, the R^{L1.2} substituent group is substituted with one or more third substituent groups denoted by R^{L1.3} as explained in the definitions section above in the description of “first substituent group(s)”. In the above embodiments, L¹, R^{L1.1}, R^{L1.2}, and R^{L1.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L¹, R^{L1.1}, R^{L1.2}, and R^{L.3}, respectively.

In embodiments, when L^{2A} is substituted, L^{2A} is substituted with one or more first substituent groups denoted by R^{L2A.1} as explained in the definitions section above in the description of “first substituent group(s)”. In embodiments, when an R^{L2A.1} substituent group is substituted, the R^{L2A.1} substituent group is substituted with one or more second substituent groups denoted by R^{L2A.2} as explained in the definitions section above in the description of “first substituent group(s)”. In embodiments, when an R^{L2A.2} substituent group is substituted, the R^{L2A.2} substituent group is substituted with one or more third substituent groups denoted by R^{L2A.3} as explained in the definitions section above in the description of “first substituent group(s)”. In the above embodiments, L^{2A}, R^{L2A.1}, R^{L2A.2}, and R^{L2A.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{2A}, R^{L2A.1}, R^{L2A.2}, and R^{L2A.3}, respectively.

In embodiments, when L^{2B} is substituted, L^{2B} is substituted with one or more first substituent groups denoted by R^{L2B.1} as explained in the definitions section above in the description of “first substituent group(s)”. In embodiments, when an R^{L2B.1} substituent group is substituted, the R^{L2B.1} substituent group is substituted with one or more second substituent groups denoted by R^{L2B.2} as explained in the definitions section above in the description of “first substituent group(s)”. In embodiments, when an R^{L2B.2} substituent group is substituted, the R^{L2B.2} substituent group is substituted with one or more third substituent groups denoted by R^{L2B.3} as explained in the definitions section above in the description of “first substituent group(s)”. In the above embodiments, L^{2B}, R^{L2B.1}, R^{L2B.2}, and R^{L2B.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{2B}, R^{L2B.1}, R^{L2B.2}, and R^{L2B.3}, respectively.

In embodiments, when L^{2C} is substituted, L^{2C} is substituted with one or more first substituent groups denoted by R^{L2C.1} as explained in the definitions section above in the description of “first substituent group(s)”. In embodiments, when an R^{L2C.1} substituent group is substituted, the R^{L2C.1} substituent group is substituted with one or more second substituent groups denoted by R^{L2C.2} as explained in the definitions section above in the description of “first substituent group(s)”. In embodiments, when an R^{L2C.2} substituent group is substituted, the R^{L2C.2} substituent group is substituted with one or more third substituent groups denoted by R^{L2C.3} as explained in the definitions section above in the description of “first substituent group(s)”. In the above embodiments, L^{2C}, R^{L2C.1}, R^{L2C.2}, and R^{L2C.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{2C}, R^{L2C.1}, R^{L2C.2}, and R^{L2C.3}, respectively.

In embodiments, the compound is

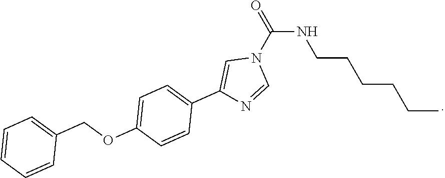 [SMILES: CCCCCCNC(=O)n1cnc(-c2ccc(OCc3ccccc3)cc2)c1]

In embodiments, the compound is

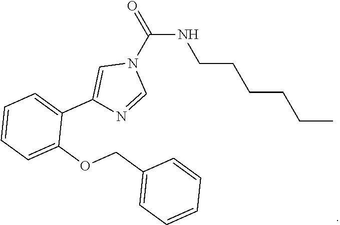 [SMILES: CCCCCCNC(=O)n1cnc(-c2ccccc2OCc2ccccc2)c1]

In embodiments, the compound is

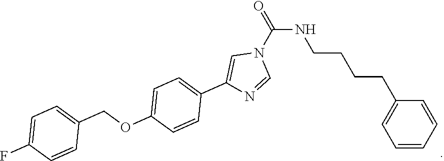 [SMILES: O=C(NCCCCc1ccccc1)n1cnc(-c2ccc(OCc3ccc(F)cc3)cc2)c1]

In embodiments, the compound is

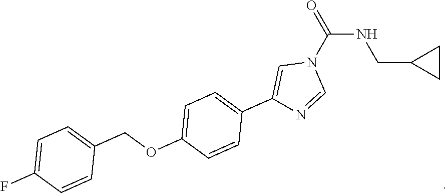 [SMILES: O=C(NCC1CC1)n1cnc(-c2ccc(OCc3ccc(F)cc3)cc2)c1]

In embodiments, the compound is

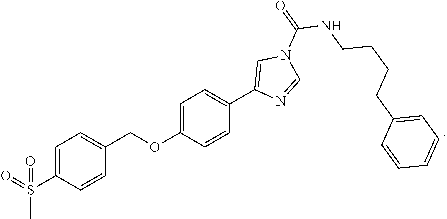 [SMILES: CS(=O)(=O)c1ccc(COc2ccc(-c3cn(C(=O)NCCCCc4ccccc4)cn3)cc2)cc1]

In embodiments, the compound is

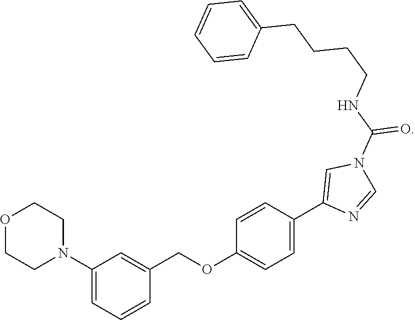 [SMILES: O=C(NCCCCc1ccccc1)n1cnc(-c2ccc(OCc3cccc(N4CCOCC4)c3)cc2)c1]

In embodiments, the compound is

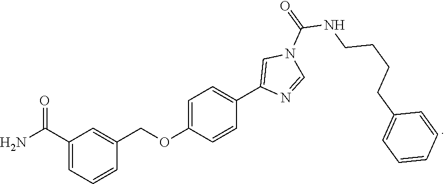 [SMILES: NC(=O)c1cccc(COc2ccc(-c3cn(C(=O)NCCCCc4ccccc4)cn3)cc2)c1]

In embodiments, the compound is

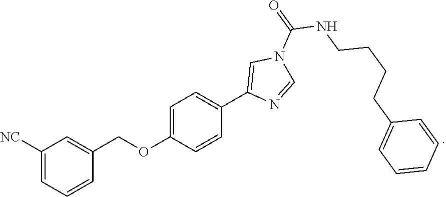 [SMILES: N#Cc1cccc(COc2ccc(-c3cn(C(=O)NCCCCc4ccccc4)cn3)cc2)c1]

In embodiments, the compound is

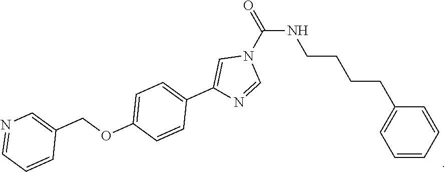 [SMILES: O=C(NCCCCc1ccccc1)n1cnc(-c2ccc(OCc3cccnc3)cc2)c1]

In embodiments, the compound is

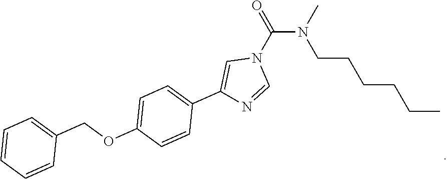 [SMILES: CCCCCCN(C)C(=O)n1cnc(-c2ccc(OCc3ccccc3)cc2)c1]

In embodiments, the compound is

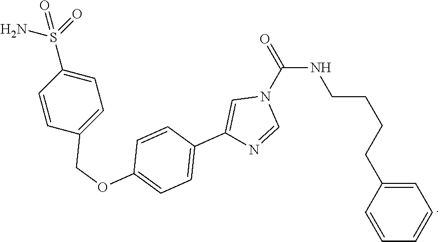 [SMILES: NS(=O)(=O)c1ccc(COc2ccc(-c3cn(C(=O)NCCCCc4ccccc4)cn3)cc2)cc1]

In embodiments, the compound is

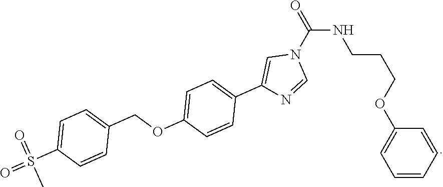 [SMILES: CS(=O)(=O)c1ccc(COc2ccc(-c3cn(C(=O)NCCCOc4ccccc4)cn3)cc2)cc1]

In embodiments, the compound is

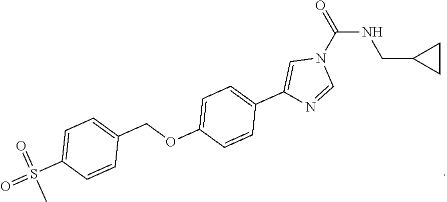 [SMILES: CS(=O)(=O)c1ccc(COc2ccc(-c3cn(C(=O)NCC4CC4)cn3)cc2)cc1]

In embodiments, the compound is

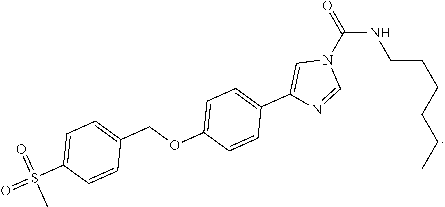 [SMILES: CCCCCCNC(=O)n1cnc(-c2ccc(OCc3ccc(S(C)(=O)=O)cc3)cc2)c1]

In embodiments, the compound is

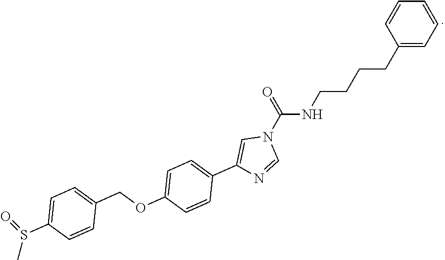 [SMILES: CS(=O)c1ccc(COc2ccc(-c3cn(C(=O)NCCCCc4ccccc4)cn3)cc2)cc1]

In embodiments, the compound is

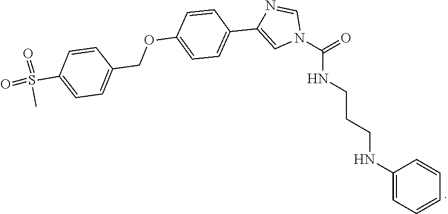 [SMILES: CS(=O)(=O)c1ccc(COc2ccc(-c3cn(C(=O)NCCCNc4ccccc4)cn3)cc2)cc1]

In embodiments, the compound is

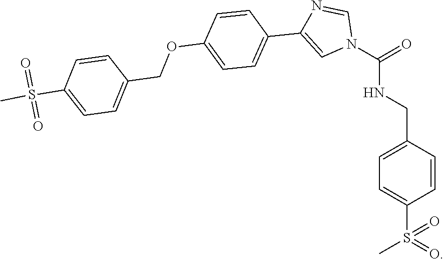 [SMILES: CS(=O)(=O)c1ccc(CNC(=O)n2cnc(-c3ccc(OCc4ccc(S(C)(=O)=O)cc4)cc3)c2)cc1]

In embodiments, the compound is

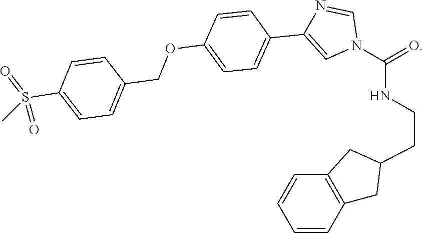 [SMILES: CS(=O)(=O)c1ccc(COc2ccc(-c3cn(C(=O)NCCC4Cc5ccccc5C4)cn3)cc2)cc1]

In embodiments, the compound is

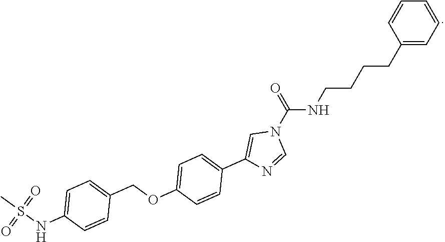 [SMILES: CS(=O)(=O)Nc1ccc(COc2ccc(-c3cn(C(=O)NCCCCc4ccccc4)cn3)cc2)cc1]

In embodiments, the compound is

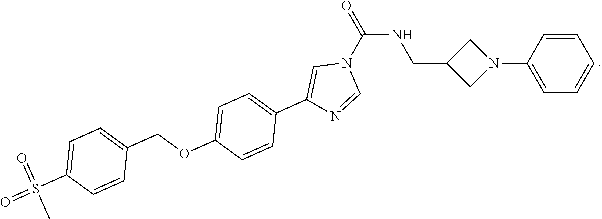 [SMILES: CS(=O)(=O)c1ccc(COc2ccc(-c3cn(C(=O)NCC4CN(c5ccccc5)C4)cn3)cc2)cc1]

In embodiments, the compound is

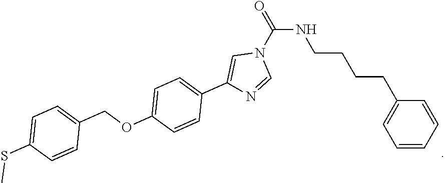 [SMILES: CSc1ccc(COc2ccc(-c3cn(C(=O)NCCCCc4ccccc4)cn3)cc2)cc1]

In embodiments, the compound is

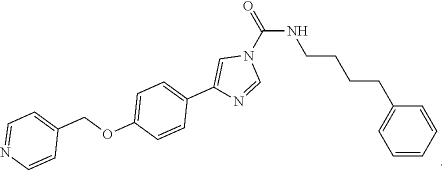 [SMILES: O=C(NCCCCc1ccccc1)n1cnc(-c2ccc(OCc3ccncc3)cc2)c1]

In embodiments, the compound is

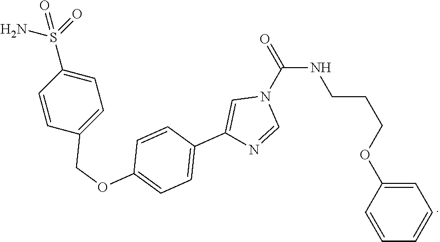 [SMILES: NS(=O)(=O)c1ccc(COc2ccc(-c3cn(C(=O)NCCCOc4ccccc4)cn3)cc2)cc1]

In embodiments, the compound is

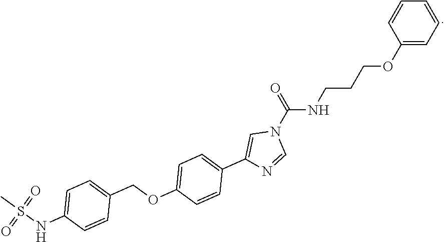 [SMILES: CS(=O)(=O)Nc1ccc(COc2ccc(-c3cn(C(=O)NCCCOc4ccccc4)cn3)cc2)cc1]

In embodiments, the compound is

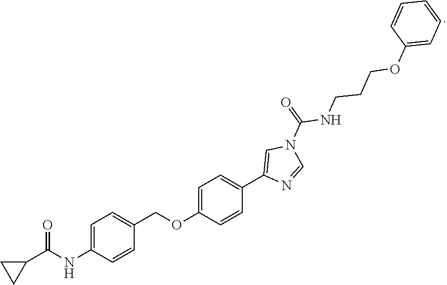 [SMILES: O=C(Nc1ccc(COc2ccc(-c3cn(C(=O)NCCCOc4ccccc4)cn3)cc2)cc1)C1CC1]

In embodiments, the compound is

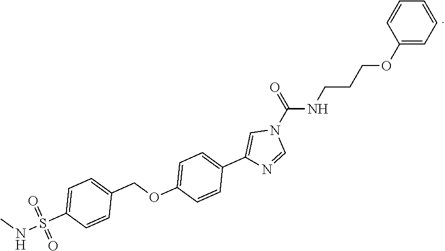 [SMILES: CNS(=O)(=O)c1ccc(COc2ccc(-c3cn(C(=O)NCCCOc4ccccc4)cn3)cc2)cc1]

In embodiments, the compound is

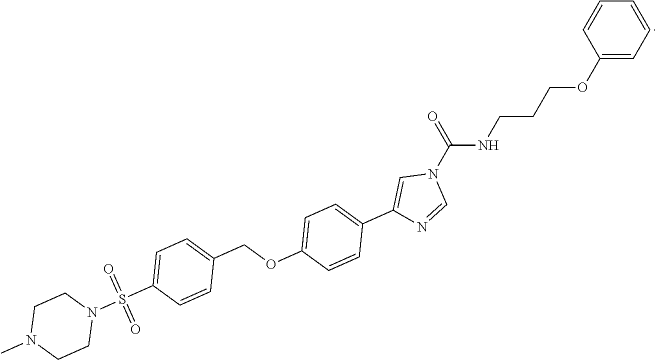 [SMILES: CN1CCN(S(=O)(=O)c2ccc(COc3ccc(-c4cn(C(=O)NCCCOc5ccccc5)cn4)cc3)cc2)CC1]

In embodiments, the compound is

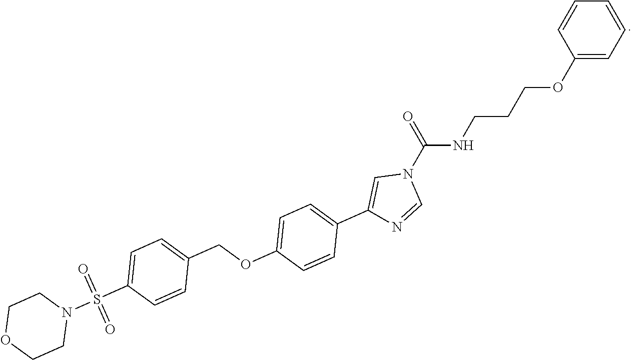 [SMILES: O=C(NCCCOc1ccccc1)n1cnc(-c2ccc(OCc3ccc(S(=O)(=O)N4CCOCC4)cc3)cc2)c1]

In embodiments, the compound is

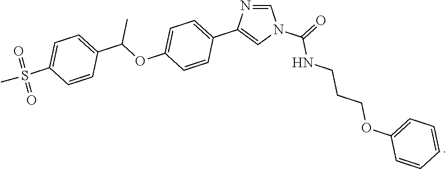 [SMILES: CC(Oc1ccc(-c2cn(C(=O)NCCCOc3ccccc3)cn2)cc1)c1ccc(S(C)(=O)=O)cc1]

In embodiments, the compound is

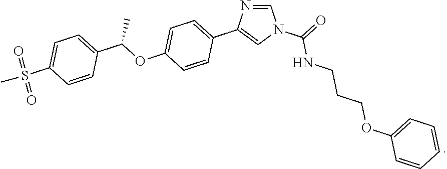 [SMILES: C[C@H](Oc1ccc(-c2cn(C(=O)NCCCOc3ccccc3)cn2)cc1)c1ccc(S(C)(=O)=O)cc1]

In embodiments, the compound is

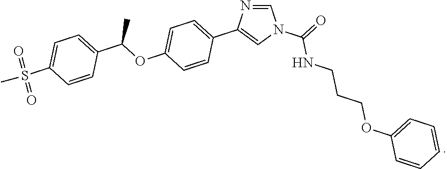 [SMILES: C[C@@H](Oc1ccc(-c2cn(C(=O)NCCCOc3ccccc3)cn2)cc1)c1ccc(S(C)(=O)=O)cc1]

In embodiments, the compound is

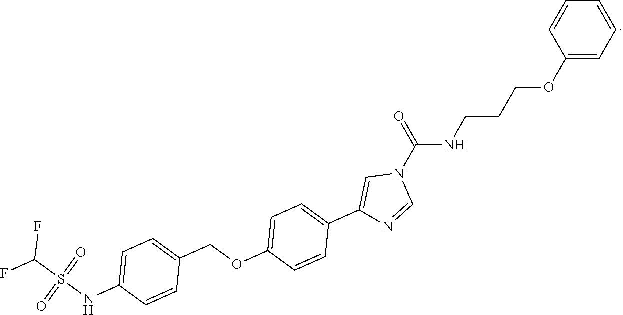 [SMILES: O=C(NCCCOc1ccccc1)n1cnc(-c2ccc(OCc3ccc(NS(=O)(=O)C(F)F)cc3)cc2)c1]

In embodiments, the compound is

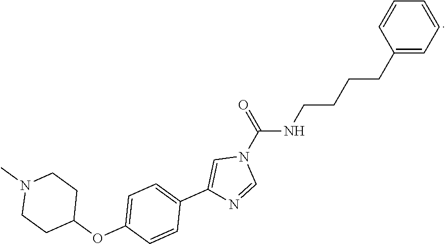 [SMILES: CN1CCC(Oc2ccc(-c3cn(C(=O)NCCCCc4ccccc4)cn3)cc2)CC1]

In embodiments, the compound is

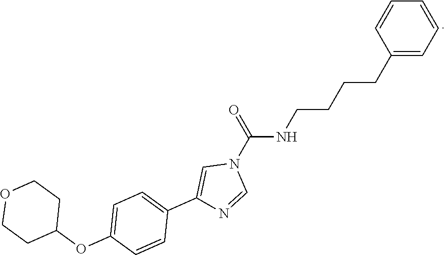 [SMILES: O=C(NCCCCc1ccccc1)n1cnc(-c2ccc(OC3CCOCC3)cc2)c1]

In embodiments, the compound is

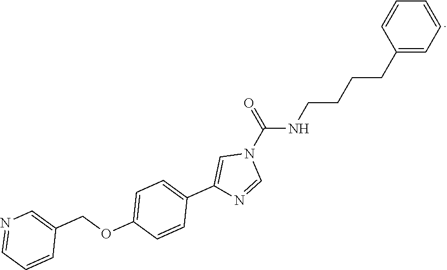 [SMILES: O=C(NCCCCc1ccccc1)n1cnc(-c2ccc(OCc3cccnc3)cc2)c1]

In embodiments, the compound is

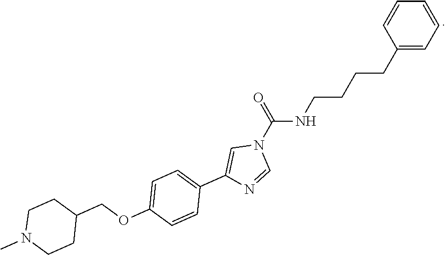 [SMILES: CN1CCC(COc2ccc(-c3cn(C(=O)NCCCCc4ccccc4)cn3)cc2)CC1]

In embodiments, the compound is

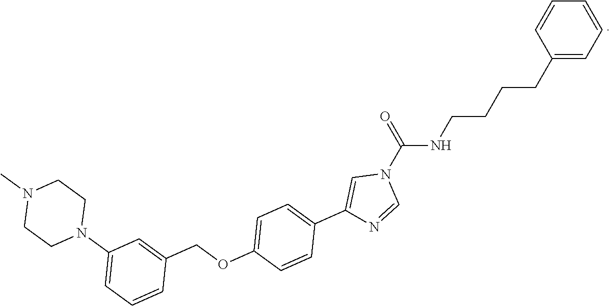 [SMILES: CN1CCN(c2cccc(COc3ccc(-c4cn(C(=O)NCCCCc5ccccc5)cn4)cc3)c2)CC1]

In embodiments, the compound is

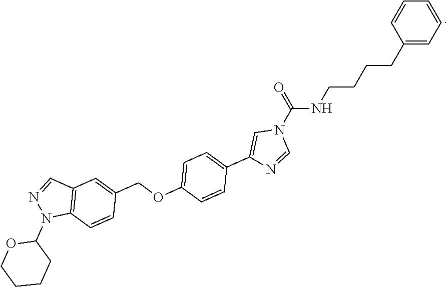 [SMILES: O=C(NCCCCc1ccccc1)n1cnc(-c2ccc(OCc3ccc4c(cnn4C4CCCCO4)c3)cc2)c1]

In embodiments, the compound is

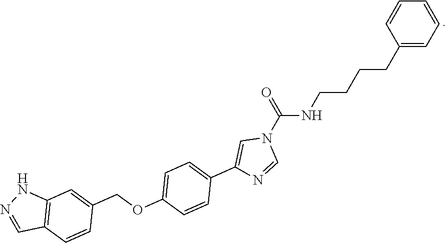 [SMILES: O=C(NCCCCc1ccccc1)n1cnc(-c2ccc(OCc3ccc4cn[nH]c4c3)cc2)c1]

In embodiments, the compound is

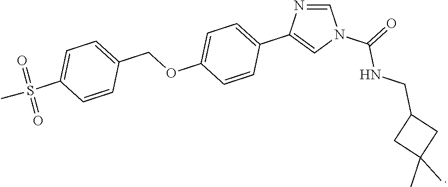

In embodiments, the compound is

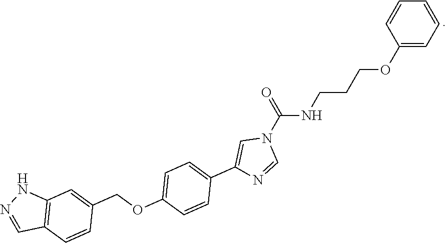 [SMILES: O=C(NCCCOc1ccccc1)n1cnc(-c2ccc(OCc3ccc4cn[nH]c4c3)cc2)c1]

In embodiments, the compound is

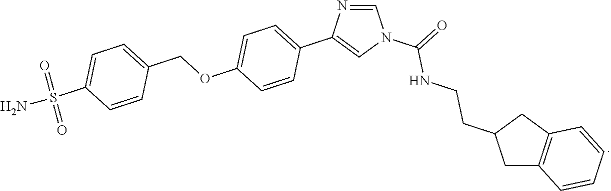 [SMILES: NS(=O)(=O)c1ccc(COc2ccc(-c3cn(C(=O)NCCC4Cc5ccccc5C4)cn3)cc2)cc1]

In embodiments, the compound is

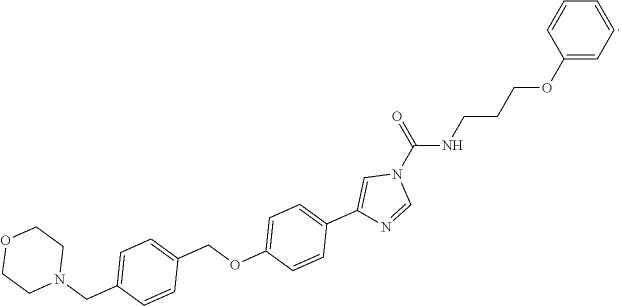 [SMILES: O=C(NCCCOc1ccccc1)n1cnc(-c2ccc(OCc3ccc(CN4CCOCC4)cc3)cc2)c1]

In embodiments, the compound is

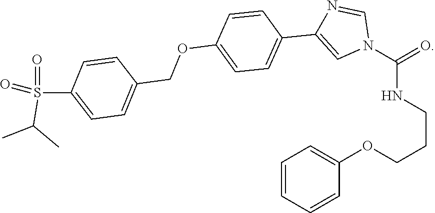 [SMILES: CC(C)S(=O)(=O)c1ccc(COc2ccc(-c3cn(C(=O)NCCCOc4ccccc4)cn3)cc2)cc1]

In embodiments, the compound is

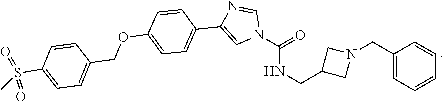 [SMILES: CS(=O)(=O)c1ccc(COc2ccc(-c3cn(C(=O)NCC4CN(Cc5ccccc5)C4)cn3)cc2)cc1]

In embodiments, the compound is

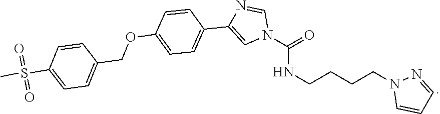 [SMILES: CS(=O)(=O)c1ccc(COc2ccc(-c3cn(C(=O)NCCCCn4cccn4)cn3)cc2)cc1]

In embodiments, the compound is

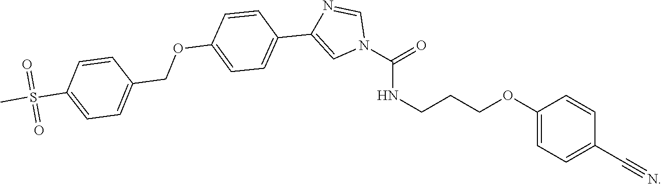 [SMILES: CS(=O)(=O)c1ccc(COc2ccc(-c3cn(C(=O)NCCCOc4ccc(C#N)cc4)cn3)cc2)cc1]

In embodiments, the compound is

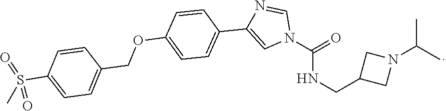 [SMILES: CC(C)N1CC(CNC(=O)n2cnc(-c3ccc(OCc4ccc(S(C)(=O)=O)cc4)cc3)c2)C1]

In embodiments, the compound is

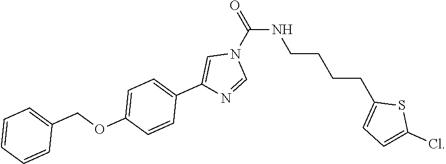 [SMILES: O=C(NCCCCc1ccc(Cl)s1)n1cnc(-c2ccc(OCc3ccccc3)cc2)c1]

In embodiments, the compound is

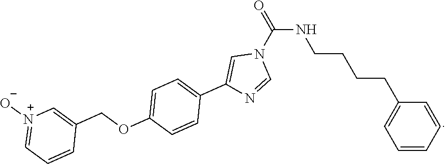 [SMILES: O=C(NCCCCc1ccccc1)n1cnc(-c2ccc(OCc3ccc[n+]([O-])c3)cc2)c1]

In embodiments, the compound is

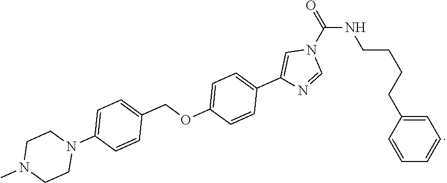 [SMILES: CN1CCN(c2ccc(COc3ccc(-c4cn(C(=O)NCCCCc5ccccc5)cn4)cc3)cc2)CC1]

In embodiments, the compound is

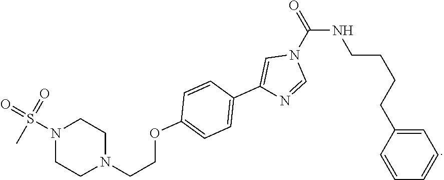 [SMILES: CS(=O)(=O)N1CCN(CCOc2ccc(-c3cn(C(=O)NCCCCc4ccccc4)cn3)cc2)CC1]

In embodiments, the compound is

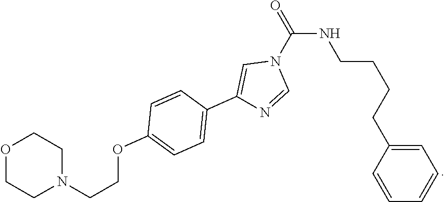 [SMILES: O=C(NCCCCc1ccccc1)n1cnc(-c2ccc(OCCN3CCOCC3)cc2)c1]

In embodiments, the compound is

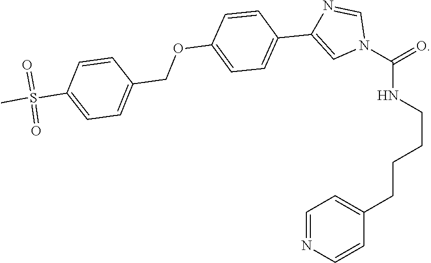 [SMILES: CS(=O)(=O)c1ccc(COc2ccc(-c3cn(C(=O)NCCCCc4ccncc4)cn3)cc2)cc1]

In embodiments, the compound is

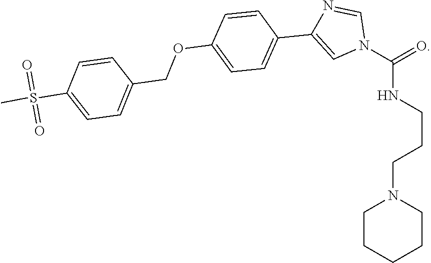 [SMILES: CS(=O)(=O)c1ccc(COc2ccc(-c3cn(C(=O)NCCCN4CCCCC4)cn3)cc2)cc1]

In embodiments, the compound is

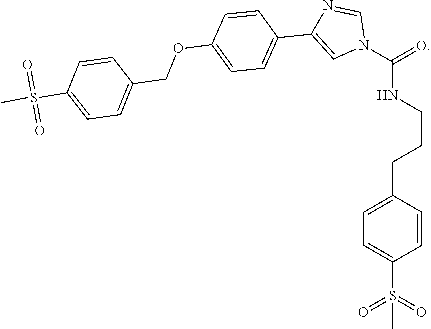 [SMILES: CS(=O)(=O)c1ccc(CCCNC(=O)n2cnc(-c3ccc(OCc4ccc(S(C)(=O)=O)cc4)cc3)c2)cc1]

In embodiments, the compound is

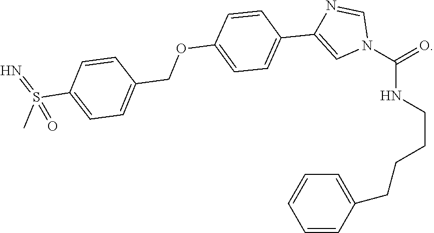 [SMILES: CS(=N)(=O)c1ccc(COc2ccc(-c3cn(C(=O)NCCCCc4ccccc4)cn3)cc2)cc1]

In embodiments, the compound is

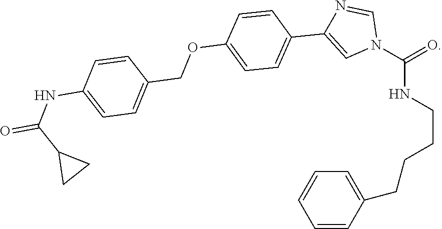 [SMILES: O=C(Nc1ccc(COc2ccc(-c3cn(C(=O)NCCCCc4ccccc4)cn3)cc2)cc1)C1CC1]

In embodiments, the compound is

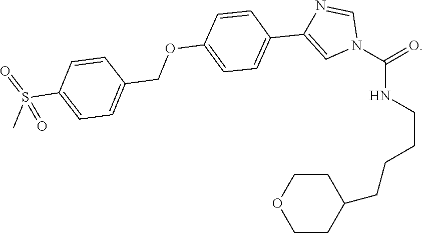 [SMILES: CS(=O)(=O)c1ccc(COc2ccc(-c3cn(C(=O)NCCCCC4CCOCC4)cn3)cc2)cc1]

In embodiments, the compound is

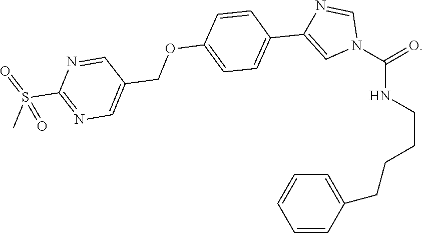 [SMILES: CS(=O)(=O)c1ncc(COc2ccc(-c3cn(C(=O)NCCCCc4ccccc4)cn3)cc2)cn1]

In embodiments, the compound is

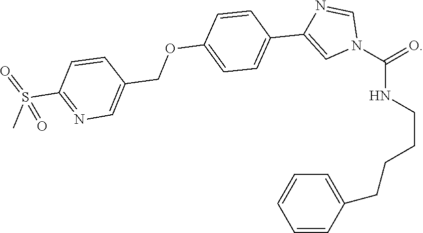 [SMILES: CS(=O)(=O)c1ccc(COc2ccc(-c3cn(C(=O)NCCCCc4ccccc4)cn3)cc2)cn1]

In embodiments, the compound is

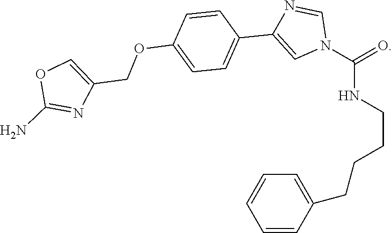 [SMILES: Nc1nc(COc2ccc(-c3cn(C(=O)NCCCCc4ccccc4)cn3)cc2)co1]

In embodiments, the compound is

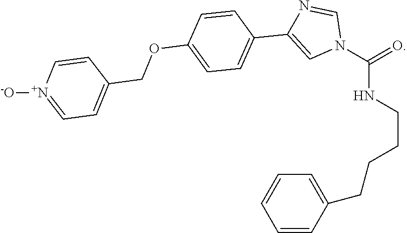

In embodiments, the compound is

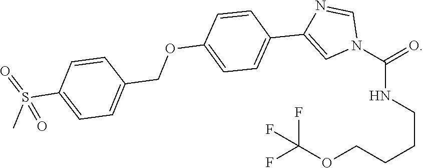 [SMILES: CS(=O)(=O)c1ccc(COc2ccc(-c3cn(C(=O)NCCCCOC(F)(F)F)cn3)cc2)cc1]

In embodiments, the compound is

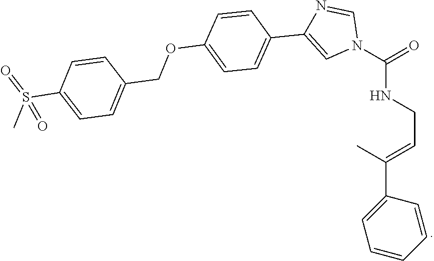 [SMILES: C/C(=C\CNC(=O)n1cnc(-c2ccc(OCc3ccc(S(C)(=O)=O)cc3)cc2)c1)c1ccccc1]

In embodiments, the compound is

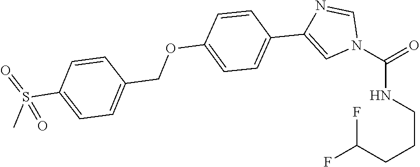 [SMILES: CS(=O)(=O)c1ccc(COc2ccc(-c3cn(C(=O)NCCCC(F)F)cn3)cc2)cc1]

In embodiments, the compound is

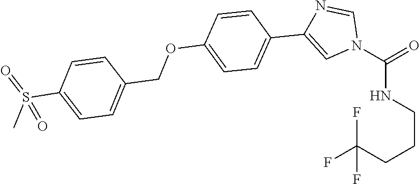 [SMILES: CS(=O)(=O)c1ccc(COc2ccc(-c3cn(C(=O)NCCCC(F)(F)F)cn3)cc2)cc1]

In embodiments, the compound is

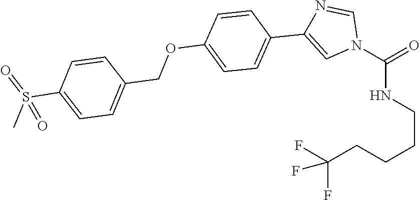 [SMILES: CS(=O)(=O)c1ccc(COc2ccc(-c3cn(C(=O)NCCCCC(F)(F)F)cn3)cc2)cc1]

In embodiments, the compound is

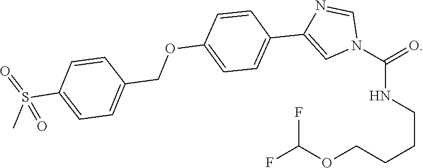 [SMILES: CS(=O)(=O)c1ccc(COc2ccc(-c3cn(C(=O)NCCCCOC(F)F)cn3)cc2)cc1]

In embodiments, the compound is

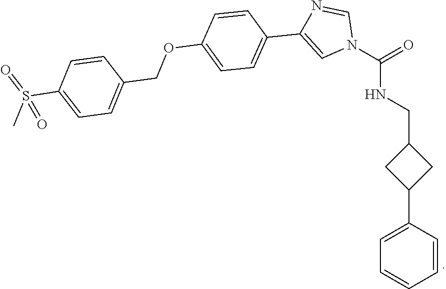 [SMILES: CS(=O)(=O)c1ccc(COc2ccc(-c3cn(C(=O)NCC4CC(c5ccccc5)C4)cn3)cc2)cc1]

In embodiments, the compound is

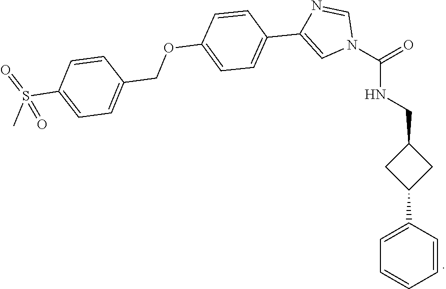 [SMILES: CS(=O)(=O)c1ccc(COc2ccc(-c3cn(C(=O)NC[C@H]4C[C@H](c5ccccc5)C4)cn3)cc2)cc1]

In embodiments, the compound is

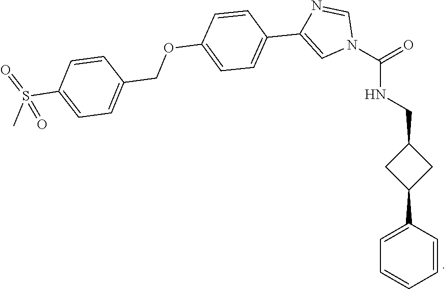 [SMILES: CS(=O)(=O)c1ccc(COc2ccc(-c3cn(C(=O)NC[C@H]4C[C@@H](c5ccccc5)C4)cn3)cc2)cc1]

In embodiments, the compound is

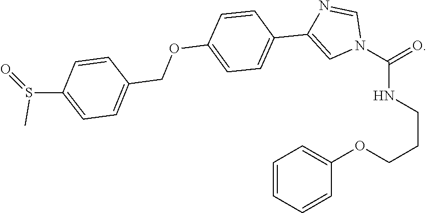 [SMILES: CS(=O)c1ccc(COc2ccc(-c3cn(C(=O)NCCCOc4ccccc4)cn3)cc2)cc1]

In embodiments, the compound is

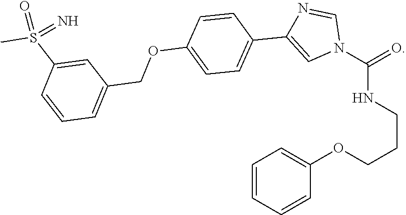 [SMILES: CS(=N)(=O)c1cccc(COc2ccc(-c3cn(C(=O)NCCCOc4ccccc4)cn3)cc2)c1]

In embodiments, the compound is

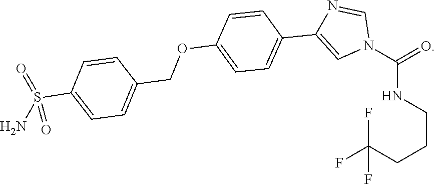 [SMILES: NS(=O)(=O)c1ccc(COc2ccc(-c3cn(C(=O)NCCCC(F)(F)F)cn3)cc2)cc1]

In embodiments, the compound is

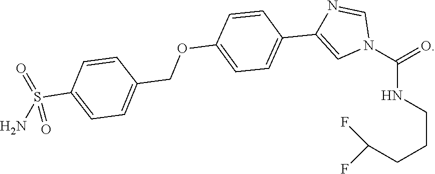 [SMILES: NS(=O)(=O)c1ccc(COc2ccc(-c3cn(C(=O)NCCCC(F)F)cn3)cc2)cc1]

In embodiments, the compound is

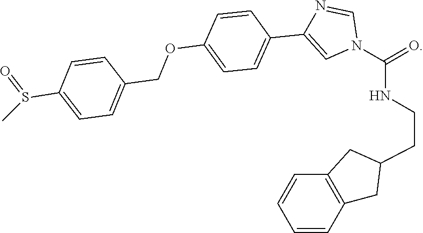 [SMILES: CS(=O)c1ccc(COc2ccc(-c3cn(C(=O)NCCC4Cc5ccccc5C4)cn3)cc2)cc1]

In embodiments, the compound is

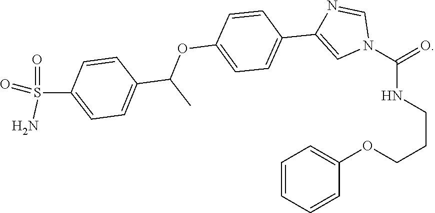 [SMILES: CC(Oc1ccc(-c2cn(C(=O)NCCCOc3ccccc3)cn2)cc1)c1ccc(S(N)(=O)=O)cc1]

In embodiments, the compound is

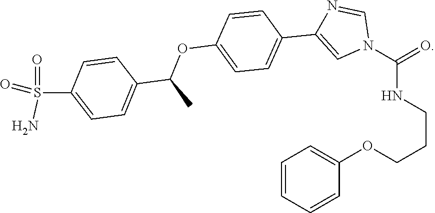 [SMILES: C[C@H](Oc1ccc(-c2cn(C(=O)NCCCOc3ccccc3)cn2)cc1)c1ccc(S(N)(=O)=O)cc1]

In embodiments, the compound is

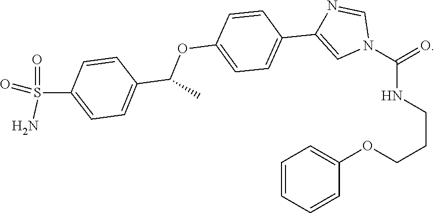 [SMILES: C[C@@H](Oc1ccc(-c2cn(C(=O)NCCCOc3ccccc3)cn2)cc1)c1ccc(S(N)(=O)=O)cc1]

In embodiments, the compound is

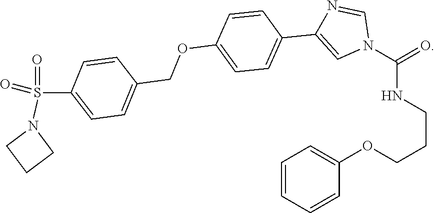 [SMILES: O=C(NCCCOc1ccccc1)n1cnc(-c2ccc(OCc3ccc(S(=O)(=O)N4CCC4)cc3)cc2)c1]

In embodiments, the compound is

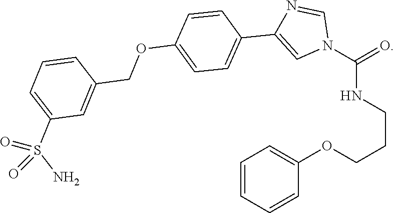 [SMILES: NS(=O)(=O)c1cccc(COc2ccc(-c3cn(C(=O)NCCCOc4ccccc4)cn3)cc2)c1]

In embodiments, the compound is

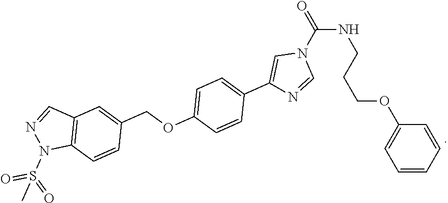 [SMILES: CS(=O)(=O)n1ncc2cc(COc3ccc(-c4cn(C(=O)NCCCOc5ccccc5)cn4)cc3)ccc21]

In embodiments, the compound is

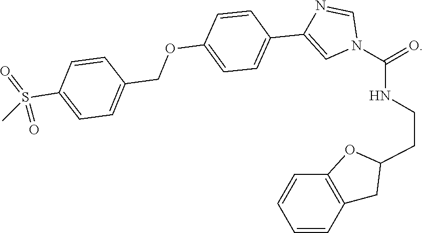 [SMILES: CS(=O)(=O)c1ccc(COc2ccc(-c3cn(C(=O)NCCC4Cc5ccccc5O4)cn3)cc2)cc1]

In embodiments, the compound is

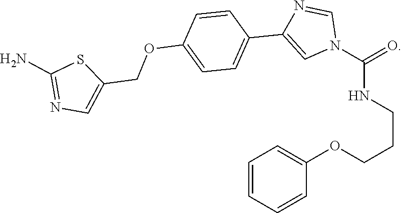 [SMILES: Nc1ncc(COc2ccc(-c3cn(C(=O)NCCCOc4ccccc4)cn3)cc2)s1]

In embodiments, the compound is

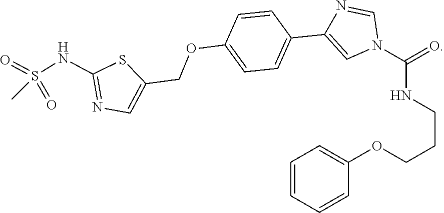 [SMILES: CS(=O)(=O)Nc1ncc(COc2ccc(-c3cn(C(=O)NCCCOc4ccccc4)cn3)cc2)s1]

In embodiments, the compound is

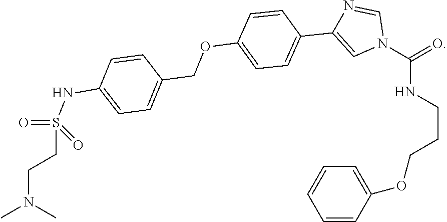 [SMILES: CN(C)CCS(=O)(=O)Nc1ccc(COc2ccc(-c3cn(C(=O)NCCCOc4ccccc4)cn3)cc2)cc1]

In embodiments, the compound is

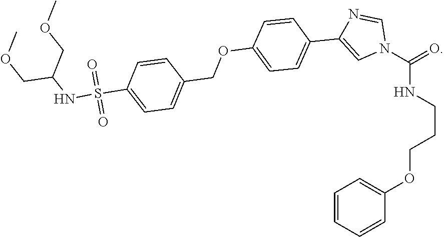 [SMILES: COCC(COC)NS(=O)(=O)c1ccc(COc2ccc(-c3cn(C(=O)NCCCOc4ccccc4)cn3)cc2)cc1]

In embodiments, the compound is

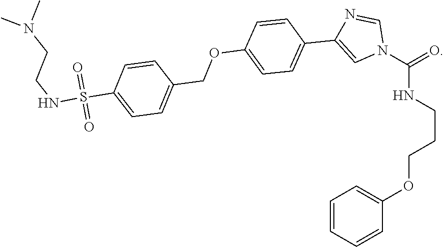 [SMILES: CN(C)CCNS(=O)(=O)c1ccc(COc2ccc(-c3cn(C(=O)NCCCOc4ccccc4)cn3)cc2)cc1]

In embodiments, the compound is

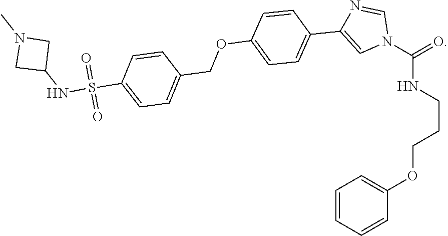 [SMILES: CN1CC(NS(=O)(=O)c2ccc(COc3ccc(-c4cn(C(=O)NCCCOc5ccccc5)cn4)cc3)cc2)C1]

In embodiments, the compound is

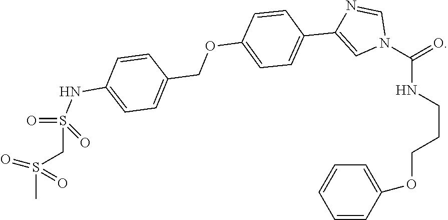 [SMILES: CS(=O)(=O)CS(=O)(=O)Nc1ccc(COc2ccc(-c3cn(C(=O)NCCCOc4ccccc4)cn3)cc2)cc1]

In embodiments, the compound is

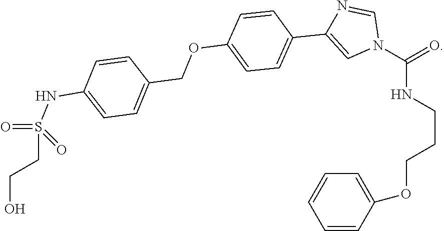 [SMILES: O=C(NCCCOc1ccccc1)n1cnc(-c2ccc(OCc3ccc(NS(=O)(=O)CCO)cc3)cc2)c1]

In embodiments, the compound is

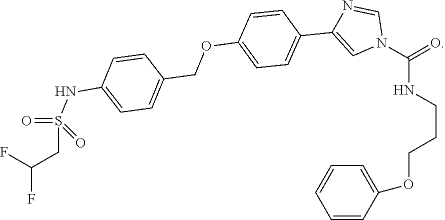 [SMILES: O=C(NCCCOc1ccccc1)n1cnc(-c2ccc(OCc3ccc(NS(=O)(=O)CC(F)F)cc3)cc2)c1]

In embodiments, the compound is

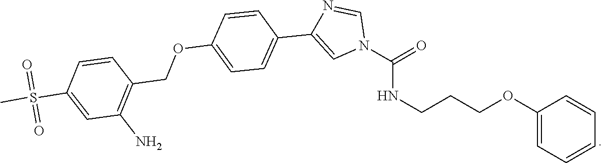 [SMILES: CS(=O)(=O)c1ccc(COc2ccc(-c3cn(C(=O)NCCCOc4ccccc4)cn3)cc2)c(N)c1]

In embodiments, the compound is

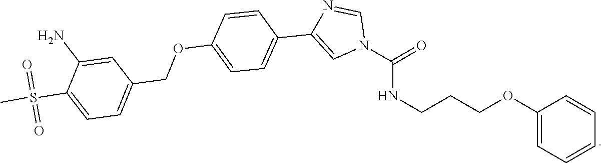 [SMILES: CS(=O)(=O)c1ccc(COc2ccc(-c3cn(C(=O)NCCCOc4ccccc4)cn3)cc2)cc1N]

In embodiments, the compound is

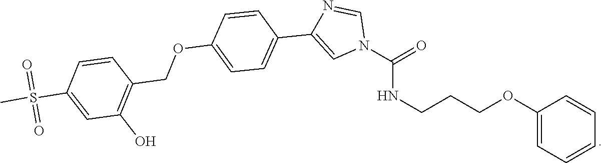 [SMILES: CS(=O)(=O)c1ccc(COc2ccc(-c3cn(C(=O)NCCCOc4ccccc4)cn3)cc2)c(O)c1]

In embodiments, the compound is

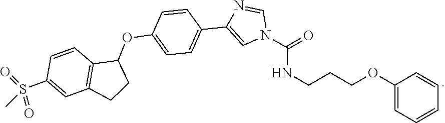 [SMILES: CS(=O)(=O)c1ccc2c(c1)CCC2Oc1ccc(-c2cn(C(=O)NCCCOc3ccccc3)cn2)cc1]

In embodiments, the compound is

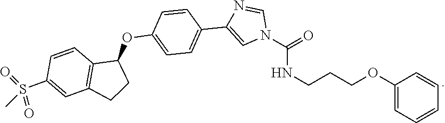 [SMILES: CS(=O)(=O)c1ccc2c(c1)CC[C@@H]2Oc1ccc(-c2cn(C(=O)NCCCOc3ccccc3)cn2)cc1]

In embodiments, the compound is

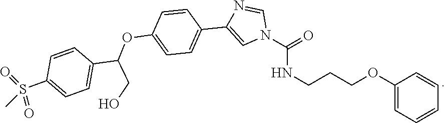 [SMILES: CS(=O)(=O)c1ccc(C(CO)Oc2ccc(-c3cn(C(=O)NCCCOc4ccccc4)cn3)cc2)cc1]

In embodiments, the compound is

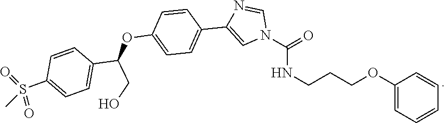 [SMILES: CS(=O)(=O)c1ccc([C@H](CO)Oc2ccc(-c3cn(C(=O)NCCCOc4ccccc4)cn3)cc2)cc1]

In embodiments, the compound is

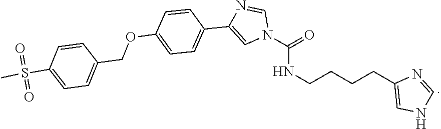 [SMILES: CS(=O)(=O)c1ccc(COc2ccc(-c3cn(C(=O)NCCCCc4c[nH]cn4)cn3)cc2)cc1]

In embodiments, the compound is

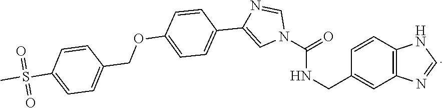 [SMILES: CS(=O)(=O)c1ccc(COc2ccc(-c3cn(C(=O)NCc4ccc5[nH]cnc5c4)cn3)cc2)cc1]

In embodiments, the compound is

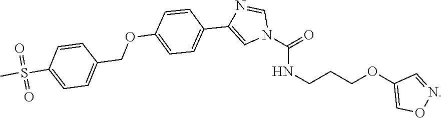 [SMILES: CS(=O)(=O)c1ccc(COc2ccc(-c3cn(C(=O)NCCCOc4cnoc4)cn3)cc2)cc1]

In embodiments, the compound is

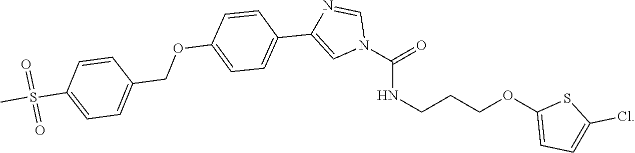 [SMILES: CS(=O)(=O)c1ccc(COc2ccc(-c3cn(C(=O)NCCCOc4ccc(Cl)s4)cn3)cc2)cc1]

In embodiments, the compound is

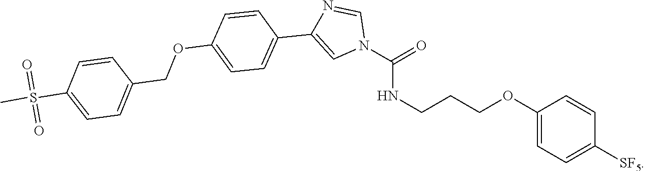

In embodiments, the compound is

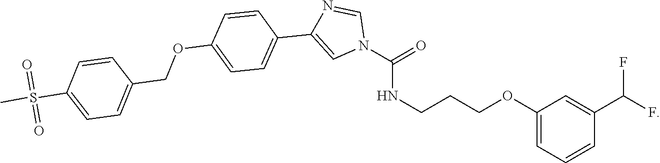 [SMILES: CS(=O)(=O)c1ccc(COc2ccc(-c3cn(C(=O)NCCCOc4cccc(C(F)F)c4)cn3)cc2)cc1]

In embodiments, the compound is

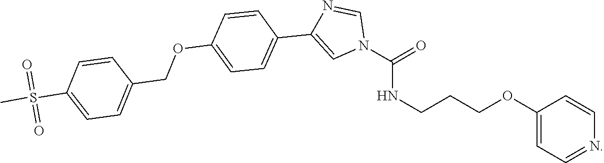 [SMILES: CS(=O)(=O)c1ccc(COc2ccc(-c3cn(C(=O)NCCCOc4ccncc4)cn3)cc2)cc1]

In embodiments, the compound is

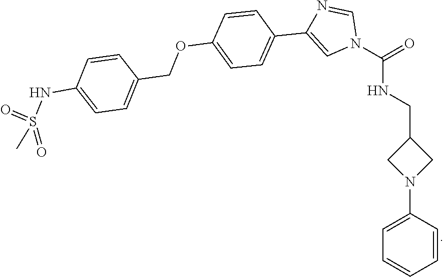 [SMILES: CS(=O)(=O)Nc1ccc(COc2ccc(-c3cn(C(=O)NCC4CN(c5ccccc5)C4)cn3)cc2)cc1]

In embodiments, the compound is

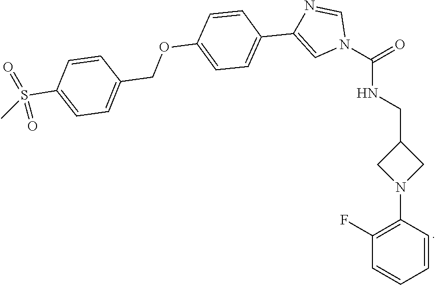 [SMILES: CS(=O)(=O)c1ccc(COc2ccc(-c3cn(C(=O)NCC4CN(c5ccccc5F)C4)cn3)cc2)cc1]

In embodiments, the compound is

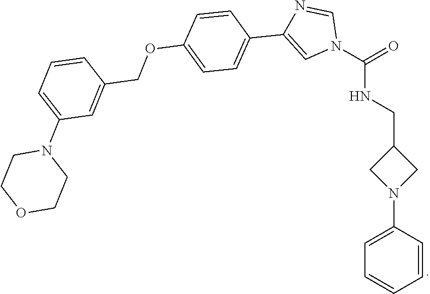 [SMILES: O=C(NCC1CN(c2ccccc2)C1)n1cnc(-c2ccc(OCc3cccc(N4CCOCC4)c3)cc2)c1]

In embodiments, the compound is

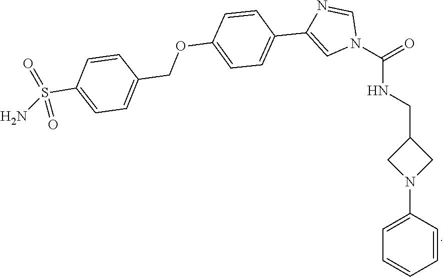 [SMILES: NS(=O)(=O)c1ccc(COc2ccc(-c3cn(C(=O)NCC4CN(c5ccccc5)C4)cn3)cc2)cc1]

In embodiments, the compound is

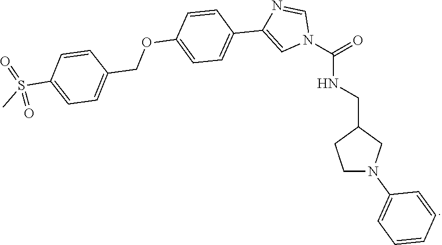 [SMILES: CS(=O)(=O)c1ccc(COc2ccc(-c3cn(C(=O)NCC4CCN(c5ccccc5)C4)cn3)cc2)cc1]

In embodiments, the compound is

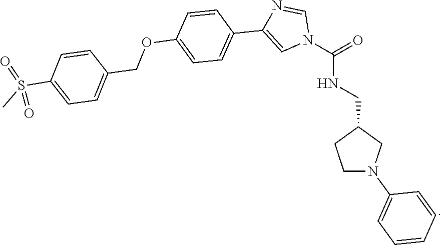 [SMILES: CS(=O)(=O)c1ccc(COc2ccc(-c3cn(C(=O)NC[C@H]4CCN(c5ccccc5)C4)cn3)cc2)cc1]

In embodiments, the compound is

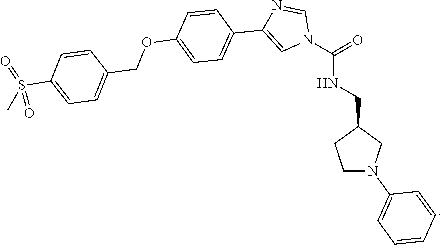 [SMILES: CS(=O)(=O)c1ccc(COc2ccc(-c3cn(C(=O)NC[C@@H]4CCN(c5ccccc5)C4)cn3)cc2)cc1]

In embodiments, the compound is

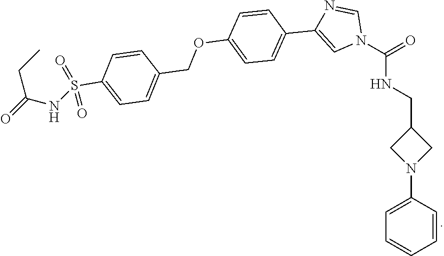 [SMILES: CCC(=O)NS(=O)(=O)c1ccc(COc2ccc(-c3cn(C(=O)NCC4CN(c5ccccc5)C4)cn3)cc2)cc1]

In embodiments, the compound is

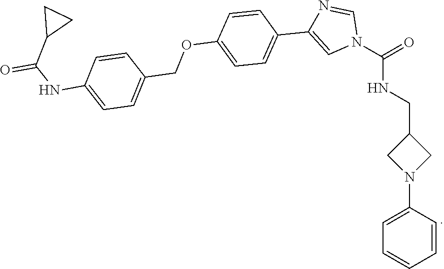 [SMILES: O=C(Nc1ccc(COc2ccc(-c3cn(C(=O)NCC4CN(c5ccccc5)C4)cn3)cc2)cc1)C1CC1]

In embodiments, the compound is

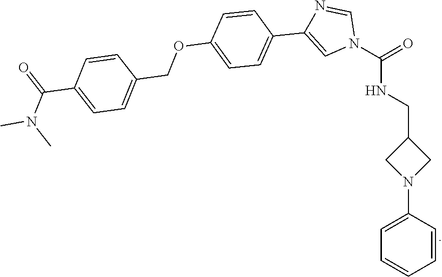 [SMILES: CN(C)C(=O)c1ccc(COc2ccc(-c3cn(C(=O)NCC4CN(c5ccccc5)C4)cn3)cc2)cc1]

In embodiments, the compound is

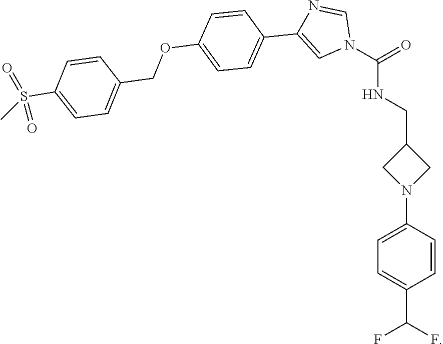 [SMILES: CS(=O)(=O)c1ccc(COc2ccc(-c3cn(C(=O)NCC4CN(c5ccc(C(F)F)cc5)C4)cn3)cc2)cc1]

In embodiments, the compound is

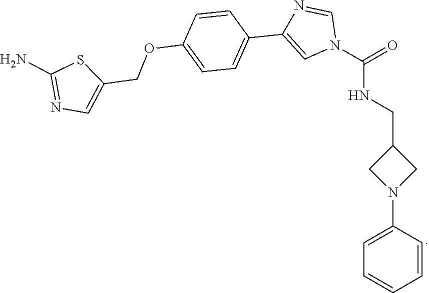 [SMILES: Nc1ncc(COc2ccc(-c3cn(C(=O)NCC4CN(c5ccccc5)C4)cn3)cc2)s1]

In embodiments, the compound is

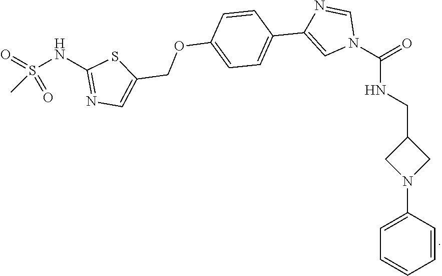 [SMILES: CS(=O)(=O)Nc1ncc(COc2ccc(-c3cn(C(=O)NCC4CN(c5ccccc5)C4)cn3)cc2)s1]

In embodiments, the compound is

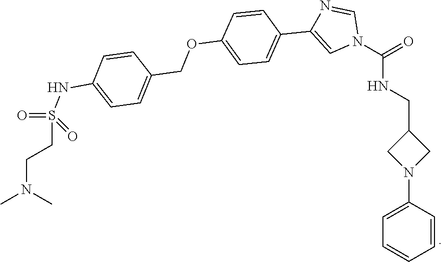 [SMILES: CN(C)CCS(=O)(=O)Nc1ccc(COc2ccc(-c3cn(C(=O)NCC4CN(c5ccccc5)C4)cn3)cc2)cc1]

In embodiments, the compound is

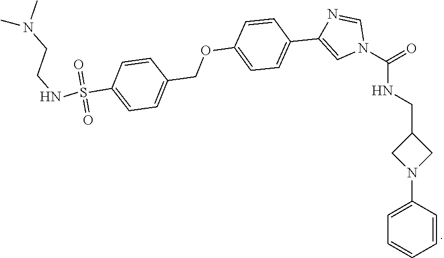 [SMILES: CN(C)CCNS(=O)(=O)c1ccc(COc2ccc(-c3cn(C(=O)NCC4CN(c5ccccc5)C4)cn3)cc2)cc1]

In embodiments, the compound is

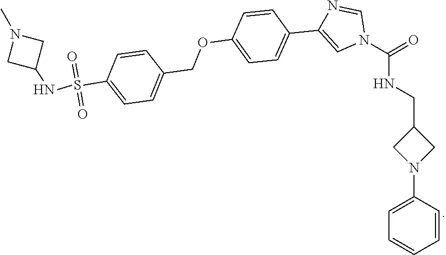 [SMILES: CN1CC(NS(=O)(=O)c2ccc(COc3ccc(-c4cn(C(=O)NCC5CN(c6ccccc6)C5)cn4)cc3)cc2)C1]

In embodiments, the compound is

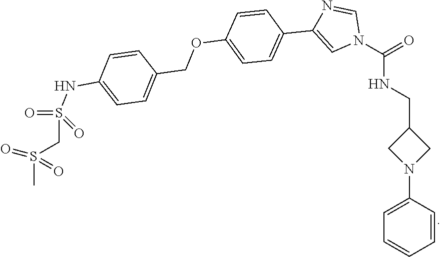 [SMILES: CS(=O)(=O)CS(=O)(=O)Nc1ccc(COc2ccc(-c3cn(C(=O)NCC4CN(c5ccccc5)C4)cn3)cc2)cc1]

In embodiments, the compound is

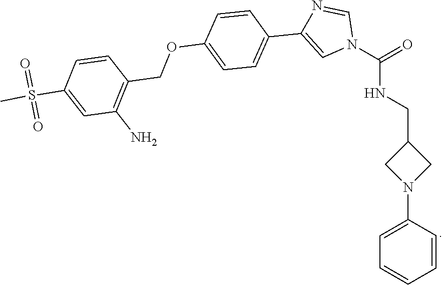 [SMILES: CS(=O)(=O)c1ccc(COc2ccc(-c3cn(C(=O)NCC4CN(c5ccccc5)C4)cn3)cc2)c(N)c1]

In embodiments, the compound is

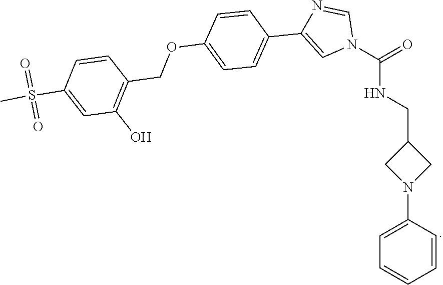 [SMILES: CS(=O)(=O)c1ccc(COc2ccc(-c3cn(C(=O)NCC4CN(c5ccccc5)C4)cn3)cc2)c(O)c1]

In embodiments, the compound is

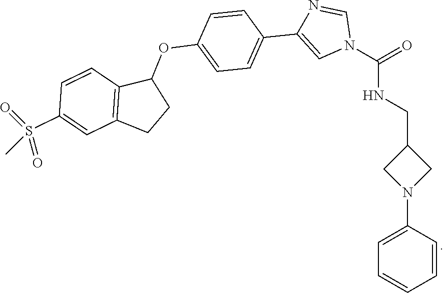 [SMILES: CS(=O)(=O)c1ccc2c(c1)CCC2Oc1ccc(-c2cn(C(=O)NCC3CN(c4ccccc4)C3)cn2)cc1]

In embodiments, the compound is

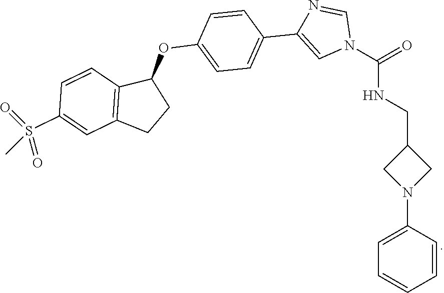 [SMILES: CS(=O)(=O)c1ccc2c(c1)CC[C@@H]2Oc1ccc(-c2cn(C(=O)NCC3CN(c4ccccc4)C3)cn2)cc1]

In embodiments, the compound is

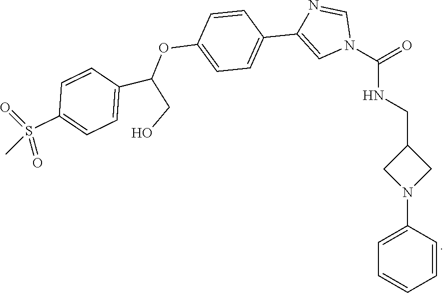 [SMILES: CS(=O)(=O)c1ccc(C(CO)Oc2ccc(-c3cn(C(=O)NCC4CN(c5ccccc5)C4)cn3)cc2)cc1]

In embodiments, the compound is

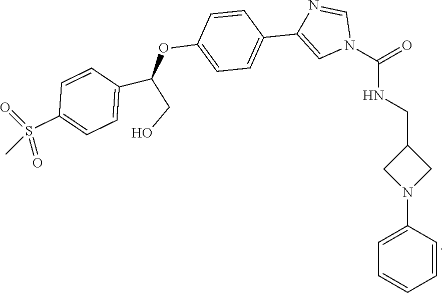 [SMILES: CS(=O)(=O)c1ccc([C@H](CO)Oc2ccc(-c3cn(C(=O)NCC4CN(c5ccccc5)C4)cn3)cc2)cc1]

In embodiments, the compound is

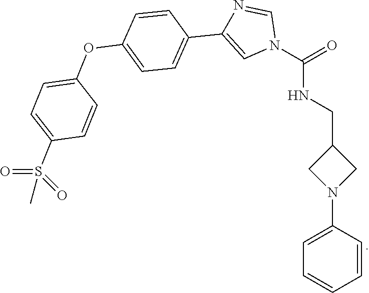 [SMILES: CS(=O)(=O)c1ccc(Oc2ccc(-c3cn(C(=O)NCC4CN(c5ccccc5)C4)cn3)cc2)cc1]

In embodiments, the compound is

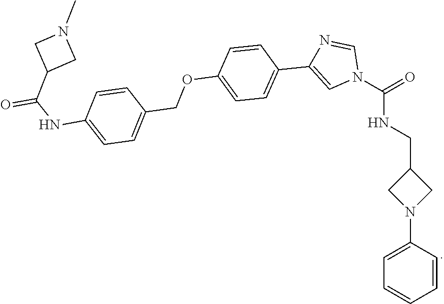 [SMILES: CN1CC(C(=O)Nc2ccc(COc3ccc(-c4cn(C(=O)NCC5CN(c6ccccc6)C5)cn4)cc3)cc2)C1]

In embodiments, the compound is

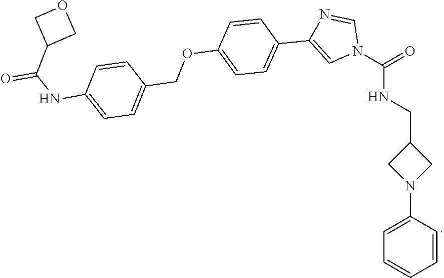 [SMILES: O=C(Nc1ccc(COc2ccc(-c3cn(C(=O)NCC4CN(c5ccccc5)C4)cn3)cc2)cc1)C1COC1]

In embodiments, the compound is

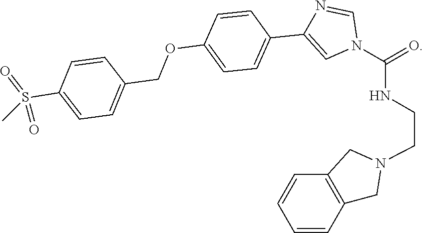 [SMILES: CS(=O)(=O)c1ccc(COc2ccc(-c3cn(C(=O)NCCN4Cc5ccccc5C4)cn3)cc2)cc1]

In embodiments, the compound is

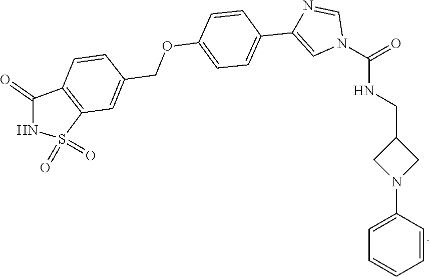 [SMILES: O=C1NS(=O)(=O)c2cc(COc3ccc(-c4cn(C(=O)NCC5CN(c6ccccc6)C5)cn4)cc3)ccc21]

In embodiments, the compound is

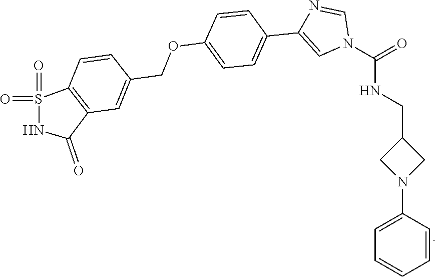 [SMILES: O=C1NS(=O)(=O)c2ccc(COc3ccc(-c4cn(C(=O)NCC5CN(c6ccccc6)C5)cn4)cc3)cc21]

In embodiments, the compound is

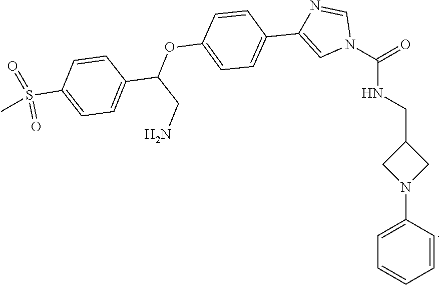 [SMILES: CS(=O)(=O)c1ccc(C(CN)Oc2ccc(-c3cn(C(=O)NCC4CN(c5ccccc5)C4)cn3)cc2)cc1]

In embodiments, the compound is

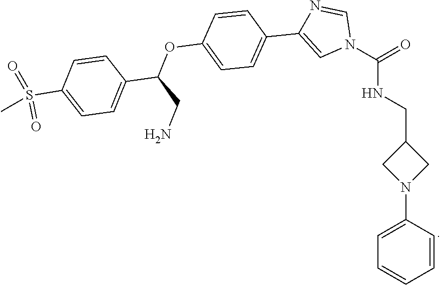 [SMILES: CS(=O)(=O)c1ccc([C@H](CN)Oc2ccc(-c3cn(C(=O)NCC4CN(c5ccccc5)C4)cn3)cc2)cc1]

In embodiments, the compound is

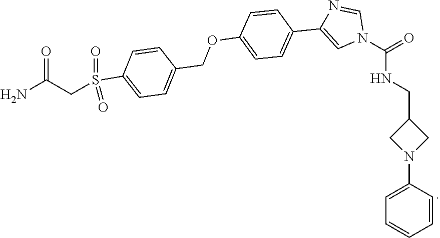 [SMILES: NC(=O)CS(=O)(=O)c1ccc(COc2ccc(-c3cn(C(=O)NCC4CN(c5ccccc5)C4)cn3)cc2)cc1]

In embodiments, the compound is

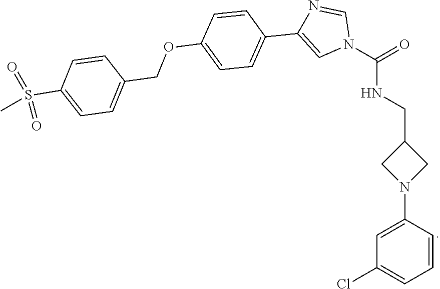 [SMILES: CS(=O)(=O)c1ccc(COc2ccc(-c3cn(C(=O)NCC4CN(c5cccc(Cl)c5)C4)cn3)cc2)cc1]

In embodiments, the compound is

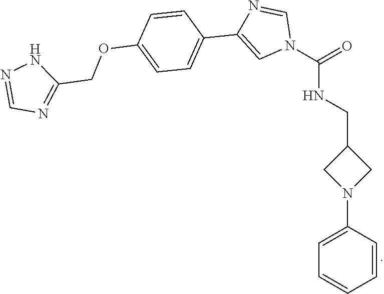 [SMILES: O=C(NCC1CN(c2ccccc2)C1)n1cnc(-c2ccc(OCc3ncn[nH]3)cc2)c1]

In embodiments, the compound is

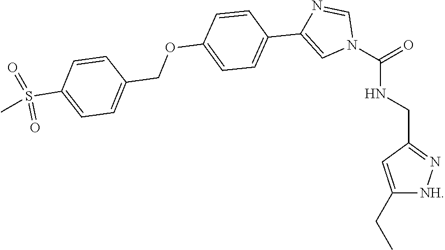 [SMILES: CCc1cc(CNC(=O)n2cnc(-c3ccc(OCc4ccc(S(C)(=O)=O)cc4)cc3)c2)n[nH]1]

In embodiments, the compound is

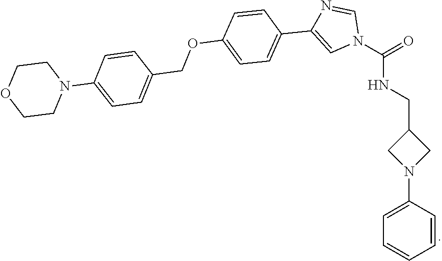 [SMILES: O=C(NCC1CN(c2ccccc2)C1)n1cnc(-c2ccc(OCc3ccc(N4CCOCC4)cc3)cc2)c1]

In embodiments, the compound is

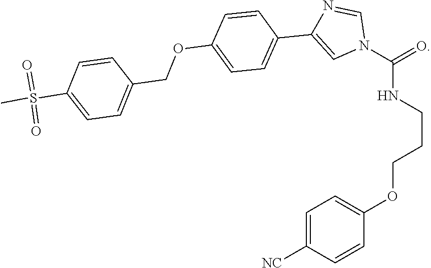 [SMILES: CS(=O)(=O)c1ccc(COc2ccc(-c3cn(C(=O)NCCCOc4ccc(C#N)cc4)cn3)cc2)cc1]

In embodiments, the compound is

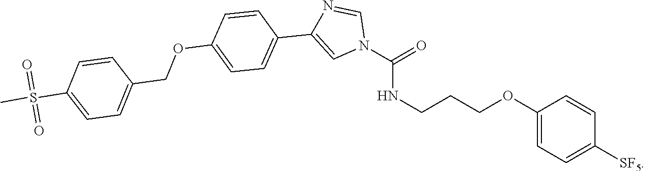

In embodiments, the compound is

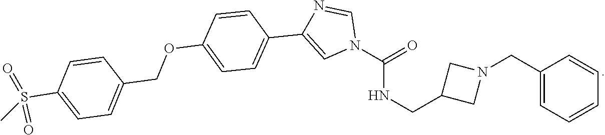 [SMILES: CS(=O)(=O)c1ccc(COc2ccc(-c3cn(C(=O)NCC4CN(Cc5ccccc5)C4)cn3)cc2)cc1]

In embodiments, the compound is

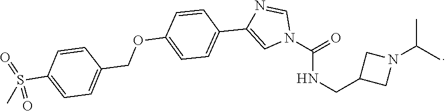 [SMILES: CC(C)N1CC(CNC(=O)n2cnc(-c3ccc(OCc4ccc(S(C)(=O)=O)cc4)cc3)c2)C1]

In embodiments, the compound is

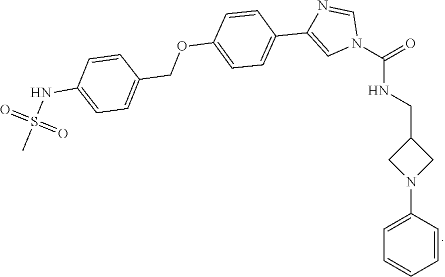 [SMILES: CS(=O)(=O)Nc1ccc(COc2ccc(-c3cn(C(=O)NCC4CN(c5ccccc5)C4)cn3)cc2)cc1]

In embodiments, the compound is

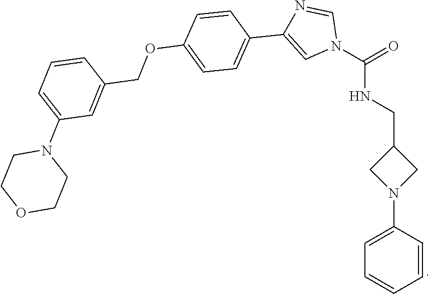 [SMILES: O=C(NCC1CN(c2ccccc2)C1)n1cnc(-c2ccc(OCc3cccc(N4CCOCC4)c3)cc2)c1]

In embodiments, the compound is

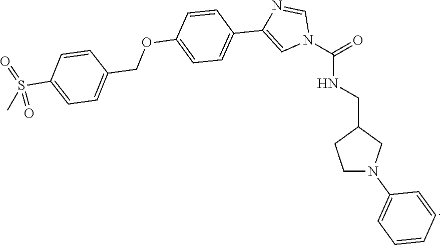 [SMILES: CS(=O)(=O)c1ccc(COc2ccc(-c3cn(C(=O)NCC4CCN(c5ccccc5)C4)cn3)cc2)cc1]

In embodiments, the compound is

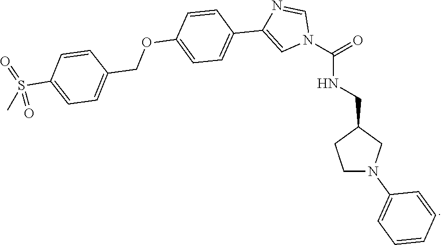 [SMILES: CS(=O)(=O)c1ccc(COc2ccc(-c3cn(C(=O)NC[C@@H]4CCN(c5ccccc5)C4)cn3)cc2)cc1]

In embodiments, the compound is

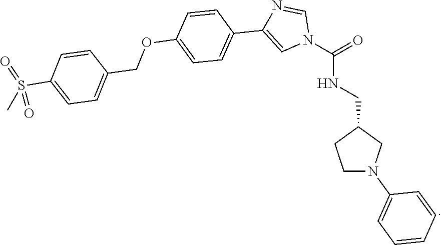 [SMILES: CS(=O)(=O)c1ccc(COc2ccc(-c3cn(C(=O)NC[C@H]4CCN(c5ccccc5)C4)cn3)cc2)cc1]

In embodiments, the compound is

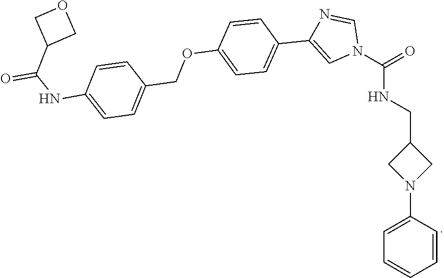 [SMILES: O=C(Nc1ccc(COc2ccc(-c3cn(C(=O)NCC4CN(c5ccccc5)C4)cn3)cc2)cc1)C1COC1]

In embodiments, the compound is

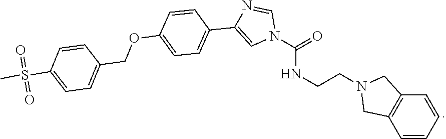 [SMILES: CS(=O)(=O)c1ccc(COc2ccc(-c3cn(C(=O)NCCN4Cc5ccccc5C4)cn3)cc2)cc1]

In embodiments, the compound is

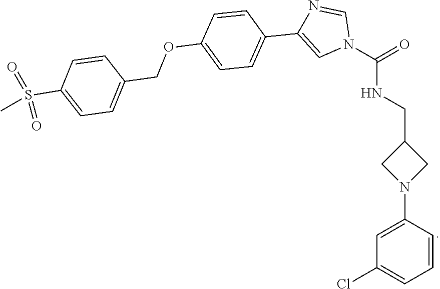 [SMILES: CS(=O)(=O)c1ccc(COc2ccc(-c3cn(C(=O)NCC4CN(c5cccc(Cl)c5)C4)cn3)cc2)cc1]

In embodiments, the compound is

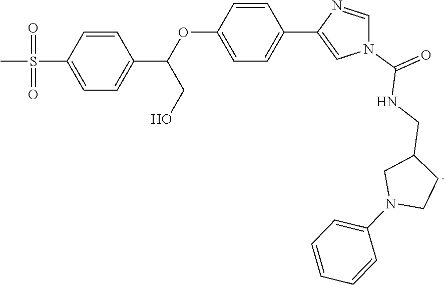 [SMILES: CS(=O)(=O)c1ccc(C(CO)Oc2ccc(-c3cn(C(=O)NCC4CCN(c5ccccc5)C4)cn3)cc2)cc1]

In embodiments, the compound is

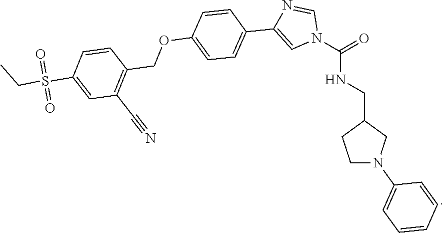 [SMILES: CCS(=O)(=O)c1ccc(COc2ccc(-c3cn(C(=O)NCC4CCN(c5ccccc5)C4)cn3)cc2)c(C#N)c1]

In embodiments, the compound is

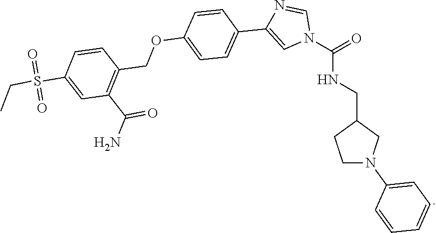 [SMILES: CCS(=O)(=O)c1ccc(COc2ccc(-c3cn(C(=O)NCC4CCN(c5ccccc5)C4)cn3)cc2)c(C(N)=O)c1]

In embodiments, the compound is

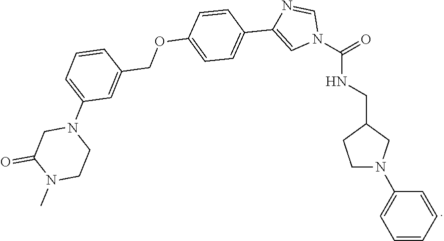 [SMILES: CN1CCN(c2cccc(COc3ccc(-c4cn(C(=O)NCC5CCN(c6ccccc6)C5)cn4)cc3)c2)CC1=O]

In embodiments, the compound is

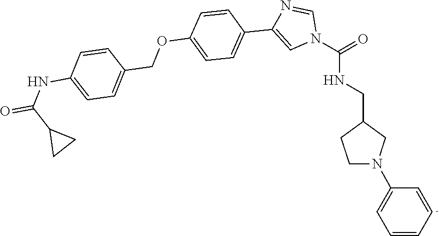 [SMILES: O=C(Nc1ccc(COc2ccc(-c3cn(C(=O)NCC4CCN(c5ccccc5)C4)cn3)cc2)cc1)C1CC1]

In embodiments, the compound is

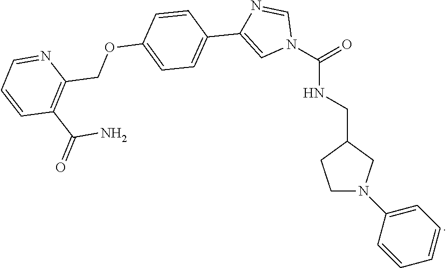 [SMILES: NC(=O)c1cccnc1COc1ccc(-c2cn(C(=O)NCC3CCN(c4ccccc4)C3)cn2)cc1]

In embodiments, the compound is

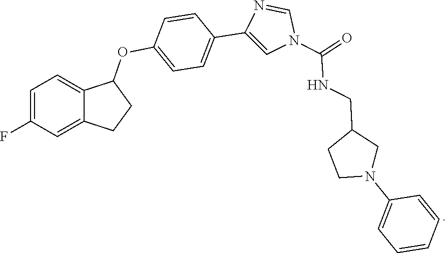 [SMILES: O=C(NCC1CCN(c2ccccc2)C1)n1cnc(-c2ccc(OC3CCc4cc(F)ccc43)cc2)c1]

In embodiments, the compound is

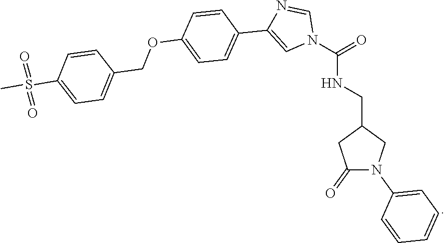 [SMILES: CS(=O)(=O)c1ccc(COc2ccc(-c3cn(C(=O)NCC4CC(=O)N(c5ccccc5)C4)cn3)cc2)cc1]

In embodiments, the compound is

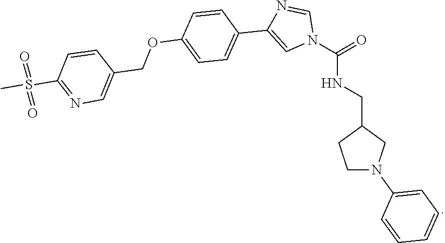 [SMILES: CS(=O)(=O)c1ccc(COc2ccc(-c3cn(C(=O)NCC4CCN(c5ccccc5)C4)cn3)cc2)cn1]

In embodiments, the compound is

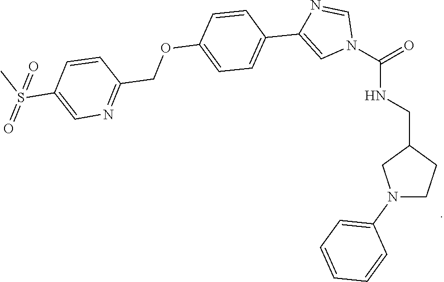 [SMILES: CS(=O)(=O)c1ccc(COc2ccc(-c3cn(C(=O)NCC4CCN(c5ccccc5)C4)cn3)cc2)nc1]

In embodiments, the compound is

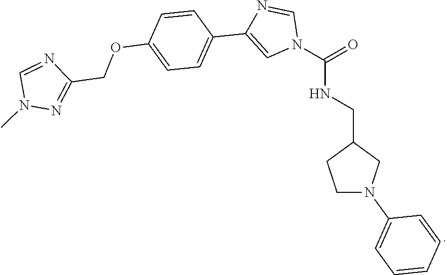 [SMILES: Cn1cnc(COc2ccc(-c3cn(C(=O)NCC4CCN(c5ccccc5)C4)cn3)cc2)n1]

In embodiments, the compound is

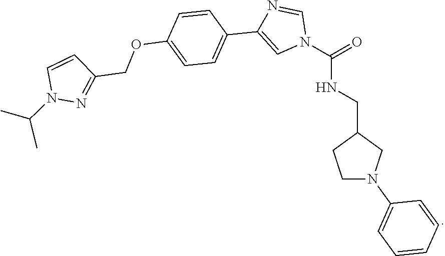 [SMILES: CC(C)n1ccc(COc2ccc(-c3cn(C(=O)NCC4CCN(c5ccccc5)C4)cn3)cc2)n1]

In embodiments, the compound is

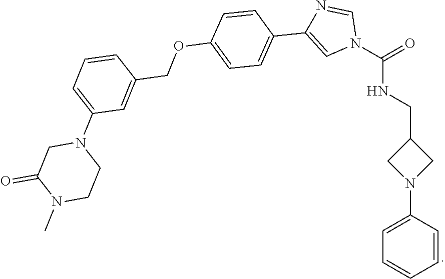 [SMILES: CN1CCN(c2cccc(COc3ccc(-c4cn(C(=O)NCC5CN(c6ccccc6)C5)cn4)cc3)c2)CC1=O]

In embodiments, the compound is

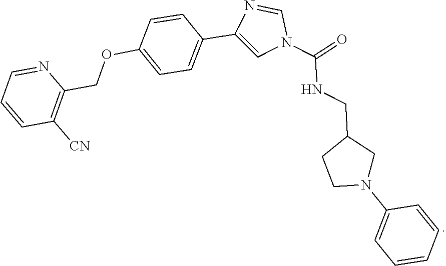 [SMILES: N#Cc1cccnc1COc1ccc(-c2cn(C(=O)NCC3CCN(c4ccccc4)C3)cn2)cc1]

In embodiments, the compound is

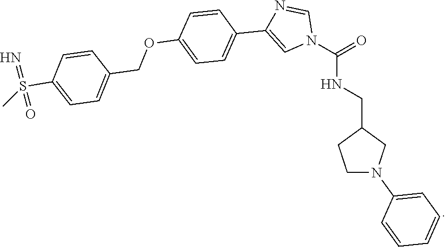 [SMILES: CS(=N)(=O)c1ccc(COc2ccc(-c3cn(C(=O)NCC4CCN(c5ccccc5)C4)cn3)cc2)cc1]

In embodiments, the compound is

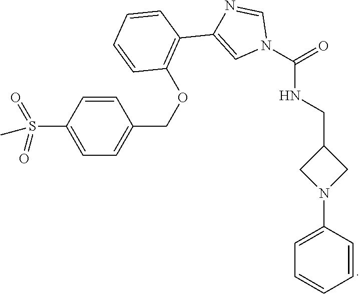 [SMILES: CS(=O)(=O)c1ccc(COc2ccccc2-c2cn(C(=O)NCC3CN(c4ccccc4)C3)cn2)cc1]

In embodiments, the compound is

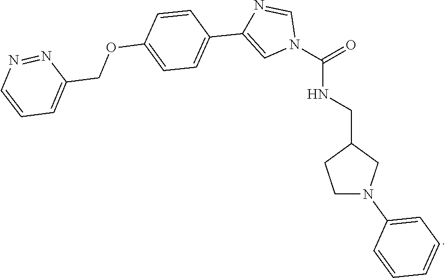 [SMILES: O=C(NCC1CCN(c2ccccc2)C1)n1cnc(-c2ccc(OCc3cccnn3)cc2)c1]

In embodiments, the compound is

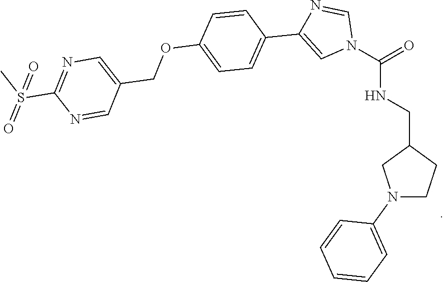 [SMILES: CS(=O)(=O)c1ncc(COc2ccc(-c3cn(C(=O)NCC4CCN(c5ccccc5)C4)cn3)cc2)cn1]

In embodiments, the compound is

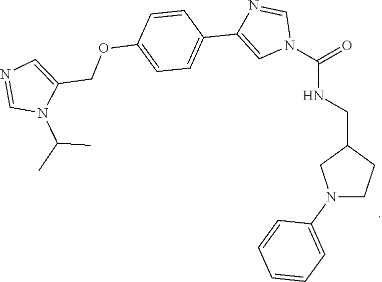 [SMILES: CC(C)n1cncc1COc1ccc(-c2cn(C(=O)NCC3CCN(c4ccccc4)C3)cn2)cc1]

In embodiments, the compound is

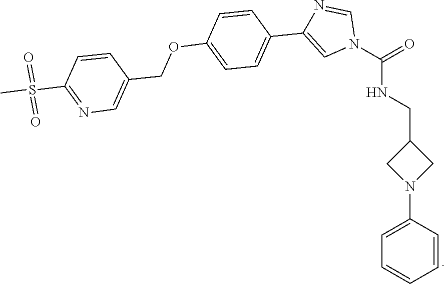 [SMILES: CS(=O)(=O)c1ccc(COc2ccc(-c3cn(C(=O)NCC4CN(c5ccccc5)C4)cn3)cc2)cn1]

In embodiments, the compound is

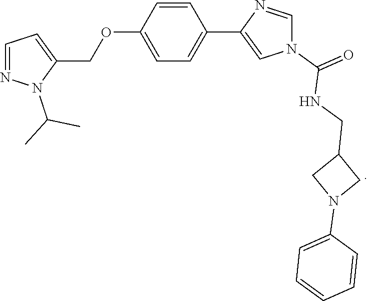 [SMILES: CC(C)n1nccc1COc1ccc(-c2cn(C(=O)NCC3CN(c4ccccc4)C3)cn2)cc1]

In embodiments, the compound is

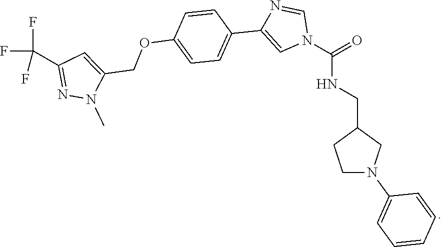 [SMILES: Cn1nc(C(F)(F)F)cc1COc1ccc(-c2cn(C(=O)NCC3CCN(c4ccccc4)C3)cn2)cc1]

In embodiments, the compound is

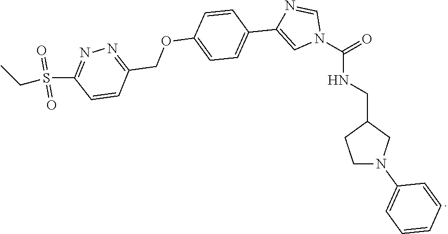 [SMILES: CCS(=O)(=O)c1ccc(COc2ccc(-c3cn(C(=O)NCC4CCN(c5ccccc5)C4)cn3)cc2)nn1]

In embodiments, the compound is

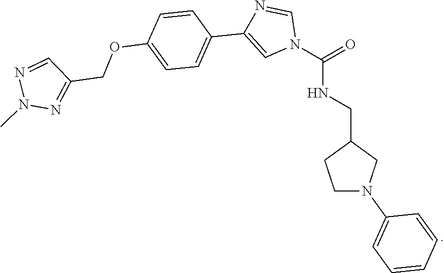 [SMILES: Cn1ncc(COc2ccc(-c3cn(C(=O)NCC4CCN(c5ccccc5)C4)cn3)cc2)n1]

In embodiments, the compound is

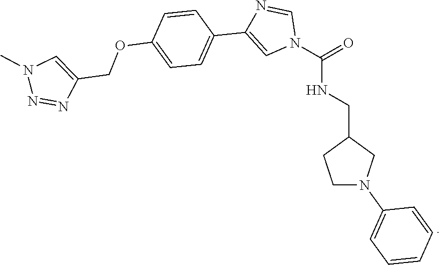 [SMILES: Cn1cc(COc2ccc(-c3cn(C(=O)NCC4CCN(c5ccccc5)C4)cn3)cc2)nn1]

In embodiments, the compound is

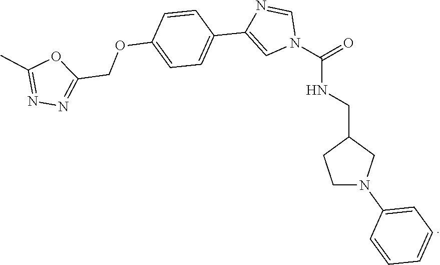 [SMILES: Cc1nnc(COc2ccc(-c3cn(C(=O)NCC4CCN(c5ccccc5)C4)cn3)cc2)o1]

In embodiments, the compound is

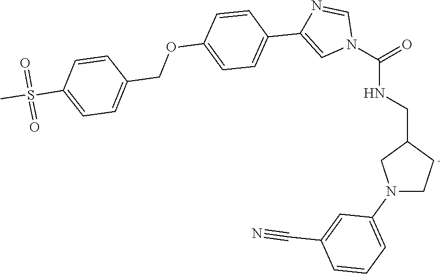 [SMILES: CS(=O)(=O)c1ccc(COc2ccc(-c3cn(C(=O)NCC4CCN(c5cccc(C#N)c5)C4)cn3)cc2)cc1]

In embodiments, the compound is

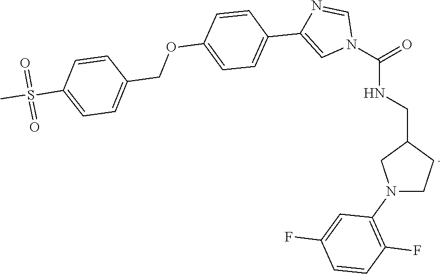 [SMILES: CS(=O)(=O)c1ccc(COc2ccc(-c3cn(C(=O)NCC4CCN(c5cc(F)ccc5F)C4)cn3)cc2)cc1]

In embodiments, the compound is

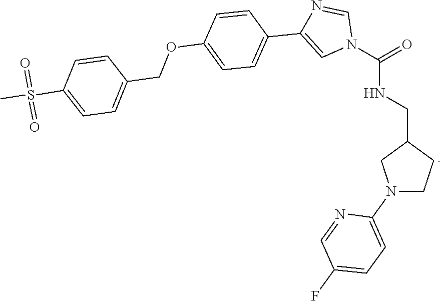 [SMILES: CS(=O)(=O)c1ccc(COc2ccc(-c3cn(C(=O)NCC4CCN(c5ccc(F)cn5)C4)cn3)cc2)cc1]

In embodiments, the compound is

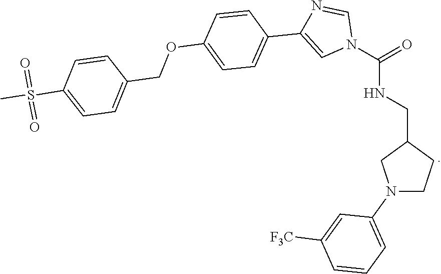 [SMILES: CS(=O)(=O)c1ccc(COc2ccc(-c3cn(C(=O)NCC4CCN(c5cccc(C(F)(F)F)c5)C4)cn3)cc2)cc1]

In embodiments, the compound is

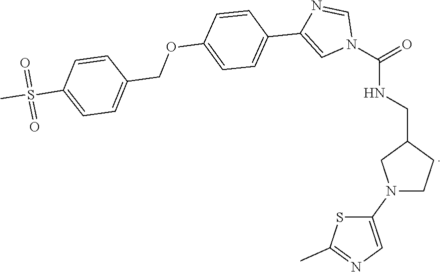 [SMILES: Cc1ncc(N2CCC(CNC(=O)n3cnc(-c4ccc(OCc5ccc(S(C)(=O)=O)cc5)cc4)c3)C2)s1]

In embodiments, the compound is

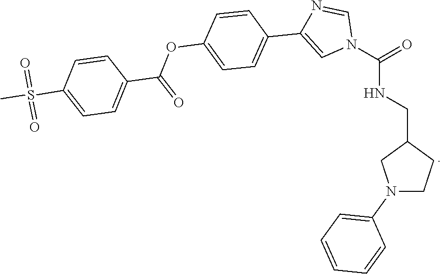 [SMILES: CS(=O)(=O)c1ccc(C(=O)Oc2ccc(-c3cn(C(=O)NCC4CCN(c5ccccc5)C4)cn3)cc2)cc1]

In embodiments, the compound is

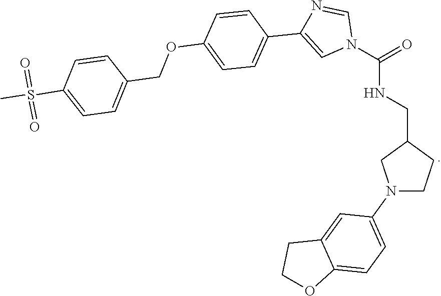 [SMILES: CS(=O)(=O)c1ccc(COc2ccc(-c3cn(C(=O)NCC4CCN(c5ccc6c(c5)CCO6)C4)cn3)cc2)cc1]

In embodiments, the compound is

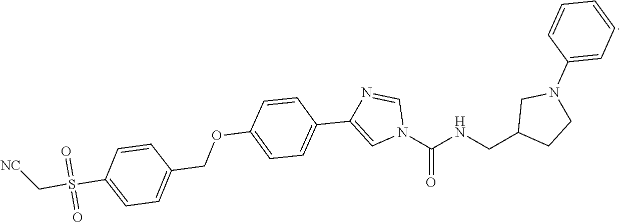 [SMILES: N#CCS(=O)(=O)c1ccc(COc2ccc(-c3cn(C(=O)NCC4CCN(c5ccccc5)C4)cn3)cc2)cc1]

In embodiments, the compound is useful as a comparator compound. In embodiments, the comparator compound can be used to assess the activity of a test compound as set forth in an assay described herein (e.g., in the examples section, FIGURES, or tables).

In embodiments, the compound is a compound as described herein, including in embodiments. In embodiments the compound is a compound described herein (e.g., in the examples section, FIGURES, tables, or claims).

In embodiments, R² is not —OR^{2D}, —SR^{2D}, substituted or unsubstituted alkyl, or substituted or unsubstituted aryl; wherein R^{2D} is as described herein, including in embodiments. In embodiments, R² is not —OR^{2D}. In embodiments, R² is not —SR^{2D}. In embodiments, R² is not —SCH₃. In embodiments, R² is not substituted or unsubstituted alkyl. In embodiments, R² is not substituted or unsubstituted C₁-C₆ alkyl. In embodiments, R² is not unsubstituted methyl. In embodiments, R² is not unsubstituted ethyl. In embodiments, R² is not unsubstituted propyl. In embodiments, R² is not unsubstituted n-propyl. In embodiments, R² is not unsubstituted isopropyl. In embodiments, R² is not unsubstituted butyl. In embodiments, R² is not unsubstituted n-butyl. In embodiments, R² is not unsubstituted isobutyl. In embodiments, R² is not unsubstituted tert-butyl. In embodiments, R² is not unsubstituted pentyl. In embodiments, R² is not unsubstituted hexyl. In embodiments, R² is not substituted or unsubstituted aryl. In embodiments, R² is not substituted or unsubstituted phenyl.

In embodiments, R^{2D} is not substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R^{2D} is not substituted or unsubstituted C₁-C₆ alkyl, substituted or unsubstituted 2 to 6 membered heteroalkyl, substituted or unsubstituted C₃-C₈ cycloalkyl, substituted or unsubstituted 3 to 8 membered heterocycloalkyl, substituted or unsubstituted phenyl, or substituted or unsubstituted 5 to 6 membered heteroaryl. In embodiments, R^{2D} is not unsubstituted methyl. In embodiments, R^{2D} is not unsubstituted ethyl. In embodiments, R^{2D} is not unsubstituted propyl. In embodiments, R^{2D} is not unsubstituted n-propyl. In embodiments, R^{2D} is not unsubstituted isopropyl. In embodiments, R^{2D} is not unsubstituted butyl. In embodiments, R^{2D} is not unsubstituted n-butyl. In embodiments, R^{2D} is not unsubstituted isobutyl. In embodiments, R^{2D} is not unsubstituted tert-butyl. In embodiments, R^{2D} is not unsubstituted pentyl. In embodiments, R^{2D} is not unsubstituted hexyl. In embodiments, R^{2D} is not substituted or unsubstituted cyclopropyl. In embodiments, R^{2D} is not substituted or unsubstituted cyclobutyl. In embodiments, R^{2D} is not substituted or unsubstituted cyclopentyl. In embodiments, R^{2D} is not substituted or unsubstituted cyclohexyl. In embodiments, R^{2D} is not substituted or unsubstituted piperidinyl. In embodiments, R^{2D} is not substituted or unsubstituted aryl.

### III. Pharmaceutical Compositions

In an aspect is provided a pharmaceutical composition including a compound described herein, or a pharmaceutically acceptable salt thereof, and a pharmaceutically acceptable excipient.

In embodiments, the compound is a compound of formula (I), (Ia), (Ib), (Ic), (IIa), (IIb), or (IIc). In embodiments, the compound is a compound of formula (I). In embodiments, the compound is a compound of formula (Ia). In embodiments, the compound is a compound of formula (Ib). In embodiments, the compound is a compound of formula (Ic). In embodiments, the compound is a compound of formula (IIa). In embodiments, the compound is a compound of formula (IIb). In embodiments, the compound is a compound of formula (IIc).

In embodiments, the pharmaceutical composition includes an effective amount of the compound. In embodiments, the pharmaceutical composition includes a therapeutically effective amount of the compound.

### IV. Methods of Use

In an aspect is provided a method of treating fibrotic disease in a subject in need thereof, the method including administering to the subject in need thereof a therapeutically effective amount of an acid ceramidase inhibitor, or a pharmaceutically acceptable salt thereof.

In an aspect is provided a method of treating fibrotic disease in a subject in need thereof, the method including administering to the subject in need thereof a therapeutically effective amount of a compound described herein, or a pharmaceutically acceptable salt thereof.

In embodiments, the fibrotic disease is nonalcoholic steatohepatitis. In embodiments, the fibrotic disease is liver fibrosis.

In an aspect is provided a method of treating cancer in a subject in need thereof, the method including administering to the subject in need thereof a therapeutically effective amount of a compound described herein, or a pharmaceutically acceptable salt of solvate thereof.

In embodiments, the cancer is liver cancer.

### V. Embodiments

Embodiment P1. A compound, or a pharmaceutically acceptable salt thereof, having the formula:

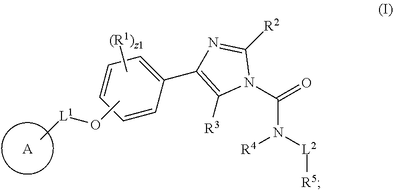

wherein
L¹ is a bond, substituted or unsubstituted alkylene, or substituted or unsubstituted heteroalkylene;
Ring A is substituted or unsubstituted cycloalkyl, substituted or unsubstituted heterocycloalkyl, substituted or unsubstituted aryl, or substituted or unsubstituted heteroaryl;
R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)OR^{1C}, —C(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R¹ substituents may optionally be joined to form a substituted or unsubstituted cycloalkyl, substituted or unsubstituted heterocycloalkyl, substituted or unsubstituted aryl, or substituted or unsubstituted heteroaryl;
z1 is an integer from 0 to 4;
R² is hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is hydrogen, halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NR^{3C}NR^{3A}R^{3B}, —ONR^{3A}R^{3B}, —NHC(O)NR^{3C}NR^{3A}R^{3B}, —NHC(O)NR^{3A}R^{3B}, —N(O)ₘ₃, —NR^{3A}R^{3B}, —C(O)R^{3C}, —C(O)OR^{3C}, —C(O)NR^{3A}R^{3B}, —OR^{3D}, —SR^{3D}, —NR^{3A}SO₂R^{3D}, —NR^{3A}C(O)R^{3C}, —NR^{3A}C(O)OR^{3C}, —NR^{3A}OR^{3C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁴ is hydrogen or unsubstituted C₁-C₄ alkyl;
L² is -L^{2A}-L^{2B}-L^{2C};
L^{2A}, L^{2B}, and L^{2C} are independently a bond, —O—, —NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R⁵ is hydrogen, halogen, —CX⁵₃, —CHX⁵₂, —CH₂X⁵, —OCX⁵₃, —OCH₂X⁵, —OCHX⁵₂, —CN, —SOₙ₅R^{5D}, —SOᵥ₅NR^{5A}R^{5B}, —NR^{5C}NR^{5A}R^{5B}, —ONR^{5A}R^{5B}, —NHC(O)NR^{5C}NR^{5A}R^{5B}, —NHC(O)NR^{5A}R^{5B}, —N(O)ₘ₅, —NR^{5A}R^{5B}, —C(O)R^{5C}, —C(O)OR^{5C}, —C(O)NR^{5A}R^{5B}, —OR^{5D}, —SR 5D, —NR^{5A}SO₂R^{5D}, —NR^{5A}C(O)R^{5C}, —NR^{5A}C(O)OR^{5C}, —NR^{5A}OR^{5C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{1A}, R^{1B}, R^{1C}, R^{1D}, R^{2A}, R^{2B}, R^{2C}, R^{2D}, R^{3A}, R^{3B}, R^{3C}, R^{3D}, R^{5A}, R^{5B}, R^{5C}, and R^{5D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{5A} and R^{5B} substituents bonded to the same nitrogen atom may optionally be joined to form a substituted or unsubstituted heterocycloalkyl or substituted or unsubstituted heteroaryl;
X¹, X², X³, and X⁵ are independently —F, —Cl, —Br, or —I;
n1, n2, n3, and n5 are independently an integer from 0 to 4; and
m1, m2, m3, m5, v1, v2, v3, and v5 are independently 1 or 2.

Embodiment P2. The compound of embodiment P1, having the formula:

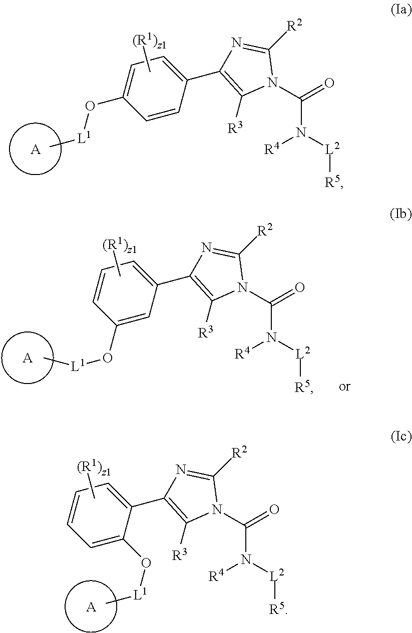

Embodiment P3. The compound of embodiment P1, having the formula:

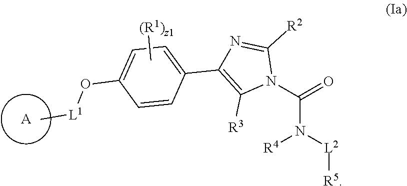

Embodiment P4. The compound of one of embodiments P1 to P3, wherein Ring A is substituted or unsubstituted 5 to 9 membered cycloalkyl, substituted or unsubstituted 5 to 6 membered heterocycloalkyl, substituted or unsubstituted phenyl, or substituted or unsubstituted 5 to 9 membered heteroaryl.

Embodiment P5. The compound of embodiment P1, having the formula:

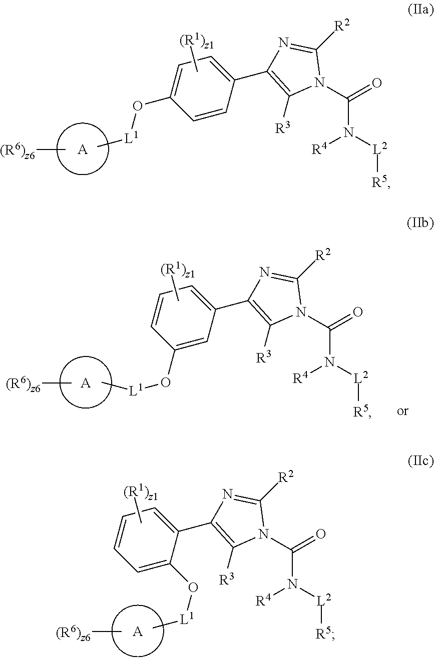

wherein
Ring A is cycloalkyl, heterocycloalkyl, aryl, or heteroaryl;
R⁶ is independently oxo, halogen, —CX⁶₃, —CHX⁶₂, —CH₂X⁶, —OCX⁶₃, —OCH₂X⁶, —OCHX⁶₂, —CN, —SOₙ₆R^{6D}, —S(O)(NH)R^{6D}, —SOᵥ₆NR^{6A}R^{6B}, —ONR^{6A}R^{6B}, —NHC(O)NR⁶CNR^{6A}R^{6B}, —NHC(O)NR^{6A}R^{6B}, —N(O)ₘ₆, —NR^{6A}R^{6B}, —C(O)R^{6C}, —C(O)OR^{6C}, —C(O)NR^{6A}R^{6B}, —OR^{6D}, —SR^{6D}, —NR^{6A}SO₂R^{6D}, —NR^{6A}C(O)R^{6C}, —NR^{6A}C(O)OR^{6C}, —NR^{6A}OR^{6C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R⁶ substituents may optionally be joined to form a substituted or unsubstituted cycloalkyl, substituted or unsubstituted heterocycloalkyl, substituted or unsubstituted aryl, or substituted or unsubstituted heteroaryl;
R^{6A}, R^{6B}, R^{6C}, and R^{6D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{6A} and R^{6B} substituents bonded to the same nitrogen atom may optionally be joined to form a substituted or unsubstituted heterocycloalkyl or substituted or unsubstituted heteroaryl;
X⁶ is independently —F, —Cl, —Br, or —I;
n6 is independently an integer from 0 to 4;
m6 and v6 are independently 1 or 2; and
z6 is an integer from 0 to 11.

Embodiment P6. The compound of embodiment P5, having the formula:

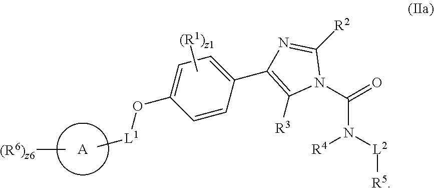

Embodiment P7. The compound of one of embodiments P5 to P6, wherein

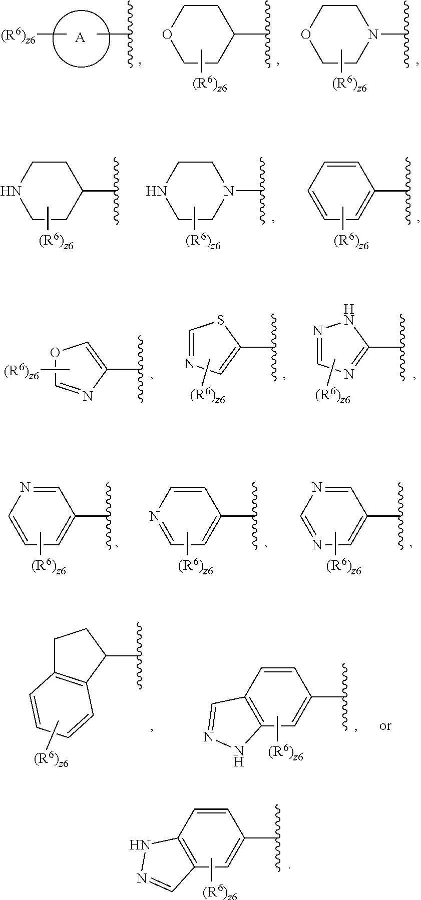

Embodiment P8. The compound of one of embodiments P5 to P7, wherein R⁶ is independently halogen, —CN, —SOₙ₆R^{6D}, —S(O)(NH)R^{6D}, —SOᵥ₆NR^{6A}R^{6B}, —NR^{6A}R^{6B}, —C(O)NR^{6A}R^{6B}, —OR^{6D}, —SR^{6D}, —NR^{6A}SO₂R^{6D}, —NR^{6A}C(O)R^{6C}, substituted or unsubstituted C₁-C₆ alkyl, substituted or unsubstituted 2 to 6 membered heteroalkyl, or substituted or unsubstituted 3 to 6 membered heterocycloalkyl.

Embodiment P9. The compound of one of embodiments P5 to P8, wherein R^{6A} and R^{6B} are independently hydrogen, substituted or unsubstituted C₁-C₄ alkyl, substituted or unsubstituted 2 to 6 membered heteroalkyl, or substituted or unsubstituted 3 to 6 membered heterocycloalkyl.

Embodiment P10. The compound of one of embodiments P5 to P8, wherein R^{6A} and R^{6B} substituents bonded to the same nitrogen atom are joined to form a substituted or unsubstituted 3 to 6 membered heterocycloalkyl.

Embodiment P11. The compound of one of embodiments P5 to P8, wherein R^{6C} is independently unsubstituted C₃-C₆ cycloalkyl or substituted or unsubstituted 3 to 6 membered heterocycloalkyl.

Embodiment P12. The compound of one of embodiments P5 to P8, wherein R^{6D} is independently hydrogen, —CHF₂, substituted or unsubstituted C₁-C₄ alkyl, unsubstituted 2 to 6 membered heteroalkyl, or substituted or unsubstituted 3 to 6 membered heterocycloalkyl.

Embodiment P13. The compound of one of embodiments P5 to P7, wherein R⁶ is independently —F, —CN, —SO₂CH₃, —SO₂NH₂, —SO₂NHCH₃, —S(O)CH₃, —S(O)(NH CH₃—NH₂, —C(O)NH₂, —SCH₃, —OH, —NHSO₂CH₃, —NHSO₂CHF₂, —CH₃,

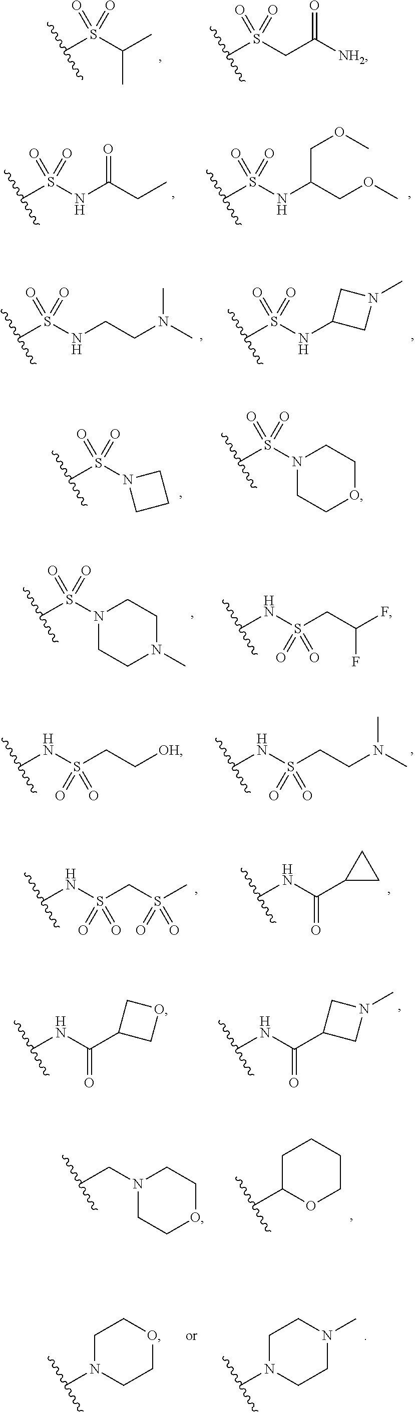

Embodiment P14. The compound of one of embodiments P5 to P7, wherein z6 is 0.

Embodiment P15. The compound of one of embodiments P5 to P13, wherein z6 is 1.

Embodiment P16. The compound of one of embodiments P5 to P13, wherein z6 is 2.

Embodiment P17. The compound of one of embodiments P5 to P7, wherein

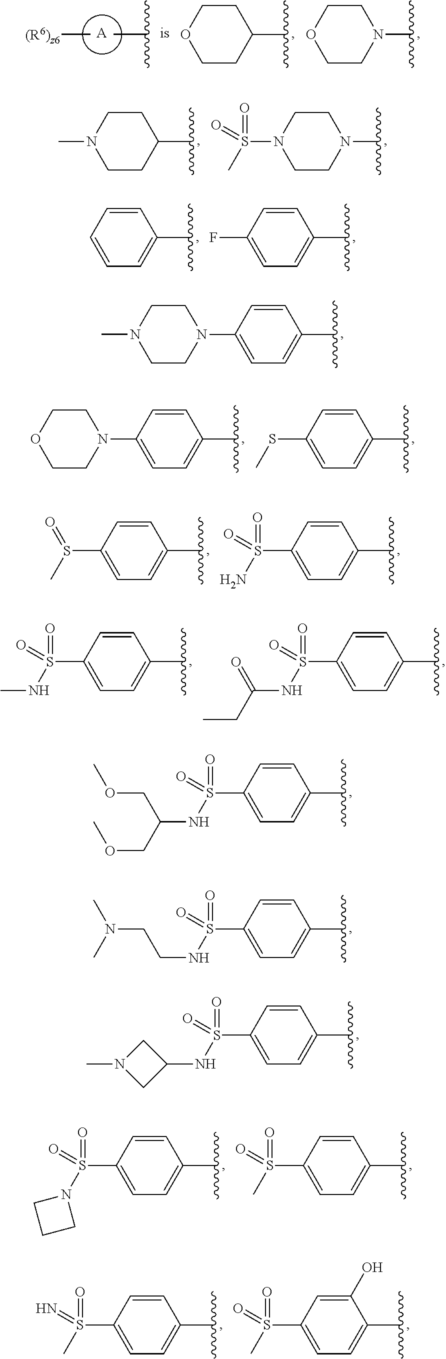

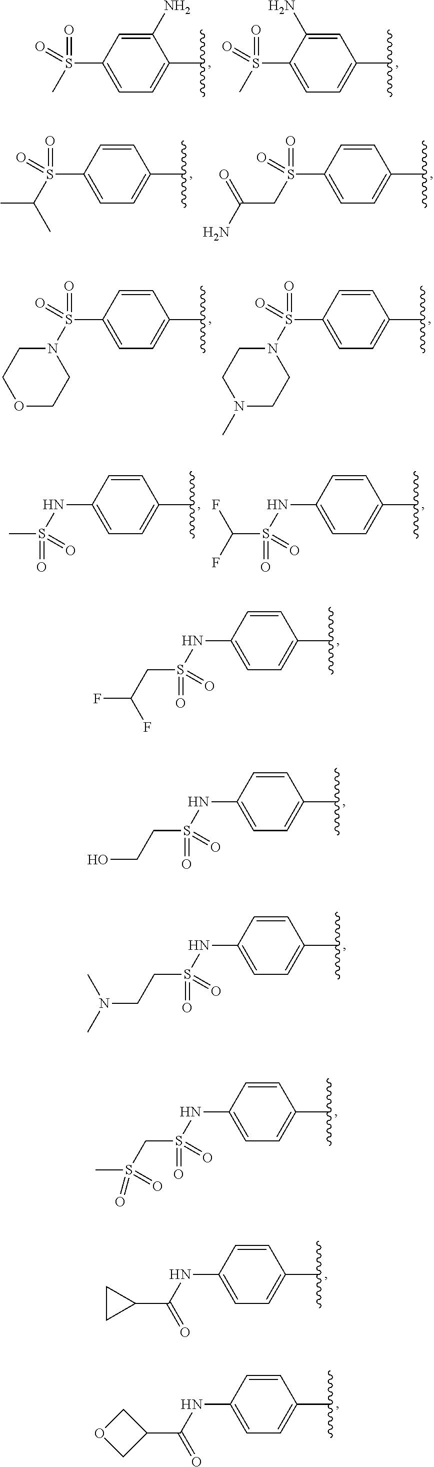

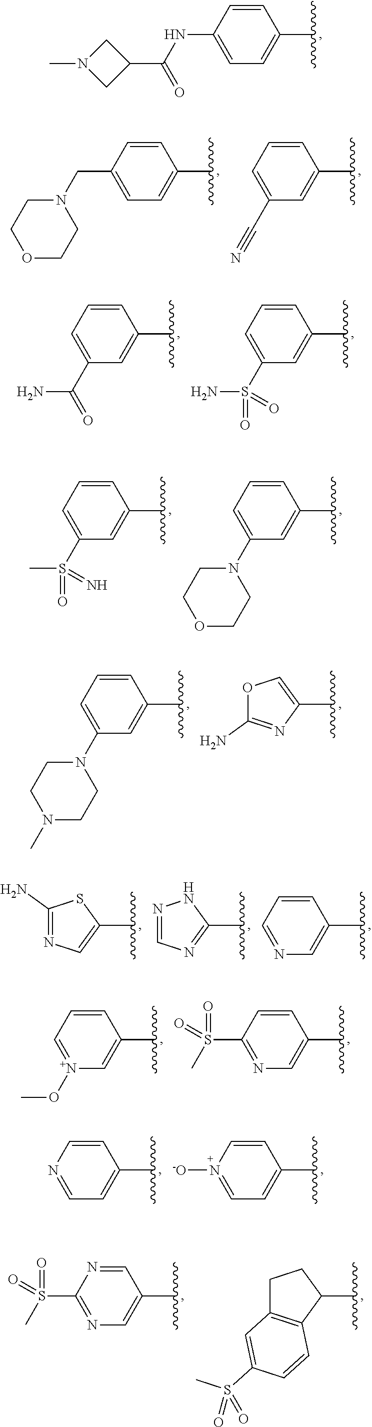

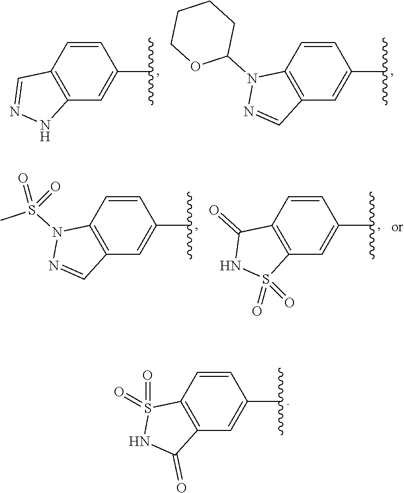

Embodiment P18. The compound of one of embodiments P1 to P17, wherein L¹ is a bond, substituted or unsubstituted C₁-C₄ alkylene, or substituted or unsubstituted 2 to 4 membered heteroalkylene.

Embodiment P19. The compound of one of embodiments P1 to P17, wherein L¹ is a bond,

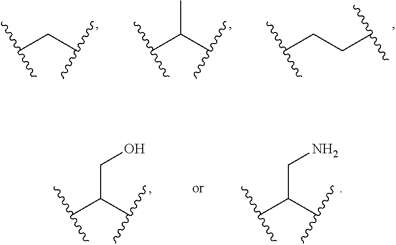

Embodiment P20. The compound of one of embodiments P1 to P19, wherein R¹ is independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment P21. The compound of one of embodiments P1 to P19, wherein z1 is 0.

Embodiment P22. The compound of one of embodiments P1 to P21, wherein R² is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment P23. The compound of one of embodiments P1 to P21, wherein R² is hydrogen or halogen.

Embodiment P24. The compound of one of embodiments P1 to P23, wherein R³ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment P25. The compound of one of embodiments P1 to P23, wherein R³ is hydrogen or halogen.

Embodiment P26. The compound of one of embodiments P1 to P25, wherein R⁴ is hydrogen or unsubstituted methyl.

Embodiment P27. The compound of one of embodiments P1 to P25, wherein R⁴ is hydrogen.

Embodiment P28. The compound of one of embodiments P1 to P27, wherein L^{2A} is unsubstituted C₁-C₆ alkylene.

Embodiment P29. The compound of one of embodiments P1 to P28, wherein L^{2B} is a bond, —O—, —NH—, unsubstituted C₁-C₆ alkylene, unsubstituted C₃-C₆ cycloalkylene, or unsubstituted 3 to 6 membered heterocycloalkylene.

Embodiment P30. The compound of one of embodiments P1 to P29, wherein L²c is a bond, —O—, —NH—, unsubstituted C₁-C₆ alkylene, unsubstituted phenylene, or unsubstituted 5 to 6 membered heteroarylene.

Embodiment P31. The compound of one of embodiments P1 to P27, wherein L² is

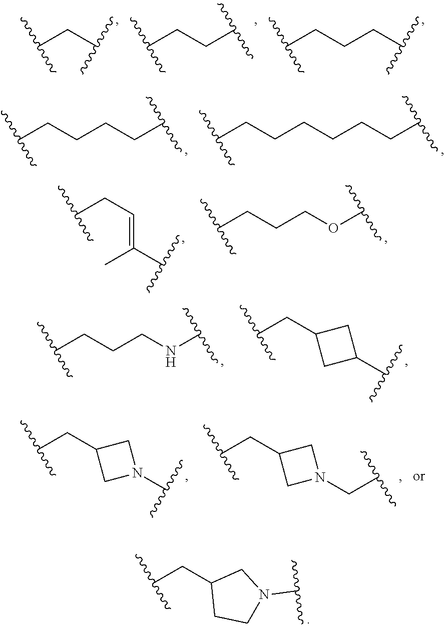

Embodiment P32. The compound of one of embodiments P1 to P31, wherein R⁵ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment P33. The compound of one of embodiments P1 to P31, wherein R⁵ is hydrogen, halogen, —CF₃, —CHF₂, —CH₂F, —OCF₃, —OCHF₂, —OCH₂F, —CN, —SF₅, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl.

Embodiment P34. The compound of one of embodiments P1 to P31, wherein R⁵ is hydrogen, —CF₃, —CHF₂, —OCF₃,

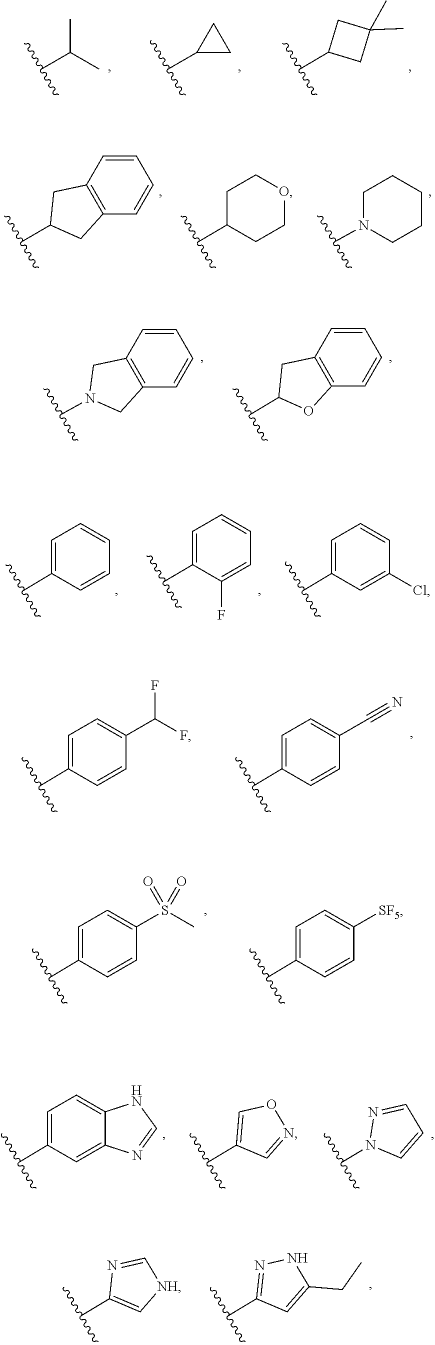

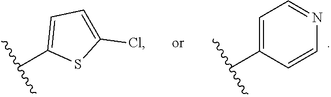 [SMILES: *c1ccc(Cl)s1 ; *c1ccncc1]

Embodiment P35. The compound of one of embodiments P1 to P27, wherein -L²-R⁵ is

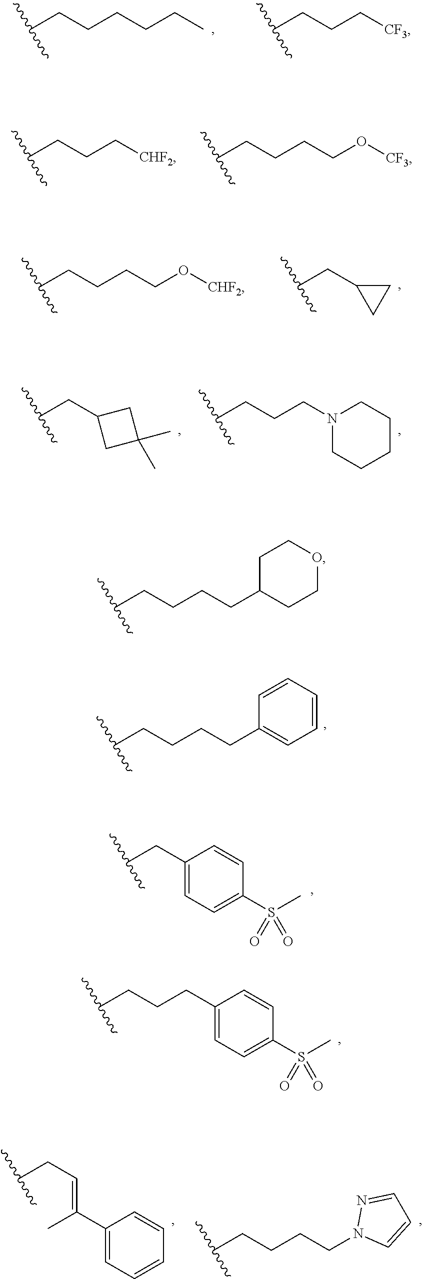

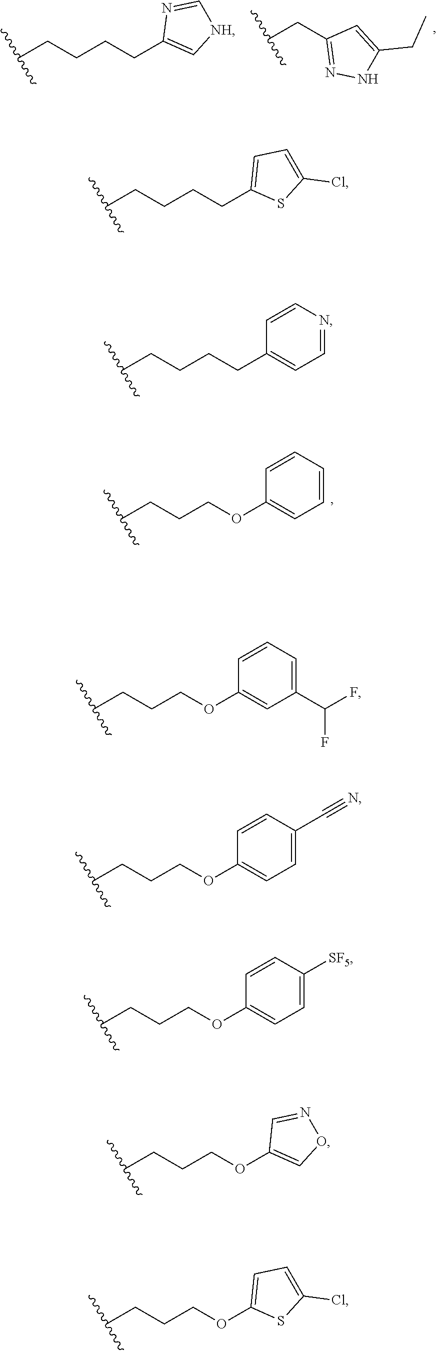

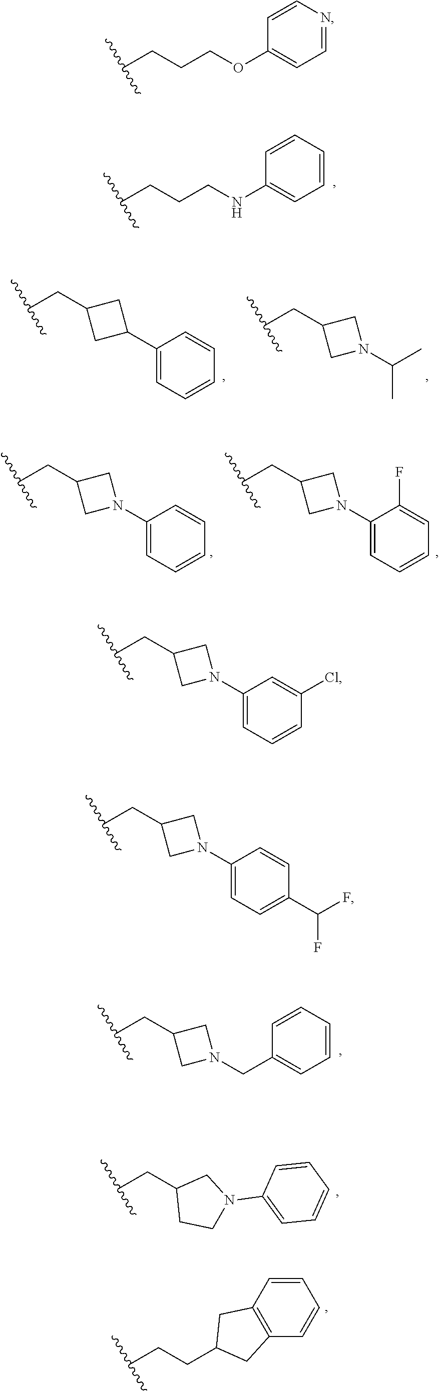

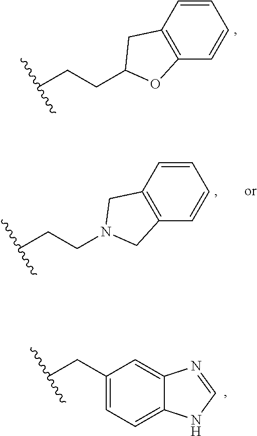 [SMILES: *CCC1Cc2ccccc2O1 ; *CCN1Cc2ccccc2C1 ; *Cc1ccc2[nH]cnc2c1]

Embodiment P36. A pharmaceutical composition comprising the compound of one of embodiments P1 to P35, or a pharmaceutically acceptable salt thereof, and a pharmaceutically acceptable excipient.

Embodiment P37. A method of treating a fibrotic disease in a subject in need thereof, said method comprising administering to the subject in need thereof a therapeutically effective amount of an acid ceramidase inhibitor, or a pharmaceutically acceptable salt thereof.

Embodiment P38. A method of treating a fibrotic disease in a subject in need thereof, said method comprising administering to the subject in need thereof a therapeutically effective amount of a compound of one of embodiments P1 to P35, or a pharmaceutically acceptable salt thereof.

Embodiment P39. The method of embodiment P38, wherein the fibrotic disease is nonalcoholic steatohepatitis or liver fibrosis.

Embodiment P40. A method of treating cancer in a subject in need thereof, said method comprising administering to the subject in need thereof a therapeutically effective amount of a compound of one of embodiments P1 to P35, or a pharmaceutically acceptable salt thereof.

Embodiment P41. The method of embodiment P40, wherein the cancer is liver cancer.

### VI. Additional Embodiments

Embodiment 1. A compound, or a pharmaceutically acceptable salt thereof, having the formula:

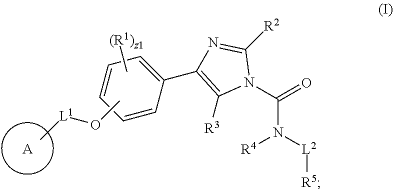

wherein
L¹ is a bond, —C(O)—, substituted or unsubstituted alkylene, or substituted or unsubstituted heteroalkylene;
Ring A is substituted or unsubstituted cycloalkyl, substituted or unsubstituted heterocycloalkyl, substituted or unsubstituted aryl, or substituted or unsubstituted heteroaryl;
R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)OR^{1C}, —C(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R¹ substituents may optionally be joined to form a substituted or unsubstituted cycloalkyl, substituted or unsubstituted heterocycloalkyl, substituted or unsubstituted aryl, or substituted or unsubstituted heteroaryl;
z1 is an integer from 0 to 4;
R² is hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ON^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —N^{2A}SO₂R^{2D}, —NR²AC(O)R^{2C}, —NR²AC(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is hydrogen, halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NR^{3C}NR^{3A}R^{3B}, —ONR^{3A}R^{3B}, —NHC(O)NR^{3C}NR^{3A}R^{3B}, —NHC(O)NR^{3A}R^{3B}, —N(O)ₘ₃, —NR^{3A}R^{3B}, —C(O)R^{3C}, —C(O)OR^{3C}, —C(O)NR^{3A}R^{3B}, —OR^{3D}, —SR^{3D}, —NR^{3A}SO₂R^{3D}, —NR³AC(O)R^{3C}, —NR³AC(O)OR^{3C}, —NR^{3A}OR^{3C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁴ is hydrogen or unsubstituted C₁-C₄ alkyl;
L² is -L^{2A}-L^{2B}-L^{2C}.
L^{2A}, L^{2B}, and L^{2C} are independently a bond, —O—, —NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R⁵ is hydrogen, halogen, —CX⁵₃, —CHX⁵₂, —CH₂X⁵, —OCX⁵₃, —OCH₂X⁵, —OCHX⁵₂, —CN, —SOₙ₅R^{5D}, —SOᵥ₅NR^{5A}R^{5B}, —NR^{5C}NR^{5A}R^{5B}, —ONR^{5A}R^{5B}, —NHC(O)NR^{5C}NR^{5A}R^{5B}, —NHC(O)NR^{5A}R^{5B}, —N(O)ₘ₅, —NR^{5A}R^{5B}, —C(O)R^{5C}, —C(O)OR^{5C}, —C(O)NR^{5A}R^{5B}, —OR^{5D}, —SR^{5D}, —NR^{5A}SO₂R^{5D}, —NR^{5A}C(O)R^{5C}, —NR^{5A}C(O)OR^{5C}, —NR^{5A}OR^{5C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{1A}, R^{1B}, R^{1C}, R^{1D}, R^{2A}, R^{2B}, R^{2C}, R^{2D}, R^{3A}, R^{3B}, R^{3C}, R^{3D}, R^{5A}, R^{5B}, R^{5C}, and R^{5D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{5A} and R^{5B} substituents bonded to the same nitrogen atom may optionally be joined to form a substituted or unsubstituted heterocycloalkyl or substituted or unsubstituted heteroaryl;
X¹, X², X³, and X⁵ are independently —F, —Cl, —Br, or —I;
n1, n2, n3, and n5 are independently an integer from 0 to 4; and
m1, m2, m3, m5, v1, v2, v3, and v5 are independently 1 or 2.

Embodiment 2. The compound of embodiment 1, having the formula:

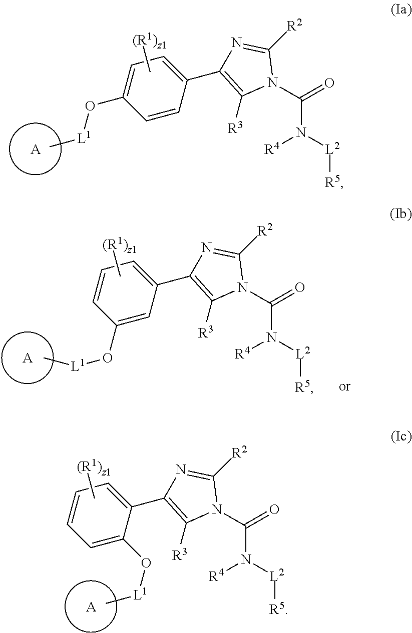

Embodiment 3. The compound of embodiment 1, having the formula:

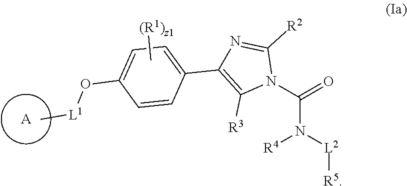

Embodiment 4. The compound of one of embodiments 1 to 3, wherein Ring A is substituted or unsubstituted 5 to 9 membered cycloalkyl, substituted or unsubstituted 5 to 6 membered heterocycloalkyl, substituted or unsubstituted phenyl, or substituted or unsubstituted 5 to 9 membered heteroaryl.

Embodiment 5. The compound of embodiment 1, having the formula:

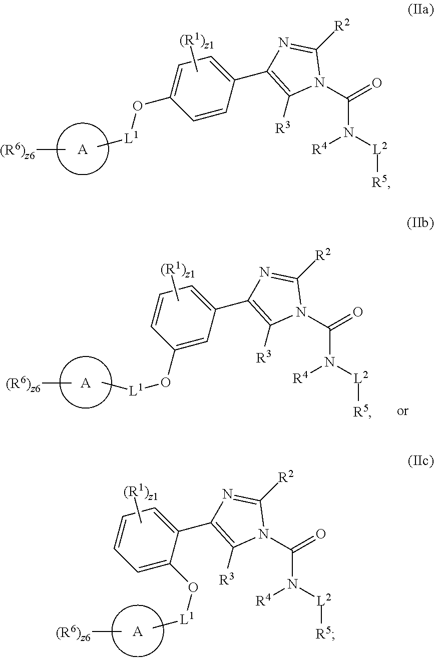

wherein
Ring A is cycloalkyl, heterocycloalkyl, aryl, or heteroaryl;
R⁶ is independently oxo, halogen, —CX⁶₃, —CHX⁶₂, —CH₂X⁶, —OCX⁶₃, —OCH₂X⁶, —OCHX⁶₂, —CN, —SOₙ₆R^{5D}, —S(O)(NH)R^{6D}, —SOᵥ₆NR^{6A}R^{6B}, —ONR^{6A}R^{6B}, —NHC(O)NR⁶CNR^{6A}R^{6B}, —NHC(O)NR^{6A}R^{6B}, —N(O)ₘ₆, —NR^{6A}R^{6B}, —C(O)R^{6C}, —C(O)OR^{6C}, —C(O)NR^{6A}R^{6B}, —OR^{6D}, —SR^{6D}, —NR^{6A}SO₂R^{6D}, —NR^{6A}C(O)R^{6C}, —NR^{6A}C(O)OR^{6C}, —NR^{6A}OR^{6C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R⁶ substituents may optionally be joined to form a substituted or unsubstituted cycloalkyl, substituted or unsubstituted heterocycloalkyl, substituted or unsubstituted aryl, or substituted or unsubstituted heteroaryl;
R^{6A}, R^{6B}, R^{6C}, and R^{6D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{6A} and R^{6B} substituents bonded to the same nitrogen atom may optionally be joined to form a substituted or unsubstituted heterocycloalkyl or substituted or unsubstituted heteroaryl;
X⁶ is independently —F, —Cl, —Br, or —I;
n6 is independently an integer from 0 to 4;
m6 and v6 are independently 1 or 2; and
z6 is an integer from 0 to 11.

Embodiment 6. The compound of embodiment 5, having the formula:

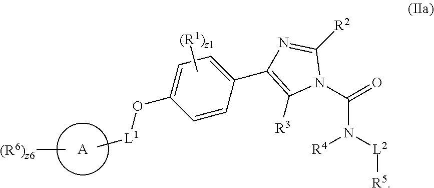

Embodiment 7. The compound of one of embodiments 5 to 6, wherein

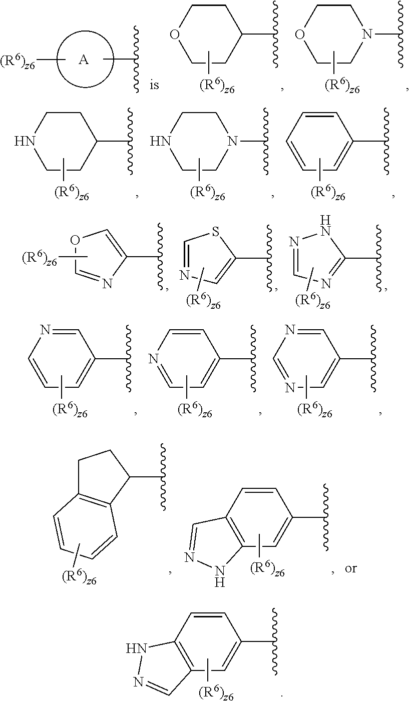

Embodiment 8. The compound of one of embodiments 5 to 7, wherein R⁶ is independently halogen, —CF₃, —CN, —SOₙ₆R^{6D}, —S(O)(NH)R^{6D}, —SOᵥ₆NR^{6A}R^{6B}, —NR^{6A}R^{6B}, —C(O)NR^{6A}R^{6B}, —OR^{6D}, —SR^{6D}, —NR^{6A}SO₂R^{6D}, —NR^{6A}C(O)R^{6C}, substituted or unsubstituted C₁-C₆ alkyl, substituted or unsubstituted 2 to 6 membered heteroalkyl, or substituted or unsubstituted 3 to 6 membered heterocycloalkyl.

Embodiment 9. The compound of one of embodiments 5 to 8, wherein R^{6A} and R^{6B} are independently hydrogen, substituted or unsubstituted C₁-C₄ alkyl, substituted or unsubstituted 2 to 6 membered heteroalkyl, or substituted or unsubstituted 3 to 6 membered heterocycloalkyl.

Embodiment 10. The compound of one of embodiments 5 to 8, wherein R^{6A} and R^{6B} substituents bonded to the same nitrogen atom are joined to form a substituted or unsubstituted 3 to 6 membered heterocycloalkyl.

Embodiment 11. The compound of one of embodiments 5 to 8, wherein R^{6C} is independently unsubstituted C₃-C₆ cycloalkyl or substituted or unsubstituted 3 to 6 membered heterocycloalkyl.

Embodiment 12. The compound of one of embodiments 5 to 8, wherein R^{6D} is independently hydrogen, —CHF₂, substituted or unsubstituted C₁-C₄ alkyl, unsubstituted 2 to 6 membered heteroalkyl, or substituted or unsubstituted 3 to 6 membered heterocycloalkyl.

Embodiment 13. The compound of one of embodiments 5 to 7, wherein R⁶ is independently —F, —CF₃, —CN, —SO₂CH₃, —SO₂NH₂, —SO₂NHCH₃, —S(O)CH₃, —S(O)(NH)CH₃,

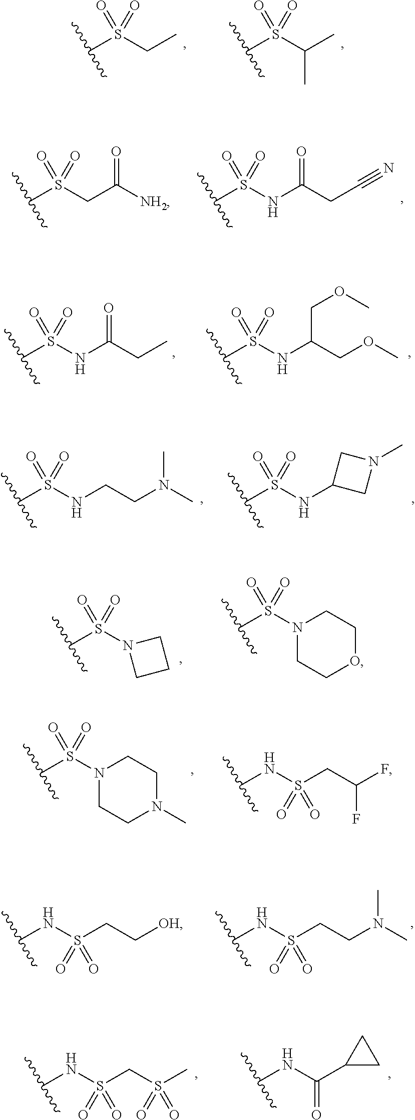

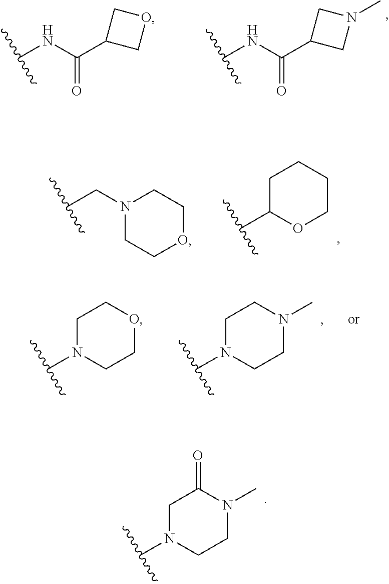

Embodiment 14. The compound of one of embodiments 5 to 7, wherein z6 is 0.

Embodiment 15. The compound of one of embodiments 5 to 13, wherein z6 is 1.

Embodiment 16. The compound of one of embodiments 5 to 13, wherein z6 is 2.

Embodiment 17. The compound of one of embodiments 5 to 7, wherein

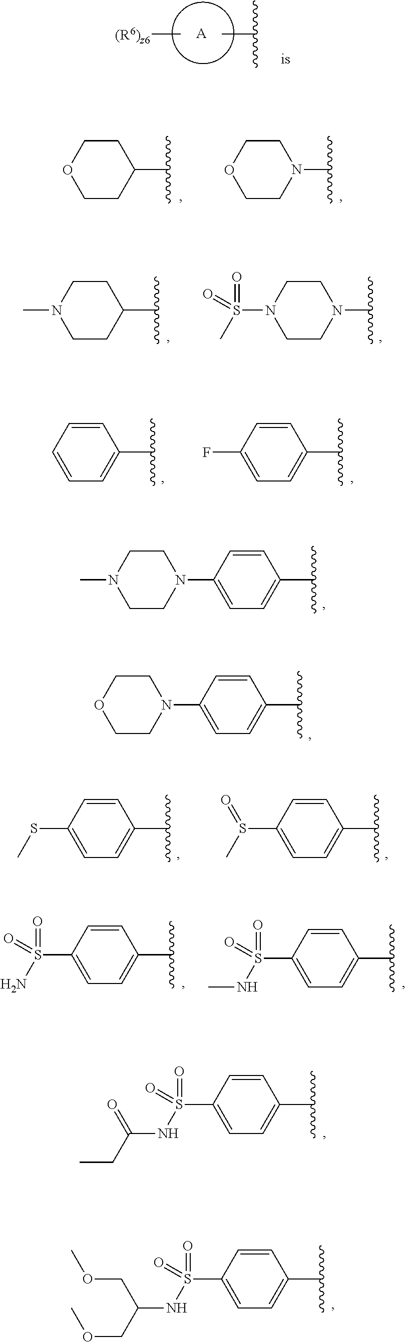

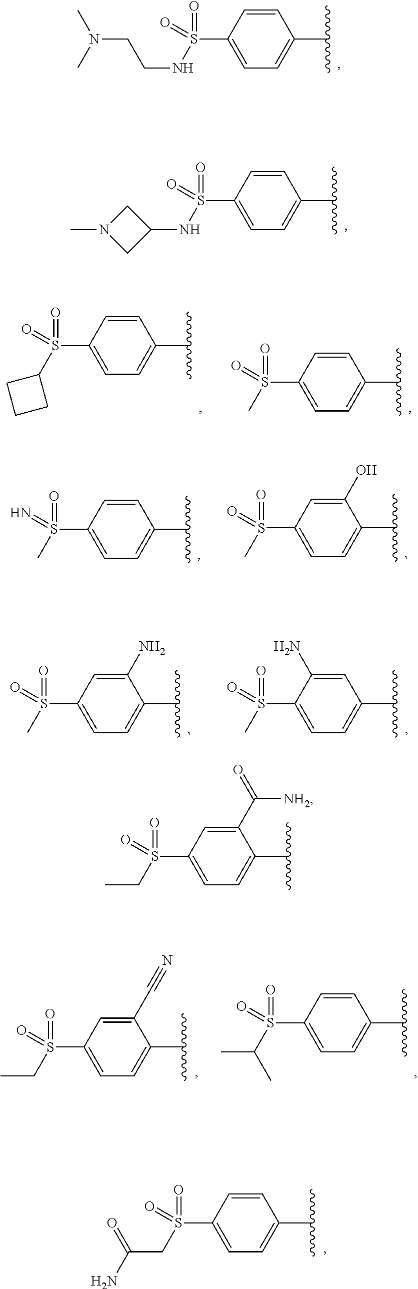

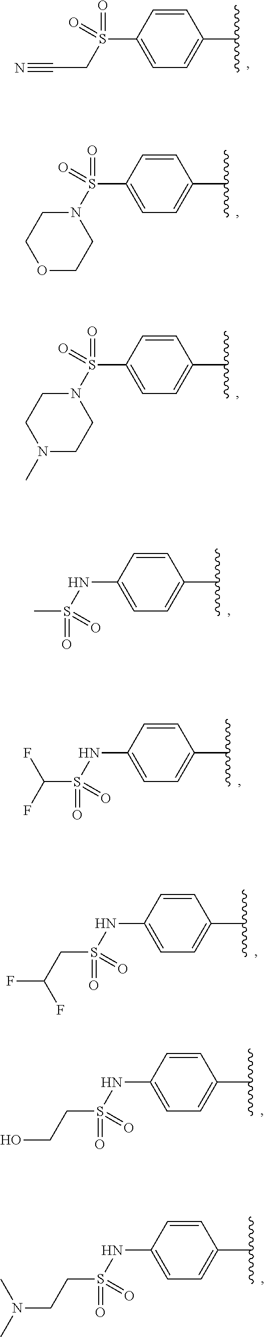

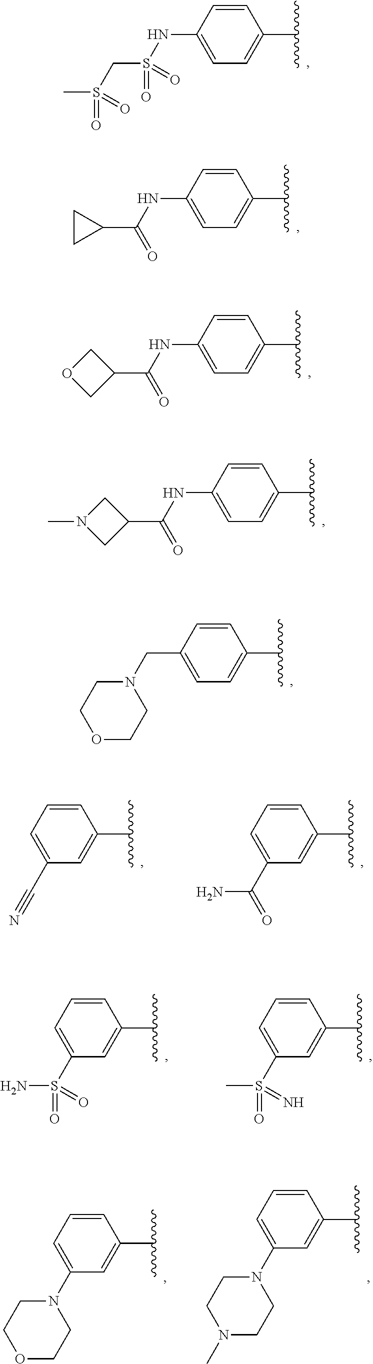

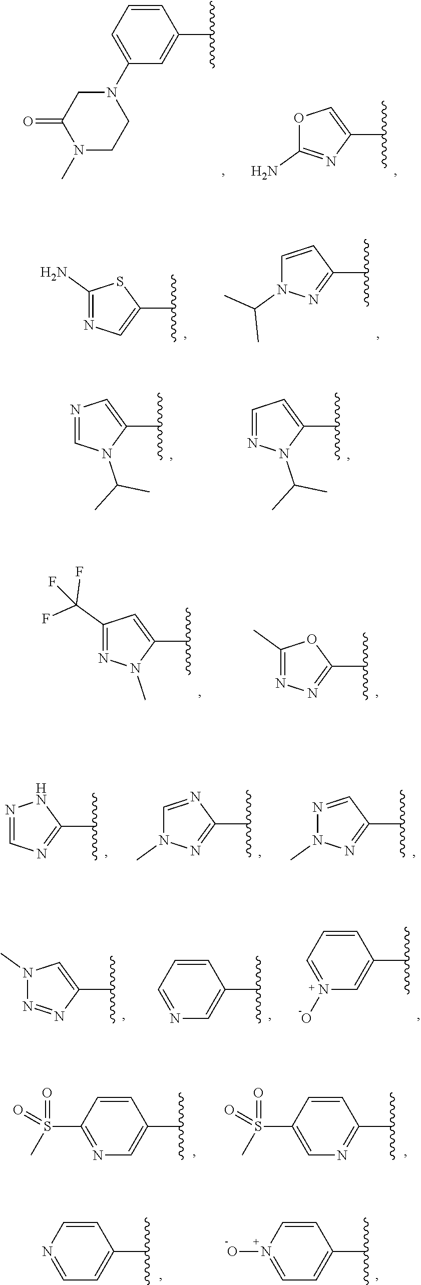

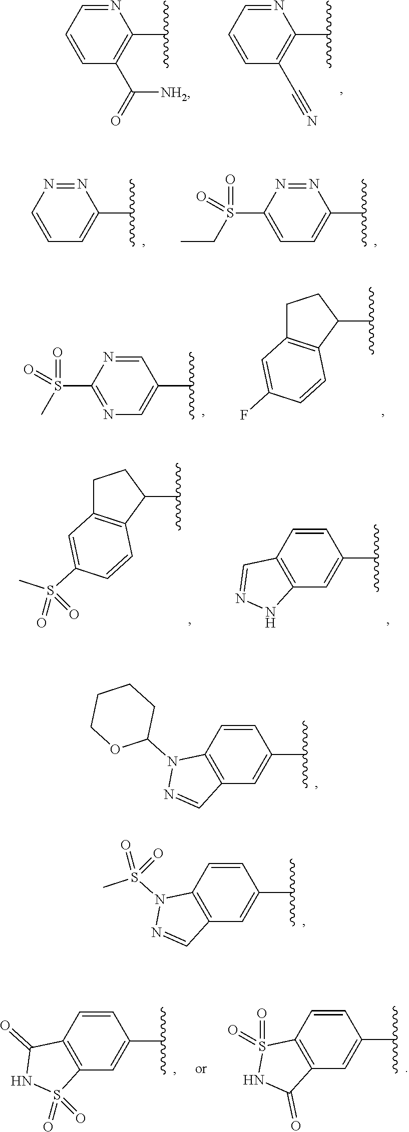

Embodiment 18. The compound of one of embodiments 1 to 17, wherein L¹ is a bond, —C(O)—, substituted or unsubstituted C₁-C₄ alkylene, or substituted or unsubstituted 2 to 4 membered heteroalkylene.

Embodiment 19. The compound of one of embodiments 1 to 17, wherein L¹ is a bond, —C(O)—,

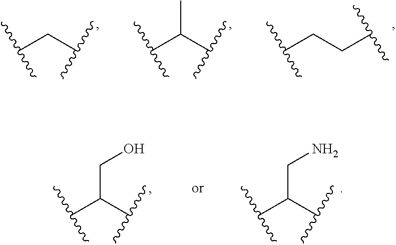

Embodiment 20. The compound of one of embodiments 1 to 19, wherein R¹ is independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment 21. The compound of one of embodiments 1 to 19, wherein z1 is 0.

Embodiment 22. The compound of one of embodiments 1 to 21, wherein R² is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment 23. The compound of one of embodiments 1 to 21, wherein R² is hydrogen or halogen.

Embodiment 24. The compound of one of embodiments 1 to 23, wherein R³ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment 25. The compound of one of embodiments 1 to 23, wherein R³ is hydrogen or halogen.

Embodiment 26. The compound of one of embodiments 1 to 25, wherein R⁴ is hydrogen or unsubstituted methyl.

Embodiment 27. The compound of one of embodiments 1 to 25, wherein R⁴ is hydrogen.

Embodiment 28. The compound of one of embodiments 1 to 27, wherein L^{2A} is unsubstituted C₁-C₆ alkylene.

Embodiment 29. The compound of one of embodiments 1 to 28, wherein L^{2B} is a bond, —O—, —NH—, unsubstituted C₁-C₆ alkylene, unsubstituted C₃-C₆ cycloalkylene, or unsubstituted 3 to 6 membered heterocycloalkylene.

Embodiment 30. The compound of one of embodiments 1 to 29, wherein L^{2C} is a bond, —O—, —NH—, unsubstituted C₁-C₆ alkylene, unsubstituted phenylene, or unsubstituted 5 to 6 membered heteroarylene.

Embodiment 31. The compound of one of embodiments 1 to 27, wherein L² is

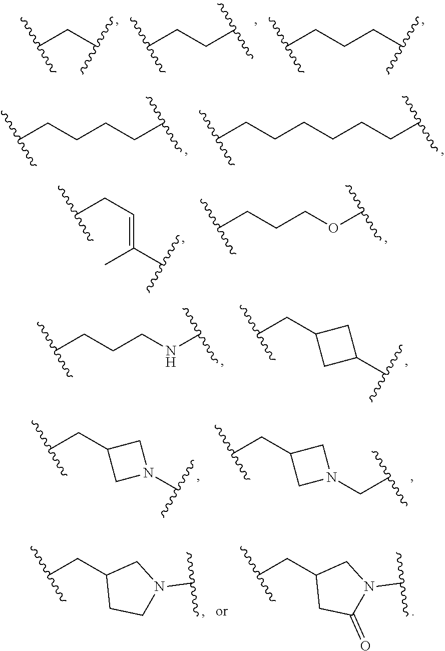

Embodiment 32. The compound of one of embodiments 1 to 31, wherein R⁵ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment 33. The compound of one of embodiments 1 to 31, wherein R⁵ is hydrogen, halogen, —CF₃, —CHF₂, —CH₂F, —OCF₃, —OCHF₂, —OCH₂F, —CN, —SF₅, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl.

Embodiment 34. The compound of one of embodiments 1 to 31, wherein R⁵ is hydrogen, —CF₃, —CHF₂, —OCF₃

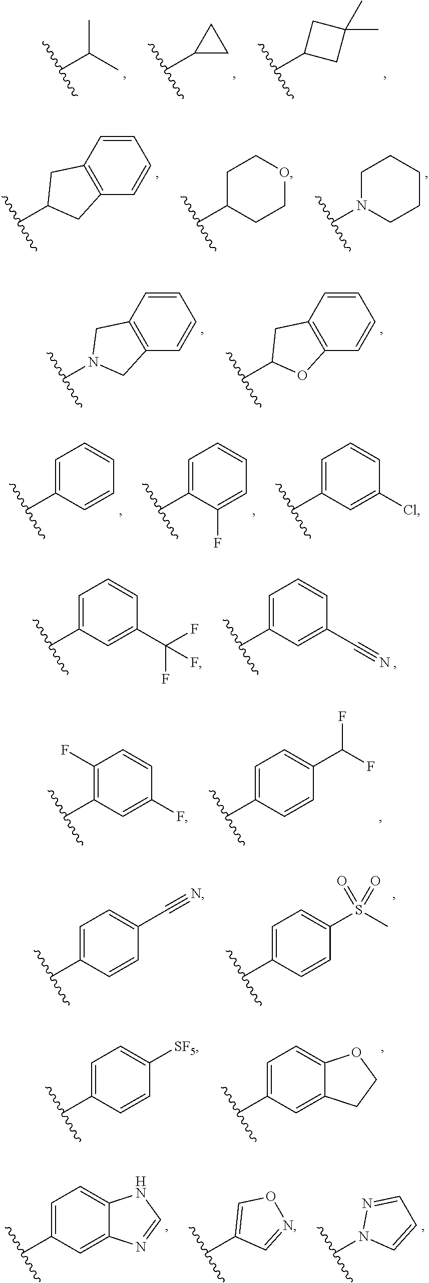

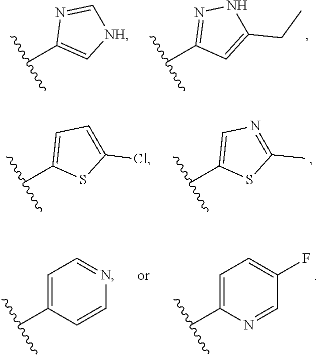

Embodiment 35. The compound of one of embodiments 1 to 27, wherein -L²-R⁵ is

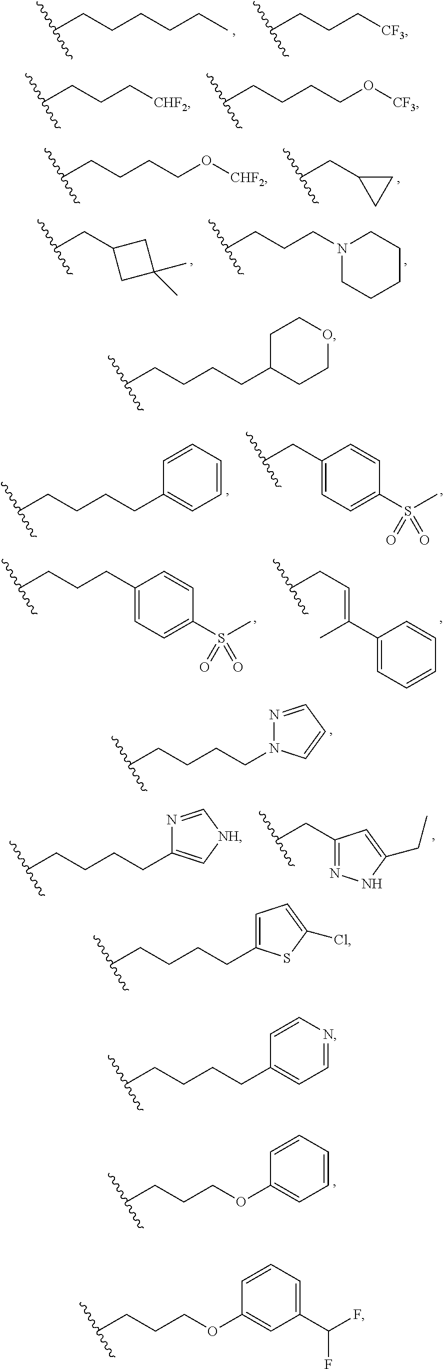

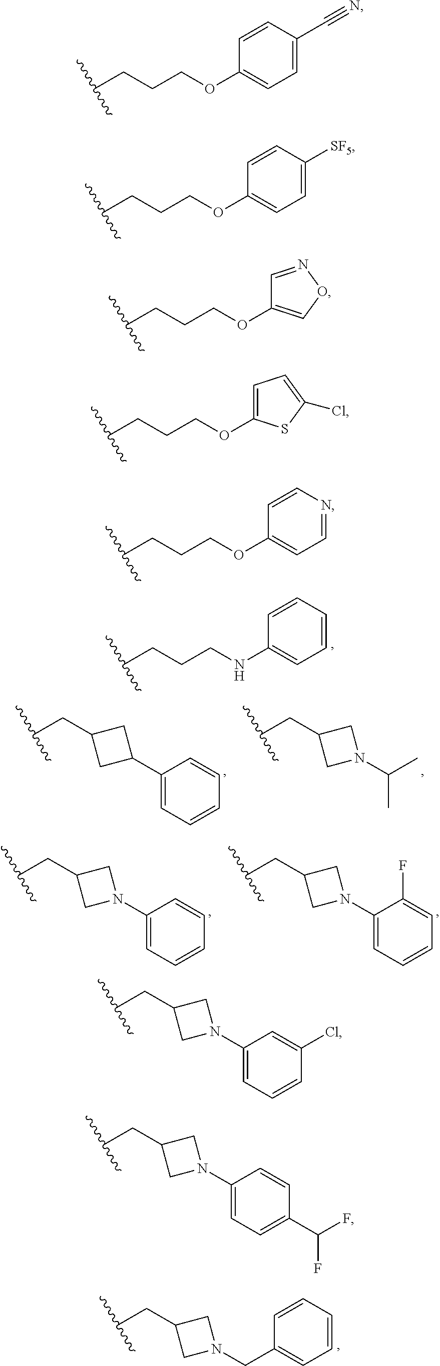

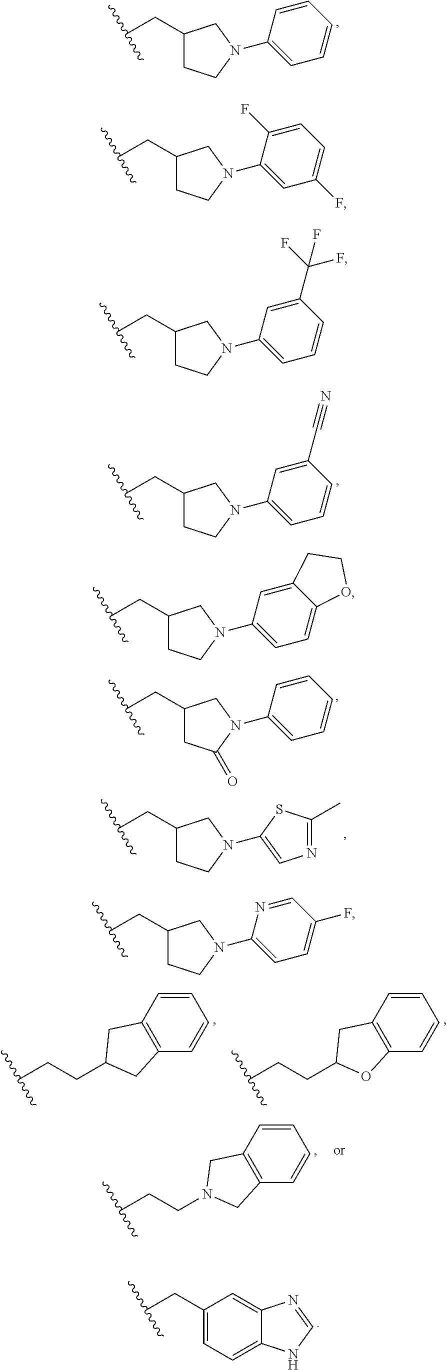

Embodiment 36. A pharmaceutical composition comprising the compound of one of embodiments 1 to 35, or a pharmaceutically acceptable salt thereof, and a pharmaceutically acceptable excipient.

Embodiment 37. A method of treating a fibrotic disease in a subject in need thereof, said method comprising administering to the subject in need thereof a therapeutically effective amount of an acid ceramidase inhibitor, or a pharmaceutically acceptable salt thereof.

Embodiment 38. A method of treating a fibrotic disease in a subject in need thereof, said method comprising administering to the subject in need thereof a therapeutically effective amount of a compound of one of embodiments 1 to 35, or a pharmaceutically acceptable salt thereof.

Embodiment 39. The method of embodiment 38, wherein the fibrotic disease is nonalcoholic steatohepatitis or liver fibrosis.

Embodiment 40. A method of treating cancer in a subject in need thereof, said method comprising administering to the subject in need thereof a therapeutically effective amount of a compound of one of embodiments 1 to 35, or a pharmaceutically acceptable salt thereof.

Embodiment 41. The method of embodiment 40, wherein the cancer is liver cancer.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### Examples

### Example 1: Experimental Procedures and Characterization Data

General information: All evaporations were carried out in vacuo with a rotary evaporator. Analytical samples were dried in vacuo (1-5 mmHg) at rt. Thin layer chromatography (TLC) was performed on silica gel plates, spots were visualized by UV light (214 and 254 nm). Purification by column and flash chromatography was carried out using silica gel (200-300 mesh). Solvent systems are reported as mixtures by volume. All NMR spectra were recorded on a Bruker 400 (400 MHz) spectrometer. ¹H chemical shifts are reported in 8 values in ppm with the deuterated solvent as the internal standard. Data are reported as follows: chemical shift, multiplicity (s=singlet, d=doublet, t=triplet, q=quartet, br=broad, m=multiplet), coupling constant (Hz), integration. LCMS spectra were obtained on an Agilent 1200 series 6110 or 6120 mass spectrometer with electrospray ionization and excepted as otherwise indicated, the general LCMS condition was as follows: Waters X Bridge C18 column (50 mm×4.6 mm×3.5 um), Flow Rate: 2.0 m1/min, the column temperature: 40° C.

AC2020601-0167

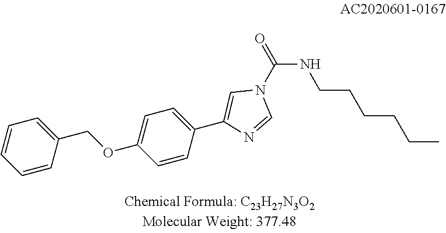 [SMILES: CCCCCCNC(=O)n1cnc(-c2ccc(OCc3ccccc3)cc2)c1]

### The Synthesis of 1-(4-(benzyloxy)phenyl)ethanone (0149-2)

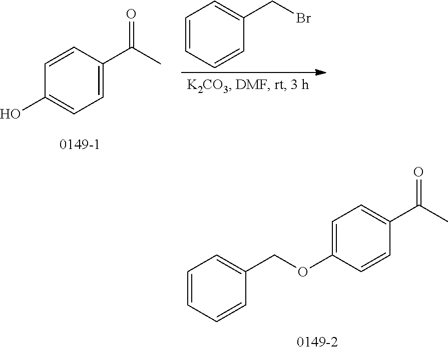

To a stirred solution of 0149-1 (680 mg, 5.0 mmol) in DMF (20 mL) was added K₂CO₃ (1.38 g, 10.0 mmol) and (bromomethyl)benzene (0.6 mL, 5.0 mmol), and the reaction mixture was allowed to stir at room temperature for 3 hours. After the reaction finished (by LCMS), water (50 mL) was added, extracted with EtOAc, the combined organic layer was washed with brine, dried over anhydrous Na₂SO₄, filtered and evaporated under reduced pressure. The crude was recrystallized with CH₂Cl₂ and hexane to afford 0149-2 (1.09 g, yield: 96.5%) as a white solid. LC-MS t_{R}=1.875 min.

### The Synthesis of 1-(4-(benzyloxy)phenyl)-2-bromoethanone (0149-3)

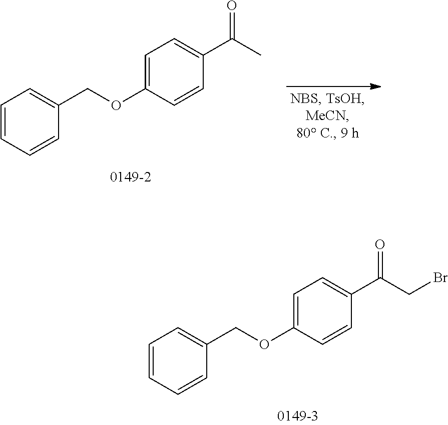 [SMILES: CC(=O)c1ccc(OCc2ccccc2)cc1 ; O=C(CBr)c1ccc(OCc2ccccc2)cc1]

To a stirred solution of 0149-2 (1.09 g, 4.8 mmol) in MeCN (30 mL) was added TsOH (90 mg, 0.5 mmol) and NBS (855 mg, 4.8 mmol), and this mixture was heated to 80° C. for 9 hours. After the reaction was completed (by LCMS), the mixture was concentrated under reduced pressure and dissolved in EtOAc (50 mL). The residue was washed with water and brine, dried over anhydrous Na₂SO₄, filtered and evaporated under reduced pressure. The crude was purified by prep-HPLC to give 0149-3 (780 mg, yield: 53.4%) as a white solid. LC-MS t_{R}=1.964 min.

### The Synthesis of 4-(4-(benzyloxy)phenyl)-1H-imidazole (0149-4)

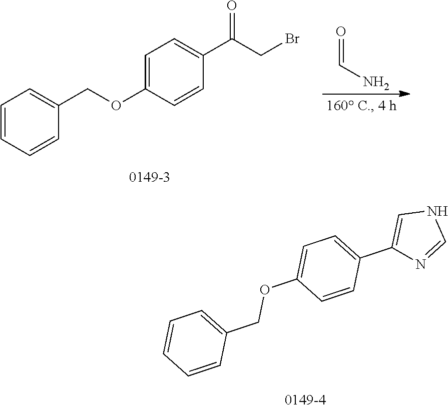

A stirred solution of PJ2-0149-3 (400 mg, 1.31 mmol) in formamide (5 mL) was heated to 160° C. for 4 hours. Then the reaction mixture was poured into water (20 mL), extracted with EtOAc, the combined organic layer was washed with water and brine, dried over anhydrous Na₂SO₄, filtered and evaporated under reduced pressure. The crude was purified by silica gel chromatography (5-20% MeOH in CH₂Cl₂) to get 0149-4 (310 mg, yield: 94.7%) as a pink solid.

### The Synthesis of 4-(4-(benzyloxy)phenyl)-N-hexyl-1H-imidazole-1-carboxamide (AC2020601-0149)

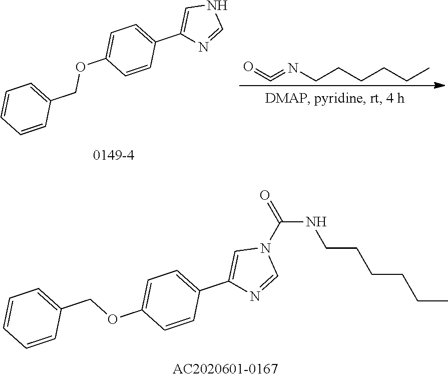

To a stirred solution of 0149-4 (150 mg, 0.60 mmol) in pyridine (5 mL) was added DMAP (7 mg, 0.06 mmol) and 1-isocyanatohexane (69 mg, 0.54 mmol), and this mixture was allowed to stir at room temperature for 4 hours. After the reaction was completed (by LCMS), the mixture was concentrated under reduced pressure and dissolved in EtOAc (20 mL). The residue was washed with water and brine, dried over anhydrous Na₂SO₄, filtered and evaporated under reduced pressure. The crude was purified by silica gel chromatography (20-33% EtOAc in hexane) and recrystallized with EtOAc and hexane to give AC2020601-0167 (17 mg, yield: 12.8%) as a white solid. ¹H NMR (400 MHz, CDCl₃) δ 8.27 (s, 1H), 7.71 (d, J=8.8 Hz, 2H), 7.41-7.47 (m, 3H), 7.38 (t, J=8.8 Hz, 2H), 7.31-7.33 (m, 1H), 7.00-7.03 (m, 2H), 5.78 (t, J=1.2 Hz, 1H), 5.10 (s, 2H), 3.43-3.49 (m, 2H), 1.62-1.70 (m, 2H), 1.31-1.42 (m, 6H), 0.91 (t, J=6.8 Hz, 3H). LC-MS m/z: 378.1 [M+H]⁺. HPLC Purity (254 nm): 95.81%; t_{R}=9.348 min.

AC2020601-0199

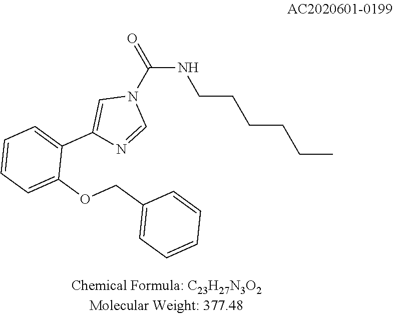 [SMILES: CCCCCCNC(=O)n1cnc(-c2ccccc2OCc2ccccc2)c1]

### The Synthesis of 1-(2-(benzyloxy)phenyl)ethanone (0123-2)

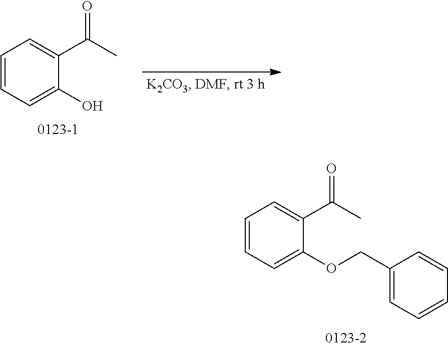

To a stirred solution of 0123-1 (1.02 g, 7.5 mmol) in DMF (50 mL) was added K₂CO₃ (2.07 g, 15.0 mmol) and (bromomethyl)benzene (1.5 mL, 7.9 mmol), and the reaction mixture was allowed to stir at room temperature for 3 hours. After the reaction finished (by LCMS), water (50 mL) was added, extracted with EtOAc, the combined organic layer was washed with brine, dried over anhydrous Na₂SO₄, filtered and evaporated under reduced pressure. The crude was recrystallized with CH₂Cl₂ and hexane to afford 0123-2 (1.71 g, yield: 99.8%) as yellow oil.

### The Synthesis of 1-(2-(benzyloxy)phenyl)-2-bromoethanone (0123-3)

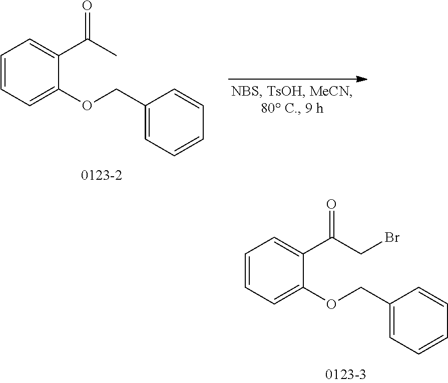 [SMILES: CC(=O)c1ccccc1OCc1ccccc1 ; O=C(CBr)c1ccccc1OCc1ccccc1]

To a stirred solution of 0123-2 (1.71 g, 7.5 mmol) in MeCN (30 mL) was added TsOH (129 mg, 0.75 mmol) and NBS (1.60 g, 9.0 mmol), and this mixture was heated to 80° C. for 9 hours. After the reaction was completed (by LCMS), the mixture was concentrated under reduced pressure and dissolved in EtOAc (50 mL). The residue was washed with water and brine, dried over anhydrous Na₂SO₄, filtered and evaporated under reduced pressure. The crude was purified by prep-HPLC to give 0123-3 (1.02 g, yield: 44.5%) as colorless oil. LC-MS t_{R}=1.989 min.

### The Synthesis of 4-(2-(benzyloxy)phenyl)-1H-imidazole (0123-4)

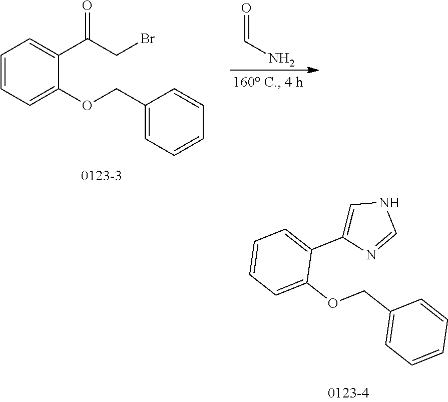

A stirred solution of PJ2-0123-3 (170 mg, 0.557 mmol) in formamide (2 mL) was heated to 160° C. for 4 hours. Then the reaction mixture was poured into water (100 mL), extracted with EtOAc, the combined organic layer was washed with water and brine, dried over anhydrous Na₂SO₄, filtered and evaporated under reduced pressure. The crude was purified by silica gel chromatography (50-100% EtOAc in hexane) to get 0123-4 (120 mg, yield: 85.8%) as a white solid.

### The Synthesis of 4-(2-(benzyloxy)phenyl)-N-hexyl-1H-imidazole-1-carboxamide (AC2020601-0199)

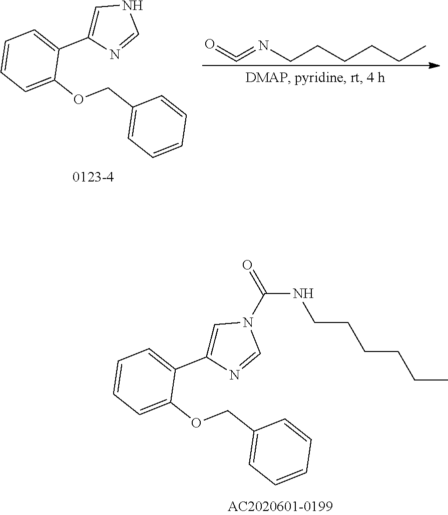

To a stirred solution of 0123-4 (120 mg, 0.48 mmol) in pyridine (5 mL) was added DMAP (7 mg, 0.06 mmol) and 1-isocyanatohexane (57 mg, 0.45 mmol), and this mixture was allowed to stir at room temperature for 4 hours. After the reaction was completed (by LCMS), the mixture was concentrated under reduced pressure and dissolved in EtOAc (20 mL). The residue was washed with water and brine, dried over anhydrous Na₂SO₄, filtered and evaporated under reduced pressure. The crude was purified by silica gel chromatography (20-50% EtOAc in hexane) get AC2020601-0199 (165 mg, yield: 90.9%) as a white solid. ¹H NMR (400 MHz, CDCl₃) δ 8.25 (dd, J=7.6 Hz, J=1.6 Hz, 1H), 8.22 (d, J=1.2 Hz, 1H), 7.63 (d, J=1.2 Hz, 1H), 7.52-7.54 (m, 2H), 7.39-7.46 (m, 3H), 7.26-7.30 (m, 1H), 7.08-7.12 (m, 1H), 7.05 (d, J=8.0 Hz, 1H), 5.19 (s, 2H), 5.13 (br, 1H), 3.31-3.36 (m, 2H), 1.15-1.55 (m, 2H), 1.32-1.44 (m, 6H), 0.92 (t, J=6.8 Hz, 3H). LC-MS m/z: 378.1 [M+H]⁺. HPLC Purity (254 nm): 96.98%; t_{R}=9.314 min.

AC2020601-0205

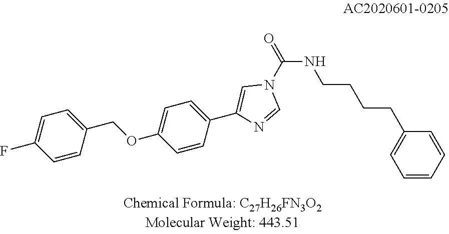 [SMILES: O=C(NCCCCc1ccccc1)n1cnc(-c2ccc(OCc3ccc(F)cc3)cc2)c1]

### The Synthesis of 1-(4-(4-fluorobenzyloxy)phenyl)ethanone (0205-2)

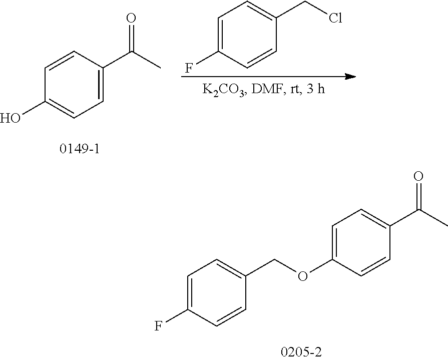

To a stirred solution of 0149-1 (680 mg, 5.0 mmol) in DMF (20 mL) was added K₂CO₃ (1.38 g, 10.0 mmol) and 1-(chloromethyl)-4-fluorobenzene (722 mg, 5.0 mmol). The reaction mixture was allowed to stir at room temperature for 3 hours. After the reaction finished (by LCMS), water (50 mL) was added, extracted with EtOAc (50 mL), the combined organic layer was washed with brine, dried over anhydrous Na₂SO₄, filtered and evaporated under reduced pressure. The crude was recrystallized with CH₂Cl₂/hexane to afford 0205-2 (1.09 g, yield: 89%) as a white solid.

### The Synthesis of 2-bromo-1-(4-(4-fluorobenzyloxy)phenyl)ethanone (0205

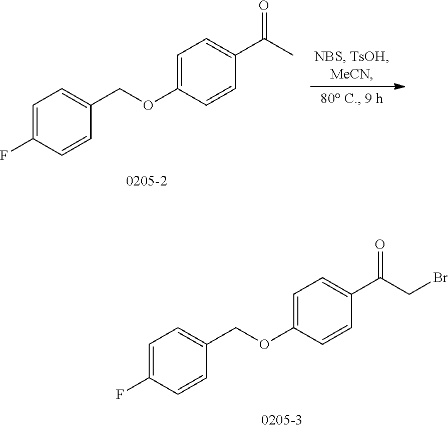 [SMILES: CC(=O)c1ccc(OCc2ccc(F)cc2)cc1 ; O=C(CBr)c1ccc(OCc2ccc(F)cc2)cc1]

To a stirred solution of 0205-2 (1.09 g, 4.46 mmol) in MeCN (30 mL) was added TsOH (90 mg, 0.5 mmol) and NBS (785 mg, 4.46 mmol). The reaction mixture was heated to 80° C. for 9 hours. After the reaction was completed (by LCMS), the mixture was concentrated under reduced pressure and dissolved in EtOAc (50 mL), washed with water and brine, dried over anhydrous Na₂SO₄, filtered and evaporated under reduced pressure. The crude was purified by prep-HPLC to give 0205-3 (1.20 g, yield: 83%) as a white solid.

### The Synthesis of 4-(4-(4-fluorobenzyloxy)phenyl)-1H-imidazole (0205-4)

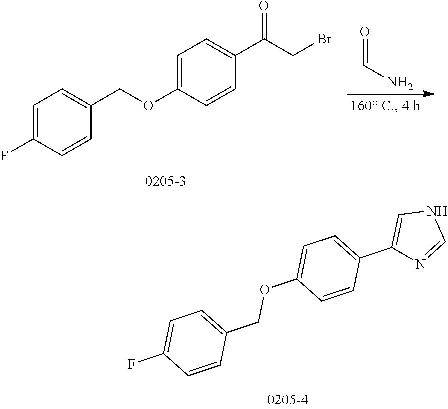

A stirred solution of 0205-3 (1.00 g, 3.09 mmol) in formamide (5 mL) was heated to 160° C. for 4 hours. Then the reaction mixture was poured into water (20 mL), extracted with EtOAc, the combined organic layer was washed with water and brine, dried over anhydrous Na₂SO₄, filtered and evaporated under reduced pressure. The crude was purified by silica gel chromatography (5-20% MeOH in CH₂Cl₂) to get 0205-4 (600 mg, yield: 72.3%) as a pink solid.

### The Synthesis of 4-(4-(4-fluorobenzyloxy)phenyl)-N-(4-phenylbutyl)-H-imidazole-1-a rboxamide (AC2020601-0205)

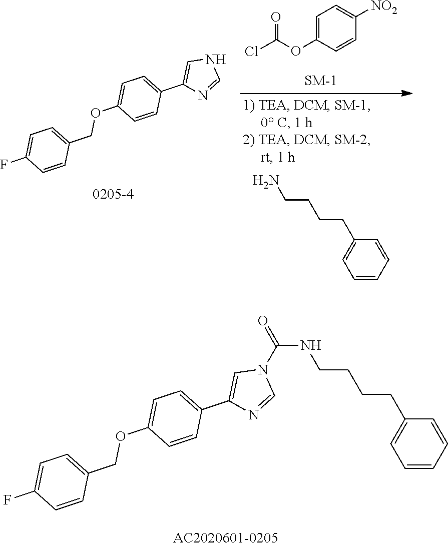 [SMILES: O=C(Cl)Oc1ccc([N+](=O)[O-])cc1 ; Fc1ccc(COc2ccc(-c3c[nH]cn3)cc2)cc1 ; NCCCCc1ccccc1 ; O=C(NCCCCc1ccccc1)n1cnc(-c2ccc(OCc3ccc(F)cc3)cc2)c1]

To a solution of 205-4 (268 mg, 1 mmol) in DCM(15 mL) was added SM-1(201 mg, 1 mmol) and TEA (202 mg, 1 mmol) at 0° C. The reaction mixture was stirred for 1 h at 0° C. then 4-phenylbutan-1-amine (149 mg, 1 mmol) was added. After 1 hour stirring at room temperature, the reaction mixture was quenched with water, extracted with EtOAc, the combined organic layer was washed with brine, dried over anhydrous Na₂SO₄ and evaporated under reduced pressure. The crude was purified by silica gel chromatography (50% EA/PE) to afford AC2020601-0205 (98 mg, yield: 22%) as a white solid. ¹H NMR (400 MHz, CDCl3) δ 8.12 (d, J=1.2 Hz, 1H), 7.70 (d, J=8.4 Hz, 2H), 7.39-7.45 (m, 3H), 7.27-7.29 (m, 2H), 7.17-7.20 (m, 3H), 7.07 (t, J=8.8 Hz, 2H), 6.99 (d, J=8.8 Hz, 2H), 5.74-5.84 (m, 1H), 5.04 (s, 2H), 3.43-3.74 (m, 2H), 2.67 (t, J=6.8 Hz, 2H), 1.59-1.76 (m, 4H). LC-MS m/z: 444.0 [M+H]⁺. HPLC Purity (254 nm): >99.9%; t_{R}=9.432 min.

AC2020601-0206

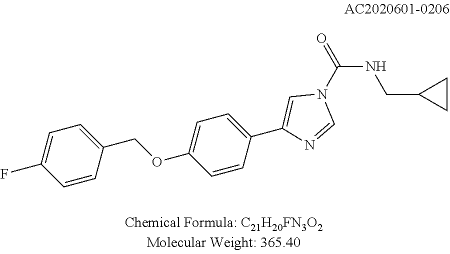 [SMILES: O=C(NCC1CC1)n1cnc(-c2ccc(OCc3ccc(F)cc3)cc2)c1]

### The Synthesis of N-(cyclopropylmethyl)-4-(4-(4-fluorobenzyloxy)Vphenyl)-1H-imid azole-1-carboxamide (AC2020601-0206)

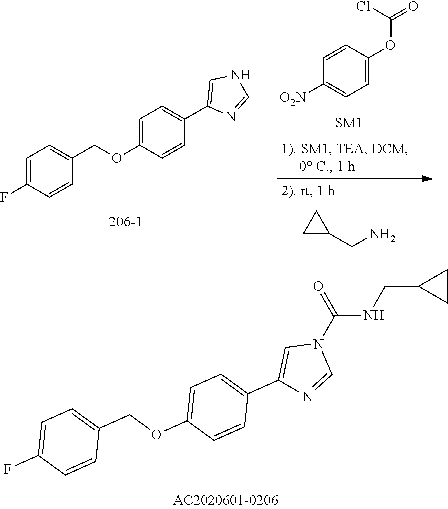 [SMILES: Fc1ccc(COc2ccc(-c3c[nH]cn3)cc2)cc1 ; O=C(Cl)Oc1ccc([N+](=O)[O-])cc1 ; NCC1CC1 ; O=C(NCC1CC1)n1cnc(-c2ccc(OCc3ccc(F)cc3)cc2)c1]

To a solution of 206-1 (268 mg, 1.0 mmol) in DCM (15 mL) was added SM-1 (201 mg, 1.0 mmol) and TEA (202 mg, 2.0 mmol) at 0° C. and stirred for 1 hour. Then the reaction mixture was allowed to warm to rt and cyclopropylmethanamine (149 mg, 1.0 mmol) was added. After 1 hour stirring, the reaction mixture was quenched with water, extracted with EtOAc, the combined organic layer was washed with brine, dried over anhydrous Na₂SO₄ and evaporated under reduced pressure. The crude was purified by silica gel chromatography (55% EA/PE) to afford AC2020601-0206 (28 mg, yield: 7%) as a white solid. ¹H NMR (400 MHz, CDCl₃) δ 8.18 (d, J=0.8 Hz, 1H), 7.73 (d, J=8.4 Hz, 2H), 7.49 (d, J=1.2 Hz, 1H), 7.42 (dd, J=5.6 Hz, 8.8 Hz, 2H), 7.08 (t, J=8.4 Hz, 2H), 6.99 (d, J=8.8 Hz, 2H), 5.74 (s, 1H), 5.05 (s, 2H), 3.32 (dd, J=5.6 Hz, 7.6 Hz, 2H), 1.07-1.11 (m, 1H), 0.61-0.65 (m, 2H), 0.32-0.34 (m, 2H). LC-MS m/z: 366.0 [M+H]⁺. HPLC Purity (254 nm): 98.37%; t_{R}=8.340 min.

AC2020601-0243

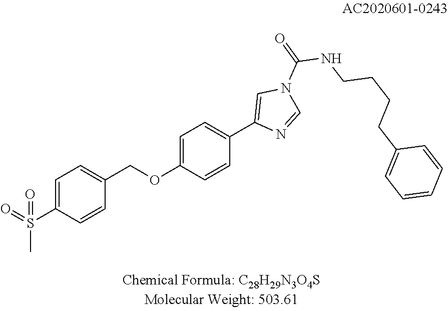 [SMILES: CS(=O)(=O)c1ccc(COc2ccc(-c3cn(C(=O)NCCCCc4ccccc4)cn3)cc2)cc1]

### The Synthesis of tert-butyl 4-(4-((4-(methylsulfonyl)benzyl)oxy)phenyl)-1H-imidazole-1-carboxylate (243-2)

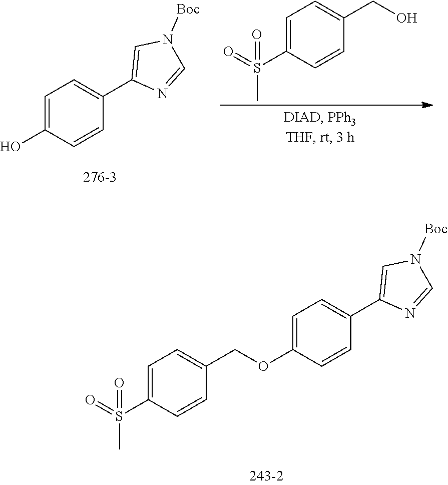 [SMILES: CC(C)(C)OC(=O)n1cnc(-c2ccc(O)cc2)c1 ; CS(=O)(=O)c1ccc(CO)cc1 ; CC(C)(C)OC(=O)n1cnc(-c2ccc(OCc3ccc(S(C)(=O)=O)cc3)cc2)c1]

To a suspension of 276-3 (780 mg, 3.0 mmol) in THF (10 mL) was added (4-(methylsulfonyl)phenyl)methanol (558 mg, 3.0 mmol), PPh₃ (942 mg, 3.6 mmol) and DIAD (789 mg, 3.9 mmol). The reaction mixture was stirred at room temperature for 3 hours until the reaction was complete (by LCMS). Saturated NaCl solution was added and the reaction mixture was extracted with EtOAc (20 mL), the combined organic layer was washed with brine, dried over anhydrous Na₂SO₄ and evaporated under reduced pressure. The crude was purified by silica gel chromatography (10% MeOH/DCM) to afford 243-2 (680 mg, yield: 53%) as yellow oil.

### The Synthesis of 4-(4-((4-(methylsulfonyl)benzyl)oxy)phenyl)-1H-imidazole (243-3)

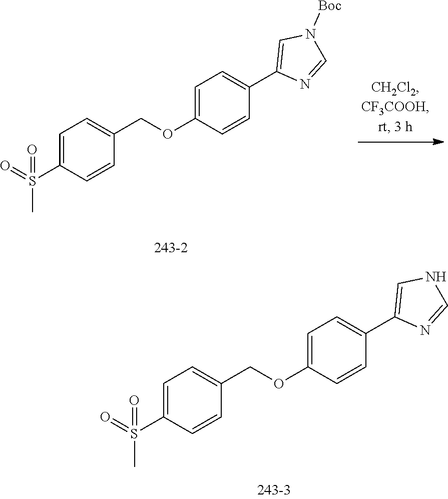 [SMILES: CC(C)(C)OC(=O)n1cnc(-c2ccc(OCc3ccc(S(C)(=O)=O)cc3)cc2)c1 ; CS(=O)(=O)c1ccc(COc2ccc(-c3c[nH]cn3)cc2)cc1]

To a stirred solution of 243-2 (400.0 mg, 0.93 mmol) in CH₂Cl₂ (10 mL) was added TFA (5 mL). The reaction mixture was stirred at room temperature for 3 hours until the reaction was complete (by LCMS). The mixture was concentrated under reduced pressure to afford 243-3 (200 mg, yield: 65%) as yellow oil, which was used to the next step without purification.

### The Synthesis of 4-(4-((4-(methylsulfonyl)benzyl)oxy)phenyl)-N-(4-phenylbutyl)-1H-imidazole-1-carboxamide (AC2020601-0243)

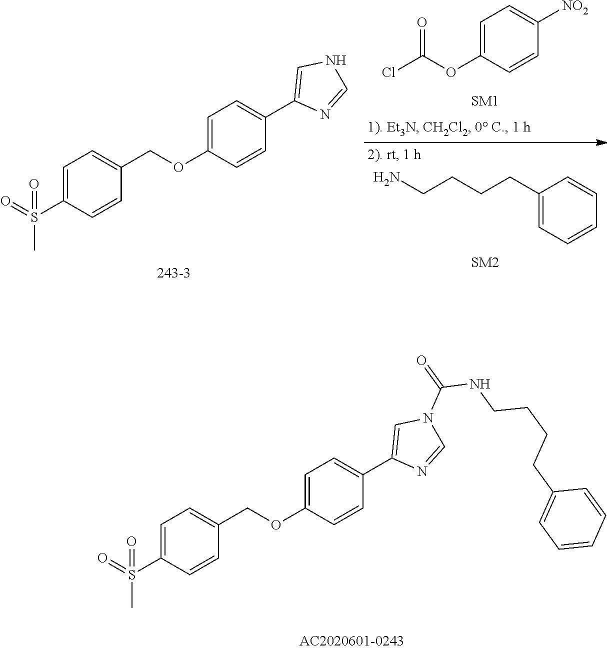 [SMILES: CS(=O)(=O)c1ccc(COc2ccc(-c3c[nH]cn3)cc2)cc1 ; O=C(Cl)Oc1ccc([N+](=O)[O-])cc1 ; NCCCCc1ccccc1 ; CS(=O)(=O)c1ccc(COc2ccc(-c3cn(C(=O)NCCCCc4ccccc4)cn3)cc2)cc1]

To a solution of 243-3 (131 mg, 0.4 mmol) in DCM (5 mL) was added SM1 (82.0 mg, 0.4 mmol) and Et₃N (5 mL) at 0° C. and stirred for 1 hour. Then the reaction mixture was allowed to warm to room temperature and SM2 (60.0 mg, 0.4 mmol) was added. After 1 hour stirring, the reaction mixture was quenched with water, extracted with DCM (20 mL), the combined organic layer was washed with brine, dried over anhydrous Na₂SO₄ and evaporated under reduced pressure. The crude was purified by silica gel chromatography (10% MeOH/DCM) to afford AC2020601-0243 (79 mg, yield: 39%) as a white solid. ¹H NMR (400 MHz, DMSO-d₆) δ 8.46 (t, J=5.6 Hz, 1H), 8.23 (d, J=0.8 Hz, 1H), 7.97 (d, J=1.2 Hz, 1H), 7.93 (d, J=8.4 Hz, 2H), 7.68-7.71 (m, 4H), 7.23-7.26 (m, 2H), 7.12-7.19 (m, 3H), 7.03 (d, J=8.8 Hz, 2H), 5.25 (s, 2H), 3.23-3.28 (m, 2H), 3.19 (s, 3H), 2.59 (t, J=7.2 Hz, 2H), 1.51-1.63 (m, 4H). LC-MS m/z: 503.9 [M+H]⁺. HPLC Purity (254 nm): 99.40%; t_{R}=8.495 min.

AC2020601-0245

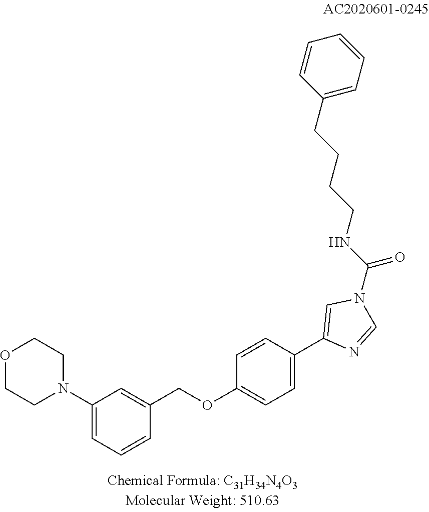 [SMILES: O=C(NCCCCc1ccccc1)n1cnc(-c2ccc(OCc3cccc(N4CCOCC4)c3)cc2)c1]

### The Synthesis of tert-butyl 4-(4-((3-morpholinobenzyl)oxy)phenyl)-1H-imidazole-1-carboxylate (245-2)

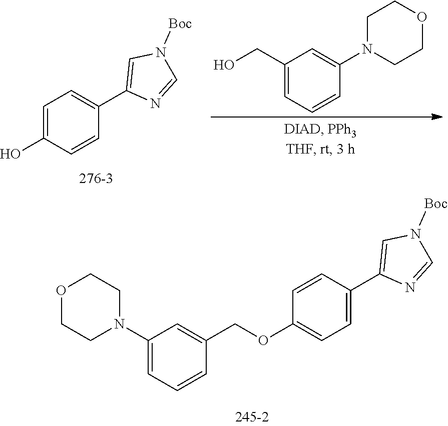 [SMILES: CC(C)(C)OC(=O)n1cnc(-c2ccc(O)cc2)c1 ; OCc1cccc(N2CCOCC2)c1 ; CC(C)(C)OC(=O)n1cnc(-c2ccc(OCc3cccc(N4CCOCC4)c3)cc2)c1]

To a suspension of 276-3 (780.0 mg, 3.0 mmol) in THF (10 mL) was added (4-(methylsulfonyl)phenyl)methanol (579.0 mg, 3.0 mmol), PPh₃ (942.0 mg, 3.6 mmol) and DIAD (789.0 mg, 3.9 mmol). The reaction mixture was stirred at room temperature for 3 hours until the reaction was complete (by LCMS). Saturated NaCl solution was added and the reaction mixture was extracted with EtOAc (20 mL), the combined organic layer was washed with brine, dried over anhydrous Na₂SO₄ and evaporated under reduced pressure. The crude was purified by silica gel chromatography (10% MeOH/DCM) to afford 245-2 (1.01 g, yield: 77%) as yellow oil.

### The Synthesis of 4-(3-((4-(1H-imidazol-4-yl)phenoxy)methyl)phenyl)morpholine (245-3)

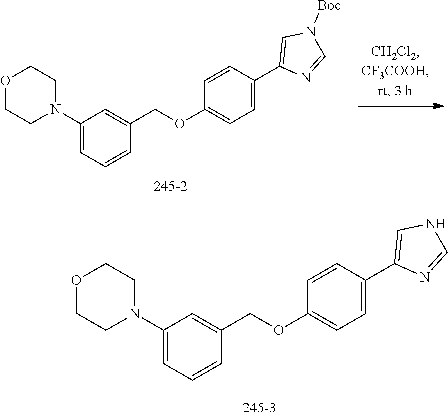 [SMILES: CC(C)(C)OC(=O)n1cnc(-c2ccc(OCc3cccc(N4CCOCC4)c3)cc2)c1 ; c1cc(COc2ccc(-c3c[nH]cn3)cc2)cc(N2CCOCC2)c1]

To a stirred solution of 245-2 (500.0 mg, 1.15 mmol) in CH₂Cl₂ (10 mL) was added TFA (5 mL). The reaction mixture was stirred at room temperature for 3 hours until the reaction was complete (by LCMS). The mixture was concentrated under reduced pressure to afford 245-3 (300 mg, yield: 78%) as yellow oil, which was used to the next step without purification.

### The Synthesis of 4-(4-((3-morpholinobenzyl)oxy)phenyl)-N-(4-phenylbutyl)-1H-imidazole-1-carboxamide (AC2020601-0245)

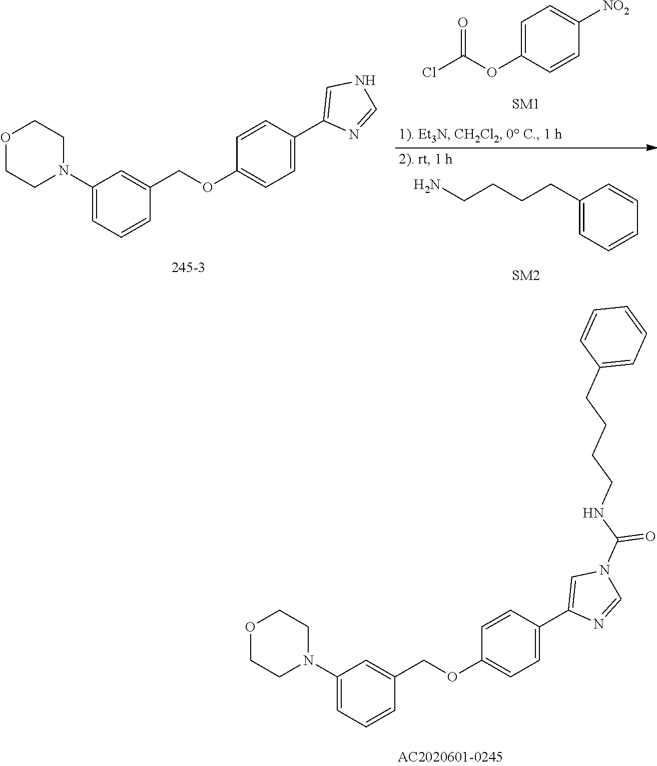 [SMILES: O=C(Cl)Oc1ccc([N+](=O)[O-])cc1 ; c1cc(COc2ccc(-c3c[nH]cn3)cc2)cc(N2CCOCC2)c1 ; NCCCCc1ccccc1 ; O=C(NCCCCc1ccccc1)n1cnc(-c2ccc(OCc3cccc(N4CCOCC4)c3)cc2)c1]

To a solution of 245-3 (134 mg, 0.4 mmol) in DCM (5 mL) was added SM1 (82.0 mg, 0.4 mmol) and Et₃N (1 mL) at 0° C. and stirred for 1 hour. Then the reaction mixture was allowed to warm to room temperature and SM2 (60.0 mg, 0.4 mmol) was added. After 1 hour stirring, the reaction mixture was quenched with water, extracted with DCM (20 mL), the combined organic layer was washed with brine, dried over anhydrous Na₂SO₄ and evaporated under reduced pressure. The crude was purified by silica gel chromatography (10% MeOH/DCM) to afford AC2020601-0245 (45 mg, yield: 22%) as a white solid. ¹H NMR (400 MHz, CDCl₃) δ 8.95 (s, 1H), 7.70 (d, J=8.8 Hz, 2H), 7.58 (s, 1H), 7.27-7.30 (m, 4H), 7.17-7.20 (m, 3H), 6.98-7.02 (m, 3H), 6.93 (d, J=7.2 Hz, 1H), 6.88 (dd, J=8.4 Hz, 2.4 Hz, 1H), 5.04 (s, 2H), 3.85 (t, J=4.8 Hz, 4H), 3.46-3.48 (m, 2H), 3.17 (t, J=4.8 Hz, 4H), 2.67 (t, J=6.8 Hz, 2H), 1.72-1.74 (m, 4H). LC-MS m/z: 511.0 [M+H]⁺. HPLC Purity (254 nm): 96.09%; t_{R}=8.818 min.

AC2020601-0246

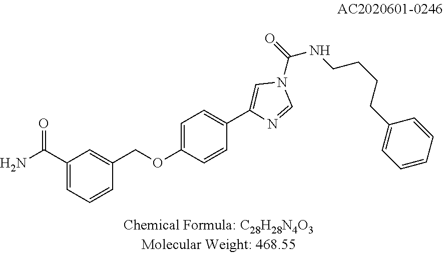 [SMILES: NC(=O)c1cccc(COc2ccc(-c3cn(C(=O)NCCCCc4ccccc4)cn3)cc2)c1]

### The Synthesis of tert-butyl 4-(4-((3-cyanobenzyl)oxy)phenyl)-1H-imidazole-1-carboxylate (246-2)

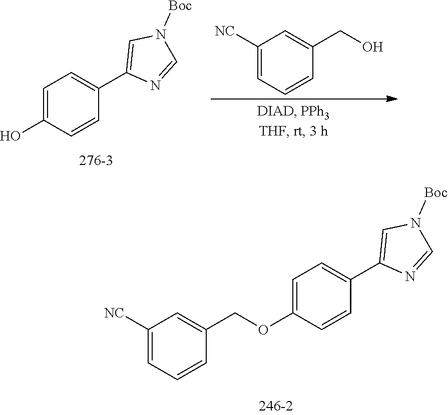 [SMILES: CC(C)(C)OC(=O)n1cnc(-c2ccc(O)cc2)c1 ; N#Cc1cccc(CO)c1 ; CC(C)(C)OC(=O)n1cnc(-c2ccc(OCc3cccc(C#N)c3)cc2)c1]

To a suspension of 276-3 (780.0 mg, 3.0 mmol) in THF (10 mL) was added 3-(hydroxymethyl)benzonitrile (399.5 mg, 3.0 mmol), PPh₃ (942.0 mg, 3.6 mmol) and DIAD (789.0 mg, 3.9 mmol). The reaction mixture was stirred at room temperature for 3 hours until the reaction was complete (by LCMS). Saturated NaCl solution was added and the reaction mixture was extracted with EtOAc (20 mL), the combined organic layer was washed with brine, dried over anhydrous Na₂SO₄ and evaporated under reduced pressure. The crude was purified by silica gel chromatography (10% MeOH/DCM) to afford 246-2 (810 mg, yield: 72%) as yellow oil.

### The Synthesis of 4-(3-((4-(1H-imidazol-4-yl)phenoxy)methyl)phenyl)morpholine (246-3)

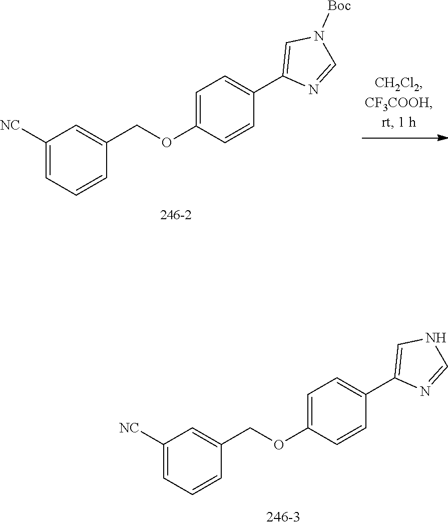 [SMILES: CC(C)(C)OC(=O)n1cnc(-c2ccc(OCc3cccc(C#N)c3)cc2)c1 ; N#Cc1cccc(COc2ccc(-c3c[nH]cn3)cc2)c1]

To a stirred solution of 246-2 (600 mg, 1.60 mmol) in CH₂Cl₂ (10 mL) was added TFA (5 mL). The reaction mixture was stirred at room temperature for 3 hours until the reaction was complete (by LCMS). The mixture was concentrated under reduced pressure to afford 246-3 (430 mg, yield: 98%) as yellow oil, which was used to the next step without purification.

### The Synthesis of 3-((4-(1H-imidazol-4-yl)phenoxy)methyl)benzamide (246-4)

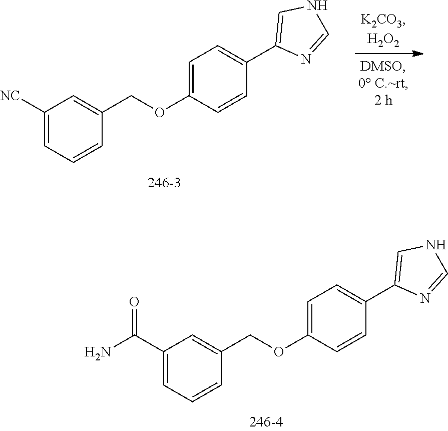 [SMILES: N#Cc1cccc(COc2ccc(-c3c[nH]cn3)cc2)c1 ; NC(=O)c1cccc(COc2ccc(-c3c[nH]cn3)cc2)c1]

To a solution of 246-3 (200 mg, 0.73 mmol) in DMSO (5 mL) was added K₂CO₃ (200 mg, 1.45 mmol) and stirred for 1 hour. Then the reaction mixture was allowed to 0° C. and H₂O₂ (1 ml, 30% in water) was added. After 1 hour stirring, the reaction mixture was quenched with water, extracted with EA (20 mL), the combined organic layer was washed with brine, dried over anhydrous Na₂SO₄. The mixture was concentrated under reduced pressure to afford 246-4 (150 mg, yield: 70%) as a yellow solid, which was used to the next step without purification.

### The Synthesis of 4-(4-((3-carbamoylbenzyl)oxy)phenyl)-N-(4-phenylbutyl)-1H-imidazole-1-carboxamide (AC2020601-0246)

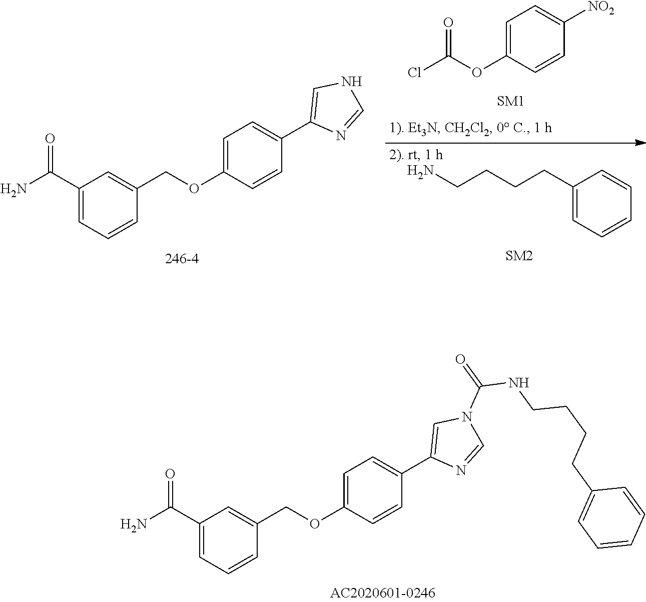 [SMILES: O=C(Cl)Oc1ccc([N+](=O)[O-])cc1 ; NC(=O)c1cccc(COc2ccc(-c3c[nH]cn3)cc2)c1 ; NCCCCc1ccccc1 ; NC(=O)c1cccc(COc2ccc(-c3cn(C(=O)NCCCCc4ccccc4)cn3)cc2)c1]

To a solution of 246-4 (117 mg, 0.4 mmol) in DCM (5 mL) was added SM1 (82.0 mg, 0.4 mmol) and Et₃N (1 mL) at 0° C. and stirred for 1 hour. Then the reaction mixture was allowed to warm to room temperature and SM2 (60.0 mg, 0.4 mmol) was added. After 1 hour stirring, the reaction mixture was quenched with water, extracted with DCM (20 mL), the combined organic layer was washed with brine, dried over anhydrous Na₂SO₄ and evaporated under reduced pressure. The crude was purified by silica gel chromatography (10% MeOH/DCM) to afford AC2020601-0246 (6 mg, yield: 3%) as a white solid. ¹H NMR (400 MHz, DMSO-d₆) δ 8.55 (s, 1H), 8.25 (s, 1H), 8.01 (s, 2H), 7.95 (s, 1H), 7.81 (d, J=8.0 Hz, 1H), 7.69 (d, J=8.4 Hz, 2H), 7.58 (d, J=7.2 Hz, 1H), 7.43-7.47 (m, 1H), 7.39 (s, 1H), 7.23-7.26 (m, 2H), 7.14-7.19 (m, 3H), 7.03 (d, J=8.8 Hz, 2H), 5.14 (s, 2H), 3.23-3.28 (m, 2H), 2.59 (t, J=8.0 Hz, 2H), 1.52-1.63 (m, 4H). LC-MS m/z: 469.0 [M+H]⁺. HPLC Purity (214 nm): 96.41%; t_{R}=7.965 min.

AC2020601-0275

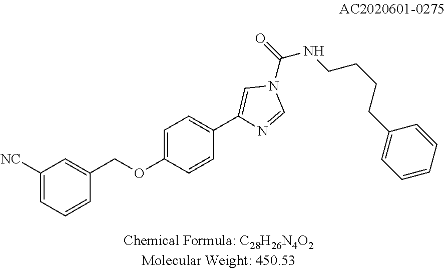 [SMILES: N#Cc1cccc(COc2ccc(-c3cn(C(=O)NCCCCc4ccccc4)cn3)cc2)c1]

### The Synthesis of 4-(4-((3-cyanobenzyl)oxy)phenyl)-N-(4-phenylbutyl)-1H-imidazole-1-carboxamide (AC2020601-0275)

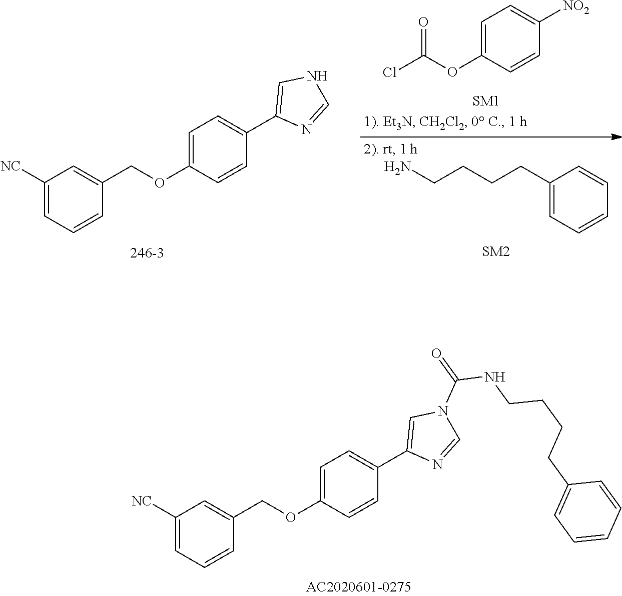 [SMILES: O=C(Cl)Oc1ccc([N+](=O)[O-])cc1 ; N#Cc1cccc(COc2ccc(-c3c[nH]cn3)cc2)c1 ; NCCCCc1ccccc1 ; N#Cc1cccc(COc2ccc(-c3cn(C(=O)NCCCCc4ccccc4)cn3)cc2)c1]

To a solution of 246-3 (110 mg, 0.4 mmol) in DCM (5 mL) was added SM1 (82.0 mg, 0.4 mmol) and Et₃N (1 mL) at 0° C. and stirred for 1 hour. Then the reaction mixture was allowed to warm to room temperature and SM2 (60.0 mg, 0.4 mmol) was added. After 1 hour stirring, the reaction mixture was quenched with water, extracted with DCM (20 mL), the combined organic layer was washed with brine, dried over anhydrous Na₂SO₄ and evaporated under reduced pressure. The crude was purified by silica gel chromatography (10% MeOH/DCM) to afford AC2020601-0275 (22 mg, yield: 12%) as a pink solid. ¹H NMR (400 MHz, CDCl₃) δ 8.72 (s, 1H), 7.70-7.74 (m, 3H), 7.66 (d, J=7.6 Hz, 1H), 7.62 (d, J=7.6 Hz, 1H), 7.57 (s, 1H), 7.48-7.52 (m, 1H), 7.25-7.29 (m, 2H), 7.17-7.20 (m, 3H), 7.98 (d, J=8.8 Hz, 2H), 6.66 (s, 1H), 5.10 (s, 2H), 3.46-3.47 (m, 2H), 2.67 (t, J=7.2 Hz, 2H), 1.71-1.73 (m, 4H). LC-MS m/z: 451.0 [M+H]⁺. HPLC Purity (254 nm): 95.60%; t_{R}=10.321 min.

AC2020601-0276

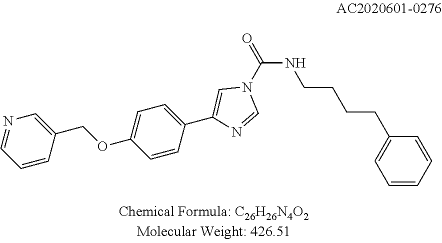 [SMILES: O=C(NCCCCc1ccccc1)n1cnc(-c2ccc(OCc3cccnc3)cc2)c1]

### The Synthesis of 4-(1H-imidazol-4-yl)phenol (276-2)

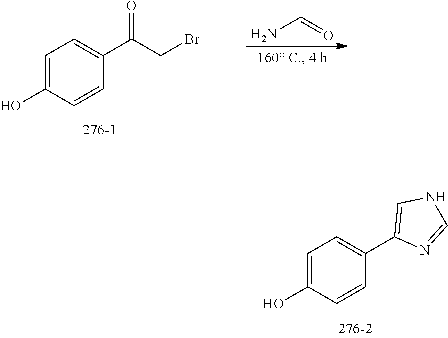

A stirred solution of 276-1 (20.0 g, 93.0 mmol) in formamide (50 mL) was heated to 160° C. for 4 hour. Then the reaction mixture was poured into water (20 mL), extracted with EtOAc (20 mL), the combined organic layer was washed with water and brine, dried over anhydrous Na₂SO₄, filtered and evaporated under reduced pressure. The crude was purified by silica gel chromatography (5-20% MeOH in CH₂Cl₂) to get 276-2 (10.0 g, yield: 71.4%) as a brown solid.

### The Synthesis of tert-butyl 4-(4-hydroxyphenyl)-1H-imidazole-1-carboxylate (276-3)

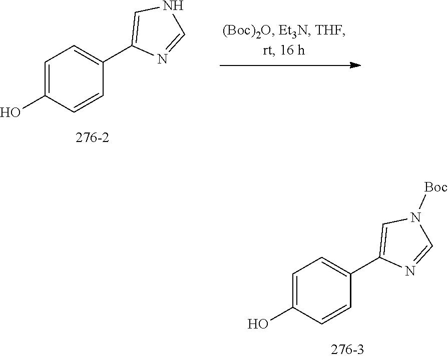 [SMILES: Oc1ccc(-c2c[nH]cn2)cc1 ; CC(C)(C)OC(=O)n1cnc(-c2ccc(O)cc2)c1]

To a suspension of 276-2 (10 g, 62.5 mmol) in THF (50 mL) was added Et₃N (12.6 g, 125 mmol) and (Boc)₂O (9.5 g, 43.8 mmol) at 0° C. The reaction mixture was allowed to warm to room temperature and stirred for 16 hours until the reaction was complete (by LCMS). The reaction mixture was added EtOAc and the precipitate was collected to afford 276-3 (12.0 g, yield: 74%) as a white solid.

### The Synthesis of tert-butyl 4-(4-(pyridin-3-ylmethoxy)phenyl)-1H-imidazole-1-carboxylate (276-4)

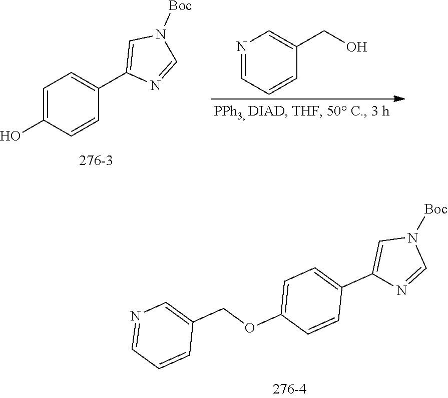 [SMILES: CC(C)(C)OC(=O)n1cnc(-c2ccc(O)cc2)c1 ; OCc1cccnc1 ; CC(C)(C)OC(=O)n1cnc(-c2ccc(OCc3cccnc3)cc2)c1]

To a suspension of 276-3 (780.0 mg, 3.0 mmol) in THF (10 mL) was added pyridin-3-ylmethanol (327.0 mg, 3.0 mmol), PPh₃ (942.0 mg, 3.6 mmol) and DIAD (789.0 mg, 3.9 mmol). The reaction mixture was allowed to warm to 50° C. for 3 hours until the reaction was complete (by LCMS). Saturated NaCl solution was added and the reaction mixture was extracted with EtOAc (20 mL), the combined organic layer was washed with brine, dried over anhydrous Na₂SO₄ and evaporated under reduced pressure. The crude was purified by silica gel chromatography (10% MeOH/DCM) to afford 276-4 (790.0 mg, yield: 75%) as yellow oil.

### The Synthesis of 3-((4-(1H-imidazol-4-yl)phenoxy)methyl)pyridine (276-5)

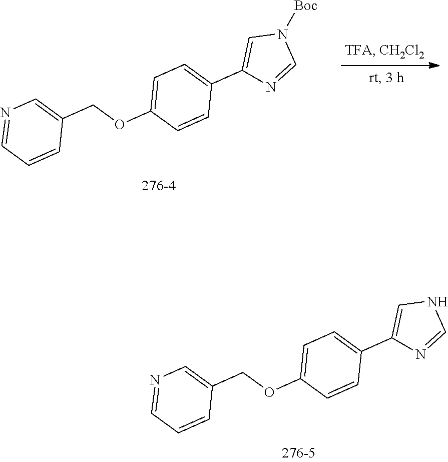 [SMILES: CC(C)(C)OC(=O)n1cnc(-c2ccc(OCc3cccnc3)cc2)c1 ; c1cncc(COc2ccc(-c3c[nH]cn3)cc2)c1]

To a stirred solution of 276-4 (280.0 mg, 0.8 mmol) in CH₂Cl₂ (10 mL) was added TFA (5 mL). The reaction mixture was stirred at room temperature for 3 hours until the reaction was complete (by LCMS). The mixture was concentrated under reduced pressure to afford 276-5 (100 mg, yield: 50%) as yellow oil, which was used to the next step without purification.

### The Synthesis of N-(4-phenylbutyl)-4-(4-(pyridin-3-ylmethoxy)phenyl)-1H-imidazole-1-carboxamide (AC2020601-0276)

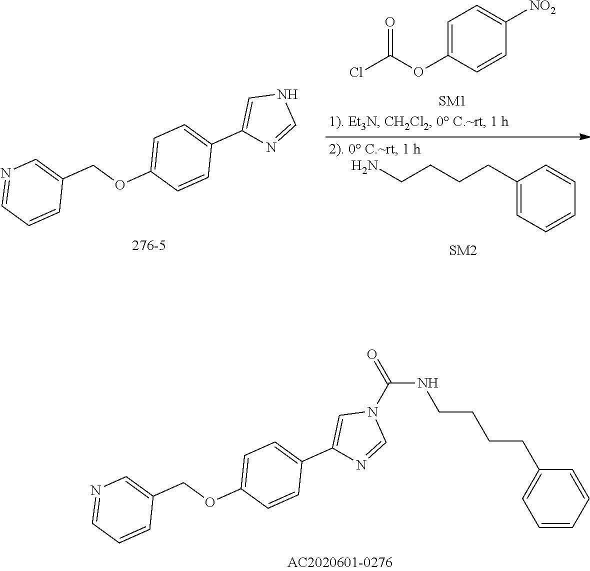 [SMILES: O=C(Cl)Oc1ccc([N+](=O)[O-])cc1 ; c1cncc(COc2ccc(-c3c[nH]cn3)cc2)c1 ; NCCCCc1ccccc1 ; O=C(NCCCCc1ccccc1)n1cnc(-c2ccc(OCc3cccnc3)cc2)c1]

To a solution of 276-5 (100 mg, 0.4 mmol) in DCM (5 mL) was added SM1 (82.0 mg, 0.4 mmol) and Et₃N (5 mL) at 0° C. and stirred for 1 hour. Then the reaction mixture was allowed to warm to rt and SM2 (60.0 mg, 0.4 mmol) was added. After 1 hour stirring, the reaction mixture was quenched with water, extracted with DCM (20 mL), the combined organic layer was washed with brine, dried over anhydrous Na₂SO₄ and evaporated under reduced pressure. The crude was purified by silica gel chromatography (10% MeOH/DCM) to afford AC2020601-0276 (13.2 mg, yield: 7.6%) as a white solid. ¹H NMR (400 MHz, CDCl₃) δ 9.29 (s, 1H), 8.71 (s, 1H), 8.61 (s, 1H), 7.82 (d, J=8.0 Hz, 1H), 7.76 (d, J=8.8 Hz, 2H), 7.67 (s, 1H), 7.36-7.39 (m, 1H), 7.26-7.30 (m, 3H), 7.17-7.20 (m, 3H), 7.03 (d, J=8.8 Hz, 2H), 5.12 (s, 2H), 3.43-3.55 (m, 2H), 2.68 (t, J=7.2 Hz, 2H), 1.71-1.83 (m, 4H). LC-MS m/z: 427.0 [M+H]⁺. HPLC Purity (254 nm): 99.30%; t_{R}=7.139 min.

AC2020601-0278

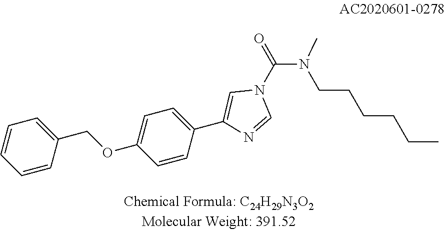 [SMILES: CCCCCCN(C)C(=O)n1cnc(-c2ccc(OCc3ccccc3)cc2)c1]

### The Synthesis of 1-(4-(benzyloxy)phenyl)ethanone (0149-2)

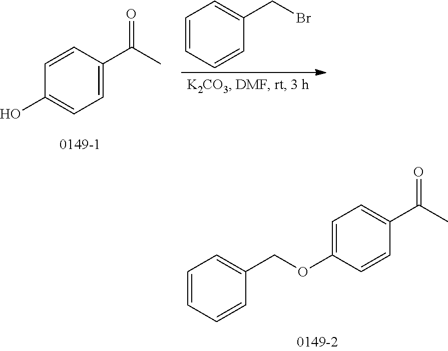

To a stirred solution of 0149-1 (680 mg, 5.0 mmol) in DMF (20 mL) was added K₂CO₃ (1.38 g 10.0 mmol) and (bromomethyl)benzene (0.6 mL, 5.0 mmol), and the reaction mixture was allowed to stir at room temperature for 3 hours. After the reaction finished (by LCMS), water (50 mL) was added, extracted with EtOAc, the combined organic layer was washed with brine, dried over anhydrous Na₂SO₄, filtered and evaporated under reduced pressure. The crude was recrystallized with CH₂Cl₂ and hexane to afford 0149-2 (1.09 g, yield: 96.5%) as a white solid. LC-MS t_{R}=1.875 min.

### The Synthesis of 1-(4-(benzyloxy)phenyl)-2-bromoethanone (0149-3)

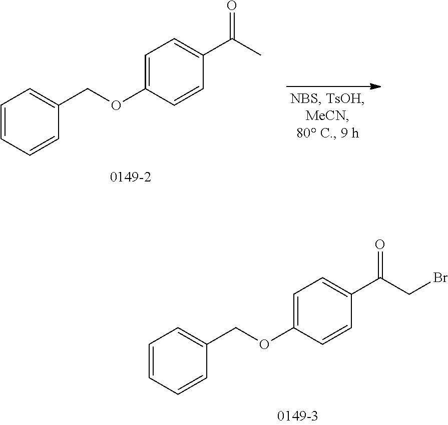 [SMILES: CC(=O)c1ccc(OCc2ccccc2)cc1 ; O=C(CBr)c1ccc(OCc2ccccc2)cc1]

To a stirred solution of 0149-2 (1.09 g, 4.8 mmol) in MeCN (30 mL) was added TsOH (90 mg, 0.5 mmol) and NBS (855 mg, 4.8 mmol), and this mixture was heated to 80° C. for 9 hours. After the reaction was completed (by LCMS), the mixture was concentrated under reduced pressure and dissolved in EtOAc (50 mL). The residue was washed with water and brine, dried over anhydrous Na₂SO₄, filtered and evaporated under reduced pressure. The crude was purified by prep-HPLC to give 0149-3 (780 mg, yield: 53.4%) as a white solid. LC-MS t_{R}=1.964 min.

### The Synthesis of 4-(4-(benzyloxy)phenyl)-1H-imidazole (0149-4)

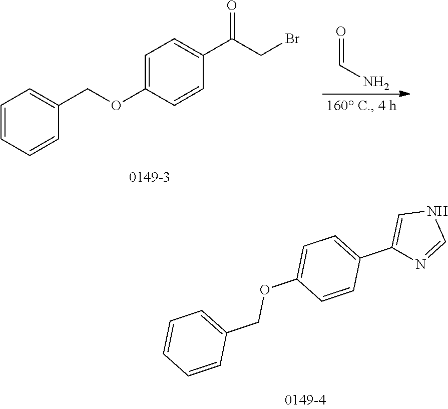

A stirred solution of PJ2-0149-3 (400 mg, 1.31 mmol) in formamide (5 mL) was heated to 160° C. for 4 hours. Then the reaction mixture was poured into water (20 mL), extracted with EtOAc, the combined organic layer was washed with water and brine, dried over anhydrous Na₂SO₄, filtered and evaporated under reduced pressure. The crude was purified by silica gel chromatography (5-20% MeOH in CH₂Cl₂) to get 0149-4 (310 mg, yield: 94.7%) as a pink solid.

### The Synthesis of 4-(4-(benzyloxy)phenyl)-N-methyl-N-(4-phenylbutyl)-1H-imidazole-1-carboxamide (AC2020601-0278)

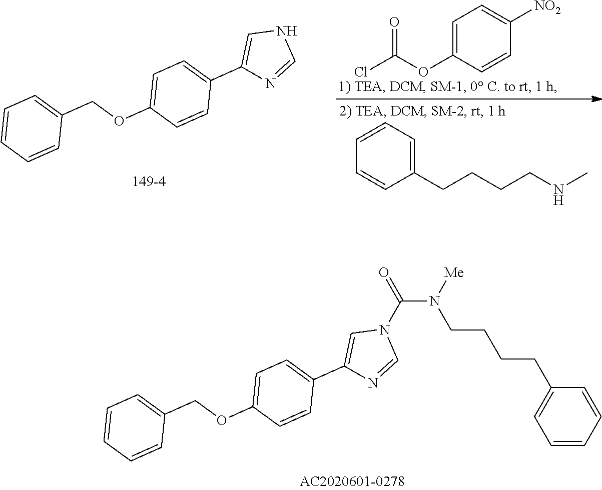 [SMILES: c1ccc(COc2ccc(-c3c[nH]cn3)cc2)cc1 ; CNCCCCc1ccccc1 ; O=C(Cl)Oc1ccc([N+](=O)[O-])cc1 ; CN(CCCCc1ccccc1)C(=O)n1cnc(-c2ccc(OCc3ccccc3)cc2)c1]

A mixture of 149-4 (300 mg, 1.20 mmol) and TEA (242 mg, 2.40 mmol) in CH₂Cl₂ (20 mL) was added 4-nitrophenyl carbonochloridate (266 mg, 1.32 mmol), and the reaction mixture was stirred at room temperature for 1 h. N-methyl-4-phenylbutan-1-amine (215 mg, 1.32 mmol) was added to the reaction, the mixture was stirred at room temperature for another 1 h. After the reaction was completed (by LCMS), the mixture was treated with brine (50 mL) and extracted with CH₂Cl₂ (100 mL×2), dried over anhydrous sodium sulfate, concentrated in vacuo to give the crude product, then it was purified by CC to give AC2020601-0278 (82 mg, yield: 16%) as a white solid. ¹H NMR (400 MHz, CDCl₃) δ 7.98 (s, 1H), 7.71-7.75 (m, 2H), 7.44-7.46 (m, 2H), 7.38-7.42 (m, 3H), 7.33-7.35 (m, 1H), 7.26-7.31 (m, 2H), 7.16-7.22 (m, 3H), 7.01-7.04 (m, 2H), 5.10 (s, 2H), 3.47 (t, J=7.6 Hz, 2H), 3.08 (s, 3H), 2.66 (t, J=6.8 Hz, 2H), 1.64-1.74 (m, 4H). LC-MS m/z: 440.0 [M+H]⁺. HPLC Purity (254 nm): 98.9%; t_{R}=9.465 min.

AC2020601-0284

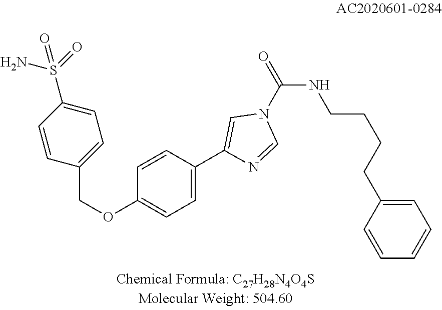 [SMILES: NS(=O)(=O)c1ccc(COc2ccc(-c3cn(C(=O)NCCCCc4ccccc4)cn3)cc2)cc1]

### The Synthesis of tert-butyl 4-(4-((4-sulfamoylbenzyl)oxy)phenyl)-1H-imidazole-1-carboxylate (284-3)

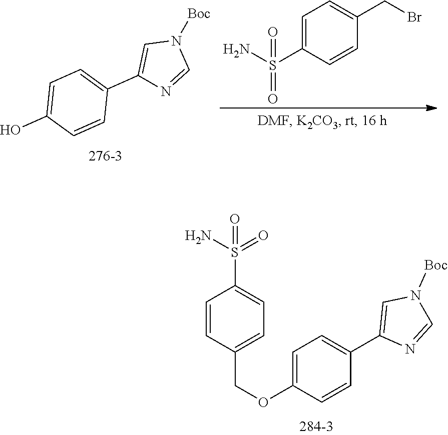 [SMILES: CC(C)(C)OC(=O)n1cnc(-c2ccc(O)cc2)c1 ; NS(=O)(=O)c1ccc(CBr)cc1 ; CC(C)(C)OC(=O)n1cnc(-c2ccc(OCc3ccc(S(N)(=O)=O)cc3)cc2)c1]

To a suspension of 276-3 (520 mg, 2.00 mmol) in DMF (10 mL) was added K₂CO₃ (552 mg, 4.00 mmol) and 4-(bromomethyl)benzenesulfonamide (600 mg, 2.40 mmol). The reaction mixture was stirred at room temperature for 16 hours until the reaction was complete (by LCMS). Saturated NaCl solution was added and the reaction mixture was extracted with EtOAc (50 mL), the combined organic layer was washed with brine, dried over anhydrous Na₂SO₄ and evaporated under reduced pressure. The crude was purified by silica gel chromatography (5% MeOH/DCM) to afford 284-3 (500 mg, yield: 58%) as a yellow solid.

### The Synthesis of 4-((4-(1H-imidazol-4-yl)phenoxy)methyl)benzenesulfonamide (284-4)

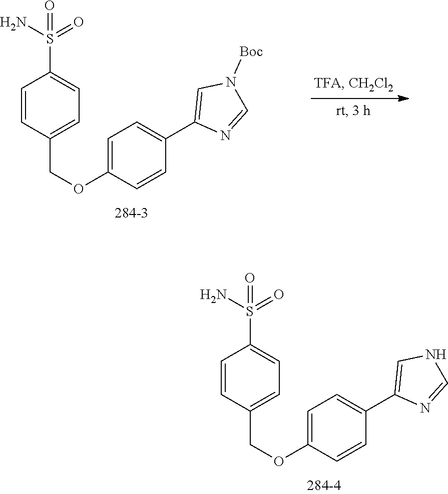 [SMILES: CC(C)(C)OC(=O)n1cnc(-c2ccc(OCc3ccc(S(N)(=O)=O)cc3)cc2)c1 ; NS(=O)(=O)c1ccc(COc2ccc(-c3c[nH]cn3)cc2)cc1]

To a stirred solution of 284-3 (500 mg, 1.16 mmol) in CH₂Cl₂ (10 mL) was added TFA (5 mL). The reaction mixture was stirred at room temperature for 3 hours until the reaction was complete (by LCMS). The mixture was concentrated under reduced pressure to afford 284-4 (200 mg, yield: 52%) as yellow oil, which was used to the next step without purification.

### The Synthesis of N-(4-phenylbutyl)-4-(4-((4-sulfamoylbenzyl)oxy)phenyl)-1H-imidazole-1-carboxamide (AC2020601-0284)

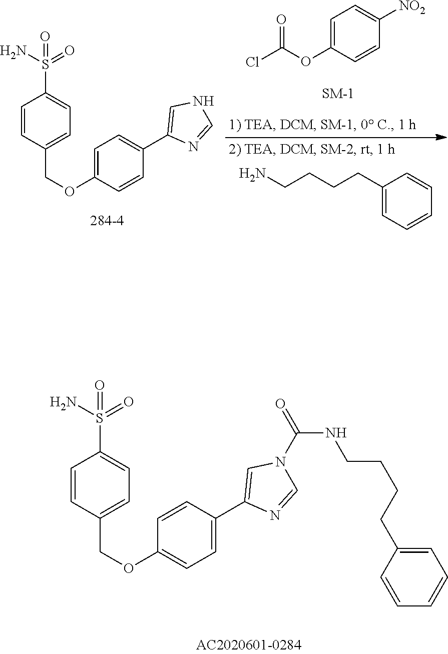 [SMILES: NS(=O)(=O)c1ccc(COc2ccc(-c3c[nH]cn3)cc2)cc1 ; O=C(Cl)Oc1ccc([N+](=O)[O-])cc1 ; NCCCCc1ccccc1 ; NS(=O)(=O)c1ccc(COc2ccc(-c3cn(C(=O)NCCCCc4ccccc4)cn3)cc2)cc1]

To a solution of 284-4 (200 mg, 0.61 mmol) in DCM (5 mL) was added 4-nitrophenyl carbonochloridate (147 mg, 0.73 mmol) and Et₃N (5 mL) at 0° C. and stirred for 1 hour. Then the reaction mixture was allowed to warm to room temperature and 4-phenylbutan-1-amine (109 mg, 0.73 mmol) was added. After 1 hour stirring, the reaction mixture was quenched with water, extracted with DCM (20 mL), the combined organic layer was washed with brine, dried over anhydrous Na₂SO₄ and evaporated under reduced pressure. The crude was purified by silica gel chromatography (5% MeOH/DCM) to afford AC2020601-0284 (18 mg, yield: 6%) as a white solid. ¹H NMR (400 MHz, DMSO-d₆) δ 8.48 (t, J=5.6 Hz, 1H), 8.25 (d, J=1.2 Hz, 1H), 7.99 (d, J=0.8 Hz, 1H), 7.84 (d, J=8.0 Hz, 2H), 7.72 (d, J=8.8 Hz, 2H), 7.64 (d, J=8.4 Hz, 2H), 7.26 (s, 2H), 7.26-7.29 (m, 2H), 7.17-7.22 (m, 3H), 7.05 (d, J=8.8 Hz, 2H), 5.23 (s, 2H), 3.26-3.31 (m, 2H), 2.62 (t, J=7.6 Hz, 2H), 1.55-1.66 (m, 4H). LC-MS m/z: 505.0 [M+H]⁺. HPLC Purity (254 nm): 96.40%; t_{R}=9.258 min.

AC2020601-0294

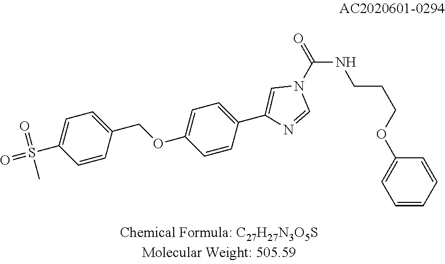 [SMILES: CS(=O)(=O)c1ccc(COc2ccc(-c3cn(C(=O)NCCCOc4ccccc4)cn3)cc2)cc1]

### The Synthesis of tert-butyl 4-(4-((4-(methylsulfonyl)benzyl)oxy)phenyl)-1H-imidazole-1-carboxylate (243-2)

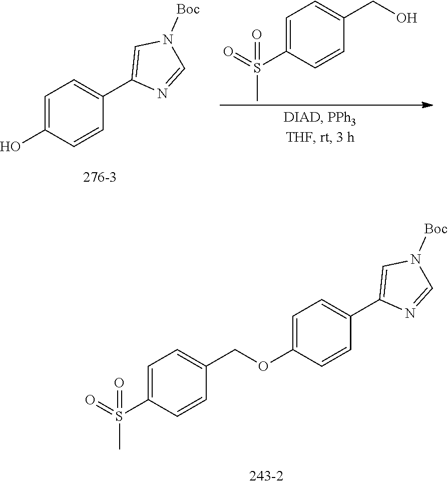 [SMILES: CC(C)(C)OC(=O)n1cnc(-c2ccc(O)cc2)c1 ; CS(=O)(=O)c1ccc(CO)cc1 ; CC(C)(C)OC(=O)n1cnc(-c2ccc(OCc3ccc(S(C)(=O)=O)cc3)cc2)c1]

To a suspension of 276-3 (780 mg, 3.0 mmol) in THF (10 mL) was added (4-(methylsulfonyl)phenyl)methanol (558 mg, 3.0 mmol), PPh₃ (942 mg, 3.6 mmol) and DIAD (789 mg, 3.9 mmol). The reaction mixture was stirred at room temperature for 3 hours until the reaction was complete (by LCMS). Saturated NaCl solution was added and the reaction mixture was extracted with EtOAc (20 mL), the combined organic layer was washed with brine, dried over anhydrous Na₂SO₄ and evaporated under reduced pressure. The crude was purified by silica gel chromatography (10% MeOH/DCM) to afford 243-2 (680 mg, yield: 53%) as yellow oil.

### The Synthesis of 4-(4-((4-(methylsulfonyl)benzyl)oxy)phenyl)-1H-imidazole (243-3)

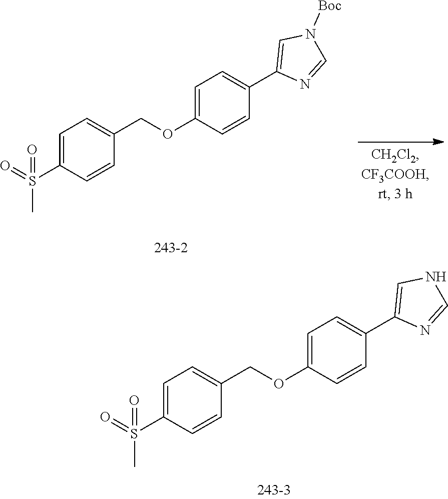 [SMILES: CC(C)(C)OC(=O)n1cnc(-c2ccc(OCc3ccc(S(C)(=O)=O)cc3)cc2)c1 ; CS(=O)(=O)c1ccc(COc2ccc(-c3c[nH]cn3)cc2)cc1]

To a stirred solution of 243-2 (400 mg, 0.93 mmol) in CH₂Cl₂ (10 mL) was added TFA (5 mL). The reaction mixture was stirred at room temperature for 3 hours until the reaction was complete (by LCMS). The mixture was concentrated under reduced pressure to afford 243-3 (200 mg, yield: 65%) as yellow oil, which was used to the next step without purification.

### The Synthesis of 4-(4-((4-(methylsulfonyl)benzyl)oxy)phenyl)-N-(3-phenoxypropyl)-1H-imidazole-1-carboxamide (AC2020601-0294)

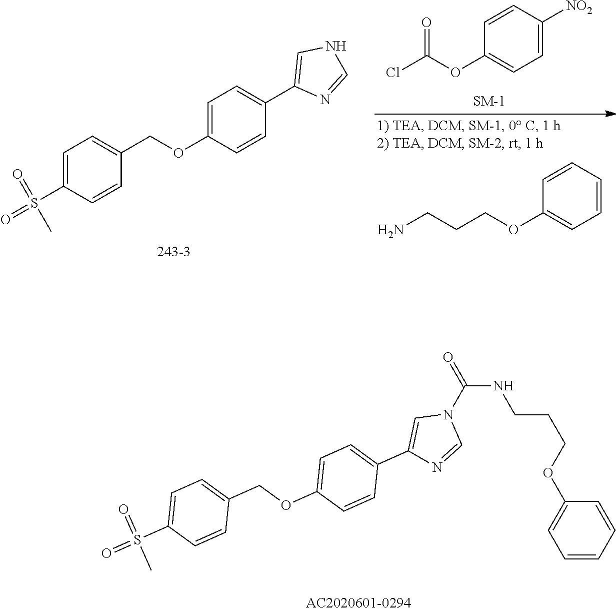 [SMILES: CS(=O)(=O)c1ccc(COc2ccc(-c3c[nH]cn3)cc2)cc1 ; O=C(Cl)Oc1ccc([N+](=O)[O-])cc1 ; NCCCOc1ccccc1 ; CS(=O)(=O)c1ccc(COc2ccc(-c3cn(C(=O)NCCCOc4ccccc4)cn3)cc2)cc1]

To a solution of 243-3 (100 mg, 0.30 mmol) in DCM (5 mL) was added 4-nitrophenyl carbonochloridate (74 mg, 0.37 mmol) and Et₃N (5 mL) at 0° C. and stirred for 1 hour. Then the reaction mixture was allowed to warm to room temperature and 3-phenoxypropan-1-amine (55 mg, 0.37 mmol) was added. After 1 hour stirring, the reaction mixture was quenched with water, extracted with DCM (20 mL), the combined organic layer was washed with brine, dried over anhydrous Na₂SO₄ and evaporated under reduced pressure. The crude was purified by silica gel chromatography (5% MeOH/DCM) to afford AC2020601-0294 (13 mg, yield: 8%) as a white solid. ¹H NMR (400 MHz, DMSO-d₆) δ 8.59 (t, J=5.6 Hz, 1H), 8.27 (d, J=1.2 Hz, 1H), 8.01 (d, J=1.6 Hz, 1H), 7.96 (d, J=8.4 Hz, 2H), 7.71-7.75 (m, 4H), 7.27-7.31 (m, 2H), 7.07 (d, J=8.8 Hz, 2H), 6.91-6.95 (m, 3H), 5.28 (s, 2H), 4.06 (d, J=6.0 Hz, 2H), 3.42-3.47 (m, 2H), 3.22 (s, 3H), 2.00-2.03 (m, 2H). LC-MS m/z: 505.9 [M+H]⁺. HPLC Purity (254 nm): 95.77%; t_{R}=9.105 min.

AC2020601-0295

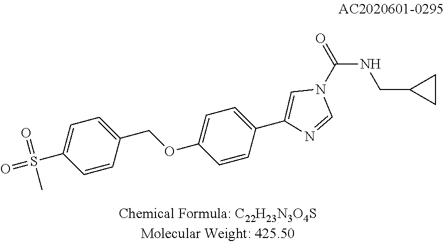 [SMILES: CS(=O)(=O)c1ccc(COc2ccc(-c3cn(C(=O)NCC4CC4)cn3)cc2)cc1]

### The Synthesis of tert-butyl 4-(4-((4-(methylsulfonyl)benzyl)oxy)phenyl)-1H-imidazole-1-carboxylate (243-2)

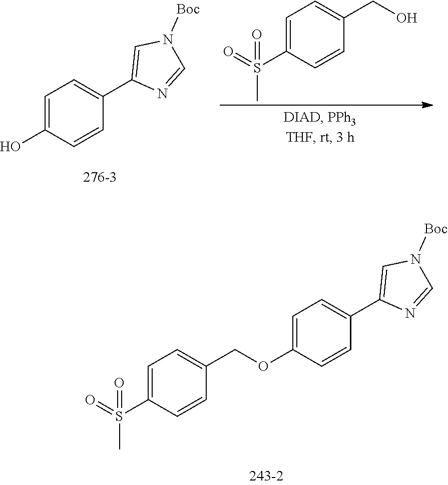 [SMILES: CC(C)(C)OC(=O)n1cnc(-c2ccc(O)cc2)c1 ; CS(=O)(=O)c1ccc(CO)cc1 ; CC(C)(C)OC(=O)n1cnc(-c2ccc(OCc3ccc(S(C)(=O)=O)cc3)cc2)c1]

To a suspension of 276-3 (780 mg, 3.0 mmol) in THF (10 mL) was added (4-(methylsulfonyl)phenyl)methanol (558 mg, 3.0 mmol), PPh₃ (942 mg, 3.6 mmol) and DIAD (789 mg, 3.9 mmol). The reaction mixture was stirred at room temperature for 3 hours until the reaction was complete (by LCMS). Saturated NaCl solution was added and the reaction mixture was extracted with EtOAc (20 mL), the combined organic layer was washed with brine, dried over anhydrous Na₂SO₄ and evaporated under reduced pressure. The crude was purified by silica gel chromatography (10% MeOH/DCM) to afford 243-2 (680 mg, yield: 53%) as yellow oil.

### The Synthesis of 4-(4-((4-(methylsulfonyl)benzyl)oxy)phenyl)-1H-imidazole (243-3)

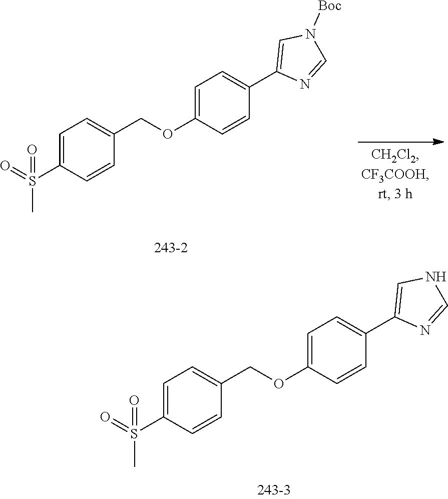 [SMILES: CC(C)(C)OC(=O)n1cnc(-c2ccc(OCc3ccc(S(C)(=O)=O)cc3)cc2)c1 ; CS(=O)(=O)c1ccc(COc2ccc(-c3c[nH]cn3)cc2)cc1]

To a stirred solution of 243-2 (400 mg, 0.93 mmol) in CH₂Cl₂ (10 mL) was added TFA (5 mL). The reaction mixture was stirred at room temperature for 3 hours until the reaction was complete (by LCMS). The mixture was concentrated under reduced pressure to afford 243-3 (200 mg, yield: 65%) as yellow oil, which was used to the next step without purification.

### The Synthesis of N-(cyclopropylmethyl)-4-(4-((4-(methylsulfonyl)benzyl)oxy)phenyl)-1H-imidazole-1-carboxamide (AC2020601-0295)

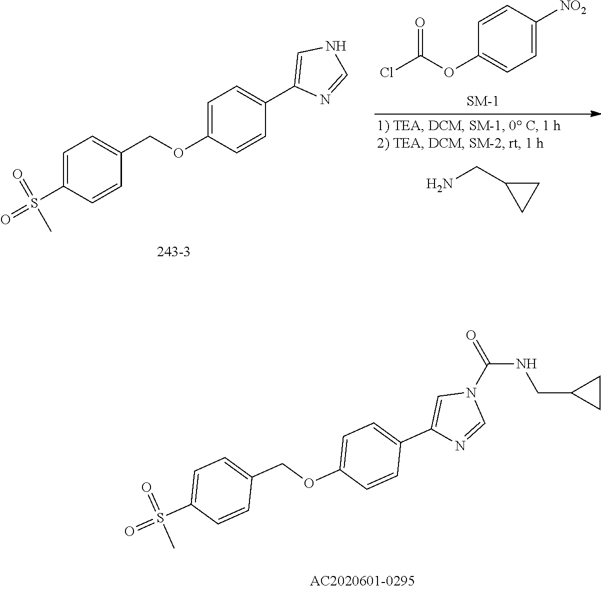

To a solution of 243-3 (300 mg, 0.91 mmol) in DCM (5 mL) was added 4-nitrophenyl carbonochloridate (221 mg, 1.10 mmol) and Et₃N (5 mL) at 0° C. and stirred for 1 hour. Then the reaction mixture was allowed to warm to room temperature and cyclopropylmethanamine (78 mg, 1.10 mmol) was added. After 1 hour stirring, the reaction mixture was quenched with water, extracted with DCM (20 mL), the combined organic layer was washed with brine, dried over anhydrous Na₂SO₄ and evaporated under reduced pressure. The crude was purified by silica gel chromatography (5% MeOH/DCM) to afford AC2020601-0295 (76 mg, yield: 20%) as a white solid. ¹H NMR (400 MHz, CDCl₃) δ 8.96 (br, 1H), 7.98 (d, J=8.0 Hz, 2H), 7.76 (d, J=8.8 Hz, 2H), 7.64-7.67 (m, 3H), 7.26 (s, 1H), 7.02 (d, J=8.8 Hz, 2H), 5.21 (s, 2H), 3.32-3.35 (m, 2H), 3.07 (s, 3H), 1.12-1.19 (m, 1H), 0.59-0.64 (m, 2H), 0.33-0.37 (m, 2H). LC-MS m/z: 425.9 [M+H]⁺. HPLC Purity (254 nm): 95.16%; t_{R}=7.160 min.

AC2020601-0296

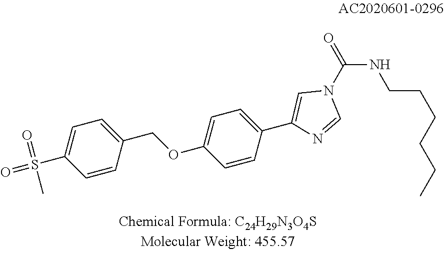 [SMILES: CCCCCCNC(=O)n1cnc(-c2ccc(OCc3ccc(S(C)(=O)=O)cc3)cc2)c1]

### The Synthesis of tert-butyl 4-(4-((4-(methylsulfonyl)benzyl)oxy)phenyl)-1H-imidazole-1-carboxylate (243-2)

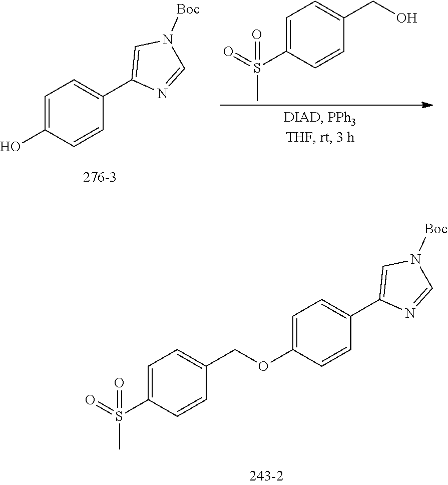 [SMILES: CC(C)(C)OC(=O)n1cnc(-c2ccc(O)cc2)c1 ; CS(=O)(=O)c1ccc(CO)cc1 ; CC(C)(C)OC(=O)n1cnc(-c2ccc(OCc3ccc(S(C)(=O)=O)cc3)cc2)c1]

To a suspension of 276-3 (780 mg, 3.0 mmol) in THF (10 mL) was added (4-(methylsulfonyl)phenyl)methanol (558 mg, 3.0 mmol), PPh₃ (942 mg, 3.6 mmol) and DIAD (789 mg, 3.9 mmol). The reaction mixture was stirred at room temperature for 3 hours until the reaction was complete (by LCMS). Saturated NaCl solution was added and the reaction mixture was extracted with EtOAc (20 mL), the combined organic layer was washed with brine, dried over anhydrous Na₂SO₄ and evaporated under reduced pressure. The crude was purified by silica gel chromatography (10% MeOH/DCM) to afford 243-2 (680 mg, yield: 53%) as yellow oil.

### The Synthesis of 4-(4-((4-(methylsulfonyl)benzyl)oxy)phenyl)-1H-imidazole (243-3)

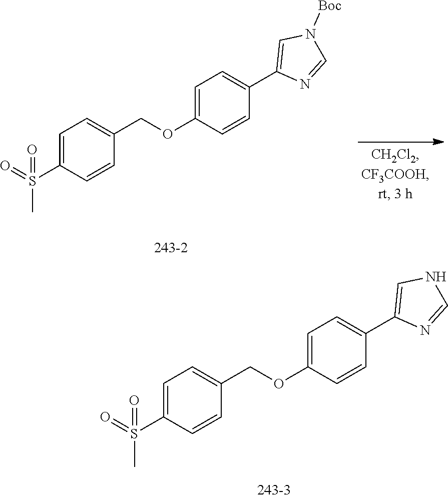 [SMILES: CC(C)(C)OC(=O)n1cnc(-c2ccc(OCc3ccc(S(C)(=O)=O)cc3)cc2)c1 ; CS(=O)(=O)c1ccc(COc2ccc(-c3c[nH]cn3)cc2)cc1]

To a stirred solution of 243-2 (400 mg, 0.93 mmol) in CH₂Cl₂ (10 mL) was added TFA (5 mL). The reaction mixture was stirred at room temperature for 3 hours until the reaction was complete (by LCMS). The mixture was concentrated under reduced pressure to afford 243-3 (200 mg, yield: 65%) as yellow oil, which was used to the next step without purification.

### The Synthesis of N-hexyl-4-(4-((4-(methylsulfonyl)benzyl)oxy)phenyl)-1H-imidazole-1-carboxamide (AC2020601-0296)

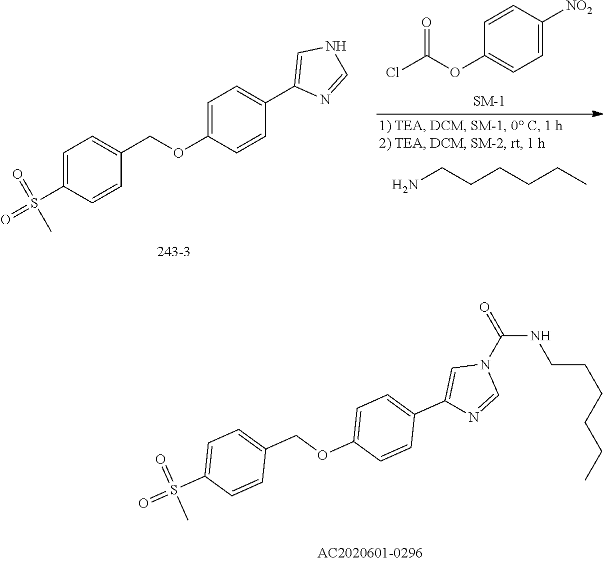

To a solution of 243-3 (300 mg, 0.91 mmol) in DCM (5 mL) was added 4-nitrophenyl carbonochloridate (221 mg, 1.10 mmol) and Et₃N (5 mL) at 0° C. and stirred for 1 hour. Then the reaction mixture was allowed to warm to room temperature and hexan-1-amine (111 mg, 1.10 mmol) was added. After 1 hour stirring, the reaction mixture was quenched with water, extracted with DCM (20 mL), the combined organic layer was washed with brine, dried over anhydrous Na₂SO₄ and evaporated under reduced pressure. The crude was purified by silica gel chromatography (5% MeOH/DCM) to afford AC2020601-0296 (29 mg, yield: 7%) as a white solid. ¹H NMR (400 MHz, CDCl₃) δ 8.41 (s, 1H), 7.97 (d, J=8.0 Hz, 2H), 7.71 (d, J=8.8 Hz, 2H), 7.64 (d, J=8.4 Hz, 2H), 7.54 (d, J=1.2 Hz, 1H), 6.99 (d, J=8.8 Hz, 2H), 6.20 (s, 1H), 5.18 (s, 2H), 3.42-3.47 (m, 2H), 3.07 (s, 3H), 1.63-1.68 (m, 2H), 1.31-1.41 (m, 6H), 0.90 (t, J=7.2 Hz, 2H). LC-MS m/z: 456.1 [M+H]⁺. HPLC Purity (254 nm): 95.48%; t_{R}=8.627 min.

AC2020601-0297

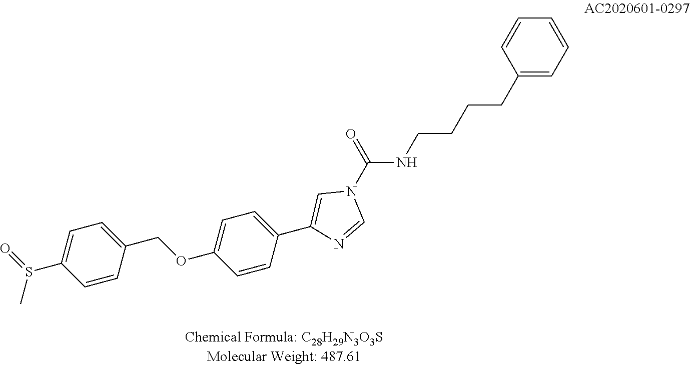 [SMILES: CS(=O)c1ccc(COc2ccc(-c3cn(C(=O)NCCCCc4ccccc4)cn3)cc2)cc1]

### The Synthesis of (4-(methylsulfinyl)phenyl)methanol (297-1)

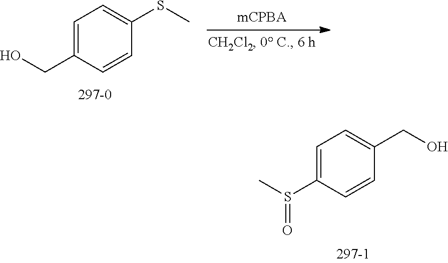

To a suspension of 297-0 (300 mg, 1.95 mmol) in CH₂Cl₂ (10 mL) was added m-CPBA (336 mg, 1.95 mmol) at 0° C. The reaction mixture was stirred at room temperature for 6 hours until the reaction was complete (by LCMS). Saturated Na₂SO₃ solution was added and the reaction mixture was extracted with CH₂Cl₂ (20 mL), the combined organic layer was washed with brine, dried over anhydrous Na₂SO₄ and evaporated under reduced pressure. The crude was purified by silica gel chromatography (26% EA/PE) to afford 297-1 (230 mg, yield: 69%) as yellow oil.

### The Synthesis of tert-butyl 4-(4-((4-(methylsulfinyl)benzyl)oxy)phenyl)-1H-imidazole-1-carboxylate (297-2)

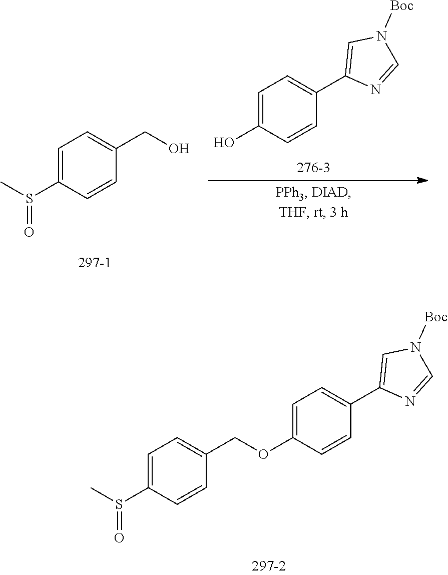 [SMILES: CS(=O)c1ccc(CO)cc1 ; CC(C)(C)OC(=O)n1cnc(-c2ccc(O)cc2)c1 ; CS(=O)c1ccc(COc2ccc(-c3cn(C(=O)OC(C)(C)C)cn3)cc2)cc1]

To a suspension of 276-3 (780 mg, 3.00 mmol) in THF (10 mL) was added (4-(methylsulfinyl)phenyl)methanol (510 mg, 3.00 mmol), PPh₃ (942 mg, 3.60 mmol) and DIAD (789 mg, 3.90 mmol). The reaction mixture was stirred at room temperature for 3 hours until the reaction was complete (by LCMS). Saturated NaCl solution was added and the reaction mixture was extracted with EtOAc (20 mL), the combined organic layer was washed with brine, dried over anhydrous Na₂SO₄ and evaporated under reduced pressure. The crude was purified by silica gel chromatography (4% MeOH/DCM) to afford 297-2 (1.00 g, yield: 81%) as yellow oil.

### The Synthesis of 4-(4-((4-(methylthio)benzyl)oxy)phenyl)-1H-imidazole (297-3)

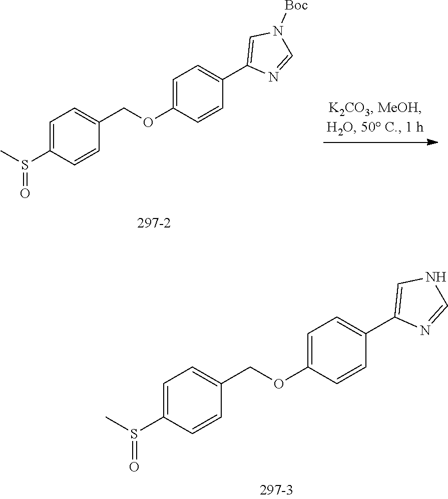 [SMILES: CS(=O)c1ccc(COc2ccc(-c3cn(C(=O)OC(C)(C)C)cn3)cc2)cc1 ; CS(=O)c1ccc(COc2ccc(-c3c[nH]cn3)cc2)cc1]

To a suspension of 297-2 (314 mg, 0.76 mmol) in MeOH (5 mL) and H₂O (1 mL) was added K₂CO₃ (209 mg, 1.51 mmol). The reaction mixture was stirred at 50° C. for 1 hour until the reaction was complete (by LCMS). Saturated NaCl solution was added and the reaction mixture was extracted with EtOAc (50 mL), the combined organic layer was washed with brine, dried over anhydrous Na₂SO₄ and evaporated under reduced pressure to afford 297-3 (200 mg, yield: 84%) as a yellow solid.

### The Synthesis of 4-(4-((4-(methylsulfinyl)benzyl)oxy)phenyl)-N-(4-phenylbutyl)-1H-imidazole-1-carboxamide (AC2020601-0297)

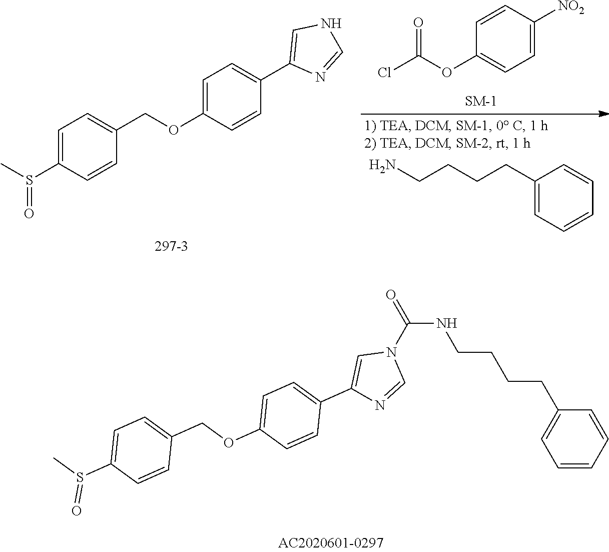 [SMILES: CS(=O)c1ccc(COc2ccc(-c3c[nH]cn3)cc2)cc1 ; O=C(Cl)Oc1ccc([N+](=O)[O-])cc1 ; NCCCCc1ccccc1 ; CS(=O)c1ccc(COc2ccc(-c3cn(C(=O)NCCCCc4ccccc4)cn3)cc2)cc1]

To a solution of 297-3 (110 mg, 0.35 mmol) in DCM (5 mL) was added 4-nitrophenyl carbonochloridate (85 mg, 0.42 mmol) and Et₃N (5 mL) at 0° C. The reaction mixture was stirred for 1 hour 0° C. Then the reaction mixture was allowed to warm to room temperature and 4-phenylbutan-1-amine (63 mg, 0.42 mmol) was added. After 1 hour stirring, the reaction mixture was quenched with water, extracted with DCM (20 mL), the combined organic layer was washed with brine, dried over anhydrous Na₂SO₄ and evaporated under reduced pressure. The crude was purified by silica gel chromatography (5% MeOH/DCM) to afford AC2020601-0297 (7 mg, yield: 5%) as a white solid. ¹H NMR (400 MHz, CDCl₃) δ 8.39 (s, 1H), 7.66-7.68 (m, 4H), 7.59 (d, J=8.0 Hz, 2H), 7.55 (d, J=0.8 Hz, 1H), 7.26-7.32 (m, 2H), 7.18-7.21 (m, 3H), 6.98 (d, J=9.2 Hz, 2H), 6.48 (s, 1H), 5.13 (s, 2H), 3.44-3.48 (m, 2H), 2.72 (s, 3H), 2.68 (t, J=7.2 Hz, 2H), 1.71-1.74 (m, 4H). LC-MS m/z: 488.0 [M+H]⁺. HPLC Purity (254 nm): 99.99%; t_{R}=9.105 min.

AC2020601-0301

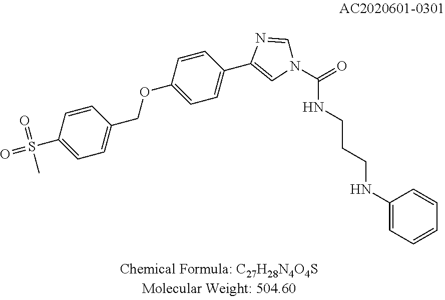 [SMILES: CS(=O)(=O)c1ccc(COc2ccc(-c3cn(C(=O)NCCCNc4ccccc4)cn3)cc2)cc1]

### The Synthesis of N¹-phenylpropane-1,3-diamine (301-2)

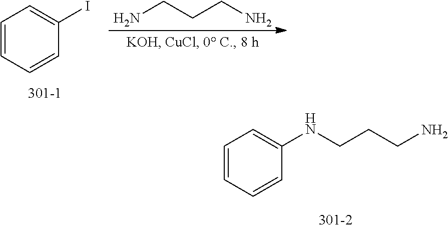 [SMILES: Ic1ccccc1 ; NCCCN ; NCCCNc1ccccc1]

To a suspension of 301-1 (2.0 g, 10.0 mmol) in propane-1,3-diamine (2.2 g, 30.0 mmol) was added KOH (1.1 g, 20.0 mmol) and CuCl (0.1 g, 1.0 mmol). The reaction mixture was stirred at 0° C. for 8 hours until the reaction was complete (by LCMS). Then the reaction mixture was poured into water (20 mL), extracted with EtOAc (20 mL). The combined organic layer was washed with water and brine, dried over anhydrous Na₂SO₄, filtered and evaporated under reduced pressure. The crude was purified by silica gel chromatography (5-10% MeOH in CH₂Cl₂) to get 301-2 (1.6 g, yield: 87%) as green oil.

### The Synthesis of 4-(4-(4-(methylsulfonyl)benzyloxy)phenyl)-N-(3-(phenylamino)propyl)-1H-imidazole-1-carboxamide (AC2020601-0301)

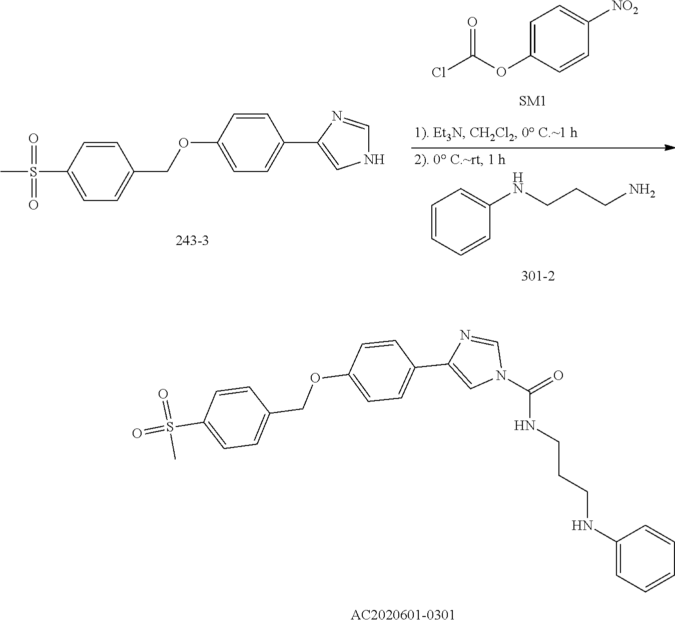 [SMILES: O=C(Cl)Oc1ccc([N+](=O)[O-])cc1 ; CS(=O)(=O)c1ccc(COc2ccc(-c3c[nH]cn3)cc2)cc1 ; NCCCNc1ccccc1 ; CS(=O)(=O)c1ccc(COc2ccc(-c3cn(C(=O)NCCCNc4ccccc4)cn3)cc2)cc1]

To a solution of 243-3 (328.0 mg, 1.0 mmol) in DCM (10 mL) was added SM1 (302.0 mg, 1.5 mmol) and Et₃N (10 mL) at 0° C. and stirred for 1 hour. Then the reaction mixture was allowed to warm to rt and 301-2 (301.0 mg, 2.0 mmol) was added. After 1 hour stirring, the reaction mixture was quenched with water, extracted with DCM (20 mL), the combined organic layer was washed with brine, dried over anhydrous Na₂SO₄ and evaporated under reduced pressure. The crude was purified by silica gel chromatography (10% MeOH/DCM) to afford AC2020601-0301 (33.0 mg, yield: 15.2%) as a white solid. ¹H NMR (400 MHz, DMSO-d₆) δ 8.52 (t, J=5.2 Hz, 1H), 8.27 (d, J=0.8 Hz, 1H), 8.02 (s, 1H), 7.96 (d, J=8.0 Hz, 2H), 7.71-7.75 (m, 4H), 7.04-7.08 (m, 4H), 6.57 (d, J=7.6 Hz, 2H), 6.51 (t, J=7.6 Hz, 1H), 5.58 (t, J=5.6 Hz, 1H), 5.28 (s, 2H), 3.35-3.39 (m, 2H), 3.22 (s, 3H), 3.06-3.11 (m, 2H), 1.82-1.86 (m, 2H). LC-MS m/z: N/A. HPLC Purity (254 nm): 94.16%; t_{R}=8.845 min.

AC2020601-0303

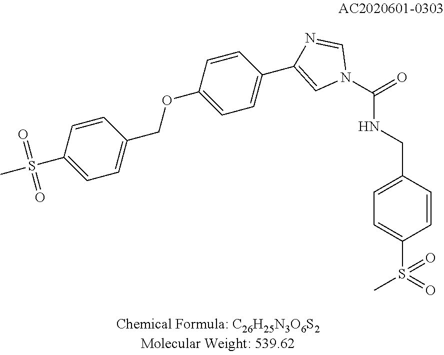 [SMILES: CS(=O)(=O)c1ccc(CNC(=O)n2cnc(-c3ccc(OCc4ccc(S(C)(=O)=O)cc4)cc3)c2)cc1]

### The Synthesis of tert-butyl 4-(4-((4-(methylsulfonyl)benzyl)oxy)phenyl)-1H-imidazole-1-carboxylate (243-2)

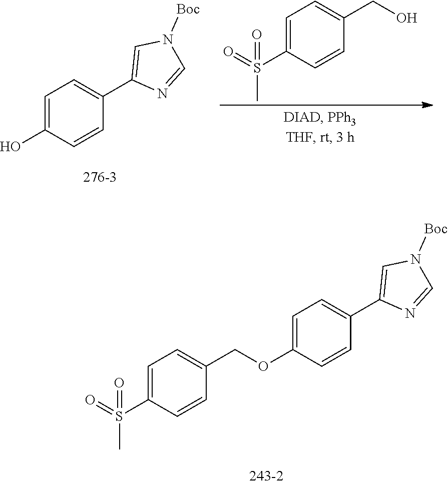 [SMILES: CC(C)(C)OC(=O)n1cnc(-c2ccc(O)cc2)c1 ; CS(=O)(=O)c1ccc(CO)cc1 ; CC(C)(C)OC(=O)n1cnc(-c2ccc(OCc3ccc(S(C)(=O)=O)cc3)cc2)c1]

To a suspension of 276-3 (780 mg, 3.0 mmol) in THF (10 mL) was added (4-(methylsulfonyl)phenyl)methanol (558 mg, 3.0 mmol), PPh₃ (942 mg, 3.6 mmol) and DIAD (789 mg, 3.9 mmol). The reaction mixture was stirred at room temperature for 3 hours until the reaction was complete (by LCMS). Saturated NaCl solution was added and the reaction mixture was extracted with EtOAc (20 mL), the combined organic layer was washed with brine, dried over anhydrous Na₂SO₄ and evaporated under reduced pressure. The crude was purified by silica gel chromatography (10% MeOH/DCM) to afford 243-2 (680 mg, yield: 53%) as yellow oil.

### The Synthesis of 4-(4-((4-(methylsulfonyl)benzyl)oxy)phenyl)-1H-imidazole (243-3)

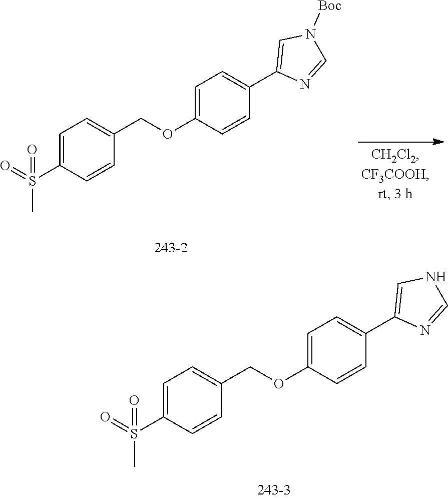 [SMILES: CC(C)(C)OC(=O)n1cnc(-c2ccc(OCc3ccc(S(C)(=O)=O)cc3)cc2)c1 ; CS(=O)(=O)c1ccc(COc2ccc(-c3c[nH]cn3)cc2)cc1]

To a stirred solution of 243-2 (400 mg, 0.93 mmol) in CH₂Cl₂ (10 mL) was added TFA (5 mL). The reaction mixture was stirred at room temperature for 3 hours until the reaction was complete (by LCMS). The mixture was concentrated under reduced pressure to afford 243-3 (200 mg, yield: 65%) as yellow oil, which was used to the next step without purification.

### The Synthesis of N-(4-(methylsulfonyl)benzyl)-4-(4-((4-(methylsulfonyl)benzyl)oxy)phenyl)-1H-imidazole-1-carboxamide (AC2020601-0303)

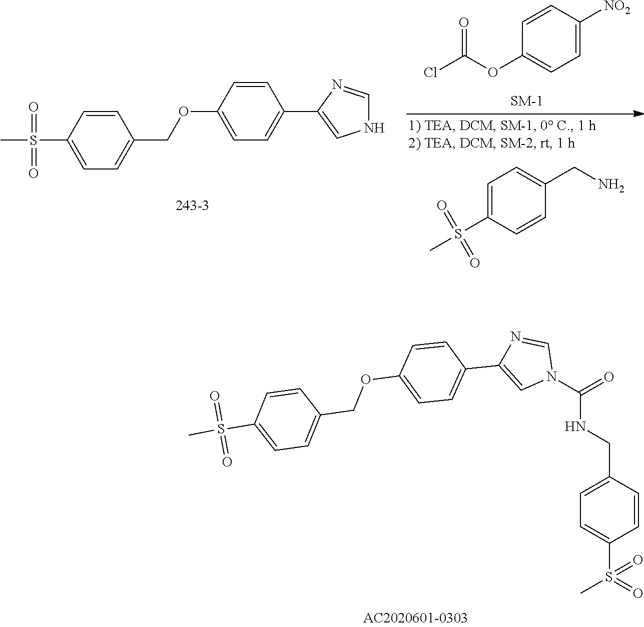 [SMILES: O=C(Cl)Oc1ccc([N+](=O)[O-])cc1 ; CS(=O)(=O)c1ccc(COc2ccc(-c3c[nH]cn3)cc2)cc1 ; CS(=O)(=O)c1ccc(CN)cc1 ; CS(=O)(=O)c1ccc(CNC(=O)n2cnc(-c3ccc(OCc4ccc(S(C)(=O)=O)cc4)cc3)c2)cc1]

To a solution of 243-3 (200 mg, 0.61 mmol) in DCM (5 mL) was added 4-nitrophenyl carbonochloridate (148 mg, 0.73 mmol) and Et₃N (5 mL) at 0° C. and stirred for 1 hour. Then the reaction mixture was allowed to warm to room temperature and (4-(methylsulfonyl)phenyl)methanamine (135 mg, 0.73 mmol) was added. After 1 hour stirring, the reaction mixture was quenched with water, extracted with DCM (20 mL), the combined organic layer was washed with brine, dried over anhydrous Na₂SO₄ and evaporated under reduced pressure. The crude was purified by silica gel chromatography (5% MeOH/DCM) to afford AC2020601-0303 (65 mg, yield: 20%) as a white solid. ¹H NMR (400 MHz, DMSO-d₆) δ 9.22 (t, J=5.6 Hz, 1H), 8.33 (d, J=1.2 Hz, 1H), 8.06 (d, J=1.2 Hz, 1H), 7.91-7.97 (m, 4H), 7.72-7.76 (m, 4H), 7.64 (d, J=8.4 Hz, 2H), 7.07 (d, J=8.8 Hz, 2H), 5.28 (s, 2H), 4.59 (d, J=5.6 Hz, 2H), 3.23 (s, 3H), 3.21 (s, 3H). LC-MS m/z: 539.8 [M+H]⁺. HPLC Purity (254 nm): 96.43%; t_{R}=7.697 min.

AC2020601-0304

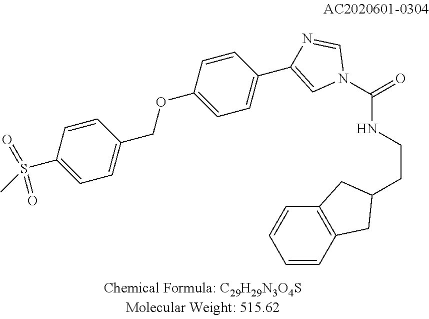 [SMILES: CS(=O)(=O)c1ccc(COc2ccc(-c3cn(C(=O)NCCC4Cc5ccccc5C4)cn3)cc2)cc1]

### The Synthesis of 2-(2,3-dihydro-1H-inden-2-yl)acetamide (304-1-2)

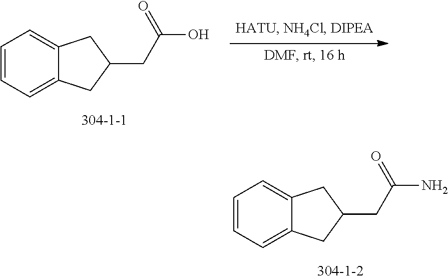 [SMILES: O=C(O)CC1Cc2ccccc2C1 ; NC(=O)CC1Cc2ccccc2C1]

To a suspension of 304-1-1 (1.76 g, 10.0 mmol) in DMF (30 mL) was added HATU (7.6 g, 20.0 mmol), DIPEA (7.75 g, 60 mmol) and NH₄Cl (1.6 g, 30.0 mmol). The reaction mixture was stirred at room temperature for 16 hours until the reaction was complete (by LCMS). Saturated NaCl solution was added and the reaction mixture was extracted with EtOAc (20 mL), the combined organic layer was washed with brine, dried over anhydrous Na₂SO₄ and evaporated under reduced pressure. The crude was purified by silica gel chromatography (10% MeOH/DCM) to afford 304-1-2 (500 mg, yield: 29%) as yellow oil.

### The Synthesis of 2-(2,3-dihydro-1H-inden-2-yl)ethanamine (304-1-3)

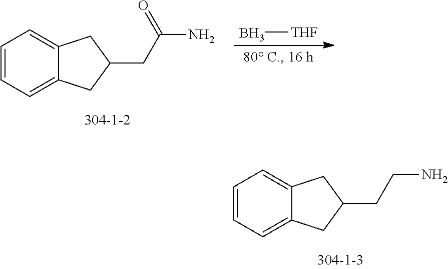

To a stirred solution of 304-1-2 (500 mg, 2.85 mmol) in BH₃-THF (10 mL), the reaction mixture was allowed to warm to 80° C. for 16 hours until the reaction was complete (by LCMS). The mixture was concentrated under reduced pressure to afford 304-1-3 (300 mg, yield: 65%) as yellow oil, which was used to the next step without purification.

### The Synthesis of N-(2-(2,3-dihydro-1H-inden-2-yl)ethyl)-4-(4-(4-(methylsulfonyl)benzyloxy)phenyl)-1H-imidazole-1-carboxamide (AC2020601-0304)

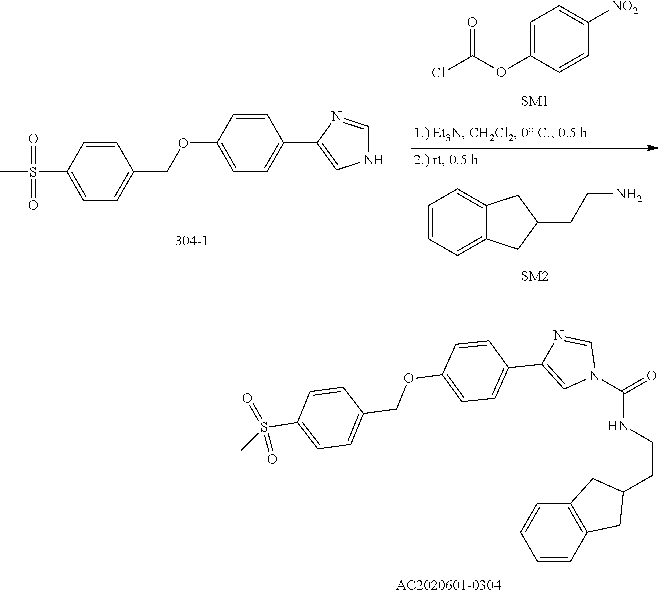 [SMILES: O=C(Cl)Oc1ccc([N+](=O)[O-])cc1 ; CS(=O)(=O)c1ccc(COc2ccc(-c3c[nH]cn3)cc2)cc1 ; NCCC1Cc2ccccc2C1 ; CS(=O)(=O)c1ccc(COc2ccc(-c3cn(C(=O)NCCC4Cc5ccccc5C4)cn3)cc2)cc1]

To a solution of 304-1 (200 mg, 0.61 mmol) in DCM (10 mL) was added SM1 (184.0 mg, 0.92 mmol) and Et₃N (10 mL) at 0° C. and stirred for 0.5 hour. Then the reaction mixture was allowed to warm to room temperature and SM2 (197.0 mg, 1.22 mmol) was added. After 0.5 hour stirring, the reaction mixture was quenched with water, extracted with DCM (20 mL), the combined organic layer was washed with brine, dried over anhydrous Na₂SO₄ and evaporated under reduced pressure. The crude was purified by silica gel chromatography (10% MeOH/DCM) to afford AC2020601-0304 (15.08 mg, yield: 4.8%) as a white solid. ¹H NMR (400 MHz, CDCl₃) δ 8.52 (t, J=5.6 Hz, 1H), 8.27 (d, J=1.2 Hz, 1H), 8.01 (d, J=1.2 Hz, 1H), 7.96 (d, J=8.0 Hz, 2H), 7.71-7.75 (m, 4H), 7.19-7.21 (m, 2H), 7.05-7.11 (m, 4H), 5.28 (s, 2H), 3.35-3.39 (m, 2H), 3.22 (s, 3H), 3.03-3.09 (m, 2H), 2.56-2.62 (m, 3H), 3.06 (q, J=6.8 Hz, 2H). LC-MS m/z: 515.9 [M+H]⁺. HPLC Purity (254 nm): 96.61%; t_{R}=9.681 min.

AC2020601-0306

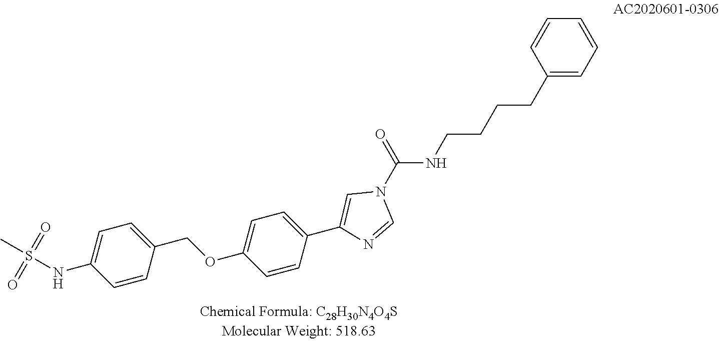 [SMILES: CS(=O)(=O)Nc1ccc(COc2ccc(-c3cn(C(=O)NCCCCc4ccccc4)cn3)cc2)cc1]

### The Synthesis of tert-butyl 4-(4-((4-nitrobenzyl)oxy)phenyl)-1H-imidazole-1-carboxylate (306-2)

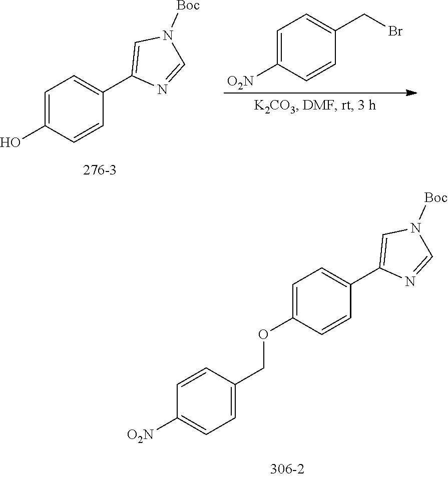 [SMILES: CC(C)(C)OC(=O)n1cnc(-c2ccc(O)cc2)c1 ; O=[N+]([O-])c1ccc(CBr)cc1 ; CC(C)(C)OC(=O)n1cnc(-c2ccc(OCc3ccc([N+](=O)[O-])cc3)cc2)c1]

To a suspension of 276-3 (5.2 g, 19.98 mmol) in DMF (10 mL) was added K₂CO₃ (5.5 g, 39.96 mmol) and 1-(bromomethyl)-4-nitrobenzene (8.6 g, 39.96 mmol). The reaction mixture was stirred at room temperature for 3 hours until the reaction was complete (by LCMS). Saturated NaCl solution was added and the reaction mixture was extracted with EtOAc (50 mL), the combined organic layer was washed with brine, dried over anhydrous Na₂SO₄ and evaporated under reduced pressure. The crude was purified by silica gel chromatography (5% MeOH/DCM) to afford 306-2 (5.0 g, yield: 6₃%) as a yellow solid.

### The Synthesis of tert-butyl 4-(4-((4-aminobenzyl)oxy)phenyl)-1H-imidazole-1-carboxylate (306-3)

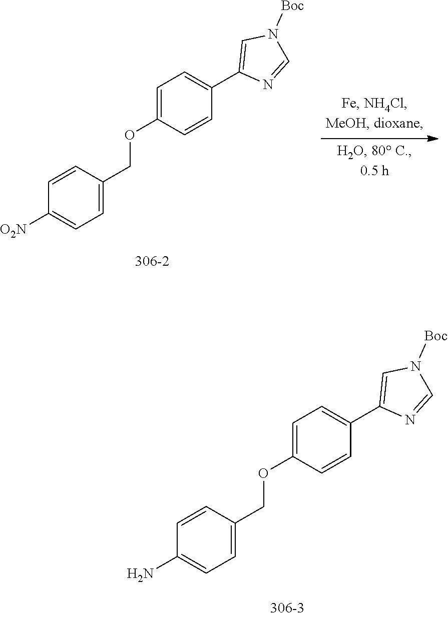 [SMILES: CC(C)(C)OC(=O)n1cnc(-c2ccc(OCc3ccc([N+](=O)[O-])cc3)cc2)c1 ; CC(C)(C)OC(=O)n1cnc(-c2ccc(OCc3ccc(N)cc3)cc2)c1]

To a suspension of 306-2 (3.80 g, 9.61 mmol) in MeOH (7 mL), dioxane (2 mL) and H₂O (1 mL), was added Fe (5.37 g, 96.10 mmol) and NH₄Cl (5.14 g, 96.10 mmol). The reaction mixture was stirred at 80° C. for 0.5 hours until the reaction was complete (by LCMS). Saturated NaCl solution was added and the reaction mixture was extracted with EtOAc (50 mL), the combined organic layer was washed with brine, dried over anhydrous Na₂SO₄ and evaporated under reduced pressure. The crude was purified by silica gel chromatography (5% MeOH/DCM) to afford 306-3 (0.5 g, yield: 14%) as a yellow solid.

### The Synthesis of tert-butyl 4-(4-((4-(methylsulfonamido)benzyl)oxy)phenyl)-1H-imidazole-1-carboxylate (306-4)

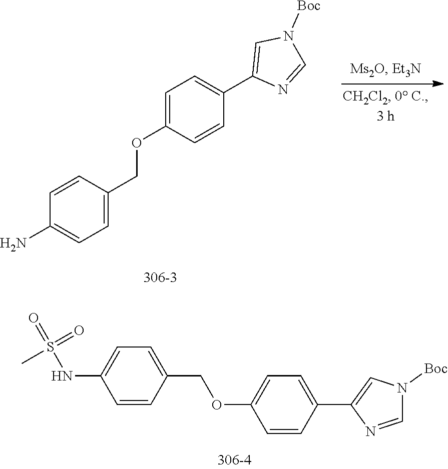 [SMILES: CC(C)(C)OC(=O)n1cnc(-c2ccc(OCc3ccc(N)cc3)cc2)c1 ; CC(C)(C)OC(=O)n1cnc(-c2ccc(OCc3ccc(NS(C)(=O)=O)cc3)cc2)c1]

To a suspension of 306-3 (500 mg, 1.37 mmol) in CH₂Cl₂ (10 mL) was added Et₃N (277 mg, 2.74 mmol) and Ms₂O (358 mg, 2.05 mmol) at 0° C. The reaction mixture was stirred at room temperature for 3 hours until the reaction was complete (by LCMS). Water was added and the reaction mixture was extracted with EtOAc (50 mL), the combined organic layer was washed with brine, dried over anhydrous Na₂SO₄ and evaporated under reduced pressure. The crude was purified by silica gel chromatography (5% MeOH/DCM) to afford 306-4 (0.4 g, yield: 66%) as a yellow solid.

### The Synthesis of N-(4-((4-(1H-imidazol-4-yl)phenoxy)methyl)phenyl)methanesulfonamide (306-5)

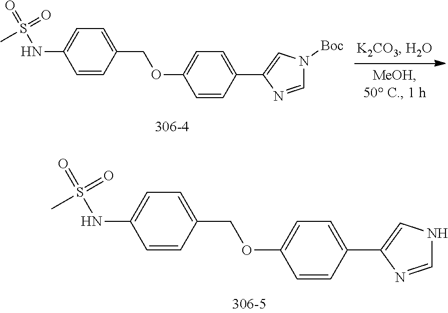 [SMILES: CC(C)(C)OC(=O)n1cnc(-c2ccc(OCc3ccc(NS(C)(=O)=O)cc3)cc2)c1 ; CS(=O)(=O)Nc1ccc(COc2ccc(-c3c[nH]cn3)cc2)cc1]

To a stirred solution of 306-4 (300 mg, 0.68 mmol) in MeOH (10 mL) and H₂O (1 mL) was added K₂CO₃ (187 mg, 1.35 mmol). The reaction mixture was stirred at 50° C. for 1 hour until the reaction was complete (by LCMS). The reaction mixture was quenched with water, extracted with EA (20 mL), the combined organic layer was washed with brine, dried over anhydrous Na₂SO₄. The mixture was concentrated under reduced pressure to afford 306-5 (200 mg, yield: 86%) as a white solid, which was used to the next step without purification.

### The Synthesis of 4-(4-((4-(methylsulfonamido)benzyl)oxy)phenyl)-N-(4-phenylbutyl)-1H-imidazole-1-carboxamide (AC2020601-0306)

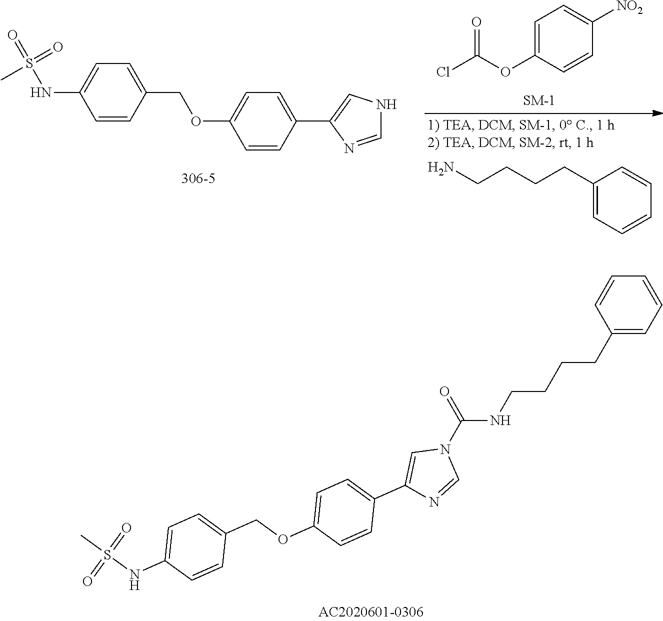

To a solution of 306-5 (100 mg, 0.29 mmol) in DCM (5 mL) was added 4-nitrophenyl carbonochloridate (70 mg, 0.35 mmol) and Et₃N (5 mL) at 0° C. and stirred for 1 hour. Then the reaction mixture was allowed to warm to room temperature and 4-phenylbutan-1-amine (52 mg, 0.35 mmol) was added. After 1 hour stirring, the reaction mixture was quenched with water, extracted with DCM (20 mL), the combined organic layer was washed with brine, dried over anhydrous Na₂SO₄ and evaporated under reduced pressure. The crude was purified by silica gel chromatography (5% MeOH/DCM) to afford AC2020601-0306 (8 mg, yield: 5%) as a white solid. ¹H NMR (400 MHz, DMSO-d₆) δ 9.80 (s, 1H), 8.47 (t, J=5.6 Hz, 1H), 8.25 (s, 1H), 7.99 (s, 1H), 7.71 (d, J=8.4 Hz, 2H), 7.43 (d, J=8.0 Hz, 2H), 7.15-7.30 (m, 7H), 7.03 (d, J=8.8 Hz, 2H), 5.06 (s, 2H), 3.26-3.31 (m, 2H), 2.99 (s, 3H), 2.62 (t, J=7.2 Hz, 2H), 1.55-1.65 (m, 4H). LC-MS m/z: 519.2 [M+H]⁺. HPLC Purity (254 nm): 94.56%; t_{R}=9.606 min.

AC2020601-0321

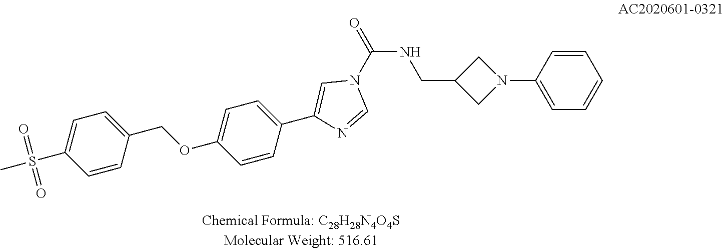 [SMILES: CS(=O)(=O)c1ccc(COc2ccc(-c3cn(C(=O)NCC4CN(c5ccccc5)C4)cn3)cc2)cc1]

### The Synthesis of tert-butyl ((1-phenylazetidin-3-yl)methyl)carbamate (321-2)

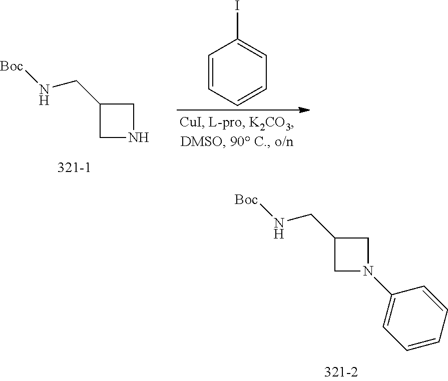 [SMILES: CC(C)(C)OC(=O)NCC1CNC1 ; Ic1ccccc1 ; CC(C)(C)OC(=O)NCC1CN(c2ccccc2)C1]

A mixture of 321-1 (2.00 g, 10.74 mmol), iodobenzene (4.38 g, 21.48 mmol), CuI (204 mg, 1.07 mmol), L-pro (124 mg, 1.07 mmol) and K₂CO₃ (2.97 g, 21.48 mmol) in DMSO (10 mL) was stirred at 90° C. overnight under N₂. The reaction mixture was quenched with water and extracted with EtOAc (50 mL), the combined organic layer was washed with brine, dried over anhydrous Na₂SO₄ and evaporated under reduced pressure. The crude was purified by silica gel chromatography (25% EA/PE) to afford 321-2 (2.10 g, yield: 75%) as yellow oil.

### The Synthesis of (1-phenylazetidin-3-yl)methanamine (321-3)

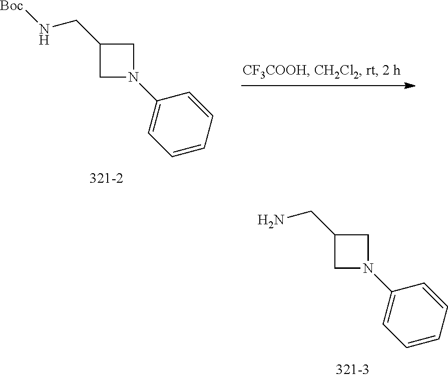 [SMILES: CC(C)(C)OC(=O)NCC1CN(c2ccccc2)C1 ; NCC1CN(c2ccccc2)C1]

To a stirred solution of 321-2 (2.00 g, 7.62 mmol) in CH₂Cl₂ (10 mL) was added TFA (5 mL). The reaction mixture was stirred at room temperature for 2 hours until the reaction was complete (by LCMS). The mixture was concentrated under reduced pressure to afford 321-3 (1.0 g, yield: 81%) as yellow oil, which was used to the next step without purification.

### The Synthesis of 4-(4-((4-(methylsulfonyl)benzyl)oxy)phenyl)-N-((1-phenylazetidin-3-yl)methyl)-1H-imidazole-1-carboxamide (AC2020601-0321)

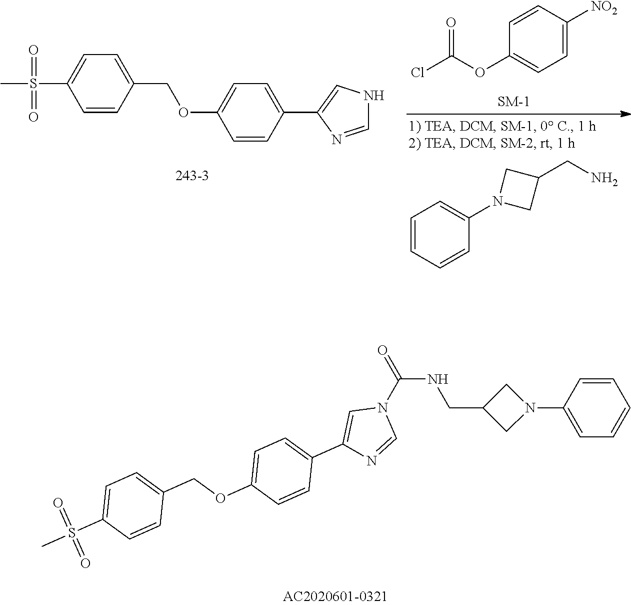 [SMILES: CS(=O)(=O)c1ccc(COc2ccc(-c3c[nH]cn3)cc2)cc1 ; O=C(Cl)Oc1ccc([N+](=O)[O-])cc1 ; NCC1CN(c2ccccc2)C1 ; CS(=O)(=O)c1ccc(COc2ccc(-c3cn(C(=O)NCC4CN(c5ccccc5)C4)cn3)cc2)cc1]

To a solution of 243-3 (100 mg, 0.30 mmol) in DCM (5 mL) was added 4-nitrophenyl carbonochloridate (74 mg, 0.37 mmol) and Et₃N (5 mL) at 0° C. and stirred for 1 hour. Then the reaction mixture was allowed to warm to room temperature and (1-phenylazetidin-3-yl)methanamine (59 mg, 0.37 mmol) was added. After 1 hour stirring, the reaction mixture was quenched with water, extracted with DCM (20 mL), the combined organic layer was washed with brine, dried over anhydrous Na₂SO₄ and evaporated under reduced pressure. The crude was purified by silica gel chromatography (5% MeOH/DCM) to afford AC2020601-0321 (46 mg, yield: 29%) as a white solid. ¹H NMR (400 MHz, DMSO-d₆) δ 8.68 (t, J=5.6 Hz, 1H), 8.28 (d, J=1.2 Hz, 1H), 8.01 (d, J=0.8 Hz, 1H), 7.96 (d, J=8.4 Hz, 2H), 7.72-7.75 (m, 4H), 7.14-7.18 (m, 2H), 7.07 (d, J=8.8 Hz, 2H), 6.64-6.68 (m, 1H), 6.41 (d, J=7.6 Hz, 2H), 5.28 (s, 2H), 3.87 (t, J=7.6 Hz, 2H), 3.55-3.60 (m, 4H), 3.22 (s, 3H), 2.91-2.95 (m, 1H). LC-MS m/z: 516.8 [M+H]⁺. HPLC Purity (254 nm): 96.10%; t_{R}=8.086 min.

AC2020601-0322

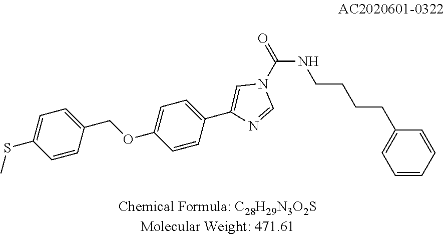 [SMILES: CSc1ccc(COc2ccc(-c3cn(C(=O)NCCCCc4ccccc4)cn3)cc2)cc1]

### The Synthesis of tert-butyl 4-(4-((4-(methylthio)benzyl)oxy)phenyl)-1H-imidazole-1-carboxylate (322-2)

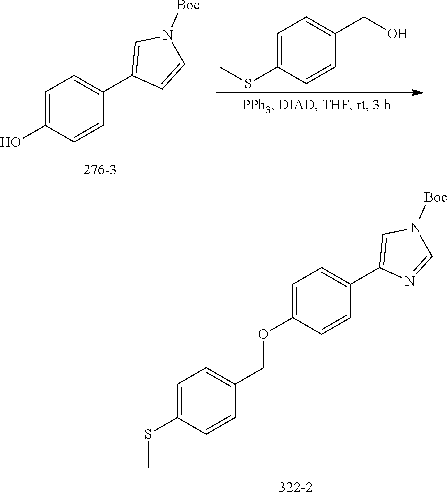

To a suspension of 276-3 (780 mg, 3.00 mmol) in THF (10 mL) was added (4-(methylthio)phenyl)methanol (462 mg, 3.00 mmol), PPh₃ (942 mg, 3.60 mmol) and DIAD (789 mg, 3.90 mmol). The reaction mixture was stirred at room temperature for 3 hours until the reaction was complete (by LCMS). Saturated NaCl solution was added and the reaction mixture was extracted with EtOAc (20 mL), the combined organic layer was washed with brine, dried over anhydrous Na₂SO₄ and evaporated under reduced pressure. The crude was purified by silica gel chromatography (4% MeOH/DCM) to afford 322-2 (1.00 g, yield: 84%) as yellow oil.

### The Synthesis of 4-(4-((4-(methylthio)benzyl)oxy)phenyl)-1H-imidazole (322-3)

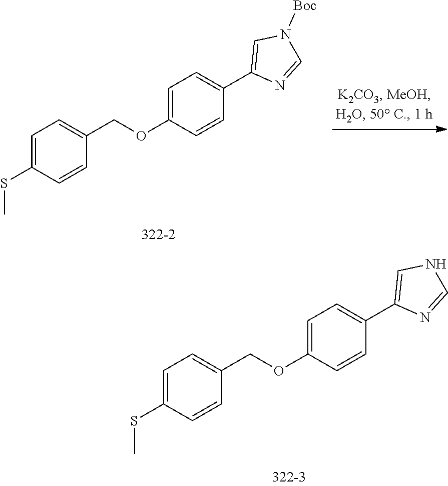 [SMILES: CSc1ccc(COc2ccc(-c3cn(C(=O)OC(C)(C)C)cn3)cc2)cc1 ; CSc1ccc(COc2ccc(-c3c[nH]cn3)cc2)cc1]

To a suspension of 322-2 (300 mg, 0.76 mmol) in MeOH (5 mL) and H₂O (1 mL) was added K₂CO₃ (209 mg, 1.51 mmol). The reaction mixture was stirred at 50° C. for 1 hour until the reaction was complete (by LCMS). Saturated NaCl solution was added and the reaction mixture was extracted with EtOAc (50 mL), the combined organic layer was washed with brine, dried over anhydrous Na₂SO₄ and evaporated under reduced pressure to afford 322-3 (210 mg, yield: 94%) as a yellow solid.

### The Synthesis of 4-(4-((4-(methylthio)benzyl)oxy)phenyl)-N-(4-phenylbutyl)-1H-imidazole-1-carboxamide (AC2020601-0322)

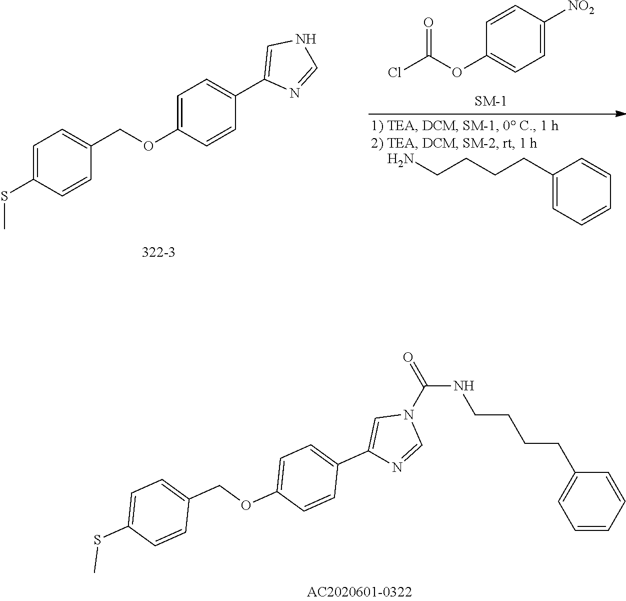 [SMILES: CSc1ccc(COc2ccc(-c3c[nH]cn3)cc2)cc1 ; O=C(Cl)Oc1ccc([N+](=O)[O-])cc1 ; NCCCCc1ccccc1 ; CSc1ccc(COc2ccc(-c3cn(C(=O)NCCCCc4ccccc4)cn3)cc2)cc1]

To a solution of 322-3 (210 mg, 0.71 mmol) in DCM (5 mL) was added 4-nitrophenyl carbonochloridate (171 mg, 0.85 mmol) and Et₃N (5 mL) at 0° C. and stirred for 1 hour. Then the reaction mixture was allowed to warm to room temperature and 4-phenylbutan-1-amine (127 mg, 0.85 mmol) was added. After 1 hour stirring, the reaction mixture was quenched with water, extracted with DCM (20 mL), the combined organic layer was washed with brine, dried over anhydrous Na₂SO₄ and evaporated under reduced pressure. The crude was purified by silica gel chromatography (5% MeOH/DCM) to afford AC2020601-0322 (100 mg, yield: 30%) as a white solid. ¹H NMR (400 MHz, CDCl₃) δ 8.19 (s, 1H), 7.69 (d, J=8.8 Hz, 2H), 7.44 (d, J=0.8 Hz, 1H), 7.36 (d, J=8.4 Hz, 2H), 7.26-7.31 (m, 4H), 7.18-7.22 (m, 3H), 6.99 (d, J=8.8 Hz, 2H), 5.74 (s, 1H), 5.04 (s, 2H), 3.44-3.49 (m, 2H), 2.68 (t, J=6.8 Hz, 2H), 2.49 (s, 3H), 1.66-1.76 (m, 7H). LC-MS m/z: 472.0 [M+H]⁺. HPLC Purity (254 nm): 96.99%; t_{R}=11.041 min.

AC2020601-0323

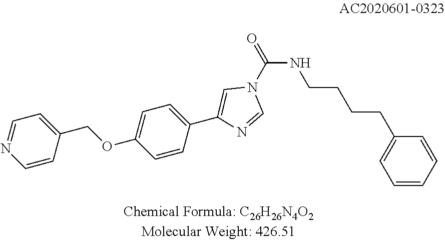 [SMILES: O=C(NCCCCc1ccccc1)n1cnc(-c2ccc(OCc3ccncc3)cc2)c1]

### The Synthesis of tert-butyl 4-(4-(pyridin-4-ylmethoxy)phenyl)-1H-imidazole-1-carboxylate(323-2)

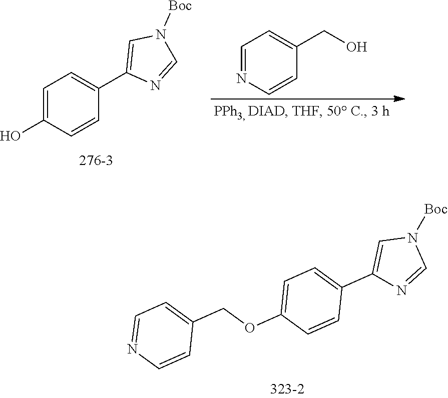 [SMILES: CC(C)(C)OC(=O)n1cnc(-c2ccc(O)cc2)c1 ; OCc1ccncc1 ; CC(C)(C)OC(=O)n1cnc(-c2ccc(OCc3ccncc3)cc2)c1]

To a suspension of 276-3 (520.0 mg, 2.0 mmol) in THF (10 mL) was added pyridin-4-ylmethanol (220.0 mg, 2.0 mmol), PPh₃ (628.0 mg, 2.4 mmol) and DIAD (526.0 mg, 2.6 mmol). The reaction mixture was heated to 50° C. for 3 hours until the reaction was complete (by LCMS). Saturated NaCl solution was added and the reaction mixture was extracted with EtOAc (20 mL), the combined organic layer was washed with brine, dried over anhydrous Na₂SO₄ and evaporated under reduced pressure. The crude was purified by silica gel chromatography (10% MeOH/DCM) to afford 323-2 (500 mg, yield: 71%) as yellow oil.

### The Synthesis of 4-((4-(1H-imidazol-4-yl)phenoxy)methyl)pyridine (323-3)

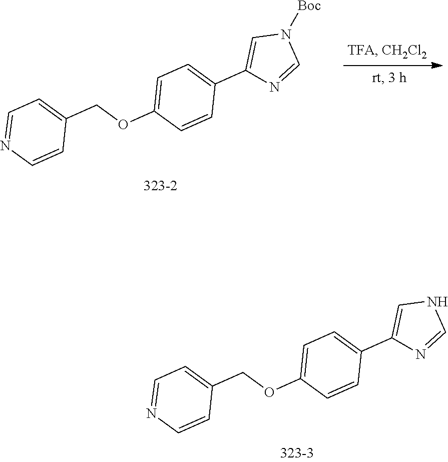 [SMILES: CC(C)(C)OC(=O)n1cnc(-c2ccc(OCc3ccncc3)cc2)c1 ; c1cc(COc2ccc(-c3c[nH]cn3)cc2)ccn1]

To a stirred solution of 323-2 (500 mg, 1.4 mmol) in CH₂Cl₂ (10 mL) was added TFA (5 mL). The reaction mixture was stirred at room temperature for 3 hours until the reaction was complete (by LCMS). The mixture was concentrated under reduced pressure to afford 323-3 (380 mg, yield: 100%) as yellow oil, which was used to the next step without purification.

### The Synthesis of N-(4-phenylbutyl)-4-(4-(pyridin-4-ylmethoxy)phenyl)-1H-imidazole-1-carboxamide (AC2020601-0323)

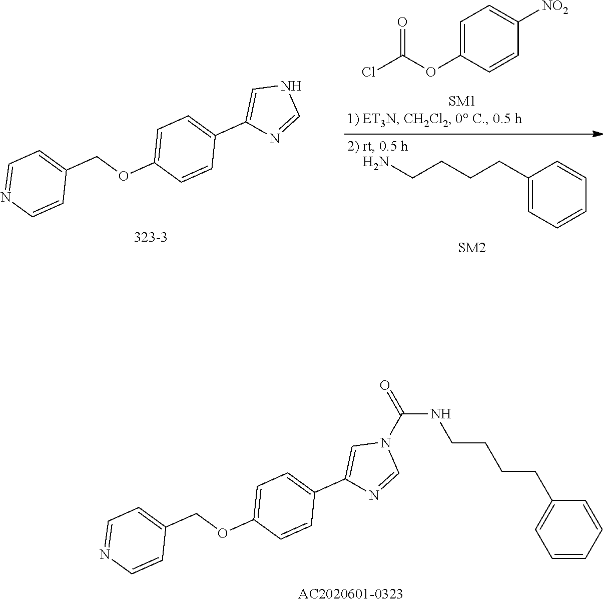 [SMILES: O=C(Cl)Oc1ccc([N+](=O)[O-])cc1 ; c1cc(COc2ccc(-c3c[nH]cn3)cc2)ccn1 ; NCCCCc1ccccc1 ; O=C(NCCCCc1ccccc1)n1cnc(-c2ccc(OCc3ccncc3)cc2)c1]

To a solution of 323-3 (380 mg, 1.5 mmol) in DCM (5 mL) was added SM1 (302.0 mg, 1.5 mmol) and Et₃N (5 mL) at 0° C. and stirred for 0.5 hour. Then the reaction mixture was allowed to warm to rt and SM2 (225.0 mg, 1.5 mmol) was added. After 0.5 hour stirring, the reaction mixture was quenched with water, extracted with DCM (20 mL), the combined organic layer was washed with brine, dried over anhydrous Na₂SO₄ and evaporated under reduced pressure. The crude was purified by silica gel chromatography (10% MeOH/DCM) to afford AC2020601-0323 (21.0 mg, yield: 3.3%) as a white solid. ¹H NMR (400 MHz, CDCl₃) δ 8.61 (dd, J=4.4 Hz, J=1.6 Hz, 2H), 8.20 (d, J=1.2 Hz, 1H), 7.69 (dd, J=6.8 Hz, J=2.0 Hz, 2H), 7.51 (d, J=1.2 Hz, 1H), 7.37 (d, J=6.0 Hz, 2H), 7.29-7.30 (m, 2H), 7.17-7.21 (m, 3H), 6.97 (dd, J=6.8 Hz, J=2.0 Hz, 2H), 6.16 (t, J=5.2 Hz, 1H), 5.11 (s, 2H), 3.43-3.48 (m, 2H), 2.67 (t, J=7.2 Hz, 2H), 1.64-1.75 (m, 4H). LC-MS m/z: 427.1 [M+H]⁺. HPLC Purity (254 nm): 95.65%; t_{R}=9.325 min.

AC2020601-0324

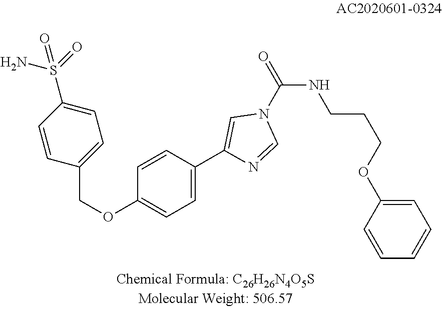 [SMILES: NS(=O)(=O)c1ccc(COc2ccc(-c3cn(C(=O)NCCCOc4ccccc4)cn3)cc2)cc1]

### The Synthesis of N-(4-phenylbutyl)-4-(4-((4-sulfamoylbenzyl)oxy)phenyl)-1H-imidazole-1-carboxamide (AC2020601-0324)

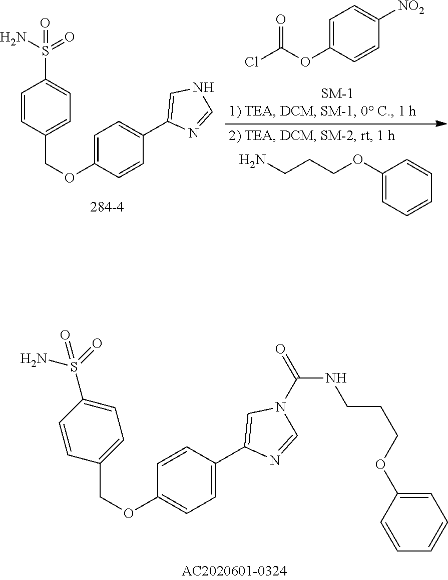 [SMILES: NS(=O)(=O)c1ccc(COc2ccc(-c3c[nH]cn3)cc2)cc1 ; O=C(Cl)Oc1ccc([N+](=O)[O-])cc1 ; NCCCOc1ccccc1 ; NS(=O)(=O)c1ccc(COc2ccc(-c3cn(C(=O)NCCCOc4ccccc4)cn3)cc2)cc1]

To a solution of 284-4 (200 mg, 0.61 mmol) in DCM (5 mL) was added 4-nitrophenyl carbonochloridate (147 mg, 0.73 mmol) and Et₃N (5 mL) at 0° C. and stirred for 1 hour. Then the reaction mixture was allowed to warm to room temperature and 3-phenoxypropan-1-amine (110 mg, 0.73 mmol) was added. After 1 hour stirring, the reaction mixture was quenched with water, extracted with DCM (20 mL), the combined organic layer was washed with brine, dried over anhydrous Na₂SO₄ and evaporated under reduced pressure. The crude was purified by silica gel chromatography (5% MeOH/DCM) to afford AC2020601-0324 (14 mg, yield: 5%) as a white solid. ¹H NMR (400 MHz, DMSO-d₆) δ 8.96 (br, 1H), 8.36 (d, J=4.0 Hz, 1H), 8.20 (d, J=10.4 Hz, 1H), 7.85 (d, J=8.4 Hz, 2H), 7.72 (d, J=8.4 Hz, 2H), 7.64 (d, J=8.0 Hz, 2H), 7.39 (s, 2H), 7.28 (t, J=8.0 Hz, 2H), 7.05 (d, J=8.4 Hz, 2H), 6.90-6.95 (m, 3H), 5.23 (s, 2H), 4.06 (t, J=6.0 Hz, 2H), 3.41-3.46 (m, 2H), 2.00-2.08 (m, 2H). LC-MS m/z: 506.9 [M+H]⁺. HPLC Purity (254 nm): 97.54%; t_{R}=8.676 min.

AC2020601-0327

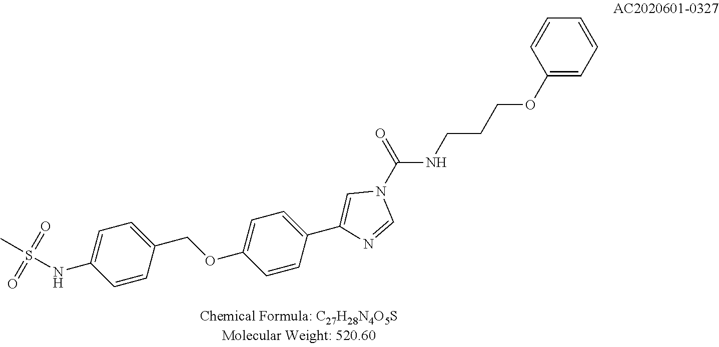 [SMILES: CS(=O)(=O)Nc1ccc(COc2ccc(-c3cn(C(=O)NCCCOc4ccccc4)cn3)cc2)cc1]

### The Synthesis of 4-(4-((4-(methylsulfonamido)benzyl)oxy)phenyl)-N-(3-phenoxypropyl)-1H-imidazole-1-carboxamide (AC2020601-0327)

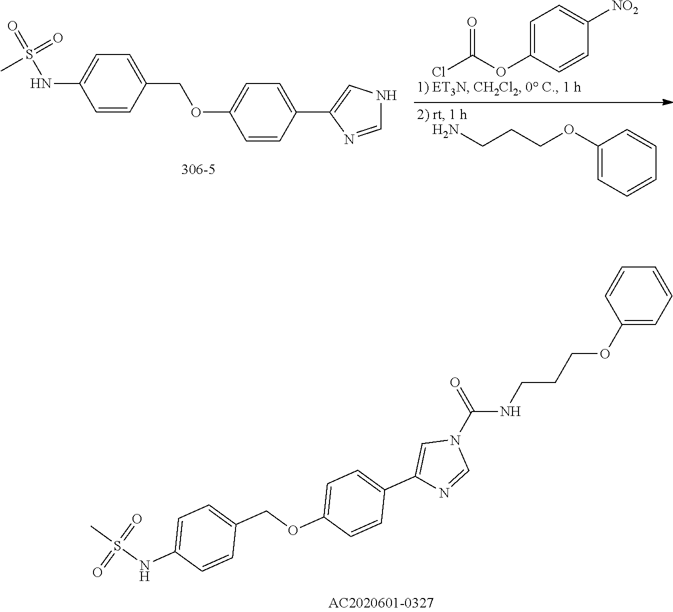 [SMILES: CS(=O)(=O)Nc1ccc(COc2ccc(-c3c[nH]cn3)cc2)cc1 ; O=C(Cl)Oc1ccc([N+](=O)[O-])cc1 ; NCCCOc1ccccc1 ; CS(=O)(=O)Nc1ccc(COc2ccc(-c3cn(C(=O)NCCCOc4ccccc4)cn3)cc2)cc1]

To a solution of 306-5 (100 mg, 0.29 mmol) in DCM (5 mL) was added 4-nitrophenyl carbonochloridate (70 mg, 0.35 mmol) and Et₃N (5 mL) at 0° C. and stirred for 1 hour. Then the reaction mixture was allowed to warm to room temperature and 3-phenoxypropan-1-amine (52 mg, 0.35 mmol) was added. After 1 hour stirring, the reaction mixture was quenched with water, extracted with DCM (20 mL), the combined organic layer was washed with brine, dried over anhydrous Na₂SO₄ and evaporated under reduced pressure. The crude was purified by silica gel chromatography (5% MeOH/DCM) to afford AC2020601-0327 (7 mg, yield: 5%) as a white solid. ¹H NMR (400 MHz, DMSO-d₆) δ 9.79 (s, 1H), 8.60 (d, J=4.4 Hz, 1H), 8.27 (s, 1H), 8.00 (s, 1H), 7.72 (d, J=8.4 Hz, 2H), 7.43 (d, J=8.4 Hz, 2H), 7.22-7.31 (m, 4H), 7.03 (d, J=8.8 Hz, 2H), 6.91-6.95 (m, 3H), 5.06 (s, 2H), 4.06 (t, J=6.0 Hz, 2H), 3.42-3.47 (m, 2H), 2.99 (s, 3H), 2.02 (t, J=6.0 Hz, 2H). LC-MS m/z: 520.9 [M+H]⁺. HPLC Purity (254 nm): 94.99%; t_{R}=8.979 min.

AC2020601-0328

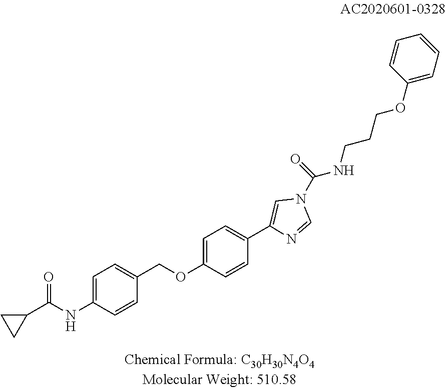 [SMILES: O=C(Nc1ccc(COc2ccc(-c3cn(C(=O)NCCCOc4ccccc4)cn3)cc2)cc1)C1CC1]

### The Synthesis of tert-butyl 4-(4-((4-(cyclopropanecarboxamido)benzyl)oxy)phenyl)-1H-imidazole-1-carboxylate (328-4)

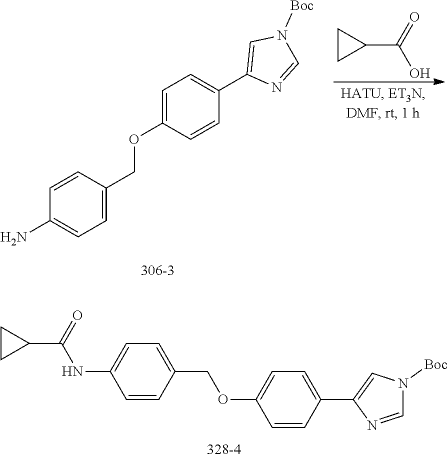

To a suspension of cyclopropanecarboxylic acid (71 mg, 0.82 mmol) in DMF (10 mL) was added Et₃N (111 mg, 1.09 mmol) and HATU (312 mg, 0.82 mmol). The reaction mixture was stirred at room temperature for 0.5 hour, then 306-3 (200 mg, 0.55 mmol) was added. The reaction mixture was stirred at room temperature for 1 hour until the reaction was complete (by LCMS). Water was added and the reaction mixture was extracted with EtOAc (50 mL), the combined organic layer was washed with brine, dried over anhydrous Na₂SO₄ and evaporated under reduced pressure. The crude was purified by silica gel chromatography (5% MeOH/DCM) to afford 328-4 (150 mg, yield: 6₃%) as a white solid.

### The Synthesis of N-(4-((4-(1H-imidazol-4-yl)phenoxy)methyl)phenyl)cyclopropanecarboxamide (328-5)

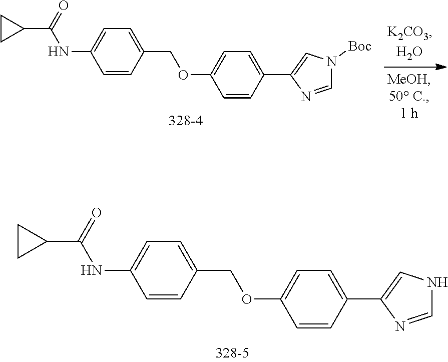 [SMILES: CC(C)(C)OC(=O)n1cnc(-c2ccc(OCc3ccc(NC(=O)C4CC4)cc3)cc2)c1 ; O=C(Nc1ccc(COc2ccc(-c3c[nH]cn3)cc2)cc1)C1CC1]

To a stirred solution of 328-4 (200 mg, 0.46 mmol) in MeOH (10 mL) and H₂O (1 mL) was added K₂CO₃ (128 mg, 0.92 mmol). The reaction mixture was stirred at 50° C. for 1 hour until the reaction was complete (by LCMS). The reaction mixture was quenched with water, extracted with EA (20 mL), the combined organic layer was washed with brine, dried over anhydrous Na₂SO₄. The mixture was concentrated under reduced pressure to afford 328-5 (110 mg, yield: 72%) as a white solid, which was used to the next step without purification.

### The Synthesis of 4-(4-((4-(cyclopropanecarboxamido)benzyl)oxy)phenyl)-N-(3-phenoxypropyl)-1H-imidazole-1-carboxamide (AC2020601-0328)

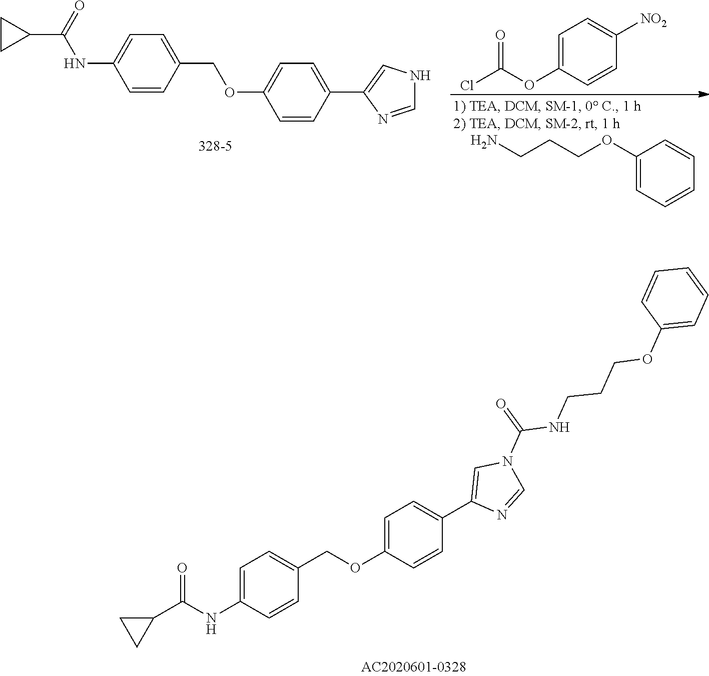 [SMILES: O=C(Nc1ccc(COc2ccc(-c3c[nH]cn3)cc2)cc1)C1CC1 ; O=C(Cl)Oc1ccc([N+](=O)[O-])cc1 ; NCCCOc1ccccc1 ; O=C(Nc1ccc(COc2ccc(-c3cn(C(=O)NCCCOc4ccccc4)cn3)cc2)cc1)C1CC1]

To a solution of 328-5 (110 mg, 0.33 mmol) in DCM (5 mL) was added 4-nitrophenyl carbonochloridate (80 mg, 0.40 mmol) and Et₃N (5 mL) at 0° C. and stirred for 1 hour. Then the reaction mixture was allowed to warm to room temperature and 3-phenoxypropan-1-amine (60 mg, 0.40 mmol) was added. After 1 hour stirring, the reaction mixture was quenched with water, extracted with DCM (20 mL), the combined organic layer was washed with brine, dried over anhydrous Na₂SO₄ and evaporated under reduced pressure. The crude was purified by silica gel chromatography (5% MeOH/DCM) to afford AC2020601-0328 (27 mg, yield: 16%) as a white solid. ¹H NMR (400 MHz, DMSO-d₆) δ 10.24 (s, 1H), 8.58 (t, J=4.8 Hz, 1H), 8.27 (s, 1H), 8.00 (s, 1H), 7.71 (d, J=8.4 Hz, 2H), 7.60 (d, J=8.0 Hz, 2H), 7.37 (d, J=8.0 Hz, 2H), 7.27-7.30 (m, 2H), 7.03 (d, J=8.4 Hz, 2H), 6.93-6.95 (m, 3H), 5.04 (s, 2H), 4.06 (t, J=5.6 Hz, 2H), 3.42-3.45 (m, 2H), 2.00-2.03 (m, 2H), 1.76-1.79 (m, 1H), 0.78-0.80 (m, 4H). LC-MS m/z: 511.0 [M+H]⁺. HPLC Purity (254 nm): 96.95%; t_{R}=9.353 min.

AC2020601-0339

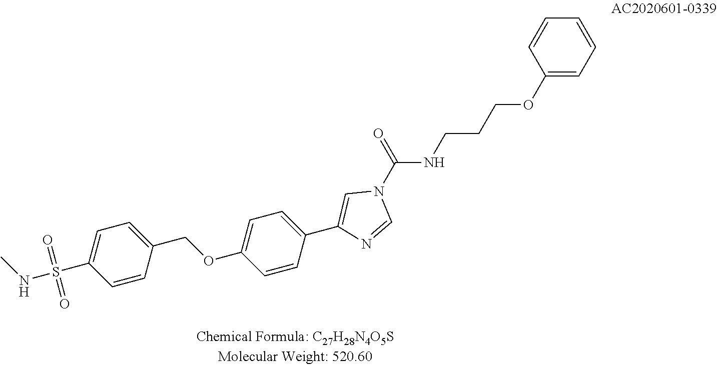 [SMILES: CNS(=O)(=O)c1ccc(COc2ccc(-c3cn(C(=O)NCCCOc4ccccc4)cn3)cc2)cc1]

### The Synthesis of 4-(bromomethyl)-N-methylbenzenesulfonamide (339-2)

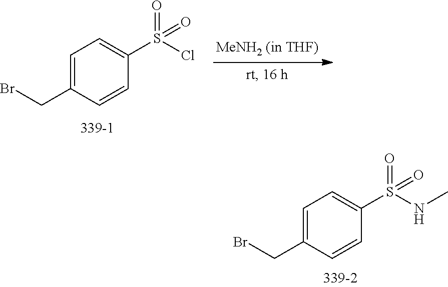

A solution of 339-1 (500 mg, 1.85 mmol) in MeNH₂ (10 mL, 1 N in THF) was stirred at room temperature for 16 hours until the reaction was complete (by LCMS). The mixture was concentrated under reduced pressure to afford 339-2 (180 mg, yield: 37%) as colorless oil, which was used to the next step without purification.

### The Synthesis of tert-butyl 4-(4-((4-(N-methylsulfamoyl)benzyl)oxy)phenyl)-1H-imidazole-1-carboxylate (339-3)

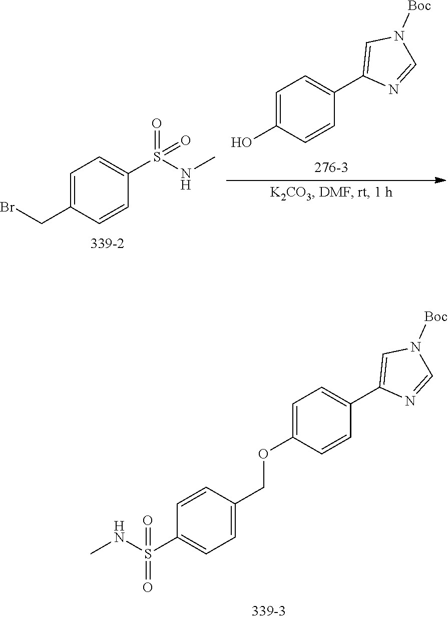

To a suspension of 276-3 (260 mg, 1.00 mmol) in DMF (10 mL) was added K₂CO₃ (276 mg, 2.00 mmol) and 339-2 (264 mg, 1.00 mmol). The reaction mixture was stirred at room temperature for 3 hours until the reaction was complete (by LCMS). Saturated NaCl solution was added and the reaction mixture was extracted with EtOAc (50 mL), the combined organic layer was washed with brine, dried over anhydrous Na₂SO₄ and evaporated under reduced pressure. The crude was purified by silica gel chromatography (5% MeOH/DCM) to afford 339-3 (200 mg, yield: 45%) as a yellow solid.

### The Synthesis of 4-((4-(1H-imidazol-4-yl)phenoxy)methyl)-N-methylbenzenesulfonamide (339-4)

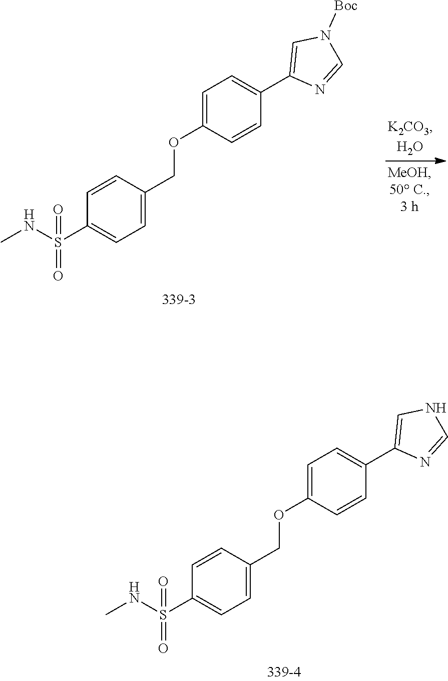 [SMILES: CNS(=O)(=O)c1ccc(COc2ccc(-c3cn(C(=O)OC(C)(C)C)cn3)cc2)cc1 ; CNS(=O)(=O)c1ccc(COc2ccc(-c3c[nH]cn3)cc2)cc1]

To a stirred solution of 339-3 (200 mg, 0.45 mmol) in MeOH (10 mL) and H₂O (1 mL) was added K₂CO₃ (125 mg, 0.90 mmol). The reaction mixture was stirred at 50° C. for 3 hours until the reaction was complete (by LCMS). The reaction mixture was quenched with water, extracted with EA (20 mL), the combined organic layer was washed with brine, dried over anhydrous Na₂SO₄. The mixture was concentrated under reduced pressure to afford 339-4 (100 mg, yield: 65%) as a white solid, which was used to the next step without purification.

### The Synthesis of 4-(4-((4-(N-methylsulfamoyl)benzyl)oxy)phenyl)-N-(3-phenoxypropyl)-1H-imidazole-1-carboxamide (AC2020601-0339)

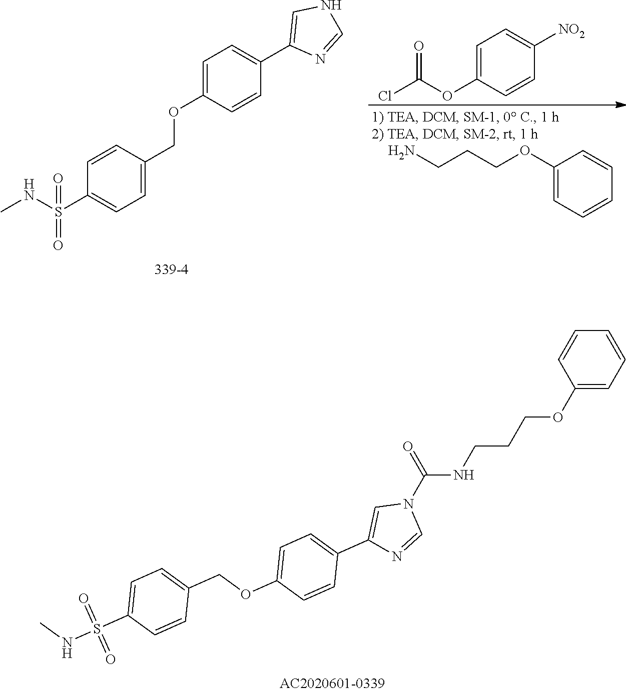 [SMILES: CNS(=O)(=O)c1ccc(COc2ccc(-c3c[nH]cn3)cc2)cc1 ; O=C(Cl)Oc1ccc([N+](=O)[O-])cc1 ; NCCCOc1ccccc1 ; CNS(=O)(=O)c1ccc(COc2ccc(-c3cn(C(=O)NCCCOc4ccccc4)cn3)cc2)cc1]

To a solution of 339-4 (100 mg, 0.29 mmol) in DCM (5 mL) was added 4-nitrophenyl carbonochloridate (70 mg, 0.35 mmol) and Et₃N (5 mL) at 0° C. and stirred for 1 hour. Then the reaction mixture was allowed to warm to room temperature and 3-phenoxypropan-1-amine (52 mg, 0.35 mmol) was added. After 1 hour stirring, the reaction mixture was quenched with water, extracted with DCM (20 mL), the combined organic layer was washed with brine, dried over anhydrous Na₂SO₄ and evaporated under reduced pressure. The crude was purified by silica gel chromatography (5% MeOH/DCM) to afford AC2020601-0339 (10 mg, yield: 7%) as a white solid. ¹H NMR (400 MHz, DMSO-d₆) δ 8.59 (t, J=5.6 Hz, 1H), 8.27 (s, 1H), 8.01 (s, 1H), 7.80 (d, J=8.0 Hz, 2H), 7.74 (d, J=8.4 Hz, 2H), 7.67 (d, J=8.4 Hz, 2H), 7.44-7.48 (m, 1H), 7.27-7.31 (m, 2H), 7.06 (d, J=8.8 Hz, 2H), 6.91-6.95 (m, 3H), 5.24 (s, 2H), 4.06 (t, J=6.0 Hz, 2H), 3.42-3.47 (m, 2H), 2.41 (d, J=4.8 Hz, 3H), 1.98-2.05 (m, 2H). LC-MS m/z: 521.2 [M+H]⁺. HPLC Purity (254 nm): 96.33%; t_{R}=9.036 min.

AC2020601-0342

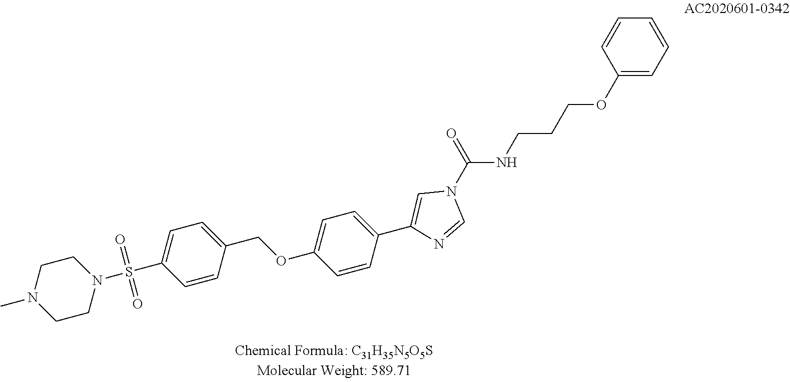 [SMILES: CN1CCN(S(=O)(=O)c2ccc(COc3ccc(-c4cn(C(=O)NCCCOc5ccccc5)cn4)cc3)cc2)CC1]

### The Synthesis of 4-(bromomethyl)-N-methylbenzenesulfonamide (342-2)

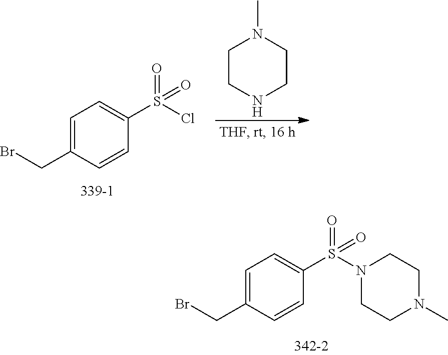

A solution of 339-1 (500 mg, 1.85 mmol) in 1-methylpiperazine (5 mL, 0.37 N in THF) was stirred at room temperature for 16 hours until the reaction was complete (by LCMS). The mixture was concentrated under reduced pressure to afford 342-2 (150 mg, yield: 24%) as colorless oil, which was used to the next step without purification.

### The Synthesis of tert-butyl 4-(4-((4-((4-methylpiperazin-1-yl)sulfonyl)benzyl)oxy)phenyl)-1H-imidazole-1-carboxylate (342-3)

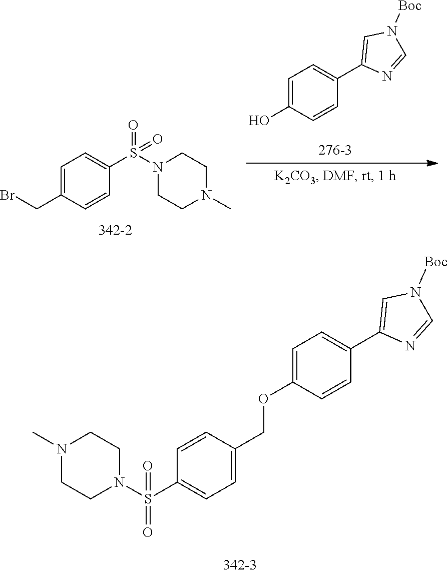 [SMILES: CC(C)(C)OC(=O)n1cnc(-c2ccc(O)cc2)c1 ; CN1CCN(S(=O)(=O)c2ccc(CBr)cc2)CC1 ; CN1CCN(S(=O)(=O)c2ccc(COc3ccc(-c4cn(C(=O)OC(C)(C)C)cn4)cc3)cc2)CC1]

To a suspension of 276-3 (260 mg, 1.00 mmol) in DMF (10 mL) was added K₂CO₃ (276 mg, 2.00 mmol) and 342-2 (333 mg, 1.00 mmol). The reaction mixture was stirred at room temperature for 1 hour until the reaction was complete (by LCMS). Water was added and the reaction mixture was extracted with EtOAc (50 mL), the combined organic layer was washed with brine, dried over anhydrous Na₂SO₄ and evaporated under reduced pressure. The crude was purified by silica gel chromatography (5% MeOH/DCM) to afford 342-3 (100 mg, yield: 20%) as a yellow solid.

### The Synthesis of 4-((4-((4-(1H-imidazol-4-yl)phenoxy)methyl)phenyl)sulfonyl)morpholine (342-4)

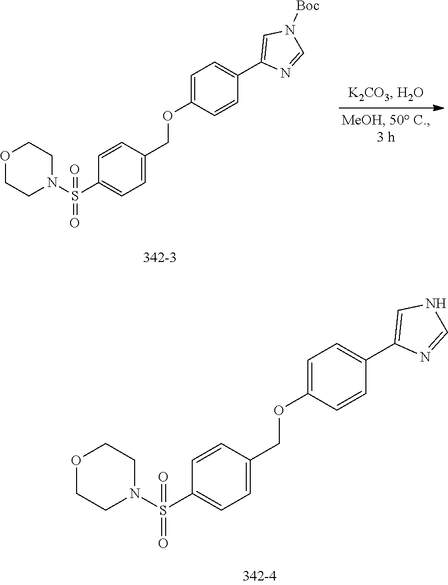 [SMILES: CC(C)(C)OC(=O)n1cnc(-c2ccc(OCc3ccc(S(=O)(=O)N4CCOCC4)cc3)cc2)c1 ; O=S(=O)(c1ccc(COc2ccc(-c3c[nH]cn3)cc2)cc1)N1CCOCC1]

To a stirred solution of 342-3 (103 mg, 0.20 mmol) in MeOH (10 mL) and H₂O (1 mL) was added K₂CO₃ (55 mg, 0.40 mmol). The reaction mixture was stirred at 50° C. for 3 hours until the reaction was complete (by LCMS). The reaction mixture was quenched with water, extracted with EA (20 mL), the combined organic layer was washed with brine, dried over anhydrous Na₂SO₄. The mixture was concentrated under reduced pressure to afford 342-4 (60 mg, yield: 71%) as a white solid, which was used to the next step without purification.

### The Synthesis of 4-(4-((4-((4-methylpiperazin-1-yl)sulfonyl)benzyl)oxy)phenyl)-N-(3-phenoxypropyl)-1H-imidazole-1-carboxamide (AC2020601-0342)

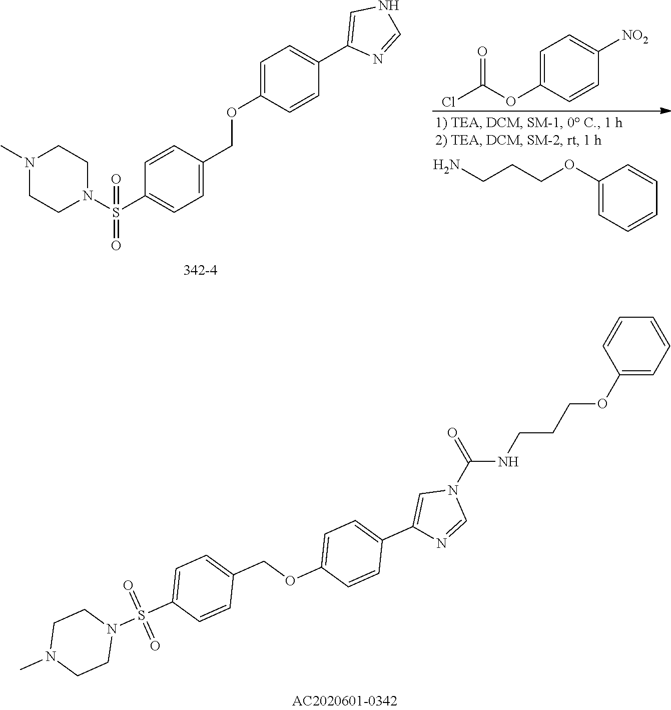 [SMILES: CN1CCN(S(=O)(=O)c2ccc(COc3ccc(-c4c[nH]cn4)cc3)cc2)CC1 ; O=C(Cl)Oc1ccc([N+](=O)[O-])cc1 ; NCCCOc1ccccc1 ; CN1CCN(S(=O)(=O)c2ccc(COc3ccc(-c4cn(C(=O)NCCCOc5ccccc5)cn4)cc3)cc2)CC1]

To a solution of 342-4 (62 mg, 0.15 mmol) in DCM (5 mL) was added 4-nitrophenyl carbonochloridate (36 mg, 0.18 mmol) and Et₃N (5 mL) at 0° C. and stirred for 1 hour. Then the reaction mixture was allowed to warm to room temperature and 3-phenoxypropan-1-amine (27 mg, 0.18 mmol) was added. After 1 hour stirring, the reaction mixture was quenched with water, extracted with DCM (20 mL), the combined organic layer was washed with brine, dried over anhydrous Na₂SO₄ and evaporated under reduced pressure. The crude was purified by silica gel chromatography (5% MeOH/DCM) to afford AC2020601-0342 (5 mg, yield: 6%) as a white solid. ¹H NMR (400 MHz, DMSO-d₆) δ 8.60 (t, J=5.2 Hz, 1H), 8.28 (s, 1H), 8.02 (s, 1H), 7.72-7.78 (m, 6H), 7.28 (t, J=7.6 Hz, 2H), 7.06 (d, J=8.4 Hz, 2H), 6.91-6.95 (m, 3H), 5.27 (s, 2H), 4.06 (t, J=5.6 Hz, 2H), 3.42-3.46 (m, 2H), 2.89 (s, 4H), 2.36 (s, 4H), 2.14 (s, 3H), 2.02 (t, J=6.4 Hz, 2H). LC-MS m/z: 590.3 [M+H]⁺. HPLC Purity (254 nm): 94.85%; t_{R}=9.339 min.

AC2020601-0347

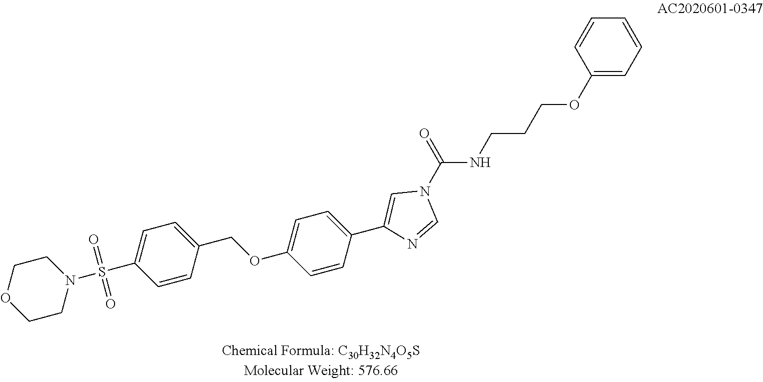 [SMILES: O=C(NCCCOc1ccccc1)n1cnc(-c2ccc(OCc3ccc(S(=O)(=O)N4CCOCC4)cc3)cc2)c1]

### The Synthesis of 4-(bromomethyl)-N-methylbenzenesulfonamide (347-2)

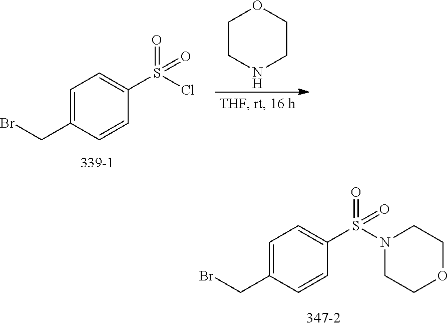

A solution of 339-1 (500 mg, 1.85 mmol) in morpholine (5 mL, 0.37 N in THF) was stirred at room temperature for 16 hours until the reaction was complete (by LCMS). The mixture was concentrated under reduced pressure to afford 347-2 (180 mg, yield: 30%) as colorless oil, which was used to the next step without purification.

### The Synthesis of tert-butyl 4-(4-((4-(morpholinosulfonyl)benzyl)oxy)phenyl)-1H-imidazole-1-carboxylate (347-3)

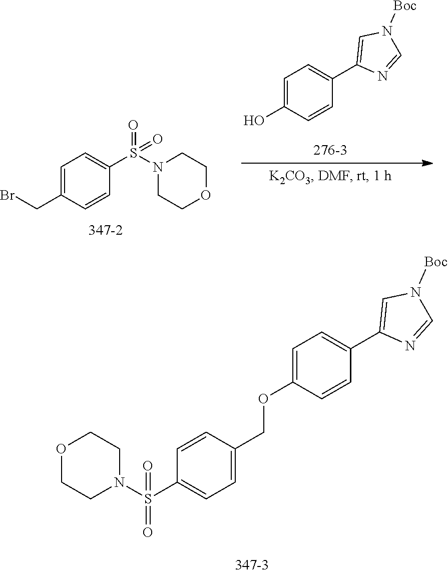 [SMILES: CC(C)(C)OC(=O)n1cnc(-c2ccc(O)cc2)c1 ; O=S(=O)(c1ccc(CBr)cc1)N1CCOCC1 ; CC(C)(C)OC(=O)n1cnc(-c2ccc(OCc3ccc(S(=O)(=O)N4CCOCC4)cc3)cc2)c1]

To a suspension of 276-3 (260 mg, 1.00 mmol) in DMF (10 mL) was added K₂CO₃ (276 mg, 2.00 mmol) and 347-2 (320 mg, 1.00 mmol). The reaction mixture was stirred at room temperature for 1 hour until the reaction was complete (by LCMS). Saturated NaCl solution was added and the reaction mixture was extracted with EtOAc (50 mL), the combined organic layer was washed with brine, dried over anhydrous Na₂SO₄ and evaporated under reduced pressure. The crude was purified by silica gel chromatography (5% MeOH/DCM) to afford 347-3 (100 mg, yield: 20%) as a yellow solid.

### The Synthesis of 4-((4-((4-(1H-imidazol-4-yl)phenoxy)methyl)phenyl)sulfonyl)morpholine (347-4)

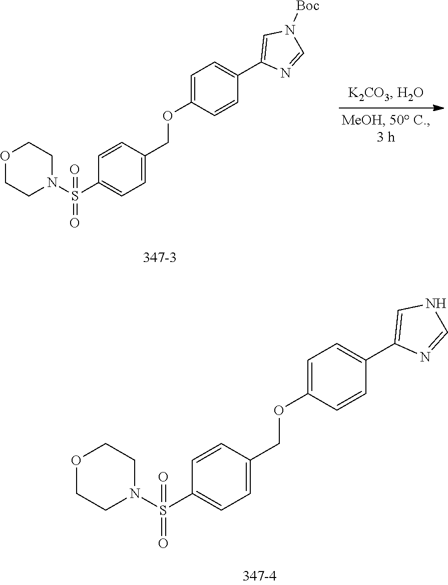 [SMILES: CC(C)(C)OC(=O)n1cnc(-c2ccc(OCc3ccc(S(=O)(=O)N4CCOCC4)cc3)cc2)c1 ; O=S(=O)(c1ccc(COc2ccc(-c3c[nH]cn3)cc2)cc1)N1CCOCC1]

To a stirred solution of 347-3 (100 mg, 0.20 mmol) in MeOH (10 mL) and H₂O (1 mL) was added K₂CO₃ (55 mg, 0.40 mmol). The reaction mixture was stirred at 50° C. for 3 hours until the reaction was complete (by LCMS). The reaction mixture was quenched with water, extracted with EA (20 mL), the combined organic layer was washed with brine, dried over anhydrous Na₂SO₄. The mixture was concentrated under reduced pressure to afford 347-4 (60 mg, yield: 75%) as a white solid, which was used to the next step without purification.

### The Synthesis of 4-(4-((4-(morpholinosulfonyl)benzyl)oxy)phenyl)-N-(3-phenoxypropyl)-1H-imidazole-1-carboxamide (AC2020601-0347)

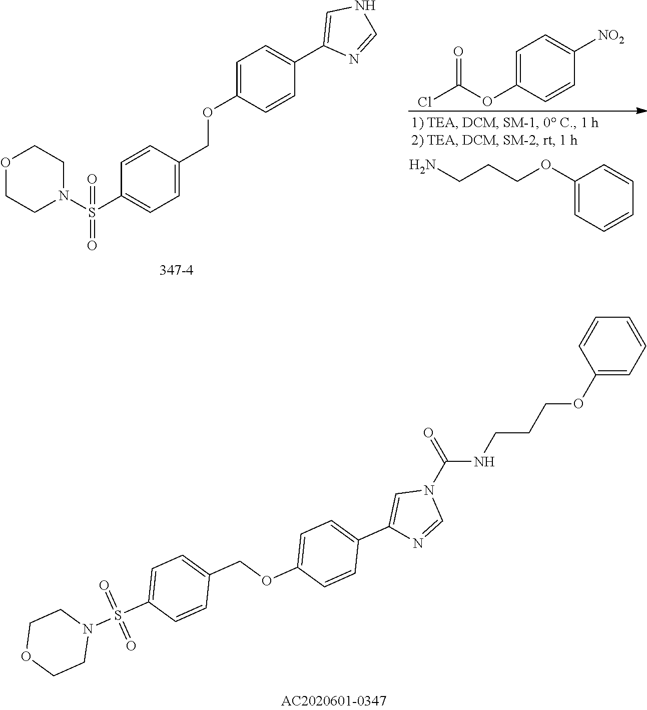 [SMILES: O=S(=O)(c1ccc(COc2ccc(-c3c[nH]cn3)cc2)cc1)N1CCOCC1 ; O=C(Cl)Oc1ccc([N+](=O)[O-])cc1 ; NCCCOc1ccccc1 ; O=C(NCCCOc1ccccc1)n1cnc(-c2ccc(OCc3ccc(S(=O)(=O)N4CCOCC4)cc3)cc2)c1]

To a solution of 347-4 (60 mg, 0.15 mmol) in DCM (5 mL) was added 4-nitrophenyl carbonochloridate (36 mg, 0.18 mmol) and Et₃N (5 mL) at 0° C. and stirred for 1 hour. Then the reaction mixture was allowed to warm to room temperature and 3-phenoxypropan-1-amine (27 mg, 0.18 mmol) was added. After 1 hour stirring, the reaction mixture was quenched with water, extracted with DCM (20 mL), the combined organic layer was washed with brine, dried over anhydrous Na₂SO₄ and evaporated under reduced pressure. The crude was purified by silica gel chromatography (5% MeOH/DCM) to afford AC2020601-0347 (5 mg, yield: 6%) as a white solid. ¹H NMR (400 MHz, DMSO-d₆) δ 8.59 (t, J=5.2 Hz, 1H), 8.28 (s, 1H), 8.02 (s, 1H), 7.73-7.91 (m, 6H), 7.29 (t, J=7.6 Hz, 2H), 7.07 (d, J=8.8 Hz, 2H), 6.91-6.95 (m, 3H), 5.28 (s, 2H), 4.06 (t, J=5.6 Hz, 2H), 3.63 (s, 4H), 3.42-3.45 (m, 2H), 2.87 (s, 4H), 2.02 (t, J=6.0 Hz, 2H). LC-MS m/z: 577.3 [M+H]⁺. HPLC Purity (254 nm): 97.14%; t_{R}=9.466 min.

AC2020601-0348

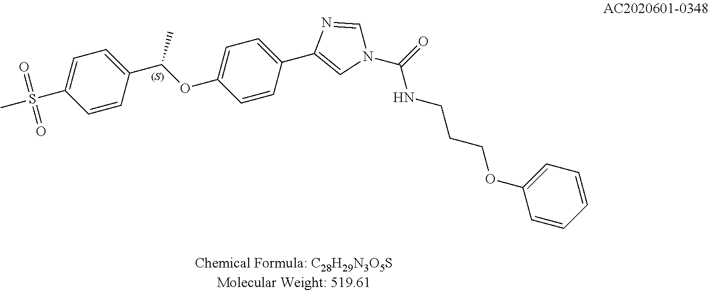

### The Synthesis of 1-(4-(methylsulfonyl)phenyl)ethanol (348-1-2)

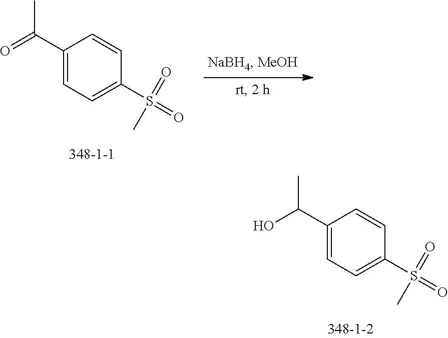

To a suspension of 348-1-1 (3 g, 15.1 mmol) in MeOH (30 mL) was added NaBH₄ (5.7 g, 151 mmol). The reaction mixture was stirred at room temperature for 2 hours until the reaction was complete (by LCMS). Then the reaction mixture was poured into water (20 mL), extracted with EtOAc (20 mL), the combined organic layer was washed with water and brine, dried over anhydrous Na₂SO₄, filtered and evaporated under reduced pressure. The crude was purified by silica gel chromatography (5-20% MeOH in CH₂Cl₂) to get 348-1-2 (2.5 g, yield: 82%) as a white solid.

### The Synthesis of tert-butyl 4-(4-(1-(4-(methylsulfonyl)phenyl)ethoxy)phenyl)-1H-imidazole-1-carboxylate (348-2)

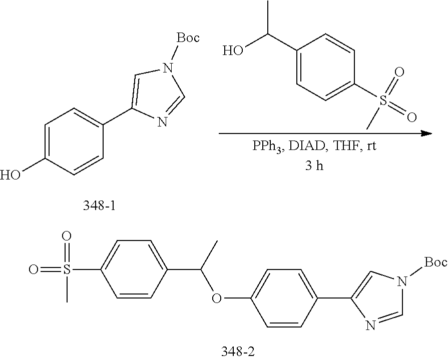 [SMILES: CC(C)(C)OC(=O)n1cnc(-c2ccc(O)cc2)c1 ; CC(O)c1ccc(S(C)(=O)=O)cc1 ; CC(Oc1ccc(-c2cn(C(=O)OC(C)(C)C)cn2)cc1)c1ccc(S(C)(=O)=O)cc1]

To a suspension of 348-1 (1.0 g, 3.8 mmol) in THF (20 mL) was added 1-(4-(methylsulfonyl)phenyl)ethanol (760.0 mg, 3.8 mmol), PPh₃ (1.2 g, 4.56 mmol) and DIAD (998.0 mg, 4.94 mmol). The reaction mixture was stirred at room temperature for 3 hours until the reaction was complete (by LCMS). Saturated NaCl solution was added and the reaction mixture was extracted with EtOAc (20 mL), the combined organic layer was washed with brine, dried over anhydrous Na₂SO₄ and evaporated under reduced pressure. The crude was purified by silica gel chromatography (10% MeOH/DCM) to afford 348-2 (1.2 g, yield: 71%) as yellow oil.

### The Synthesis of (S)-4-(4-(1-(4-(methylsulfonyl)phenyl)ethoxy)phenyl)-1H-imidazole (348-3) and (R)-4-(4-(1-(4-(methylsulfonyl)phenyl)ethoxy)phenyl)-1H-imidazole (349-3)

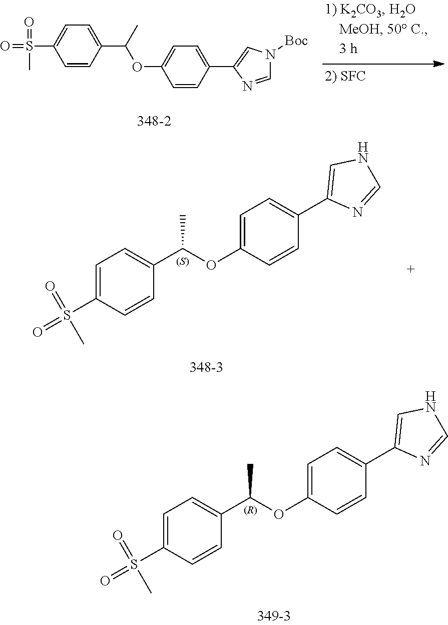

To a stirred solution of 348-2 (1.2 g, 2.7 mmol) in MeOH (20 mL) and H₂O (4 mL) was added K₂CO₃ (745.0 mg, 5.4 mmol). The reaction mixture was allowed to warm to 50° C. for 3 hours until the reaction was complete (by LCMS). The mixture was concentrated under reduced pressure, then further purification by SFC to afford 348-3 (342 mg, yield: 37%) as yellow oil and 349-3 (342 mg, yield: 37%) as yellow oil. Note: The absolute configurations are tentatively assigned.

### The Synthesis of (S)-4-(4-(1-(4-(methylsulfonyl)phenyl)ethoxy)phenyl)-N-(3-phenoxypropyl)-1H-imidazole-1-carboxamide (AC2020601-0348)

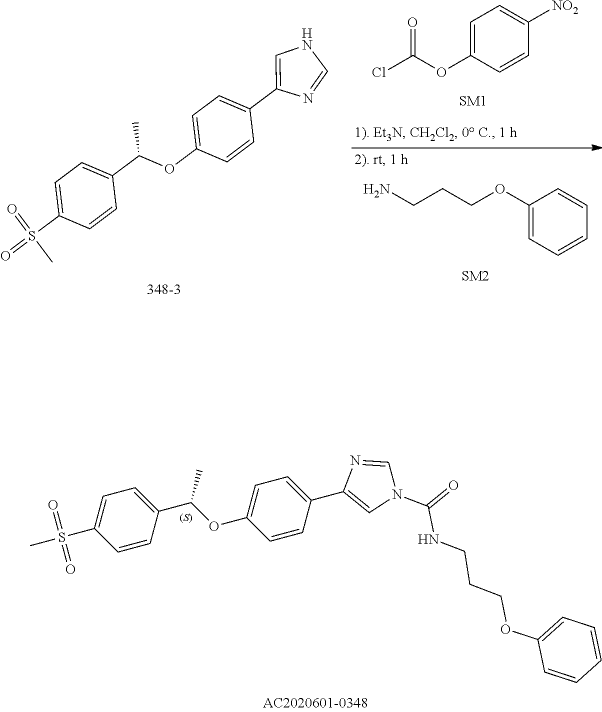

To a solution of 348-3 (342 mg, 1.0 mmol) in DCM (10 mL) was added SM1 (302.0 mg, 1.5 mmol) and Et₃N (10 mL) at 0° C. and stirred for 1 hour. Then the reaction mixture was allowed to warm to rt and SM2 (301.0 mg, 2.0 mmol) was added. After 1 hour stirring, the reaction mixture was quenched with water, extracted with DCM (20 mL), the combined organic layer was washed with brine, dried over anhydrous Na₂SO₄ and evaporated under reduced pressure. The crude was purified by silica gel chromatography (10% MeOH/DCM) to afford AC2020601-0348 (8.48 mg, yield: 4.8%) as a white solid. ¹H NMR (400 MHz, DMSO-d₆) δ 8.57 (t, J=5.2 Hz, 1H), 8.24 (s, 1H), 7.90-7.95 (m, 3H), 7.69 (d, J=8.4 Hz, 2H), 7.64 (d, J=8.4 Hz, 2H), 7.28 (t, J=8.0 Hz, 2H), 6.90-6.99 (m, 5H), 5.66-5.71 (m, 1H), 4.05 (t, J=6.0 Hz, 2H), 3.40-3.48 (m, 2H), 3.20 (s, 3H), 2.00 (t, J=6.4 Hz, 2H), 1.57 (d, J=6.4 Hz, 3H). LC-MS m/z: 519.9 [M+H]⁺. HPLC Purity (254 nm): 97.86%; t_{R}=8.422 min.

AC2020601-0349

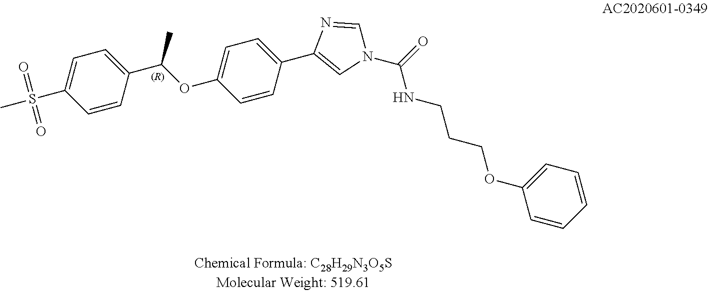

### The Synthesis of (R)-4-(4-(1-(4-(methylsulfonyl)phenyl)ethoxy)phenyl)-N-(3-phenoxypropyl)-1H-imidazole-1-carboxamide (AC2020601-0349)

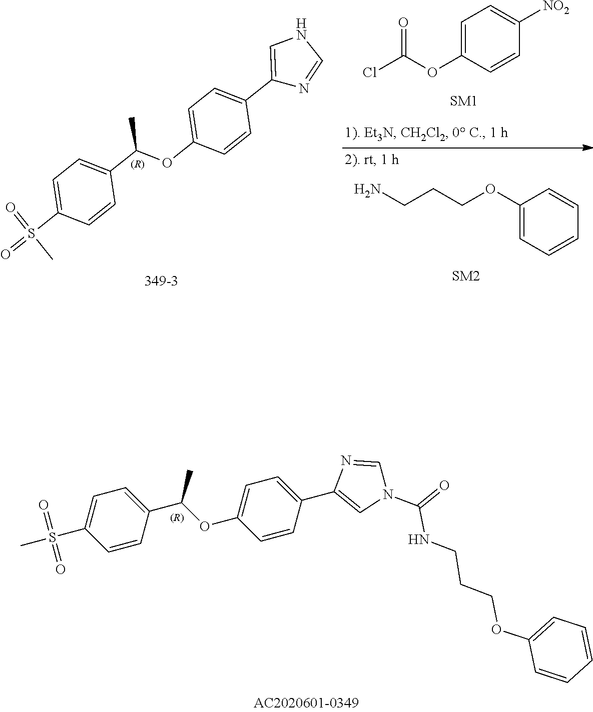

To a solution of 349-3 (342.0 mg, 1.0 mmol) in DCM (10 mL) was added SM1 (302.0 mg, 1.5 mmol) and Et₃N (10 mL) at 0° C. and stirred for 1 hour. Then the reaction mixture was allowed to warm to rt and SM2 (301.0 mg, 2.0 mmol) was added. After 1 hour stirring, the reaction mixture was quenched with water, extracted with DCM (20 mL), the combined organic layer was washed with brine, dried over anhydrous Na₂SO₄ and evaporated under reduced pressure. The crude was purified by silica gel chromatography (10% MeOH/DCM) to afford AC2020601-0349 (5.04 mg, yield: 3.7%) as a white solid. ¹H NMR (400 MHz, DMSO-d₆) δ 8.57 (t, J=5.2 Hz, 1H), 8.24 (s, 1H), 7.90-7.95 (m, 3H), 7.69 (d, J=8.4 Hz, 2H), 7.64 (d, J=8.4 Hz, 2H), 7.26-7.30 (m, 2H), 6.90-6.96 (m, 5H), 5.66-5.71 (m, 1H), 4.05 (t, J=6.0 Hz, 2H), 3.40-3.45 (m, 2H), 3.20 (s, 3H), 2.00 (t, J=6.4 Hz, 2H), 1.57 (d, J=6.4 Hz, 3H). LC-MS m/z: 520.0 [M+H]⁺. HPLC Purity (254 nm): 86.27%; t_{R}=9.407 min.

AC2020601-0363

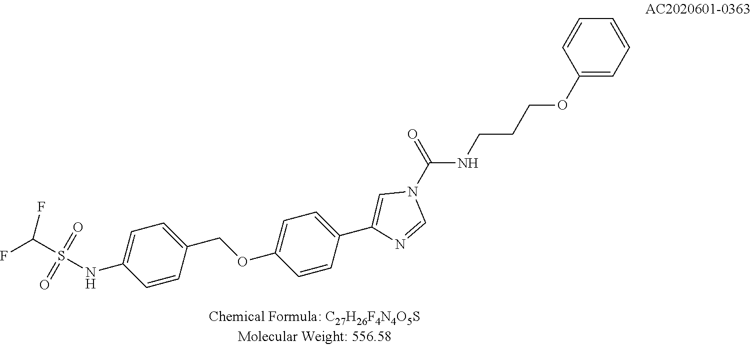 [SMILES: O=C(NCCCOc1ccccc1)n1cnc(-c2ccc(OCc3ccc(NS(=O)(=O)C(F)F)cc3)cc2)c1]

### The Synthesis of tert-butyl 4-(4-((4-(difluoromethylsulfonamido)benzyl)oxy)phenyl)-1H-imidazole-1-carboxylate (363-4)

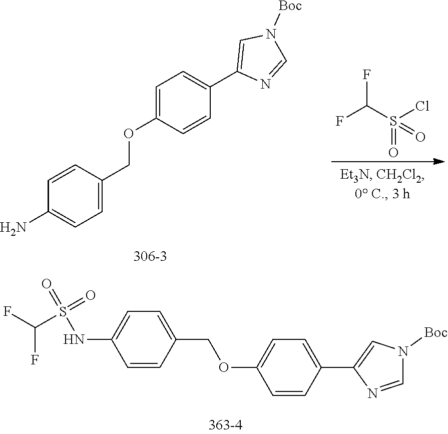 [SMILES: CC(C)(C)OC(=O)n1cnc(-c2ccc(OCc3ccc(N)cc3)cc2)c1 ; O=S(=O)(Cl)C(F)F ; CC(C)(C)OC(=O)n1cnc(-c2ccc(OCc3ccc(NS(=O)(=O)C(F)F)cc3)cc2)c1]

To a suspension of 306-3 (500 mg, 1.37 mmol) in CH₂Cl₂ (10 mL) was added Et₃N (277 mg, 2.74 mmol) and difluoromethanesulfonyl chloride (247 mg, 1.64 mmol) at 0° C. The reaction mixture was stirred at room temperature for 3 hours until the reaction was complete (by LCMS). Water was added and the reaction mixture was extracted with EtOAc (50 mL), the combined organic layer was washed with brine, dried over anhydrous Na₂SO₄ and evaporated under reduced pressure. The crude was purified by silica gel chromatography (5% MeOH/DCM) to afford 363-4 (300 mg, yield: 46%) as a yellow solid.

### The Synthesis of N-(4-((4-(1H-imidazol-4-yl)phenoxy)methyl)phenyl)-1,1-difluoromethanesulfonamide (363-5)

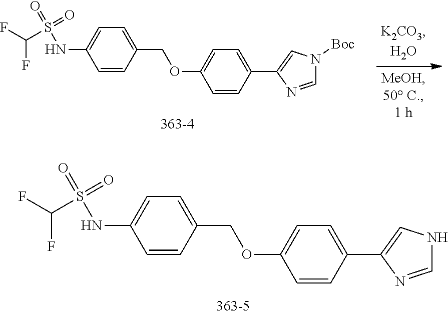 [SMILES: CC(C)(C)OC(=O)n1cnc(-c2ccc(OCc3ccc(NS(=O)(=O)C(F)F)cc3)cc2)c1 ; O=S(=O)(Nc1ccc(COc2ccc(-c3c[nH]cn3)cc2)cc1)C(F)F]

To a stirred solution of 363-4 (300 mg, 0.63 mmol) in MeOH (10 mL) and H₂O (1 mL) was added K₂CO₃ (173 mg, 1.25 mmol). The reaction mixture was stirred at 50° C. for 1 hour until the reaction was complete (by LCMS). The reaction mixture was quenched with water, extracted with EA (20 mL), the combined organic layer was washed with brine, dried over anhydrous Na₂SO₄. The mixture was concentrated under reduced pressure to afford 363-5 (200 mg, yield: 84%) as a white solid, which was used to the next step without purification.

### The Synthesis of 4-(4-((4-(difluoromethylsulfonamido)benzyl)oxy)phenyl)-N-(3-phenoxypropyl)-1H-imidazole-1-carboxamide (AC2020601-0363)

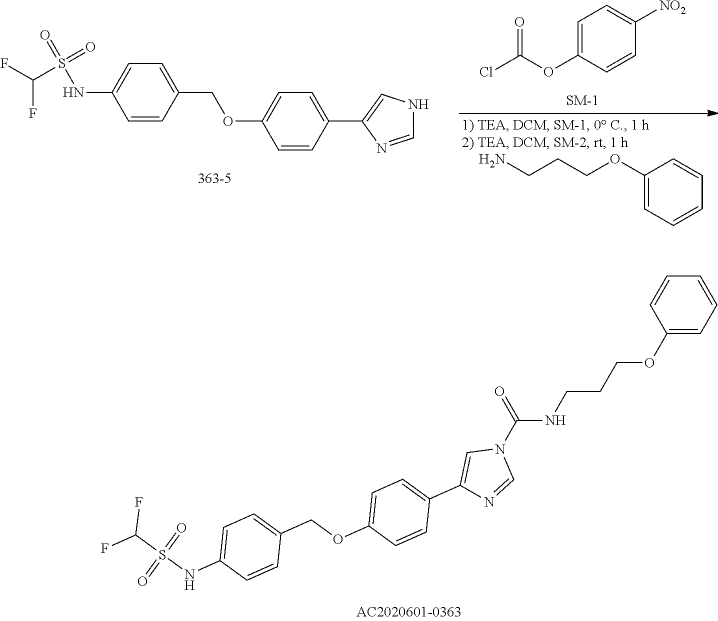 [SMILES: O=S(=O)(Nc1ccc(COc2ccc(-c3c[nH]cn3)cc2)cc1)C(F)F ; O=C(Cl)Oc1ccc([N+](=O)[O-])cc1 ; NCCCOc1ccccc1 ; O=C(NCCCOc1ccccc1)n1cnc(-c2ccc(OCc3ccc(NS(=O)(=O)C(F)F)cc3)cc2)c1]

To a solution of 363-5 (100 mg, 0.26 mmol) in DCM (5 mL) was added 4-nitrophenyl carbonochloridate (64 mg, 0.32 mmol) and Et₃N (5 mL) at 0° C. and stirred for 1 hour. Then the reaction mixture was allowed to warm to room temperature and 3-phenoxypropan-1-amine (48 mg, 0.32 mmol) was added. After 1 hour stirring, the reaction mixture was quenched with water, extracted with DCM (20 mL), the combined organic layer was washed with brine, dried over anhydrous Na₂SO₄ and evaporated under reduced pressure. The crude was purified by silica gel chromatography (5% MeOH/DCM) to afford AC2020601-0363 (6 mg, yield: 4%) as a white solid. ¹H NMR (400 MHz, DMSO-d₆) δ 11.06 (br, 1H), 8.60 (t, J=5.6 Hz, 1H), 8.27 (s, 1H), 8.01 (s, 1H), 7.72 (d, J=8.4 Hz, 2H), 7.43 (d, J=8.4 Hz, 2H), 7.23-7.30 (m, 5H), 7.03 (d, J=8.8 Hz, 2H), 6.91-6.98 (m, 3H), 5.07 (s, 2H), 4.06 (t, J=6.0 Hz, 2H), 3.42-3.47 (m, 2H), 2.00-2.03 (m, 2H). LC-MS m/z: 556.8 [M+H]⁺. HPLC Purity (254 nm): 92.31%; t_{R}=7.921 min.

AC2020601-0384a

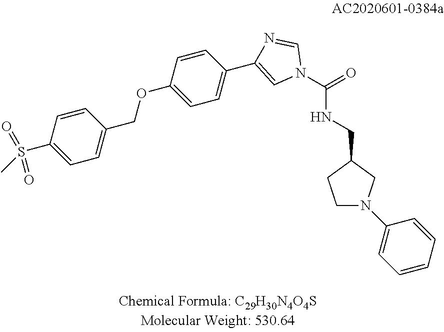 [SMILES: CS(=O)(=O)c1ccc(COc2ccc(-c3cn(C(=O)NC[C@@H]4CCN(c5ccccc5)C4)cn3)cc2)cc1]

### The Synthesis of (S)-tert-butyl ((1-phenylpyrrolidin-3-yl)methyl)carbamate (384a-2)

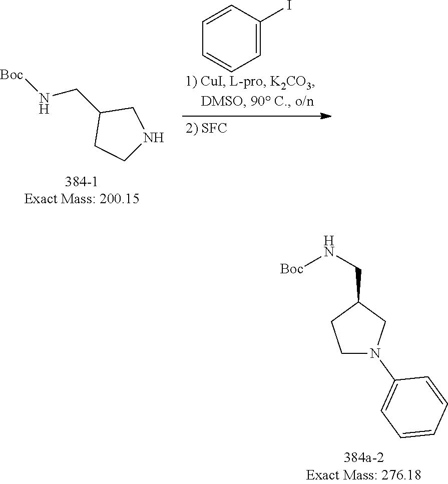 [SMILES: Ic1ccccc1 ; CC(C)(C)OC(=O)NCC1CCNC1 ; CC(C)(C)OC(=O)NC[C@@H]1CCN(c2ccccc2)C1]

A mixture of 384-1 (2.15 g, 10.74 mmol), iodobenzene (4.38 g, 21.48 mmol), CuI (204 mg, 1.07 mmol), L-pro (124 mg, 1.07 mmol) and K₂CO₃ (2.97 g, 21.48 mmol) in DMSO (10 mL) was stirred at 90° C. overnight under N₂. The reaction mixture was quenched with water and extracted with EtOAc (50 mL), the combined organic layer was washed with brine, dried over anhydrous Na₂SO₄ and evaporated under reduced pressure. The crude was purified by silica gel chromatography (25% EA/PE) to afford the racemate 384a-2 (2.4 g, yield 77%).

250 mg racemate was subjected to SFC separation to afford enantiomer 384a-2 (120 mg) as yellow oil. Note: The absolute configuration is tentatively assigned.

### The Synthesis of (S)-(1-phenylpyrrolidin-3-yl)methanamine (384a-3)

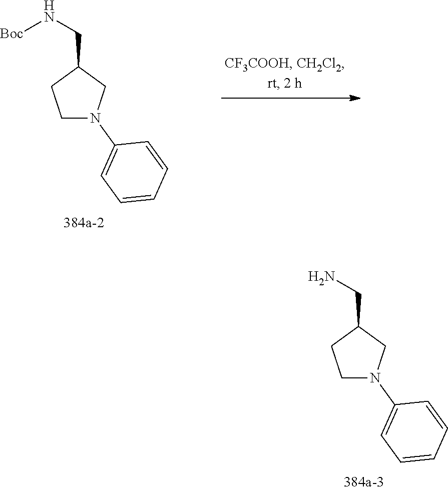 [SMILES: CC(C)(C)OC(=O)NC[C@@H]1CCN(c2ccccc2)C1 ; NC[C@@H]1CCN(c2ccccc2)C1]

To a stirred solution of 384a-2 (120 mg, 0.43 mmol) in CH₂Cl₂ (3 mL) was added TFA (3 mL). The reaction mixture was stirred at room temperature for 2 hours until the reaction was complete (by LCMS). The mixture was concentrated under reduced pressure to afford 384a-3 (75 mg, yield: 98%) as yellow oil, which was used to the next step without purification.

### The Synthesis of (S)-4-(4-((4-(methylsulfonyl)benzyl)oxy)phenyl)-N-((1-phenylpyrrolidin-3-yl)methyl)-1H-imidazole-1-carboxamide (AC2020601-0384a)

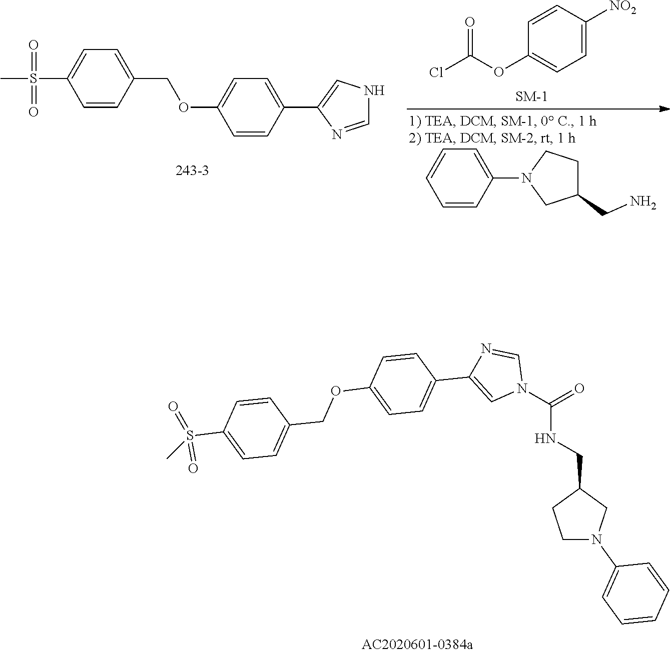 [SMILES: CS(=O)(=O)c1ccc(COc2ccc(-c3c[nH]cn3)cc2)cc1 ; O=C(Cl)Oc1ccc([N+](=O)[O-])cc1 ; NC[C@@H]1CCN(c2ccccc2)C1 ; CS(=O)(=O)c1ccc(COc2ccc(-c3cn(C(=O)NC[C@@H]4CCN(c5ccccc5)C4)cn3)cc2)cc1]

To a solution of 243-3 (153 mg, 0.47 mmol) in DCM (5 mL) was added 4-nitrophenyl carbonochloridate (94 mg, 0.47 mmol) and Et₃N (5 mL) at 0° C. and stirred for 1 hour. Then the reaction mixture was allowed to warm to room temperature and (S)-(1-phenylpyrrolidin-3-yl)methanamine (75 mg, 0.43 mmol) was added. After 1 hour stirring, the reaction mixture was quenched with water, extracted with DCM (20 mL), the combined organic layer was washed with brine, dried over anhydrous Na₂SO₄ and evaporated under reduced pressure. The crude was purified by silica gel chromatography (5% MeOH/DCM) to afford AC2020601-0384a (96 mg, yield: 43%) as a white solid. ¹H NMR (400 MHz, DMSO-d₆) δ 8.65 (t, J=5.2 Hz, 1H), 8.30 (s, 1H), 8.04 (s, 1H), 7.96 (d, J=8.0 Hz, 2H), 7.72-7.76 (m, 4H), 7.13-7.17 (m, 2H), 7.07 (d, J=8.4 Hz, 2H), 6.57-6.60 (m, 1H), 6.52-6.54 (m, 2H), 5.28 (s, 2H), 3.31-3.40 (m, 4H), 3.22-3.27 (m, 4H), 3.05-3.09 (m, 1H), 2.59-2.66 (m, 1H), 2.09-2.15 (m, 1H), 1.78-1.85 (m, 1H). LC-MS m/z: 553.2 [M+Na]⁺. HPLC Purity (254 nm): 100%; t_{R}=9.484 min. Note: The absolute configuration is tentatively assigned.

AC2020601-0384b

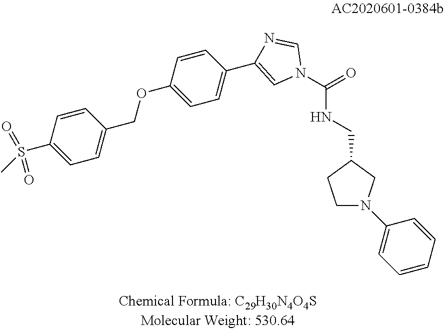 [SMILES: CS(=O)(=O)c1ccc(COc2ccc(-c3cn(C(=O)NC[C@H]4CCN(c5ccccc5)C4)cn3)cc2)cc1]

### The Synthesis of (R)-tert-butyl ((1-phenylpyrrolidin-3-yl)methyl)carbamate (384b-2)

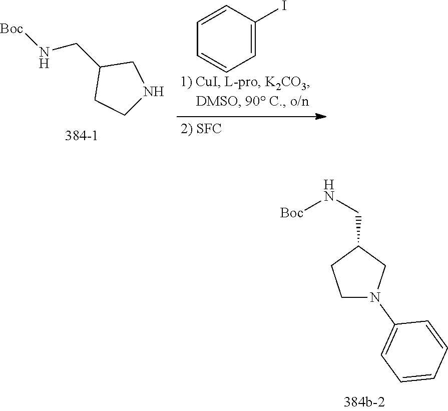 [SMILES: CC(C)(C)OC(=O)NCC1CCNC1 ; Ic1ccccc1 ; CC(C)(C)OC(=O)NC[C@H]1CCN(c2ccccc2)C1]

A mixture of 384-1 (2.15 g, 10.74 mmol), iodobenzene (4.38 g, 21.48 mmol), CuI (204 mg, 1.07 mmol), L-pro (124 mg, 1.07 mmol) and K₂CO₃ (2.97 g, 21.48 mmol) in DMSO (10 mL) was stirred at 90° C. overnight under N₂. The reaction mixture was quenched with water and extracted with EtOAc (50 mL), the combined organic layer was washed with brine, dried over anhydrous Na₂SO₄ and evaporated under reduced pressure. The crude was purified by silica gel chromatography (25% EA/PE) to afford the racemate 384b-2 (2.4 g, yield 77%).

250 mg racemate was subjected to SFC separation to afford enantiomer 384b-2 (120 mg) as yellow oil. Note: The absolute configuration is tentatively assigned.

### The Synthesis of (R)-(1-phenylpyrrolidin-3-yl)methanamine (384b-3)

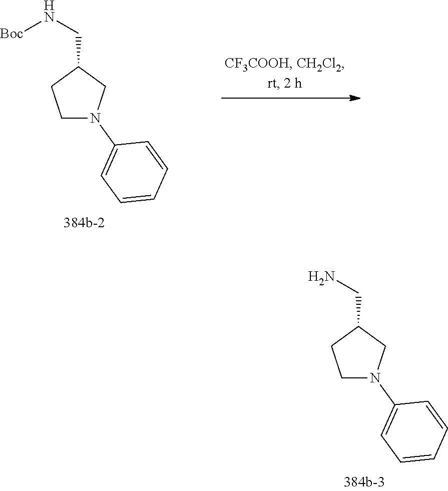 [SMILES: CC(C)(C)OC(=O)NC[C@H]1CCN(c2ccccc2)C1 ; NC[C@H]1CCN(c2ccccc2)C1]

To a stirred solution of 384b-2 (120 mg, 0.43 mmol) in CH₂Cl₂ (3 mL) was added TFA (3 mL). The reaction mixture was stirred at room temperature for 2 hours until the reaction was complete (by LCMS). The mixture was concentrated under reduced pressure to afford 384b-3 (75 mg, yield: 98%) as yellow oil, which was used to the next step without purification.

### The Synthesis of (R)-4-(4-((4-(methylsulfonyl)benzyl)oxy)phenyl)-N-((1-phenylpyrrolidin-3-yl)methyl)-1H-imidazole-1-carboxamide (AC2020601-0384b)

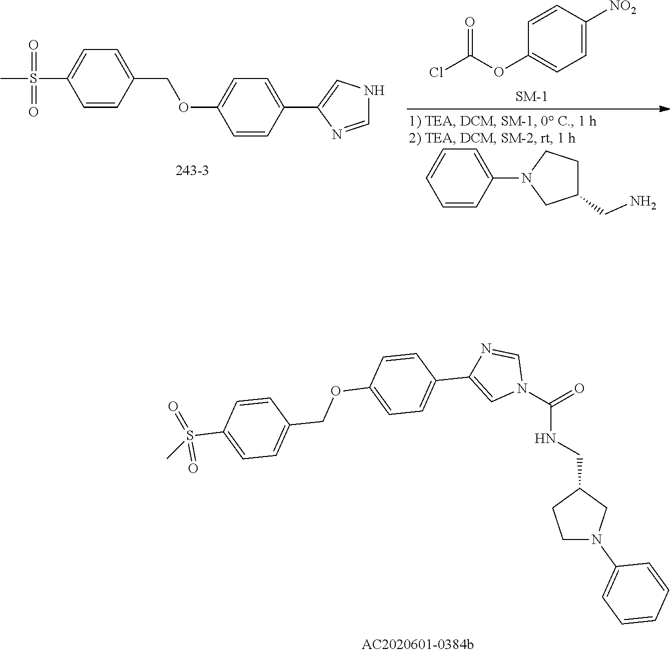 [SMILES: CS(=O)(=O)c1ccc(COc2ccc(-c3c[nH]cn3)cc2)cc1 ; O=C(Cl)Oc1ccc([N+](=O)[O-])cc1 ; NC[C@H]1CCN(c2ccccc2)C1 ; CS(=O)(=O)c1ccc(COc2ccc(-c3cn(C(=O)NC[C@H]4CCN(c5ccccc5)C4)cn3)cc2)cc1]

To a solution of 243-3 (153 mg, 0.47 mmol) in DCM (5 mL) was added 4-nitrophenyl carbonochloridate (94 mg, 0.47 mmol) and Et₃N (5 mL) at 0° C. and stirred for 1 hour. Then the reaction mixture was allowed to warm to room temperature and (R)-(1-phenylpyrrolidin-3-yl)methanamine (75 mg, 0.43 mmol) was added. After 1 hour stirring, the reaction mixture was quenched with water, extracted with DCM (20 mL), the combined organic layer was washed with brine, dried over anhydrous Na₂SO₄ and evaporated under reduced pressure. The crude was purified by silica gel chromatography (5% MeOH/DCM) to afford AC2020601-0384b (108 mg, yield: 48%) as a white solid. ¹H NMR (400 MHz, DMSO-d₆) δ 8.65 (t, J=5.2 Hz, 1H), 8.30 (s, 1H), 8.04 (s, 1H), 7.96 (d, J=8.4 Hz, 2H), 7.72-7.76 (m, 4H), 7.13-7.17 (m, 2H), 7.07 (d, J=8.4 Hz, 2H), 6.57-6.60 (m, 1H), 6.52-6.54 (m, 2H), 5.28 (s, 2H), 3.31-3.40 (m, 4H), 3.21-3.26 (m, 4H), 3.05-3.09 (m, 1H), 2.59-2.66 (m, 1H), 2.09-2.16 (m, 1H), 1.76-1.85 (m, 1H). LC-MS m/z: 553.2 [M+Na]⁺. HPLC Purity (254 nm): 100%; t_{R}=9.480 min. Note: The absolute configuration is tentatively assigned.

### Example 2: Additional Experimental Procedures

AC2020601-0373

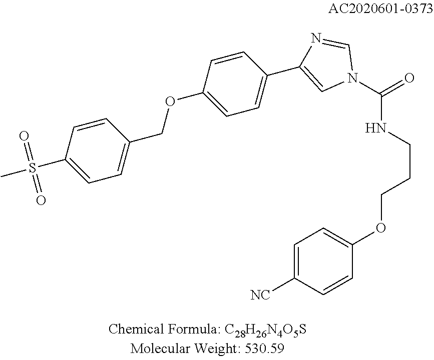 [SMILES: CS(=O)(=O)c1ccc(COc2ccc(-c3cn(C(=O)NCCCOc4ccc(C#N)cc4)cn3)cc2)cc1]

### The Synthesis of tert-butyl 3-(4-cyanophenoxy)propylcarbamate (373-1-2)

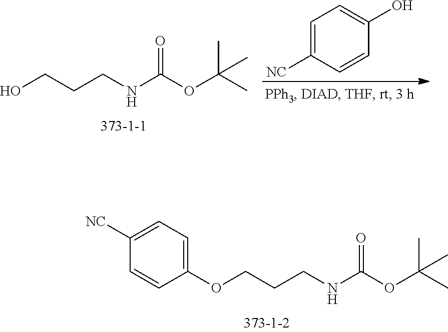

To a suspension of 373-1-1 (350 mg, 2.0 mmol) in THF (10 mL) was added 4-hydroxybenzonitrile (286 mg, 2.4 mmol), PPh₃ (786 mg, 3.0 mmol) and DIAD (525 mg, 2.6 mmol). The reaction mixture was stirred at room temperature for 3 hours until the reaction was complete (by LCMS). Water was added and the reaction mixture was extracted with EtOAc (50 mL), the combined organic layer was washed with brine, dried over anhydrous Na₂SO₄ and evaporated under reduced pressure. The crude was purified by silica gel chromatography (30% EA/PE) to afford 373-1-2 (410 mg, yield: 74.3%) as white oil.

### The Synthesis of 4-(3-aminopropoxy)benzonitrile (373-1-3)

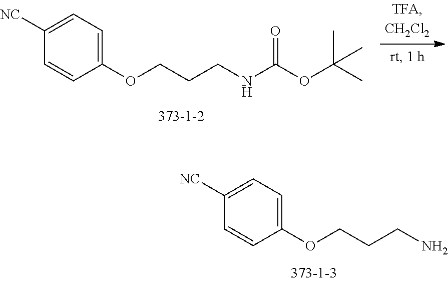 [SMILES: CC(C)(C)OC(=O)NCCCOc1ccc(C#N)cc1 ; N#Cc1ccc(OCCCN)cc1]

To a stirred solution of 373-1-2 (410 mg, 1.5 mmol) in CH₂Cl₂ (10 mL) was added TFA (10 mL). The reaction mixture was stirred at room temperature for 1 hour until the reaction was complete (by LCMS). The reaction mixture was quenched with water, extracted with DCM (20 mL), the combined organic layer was washed with brine, dried over anhydrous Na₂SO₄. The mixture was concentrated under reduced pressure to afford 373-1-3 (230 mg, yield: 88%) as yellow oil, which was used to the next step without purification.

### The Synthesis of N-(3-(4-cyanophenoxy)propyl)-4-(4-(4-(methylsulfonyl)benzyloxy)phenyl)-1H-imidazole-1-carboxamide (AC2020601-0373)

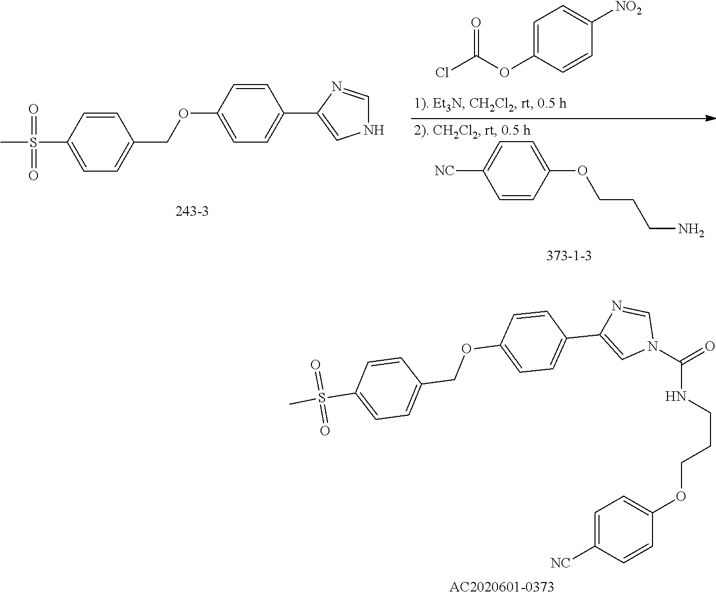 [SMILES: O=C(Cl)Oc1ccc([N+](=O)[O-])cc1 ; CS(=O)(=O)c1ccc(COc2ccc(-c3c[nH]cn3)cc2)cc1 ; N#Cc1ccc(OCCCN)cc1 ; CS(=O)(=O)c1ccc(COc2ccc(-c3cn(C(=O)NCCCOc4ccc(C#N)cc4)cn3)cc2)cc1]

To a solution of 243-3 (329 mg, 1.0 mmol) in DCM (5 mL) was added 4-nitrophenyl carbonochloridate (302 mg, 1.5 mmol) and Et₃N (5 mL) at room temperature and stirred for 0.5 hour. Then 4-(3-aminopropoxy)benzonitrile (373-1-3, 352 mg, 2.0 mmol) was added. After 0.5 hour stirring, the reaction mixture was quenched with water, extracted with DCM (20 mL), the combined organic layer was washed with brine, dried over anhydrous Na₂SO₄ and evaporated under reduced pressure. The crude was purified by silica gel chromatography (5% MeOH/DCM) to afford AC2020601-0373 (28 mg, yield: 5%) as a white solid. ¹H NMR (400 MHz, DMSO-d₆) δ 8.58 (t, J=5.6 Hz, 1H), 8.26 (d, J=1.2 Hz, 1H), 8.00 (d, J=0.8 Hz, 1H), 7.95 (d, J=8.4 Hz, 2H), 7.71-7.78 (m, 6H), 7.11 (d, J=8.8 Hz, 2H), 7.06 (d, J=8.8 Hz, 2H), 5.28 (s, 2H), 4.17 (t, J=6.0 Hz, 2H), 3.44 (dd, J=12.0 Hz, J=6.4 Hz, 2H), 3.22 (s, 3H), 2.02-2.08 (m, 2H). LC-MS m/z: 531.0 [M+H]⁺. HPLC Purity (254 nm): 94.92%; t_{R}=8.733 min.

AC2020601-0374

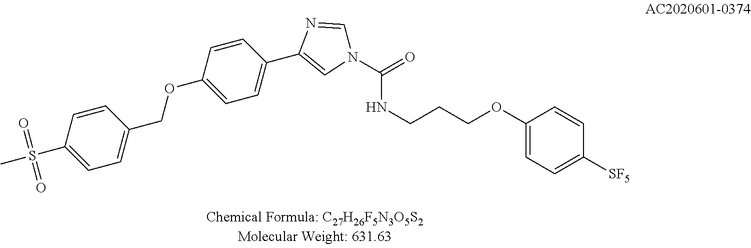

The synthesis of (374-2)

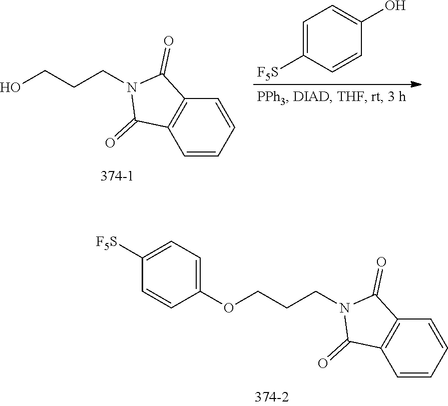

To a suspension of 374-1 (2.05 g, 10.0 mmol) in THF (30 mL) was added SM (2.20 g, 10.0 mmol), PPh₃ (2.88 g, 11.0 mmol) and DIAD (2.22 g, 11.0 mmol). The reaction mixture was stirred at room temperature for 3 hours until the reaction was complete (by LCMS). Saturated NaCl solution was added and the reaction mixture was extracted with EtOAc (20 mL), the combined organic layer was washed with brine, dried over anhydrous Na₂SO₄ and evaporated under reduced pressure. The crude was purified by silica gel chromatography (40% EA/PE) to afford 374-2 (3.10 g, yield: 76%) as yellow oil.

### The Synthesis of (374-3)

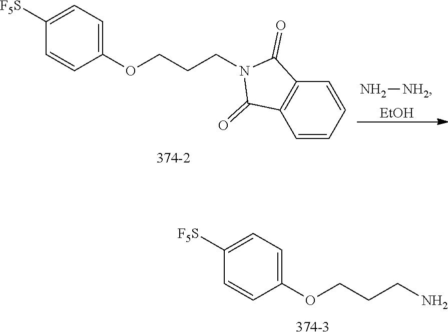

To a stirred solution of 374-2 (407 mg, 1.00 mmol) in EtOH (10 mL) was added NH₂—NH₂·H₂O (1 mL). The reaction mixture was stirred at 80° C. for 1 hour until the reaction was complete (by LCMS). The mixture was filtered and concentrated under reduced pressure to afford 374-3 (270 mg, yield: 97%) as yellow oil, which was used to the next step without purification.

### The Synthesis of (AC2020601-0374)

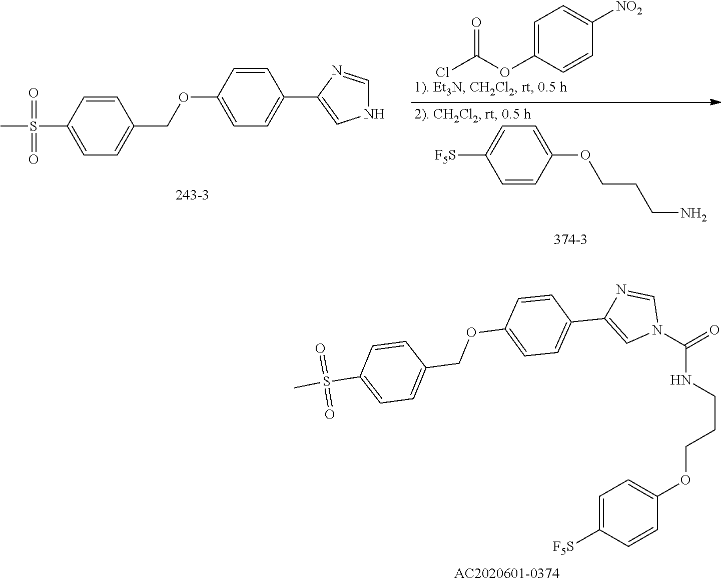

To a solution of 243-3 (170 mg, 0.52 mmol) in DCM (10 mL) was added SM1 (115 mg, 0.57 mmol) and Et₃N (1 mL) at 0° C. and stirred for 1 hour. Then the reaction mixture was allowed to warm to room temperature and SM2 (144 mg, 0.52 mmol) was added. After 1 hour stirring, the reaction mixture was quenched with water, extracted with DCM (20 mL), the combined organic layer was washed with brine, dried over anhydrous Na₂SO₄ and evaporated under reduced pressure. The crude was purified by silica gel chromatography (10% MeOH/DCM) to afford AC2020601-0374 (161 mg, yield: 49%) as a white solid. ¹H NMR (400 MHz, DMSO-d₆) δ 8.59 (t, J=5.6 Hz, 1H), 8.27 (d, J=1.6 Hz, 1H), 8.00 (d, J=0.8 Hz, 1H), 7.96 (d, J=8.4 Hz, 2H), 7.82 (d, J=9.2 Hz, 2H), 7.71-7.75 (m, 4H), 7.06-7.12 (m, 4H), 5.28 (s, 2H), 4.17 (t, J=5.6 Hz, 2H), 3.42-3.47 (m, 2H), 3.22 (s, 3H), 2.03-2.06 (m, 2H). LC-MS m/z: 631.9 [M+H]⁺. HPLC Purity (254 nm): 95.18%; t_{R}=9.838 min.

AC2020601-0377

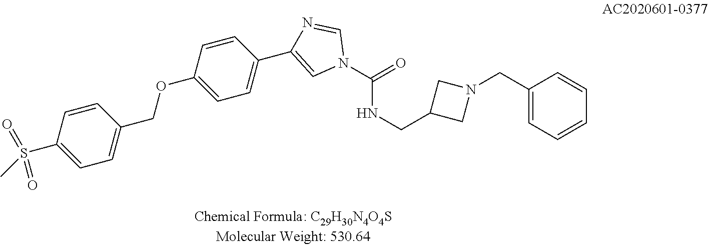 [SMILES: CS(=O)(=O)c1ccc(COc2ccc(-c3cn(C(=O)NCC4CN(Cc5ccccc5)C4)cn3)cc2)cc1]

### The Synthesis of tert- butyl ((1-benzylazetidin-3-yl)methyl)carbamate (377-2)

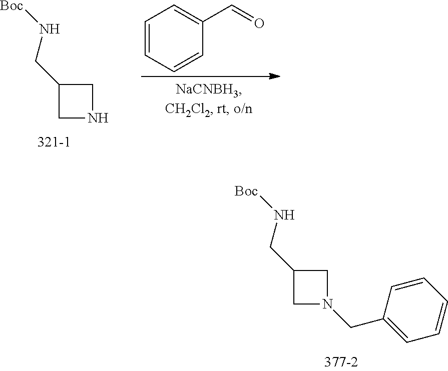 [SMILES: CC(C)(C)OC(=O)NCC1CNC1 ; O=Cc1ccccc1 ; CC(C)(C)OC(=O)NCC1CN(Cc2ccccc2)C1]

To a stirred solution of 321-1 (1.86 g, 9.99 mmol) in CH₂Cl₂ (10 mL) was added benzaldehyde (2.12 g, 19.97 mmol) and NaCNBH₃ (1.26 g, 19.97 mmol). The reaction mixture was stirred at room temperature for 16 hours until the reaction was complete (by LCMS). The reaction mixture was quenched with water and extracted with CH₂Cl₂ (50 mL), the combined organic layer was washed with brine, dried over anhydrous Na₂SO₄ and evaporated under reduced pressure. The crude was purified by silica gel chromatography (40% EA/PE) to afford 377-2 (2.56 g, yield: 93%) as yellow oil.

### The Synthesis of (1-benzylazetidin-3-yl)methanamine (377-3)

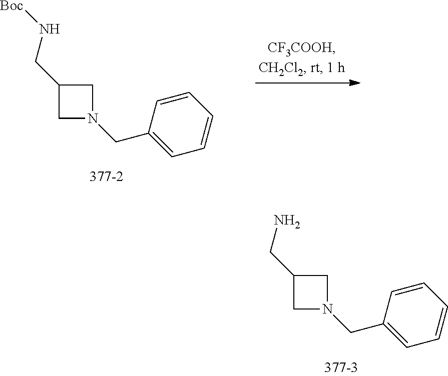 [SMILES: CC(C)(C)OC(=O)NCC1CN(Cc2ccccc2)C1 ; NCC1CN(Cc2ccccc2)C1]

To a stirred solution of 377-2 (2.00 g, 7.24 mmol) in CH₂Cl₂ (10 mL) was added TFA (5 mL). The reaction mixture was stirred at room temperature for 1 hour until the reaction was complete (by LCMS). The mixture was concentrated under reduced pressure to afford 377-3 (1.18 g, crude) as yellow oil, which was used to the next step without purification.

### The Synthesis of N-((1-benzylazetidin-3-yl)methyl)-4-(4-((4-(methylsulfonyl)benzyl)oxy)phenyl)-1H-imidazole-1-carboxamide (AC2020601-0377)

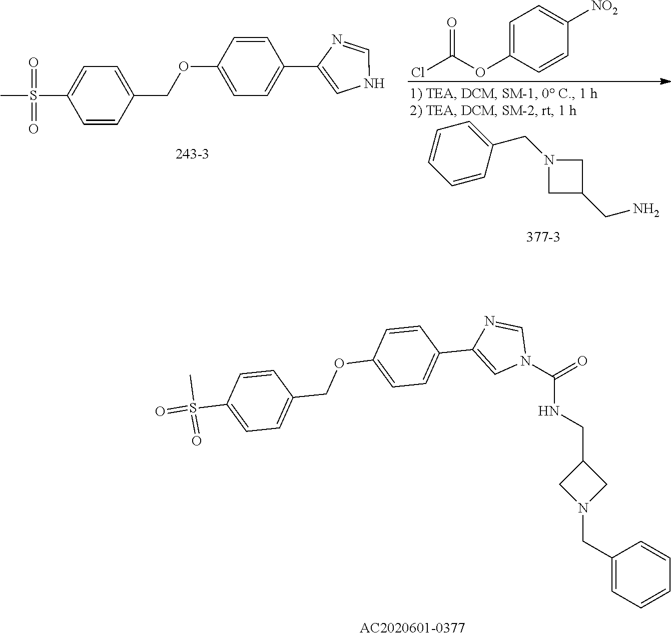 [SMILES: CS(=O)(=O)c1ccc(COc2ccc(-c3c[nH]cn3)cc2)cc1 ; O=C(Cl)Oc1ccc([N+](=O)[O-])cc1 ; NCC1CN(Cc2ccccc2)C1 ; CS(=O)(=O)c1ccc(COc2ccc(-c3cn(C(=O)NCC4CN(Cc5ccccc5)C4)cn3)cc2)cc1]

To a solution of 243-3 (328 mg, 0.99 mmol) in DCM (5 mL) was added 4-nitrophenyl carbonochloridate (221 mg, 1.10 mmol) and Et₃N (2 mL) at 0° C. and stirred for 1 hour. Then the reaction mixture was allowed to warm to room temperature and 377-3 (176 mg, 1.10 mmol) was added. After 1 hour stirring, the reaction mixture was quenched with water, extracted with DCM (20 mL), the combined organic layer was washed with brine, dried over anhydrous Na₂SO₄ and evaporated under reduced pressure. The crude was purified by Prep-HPLC to afford AC2020601-0377 (185 mg, yield: 34.9%) as a white solid. ¹H NMR (400 MHz, DMSO-d₆) δ 8.66 (s, 1H), 8.29 (s, 1H), 8.05 (s, 1H), 7.96 (d, J=8.4 Hz, 2H), 7.71-7.74 (m, 4H), 7.20-7.31 (m, 5H), 7.06 (d, J=8.8 Hz, 2H), 5.28 (s, 2H), 3.53 (s, 2H), 3.46 (t, J=6.4 Hz, 2H), 3.21-3.24 (m, 5H), 2.95 (t, J=5.6 Hz, 2H), 2.59-2.63 (m, 1H). LC-MS m/z: 531.2 [M+H]⁺. HPLC Purity (254 nm): 94.64%; t_{R}=8.534 min.

AC2020601-0379

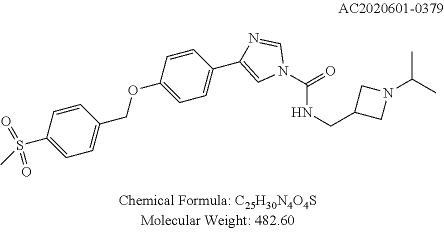 [SMILES: CC(C)N1CC(CNC(=O)n2cnc(-c3ccc(OCc4ccc(S(C)(=O)=O)cc4)cc3)c2)C1]

### The Synthesis of tert -butyl ((1-isopropylazetidin-3-yl)methyl)carbamate (379-2)

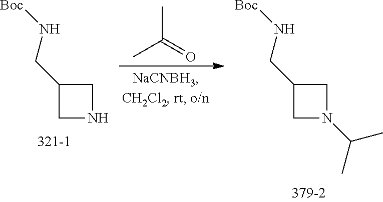

To a stirred solution of 321-1 (1.86 g, 9.99 mmol) in CH₂Cl₂ (10 mL) was added propan-2-one (1.16 g, 19.97 mmol) and NaCNBH₃ (1.26 g, 19.97 mmol). The reaction mixture was stirred at room temperature for 12 hours until the reaction was complete (by LCMS). The reaction mixture was quenched with water and extracted with CH₂Cl₂ (50 mL), the combined organic layer was washed with brine, dried over anhydrous Na₂SO₄ and evaporated under reduced pressure. The crude was purified by silica gel chromatography (40% EA/PE) to afford 379-2 (2.21 g, yield: 96.9%) as yellow oil.

### The Synthesis of (1-isopropylazetidin-3-yl)methanamine (379-3)

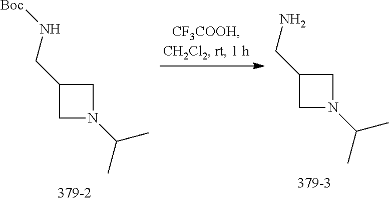

To a stirred solution of 379-2 (2.00 g, 8.76 mmol) in CH₂Cl₂ (10 mL) was added TFA (5 mL). The reaction mixture was stirred at room temperature for 1 hour until the reaction was complete (by LCMS). The mixture was concentrated under reduced pressure to afford 379-3 (1.08 g, crude) as yellow oil, which was used to the next step without purification.

### The Synthesis of N -((1-isopropylazetidin-3-yl)methyl)-4-(4-((4-(methylsulfonyl)benzyl)oxy)phenyl)-1H-imidazole-1-carboxamide (AC2020601-0379)

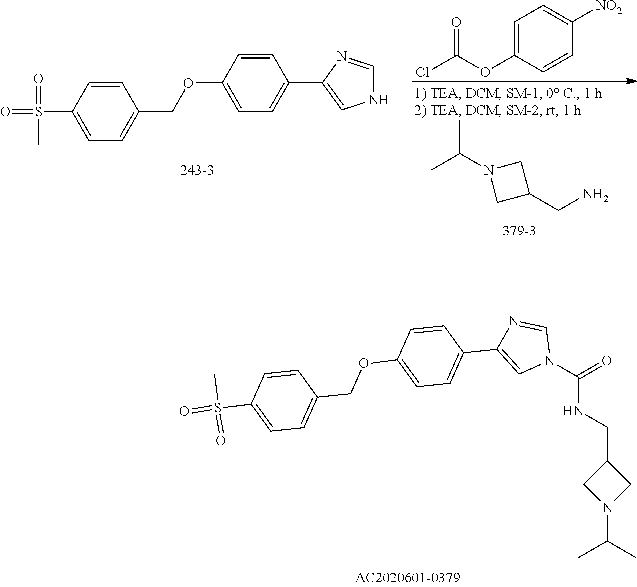

To a solution of 243-3 (328 mg, 0.99 mmol) in DCM (5 mL) was added 4-nitrophenyl carbonochloridate (221 mg, 1.10 mmol) and Et₃N (2 mL) at 0° C. and stirred for 1 hour. Then the reaction mixture was allowed to warm to room temperature and 377-3 (128 mg, 0.99 mmol) was added. After 1 hour stirring, the reaction mixture was quenched with water, extracted with DCM (20 mL), the combined organic layer was washed with brine, dried over anhydrous Na₂SO₄ and evaporated under reduced pressure. The crude was purified by Prep-HPLC to afford AC2020601-0379 (74 mg, yield: 15.4%) as a white solid. ¹H NMR (400 MHz, DMSO-d₆) δ 8.57 (t, J=5.6 Hz, 1H), 8.26 (s, 1H), 8.01 (s, 1H), 7.96 (d, J=8.4 Hz, 2H), 7.71-7.75 (m, 4H), 7.07 (d, J=9.2 Hz, 2H), 5.28 (s, 2H), 3.41-3.44 (m, 2H), 3.22 (s, 3H), 3.13-3.17 (m, 2H), 2.83-2.86 (m, 2H), 2.45-2.47 (m, 1H), 2.19-2.22 (m, 1H), 0.82 (d, J=6.0 Hz, 6H). LC-MS m/z: 483.2 [M+H]⁺. HPLC Purity (254 nm): 93.04%; t_{R}=633.1 min.

AC2020601-0380

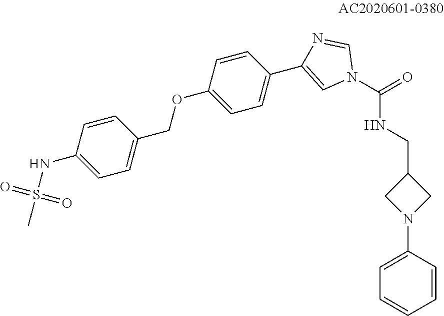 [SMILES: CS(=O)(=O)Nc1ccc(COc2ccc(-c3cn(C(=O)NCC4CN(c5ccccc5)C4)cn3)cc2)cc1]

### The Synthesis of 4-(4-((4-(methylsulfonamido)benzyl)oxy)phenyl)-N-((1-phenylazetidin-3-yl)methyl)-1H-imidazole-1-carboxamide (AC2020601-0380)

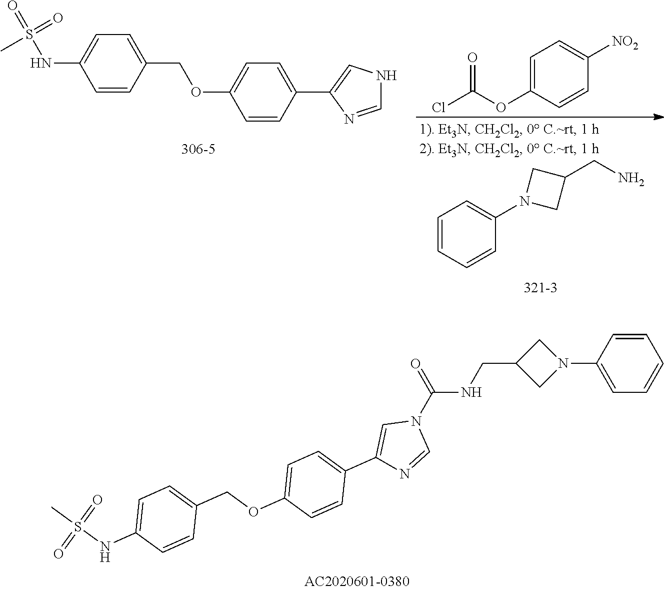 [SMILES: CS(=O)(=O)Nc1ccc(COc2ccc(-c3c[nH]cn3)cc2)cc1 ; O=C(Cl)Oc1ccc([N+](=O)[O-])cc1 ; NCC1CN(c2ccccc2)C1 ; CS(=O)(=O)Nc1ccc(COc2ccc(-c3cn(C(=O)NCC4CN(c5ccccc5)C4)cn3)cc2)cc1]

To a solution of 306-5 (100 mg, 0.29 mmol) in DCM (5 mL) was added 4-nitrophenyl carbonochloridate (70 mg, 0.35 mmol) and Et₃N (2 mL) at 0° C. and stirred for 1 hour. Then the reaction mixture was allowed to warm to room temperature and 321-3 (47 mg, 0.35 mmol) was added. After 1 hour stirring, the reaction mixture was quenched with water, extracted with DCM (20 mL), the combined organic layer was washed with brine, dried over anhydrous Na₂SO₄ and evaporated under reduced pressure. The crude was purified by silica gel chromatography (5% MeOH/DCM) to afford AC2020601-0380 (3 mg, yield: 1.9%) as a white solid. ¹H NMR (400 MHz, DMSO-d₆) δ 8.54 (s, 1H), 8.18 (s, 1H), 8.27 (s, 1H), 8.03 (s, 1H), 7.77 (d, J=8.8 Hz, 2H), 7.41 (d, J=8.8 Hz, 2H), 7.31 (d, J=8.4 Hz, 2H), 7.13-7.17 (m, 2H), 7.04 (d, J=8.8 Hz, 2H), 6.63-6.67 (m, 1H), 6.36-6.42 (m, 3H), 5.01 (s, 2H), 3.81-3.85 (m, 2H), 3.68 (s, 3H), 3.49-3.54 (m, 2H), 3.31-3.38 (m, 2H), 2.80-2.84 (m, 1H). LC-MS m/z: 532.0 [M+H]⁺. HPLC Purity (254 nm): 100%; t_{R}=8.498 min.

AC2020601-0382

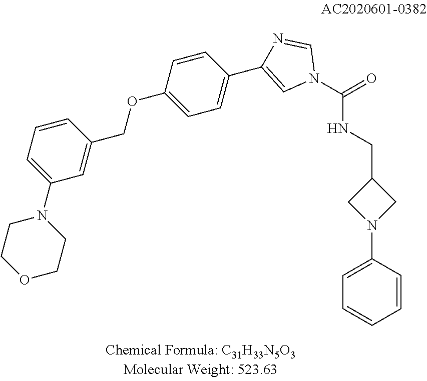 [SMILES: O=C(NCC1CN(c2ccccc2)C1)n1cnc(-c2ccc(OCc3cccc(N4CCOCC4)c3)cc2)c1]

### The Synthesis of tert-butyl 4-(4-(3-morpholinobenzyloxy)phenyl)-1H-imidazole-1-carboxylate (382-2)

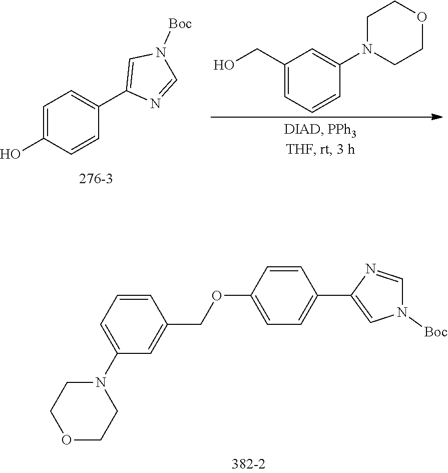 [SMILES: CC(C)(C)OC(=O)n1cnc(-c2ccc(O)cc2)c1 ; OCc1cccc(N2CCOCC2)c1 ; CC(C)(C)OC(=O)n1cnc(-c2ccc(OCc3cccc(N4CCOCC4)c3)cc2)c1]

To a suspension of 276-3 (520 mg, 2.0 mmol) in THF (10 mL) was added (3-morpholinophenyl)methanol (386 mg, 2.0 mmol), PPh₃ (629 mg, 2.4 mmol) and DIAD (525 mg, 2.6 mmol). The reaction mixture was stirred at room temperature for 3 hours until the reaction was complete (by LCMS). Water was added and the reaction mixture was extracted with EtOAc (50 mL), the combined organic layer was washed with brine, dried over anhydrous Na₂SO₄ and evaporated under reduced pressure. The crude was purified by silica gel chromatography (30% EA/PE) to afford 382-2 (450 mg, yield: 51.7%) as brown oil.

### The Synthesis of 4-(3-((4-(1H-imidazol-4-yl)phenoxy)methyl)phenyl)morpholine (382-3)

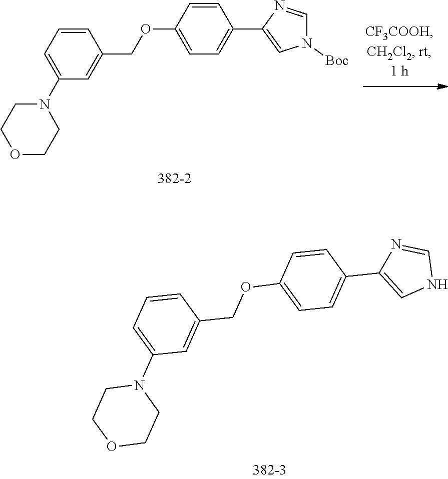 [SMILES: CC(C)(C)OC(=O)n1cnc(-c2ccc(OCc3cccc(N4CCOCC4)c3)cc2)c1 ; c1cc(COc2ccc(-c3c[nH]cn3)cc2)cc(N2CCOCC2)c1]

To a stirred solution of 382-2 (450 mg, 1.0 mmol) in CH₂Cl₂ (10 mL) was added TFA (10 mL). The reaction mixture was stirred at room temperature for 1 hour until the reaction was complete (by LCMS). The reaction mixture was quenched with water, extracted with DCM (20 mL), the combined organic layer was washed with brine, dried over anhydrous Na₂SO₄. The mixture was concentrated under reduced pressure to afford 382-3 (300 mg, yield: 88%) as yellow oil, which was used to the next step without purification.

### The Synthesis of 4-(4-(3-morpholinobenzyloxy)phenyl)-N-((1-phenylazetidin-3-yl)methyl)-1H-imidazole-1-carboxamide (AC2020601-0382)

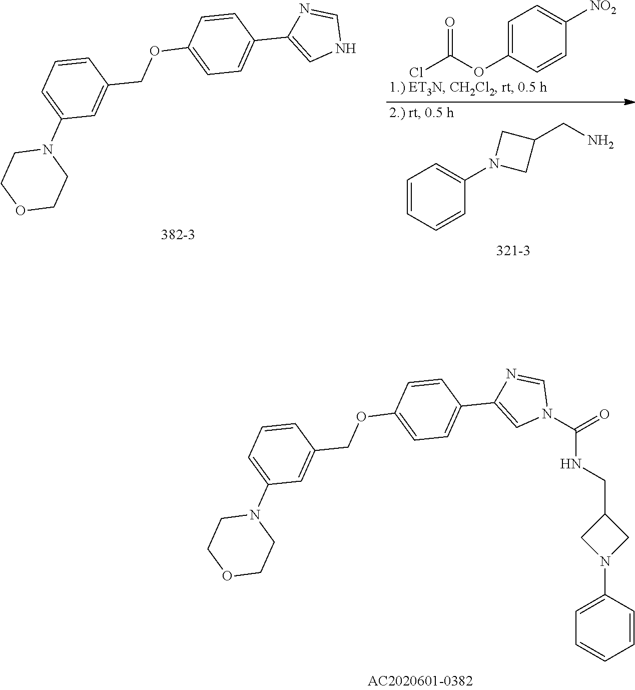 [SMILES: c1cc(COc2ccc(-c3c[nH]cn3)cc2)cc(N2CCOCC2)c1 ; NCC1CN(c2ccccc2)C1 ; O=C(Cl)Oc1ccc([N+](=O)[O-])cc1 ; O=C(NCC1CN(c2ccccc2)C1)n1cnc(-c2ccc(OCc3cccc(N4CCOCC4)c3)cc2)c1]

To a solution of 382-3 (300 mg, 0.9 mmol) in DCM (5 mL) was added 4-nitrophenyl carbonochloridate (271 mg, 1.35 mmol) and Et₃N (5 mL) at room temperature and stirred for 0.5 hour. Then (1-phenylazetidin-3-yl)methanamine (321-3, 292 mg, 1.8 mmol) was added. After 0.5 hour stirring, the reaction mixture was quenched with water, extracted with DCM (20 mL), the combined organic layer was washed with brine, dried over anhydrous Na₂SO₄ and evaporated under reduced pressure. The crude was purified by silica gel chromatography (5% MeOH/DCM) to afford AC2020601-0382 (12 mg, yield: 2%) as a white solid. ¹H NMR (400 MHz, DMSO-d₆) δ 8.67 (t, J=5.6 Hz, 1H), 8.27 (d, J=1.2 Hz, 1H), 7.99 (d, J=1.2 Hz, 1H), 7.71 (d, J=8.8 Hz, 2H), 7.24 (t, J=7.6 Hz, 1H), 7.16 (t, J=8.4 Hz, 2H), 7.04 (d, J=8.8 Hz, 3H), 6.88-6.92 (m, 2H), 6.66 (t, J=7.2 Hz, 1H), 6.42 (d, J=8.0 Hz, 2H), 5.06 (s, 2H), 3.88 (t, J=7.6 Hz, 2H), 3.74 (t, J=4.8 Hz, 4H), 3.54-3.60 (m, 4H), 3.11 (t, J=4.8 Hz, 4H), 2.91-2.95 (m, 1H). LC-MS m/z: 546.3 [M+H]⁺. HPLC Purity (254 nm): 97.71%; t_{R}=7.744 min.

AC2020601-0384a

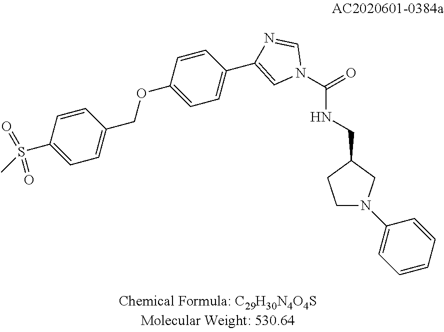 [SMILES: CS(=O)(=O)c1ccc(COc2ccc(-c3cn(C(=O)NC[C@@H]4CCN(c5ccccc5)C4)cn3)cc2)cc1]

### The Synthesis of (S)-tert-butyl ((1-phenylpyrrolidin-3-yl)methyl)carbamate (384a-2)

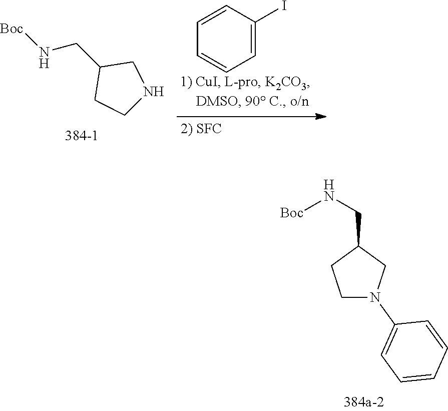 [SMILES: CC(C)(C)OC(=O)NCC1CCNC1 ; Ic1ccccc1 ; CC(C)(C)OC(=O)NC[C@@H]1CCN(c2ccccc2)C1]

A mixture of 384-1 (2.15 g, 10.74 mmol), iodobenzene (4.38 g, 21.48 mmol), CuI (204 mg, 1.07 mmol), L-pro (124 mg, 1.07 mmol) and K₂CO₃ (2.97 g, 21.48 mmol) in DMSO (10 mL) was stirred at 90° C. overnight under N₂. The reaction mixture was quenched with water and extracted with EtOAc (50 mL), the combined organic layer was washed with brine, dried over anhydrous Na₂SO₄ and evaporated under reduced pressure. The crude was purified by silica gel chromatography (25% EA/PE) to afford the racemate 384a-2 (2.4 g, yield 77%).

250 mg racemate was subjected to SFC separation to afford enantiomer 384a-2 (120 mg) as yellow oil.

### The Synthesis of (S)-(1-phenylpyrrolidin-3-yl)methanamine (384a-3)

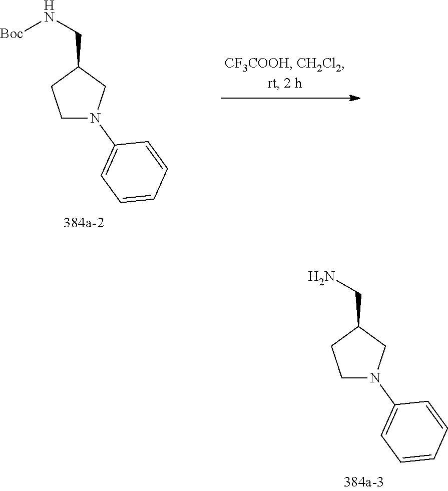 [SMILES: CC(C)(C)OC(=O)NC[C@@H]1CCN(c2ccccc2)C1 ; NC[C@@H]1CCN(c2ccccc2)C1]

To a stirred solution of 384a-2 (120 mg, 0.43 mmol) in CH₂Cl₂ (3 mL) was added TFA (3 mL). The reaction mixture was stirred at room temperature for 2 hours until the reaction was complete (by LCMS). The mixture was concentrated under reduced pressure to afford 384a-3 (75 mg, yield: 98%) as yellow oil, which was used to the next step without purification.

### The Synthesis of (S)-4-(4-((4-(methylsulfonyl)benzyl)oxy)phenyl)-N-((1-phenylpyrrolidin-3-yl)methyl)-1H-imidazole-1-carboxamide (AC2020601-0384a)

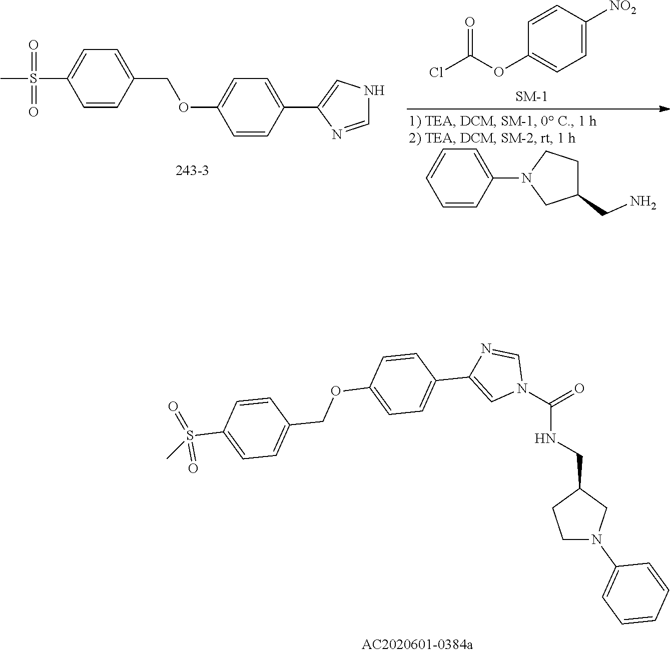 [SMILES: CS(=O)(=O)c1ccc(COc2ccc(-c3c[nH]cn3)cc2)cc1 ; O=C(Cl)Oc1ccc([N+](=O)[O-])cc1 ; NC[C@@H]1CCN(c2ccccc2)C1 ; CS(=O)(=O)c1ccc(COc2ccc(-c3cn(C(=O)NC[C@@H]4CCN(c5ccccc5)C4)cn3)cc2)cc1]

To a solution of 243-3 (153 mg, 0.47 mmol) in DCM (5 mL) was added 4-nitrophenyl carbonochloridate (94 mg, 0.47 mmol) and Et₃N (5 mL) at 0° C. and stirred for 1 hour. Then the reaction mixture was allowed to warm to room temperature and (S)-(1-phenylpyrrolidin-3-yl)methanamine (75 mg, 0.43 mmol) was added. After 1 hour stirring, the reaction mixture was quenched with water, extracted with DCM (20 mL), the combined organic layer was washed with brine, dried over anhydrous Na₂SO₄ and evaporated under reduced pressure. The crude was purified by silica gel chromatography (5% MeOH/DCM) to afford AC2020601-0384a (96 mg, yield: 43%) as a white solid. ¹H NMR (400 MHz, DMSO-d₆) δ 8.65 (t, J=5.2 Hz, 1H), 8.30 (s, 1H), 8.04 (s, 1H), 7.96 (d, J=8.0 Hz, 2H), 7.72-7.76 (m, 4H), 7.13-7.17 (m, 2H), 7.07 (d, J=8.4 Hz, 2H), 6.57-6.60 (m, 1H), 6.52-6.54 (m, 2H), 5.28 (s, 2H), 3.31-3.40 (m, 4H), 3.22-3.27 (m, 4H), 3.05-3.09 (m, 1H), 2.59-2.66 (m, 1H), 2.09-2.15 (m, 1H), 1.78-1.85 (m, 1H). LC-MS m/z: 553.2 [M+Na]⁺. HPLC Purity (254 nm): 100%; t_{R}=9.484 min.

AC2020601-0384b

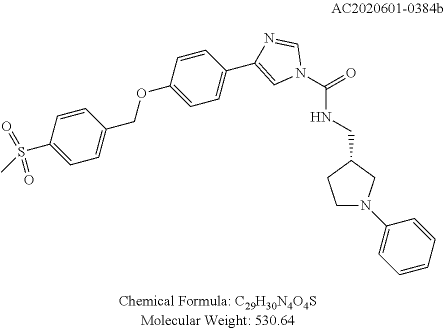 [SMILES: CS(=O)(=O)c1ccc(COc2ccc(-c3cn(C(=O)NC[C@H]4CCN(c5ccccc5)C4)cn3)cc2)cc1]

### The Synthesis of (R)-tert-butyl ((1-phenylpyrrolidin-3-yl)methyl)carbamate (384b-2)

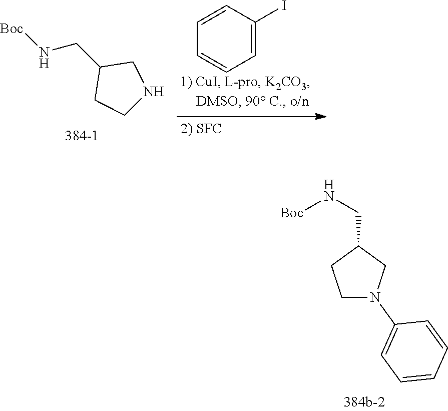 [SMILES: CC(C)(C)OC(=O)NCC1CCNC1 ; Ic1ccccc1 ; CC(C)(C)OC(=O)NC[C@H]1CCN(c2ccccc2)C1]

A mixture of 384-1 (2.15 g, 10.74 mmol), iodobenzene (4.38 g, 21.48 mmol), CuI (204 mg, 1.07 mmol), L-pro (124 mg, 1.07 mmol) and K₂CO₃ (2.97 g, 21.48 mmol) in DMSO (10 mL) was stirred at 90° C. overnight under N₂. The reaction mixture was quenched with water and extracted with EtOAc (50 mL), the combined organic layer was washed with brine, dried over anhydrous Na₂SO₄ and evaporated under reduced pressure. The crude was purified by silica gel chromatography (25% EA/PE) to afford the racemate 384b-2 (2.4 g, yield 77%).

250 mg racemate was subjected to SFC separation to afford enantiomer 384b-2 (120 mg) as yellow oil.

### The Synthesis of (R)-(1-phenylpyrrolidin-3-yl)methanamine (384b-3)

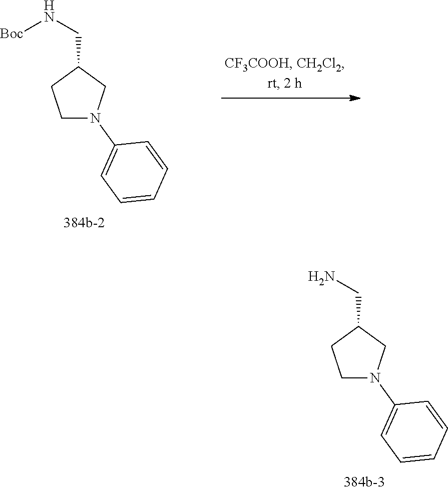 [SMILES: CC(C)(C)OC(=O)NC[C@H]1CCN(c2ccccc2)C1 ; NC[C@H]1CCN(c2ccccc2)C1]

To a stirred solution of 384b-2 (120 mg, 0.43 mmol) in CH₂Cl₂ (3 mL) was added TFA (3 mL). The reaction mixture was stirred at room temperature for 2 hours until the reaction was complete (by LCMS). The mixture was concentrated under reduced pressure to afford 384b-3 (75 mg, yield: 98%) as yellow oil, which was used to the next step without purification.

### The Synthesis of (R)-4-(4-((4-(methylsulfonyl)benzyl)oxy)phenyl)-N-((1-phenylpyrrolidin-3-yl)methyl)-1H-imidazole-1-carboxamide (AC2020601-0384b)

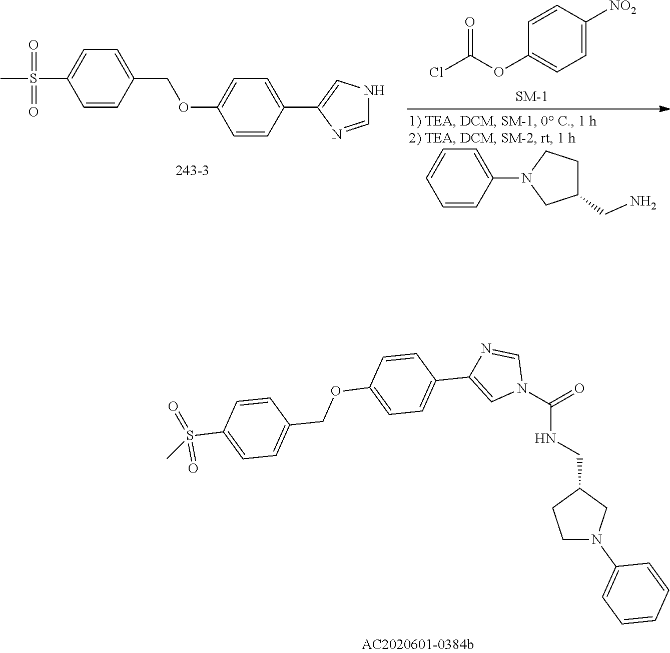 [SMILES: CS(=O)(=O)c1ccc(COc2ccc(-c3c[nH]cn3)cc2)cc1 ; O=C(Cl)Oc1ccc([N+](=O)[O-])cc1 ; NC[C@H]1CCN(c2ccccc2)C1 ; CS(=O)(=O)c1ccc(COc2ccc(-c3cn(C(=O)NC[C@H]4CCN(c5ccccc5)C4)cn3)cc2)cc1]

To a solution of 243-3 (153 mg, 0.47 mmol) in DCM (5 mL) was added 4-nitrophenyl carbonochloridate (94 mg, 0.47 mmol) and Et₃N (5 mL) at 0° C. and stirred for 1 hour. Then the reaction mixture was allowed to warm to room temperature and (R)-(1-phenylpyrrolidin-3-yl)methanamine (75 mg, 0.43 mmol) was added. After 1 hour stirring, the reaction mixture was quenched with water, extracted with DCM (20 mL), the combined organic layer was washed with brine, dried over anhydrous Na₂SO₄ and evaporated under reduced pressure. The crude was purified by silica gel chromatography (5% MeOH/DCM) to afford AC2020601-0384b (108 mg, yield: 48%) as a white solid. ¹H NMR (400 MHz, DMSO-d₆) δ 8.65 (t, J=5.2 Hz, 1H), 8.30 (s, 1H), 8.04 (s, 1H), 7.96 (d, J=8.4 Hz, 2H), 7.72-7.76 (m, 4H), 7.13-7.17 (m, 2H), 7.07 (d, J=8.4 Hz, 2H), 6.57-6.60 (m, 1H), 6.52-6.54 (m, 2H), 5.28 (s, 2H), 3.31-3.40 (m, 4H), 3.21-3.26 (m, 4H), 3.05-3.09 (m, 1H), 2.59-2.66 (m, 1H), 2.09-2.16 (m, 1H), 1.76-1.85 (m, 1H). LC-MS m/z: 553.2 [M+Na]⁺. HPLC Purity (254 nm): 100%; t_{R}=9.480 min.

AC2020601-0401

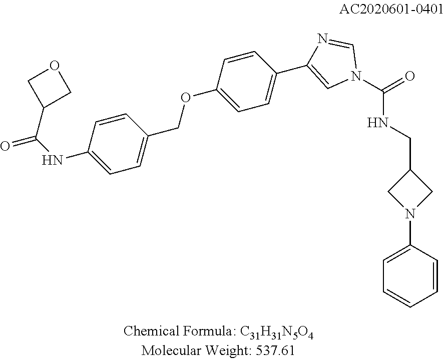 [SMILES: O=C(Nc1ccc(COc2ccc(-c3cn(C(=O)NCC4CN(c5ccccc5)C4)cn3)cc2)cc1)C1COC1]

### The Synthesis of tert-butyl 4-(4-((4-(oxetane-3-carboxamido)benzyl)oxy)phenyl)-1H-imidazole-1-carboxylate (401-4)

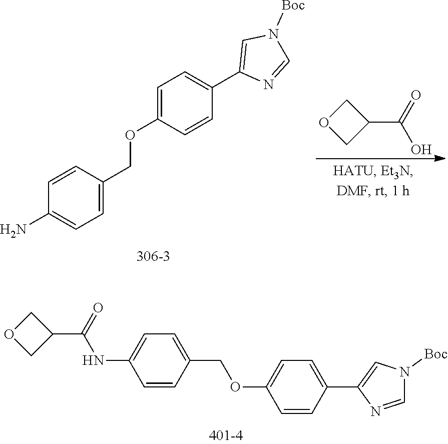 [SMILES: CC(C)(C)OC(=O)n1cnc(-c2ccc(OCc3ccc(N)cc3)cc2)c1 ; O=C(O)C1COC1 ; CC(C)(C)OC(=O)n1cnc(-c2ccc(OCc3ccc(NC(=O)C4COC4)cc3)cc2)c1]

To a suspension of oxetane-3-carboxylic acid (84 mg, 0.82 mmol) in DMF (10 mL) was added Et₃N (111 mg, 1.09 mmol) and HATU (312 mg, 0.82 mmol). The reaction mixture was stirred at room temperature for 0.5 hours, and then 306-3 (200 mg, 0.55 mmol) was added. The reaction mixture was stirred at room temperature for 1 hour until the reaction was complete (by LCMS). Water was added and the reaction mixture was extracted with EtOAc (50 mL), the combined organic layer was washed with brine, dried over anhydrous Na₂SO₄ and evaporated under reduced pressure. The crude was purified by silica gel chromatography (5% MeOH/DCM) to afford 401-4 (160 mg, yield: 65%) as a white solid.

### The Synthesis of N-(4-((4-(1H-imidazol-4-yl)phenoxy)methyl)phenyl)oxetane-3-carboxamide (401-5)

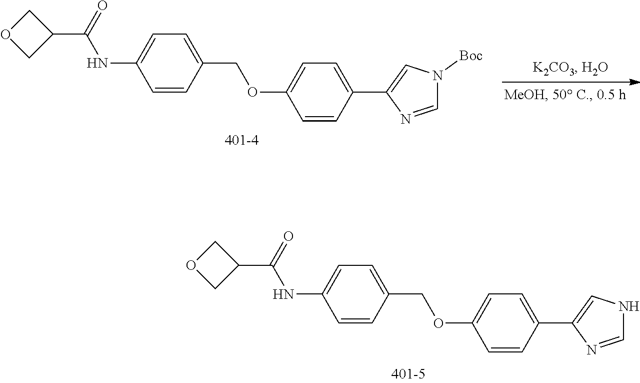 [SMILES: CC(C)(C)OC(=O)n1cnc(-c2ccc(OCc3ccc(NC(=O)C4COC4)cc3)cc2)c1 ; O=C(Nc1ccc(COc2ccc(-c3c[nH]cn3)cc2)cc1)C1COC1]

To a stirred solution of 401-4 (207 mg, 0.46 mmol) in MeOH (10 mL) and H₂O (1 mL) was added K₂CO₃ (128 mg, 0.92 mmol). The reaction mixture was stirred at 50° C. for 0.5 hour until the reaction was complete (by LCMS). The reaction mixture was quenched with water, extracted with EA (20 mL), the combined organic layer was washed with brine, dried over anhydrous Na₂SO₄. The mixture was concentrated under reduced pressure to afford 401-5 (115 mg, crude) as a white solid, which was used to the next step without purification.

### The Synthesis of 4-(4-((4-(oxetane-3-carboxamido)benzyl)oxy)phenyl)-N-((1-phenylazetidin-3-yl)methyl)-1H-imidazole-1-carboxamide (AC2020601-0401)

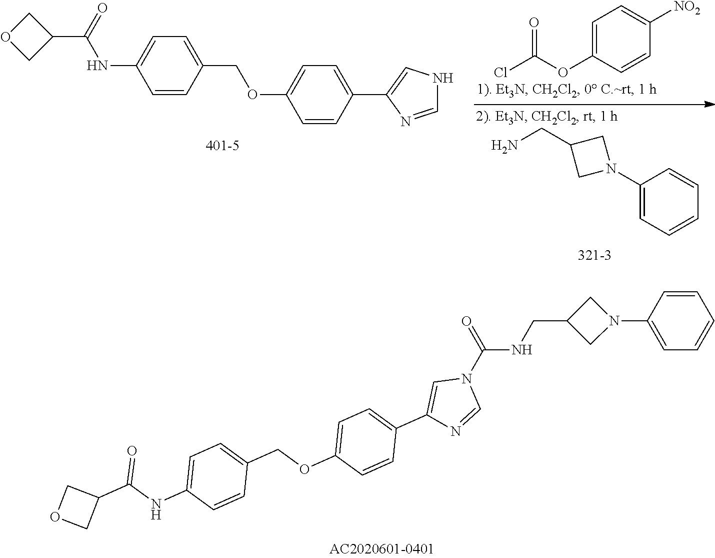 [SMILES: O=C(Nc1ccc(COc2ccc(-c3c[nH]cn3)cc2)cc1)C1COC1 ; O=C(Cl)Oc1ccc([N+](=O)[O-])cc1 ; NCC1CN(c2ccccc2)C1 ; O=C(Nc1ccc(COc2ccc(-c3cn(C(=O)NCC4CN(c5ccccc5)C4)cn3)cc2)cc1)C1COC1]

To a solution of 401-5 (115 mg, 0.33 mmol) in DCM (5 mL) was added 4-nitrophenyl carbonochloridate (80 mg, 0.40 mmol) and Et₃N (5 mL) at 0° C. and stirred for 1 hour. Then the reaction mixture was allowed to warm to room temperature and 321-3 (65 mg, 0.40 mmol) was added. After 1 hour stirring, the reaction mixture was quenched with water, extracted with DCM (20 mL), the combined organic layer was washed with brine, dried over anhydrous Na₂SO₄ and evaporated under reduced pressure. The crude was purified by silica gel chromatography (5% MeOH/DCM) to afford AC2020601-0401 (6 mg, yield: 3.4%) as a white solid. ¹H NMR (400 MHz, DMSO-d₆) δ 9.99 (s, 1H), 8.67 (s, 1H), 8.27 (s, 1H), 8.00 (s, 1H), 7.72 (d, J=8.4 Hz, 2H), 7.63 (d, J=8.4 Hz, 2H), 7.40 (d, J=8.8 Hz, 2H), 7.13-7.18 (m, 2H), 7.03 (d, J=8.8 Hz, 2H), 6.62-6.68 (m, 1H), 6.38-6.43 (m, 2H), 5.06 (s, 2H), 4.68-4.72 (m, 4H), 3.94-3.97 (m, 1H), 3.86-3.90 (m, 2H), 3.55-3.60 (m, 4H), 2.90-2.95 (m, 4H). LC-MS m/z: 538.2 [M+H]⁺. HPLC Purity (254 nm): 100%; t_{R}=9.180 min.

AC2020601-0402

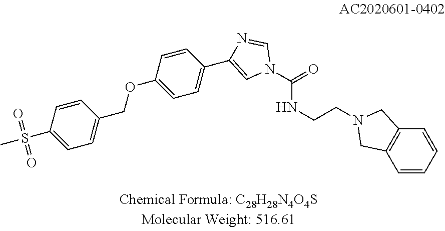 [SMILES: CS(=O)(=O)c1ccc(COc2ccc(-c3cn(C(=O)NCCN4Cc5ccccc5C4)cn3)cc2)cc1]

### The Synthesis of tert-butyl 2-(isoindolin-2-yl)ethylcarbamate (402-2)

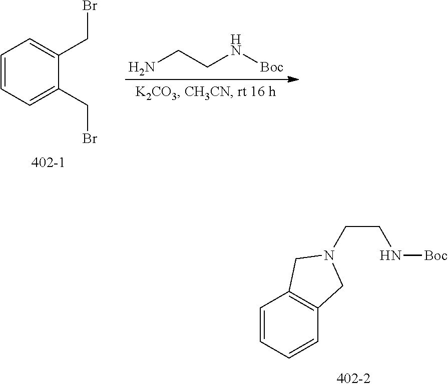 [SMILES: BrCc1ccccc1CBr ; CC(C)(C)OC(=O)NCCN ; CC(C)(C)OC(=O)NCCN1Cc2ccccc2C1]

To a suspension of 402-1 (524 mg, 2.0 mmol) in CH₃CN (10 mL) was added tert-butyl 2-aminoethylcarbamate (640 mg, 4.0 mmol) and K₂CO₃ (552 mg, 4.0 mmol). The reaction mixture was stirred at room temperature for 16 hour until the reaction was complete (by LCMS). Water was added and the reaction mixture was extracted with EtOAc (50 mL), the combined organic layer was washed with brine, dried over anhydrous Na₂SO₄ and evaporated under reduced pressure. The crude was purified by silica gel chromatography (30% EA/PE) to afford 402-2 (300 mg, yield: 57.1%) as yellow oil.

### The Synthesis of (1-(2,3-dihydrobenzofuran-5-yl)pyrrolidin-3-yl)methanamine (402-3)

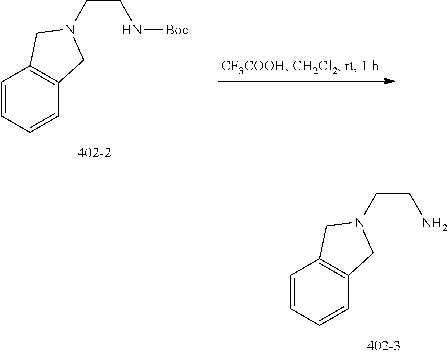 [SMILES: CC(C)(C)OC(=O)NCCN1Cc2ccccc2C1 ; NCCN1Cc2ccccc2C1]

To a stirred solution of 402-2 (300 mg, 1.15 mmol) in CH₂Cl₂ (10 mL) was added TFA (10 mL). The reaction mixture was stirred at room temperature for 1 hour until the reaction was complete (by LCMS). The reaction mixture was quenched with water, extracted with DCM (20 mL), the combined organic layer was washed with brine, dried over anhydrous Na₂SO₄. The mixture was concentrated under reduced pressure to afford 402-3 (150 mg, yield: 81.2%) as yellow oil, which was used to the next step without purification.

### The Synthesis of N-(2-(isoindolin-2-yl)ethyl)-4-(4-(4-(methylsulfonyl)benzyloxy)phenyl)-1H-imidazole-1-carboxamide (AC2020601-0402)

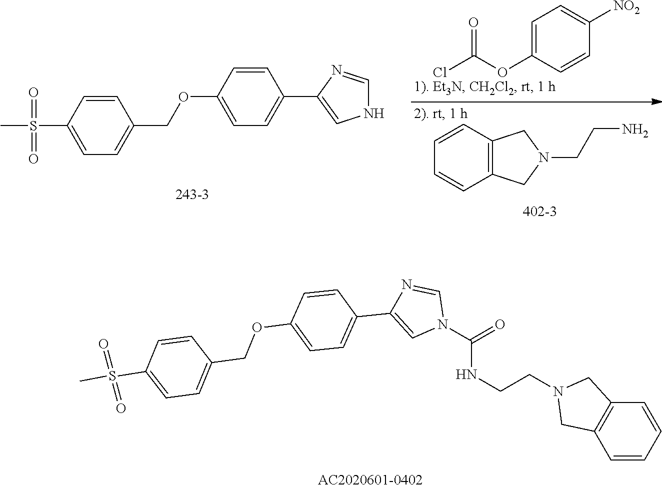 [SMILES: CS(=O)(=O)c1ccc(COc2ccc(-c3c[nH]cn3)cc2)cc1 ; NCCN1Cc2ccccc2C1 ; O=C(Cl)Oc1ccc([N+](=O)[O-])cc1 ; CS(=O)(=O)c1ccc(COc2ccc(-c3cn(C(=O)NCCN4Cc5ccccc5C4)cn3)cc2)cc1]

To a solution of 294-3 (150 mg, 0.38 mmol) in DCM (5 mL) was added 4-nitrophenyl carbonochloridate (114 mg, 0.57 mmol) and Et₃N (5 mL) at room temperature and stirred for 1 hour. Then 402-3 (150 mg, 0.76 mmol) was added. After 1 hour stirring, the reaction mixture was quenched with water, extracted with DCM (20 mL), the combined organic layer was washed with brine, dried over anhydrous Na₂SO₄ and evaporated under reduced pressure. The crude was purified by silica gel chromatography (5% MeOH/DCM) to afford AC2020601-0402 (14.0 mg, yield: 1.8%) as a white solid. ¹H NMR (400 MHz, DMSO-d₆) δ 8.55 (t, J=5.2 Hz, 1H), 8.28 (d, J=1.2 Hz, 1H), 8.02 (d, J=0.8 Hz, 1H), 7.95 (d, J=8.4 Hz, 2H), 7.71-7.74 (m, 4H), 7.22-7.25 (m, 2H), 7.17-7.20 (m, 2H), 7.06 (d, J=8.8 Hz, 2H), 5.27 (s, 2H), 3.93 (s, 4H), 3.45-3.49 (m, 2H), 3.22 (s, 3H), 2.90 (t, J=6.4 Hz, 2H). LC-MS m/z: 517.2 [M+H]⁺. HPLC Purity (254 nm): 74.26%; t_{R}=1.590 min.

AC2020601-0407

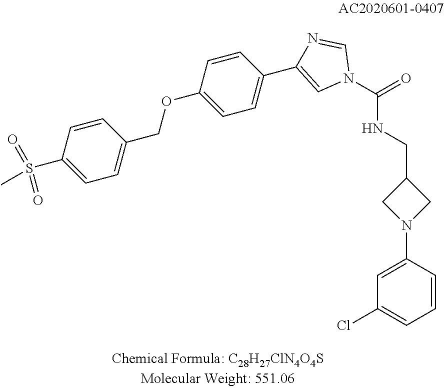 [SMILES: CS(=O)(=O)c1ccc(COc2ccc(-c3cn(C(=O)NCC4CN(c5cccc(Cl)c5)C4)cn3)cc2)cc1]

### The Synthesis of tert-butyl ((1-(3-chlorophenyl)azetidin-3-yl)methyl)carbamate (407-2)

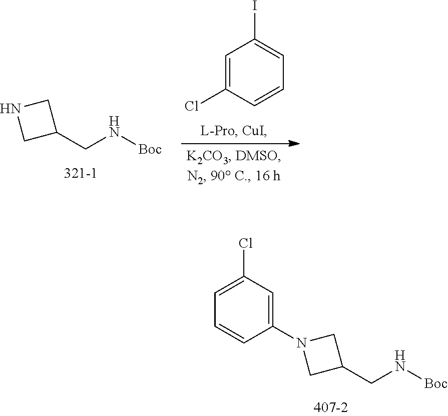 [SMILES: CC(C)(C)OC(=O)NCC1CNC1 ; Clc1cccc(I)c1 ; CC(C)(C)OC(=O)NCC1CN(c2cccc(Cl)c2)C1]

A mixture of 321-1 (2.00 g, 10.74 mmol), 1-chloro-3-iodobenzene (5.12 g, 21.48 mmol), CuI (204 mg, 1.07 mmol), L-pro (124 mg, 1.07 mmol) and K₂CO₃ (2.97 g, 21.48 mmol) in DMSO (10 mL) was stirred at 90° C. overnight under N₂. The reaction mixture was quenched with water and extracted with EtOAc (50 mL), the combined organic layer was washed with brine, dried over anhydrous Na₂SO₄ and evaporated under reduced pressure. The crude was purified by silica gel chromatography (25% EA/PE) to afford 407-2 (2.10 g, yield: 66%) as yellow oil.

### The Synthesis of (1-phenylazetidin-3-yl)methanamine (407-3)

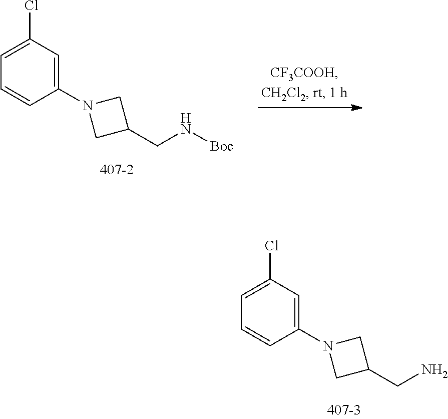 [SMILES: CC(C)(C)OC(=O)NCC1CN(c2cccc(Cl)c2)C1 ; NCC1CN(c2cccc(Cl)c2)C1]

To a stirred solution of 407-2 (2.00 g, 6.74 mmol) in CH₂Cl₂ (10 mL) was added TFA (5 mL). The reaction mixture was stirred at room temperature for 2 hours until the reaction was complete (by LCMS). The mixture was concentrated under reduced pressure to afford 407-3 (1.00 g, crude) as yellow oil, which was used to the next step without purification.

### The Synthesis of N-((1-(3-chlorophenyl)azetidin-3-yl)methyl)-4-(4-((4-(methylsulfonyl)benzyl)oxy)phenyl)-1H-imidazole-1-carboxamide (AC2020601-0407)

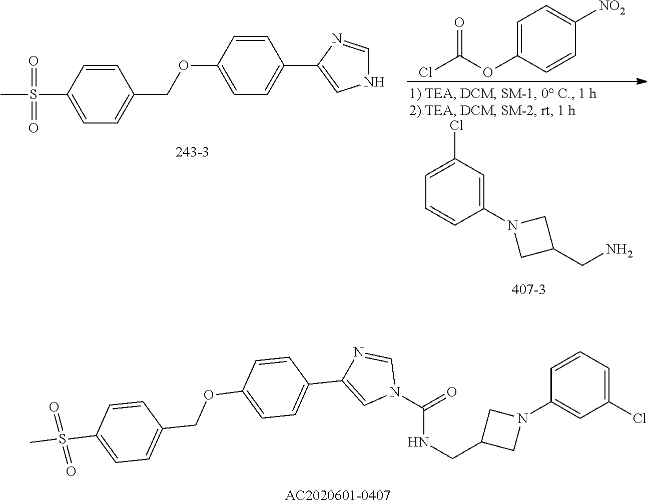 [SMILES: CS(=O)(=O)c1ccc(COc2ccc(-c3c[nH]cn3)cc2)cc1 ; NCC1CN(c2cccc(Cl)c2)C1 ; O=C(Cl)Oc1ccc([N+](=O)[O-])cc1 ; CS(=O)(=O)c1ccc(COc2ccc(-c3cn(C(=O)NCC4CN(c5cccc(Cl)c5)C4)cn3)cc2)cc1]

To a solution of 243-3 (150 mg, 0.46 mmol) in DCM (5 mL) was added 4-nitrophenyl carbonochloridate (101 mg, 0.50 mmol) and Et₃N (2 mL) at 0° C. and stirred for 1 hour. Then the reaction mixture was allowed to warm to room temperature and 407-3 (90 mg, 0.46 mmol) was added. After 1 hour stirring, the reaction mixture was quenched with water, extracted with DCM (20 mL), the combined organic layer was washed with brine, dried over anhydrous Na₂SO₄ and evaporated under reduced pressure. The crude was purified by silica gel chromatography (5% MeOH/DCM) to afford AC2020601-0407 (84 mg, yield: 33%) as a white solid. ¹H NMR (400 MHz, DMSO-d₆) δ 8.67 (t, J=5.6 Hz, 1H), 8.27 (d, J=1.2 Hz, 1H), 8.00 (d, J=0.8 Hz, 1H), 7.96 (d, J=8.4 Hz, 2H), 7.72-7.75 (m, 4H), 7.14-7.18 (m, 1H), 7.07 (d, J=8.0 Hz, 2H), 6.66 (dd, J=7.6 Hz, 1.2 Hz, 1H), 6.42-6.43 (m, 1H), 6.36 (dd, J=8.0 Hz, 1.6 Hz, 1H), 5.28 (s, 2H), 3.90-3.94 (m, 2H), 3.60-3.63 (m, 2H), 3.54-3.57 (m, 2H), 3.22 (s, 3H), 2.93-2.97 (m, 1H). LC-MS m/z: 551.3 [M+H]⁺. HPLC Purity (254 nm): 87.57%; t_{R}=9.552 min.

AC2020601-0428

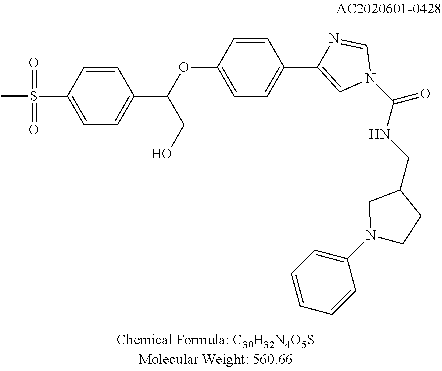 [SMILES: CS(=O)(=O)c1ccc(C(CO)Oc2ccc(-c3cn(C(=O)NCC4CCN(c5ccccc5)C4)cn3)cc2)cc1]

### The Synthesis of 2-hydroxy-1-(4-(methylsulfonyl)phenyl)ethanone (428-2)

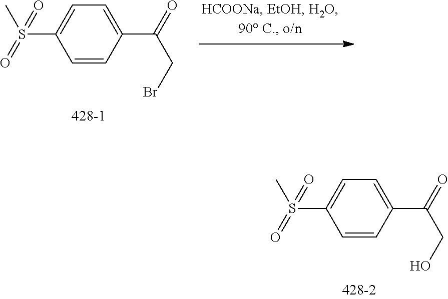

To a suspension of 428-1 (14 g, 50.52 mmol) in EtOH (50 mL) and H₂O (50 mL) was added HCOONa (6.87 g, 101.03 mmol). The reaction mixture was stirred at 90° C. for 16 hours until the reaction was complete (by LCMS). The reaction mixture was extracted with EtOAc (20 mL), the combined organic layer was washed with brine, dried over anhydrous Na₂SO₄ and evaporated under reduced pressure. The crude was purified by silica gel chromatography (1% MeOH/DCM) to afford 428-2 (8 g, yield: 74%) as colorless oil.

### The Synthesis of 2-((tert-butyldimethylsilyl)oxy)-1-(4-(methylsulfonyl)phenyl)ethanone (428-3)

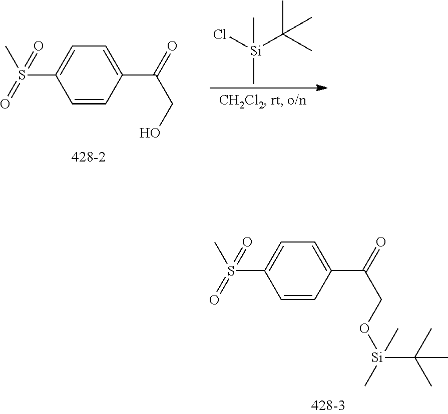

To a solution of 428-2 (8 g, 37.34 mmol) in CH₂Cl₂ (100 mL) was added TEA (7.56 g, 74.68 mmol) and TBSCl (11.26 g, 74.68 mmol). The reaction mixture was stirred at room temperature for 16 hours until the reaction was complete (by LCMS). Water was added and the reaction mixture was extracted with CH₂Cl₂ (100 mL), the combined organic layer was washed with brine, dried over anhydrous Na₂SO₄ and evaporated under reduced pressure. The crude was purified by silica gel chromatography (1% MeOH/DCM) to afford 428-3 (12 g, yield: 98%) as yellow oil.

### The Synthesis of 2-((tert-butyldimethylsilyl)oxy)-1-(4-(methylsulfonyl)phenyl)ethanol (428-4)

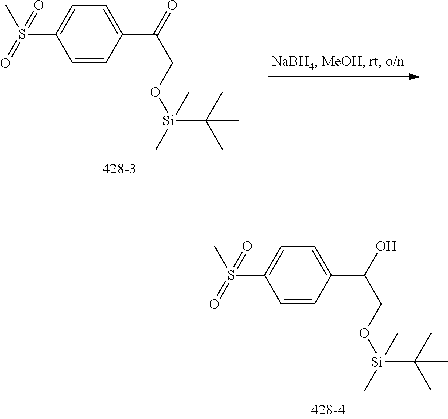 [SMILES: CC(C)(C)[Si](C)(C)OCC(=O)c1ccc(S(C)(=O)=O)cc1 ; CC(C)(C)[Si](C)(C)OCC(O)c1ccc(S(C)(=O)=O)cc1]

To a solution of 428-3 (12 g, 36.53 mmol) in MeOH (100 mL) was added NaBH₄ (2.76 g, 73.06 mmol). The reaction mixture was stirred at room temperature for 16 hours until the reaction was complete (by LCMS). Saturated NH₄Cl solution was added and the reaction mixture was extracted with EA (100 mL), the combined organic layer was washed with brine, dried over anhydrous Na₂SO₄ and evaporated under reduced pressure. The crude was purified by silica gel chromatography (5% MeOH/DCM) to afford 428-4 (6 g, yield: 50%) as yellow oil.

### The Synthesis of tert -butyl 4-(4-(2-((tert-butyldimethylsilyl)oxy)-1-(4-(methylsulfonyl)phenyl)ethoxy)phenyl)-1H-imidazole-1-carboxylate (428-5)

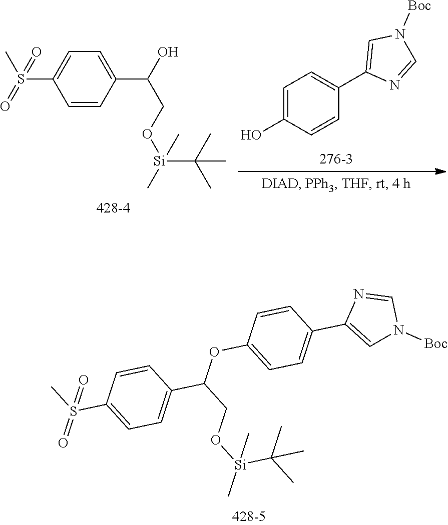

To a suspension of 276-3 (780 mg, 3.0 mmol) in THF (20 mL) was added 428-4 (992 mg, 3.0 mmol), PPh₃ (942.0 mg, 3.6 mmol) and DIAD (789.0 mg, 3.9 mmol). The reaction mixture was stirred at room temperature for 4 hours until the reaction was complete (by LCMS). Saturated NaCl solution was added and the reaction mixture was extracted with EtOAc (50 mL), the combined organic layer was washed with brine, dried over anhydrous Na₂SO₄ and evaporated under reduced pressure. The crude was purified by silica gel chromatography (10% MeOH/DCM) to afford 428-5 (800 mg, yield: 47%) as yellow oil.

### The Synthesis of 4-(4-(2-((tert-butyldimethylsilyl)oxy)-1-(4-(methylsulfonyl)phenyl)ethoxy)phenyl)-1H-imidazole (428-6)

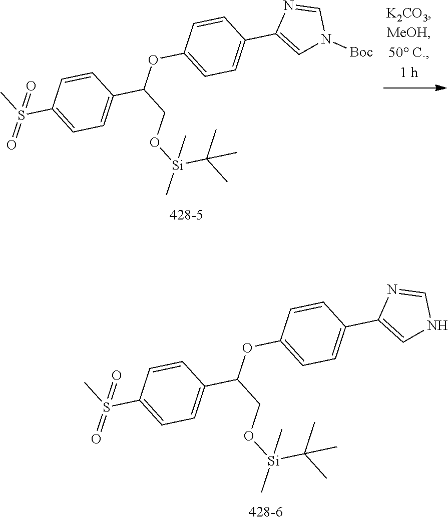 [SMILES: CC(C)(C)OC(=O)n1cnc(-c2ccc(OC(CO[Si](C)(C)C(C)(C)C)c3ccc(S(C)(=O)=O)cc3)cc2)c1 ; CC(C)(C)[Si](C)(C)OCC(Oc1ccc(-c2c[nH]cn2)cc1)c1ccc(S(C)(=O)=O)cc1]

To a solution of 428-5 (0.8 g, 1.40 mmol) in MeOH (10 mL) was added K₂CO₃ (386 mg, 2.79 mmol). The reaction mixture was stirred at 50° C. for 1 hour until the reaction was complete (by LCMS). Water was added and the reaction mixture was extracted with EA (30 mL), the combined organic layer was washed with brine, dried over anhydrous Na₂SO₄ and evaporated under reduced pressure to afford 428-6 (0.3 g, yield: 45%) as yellow oil.

### The Synthesis of 4-(4-(2-((tert-butyldimethylsilyl)oxy)-1-(4-(methylsulfonyl)phenyl)ethoxy)phenyl)-N-((1-phenylpyrrolidin-3-yl)methyl)-1H-imidazole-1-carboxamide (428-7)

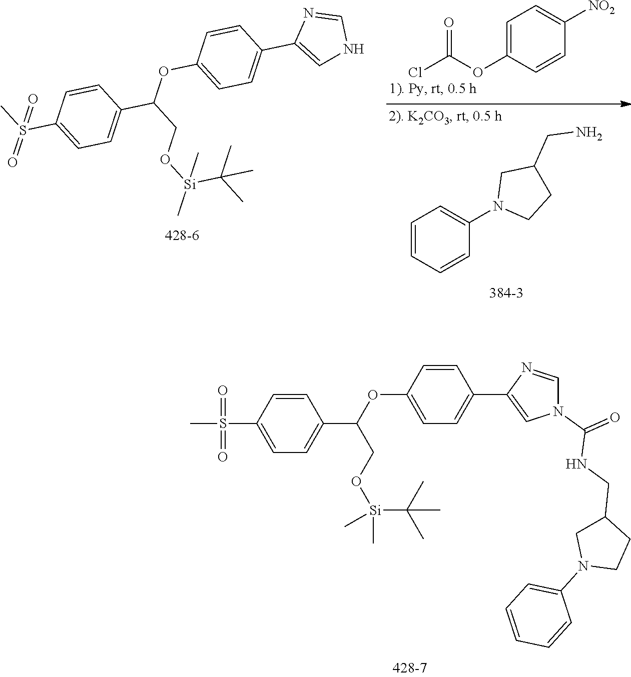 [SMILES: CC(C)(C)[Si](C)(C)OCC(Oc1ccc(-c2c[nH]cn2)cc1)c1ccc(S(C)(=O)=O)cc1 ; NCC1CCN(c2ccccc2)C1 ; O=C(Cl)Oc1ccc([N+](=O)[O-])cc1 ; CC(C)(C)[Si](C)(C)OCC(Oc1ccc(-c2cn(C(=O)NCC3CCN(c4ccccc4)C3)cn2)cc1)c1ccc(S(C)(=O)=O)cc1]

To a solution of 428-6 (156 mg, 0.33 mmol) in Py (5 mL) was added SM1 (82.0 mg, 0.4 mmol) at 0° C. and stirred for 1 hour. Then the reaction mixture was allowed to warm to room temperature and K₂CO₃ (138 mg, 1.0 mmol), 384-3 (71.0 mg, 0.4 mmol) was added. After 1 hour stirring, the reaction mixture was quenched with water, extracted with DCM (20 mL), the combined organic layer was washed with brine, dried over anhydrous Na₂SO₄ and evaporated under reduced pressure. The crude was purified by silica gel chromatography (10% MeOH/DCM) to afford 428-7 (70 mg, yield: 31%) as a white solid.

### The Synthesis of 4-(4-(2-hydroxy-1-(4-(methylsulfonyl)phenyl)ethoxy)phenyl)-N-((1-phenylpyrrolidin-3-yl)methyl)-1H-imidazole-1-carboxamide (AC2020601-0428)

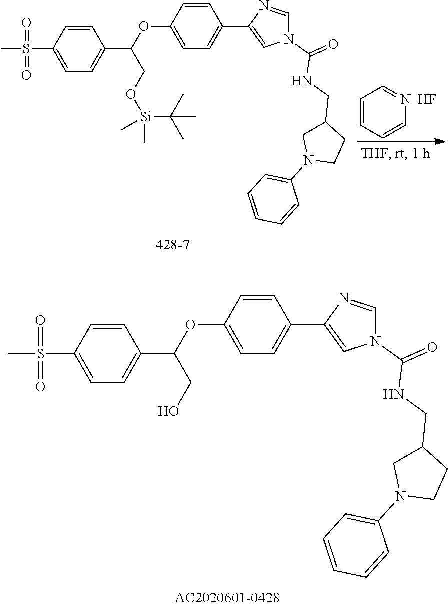

To a solution of 428-7 (70 mg, 0.10 mmol) in THF (10 mL) was added pyridine hydrofluoride (21 mg, 0.21 mmol). The reaction mixture was stirred at room temperature for 1 hour until the reaction was complete (by LCMS). Saturated NH₄Cl solution was added and the reaction mixture was extracted with EA (10 mL), the combined organic layer was washed with brine, dried over anhydrous Na₂SO₄ and evaporated under reduced pressure. The crude was purified by Prep-HPLC to afford AC2020601-0428 (2 mg, yield: 3.4%) as a white solid. ¹H NMR (400 MHz, DMSO-d₆) δ 8.64 (t, J=5.2 Hz, 1H), 8.27 (s, 1H), 7.97 (s, 1H), 7.91 (d, J=8.0 Hz, 2H), 7.64-7.70 (m, 4H), 7.13-7.17 (m, 2H), 6.96 (d, J=8.4 Hz, 2H), 6.51-6.60 (m, 3H), 5.48-5.51 (m, 1H), 5.21-5.24 (m, 1H), 3.75-3.81 (m, 1H), 3.67-3.71 (m, 1H), 3.37-3.39 (m, 1H), 3.20-3.28 (m, 4H), 3.04-3.08 (m, 1H), 2.58-2.65 (m, 1H), 2.09-2.14 (m, 1H), 1.77-1.82 (m, 1H), 1.34-1.36 (m, 2H), 0.84-0.87 (m, 1H). LC-MS m/z: 561.2 [M+H]⁺. HPLC Purity (254 nm):97.93%; t_{R}=10.469 min.

AC2020601-0430

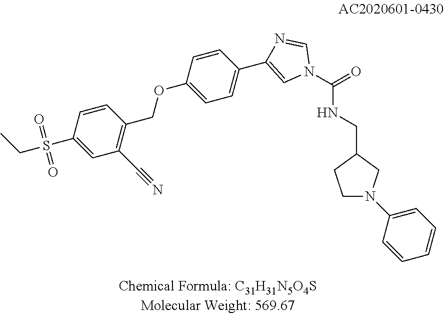 [SMILES: CCS(=O)(=O)c1ccc(COc2ccc(-c3cn(C(=O)NCC4CCN(c5ccccc5)C4)cn3)cc2)c(C#N)c1]

### The Synthesis of methyl 2-bromo-4-(ethylthio)benzoate (430-2)

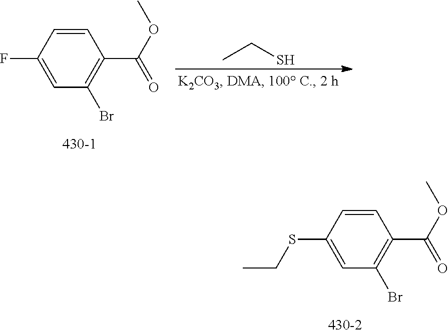

To a suspension of 430-1 (16 g, 68.66 mmol) in DMA (50 mL) was added K₂CO₃ (18.98 g, 137.32 mmol) and ethanethiol (8.53 g, 137.32 mmol). The reaction mixture was stirred at 100° C. for 2 hours until the reaction was complete (by LCMS). Saturated NaCl solution was added and the reaction mixture was extracted with EtOAc (20 mL), the combined organic layer was washed with brine, dried over anhydrous Na₂SO₄ and evaporated under reduced pressure. The crude was purified by silica gel chromatography (10% MeOH/DCM) to afford 430-2 (17 g, yield: 90%) as a yellow solid.

### The Synthesis of methyl 2-bromo-4-(ethylsulfonyl)benzoate (430-3)

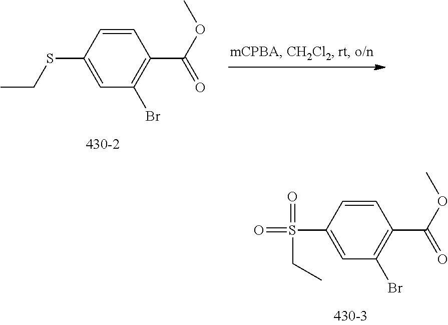 [SMILES: CCSc1ccc(C(=O)OC)c(Br)c1 ; CCS(=O)(=O)c1ccc(C(=O)OC)c(Br)c1]

To a solution of 430-2 (17 g, 61.78 mmol) in CH₂Cl₂ (100 mL) was added mCPBA (21.32 g, 123.56 mmol). The reaction mixture was stirred at room temperature for 16 hours until the reaction was complete (by LCMS). Saturated Na₂SO₃ solution was added and the reaction mixture was extracted with CH₂Cl₂ (20 mL), the combined organic layer was washed with brine, dried over anhydrous Na₂SO₄ and evaporated under reduced pressure. The crude was purified by silica gel chromatography (10% MeOH/DCM) to afford 430-3 (13 g, yield: 68%) as a yellow solid.

### The Synthesis of (2-bromo-4-(ethylsulfonyl)phenyl)methanol (430-4)

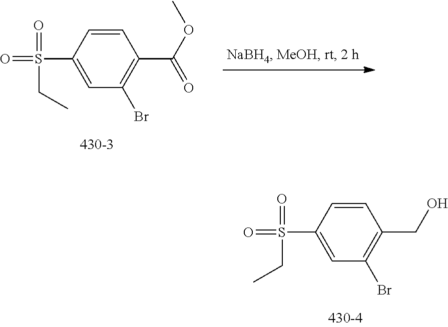 [SMILES: CCS(=O)(=O)c1ccc(C(=O)OC)c(Br)c1 ; CCS(=O)(=O)c1ccc(CO)c(Br)c1]

To a solution of 430-3 (13 g, 42.32 mmol) in MeOH (100 mL) was added NaBH₄ (3.20 g, 84.65 mmol). The reaction mixture was stirred at room temperature for 2 hours until the reaction was complete (by LCMS). Saturated NH₄Cl solution was added and the reaction mixture was extracted with EA (100 mL), the combined organic layer was washed with brine, dried over anhydrous Na₂SO₄ and evaporated under reduced pressure. The crude was purified by silica gel chromatography (10% MeOH/DCM) to afford 430-4 (6 g, yield: 51%) as yellow oil.

### The Synthesis of tert -butyl 4-(4-((2-bromo-4-(ethylsulfonyl)benzyl)oxy)phenyl)-1H-imidazole-1-carboxylate (430-5)

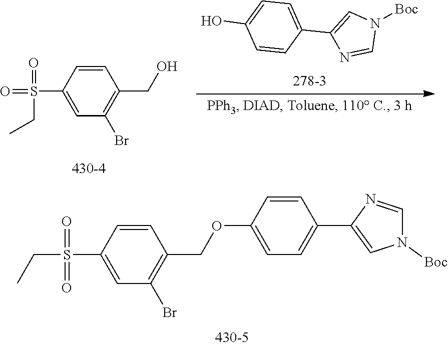 [SMILES: CC(C)(C)OC(=O)n1cnc(-c2ccc(O)cc2)c1 ; CCS(=O)(=O)c1ccc(CO)c(Br)c1 ; CCS(=O)(=O)c1ccc(COc2ccc(-c3cn(C(=O)OC(C)(C)C)cn3)cc2)c(Br)c1]

To a suspension of 278-3 (780 mg, 3.0 mmol) in Tol (20 mL) was added 430-4 (837 mg, 3.0 mmol), PPh₃ (942.0 mg, 3.6 mmol) and DIAD (789.0 mg, 3.9 mmol). The reaction mixture was stirred 110° C. for 3 hours until the reaction was complete (by LCMS). Saturated NaCl solution was added and the reaction mixture was extracted with EtOAc (50 mL), the combined organic layer was washed with brine, dried over anhydrous Na₂SO₄ and evaporated under reduced pressure. The crude was purified by silica gel chromatography (10% MeOH/DCM) to afford 430-5 (525 mg, yield: 33%) as yellow oil.

### The Synthesis of 2-((4-(1H-imidazol-4-yl)phenoxy)methyl)-5-(ethylsulfonyl)benzonitrile (430-6)

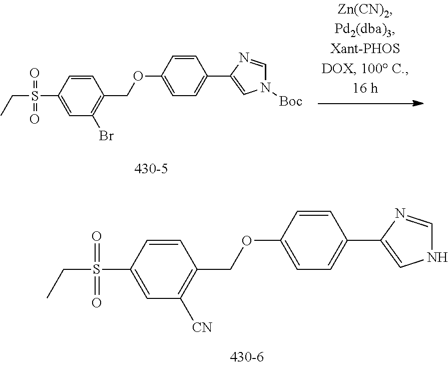 [SMILES: CCS(=O)(=O)c1ccc(COc2ccc(-c3cn(C(=O)OC(C)(C)C)cn3)cc2)c(Br)c1 ; CCS(=O)(=O)c1ccc(COc2ccc(-c3c[nH]cn3)cc2)c(C#N)c1]

A mixture of 430-5 (535 mg, 1.03 mmol), Zn(CN)₂ (602 mg, 5.13 mmol), Pd₂(dba)₃ (47 mg, 0.05 mmol) and Xant-Phos (59 mg, 1.03 mmol) in DOX (10 mL) was stirred at 100° C. overnight under N₂. The reaction mixture was quenched with water and extracted with EtOAc (50 mL), the combined organic layer was washed with brine, dried over anhydrous Na₂SO₄ and evaporated under reduced pressure. The crude was purified by silica gel chromatography (25% EA/PE) to afford 430-6 (230 mg, yield: 61%) as yellow oil.

### The Synthesis of 4-(4-((2-cyano-4-(ethylsulfonyl)benzyl)oxy)phenyl)-N-((1-phenylpyrrolidin-3-yl)methyl)-1H-imidazole-1-carboxamide (AC2020601-0430)

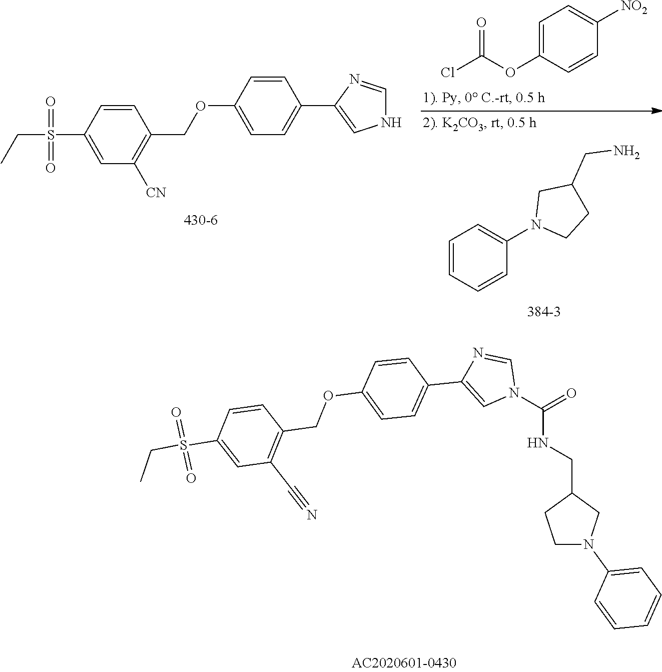 [SMILES: O=C(Cl)Oc1ccc([N+](=O)[O-])cc1 ; CCS(=O)(=O)c1ccc(COc2ccc(-c3c[nH]cn3)cc2)c(C#N)c1 ; NCC1CCN(c2ccccc2)C1 ; CCS(=O)(=O)c1ccc(COc2ccc(-c3cn(C(=O)NCC4CCN(c5ccccc5)C4)cn3)cc2)c(C#N)c1]

To a solution of 430-6 (121 mg, 0.33 mmol) in Py (5 mL) was added SM1 (82.0 mg, 0.4 mmol) at 0° C. and stirred for 0.5 hour. Then the reaction mixture was allowed to warm to room temperature and K₂CO₃ (138 mg, 1.0 mmol), 384-3 (71.0 mg, 0.4 mmol) was added. After 0.5 hour stirring, the reaction mixture was quenched with water, extracted with DCM (20 mL), the combined organic layer was washed with brine, dried over anhydrous Na₂SO₄ and evaporated under reduced pressure. The crude was purified by silica gel chromatography (10% MeOH/DCM) to afford AC2020601-0430 (6 mg, yield: 3.2%) as a white solid. ¹H NMR (400 MHz, DMSO-d₆+D₂O) δ 8.41 (s, 1H), 8.31 (s, 1H), 8.22-8.24 (m, 1H), 8.03-8.06 (m, 2H), 7.78 (d, J=8.4 Hz, 2H), 7.07-7.19 (m, 4H), 6.50-6.62 (m, 3H), 5.40 (s, 2H), 3.31-3.44 (m, 6H), 3.21-3.27 (m, 1H), 3.04-3.08 (m, 1H), 2.62-2.70 (m, 1H), 2.10-2.16 (m, 1H), 1.78-1.85 (m, 1H), 1.09-1.16 (m, 3H). HPLC Purity (254 nm): 96.49%; t_{R}=8.769 min.

AC2020601-0431

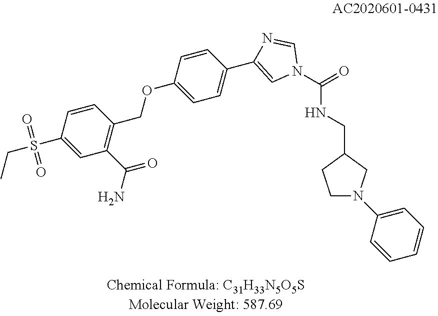 [SMILES: CCS(=O)(=O)c1ccc(COc2ccc(-c3cn(C(=O)NCC4CCN(c5ccccc5)C4)cn3)cc2)c(C(N)=O)c1]

### The Synthesis of 2-((4-(1H-imidazol-4-yl)phenoxy)methyl)-5-(ethylsulfonyl)benzonitrile (431-2)

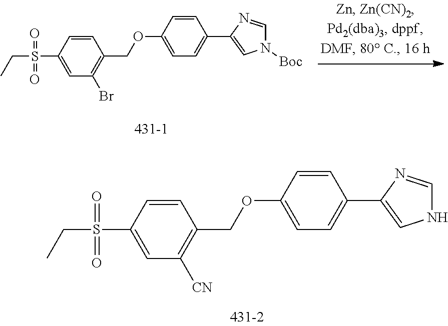 [SMILES: CCS(=O)(=O)c1ccc(COc2ccc(-c3cn(C(=O)OC(C)(C)C)cn3)cc2)c(Br)c1 ; CCS(=O)(=O)c1ccc(COc2ccc(-c3c[nH]cn3)cc2)c(C#N)c1]

To a suspension of 431-1 (521 mg, 1.0 mmol) in DMF (10 mL) was added Zn (130 mg, 2.0 mmol), Zn(CN)₂ (334 mg, 2.0 mmol), Pd₂(dba)₃ (91 mg, 0.1 mmol) and dppf (91 mg, 0.1 mmol). The reaction mixture was allowed to warm to 80° C. for 16 hours until the reaction was complete (by LCMS). Water was added and the reaction mixture was extracted with EtOAc (50 mL), the combined organic layer was washed with brine, dried over anhydrous Na₂SO₄ and evaporated under reduced pressure. The crude was purified by silica gel chromatography (5% MeOH/DCM) to afford 431-2 (300 mg, yield: 82%) as black oil.

### The Synthesis of 2-((4-(1H-imidazol-4-yl)phenoxy)methyl)-5-(ethylsulfonyl)benzamide (431-3)

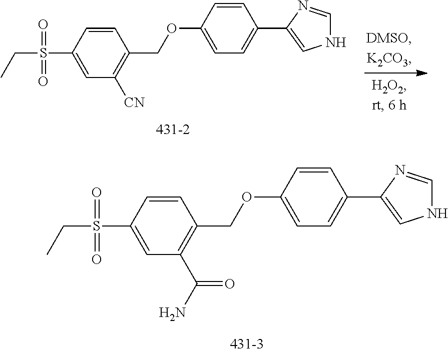 [SMILES: CCS(=O)(=O)c1ccc(COc2ccc(-c3c[nH]cn3)cc2)c(C#N)c1 ; CCS(=O)(=O)c1ccc(COc2ccc(-c3c[nH]cn3)cc2)c(C(N)=O)c1]

To a stirred solution of 431-2 (300 mg, 0.82 mmol) in DMSO (5 mL) and H₂O₂ (5 mL) was added K₂CO₃ (226 mg, 1.64 mmol). The reaction mixture was stirred at room temperature for 6 hours until the reaction was complete (by LCMS). The reaction mixture was quenched with water, extracted with EA (20 mL), the combined organic layer was washed with brine, dried over anhydrous Na₂SO₄ and evaporated under reduced pressure. The crude was purified by silica gel chromatography (5% MeOH/DCM) to afford 431-3 (120 mg, yield: 31%) as black oil.

### The Synthesis of 4-(4-(2-carbamoyl-4-(ethylsulfonyl)benzyloxy)phenyl)-N-((1-phenylpyrrolidin-3-yl)methyl)-1H-imidazole-1-carboxamide (AC2020601-0431)

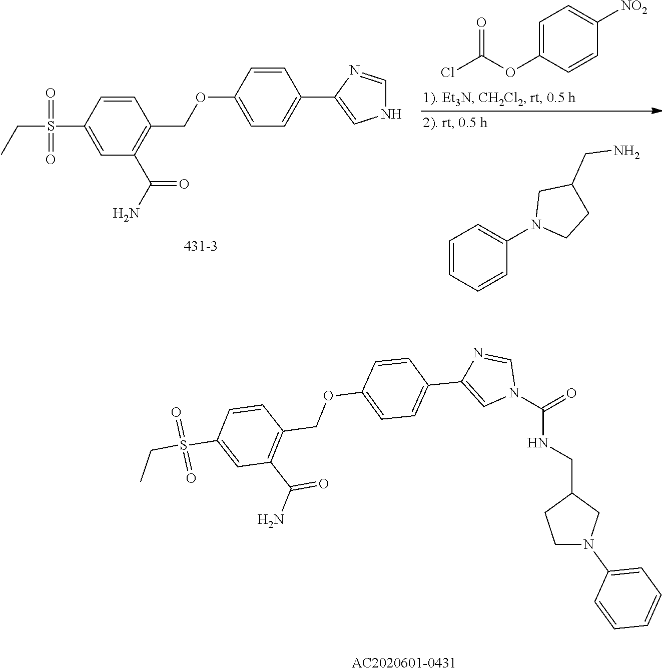 [SMILES: CCS(=O)(=O)c1ccc(COc2ccc(-c3c[nH]cn3)cc2)c(C(N)=O)c1 ; NCC1CCN(c2ccccc2)C1 ; O=C(Cl)Oc1ccc([N+](=O)[O-])cc1 ; CCS(=O)(=O)c1ccc(COc2ccc(-c3cn(C(=O)NCC4CCN(c5ccccc5)C4)cn3)cc2)c(C(N)=O)c1]

To a solution of 431-3 (120 mg, 0.31 mmol) in DCM (5 mL) was added 4-nitrophenyl carbonochloridate (94 mg, 0.47 mmol) and Et₃N (5 mL) at room temperature and stirred for 0.5 hour. Then (1-phenylpyrrolidin-3-yl)methanamine (109 mg, 0.62 mmol) was added. After 0.5 hour stirring, the reaction mixture was quenched with water, extracted with DCM (20 mL), the combined organic layer was washed with brine, dried over anhydrous Na₂SO₄ and evaporated under reduced pressure. The crude was purified by silica gel chromatography (5% MeOH/DCM) to afford AC2020601-0431 (13 mg, yield: 7%) as a white solid. ¹H NMR (400 MHz, DMSO-d₆) δ 8.65 (t, J=5.6 Hz, 1H), 8.30 (d, J=1.2 Hz, 1H), 8.19 (s, 1H), 8.03 (d, J=1.2 Hz, 1H), 7.97-8.00 (m, 2H), 7.86-7.88 (m, 2H), 7.71-7.76 (m, 2H), 7.15 (t, J=7.6 Hz, 2H), 7.03 (d, J=9.2 Hz, 2H), 6.52-6.60 (m, 3H), 5.41 (s, 2H), 3.34-3.40 (m, 5H), 3.21-3.27 (m, 1H), 3.05-3.09 (m, 1H), 2.57-2.70 (m, 2H), 2.09-2.17 (m, 1H), 1.78-1.85 (m, 1H), 1.12 (t, J=7.6 Hz, 3H). LC-MS m/z: 588.2 [M+H]⁺. HPLC Purity (254 nm): 94.54%; t_{R}=8.949 min.

AC2020601-0434

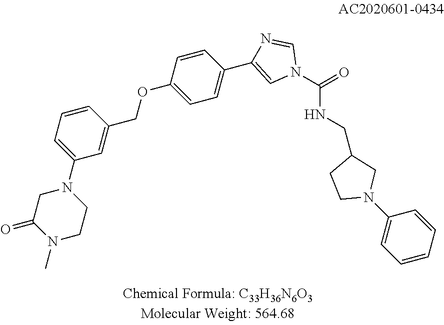 [SMILES: CN1CCN(c2cccc(COc3ccc(-c4cn(C(=O)NCC5CCN(c6ccccc6)C5)cn4)cc3)c2)CC1=O]

### The Synthesis of 4-(3-(hydroxymethyl)phenyl)-1-methylpiperazin-2-one (434-2)

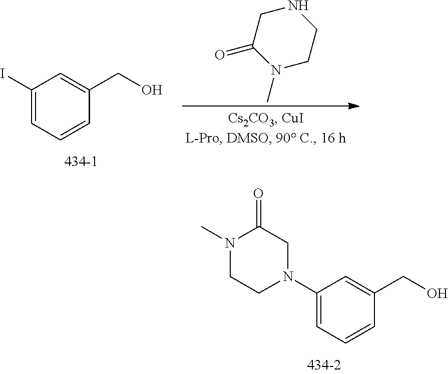

To a suspension of 434-1 (1.0 g, 4.3 mmol) in DMSO (10 mL) was added 1-methylpiperazin-2-one (980 mg, 8.6 mmol), Cs₂CO₃ (2.8 g, 8.6 mmol), CuI (81 mg, 0.43 mmol) and L-Pro (50 mg, 0.43 mmol). The reaction mixture was allowed to warm to 90° C. for 16 hours until the reaction was complete (by LCMS). Water was added and the reaction mixture was extracted with EtOAc (50 mL), the combined organic layer was washed with brine, dried over anhydrous Na₂SO₄ and evaporated under reduced pressure. The crude was purified by silica gel chromatography (30% EA/PE) to afford 434-2 (600 mg, yield: 64.2%) as yellow oil.

### The Synthesis of tert-butyl 4-(4-(3-(4-methyl-3-oxopiperazin-1-yl)benzyloxy)phenyl)-1H-imidazole-1-carboxylate (434-3)

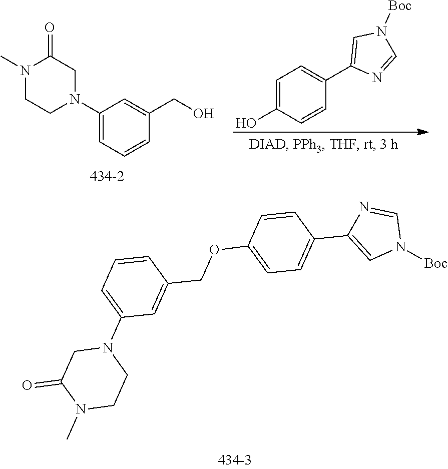 [SMILES: CN1CCN(c2cccc(CO)c2)CC1=O ; CC(C)(C)OC(=O)n1cnc(-c2ccc(O)cc2)c1 ; CN1CCN(c2cccc(COc3ccc(-c4cn(C(=O)OC(C)(C)C)cn4)cc3)c2)CC1=O]

To a suspension of 434-2 (600 mg, 2.7 mmol) in THF (20 mL) was added tert-butyl 4-(4-hydroxyphenyl)-1H-imidazole-1-carboxylate (1.4 g, 5.4 mmol), PPh₃ (838 mg, 3.2 mmol) and DIAD (700 mg, 3.5 mmol). The reaction mixture was stirred at room temperature for 3 hour until the reaction was complete (by LCMS). Water was added and the reaction mixture was extracted with EtOAc (50 mL), the combined organic layer was washed with brine, dried over anhydrous Na₂SO₄ and evaporated under reduced pressure. The crude was purified by silica gel chromatography (30% EA/PE) to afford 434-3 (500 mg, yield: 39.7%) as yellow oil.

### The Synthesis of 4-(3-((4-(1H-imidazol-4-yl)phenoxy)methyl)phenyl)-1-methylpiperazin-2-one (434-4)

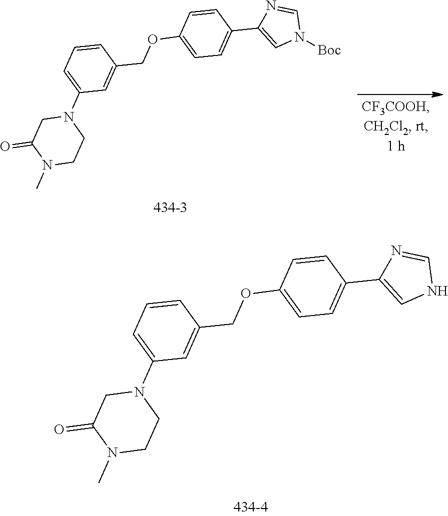 [SMILES: CN1CCN(c2cccc(COc3ccc(-c4cn(C(=O)OC(C)(C)C)cn4)cc3)c2)CC1=O ; CN1CCN(c2cccc(COc3ccc(-c4c[nH]cn4)cc3)c2)CC1=O]

To a stirred solution of 434-3 (500 mg, 1.1 mmol) in CH₂Cl₂ (10 mL) was added TFA (10 mL). The reaction mixture was stirred at room temperature for 1 hour until the reaction was complete (by LCMS). The reaction mixture was quenched with water, extracted with DCM (20 mL), the combined organic layer was washed with brine, dried over anhydrous Na₂SO₄. The mixture was concentrated under reduced pressure to afford 434-4 (300 mg, yield: 75.4%) as yellow oil, which was used to the next step without purification.

### The Synthesis of 4-(4-(3-(4-methyl-3-oxopiperazin-1-yl)benzyloxy)phenyl)-N-((1-phenylpyrrolidin-3-yl)methyl)-1H-imidazole-1-carboxamide (AC2020601-0434)

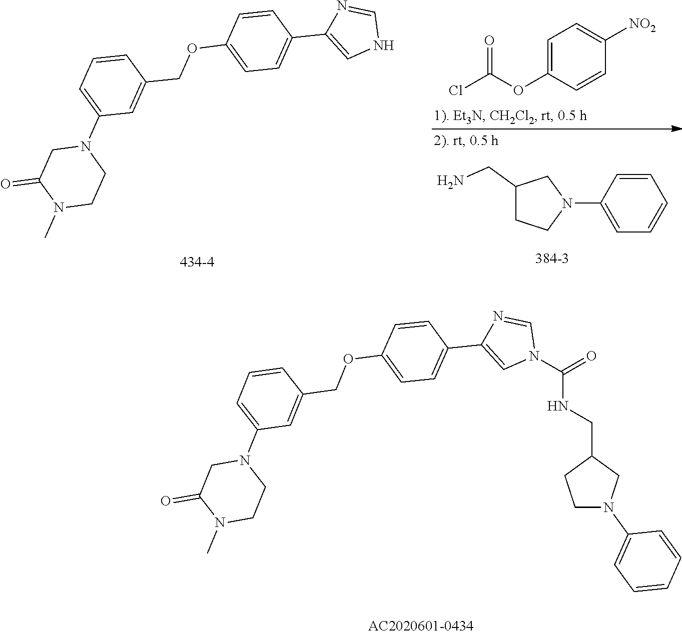 [SMILES: CN1CCN(c2cccc(COc3ccc(-c4c[nH]cn4)cc3)c2)CC1=O ; NCC1CCN(c2ccccc2)C1 ; O=C(Cl)Oc1ccc([N+](=O)[O-])cc1 ; CN1CCN(c2cccc(COc3ccc(-c4cn(C(=O)NCC5CCN(c6ccccc6)C5)cn4)cc3)c2)CC1=O]

To a solution of 434-4 (150 mg, 0.41 mmol) in DCM (5 mL) was added 4-nitrophenyl carbonochloridate (123 mg, 0.61 mmol) and Et₃N (5 mL) at room temperature and stirred for 0.5 hours. Then (1-phenylpyrrolidin-3-yl)methanamine (384-3, 144 mg, 0.82 mmol) was added. After 0.5 hour stirring, the reaction mixture was quenched with water, extracted with DCM (20 mL), the combined organic layer was washed with brine, dried over anhydrous Na₂SO₄ and evaporated under reduced pressure. The crude was purified by silica gel chromatography (5% MeOH/DCM) to afford AC2020601-0434 (20.0 mg, yield: 9%) as a white solid. ¹H NMR (400 MHz, DMSO-d₆) δ 8.65 (t, J=5.6 Hz, 1H), 8.29 (d, J=1.2 Hz, 1H), 8.02 (d, J=1.2 Hz, 1H), 7.72 (d, J=8.8 Hz, 2H), 7.25 (t, J=8.0 Hz, 1H), 7.15-7.17 (m, 2H), 7.04-7.06 (m, 3H), 6.89-6.92 (m, 2H), 6.58 (t, J=7.6 Hz, 1H), 6.53 (d, J=8.0 Hz, 2H), 5.07 (s, 2H), 3.77 (s, 2H), 3.49-3.51 (m, 2H), 3.40-3.44 (m, 2H), 3.35-3.38 (m, 3H), 3.22-3.26 (m, 1H), 3.05-3.09 (m, 1H), 2.90 (s, 3H), 2.59-2.67 (m, 2H), 2.10-2.15 (m, 1H), 1.78-1.83 (m, 1H). LC-MS m/z: 565.3 [M+H]⁺. HPLC Purity (254 nm): 88.42%; t_{R}=9.582 min.

AC2020601-0437

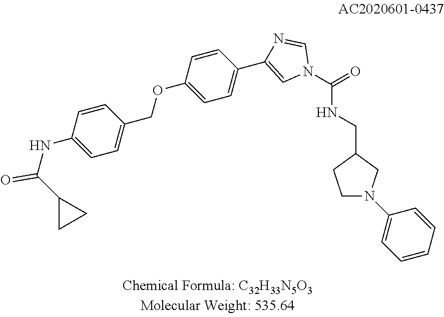 [SMILES: O=C(Nc1ccc(COc2ccc(-c3cn(C(=O)NCC4CCN(c5ccccc5)C4)cn3)cc2)cc1)C1CC1]

### The Synthesis of 4-(4-((4-(cyclopropanecarboxamido)benzyl)oxy)phenyl)-N-((1-phenylpyrrolidin-3-yl)methyl)-1H-imidazole-1-carboxamide (AC2020601-0437)

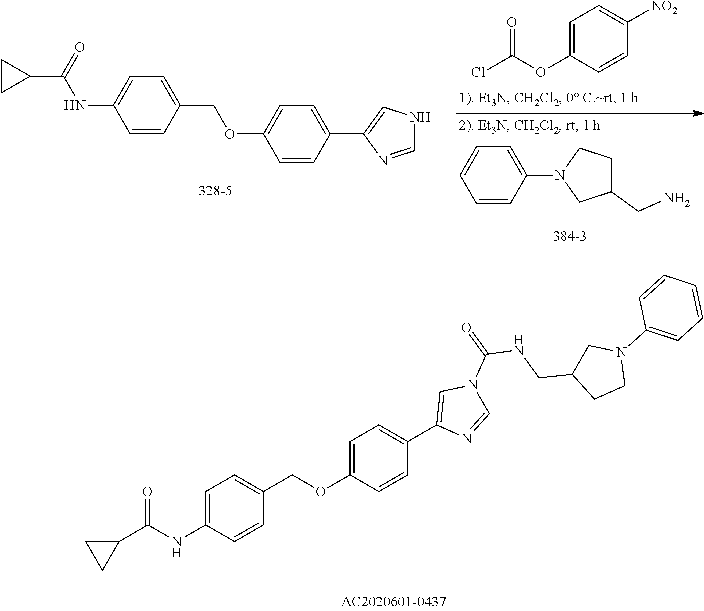 [SMILES: O=C(Nc1ccc(COc2ccc(-c3c[nH]cn3)cc2)cc1)C1CC1 ; NCC1CCN(c2ccccc2)C1 ; O=C(Cl)Oc1ccc([N+](=O)[O-])cc1 ; O=C(Nc1ccc(COc2ccc(-c3cn(C(=O)NCC4CCN(c5ccccc5)C4)cn3)cc2)cc1)C1CC1]

To a solution of 328-5 (110 mg, 0.33 mmol) in DCM (5 mL) was added 4-nitrophenyl carbonochloridate (80 mg, 0.40 mmol) and Et₃N (5 mL) at 0° C. and stirred for 1 hour. Then the reaction mixture was allowed to warm to room temperature and 384-3 (71 mg, 0.40 mmol) was added. After 1 hour stirring, the reaction mixture was quenched with water, extracted with DCM (20 mL), the combined organic layer was washed with brine, dried over anhydrous Na₂SO₄ and evaporated under reduced pressure. The crude was purified by silica gel chromatography (5% MeOH/DCM) to afford AC2020601-0437 (35 mg, yield: 19.8%) as a white solid. ¹H NMR (400 MHz, DMSO-d₆) δ 10.25 (s, 1H), 8.66 (t, J=5.6 Hz, 1H), 8.30 (d, J=1.2 Hz, 1H), 8.03 (d, J=0.8 Hz, 1H), 7.73 (d, J=8.4 Hz, 2H), 7.61 (d, J=8.4 Hz, 2H), 7.38 (d, J=8.4 Hz, 2H), 7.13-7.17 (m, 2H), 7.03 (d, J=8.8 Hz, 2H), 6.52-6.60 (m, 3H), 5.05 (s, 2H), 3.31-3.40 (m, 3H), 3.21-3.27 (m, 1H), 3.05-3.09 (m, 1H), 2.58-2.67 (m, 2H), 2.10-2.15 (m, 1H), 1.77-1.83 (m, 2H), 0.78-0.80 (m, 4H). LC-MS m/z: 558.3 [M+Na]⁺. HPLC Purity (254 nm): 97.31%; t_{R}=8.800 min.

AC2020601-0438

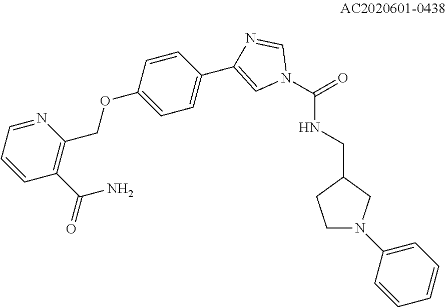 [SMILES: NC(=O)c1cccnc1COc1ccc(-c2cn(C(=O)NCC3CCN(c4ccccc4)C3)cn2)cc1]

### The Synthesis of tert-butyl 4-(4-((3-cyanopyridin-2-yl)methoxy)phenyl)-1H-imidazole-1-carboxylate (438-2)

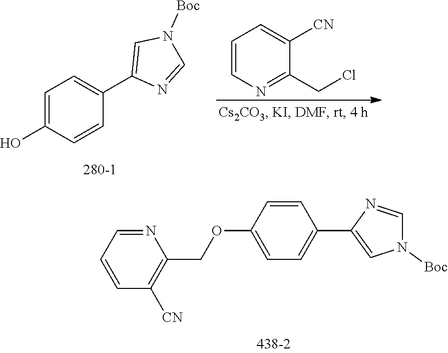 [SMILES: CC(C)(C)OC(=O)n1cnc(-c2ccc(O)cc2)c1 ; N#Cc1cccnc1CCl ; CC(C)(C)OC(=O)n1cnc(-c2ccc(OCc3ncccc3C#N)cc2)c1]

To a suspension of 280-1 (1.20 g, 4.61 mmol) in DMF (10 mL) was added Cs₂CO₃ (3.00 g, 9.22 mmol), KI (77 mg, 0.5 mmol) and 2-(chloromethyl)nicotinonitrile (703 mg, 4.61 mmol). The reaction mixture was stirred at room temperature for 4 hours until the reaction was complete (by LCMS). Saturated NaCl solution was added and the reaction mixture was extracted with EtOAc (20 mL), the combined organic layer was washed with brine, dried over anhydrous Na₂SO₄ and evaporated under reduced pressure. The crude was purified by silica gel chromatography (10% MeOH/DCM) to afford 438-2 (1.50 g, yield: 86%) as yellow oil.

### The Synthesis of 2-((4-(1H-imidazol-4-yl)phenoxy)methyl)nicotinonitrile (438-3)

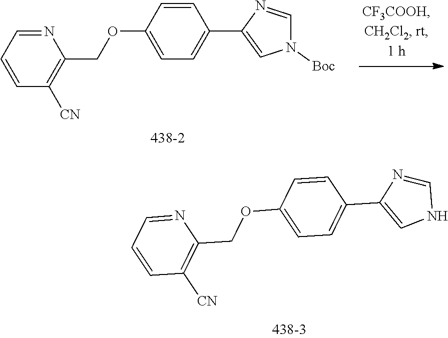 [SMILES: CC(C)(C)OC(=O)n1cnc(-c2ccc(OCc3ncccc3C#N)cc2)c1 ; N#Cc1cccnc1COc1ccc(-c2c[nH]cn2)cc1]

To a stirred solution of 438-2 (376 mg, 1.00 mmol) in CH₂Cl₂ (10 mL) was added TFA (5 mL). The reaction mixture was stirred at room temperature for 3 hours until the reaction was complete (by LCMS). The mixture was concentrated under reduced pressure to afford 438-3 (270 mg, crude) as yellow oil, which was used to the next step without purification.

### The Synthesis of 2-((4-(1H-imidazol-4-yl)phenoxy)methyl)nicotinamide (438-4)

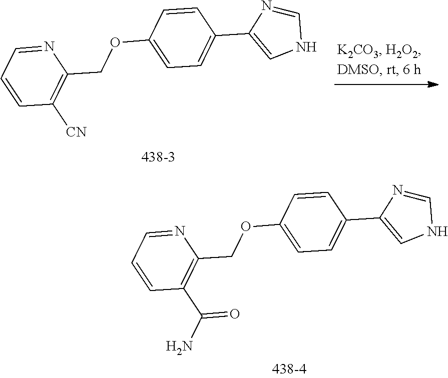 [SMILES: N#Cc1cccnc1COc1ccc(-c2c[nH]cn2)cc1 ; NC(=O)c1cccnc1COc1ccc(-c2c[nH]cn2)cc1]

To a suspension of 438-3 (270 mg, 0.99 mmol) in DMSO (10 mL) was added K₂CO₃ (276 mg, 2.00 mmol) and H₂O₂ (1 mL). The reaction mixture was stirred at room temperature for 6 hours until the reaction was complete (by LCMS). Saturated NaCl solution was added and the reaction mixture was extracted with EtOAc (20 mL), the combined organic layer was washed with brine, dried over anhydrous Na₂SO₄ and evaporated under reduced pressure. The crude was purified by silica gel chromatography (20% MeOH/DCM) to afford 438-4 (180 mg, yield: 6₂%) as yellow oil.

### The Synthesis of 2-((4-(1-(((1-phenylpyrrolidin-3-yl)methyl)carbamoyl)-1H-imidazol-4-yl)phenoxy)methyl)nicotinamide (AC2020601-0438)

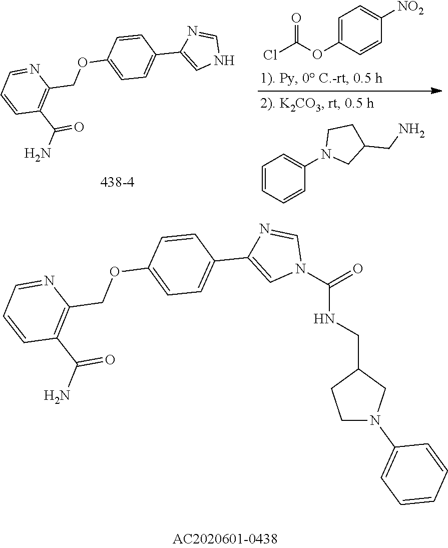

To a solution of 438-4 (97 mg, 0.33 mmol) in Py (5 mL) was added SM1 (82.0 mg, 0.4 mmol) at 0° C. and stirred for 1 hour. Then the reaction mixture was allowed to warm to room temperature and K₂CO₃ (138 mg, 1.0 mmol), 384-3 (71.0 mg, 0.4 mmol) was added. After 1 hour stirring, the reaction mixture was quenched with water, extracted with DCM (20 mL), the combined organic layer was washed with brine, dried over anhydrous Na₂SO₄ and evaporated under reduced pressure. The crude was purified by silica gel chromatography (10% MeOH/DCM) to afford AC2020601-0438 (35 mg, yield: 21%) as a white solid. ¹H NMR (400 MHz, DMSO-d₆) δ 8.61-8.67 (m, 2H), 8.30 (s, 1H), 8.02 (s, 2H), 7.89 (d, J=6.8 Hz, 1H), 7.71 (d, J=8.4 Hz, 2H), 7.62 (s, 1H), 7.45-7.48 (m, 1H), 7.13-7.17 (m, 2H), 7.01 (d, J=8.4 Hz, 2H), 6.52-6.60 (m, 3H), 5.34 (s, 2H), 3.34-3.40 (m, 4H), 3.23-3.27 (m, 1H), 3.05-3.09 (m, 1H), 2.61-2.64 (m, 1H), 2.10-2.15 (m, 1H), 1.78-1.83 (m, 1H). LC-MS m/z: 519.2 [M+Na]⁺. HPLC Purity (254 nm): 92.10%; t_{R}=7.013 min.

AC2020601-0439

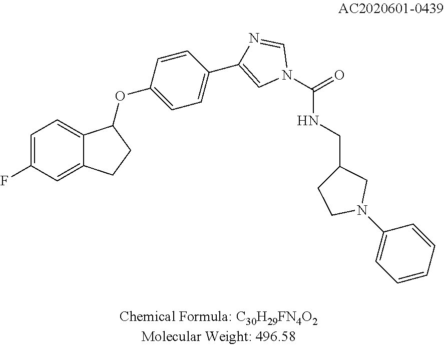 [SMILES: O=C(NCC1CCN(c2ccccc2)C1)n1cnc(-c2ccc(OC3CCc4cc(F)ccc43)cc2)c1]

### The Synthesis of tert-butyl 4-(4-((5-fluoro-2,3-dihydro-1H-inden-1-yl)oxy)phenyl)-1H-imidazole-1-carboxylate (439-2)

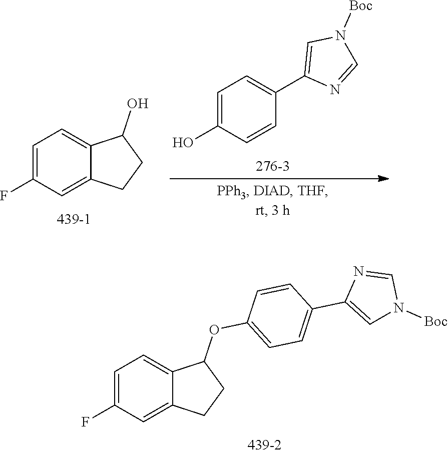 [SMILES: OC1CCc2cc(F)ccc21 ; CC(C)(C)OC(=O)n1cnc(-c2ccc(O)cc2)c1 ; CC(C)(C)OC(=O)n1cnc(-c2ccc(OC3CCc4cc(F)ccc43)cc2)c1]

To a suspension of 276-3 (780 mg, 3.0 mmol) in THF (10 mL) was added 5-fluoro-2,3-dihydro-1H-inden-1-ol (439-1, 507 mg, 3.0 mmol), PPh₃ (942.0 mg, 3.6 mmol) and DIAD (789.0 mg, 3.9 mmol). The reaction mixture was stirred at room temperature for 3 hours until the reaction was complete (by LCMS). Saturated NaCl solution was added and the reaction mixture was extracted with EtOAc (20 mL), the combined organic layer was washed with brine, dried over anhydrous Na₂SO₄ and evaporated under reduced pressure. The crude was purified by silica gel chromatography (10% MeOH/DCM) to afford 439-2 (665 mg, yield: 56.2%) as yellow oil.

### The Synthesis of 4-(4-((5-fluoro-2,3-dihydro-1H-inden-1-yl)oxy)phenyl)-1H-imidazole (439-3)

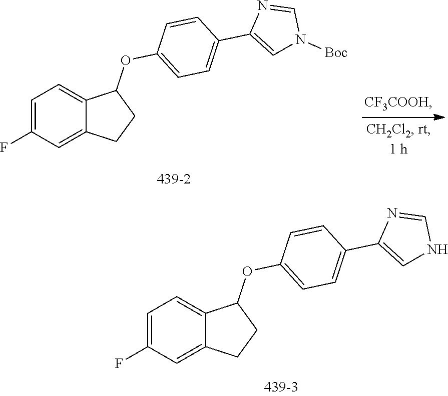 [SMILES: CC(C)(C)OC(=O)n1cnc(-c2ccc(OC3CCc4cc(F)ccc43)cc2)c1 ; Fc1ccc2c(c1)CCC2Oc1ccc(-c2c[nH]cn2)cc1]

To a stirred solution of 439-2 (394 mg, 1.00 mmol) in CH₂Cl₂ (10 mL) was added TFA (5 mL). The reaction mixture was stirred at room temperature for 1 hour until the reaction was complete (by LCMS). The mixture was concentrated under reduced pressure to afford 439-3 (290 mg, crude) as yellow oil, which was used to the next step without purification.

### The Synthesis of 4-(4-((5-fluoro-2,3-dihydro-1H-inden-1-yl)oxy)phenyl)-N-((1-phenylpyrrolidin-3-yl)methyl)-1H-imidazole-1-carboxamide (AC2020601-0439)

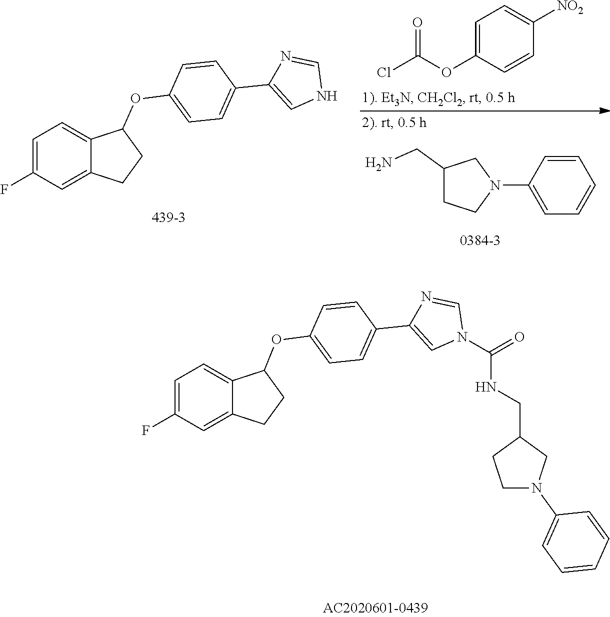 [SMILES: Fc1ccc2c(c1)CCC2Oc1ccc(-c2c[nH]cn2)cc1 ; NCC1CCN(c2ccccc2)C1 ; O=C(Cl)Oc1ccc([N+](=O)[O-])cc1 ; O=C(NCC1CCN(c2ccccc2)C1)n1cnc(-c2ccc(OC3CCc4cc(F)ccc43)cc2)c1]

To a solution of 439-3 (97 mg, 0.33 mmol) in DCM (5 mL) was added SM1 (82.0 mg, 0.4 mmol) and Et₃N (5 mL) at 0° C. and stirred for 0.5 hour. Then the reaction mixture was allowed to warm to room temperature and 384-3 (71.0 mg, 0.4 mmol) was added. After 0.5 hour stirring, the reaction mixture was quenched with water, extracted with DCM (20 mL), the combined organic layer was washed with brine, dried over anhydrous Na₂SO₄ and evaporated under reduced pressure. The crude was purified by silica gel chromatography (10% MeOH/DCM) to afford AC2020601-0439 (150 mg, yield: 91.5%) as a white solid. ¹H NMR (400 MHz, DMSO-d₆) δ 8.66 (t, J=5.2 Hz, 1H), 8.31 (d, J=0.8 Hz, 1H), 8.04 (s, 1H), 7.75 (d, J=8.4 Hz, 2H), 7.41-7.44 (m, 1H), 7.14-7.18 (m, 3H), 7.03-7.08 (m, 3H), 6.53-6.60 (m, 3H), 5.83-5.86 (m, 1H), 3.35-3.41 (m, 3H), 3.21-3.27 (m, 1H), 3.02-3.10 (m, 2H), 2.86-2.93 (m, 1H), 2.55-2.67 (m, 3H), 2.05-2.17 (m, 1H), 1.77-1.86 (m, 1H). LC-MS m/z: 495.1 [M+H]⁺. HPLC Purity (254 nm): 92.80%; t_{R}=11.155 min.

AC2020601-0443

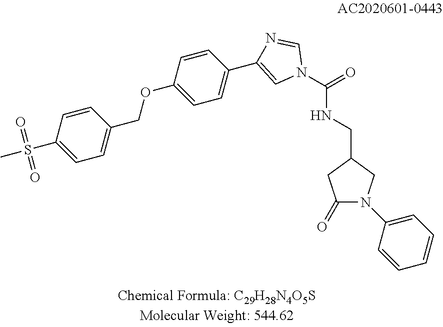 [SMILES: CS(=O)(=O)c1ccc(COc2ccc(-c3cn(C(=O)NCC4CC(=O)N(c5ccccc5)C4)cn3)cc2)cc1]

### The Synthesis of 4-(4-((4-(methylsulfonyl)benzyl)oxy)phenyl)-N-((5-oxo-1-phenylpyrrolidin-3-yl)methyl)-1H-imidazole-1-carboxamide (AC2020601-0443)

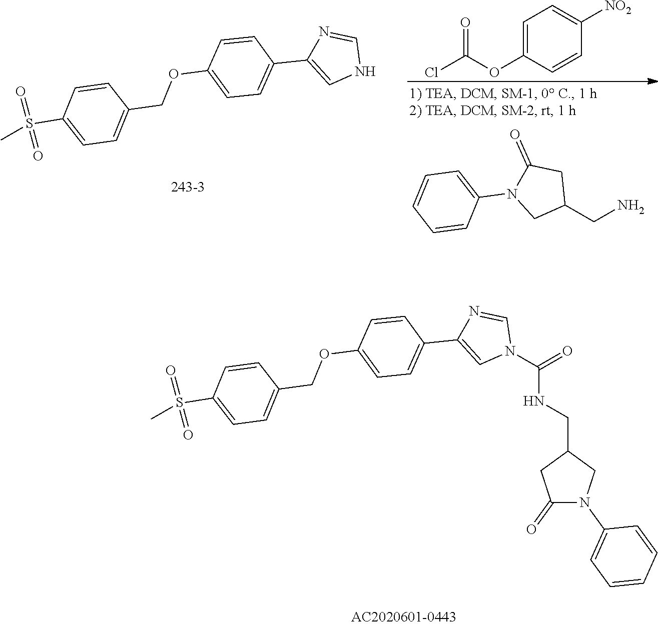 [SMILES: CS(=O)(=O)c1ccc(COc2ccc(-c3c[nH]cn3)cc2)cc1 ; NCC1CC(=O)N(c2ccccc2)C1 ; O=C(Cl)Oc1ccc([N+](=O)[O-])cc1 ; CS(=O)(=O)c1ccc(COc2ccc(-c3cn(C(=O)NCC4CC(=O)N(c5ccccc5)C4)cn3)cc2)cc1]

To a solution of 243-3 (100 mg, 0.30 mmol) in DCM (5 mL) was added 4-nitrophenyl carbonochloridate (68 mg, 0.33 mmol) and Et₃N (2 mL) at 0° C. and stirred for 1 hour. Then the reaction mixture was allowed to warm to room temperature and SM-2 (58 mg, 0.30 mmol) was added. After 1 hour stirring, the reaction mixture was quenched with water, extracted with DCM (20 mL), the combined organic layer was washed with brine, dried over anhydrous Na₂SO₄ and evaporated under reduced pressure. The crude was purified by silica gel chromatography (5% MeOH/DCM) to afford AC2020601-0443 (8 mg, yield: 4.8%) as a white solid. ¹H NMR (400 MHz, DMSO-d₆) δ 8.71 (t, J=5.6 Hz, 1H), 8.28 (d, J=1.2 Hz, 1H), 8.01 (d, J=0.8 Hz, 1H), 7.96 (d, J=8.4 Hz, 2H), 7.72-7.75 (m, 4H), 7.64-7.66 (m, 2H), 7.39 (m, 2H), 7.12-7.15 (m, 1H), 7.07 (d, J=8.8 Hz, 2H), 5.28 (s, 2H), 3.98-4.02 (m, 1H), 3.67-3.70 (m, 1H), 3.38-3.41 (m, 2H), 3.23 (s, 3H), 2.66-2.74 (m, 2H), 2.39-2.44 (m, 1H). LC-MS m/z: 545.3 [M+H]⁺. HPLC Purity (254 nm):96.18%; t_{R}=8.025 min.

AC2020601-0444

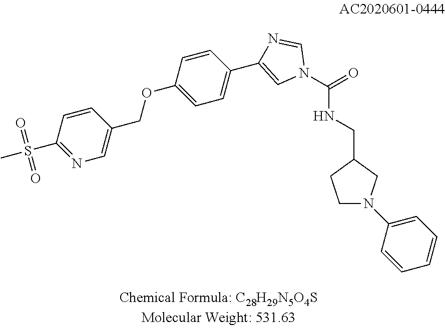 [SMILES: CS(=O)(=O)c1ccc(COc2ccc(-c3cn(C(=O)NCC4CCN(c5ccccc5)C4)cn3)cc2)cn1]

### The Synthesis of tert-butyl 4-(4-((6-(methylsulfonyl)pyridin-3-yl)methoxy)phenyl)-1H-imidazole-1-carboxylate (444-2)

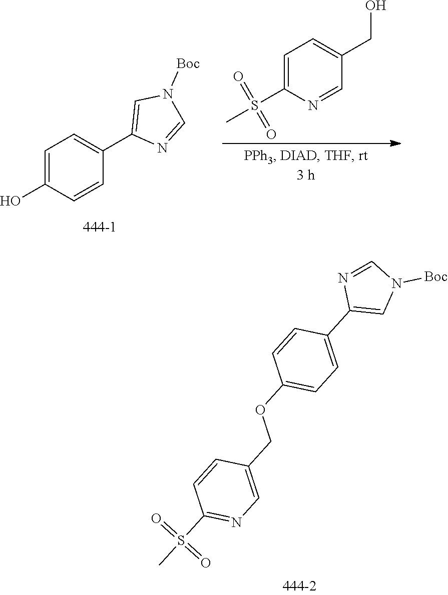

To a suspension of 444-1 (520 mg, 2.0 mmol) in THF (10 mL) was added (6-(methylsulfonyl)pyridin-3-yl)methanol (749 mg, 4.0 mmol), PPh₃ (629 mg, 2.4 mmol) and DIAD (525 mg, 2.6 mmol). The reaction mixture was stirred at room temperature for 3 hours until the reaction was complete (by LCMS). Water was added and the reaction mixture was extracted with EtOAc (50 mL), the combined organic layer was washed with brine, dried over anhydrous Na₂SO₄ and evaporated under reduced pressure. The crude was purified by silica gel chromatography (30% EA/PE) to afford 444-2 (400 mg, yield: 46%) as yellow oil.

### The Synthesis of 5-((4-(1H-imidazol-4-yl)phenoxy)methyl)-2-(methylsulfonyl)pyridine (444-3)

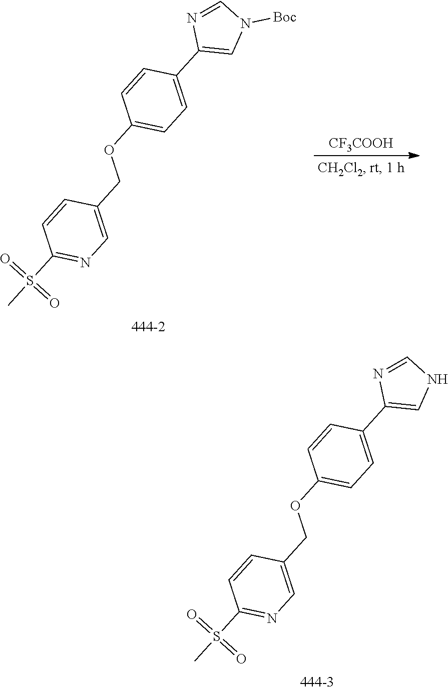 [SMILES: CC(C)(C)OC(=O)n1cnc(-c2ccc(OCc3ccc(S(C)(=O)=O)nc3)cc2)c1 ; "CF3COOH" ; CS(=O)(=O)c1ccc(COc2ccc(-c3c[nH]cn3)cc2)cn1]

To a stirred solution of 444-2 (400 mg, 0.9 mmol) in CH₂Cl₂ (10 mL) was added TFA (10 mL). The reaction mixture was stirred at room temperature for 1 hour until the reaction was complete (by LCMS). The reaction mixture was quenched with water, extracted with DCM (20 mL), the combined organic layer was washed with brine, dried over anhydrous Na₂SO₄. The mixture was concentrated under reduced pressure to afford 444-3 (280 mg, yield: 91%) as yellow oil, which was used to the next step without purification.

### The Synthesis of 4-(4-((6-(methylsulfonyl)pyridin-3-yl)methoxy)phenyl)-N-((1-phenylpyrrolidin-3-yl)methyl)-1H-imidazole-1-carboxamide (AC2020601-0444)

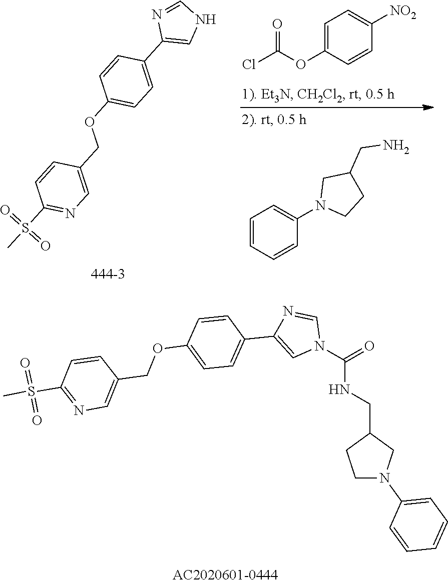 [SMILES: CS(=O)(=O)c1ccc(COc2ccc(-c3c[nH]cn3)cc2)cn1 ; NCC1CCN(c2ccccc2)C1 ; O=C(Cl)Oc1ccc([N+](=O)[O-])cc1 ; CS(=O)(=O)c1ccc(COc2ccc(-c3cn(C(=O)NCC4CCN(c5ccccc5)C4)cn3)cc2)cn1]

To a solution of 444-3 (280 mg, 0.85 mmol) in DCM (5 mL) was added 4-nitrophenyl carbonochloridate (261 mg, 1.3 mmol) and Et₃N (5 mL) at room temperature and stirred for 0.5 hour. Then (1-phenylpyrrolidin-3-yl)methanamine (300 mg, 1.7 mmol) was added. After 0.5 hour stirring, the reaction mixture was quenched with water, extracted with DCM (20 mL), the combined organic layer was washed with brine, dried over anhydrous Na₂SO₄ and evaporated under reduced pressure. The crude was purified by silica gel chromatography (5% MeOH/DCM) to afford AC2020601-0444 (31 mg, yield: 7.4%) as a white solid. ¹H NMR (400 MHz, DMSO-d₆) δ 8.66 (t, J=5.2 Hz, 1H), 8.51 (d, J=2.4 Hz, 1H), 8.30 (d, J=0.8 Hz, 1H), 8.04 (d, J=1.2 Hz, 1H), 7.85 (dd, J=8.4 Hz, J=2.8 Hz, 1H), 7.75 (d, J=8.8 Hz, 2H), 7.70 (d, J=8.0 Hz, 1H), 7.13-7.17 (m, 2H), 7.07 (d, J=8.8 Hz, 2H), 6.58 (t, J=7.2 Hz, 1H), 6.53 (d, J=8.0 Hz, 2H), 5.17 (s, 2H), 3.36-3.40 (m, 6H), 3.21-3.27 (m, 1H), 3.05-3.09 (m, 1H), 2.59-2.68 (m, 1H), 2.09-2.17 (m, 1H), 1.76-1.85 (m, 1H). LC-MS m/z: 554.0 [M+H]⁺. HPLC Purity (254 nm): 93.54%; t_{R}=8.551 min.

AC2020601-0445

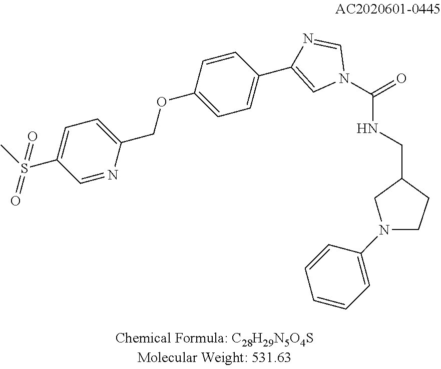 [SMILES: CS(=O)(=O)c1ccc(COc2ccc(-c3cn(C(=O)NCC4CCN(c5ccccc5)C4)cn3)cc2)nc1]

### The Synthesis of (5-(methylsulfonyl)pyridin-2-yl)methanol (445-2)

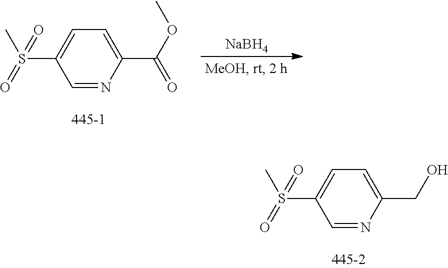

To a suspension of 445-1 (1.0 g, 4.6 mmol) in MeOH (10 mL) was added NaBH₄ (870 mg, 23.0 mmol). The reaction mixture was stirred at room temperature for 2 hours until the reaction was complete (by LCMS). Water was added and the reaction mixture was extracted with EtOAc (50 mL), the combined organic layer was washed with brine, dried over anhydrous Na₂SO₄ and evaporated under reduced pressure. The crude was purified by silica gel chromatography (30% EA/PE) to afford 445-2 (400 mg, yield: 44%) as white oil.

### The Synthesis of tert-butyl 4-(4-((5-(methylsulfonyl)pyridin-2-yl)methoxy)phenyl)-1H-imidazole-1-carboxylate (445-3)

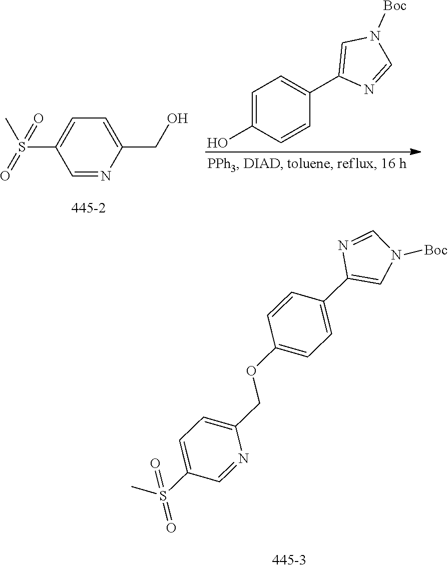 [SMILES: CS(=O)(=O)c1ccc(CO)nc1 ; CC(C)(C)OC(=O)n1cnc(-c2ccc(O)cc2)c1 ; CC(C)(C)OC(=O)n1cnc(-c2ccc(OCc3ccc(S(C)(=O)=O)cn3)cc2)c1]

To a suspension of 445-2 (400 mg, 2.1 mmol) in Tol (10 mL) was added tert-butyl 4-(4-hydroxyphenyl)-1H-imidazole-1-carboxylate (278 mg, 1.1 mmol), PPh₃ (335 mg, 1.3 mmol) and DIAD (280 mg, 1.4 mmol). The reaction mixture was allowed to warm to 110° C. for 16 hours until the reaction was complete (by LCMS). Water was added and the reaction mixture was extracted with EtOAc (50 mL), the combined organic layer was washed with brine, dried over anhydrous Na₂SO₄ and evaporated under reduced pressure. The crude was purified by silica gel chromatography (30% EA/PE) to afford 445-3 (400 mg, yield: 44%) as yellow oil.

### The Synthesis of 5-((4-(1H-imidazol-4-yl)phenoxy)methyl)-2-(methylsulfonyl)pyridine (445-4)

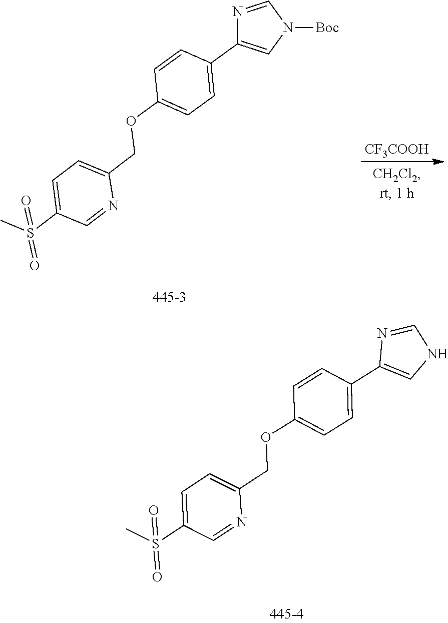 [SMILES: CC(C)(C)OC(=O)n1cnc(-c2ccc(OCc3ccc(S(C)(=O)=O)cn3)cc2)c1 ; "CF3COOH" ; CS(=O)(=O)c1ccc(COc2ccc(-c3c[nH]cn3)cc2)nc1]

To a stirred solution of 445-3 (400 mg, 0.9 mmol) in CH₂Cl₂ (10 mL) was added TFA (10 mL). The reaction mixture was stirred at room temperature for 1 hour until the reaction was complete (by LCMS). The reaction mixture was quenched with water, extracted with DCM (20 mL), the combined organic layer was washed with brine, dried over anhydrous Na₂SO₄. The mixture was concentrated under reduced pressure to afford 445-3 (200 mg, yield: 66%) as yellow oil, which was used to the next step without purification.

### The Synthesis of 4-(4-((5-(methylsulfonyl)pyridin-2-yl)methoxy)phenyl)-N-((1-phenylpyrrolidin-3-yl)methyl)-1H-imidazole-1-carboxamide (AC2020601-0445)

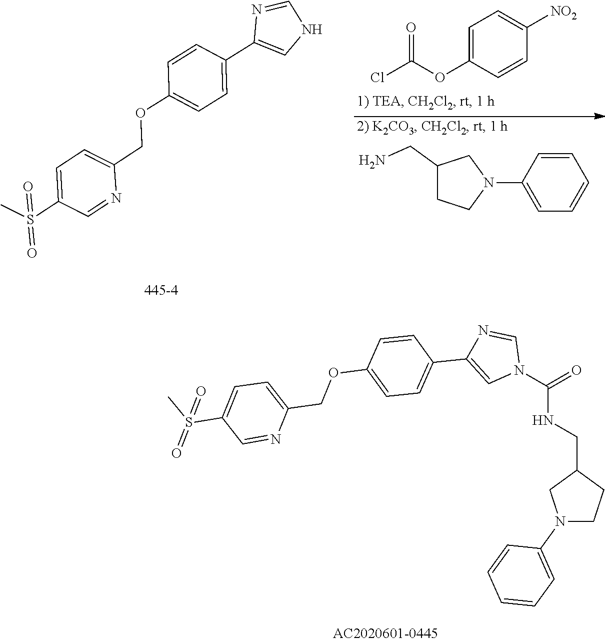 [SMILES: CS(=O)(=O)c1ccc(COc2ccc(-c3c[nH]cn3)cc2)nc1 ; NCC1CCN(c2ccccc2)C1 ; O=C(Cl)Oc1ccc([N+](=O)[O-])cc1 ; CS(=O)(=O)c1ccc(COc2ccc(-c3cn(C(=O)NCC4CCN(c5ccccc5)C4)cn3)cc2)nc1]

To a solution of 445-4 (200 mg, 0.6 mmol) in DCM (5 mL) was added 4-nitrophenyl carbonochloridate (180 mg, 0.9 mmol) and Et₃N (5 mL) at room temperature and stirred for 1 hour. Then (1-phenylpyrrolidin-3-yl)methanamine (211 mg, 1.2 mmol) and K₂CO₃ (165 mg, 1.2 mmol) was added. After 1 hour stirring, the reaction mixture was quenched with water, extracted with DCM (20 mL), the combined organic layer was washed with brine, dried over anhydrous Na₂SO₄ and evaporated under reduced pressure. The crude was purified by silica gel chromatography (5% MeOH/DCM) to afford AC2020601-0445 (7.56 mg, yield: 2.1%) as an off-white solid. ¹H NMR (400 MHz, DMSO-d₆) δ 9.09 (d, J=1.6 Hz, 1H), 8.67 (t, J=5.6 Hz, 1H), 8.38 (dd, J=8.4 Hz, J=2.4 Hz, 1H), 8.30 (d, J=1.2 Hz, 1H), 8.04 (d, J=0.8 Hz, 1H), 7.80 (d, J=8.4 Hz, 1H), 7.75 (d, J=8.8 Hz, 2H), 7.15 (t, J=7.2 Hz, 2H), 7.09 (d, J=9.2 Hz, 2H), 6.58 (t, J=7.2 Hz, 1H), 6.53 (d, J=8.0 Hz, 2H), 5.36 (s, 2H), 3.34-3.40 (m, 6H), 3.21-3.27 (m, 1H), 3.05-3.09 (m, 1H), 2.59-2.67 (m, 2H), 2.10-2.15 (m, 1H), 1.78-1.83 (m, 1H), LC-MS m/z: 532.3 [M+H]⁺. HPLC Purity (254 nm): 91.18%; t_{R}=3.653 min.

AC2020601-0447

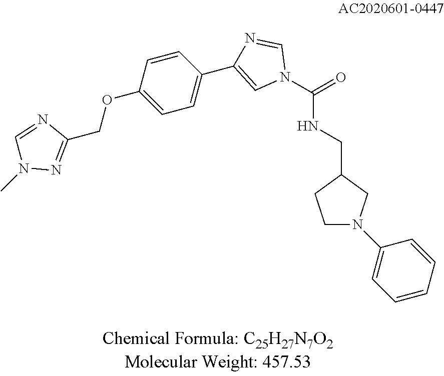 [SMILES: Cn1cnc(COc2ccc(-c3cn(C(=O)NCC4CCN(c5ccccc5)C4)cn3)cc2)n1]

### The Synthesis of tert-butyl 4-(4-((1-methyl-1H-1,2,4-triazol-3-yl)methoxy)phenyl)-1H-imidazole-1-carboxylate (447-2)

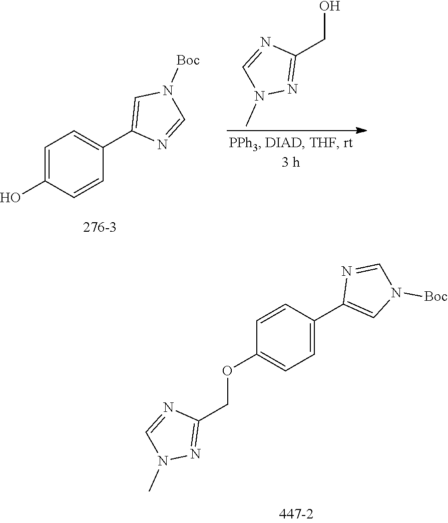

To a suspension of 276-3 (780 mg, 3.0 mmol) in THF (10 mL) was added (1-methyl-1H-1,2,4-triazol-3-yl)methanol (339 mg, 3.0 mmol), PPh₃ (942.0 mg, 3.6 mmol) and DIAD (789.0 mg, 3.9 mmol). The reaction mixture was stirred at room temperature for 3 hours until the reaction was complete (by LCMS). Saturated NaCl solution was added and the reaction mixture was extracted with EtOAc (20 mL), the combined organic layer was washed with brine, dried over anhydrous Na₂SO₄ and evaporated under reduced pressure. The crude was purified by silica gel chromatography (10% MeOH/DCM) to afford 447-2 (565 mg, yield: 53%) as yellow oil.

### The Synthesis of 3-((4-(1H-imidazol-4-yl)phenoxy)methyl)-1-methyl-1H-1,2,4-triazole (447-3)

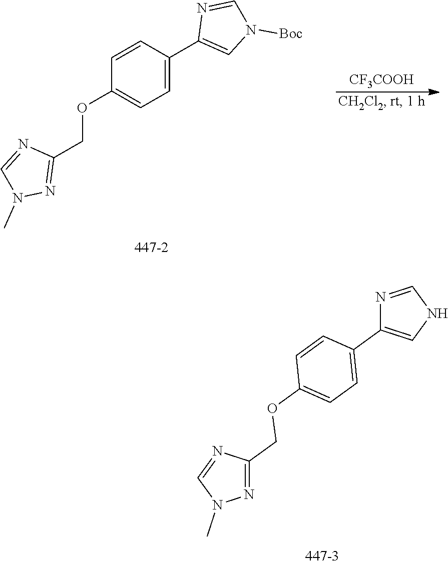 [SMILES: Cn1cnc(COc2ccc(-c3cn(C(=O)OC(C)(C)C)cn3)cc2)n1 ; "CF3COOH" ; "CH2Cl2, rt, 1 h" ; Cn1cnc(COc2ccc(-c3c[nH]cn3)cc2)n1]

To a stirred solution of 447-2 (355 mg, 1.00 mmol) in CH₂Cl₂ (10 mL) was added TFA (5 mL). The reaction mixture was stirred at room temperature for 1 hour until the reaction was complete (by LCMS). The mixture was concentrated under reduced pressure to afford 447-3 (250 mg, crude) as yellow oil, which was used to the next step without purification.

### The Synthesis of 4-(4-((1-methyl-1H-1,2,4-triazol-3-yl)methoxy)phenyl)-N-((1-phenylpyrrolidin-3-yl)methyl)-1H-imidazole-1-carboxamide (AC2020601-0447)

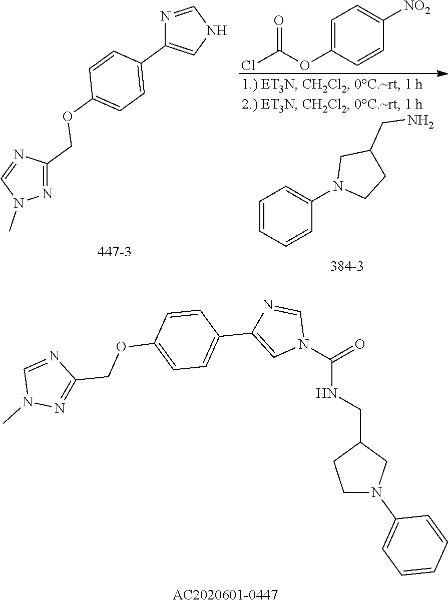 [SMILES: Cn1cnc(COc2ccc(-c3c[nH]cn3)cc2)n1 ; O=C(Cl)Oc1ccc([N+](=O)[O-])cc1 ; NCC1CCN(c2ccccc2)C1 ; Cn1cnc(COc2ccc(-c3cn(C(=O)NCC4CCN(c5ccccc5)C4)cn3)cc2)n1]

To a solution of 447-3 (102 mg, 0.4 mmol) in DCM (5 mL) was added SM1 (82.0 mg, 0.4 mmol) and Et₃N (5 mL) at 0° C. and stirred for 1 hour. Then the reaction mixture was allowed to warm to room temperature and 384-3 (71.0 mg, 0.4 mmol) was added. After 1 hour stirring, the reaction mixture was quenched with water, extracted with DCM (20 mL), the combined organic layer was washed with brine, dried over anhydrous Na₂SO₄ and evaporated under reduced pressure. The crude was purified by silica gel chromatography (10% MeOH/DCM) to afford AC2020601-0447 (6 mg, yield: 3.3%) as a white solid. ¹H NMR (400 MHz, DMSO-d₆) δ 8.66 (t, J=5.6 Hz, 1H), 8.48 (s, 1H), 8.30 (d, J=1.2 Hz, 1H), 8.03 (d, J=1.2 Hz, 1H), 7.73 (d, J=8.8 Hz, 2H), 7.13-7.17 (m, 2H), 7.06 (d, J=8.8 Hz, 2H), 6.52-6.60 (m, 3H), 5.07 (s, 2H), 3.87 (s, 3H), 3.31-3.40 (m, 4H), 3.21-3.27 (m, 1H), 3.05-3.09 (m, 1H), 2.59-2.65 (m, 1H), 2.07-2.17 (m, 1H), 1.77-1.85 (m, 1H). LC-MS m/z: 458.3 [M+H]⁺. HPLC Purity (254 nm): 95.72%; t_{R}=6.884 min.

AC2020601-0448

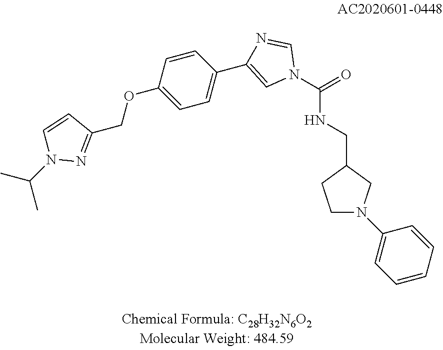 [SMILES: CC(C)n1ccc(COc2ccc(-c3cn(C(=O)NCC4CCN(c5ccccc5)C4)cn3)cc2)n1]

### The Synthesis of methyl 1-isopropyl-1H-pyrazole-3-carboxylate (448-1-2)

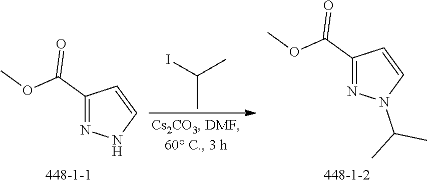

To a suspension of 448-1-1 (2.5 g, 20.0 mmol) in DMF (15 mL) was added 2-iodopropane (10.0 g, 60.0 mmol) and Cs₂CO₃ (13.0 g, 40.0 mmol). The reaction mixture was allowed to warm to 60° C. for 3 hours until the reaction was complete (by LCMS). Water was added and the reaction mixture was extracted with EtOAc (50 mL), the combined organic layer was washed with brine, dried over anhydrous Na₂SO₄ and evaporated under reduced pressure. The crude was purified by silica gel chromatography (15% PE/EA) to afford 448-1-2 (1.2 g, yield: 37.1%) as white oil.

### The Synthesis of (1-isopropyl-1H-pyrazol-3-yl)methanol (448-1-3)

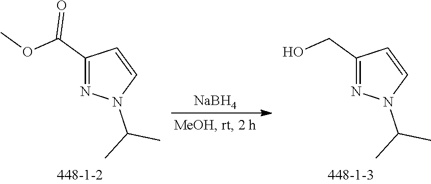

To a suspension of 448-1-2 (1.2 g, 7.1 mmol) in MeOH (20 mL) was added NaBH₄ (1.3 g, 35.5 mmol). The reaction mixture was stirred at room temperature for 2 hours until the reaction was complete (by LCMS). Water was added and the reaction mixture was extracted with EtOAc (50 mL), the combined organic layer was washed with brine, dried over anhydrous Na₂SO₄ and evaporated under reduced pressure. The crude was purified by silica gel chromatography (15% PE/EA) to afford 448-1-3 (800 mg, yield: 77.2%) as white oil.

### The Synthesis of tert-butyl 4-(4-((1-isopropyl-1H-pyrazol-3-yl)methoxy)phenyl)-1H-imidazole-1-carboxylate (448-2)

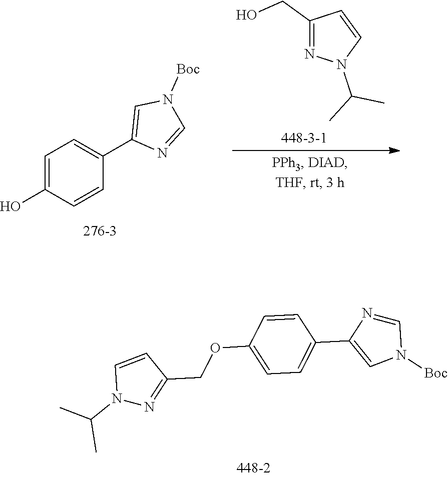

To a suspension of 448-1-3 (800 mg, 4.75 mmol) in THF (20 mL) was added 276-3 (618 mg, 2.38 mmol), PPh₃ (746 mg, 2.85 mmol) and DIAD (626 mg, 3.1 mmol). The reaction mixture was stirred at room temperature for 3 hours until the reaction was complete (by LCMS). Water was added and the reaction mixture was extracted with EtOAc (50 mL), the combined organic layer was washed with brine, dried over anhydrous Na₂SO₄ and evaporated under reduced pressure. The crude was purified by silica gel chromatography (45% EA/PE) to afford 448-2 (500 mg, yield: 22.4%) as yellow oil.

### The Synthesis of 3-((4-(1H-imidazol-4-yl)phenoxy)methyl)-1-isopropyl-1H-pyrazole (448-3)

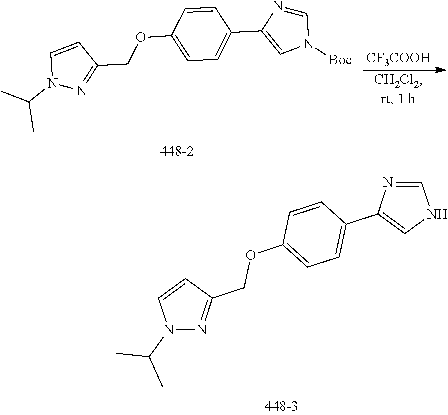 [SMILES: CC(C)n1ccc(COc2ccc(-c3cn(C(=O)OC(C)(C)C)cn3)cc2)n1 ; "CF3COOH" ; "CH2Cl2,rt, 1 h" ; CC(C)n1ccc(COc2ccc(-c3c[nH]cn3)cc2)n1]

To a stirred solution of 448-2 (500 mg, 1.3 mmol) in CH₂Cl₂ (10 mL) was added TFA (10 mL). The reaction mixture was stirred at room temperature for 1 hour until the reaction was complete (by LCMS). The reaction mixture was quenched with water, extracted with DCM (20 mL), the combined organic layer was washed with brine, dried over anhydrous Na₂SO₄. The mixture was concentrated under reduced pressure to afford 448-3 (250 mg, yield: 67.6%) as yellow oil, which was used to the next step without purification.

### The Synthesis of 4-(4-((1-isopropyl-1H-pyrazol-3-yl)methoxy)phenyl)-N-((1-phenylpyrrolidin-3-yl)methyl)-1H-imidazole-1-carboxamide (AC2020601-0448)

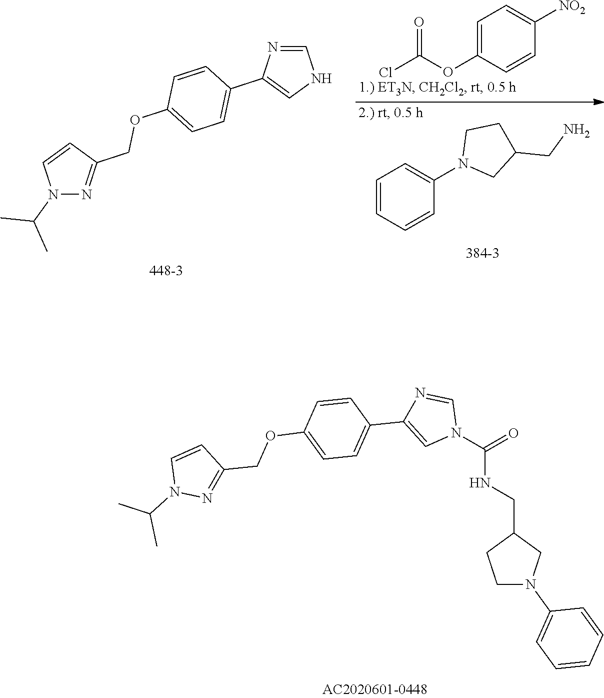 [SMILES: CC(C)n1ccc(COc2ccc(-c3c[nH]cn3)cc2)n1 ; NCC1CCN(c2ccccc2)C1 ; O=C(Cl)Oc1ccc([N+](=O)[O-])cc1 ; CC(C)n1ccc(COc2ccc(-c3cn(C(=O)NCC4CCN(c5ccccc5)C4)cn3)cc2)n1]

To a solution of 448-3 (250 mg, 0.89 mmol) in DCM (5 mL) was added 4-nitrophenyl carbonochloridate (269 mg, 1.34 mmol) and Et₃N (5 mL) at room temperature and stirred for 0.5 hour. Then (1-phenylpyrrolidin-3-yl)methanamine (384-3, 288 mg, 1.78 mmol) was added. After 0.5 hour stirring, the reaction mixture was quenched with water, extracted with DCM (20 mL), the combined organic layer was washed with brine, dried over anhydrous Na₂SO₄ and evaporated under reduced pressure. The crude was purified by silica gel chromatography (5% MeOH/DCM) to afford AC2020601-0448 (18 mg, yield: 4%) as a white solid. ¹H NMR (400 MHz, DMSO-d₆) δ 8.65 (t, J=5.2 Hz, 1H), 8.29 (s, 1H), 8.03 (s, 1H), 7.71-7.75 (m, 3H), 7.15 (t, J=8.0 Hz, 2H), 7.05 (d, J=8.4 Hz, 2H), 6.58 (t, J=7.2 Hz, 1H), 6.53 (d, J=8.4 Hz, 2H), 6.31 (d, J=2.0 Hz, 1H), 5.01 (s, 2H), 4.49 (t, J=6.4 Hz, 1H), 3.36-3.40 (m, 4H), 3.23-3.26 (m, 1H), 3.05-3.09 (m, 1H), 2.61-2.64 (m, 1H), 2.10-2.13 (m, 1H), 1.78-1.83 (m, 1H), 1.41 (d, J=6.8 Hz, 6H). LC-MS m/z: 485.5 [M+H]⁺. HPLC Purity (254 nm): 93.67%; t_{R}=8.241 min.

AC2020601-0449

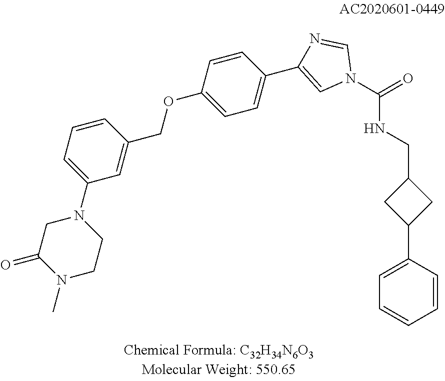 [SMILES: CN1CCN(c2cccc(COc3ccc(-c4cn(C(=O)NCC5CC(c6ccccc6)C5)cn4)cc3)c2)CC1=O]

### The Synthesis of 4-(4-(3-(4-methyl-3-oxopiperazin-1-yl)benzyloxy)phenyl)-N-((1-phenylazetidin-3-yl)methyl)-1H-imidazole-1-carboxamide (AC2020601-0449)

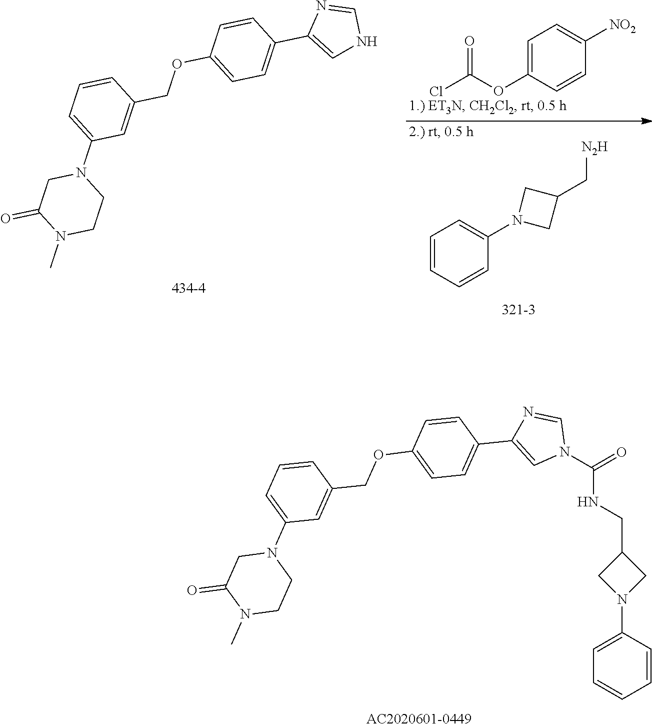

To a solution of 434-4 (150 mg, 0.41 mmol) in DCM (5 mL) was added 4-nitrophenyl carbonochloridate (123 mg, 0.61 mmol) and Et₃N (5 mL) at room temperature and stirred for 0.5 hour. Then 3-(diazenylmethyl)-1-phenylazetidine (321-3, 133 mg, 0.82 mmol) was added. After 0.5 hour stirring, the reaction mixture was quenched with water, extracted with DCM (20 mL), the combined organic layer was washed with brine, dried over anhydrous Na₂SO₄ and evaporated under reduced pressure. The crude was purified by silica gel chromatography (5% MeOH/DCM) to afford AC2020601-0449 (32.0 mg, yield: 14.2%) as a white solid. ¹H NMR (400 MHz, DMSO-d₆) δ 8.68 (t, J=5.6 Hz, 1H), 8.27 (d, J=1.2 Hz, 1H), 8.00 (d, J=1.2 Hz, 1H), 7.72 (dd, J=6.8 Hz, J=2.0 Hz, 2H), 7.25 (t, J=8.0 Hz, 1H), 7.16 (dd, J=8.4 Hz, J=7.6 Hz, 2H), 7.03-7.05 (m, 3H), 6.88-6.92 (m, 2H), 6.66 (t, J=7.2 Hz, 1H), 6.38-6.43 (m, 2H), 5.07 (s, 2H), 3.88 (t, J=7.6 Hz, 2H), 3.77 (s, 2H), 3.55-3.60 (m, 4H), 3.48-3.52 (m, 2H), 3.41-3.46 (m, 2H), 2.91-2.95 (m, 1H), 2.89 (s, 3H). LC-MS m/z: 551.3 [M+H]⁺. HPLC Purity (254 nm): 93.80%; t_{R}=9.035 min.

AC2020601-0451

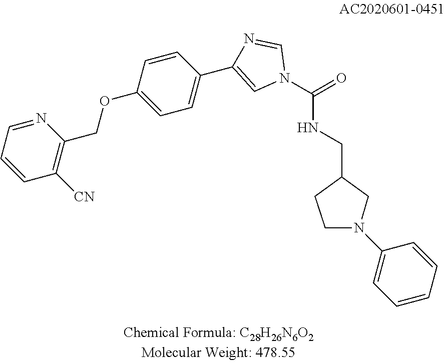 [SMILES: N#Cc1cccnc1COc1ccc(-c2cn(C(=O)NCC3CCN(c4ccccc4)C3)cn2)cc1]

### The Synthesis of 4-(4-((3-cyanopyridin-2-yl)methoxy)phenyl)-N-((1-phenylpyrrolidin-3-yl)methyl)-1H-imidazole-1-carboxamide (AC2020601-0451)

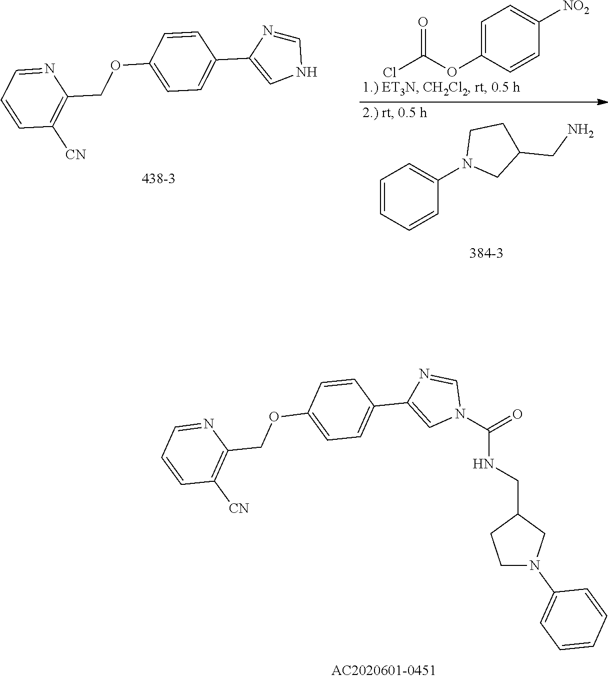 [SMILES: N#Cc1cccnc1COc1ccc(-c2c[nH]cn2)cc1 ; NCC1CCN(c2ccccc2)C1 ; O=C(Cl)Oc1ccc([N+](=O)[O-])cc1 ; N#Cc1cccnc1COc1ccc(-c2cn(C(=O)NCC3CCN(c4ccccc4)C3)cn2)cc1]

To a solution of 438-3 (100 mg, 0.36 mmol) in DCM (5 mL) was added 4-nitrophenyl carbonochloridate (108 mg, 0.54 mmol) and Et₃N (5 mL) at room temperature and stirred for 0.5 hour. Then (1-phenylpyrrolidin-3-yl)methanamine (384-3, 126 mg, 0.74 mmol) was added. After 0.5 hour stirring, the reaction mixture was quenched with water, extracted with DCM (20 mL), the combined organic layer was washed with brine, dried over anhydrous Na₂SO₄ and evaporated under reduced pressure. The crude was purified by silica gel chromatography (5% MeOH/DCM) to afford AC2020601-0451 (32.0 mg, yield: 14.2%) as a yellow solid. ¹H NMR (400 MHz, DMSO-d₆) δ 8.86 (dd, J=4.8 Hz, J=1.6 Hz, 1H), 8.66 (t, J=4.8 Hz, 1H), 8.41 (dd, J=7.6 Hz, J=1.6 Hz, 1H), 8.30 (d, J=1.2 Hz, 1H), 8.04 (d, J=1.2 Hz, 1H), 7.75 (d, J=8.4 Hz, 2H), 7.64 (dd, J=8.0 Hz, J=4.8 Hz, 1H), 7.15 (dd, J=8.4 Hz, J=7.2 Hz, 2H), 7.09 (d, J=8.8 Hz, 2H), 6.58 (t, J=7.6 Hz, 1H), 6.53 (d, J=7.6 Hz, 2H), 5.36 (s, 2H), 3.21-3.29 (m, 1H), 3.05-3.09 (m, 1H), 2.58-2.73 (m, 5H), 2.09-2.15 (m, 1H), 1.78-1.83 (m, 1H). LC-MS m/z: 479.3 [M+H]⁺. HPLC Purity (254 nm): 65.70%; t_{R}=9.506 min.

AC2020601-0452

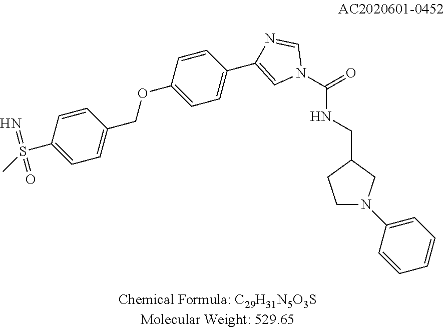 [SMILES: CS(=N)(=O)c1ccc(COc2ccc(-c3cn(C(=O)NCC4CCN(c5ccccc5)C4)cn3)cc2)cc1]

### The Synthesis of methyl(4-((trityloxy)methyl)phenyl)sulfane (452-2)

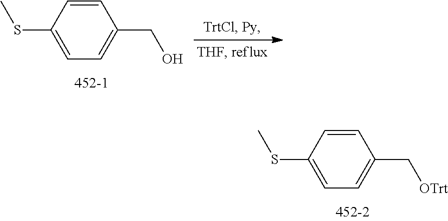

To a suspension of 452-1 (15.4 g, 99.85 mmol) in THF (150 mL) was added TrtCl (33.4 g, 119.82 mmol) and Py (15.8 g, 199.70 mmol). The reaction mixture was allowed to warm to 60° C. for 3 hours until the reaction was complete (by LCMS). Water was added and the reaction mixture was extracted with EtOAc (250 mL), the combined organic layer was washed with brine, dried over anhydrous Na₂SO₄ and evaporated under reduced pressure. The crude was purified by silica gel chromatography (15% PE/EA) to afford 452-2 (33 g, yield: 83.3%) as white oil.

### The Synthesis of (((4-(S-methylsulfonimidoyl)benzyl)oxy)methanetriyl)tribenzene (452-3)

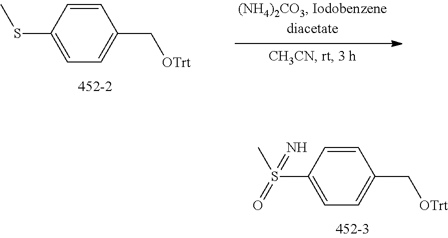

To a suspension of 452-2 (33 g, 83.22 mmol) in CH₃CN (200 mL) was added (NH₄)₂CO₃ (39.98 g, 416.10 mmol) and Iodobenzene diacetate (80.41 g, 249.66 mmol). The reaction mixture was stirred at room temperature for 3 hours until the reaction was complete (by LCMS). Water was added and the reaction mixture was extracted with EtOAc (250 mL), the combined organic layer was washed with brine, dried over anhydrous Na₂SO₄ and evaporated under reduced pressure. The crude was purified by silica gel chromatography (47% PE/EA) to afford 452-3 (20 g, yield: 56.2%) as white oil.

### The Synthesis of (9H-fluoren-9-yl)methyl (methyl(oxo)(4-((trityloxy)methyl)phenyl)-16-sulfaneylidene)carbamate (452-4)

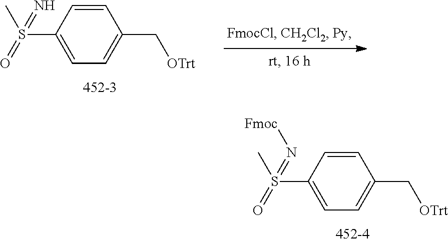

To a suspension of 452-3 (20.0 g, 46.78 mmol) in CH₂Cl₂ (150 mL) was added FmocCl (14.52 g, 56.13 mmol) and Py (7.40 g, 93.55 mmol). The reaction mixture was allowed to room temperature for 16 hours until the reaction was complete (by LCMS). Water was added and the reaction mixture was extracted with CH₂Cl₂ (250 mL), the combined organic layer was washed with brine, dried over anhydrous Na₂SO₄ and evaporated under reduced pressure. The crude was purified by silica gel chromatography (35% PE/EA) to afford 452-4 (23 g, yield: 75.7%) as a white solid.

### The Synthesis of (9H-fluoren-9-yl)methyl ((4-(hydroxymethyl)phenyl)(methyl)(oxo)-16-sulfaneylidene)carbamate (452-5)

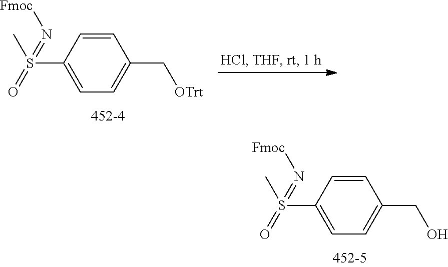

To a stirred solution of 452-4 (650 mg, 1.00 mmol) in THF (10 mL) was added HCl (1 mL, 4N). The reaction mixture was stirred at room temperature for 1 hour until the reaction was complete (by LCMS). The mixture was concentrated under reduced pressure to afford 452-5 (400 mg, crude) as yellow oil, which was used to the next step without purification.

### The Synthesis of tert-butyl 4-(4-((4-(N-(((9H-fluoren-9-yl)methoxy)carbonyl)-S-methylsulfonimidoyl)benzyl)oxy)phenyl)-1H-imidazole-1-carboxylate (452-6)

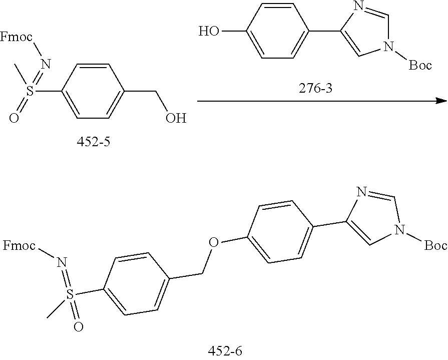

To a suspension of 452-5 (1.94 g, 4.75 mmol) in THF (20 mL) was added 276-3 (618 mg, 2.38 mmol), PPh₃ (746 mg, 2.85 mmol) and DIAD (626 mg, 3.10 mmol). The reaction mixture was stirred at room temperature for 3 hours until the reaction was complete (by LCMS). Water was added and the reaction mixture was extracted with EtOAc (50 mL), the combined organic layer was washed with brine, dried over anhydrous Na₂SO₄ and evaporated under reduced pressure. The crude was purified by silica gel chromatography (55% EA/PE) to afford 452-6 (1.01 g, yield: 65.3%) as yellow oil.

### The Synthesis of (9H-fluoren-9-yl)methyl ((4-((4-(1H-imidazol-4-yl)phenoxy)methyl)phenyl)(methyl)(oxo)-16-sulfaneylidene)carbamate (452-7)

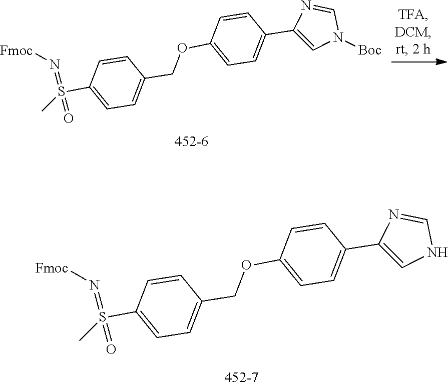

To a stirred solution of 452-6 (845 mg, 1.30 mmol) in CH₂Cl₂ (3 mL) was added TFA (3 mL). The reaction mixture was stirred at room temperature for 2 hours until the reaction was complete (by LCMS). The mixture was concentrated under reduced pressure to afford 452-7 (700 mg, crude) as yellow oil, which was used to the next step without purification.

### The Synthesis of (9H-fluoren-9-yl)methyl (methyl(oxo)(4-((4-(1-(((1-phenylpyrrolidin-3-yl)methyl)carbamoyl)-1H-imidazol-4-yl)phenoxy)methyl)phenyl)-16-sulfaneylidene)carbamate (452-8)

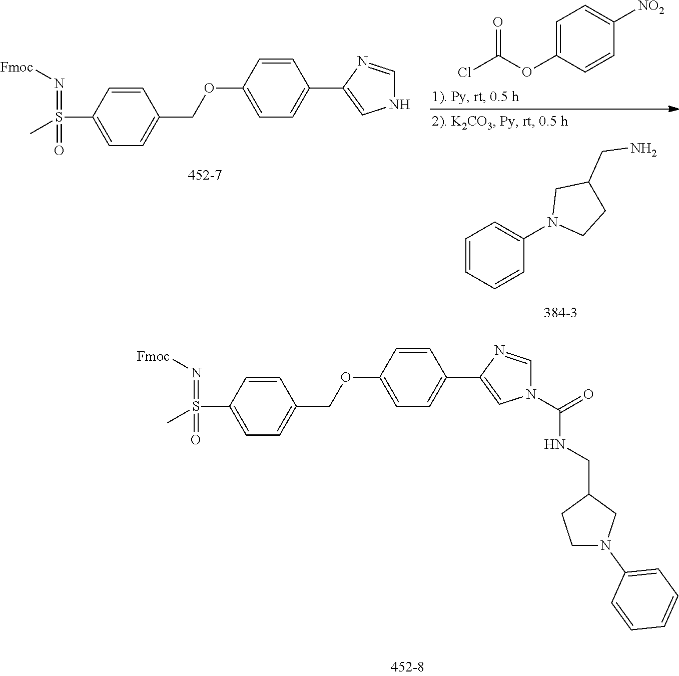

To a solution of 452-7 (550 mg, 0.99 mmol) in Py (5 mL) was added 4-nitrophenyl carbonochloridate (302 mg, 1.50 mmol) at room temperature and stirred for 0.5 hour. Then K₂CO₃ (276 mg, 2.00 mmol) and (1-phenylpyrrolidin-3-yl)methanamine (384-3, 353 mg, 2.00 mmol) was added. After 0.5 hour stirring, the reaction mixture was quenched with water, extracted with EtOAc (20 mL), the combined organic layer was washed with brine, dried over anhydrous Na₂SO₄ and evaporated under reduced pressure. The crude was purified by silica gel chromatography (5% MeOH/DCM) to afford 452-8 (300 mg, yield: 40.3%) as a white solid.

### The Synthesis of 4-(4-((4-(S-methylsulfonimidoyl)benzyl)oxy)phenyl)-N-((1-phenylpyrrolidin-3-yl)methyl)-1H-imidazole-1-carboxamide (AC2020601-0452)

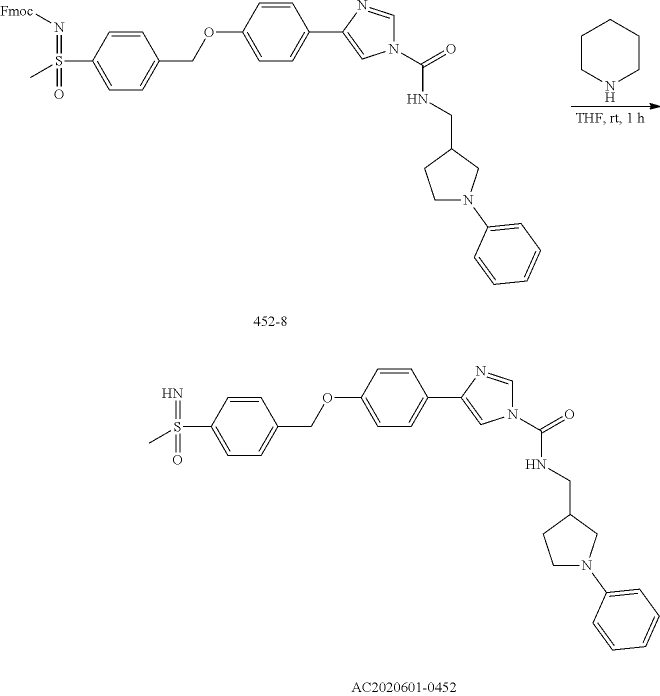

To a suspension of 452-8 (300 mg, 0.40 mmol) in THF (10 mL) was added piperidine (68 mg, 0.80 mmol). The reaction mixture was stirred at room temperature for 1 hour until the reaction was complete (by LCMS). Water was added and the reaction mixture was extracted with EtOAc (50 mL), the combined organic layer was washed with brine, dried over anhydrous Na₂SO₄ and evaporated under reduced pressure. The crude was purified by Prep-HPLC to afford AC2020601-0452 (142 mg, yield: 67.2%) as a white solid. ¹H NMR (400 MHz, DMSO-d₆) δ 8.66 (t, J=5.2 Hz, 1H), 8.30 (s, 1H), 8.04 (s, 1H), 7.96 (d, J=8.4 Hz, 2H), 7.75 (d, J=8.8 Hz, 2H), 7.67 (d, J=8.4 Hz, 2H), 7.13-7.17 (m, 2H), 7.07 (d, J=8.8 Hz, 2H), 6.52-6.60 (m, 3H), 5.26 (s, 2H), 4.28 (brs, 1H), 3.31-3.34 (m, 4H), 3.20-3.26 (m, 1H), 3.05-3.09 (m, 4H), 2.59-2.66 (m, 1H), 2.08-2.16 (m, 1H), 1.76-1.85 (m, 1H). LC-MS m/z: 530.2 [M+H]⁺. HPLC Purity (254 nm): 100%; t_{R}=3.645 min.

AC2020601-0456

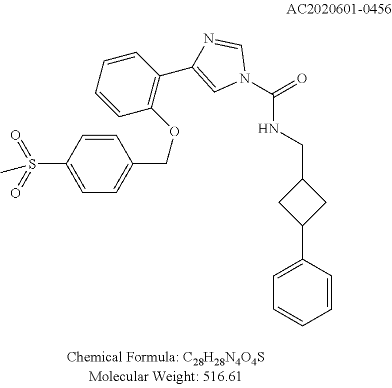 [SMILES: CS(=O)(=O)c1ccc(COc2ccccc2-c2cn(C(=O)NCC3CC(c4ccccc4)C3)cn2)cc1]

### The Synthesis of 2-(1H-imidazol-4-yl)phenol (456-2)

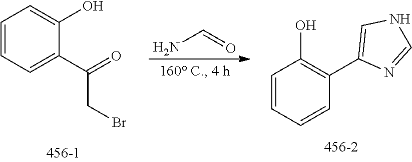

To a suspension of 456-1 (2.2 g, 4.8 mmol) in formamide (15 mL). The reaction mixture was allowed to warm to 160° C. for 4 hours until the reaction was complete (by LCMS). The crude was afford 456-2 (2.0 g, yield: 97.4%), which was used to the next step without purification.

### The Synthesis of tert-butyl 4-(2-hydroxyphenyl)-1H-imidazole-1-carboxylate (456-3)

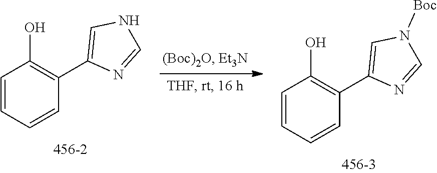 [SMILES: Oc1ccccc1-c1c[nH]cn1 ; CC(C)(C)OC(=O)n1cnc(-c2ccccc2O)c1]

To a suspension of 456-2 (2.0 g, 12.5 mmol) in THF (20 mL) was added Et₃N (10 mL) and (Boc)₂O (4.1 g, 18.7 mmol), The reaction mixture was stirred at room temperature for 16 hours until the reaction was complete (by LCMS). Water was added and the reaction mixture was extracted with EtOAc (50 mL), the combined organic layer was washed with brine, dried over anhydrous Na₂SO₄ and evaporated under reduced pressure. The crude was purified by silica gel chromatography (15% MeOH/DCM) to afford 456-3 (2.5 g, yield: 77%) as brown oil.

### The Synthesis of tert-butyl 4-(2-(4-(methylsulfonyl)benzyloxy)phenyl)-1H-imidazole-1-carboxylate (456-4)

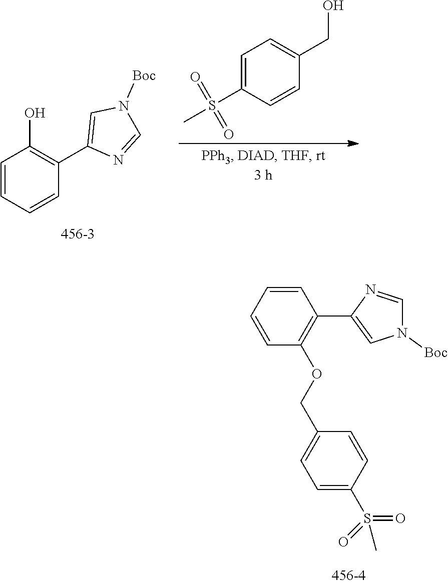

To a suspension of 456-3 (2.5 g, 9.6 mmol) in THF (20 mL) was added (4-(methylsulfonyl)phenyl)methanol (3.58 g, 19.2 mmol), PPh₃ (3.0 g, 11.5 mmol) and DIAD (2.5 g, 12.5 mmol). The reaction mixture was stirred at room temperature for 3 hours until the reaction was complete (by LCMS). Water was added and the reaction mixture was extracted with EtOAc (50 mL), the combined organic layer was washed with brine, dried over anhydrous Na₂SO₄ and evaporated under reduced pressure. The crude was purified by silica gel chromatography (45% EA/PE) to afford 456-4 (1.2 g, yield: 29%) as yellow oil.

### The Synthesis of 4-(2-(4-(methylsulfonyl)benzyloxy)phenyl)-1H-imidazole (456-5)

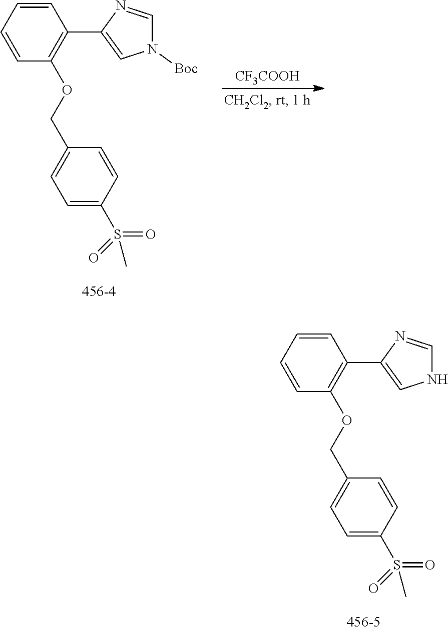 [SMILES: CC(C)(C)OC(=O)n1cnc(-c2ccccc2OCc2ccc(S(C)(=O)=O)cc2)c1 ; "CF3COOH" ; CS(=O)(=O)c1ccc(COc2ccccc2-c2c[nH]cn2)cc1]

To a stirred solution of 456-4 (1.2 g, 2.8 mmol) in CH₂Cl₂ (10 mL) was added TFA (10 mL). The reaction mixture was stirred at room temperature for 1 hour until the reaction was complete (by LCMS). The reaction mixture was quenched with water, extracted with DCM (20 mL), the combined organic layer was washed with brine, dried over anhydrous Na₂SO₄. The mixture was concentrated under reduced pressure to afford 456-5 (600 mg, yield: 65%) as yellow oil, which was used to the next step without purification.

### The Synthesis of 4-(2-(4-(methylsulfonyl)benzyloxy)phenyl)-N-((1-phenylazetidin-3-yl)methyl)-1H-imidazole-1-carboxamide (AC2020601-0456)

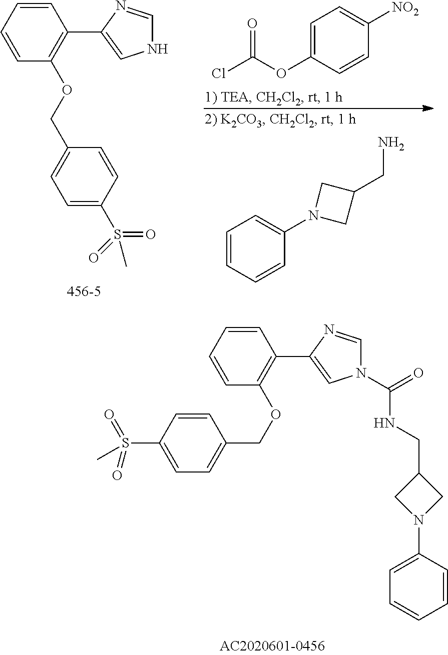

To a solution of 456-5 (200 mg, 0.6 mmol) in DCM (5 mL) was added 4-nitrophenyl carbonochloridate (180 mg, 0.9 mmol) and Et₃N (5 mL) at room temperature and stirred for 1 hour. Then (1-phenylazetidin-3-yl)methanamine (194 mg, 1.2 mmol) and K₂CO₃ (165 mg, 1.2 mmol) was added. After 1 hour stirring, the reaction mixture was quenched with water, extracted with DCM (20 mL), the combined organic layer was washed with brine, dried over anhydrous Na₂SO₄ and evaporated under reduced pressure. The crude was purified by silica gel chromatography (5% MeOH/DCM) to afford AC2020601-0456 (5.89 mg, yield: 1.8%) as white oil. ¹H NMR (400 MHz, DMSO-d₆) δ 8.77 (t, J=5.6 Hz, 1H), 8.35 (d, J=1.2 Hz, 1H), 8.13 (d, J=2.0 Hz, 1H), 8.11 (d, J=0.8 Hz, 1H), 7.95 (d, J=8.4 Hz, 2H), 7.74 (d, J=8.4 Hz, 2H), 7.01-7.24 (m, 5H), 6.65 (t, J=7.2 Hz, 1H), 6.42 (d, J=7.6 Hz, 2H), 5.49 (s, 2H), 3.87 (t, J=7.6 Hz, 2H), 3.54-3.60 (m, 4H), 3.21 (s, 3H), 2.89-2.95 (m, 1H). LC-MS m/z: 517.3 [M+H]⁺. HPLC Purity (254 nm): 95.53%; t_{R}=9.172 min.

AC2020601-0472

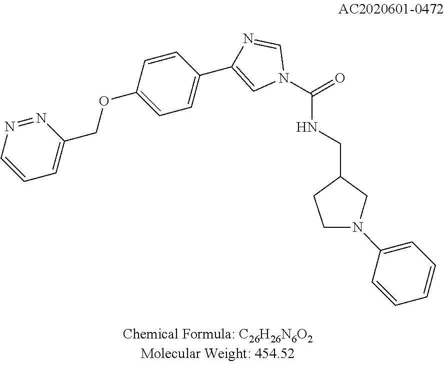 [SMILES: O=C(NCC1CCN(c2ccccc2)C1)n1cnc(-c2ccc(OCc3cccnn3)cc2)c1]

### The Synthesis of tert-butyl 4-(4-(pyridazin-3-ylmethoxy)phenyl)-1H-imidazole-1-carboxylate (472-2)

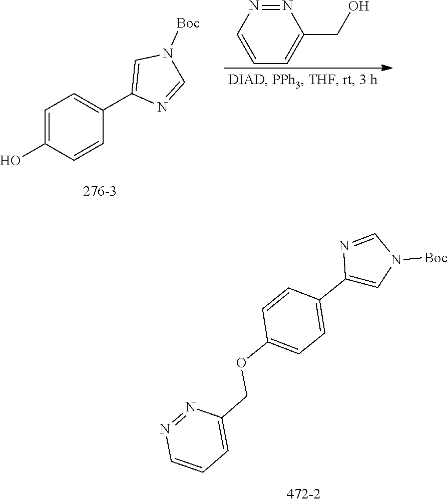 [SMILES: CC(C)(C)OC(=O)n1cnc(-c2ccc(O)cc2)c1 ; OCc1cccnn1 ; CC(C)(C)OC(=O)n1cnc(-c2ccc(OCc3cccnn3)cc2)c1]

To a suspension of 276-3 (780 mg, 3.0 mmol) in THF (10 mL) was added pyridazin-3-ylmethanol (660 mg, 6.0 mmol), PPh₃ (943 mg, 3.6 mmol) and DIAD (787 mg, 3.9 mmol). The reaction mixture was stirred at room temperature for 3 hours until the reaction was complete (by LCMS). Water was added and the reaction mixture was extracted with EtOAc (50 mL), the combined organic layer was washed with brine, dried over anhydrous Na₂SO₄ and evaporated under reduced pressure. The crude was purified by silica gel chromatography (30% EA/PE) to afford 472-2 (500 mg, yield: 47.3%) as white oil.

### The Synthesis of 3-((4-(1H-imidazol-4-yl)phenoxy)methyl)pyridazine (472-3)

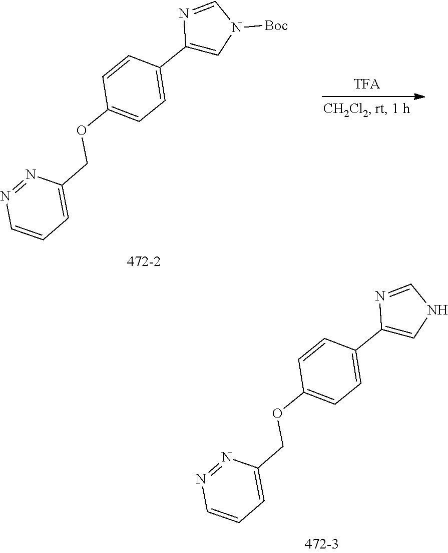 [SMILES: CC(C)(C)OC(=O)n1cnc(-c2ccc(OCc3cccnn3)cc2)c1 ; c1cnnc(COc2ccc(-c3c[nH]cn3)cc2)c1]

To a stirred solution of 472-2 (500 mg, 1.42 mmol) in CH₂Cl₂ (10 mL) was added TFA (10 mL). The reaction mixture was stirred at room temperature for 1 hour until the reaction was complete (by LCMS). The reaction mixture was quenched with water, extracted with DCM (20 mL), the combined organic layer was washed with brine, dried over anhydrous Na₂SO₄. The mixture was concentrated under reduced pressure to afford 472-3 (300 mg, yield: 92.5%) as yellow oil, which was used to the next step without purification.

### The Synthesis of N-((1-phenylpyrrolidin-3-yl)methyl)-4-(4-(pyridazin-3-ylmethoxy)phenyl)-1H-imidazole-1-carboxamide (AC2020601-0472)

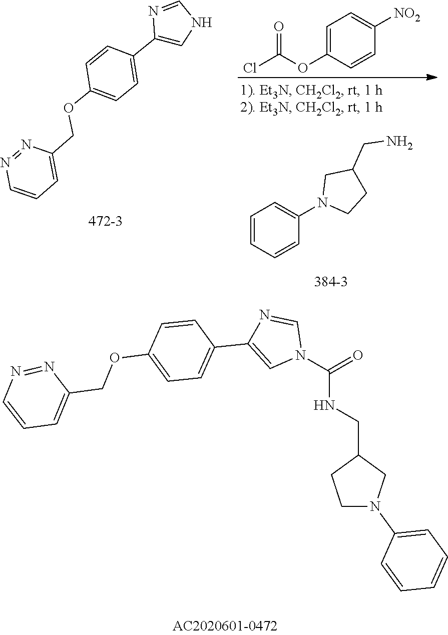 [SMILES: c1cnnc(COc2ccc(-c3c[nH]cn3)cc2)c1 ; NCC1CCN(c2ccccc2)C1 ; O=C(Cl)Oc1ccc([N+](=O)[O-])cc1 ; O=C(NCC1CCN(c2ccccc2)C1)n1cnc(-c2ccc(OCc3cccnn3)cc2)c1]

To a solution of 472-3 (300 mg, 1.19 mmol) in DCM (5 mL) was added 4-nitrophenyl carbonochloridate (358 mg, 1.78 mmol) and Et₃N (5 mL) at room temperature and stirred for 1 hour. Then (1-phenylpyrrolidin-3-yl)methanamine (384-3, 419 mg, 2.38 mmol) was added. After 1 hour stirring, the reaction mixture was quenched with water, extracted with DCM (20 mL), the combined organic layer was washed with brine, dried over anhydrous Na₂SO₄ and evaporated under reduced pressure. The crude was purified by silica gel chromatography (5% MeOH/DCM) to afford AC2020601-0472 (12 mg, yield: 2.1%) as a white solid. ¹H NMR (400 MHz, DMSO-d₆) δ 8.22 (dd, J=4.8 Hz, J=1.2 Hz, 1H), 8.67 (t, J=5.2 Hz, 1H), 8.30 (d, J=0.8 Hz, 1H), 8.05 (s, 1H), 7.84 (dd, J=8.4 Hz, J=1.6 Hz, 1H), 7.74-7.78 (m, 3H), 7.15 (t, J=7.6 Hz, 2H), 7.10 (d, J=8.8 Hz, 2H), 6.58 (t, J=7.2 Hz, 1H), 6.53 (d, J=8.0 Hz, 2H), 5.44 (s, 2H), 3.36-3.40 (m, 4H), 3.21-3.27 (m, 1H), 3.05-3.09 (m, 1H), 2.59-2.67 (m, 1H), 2.10-2.15 (m, 1H), 1.78-1.83 (m, 1H). LC-MS m/z: 455.3 [M+H]⁺. HPLC Purity (254 nm): 92.90%; t_{R}=8.756 min.

AC2020601-0477

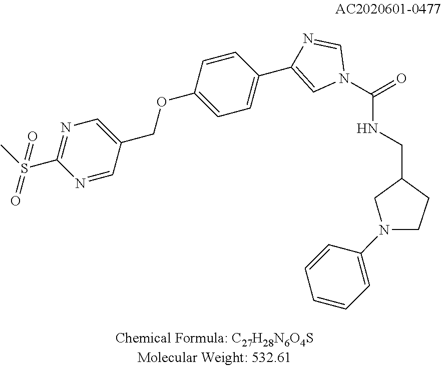 [SMILES: CS(=O)(=O)c1ncc(COc2ccc(-c3cn(C(=O)NCC4CCN(c5ccccc5)C4)cn3)cc2)cn1]

### The Synthesis of (2-(methylthio)pyrimidin-5-yl)methanol (477-2)

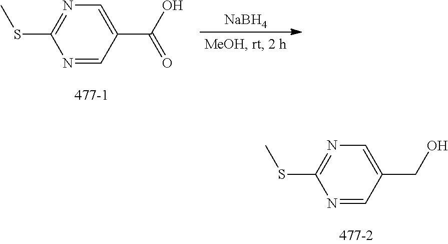

To a suspension of 477-1 (2.0 g, 11.8 mmol) in MeOH (20 mL) was added NaBH₄ (2.2 g, 59.0 mmol). The reaction mixture was stirred at room temperature for 2 hours until the reaction was complete (by LCMS). Water was added and the reaction mixture was extracted with EtOAc (50 mL), the combined organic layer was washed with brine, dried over anhydrous Na₂SO₄ and evaporated under reduced pressure. The crude was purified by silica gel chromatography (30% EA/PE) to afford 477-2 (1.0 g, yield: 54%) as white oil.

### The Synthesis of tert-butyl 4-(4-((2-(methylthio)pyrimidin-5-yl)methoxy)phenyl)-1H-imidazole-1-carboxylate (477-3)

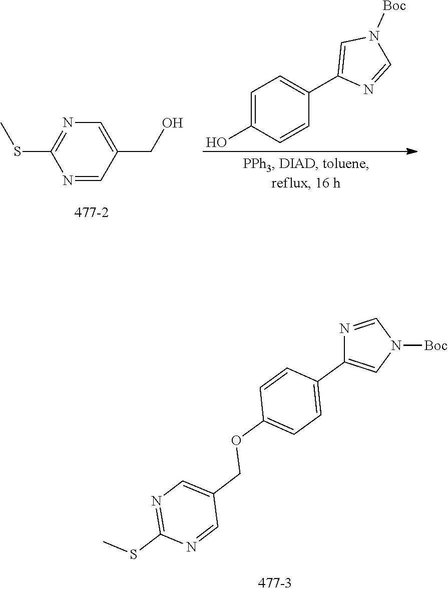

To a suspension of 477-2 (1.0 g, 6.4 mmol) in Tol (10 mL) was added tert-butyl 4-(4-hydroxyphenyl)-1H-imidazole-1-carboxylate (832 mg, 3.2 mmol), PPh₃ (1.0 g, 3.8 mmol) and DIAD (840 mg, 4.2 mmol). The reaction mixture was allowed to warm to 110° C. for 16 hours until the reaction was complete (by LCMS). Water was added and the reaction mixture was extracted with EtOAc (50 mL), the combined organic layer was washed with brine, dried over anhydrous Na₂SO₄ and evaporated under reduced pressure. The crude was purified by silica gel chromatography (30% EA/PE) to afford 477-3 (800 mg, yield: 31%) as yellow oil.

### The Synthesis of tert-butyl 4-(4-((2-(methylsulfonyl)pyrimidin-5-yl)methoxy)phenyl)-1H-imidazole-1-carboxylate (477-4)

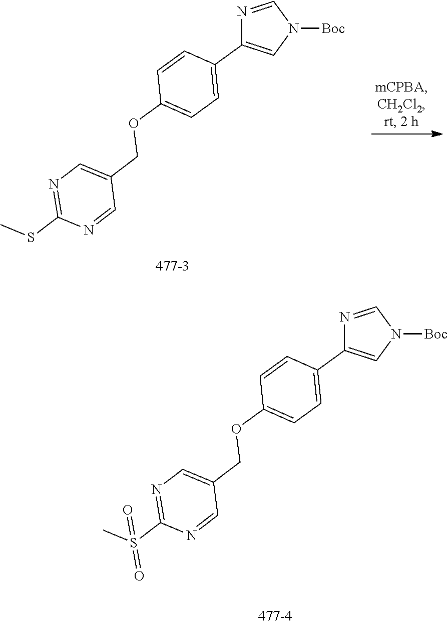 [SMILES: CSc1ncc(COc2ccc(-c3cn(C(=O)OC(C)(C)C)cn3)cc2)cn1 ; CC(C)(C)OC(=O)n1cnc(-c2ccc(OCc3cnc(S(C)(=O)=O)nc3)cc2)c1]

To a suspension of 477-3 (800 mg, 2.0 mmol) in DCM (10 mL) was added mCPBA (520 mg, 3.0 mmol). The reaction mixture was stirred at room temperature for 2 hours until the reaction was complete (by LCMS). Water was added and the reaction mixture was extracted with DCM (50 mL), the combined organic layer was washed with brine, dried over anhydrous Na₂SO₄ and evaporated under reduced pressure. The crude was purified by silica gel chromatography (40% EA/PE) to afford 477-4 (760 mg, yield: 88%) as white oil.

### The Synthesis of 5-((4-(1H-imidazol-4-yl)phenoxy)methyl)-2-(methylsulfonyl)pyrimidine (477-5)

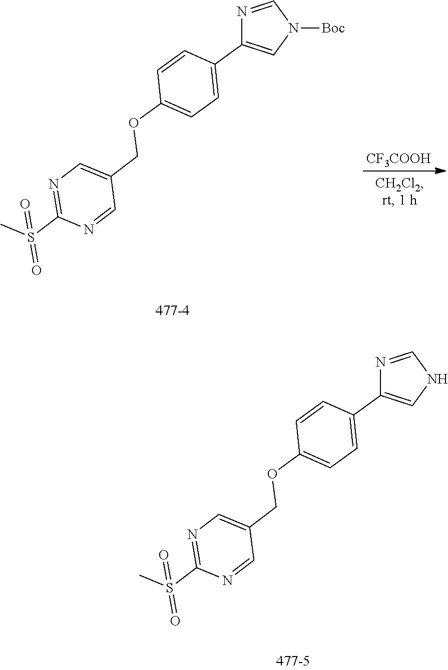 [SMILES: CC(C)(C)OC(=O)n1cnc(-c2ccc(OCc3cnc(S(C)(=O)=O)nc3)cc2)c1 ; "CF3COOH" ; CS(=O)(=O)c1ncc(COc2ccc(-c3c[nH]cn3)cc2)cn1]

To a stirred solution of 477-4 (760 mg, 1.78 mmol) in CH₂Cl₂ (10 mL) was added TFA (10 mL). The reaction mixture was stirred at room temperature for 1 hour until the reaction was complete (by LCMS). The reaction mixture was quenched with water, extracted with DCM (20 mL), the combined organic layer was washed with brine, dried over anhydrous Na₂SO₄. The mixture was concentrated under reduced pressure to afford 477-5 (500 mg, yield: 85%) as yellow oil, which was used to the next step without purification.

### The Synthesis of 4-(4-((2-(methylsulfonyl)pyrimidin-5-yl)methoxy)phenyl)-N-((1-phenylpyrrolidin-3-yl)methyl)-1H-imidazole-1-carboxamide (AC2020601-0477)

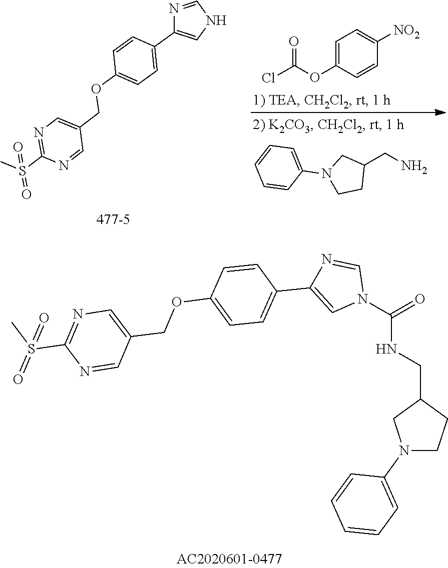 [SMILES: CS(=O)(=O)c1ncc(COc2ccc(-c3c[nH]cn3)cc2)cn1 ; NCC1CCN(c2ccccc2)C1 ; O=C(Cl)Oc1ccc([N+](=O)[O-])cc1 ; CS(=O)(=O)c1ncc(COc2ccc(-c3cn(C(=O)NCC4CCN(c5ccccc5)C4)cn3)cc2)cn1]

To a solution of 477-5 (500 mg, 1.5 mmol) in DCM (5 mL) was added 4-nitrophenyl carbonochloridate (450 mg, 2.25 mmol) and Et₃N (5 mL) at room temperature and stirred for 1 hour. Then (1-phenylpyrrolidin-3-yl)methanamine (530 mg, 3.0 mmol) and K₂CO₃ (414 mg, 3.0 mmol) was added. After 1 hour stirring, the reaction mixture was quenched with water, extracted with DCM (20 mL), the combined organic layer was washed with brine, dried over anhydrous Na₂SO₄ and evaporated under reduced pressure. The crude was purified by silica gel chromatography (5% MeOH/DCM) to afford AC2020601-0477 (87 mg, yield: 10.8%) as an off-white solid. ¹H NMR (400 MHz, DMSO-d₆) δ 9.21 (s, 2H), 9.15 (s, 1H), 8.41 (d, J=0.8 Hz, 1H), 8.30 (s, 1H), 7.77 (d, J=8.8 Hz, 2H), 7.11-7.17 (m, 4H), 6.58 (t, J=7.2 Hz, 1H), 6.53 (d, J=8.0 Hz, 2H), 5.36 (s, 2H), 3.44 (s, 3H), 3.31-3.34 (m, 4H), 3.22-3.26 (m, 1H), 3.06-3.10 (m, 1H), 2.65-2.69 (m, 1H), 2.10-2.12 (m, 1H), 1.82-1.85 (m, 1H). LC-MS m/z: 533.2 [M+H]⁺. HPLC Purity (254 nm): 98.50%; t_{R}=1.690 min.

AC2020601-0478

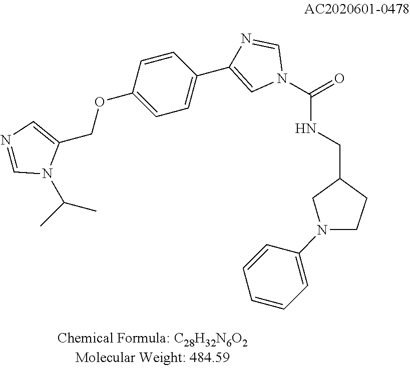 [SMILES: CC(C)n1cncc1COc1ccc(-c2cn(C(=O)NCC3CCN(c4ccccc4)C3)cn2)cc1]

### The Synthesis of methyl 1-isopropyl-1H-imidazole-5-carboxylate (478-1-2)

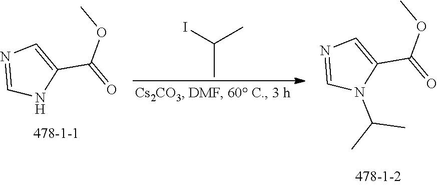

To a suspension of 478-1-1 (3.75 g, 30.0 mmol) in DMF (15 mL) was added 2-iodopropane (15.0 g, 90.0 mmol) and Cs₂CO₃ (20.0 g, 60.0 mmol). The reaction mixture was allowed to warm to 60° C. for 3 hours until the reaction was complete (by LCMS). Water was added and the reaction mixture was extracted with EtOAc (50 mL), the combined organic layer was washed with brine, dried over anhydrous Na₂SO₄ and evaporated under reduced pressure. The crude was purified by silica gel chromatography (15% PE/EA) to afford 478-1-2 (2.5 g, yield: 50.1%) as white oil.

### The Synthesis of (1-isopropyl-1H-imidazol-5-yl)methanol (478-1-3)

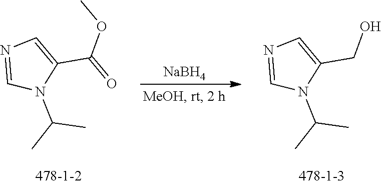

To a suspension of 478-1-2 (2.5 g, 14.9 mmol) in MeOH (20 mL) was added NaBH₄ (2.8 g, 74.5 mmol). The reaction mixture was stirred at room temperature for 2 hours until the reaction was complete (by LCMS). Water was added and the reaction mixture was extracted with EtOAc (50 mL), the combined organic layer was washed with brine, dried over anhydrous Na₂SO₄ and evaporated under reduced pressure. The crude was purified by silica gel chromatography (15% PE/EA) to afford 478-1-3 (800 mg, yield: 45.2%) as white oil.

### The Synthesis of tert-butyl 4-(4-((1-isopropyl-1H-imidazol-5-yl)methoxy)phenyl)-1H-imidazole-1-carboxylate (478-2)

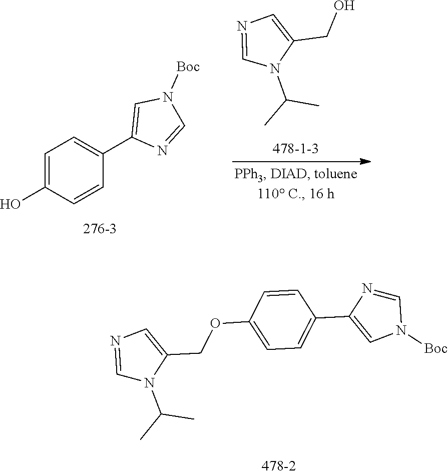

To a suspension of 478-1-3 (800 mg, 5.7 mmol) in THF (20 mL) was added 276-3 (741 mg, 2.85 mmol), PPh₃ (900 mg, 3.42 mmol) and DIAD (750 mg, 3.7 mmol). The reaction mixture was allowed to warm to 100° C. for 16 hours until the reaction was complete (by LCMS). Water was added and the reaction mixture was extracted with EtOAc (50 mL), the combined organic layer was washed with brine, dried over anhydrous Na₂SO₄ and evaporated under reduced pressure. The crude was purified by silica gel chromatography (45% EA/PE) to afford 478-2 (500 mg, yield: 22.4%) as yellow oil.

### The Synthesis of 5-((4-(1H-imidazol-4-yl)phenoxy)methyl)-1-isopropyl-1H-imidazole (478-3)

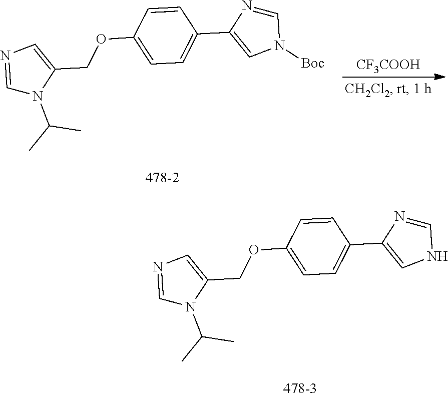 [SMILES: CC(C)n1cncc1COc1ccc(-c2cn(C(=O)OC(C)(C)C)cn2)cc1 ; "CF3COOH" ; CC(C)n1cncc1COc1ccc(-c2c[nH]cn2)cc1]

To a stirred solution of 478-2 (500 mg, 1.3 mmol) in CH₂Cl₂ (10 mL) was added TFA (10 mL). The reaction mixture was stirred at room temperature for 1 hour until the reaction was complete (by LCMS). The reaction mixture was quenched with water, extracted with DCM (20 mL), the combined organic layer was washed with brine, dried over anhydrous Na₂SO₄. The mixture was concentrated under reduced pressure to afford 478-3 (150 mg, yield: 41.1%) as yellow oil, which was used to the next step without purification.

### The Synthesis of 4-(4-((1-isopropyl-1H-imidazol-5-yl)methoxy)phenyl)-N-((1-phenylpyrrolidin-3-yl)methyl)-1H-imidazole-1-carboxamide (AC2020601-0478)

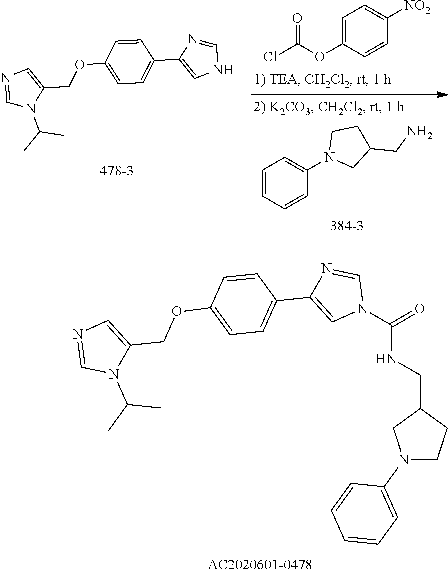 [SMILES: CC(C)n1cncc1COc1ccc(-c2c[nH]cn2)cc1 ; NCC1CCN(c2ccccc2)C1 ; O=C(Cl)Oc1ccc([N+](=O)[O-])cc1 ; CC(C)n1cncc1COc1ccc(-c2cn(C(=O)NCC3CCN(c4ccccc4)C3)cn2)cc1]

To a solution of 478-3 (150 mg, 0.53 mmol) in DCM (5 mL) was added 4-nitrophenyl carbonochloridate (161 mg, 0.8 mmol) and Et₃N (5 mL) at room temperature and stirred for 1 hour. Then (1-phenylpyrrolidin-3-yl)methanamine (384-3, 187 mg, 1.06 mmol) and K₂CO₃ (146 mg, 1.06 mmol) was added. After 1 hour stirring, the reaction mixture was quenched with water, extracted with DCM (20 mL), the combined organic layer was washed with brine, dried over anhydrous Na₂SO₄ and evaporated under reduced pressure. The crude was purified by silica gel chromatography (5% MeOH/DCM) to afford AC2020601-0478 (5.89 mg, yield: 1.8%) as yellow solid. ¹H NMR (400 MHz, DMSO-d₆) δ 8.68 (s, 1H), 8.30 (d, J=0.8 Hz, 1H), 8.04 (d, J=0.8 Hz, 1H), 7.86 (s, 1H), 7.74 (d, J=8.8 Hz, 2H), 7.15 (t, J=7.6 Hz, 2H), 7.07 (d, J=8.8 Hz, 2H), 7.02 (s, 1H), 6.58 (t, J=7.6 Hz, 1H), 6.53 (d, J=8.0 Hz, 2H), 5.12 (s, 2H), 4.37-4.43 (m, 1H), 3.34-3.40 (m, 4H), 3.21-3.27 (m, 1H), 3.04-3.09 (m, 1H), 2.59-2.65 (m, 1H), 2.10-2.15 (m, 1H), 1.78-1.83 (m, 1H), 1.44 (d, J=6.8 Hz, 6H). LC-MS m/z: 485.3 [M+H]⁺. HPLC Purity (254 nm): 91.02%; t_{R}=3.657 min.

AC2020601-0479

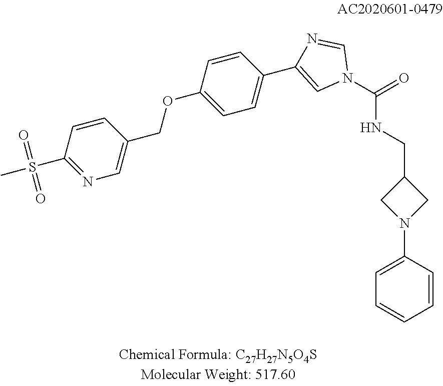 [SMILES: CS(=O)(=O)c1ccc(COc2ccc(-c3cn(C(=O)NCC4CN(c5ccccc5)C4)cn3)cc2)cn1]

### The Synthesis of 4-(4-((6-(methylsulfonyl)pyridin-3-yl)methoxy)phenyl)-N-((1-phenylazetidin-3-yl)methyl)-1H-imidazole-1-carboxamide (AC2020601-0479)

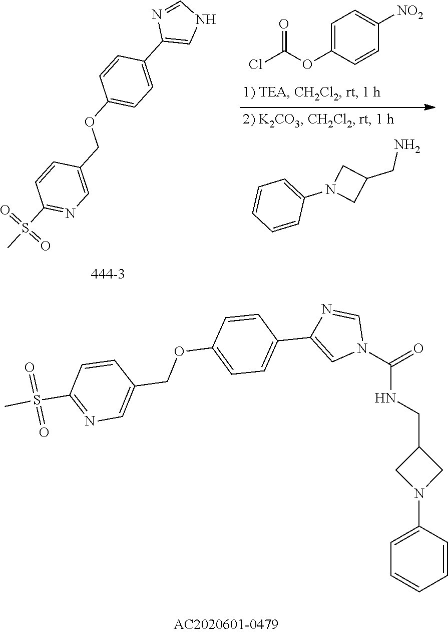 [SMILES: CS(=O)(=O)c1ccc(COc2ccc(-c3c[nH]cn3)cc2)cn1 ; NCC1CN(c2ccccc2)C1 ; O=C(Cl)Oc1ccc([N+](=O)[O-])cc1 ; CS(=O)(=O)c1ccc(COc2ccc(-c3cn(C(=O)NCC4CN(c5ccccc5)C4)cn3)cc2)cn1]

To a solution of 444-3 (130 mg, 0.4 mmol) in DCM (5 mL) was added 4-nitrophenyl carbonochloridate (120 mg, 0.6 mmol) and Et₃N (5 mL) at room temperature and stirred for 1 hour. Then (1-phenylazetidin-3-yl)methanamine (130 mg, 0.8 mmol) and K₂CO₃ (110 mg, 0.8 mmol)was added. After 1 hour stirring, the reaction mixture was quenched with water, extracted with DCM (20 mL), the combined organic layer was washed with brine, dried over anhydrous Na₂SO₄ and evaporated under reduced pressure. The crude was purified by silica gel chromatography (5% MeOH/DCM) to afford AC2020601-0479 (23 mg, yield: 8.1%) as a white solid. ¹H NMR (400 MHz, DMSO-d₆) δ 8.69 (t, J=5.2 Hz, 1H), 8.51 (d, J=2.0 Hz, 1H), 8.28 (d, J=0.8 Hz, 1H), 8.02 (s, 1H), 7.85 (dd, J=8.0 Hz, J=2.4 Hz, 1H), 7.69-7.75 (m, 3H), 7.16 (t, J=7.6 Hz, 2H), 7.06 (d, J=8.8 Hz, 2H), 6.66 (t, J=7.2 Hz, 1H), 6.42 (d, J=7.6 Hz, 2H), 5.17 (s, 2H), 3.88 (t, J=7.2 Hz, 2H), 3.55-3.60 (m, 4H), 3.35 (s, 3H), 2.91-2.96 (m, 1H). LC-MS m/z: 518.3 [M+H]⁺. HPLC Purity (254 nm): 94.23%; t_{R}=3.851 min.

AC2020601-0480

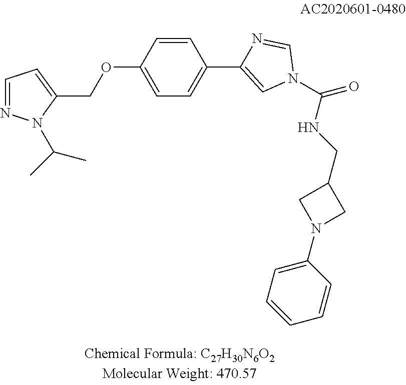 [SMILES: CC(C)n1nccc1COc1ccc(-c2cn(C(=O)NCC3CN(c4ccccc4)C3)cn2)cc1]

### The Synthesis of methyl 1-isopropyl-1H-pyrazole-5-carboxylate (448A-1-2)

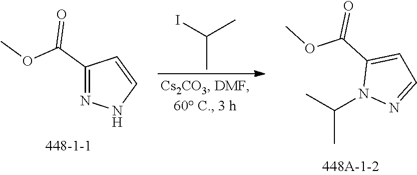

To a suspension of 448-1-1 (2.5 g, 20.0 mmol) in DMF (15 mL) was added 2-iodopropane (10.0 g, 60.0 mmol) and Cs₂CO₃ (13.0 g, 40.0 mmol). The reaction mixture was allowed to warm to 60° C. for 3 hours until the reaction was complete (by LCMS). Water was added and the reaction mixture was extracted with EtOAc (50 mL), the combined organic layer was washed with brine, dried over anhydrous Na₂SO₄ and evaporated under reduced pressure. The crude was purified by silica gel chromatography (15% PE/EA) to afford 448A-1-2 (1.2 g, yield: 37.1%) as white oil.

### The Synthesis of (1-isopropyl-1H-pyrazol-5-yl)methanol (448A-1-3)

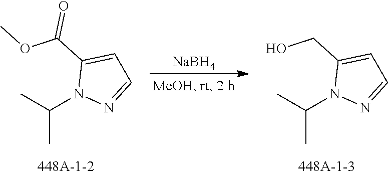

To a suspension of 448A-1-2 (1.2 g, 7.1 mmol) in MeOH (20 mL) was added NaBH₄ (1.3 g, 35.5 mmol). The reaction mixture was stirred at room temperature for 2 hours until the reaction was complete (by LCMS). Water was added and the reaction mixture was extracted with EtOAc (50 mL), the combined organic layer was washed with brine, dried over anhydrous Na₂SO₄ and evaporated under reduced pressure. The crude was purified by silica gel chromatography (15% PE/EA) to afford 448A-1-3 (800 mg, yield: 77.2%) as white oil.

### The Synthesis of tert-butyl 4-(4-((1-isopropyl-1H-pyrazol-5-yl)methoxy)phenyl)-1H-imidazole-1-carboxylate (448A-2)

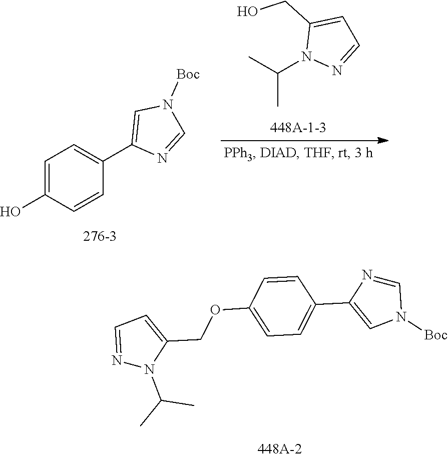

To a suspension of 448A-1-3 (800 mg, 4.75 mmol) in THF (20 mL) was added 276-3 (618 mg, 2.38 mmol), PPh₃ (746 mg, 2.85 mmol) and DIAD (626 mg, 3.1 mmol). The reaction mixture was stirred at room temperature for 3 hours until the reaction was complete (by LCMS). Water was added and the reaction mixture was extracted with EtOAc (50 mL), the combined organic layer was washed with brine, dried over anhydrous Na₂SO₄ and evaporated under reduced pressure. The crude was purified by silica gel chromatography (45% EA/PE) to afford 448A-2 (500 mg, yield: 22.4%) as yellow oil.

### The Synthesis of 5-((4-(1H-imidazol-4-yl)phenoxy)methyl)-1-isopropyl-1H-pyrazole (448A-3)

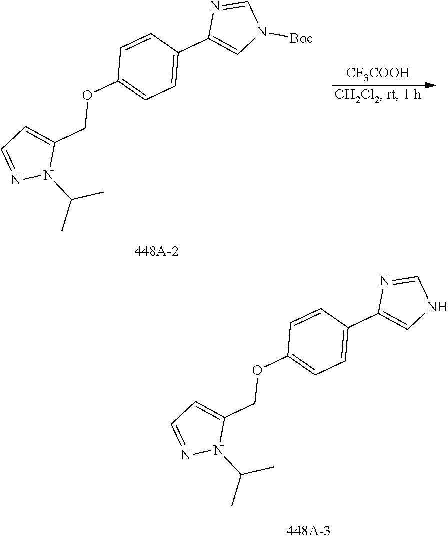 [SMILES: CC(C)n1nccc1COc1ccc(-c2cn(C(=O)OC(C)(C)C)cn2)cc1 ; "CF3COOH" ; "CH2Cl2, rt, 1 h" ; CC(C)n1nccc1COc1ccc(-c2c[nH]cn2)cc1]

To a stirred solution of 448A-2 (500 mg, 1.3 mmol) in CH₂Cl₂ (10 mL) was added TFA (10 mL). The reaction mixture was stirred at room temperature for 1 hour until the reaction was complete (by LCMS). The reaction mixture was quenched with water, extracted with DCM (20 mL), the combined organic layer was washed with brine, dried over anhydrous Na₂SO₄. The mixture was concentrated under reduced pressure to afford 448A-3 (250 mg, yield: 67.6%) as yellow oil, which was used to the next step without purification.

### The Synthesis of 4-(4-((1-isopropyl-1H-pyrazol-3-yl)methoxy)phenyl)-N-((1-phenylpyrrolidin-3-yl)methyl)-1H-imidazole-1-carboxamide (AC2020601-0480)

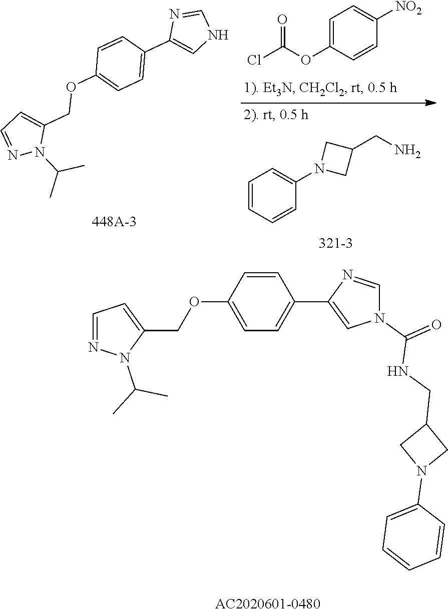 [SMILES: CC(C)n1nccc1COc1ccc(-c2c[nH]cn2)cc1 ; NCC1CN(c2ccccc2)C1 ; O=C(Cl)Oc1ccc([N+](=O)[O-])cc1 ; CC(C)n1nccc1COc1ccc(-c2cn(C(=O)NCC3CN(c4ccccc4)C3)cn2)cc1]

To a solution of 448A-3 (250 mg, 0.89 mmol) in DCM (5 mL) was added 4-nitrophenyl carbonochloridate (269 mg, 1.34 mmol) and Et₃N (5 mL) at room temperature and stirred for 0.5 hour. Then (1-phenylazetidin-3-yl)methanamine (321-3, 288 mg, 1.78 mmol) was added. After 0.5 hour stirring, the reaction mixture was quenched with water, extracted with DCM (20 mL), the combined organic layer was washed with brine, dried over anhydrous Na₂SO₄ and evaporated under reduced pressure. The crude was purified by silica gel chromatography (5% MeOH/DCM) to afford AC2020601-0480 (7 mg, yield: 1.2%) as a white solid. ¹H NMR (400 MHz, DMSO-d₆) δ 8.69 (s, 1H), 8.28 (d, J=1.2 Hz, 1H), 8.02 (d, J=1.2 Hz, 1H), 7.74 (d, J=8.8 Hz, 2H), 7.43 (d, J=1.6 Hz, 1H), 7.16 (dd, J=8.4 Hz, J=7.6 Hz, 2H), 7.01 (d, J=8.4 Hz, 2H), 6.66 (t, J=7.2 Hz, 1H), 6.42 (d, J=7.6 Hz, 2H), 6.36 (d, J=1.6 Hz, 1H), 5.20 (s, 2H), 4.58-4.64 (m, 1H), 3.88 (t, J=7.6 Hz, 2H), 3.55-3.60 (m, 4H), 2.90-2.96 (m, 1H), 2.49-2.51 (m, 6H). LC-MS m/z: 471.3 [M+H]⁺. HPLC Purity (254 nm): 93.57%; t_{R}=9.043 min.

AC2020601-0481

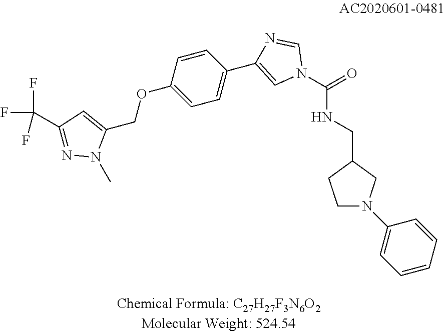 [SMILES: Cn1nc(C(F)(F)F)cc1COc1ccc(-c2cn(C(=O)NCC3CCN(c4ccccc4)C3)cn2)cc1]

### The Synthesis of tert-butyl 4-(4-((1-methyl-3-(trifluoromethyl)-1H-pyrazol-5-yl)methoxy)phenyl)-1H-imidazole-1-carboxylate (481-2)

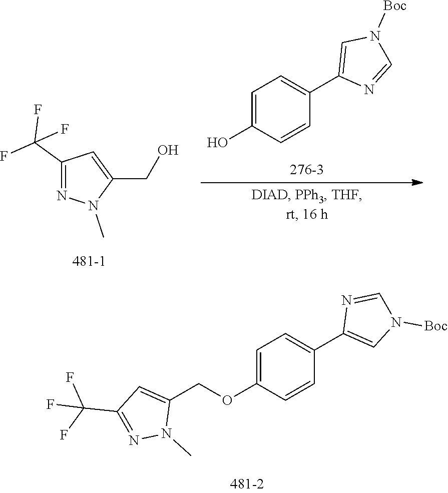 [SMILES: Cn1nc(C(F)(F)F)cc1CO ; CC(C)(C)OC(=O)n1cnc(-c2ccc(O)cc2)c1 ; Cn1nc(C(F)(F)F)cc1COc1ccc(-c2cn(C(=O)OC(C)(C)C)cn2)cc1]

To a suspension of 276-3 (780 mg, 3.0 mmol) in THF (10 mL) was added (1-methyl-3-(trifluoromethyl)-1H-pyrazol-5-yl)methanol (540 mg, 3.0 mmol), PPh₃ (942.0 mg, 3.6 mmol) and DIAD (789.0 mg, 3.9 mmol). The reaction mixture was stirred at room temperature for 3 hours until the reaction was complete (by LCMS). Saturated NaCl solution was added and the reaction mixture was extracted with EtOAc (20 mL), the combined organic layer was washed with brine, dried over anhydrous Na₂SO₄ and evaporated under reduced pressure. The crude was purified by silica gel chromatography (10% MeOH/DCM) to afford 481-2 (665 mg, yield: 52%) as yellow oil.

### The Synthesis of 5-((4-(1H-imidazol-4-yl)phenoxy)methyl)-1-methyl-3-(trifluoromethyl)-1H-pyrazole (481-3)

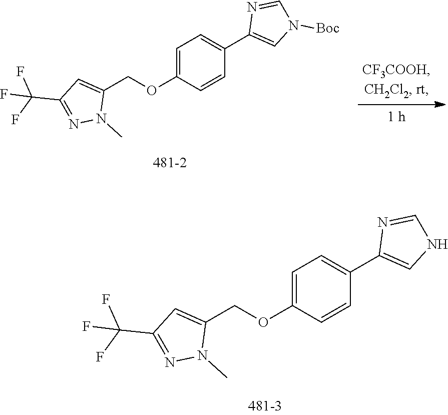 [SMILES: Cn1nc(C(F)(F)F)cc1COc1ccc(-c2cn(C(=O)OC(C)(C)C)cn2)cc1 ; Cn1nc(C(F)(F)F)cc1COc1ccc(-c2c[nH]cn2)cc1]

To a stirred solution of 481-2 (422 mg, 1.00 mmol) in CH₂Cl₂ (10 mL) was added TFA (5 mL). The reaction mixture was stirred at room temperature for 1 hour until the reaction was complete (by LCMS). The mixture was concentrated under reduced pressure to afford 481-3 (320 mg, crude) as yellow oil, which was used to the next step without purification.

### The Synthesis of 4-(4-((1-methyl-3-(trifluoromethyl)-1H-pyrazol-5-yl)methoxy)phenyl)-N-((1-phenylpyrrolidin-3-yl)methyl)-1H-imidazole-1-carboxamide (AC2020601-0481)

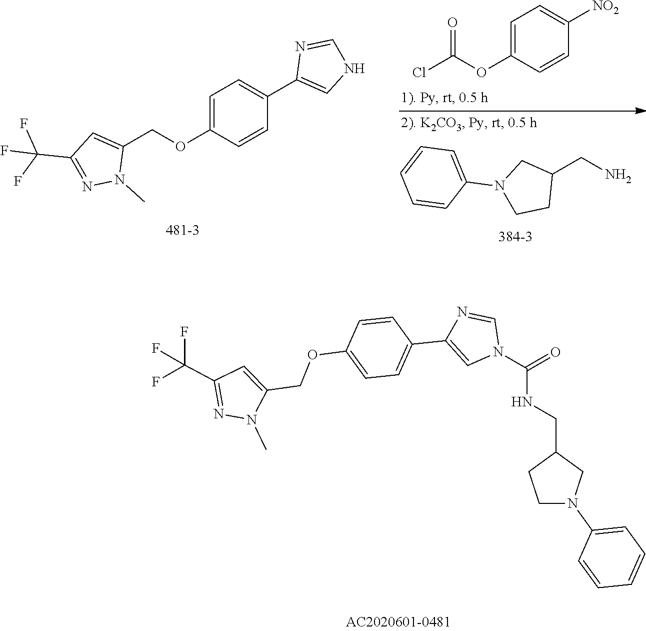 [SMILES: Cn1nc(C(F)(F)F)cc1COc1ccc(-c2c[nH]cn2)cc1 ; NCC1CCN(c2ccccc2)C1 ; O=C(Cl)Oc1ccc([N+](=O)[O-])cc1 ; Cn1nc(C(F)(F)F)cc1COc1ccc(-c2cn(C(=O)NCC3CCN(c4ccccc4)C3)cn2)cc1]

To a solution of 481-3 (129 mg, 0.4 mmol) in Py (5 mL) was added SM1 (82.0 mg, 0.4 mmol) at 0° C. and stirred for 0.5 hour. Then the reaction mixture was allowed to warm to room temperature and K₂CO₃ (138 mg, 1.0 mmol), 384-3 (71.0 mg, 0.4 mmol) was added. After 0.5 hour stirring, the reaction mixture was quenched with water, extracted with DCM (20 mL), the combined organic layer was washed with brine, dried over anhydrous Na₂SO₄ and evaporated under reduced pressure. The crude was purified by silica gel chromatography (10% MeOH/DCM) to afford AC2020601-0481 (57 mg, yield: 27.2%) as a white solid. ¹H NMR (400 MHz, DMSO-d₆) δ 8.67 (t, J=5.6 Hz, 1H), 8.31 (d, J=1.2 Hz, 1H), 8.06 (d, J=1.2 Hz, 1H), 7.76 (d, J=8.8 Hz, 2H), 7.10-7.18 (m, 4H), 6.89 (s, 1H), 6.52-6.60 (m, 3H), 5.26 (s, 2H), 3.95 (s, 3H), 3.30-3.41 (m, 4H), 3.21-3.28 (m, 1H), 3.05-3.09 (m, 1H), 2.61-2.65 (m, 1H), 2.11-2.17 (m, 1H), 1.78-1.85 (m, 1H). LC-MS m/z: 525.2 [M+H]⁺. HPLC Purity (254 nm): 100%; t_{R}=4.225 min.

AC2020601-0482

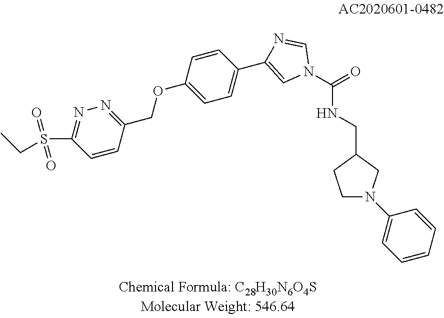 [SMILES: CCS(=O)(=O)c1ccc(COc2ccc(-c3cn(C(=O)NCC4CCN(c5ccccc5)C4)cn3)cc2)nn1]

### The Synthesis of methyl 6-(ethylthio)pyridazine-3-carboxylate (482-2)

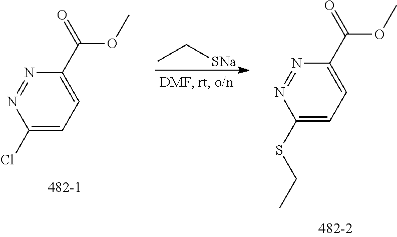

To a suspension of 482-1 (8 g, 46.36 mmol) in DMF (50 mL) was added sodium ethanethiolate (7.80 g, 92.72 mmol). The reaction mixture was stirred at room temperature for 16 hours until the reaction was complete (by LCMS). Saturated NaCl solution was added and the reaction mixture was extracted with EtOAc (20 mL), the combined organic layer was washed with brine, dried over anhydrous Na₂SO₄ and evaporated under reduced pressure. The crude was purified by silica gel chromatography (10% MeOH/DCM) to afford 482-2 (8 g, yield: 87.01%) as a yellow solid.

### The Synthesis of (6-(ethylthio)pyridazin-3-yl)methanol (482-3)

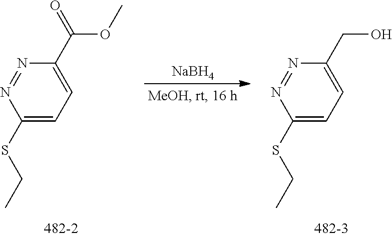

To a solution of 482-2 (8 g, 40.35 mmol) in MeOH (100 mL) was added NaBH₄ (3.05 g, 80.71 mmol). The reaction mixture was stirred at room temperature for 16 hours until the reaction was complete (by LCMS). Saturated NH₄Cl solution was added and the reaction mixture was extracted with EA (100 mL), the combined organic layer was washed with brine, dried over anhydrous Na₂SO₄ and evaporated under reduced pressure. The crude was purified by silica gel chromatography (10% MeOH/DCM) to afford 482-3 (3 g, yield: 44%) as yellow oil.

### The Synthesis of tert -butyl 4-(4-((6-(ethylthio)pyridazin-3-yl)methoxy)phenyl)-1H-imidazole-1-carboxylate (482-4)

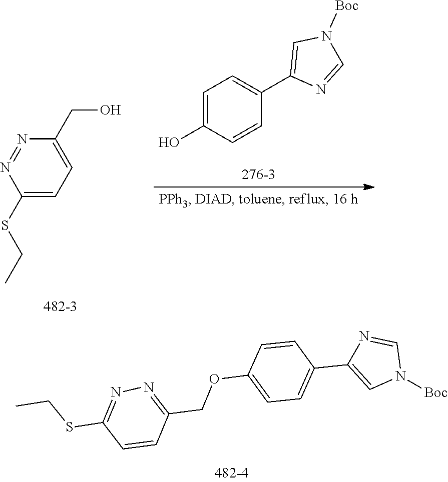 [SMILES: CCSc1ccc(CO)nn1 ; CC(C)(C)OC(=O)n1cnc(-c2ccc(O)cc2)c1 ; CCSc1ccc(COc2ccc(-c3cn(C(=O)OC(C)(C)C)cn3)cc2)nn1]

To a suspension of 276-3 (780 mg, 3.0 mmol) in Tol (20 mL) was added 482-3 (510 mg, 3.0 mmol), PPh₃ (942.0 mg, 3.6 mmol) and DIAD (789.0 mg, 3.9 mmol). The reaction mixture was stirred 110° C. for 3 hours until the reaction was complete (by LCMS). Saturated NaCl solution was added and the reaction mixture was extracted with EtOAc (50 mL), the combined organic layer was washed with brine, dried over anhydrous Na₂SO₄ and evaporated under reduced pressure. The crude was purified by silica gel chromatography (10% MeOH/DCM) to afford 482-4 (700 mg, yield: 57%) as yellow oil.

### The Synthesis of tert-butyl 4-(4-((6-(ethylsulfonyl)pyridazin-3-yl)methoxy)phenyl)-1H-imidazole-1-carboxylate (482-5)

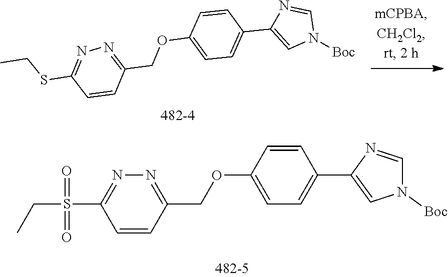 [SMILES: CCSc1ccc(COc2ccc(-c3cn(C(=O)OC(C)(C)C)cn3)cc2)nn1 ; CCS(=O)(=O)c1ccc(COc2ccc(-c3cn(C(=O)OC(C)(C)C)cn3)cc2)nn1]

To a solution of 482-4 (0.70 g, 1.70 mmol) in CH₂Cl₂ (10 mL) was added mCPBA (586 mg, 3.39 mmol). The reaction mixture was stirred at room temperature for 2 hours until the reaction was complete (by LCMS). Saturated Na₂SO₃ solution was added and the reaction mixture was extracted with CH₂Cl₂ (20 mL), the combined organic layer was washed with brine, dried over anhydrous Na₂SO₄ and evaporated under reduced pressure. The crude was purified by silica gel chromatography (10% MeOH/DCM) to afford 482-5 (0.3 g, yield: 40%) as yellow oil.

### The Synthesis of 3-((4-(1H-imidazol-4-yl)phenoxy)methyl)-6-(ethylsulfonyl)pyridazine (482-6)

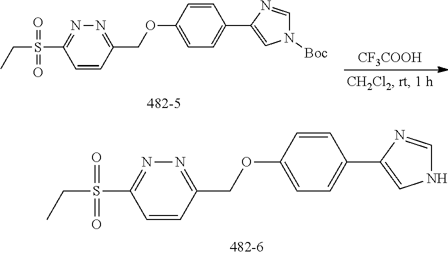 [SMILES: CCS(=O)(=O)c1ccc(COc2ccc(-c3cn(C(=O)OC(C)(C)C)cn3)cc2)nn1 ; "CF3COOH" ; CCS(=O)(=O)c1ccc(COc2ccc(-c3c[nH]cn3)cc2)nn1]

To a stirred solution of 482-5 (300 mg, 1.00 mmol) in CH₂Cl₂ (10 mL) was added TFA (5 mL). The reaction mixture was stirred at room temperature for 1 hour until the reaction was complete (by LCMS). The mixture was concentrated under reduced pressure to afford 482-6 (195 mg, crude) as yellow oil, which was used to the next step without purification.

### The Synthesis of 4-(4-((6-(ethylsulfonyl)pyridazin-3-yl)methoxy)phenyl)-N-((1-phenylpyrrolidin-3-yl)methyl)-1H-imidazole-1-carboxamide (AC2020601-0482)

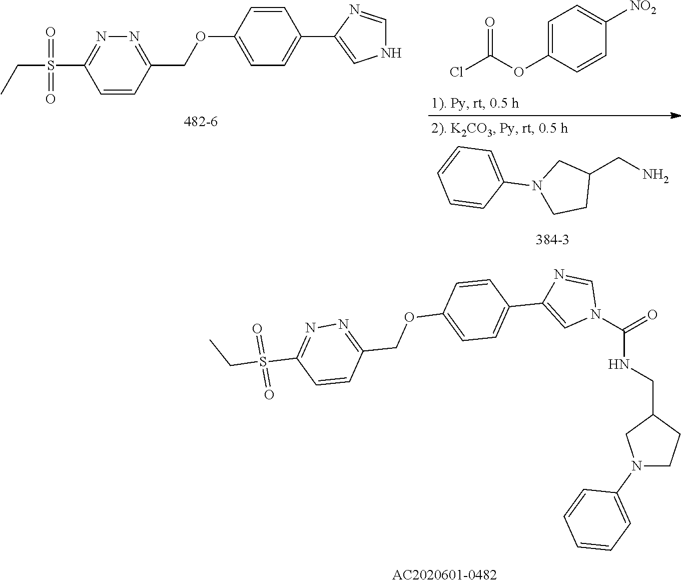 [SMILES: CCS(=O)(=O)c1ccc(COc2ccc(-c3c[nH]cn3)cc2)nn1 ; O=C(Cl)Oc1ccc([N+](=O)[O-])cc1 ; NCC1CCN(c2ccccc2)C1 ; CCS(=O)(=O)c1ccc(COc2ccc(-c3cn(C(=O)NCC4CCN(c5ccccc5)C4)cn3)cc2)nn1]

To a solution of 482-6 (114 mg, 0.33 mmol) in Py (5 mL) was added SM1 (82.0 mg, 0.4 mmol) at 0° C. and stirred for 0.5 hour. Then the reaction mixture was allowed to warm to room temperature and K₂CO₃ (138 mg, 1.0 mmol), 384-3 (71.0 mg, 0.4 mmol) was added. After 0.5 hour stirring, the reaction mixture was quenched with water, extracted with DCM (20 mL), the combined organic layer was washed with brine, dried over anhydrous Na₂SO₄ and evaporated under reduced pressure. The crude was purified by silica gel chromatography (10% MeOH/DCM) to afford AC2020601-0482 (11 mg, yield: 6.1%) as a white solid. ¹H NMR (400 MHz, DMSO-d₆) δ 8.68 (s, 1H), 8.38 (d, J=8.8 Hz, 1H), 8.31 (d, J=1.2 Hz, 1H), 8.19 (d, J=8.8 Hz, 1H), 8.06 (d, J=0.8 Hz, 1H), 7.77 (d, J=8.8 Hz, 2H), 7.12-7.17 (m, 4H), 6.52-6.60 (m, 3H), 5.61 (s, 2H), 3.66 (q, J=7.6 Hz, 2H), 3.34-3.41 (m, 3H), 3.21-3.29 (m, 2H), 3.04-3.09 (m, 1H), 2.61-2.65 (m, 1H), 2.08-2.15 (m, 1H), 1.77-1.85 (m, 1H), 1.22 (t, J=8.4 Hz, 3H). LC-MS m/z: 547.3 [M+H]⁺. LC-MS Purity (254 nm):100%; tR=2.100 min.

AC2020601-0488

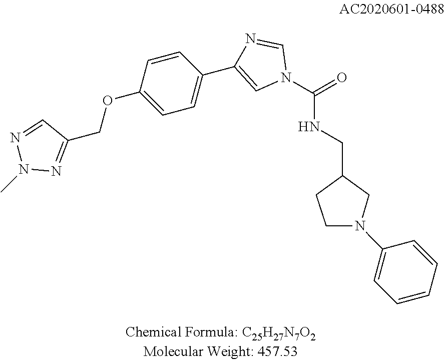 [SMILES: Cn1ncc(COc2ccc(-c3cn(C(=O)NCC4CCN(c5ccccc5)C4)cn3)cc2)n1]

### The Synthesis of methyl 2-methyl-2H-1,2,3-triazole-4-carboxylate (488-2)

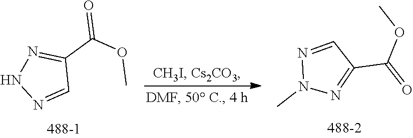

To a suspension of 488-1 (7.62 g, 60.0 mmol) in DMF (50 mL) was added CH₃I (8.5 g, 60.0 mmol) and Cs₂CO₃ (39.0 g, 120.0 mmol). The reaction mixture was allowed to warm to 50° C. for 4 hours until the reaction was complete (by LCMS). Water was added and the reaction mixture was extracted with EtOAc (80 mL), the combined organic layer was washed with brine, dried over anhydrous Na₂SO₄ and evaporated under reduced pressure. The crude was purified by silica gel chromatography (30% EA/PE) to afford 488-2 (2.6 g, yield: 32%) as white oil.

### The Synthesis of (2-methyl-2H-1,2,3-triazol-4-yl)methanol (488-3)

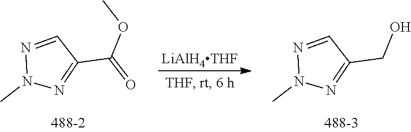

To a suspension of 488-2 (1.0 g, 7.1 mmol) in THF (10 mL) was added LiAlH₄·THF (4.3 mL). The reaction mixture was stirred at room temperature for 6 hours until the reaction was complete (by LCMS). Water was added and the reaction mixture was extracted with EtOAc (50 mL), the combined organic layer was washed with brine, dried over anhydrous Na₂SO₄ and evaporated under reduced pressure. The crude was purified by silica gel chromatography (20% EA/PE) to afford 488-3 (800 mg, yield: 91%) as white oil.

### The Synthesis of tert-butyl 4-(4-((2-methyl-2H-1,2,3-triazol-4-yl)methoxy)phenyl)-1H-imidazole-1-carboxylate (488-4)

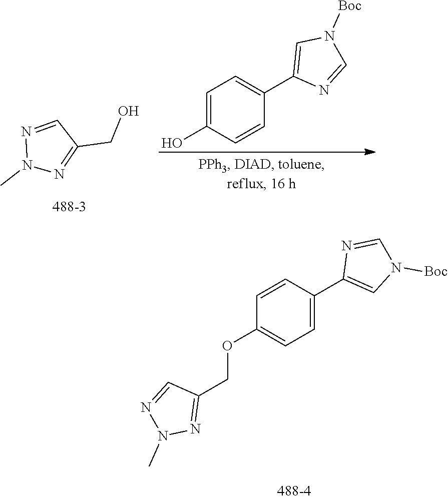

To a suspension of 488-3 (800 mg, 7.0 mmol) in Tol (15 mL) was added tert-butyl 4-(4-hydroxyphenyl)-1H-imidazole-1-carboxylate (910 mg, 3.5 mmol), PPh₃ (1.1 g, 4.2 mmol) and DIAD (920 mg, 4.55 mmol). The reaction mixture was allowed to warm to 110° C. for 16 hours until the reaction was complete (by LCMS). Water was added and the reaction mixture was extracted with EtOAc (50 mL), the combined organic layer was washed with brine, dried over anhydrous Na₂SO₄ and evaporated under reduced pressure. The crude was purified by silica gel chromatography (30% EA/PE) to afford 488-4 (500 mg, yield: 26%) as yellow oil.

### The Synthesis of 4-((4-(1H-imidazol-4-yl)phenoxy)methyl)-2-methyl-2H-1,2,3-triazole (488-5)

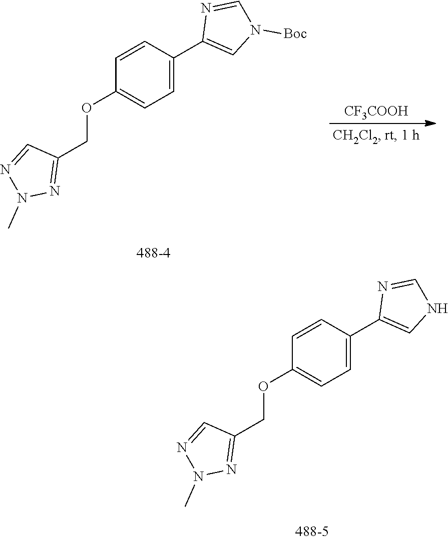 [SMILES: Cn1ncc(COc2ccc(-c3cn(C(=O)OC(C)(C)C)cn3)cc2)n1 ; "CF3COOH" ; Cn1ncc(COc2ccc(-c3c[nH]cn3)cc2)n1]

To a stirred solution of 488-4 (500 mg, 1.4 mmol) in CH₂Cl₂ (10 mL) was added TFA (10 mL). The reaction mixture was stirred at room temperature for 1 hour until the reaction was complete (by LCMS). The reaction mixture was quenched with water, extracted with DCM (20 mL), the combined organic layer was washed with brine, dried over anhydrous Na₂SO₄. The mixture was concentrated under reduced pressure to afford 488-5 (250 mg, yield: 70%) as yellow oil, which was used to the next step without purification.

### The Synthesis of 4-(4-((2-methyl-2H-1,2,3-triazol-4-yl)methoxy)phenyl)-N-((1-phenylpyrrolidin-3-yl)methyl)-1H-imidazole-1-carboxamide (AC2020601-0488)

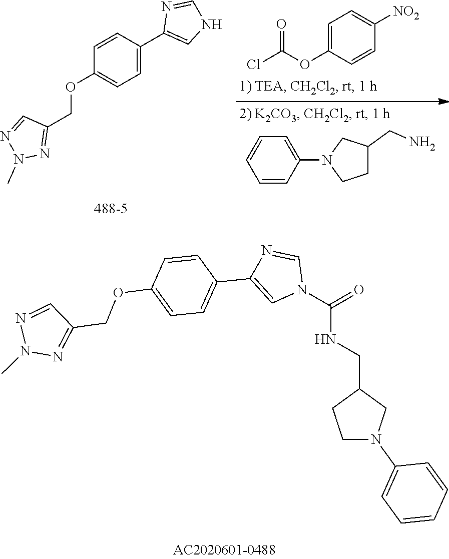 [SMILES: Cn1ncc(COc2ccc(-c3c[nH]cn3)cc2)n1 ; NCC1CCN(c2ccccc2)C1 ; O=C(Cl)Oc1ccc([N+](=O)[O-])cc1 ; Cn1ncc(COc2ccc(-c3cn(C(=O)NCC4CCN(c5ccccc5)C4)cn3)cc2)n1]

To a solution of 488-5 (250 mg, 1.0 mmol) in DCM (5 mL) was added 4-nitrophenyl carbonochloridate (300 mg, 1.5 mmol) and Et₃N (5 mL) at room temperature and stirred for 1 hour. Then (1-phenylpyrrolidin-3-yl)methanamine (352 mg, 2.0 mmol) and K₂CO₃ (276 mg, 2.0 mmol) was added. After 1 hour stirring, the reaction mixture was quenched with water, extracted with DCM (20 mL), the combined organic layer was washed with brine, dried over anhydrous Na₂SO₄ and evaporated under reduced pressure. The crude was purified by silica gel chromatography (5% MeOH/DCM) to afford AC2020601-0488 (60 mg, yield: 13%) as a white solid. ¹H NMR (400 MHz, DMSO-d₆) δ 8.65 (t, J=5.6 Hz, 1H), 8.30 (d, J=1.6 Hz, 1H), 8.03 (d, J=1.2 Hz, 1H), 7.85 (s, 1H), 7.74 (d, J=8.8 Hz, 2H), 7.15 (t, J=8.4 Hz, 2H), 7.06 (d, J=8.8 Hz, 2H), 6.58 (t, J=7.2 Hz, 1H) 6.53 (d, J=8.0 Hz, 2H), 5.16 (s, 2H), 4.16 (s, 3H), 3.34-3.40 (m, 4H), 3.21-3.27 (m, 1H), 3.05-3.09 (m, 1H), 2.61-2.66 (m, 1H), 2.10-2.15 (m, 1H), 1.78-1.83 (m, 1H). LC-MS m/z: 458.4 [M+H]⁺. HPLC Purity (254 nm): 99.99%; t_{R}=3.690 min.

AC2020601-0489

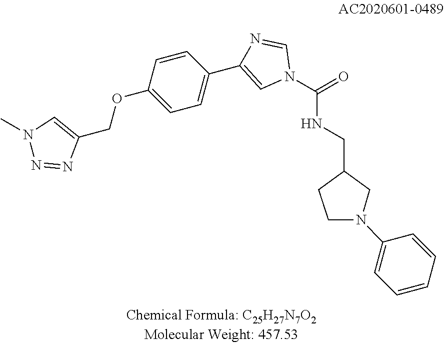 [SMILES: Cn1cc(COc2ccc(-c3cn(C(=O)NCC4CCN(c5ccccc5)C4)cn3)cc2)nn1]

### The Synthesis of methyl 1-methyl-1H-1,2,3-triazole-4-carboxylate (489-2)

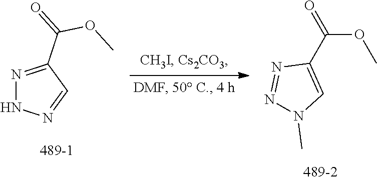

To a suspension of 489-1 (7.62 g, 60.0 mmol) in DMF (30 mL) was added CH₃I (8.5 g, 60.0 mmol) and Cs₂CO₃ (39.0 g, 120.0 mmol). The reaction mixture was allowed to warm to 50° C. for 4 hours until the reaction was complete (by LCMS). Water was added and the reaction mixture was extracted with EtOAc (50 mL), the combined organic layer was washed with brine, dried over anhydrous Na₂SO₄ and evaporated under reduced pressure. The crude was purified by silica gel chromatography (15% PE/EA) to afford 489-2 (1.3 g, yield: 15.3%) as white oil.

### The Synthesis of (1-methyl-1H-1,2,3-triazol-4-yl)methanol (489-3)

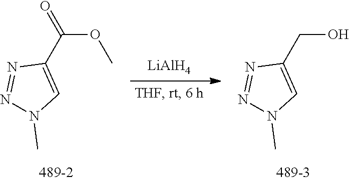

To a suspension of 489-2 (800 mg, 5.7 mmol) in THF (10 mL) was added LiAlH₄·THF (3.4 mL). The reaction mixture was stirred at room temperature for 6 hours until the reaction was complete (by LCMS). Water was added and the reaction mixture was extracted with EtOAc (50 mL), the combined organic layer was washed with brine, dried over anhydrous Na₂SO₄ and evaporated under reduced pressure. The crude was purified by silica gel chromatography (15% PE/EA) to afford 489-3 (800 mg, yield: 45.2%) as white oil.

### The Synthesis of tert-butyl 4-(4-((1-methyl-1H-1,2,3-triazol-4-yl)methoxy)phenyl)-1H-imidazole-1-carboxylate (489-4)

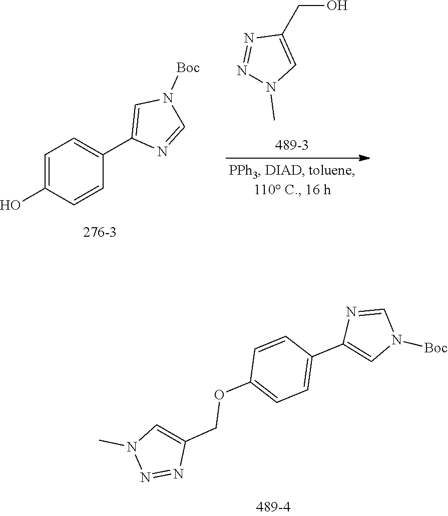

To a suspension of 489-3 (500 mg, 4.4 mmol) in THF (20 mL) was added 276-3 (572 mg, 2.2 mmol), PPh₃ (681 mg, 2.6 mmol) and DIAD (578 mg, 2.86 mmol). The reaction mixture was allowed to warm to 110° C. for 16 hours until the reaction was complete (by LCMS). Water was added and the reaction mixture was extracted with EtOAc (50 mL), the combined organic layer was washed with brine, dried over anhydrous Na₂SO₄ and evaporated under reduced pressure. The crude was purified by silica gel chromatography (45% EA/PE) to afford 489-4 (300 mg, yield: 32.4%) as yellow oil.

### The Synthesis of 4-((4-(1H-imidazol-4-yl)phenoxy)methyl)-1-methyl-1H-1,2,3-triazole (489-5)

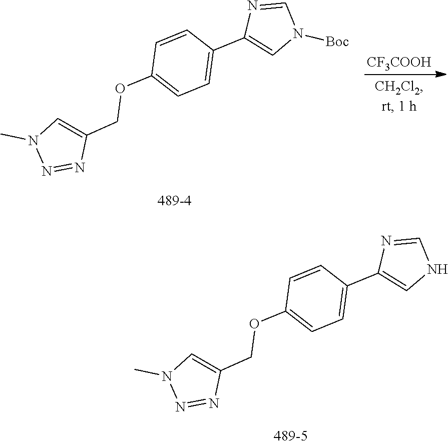 [SMILES: Cn1cc(COc2ccc(-c3cn(C(=O)OC(C)(C)C)cn3)cc2)nn1 ; "CF3COOH" ; Cn1cc(COc2ccc(-c3c[nH]cn3)cc2)nn1]

To a stirred solution of 489-4 (300 mg, 0.84 mmol) in CH₂Cl₂ (10 mL) was added TFA (10 mL). The reaction mixture was stirred at room temperature for 1 hour until the reaction was complete (by LCMS). The reaction mixture was quenched with water, extracted with DCM (20 mL), the combined organic layer was washed with brine, dried over anhydrous Na₂SO₄. The mixture was concentrated under reduced pressure to afford 489-5 (100 mg, yield: 64.1%) as yellow oil, which was used to the next step without purification.

### The Synthesis of 4-(4-((1-methyl-1H-1,2,3-triazol-4-yl)methoxy)phenyl)-N-((1-phenylpyrrolidin-3-yl)methyl)-1H-imidazole-1-carboxamide (AC2020601-0489)

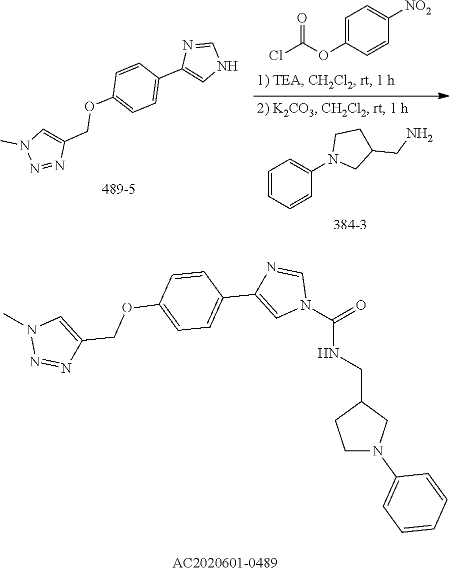 [SMILES: Cn1cc(COc2ccc(-c3c[nH]cn3)cc2)nn1 ; NCC1CCN(c2ccccc2)C1 ; O=C(Cl)Oc1ccc([N+](=O)[O-])cc1 ; Cn1cc(COc2ccc(-c3cn(C(=O)NCC4CCN(c5ccccc5)C4)cn3)cc2)nn1]

To a solution of 489-5 (100 mg, 0.4 mmol) in DCM (5 mL) was added 4-nitrophenyl carbonochloridate (121 mg, 0.6 mmol) and Et₃N (5 mL) at room temperature and stirred for 1 hour. Then (1-phenylpyrrolidin-3-yl)methanamine (384-3, 141 mg, 0.8 mmol) and K₂CO₃ (110 mg, 0.8 mmol) was added. After 1 hour stirring, the reaction mixture was quenched with water, extracted with DCM (20 mL), the combined organic layer was washed with brine, dried over anhydrous Na₂SO₄ and evaporated under reduced pressure. The crude was purified by silica gel chromatography (5% MeOH/DCM) to afford AC2020601-0489 (25.18 mg, yield: 18.4%) as white solid. ¹H NMR (400 MHz, DMSO-d₆) δ 8.65 (t, J=5.6 Hz, 1H), 8.30 (d, J=1.2 Hz, 1H), 8.18 (s, 1H), 8.03 (d, J=1.2 Hz, 1H), 7.73 (d, J=8.8 Hz, 2H), 7.13-7.17 (m, 2H), 7.07 (d, J=8.8 Hz, 2H), 6.58 (t, J=7.2 Hz, 1H), 6.53 (d, J=7.6 Hz, 2H), 5.16 (s, 2H), 4.05 (s, 3H), 3.34-3.40 (m, 4H), 3.21-3.27 (m, 1H), 3.05-3.09 (m, 1H), 2.66-2.66 (m, 1H), 2.10-2.15 (m, 1H), 1.78-1.83 (m, 1H). LC-MS m/z: 458.4 [M+H]⁺. HPLC Purity (254 nm): 95.84%; t_{R}=3.41 min.

AC2020601-0491

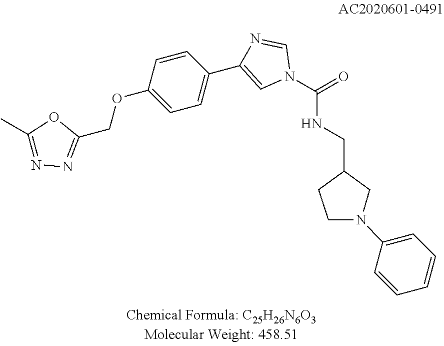 [SMILES: Cc1nnc(COc2ccc(-c3cn(C(=O)NCC4CCN(c5ccccc5)C4)cn3)cc2)o1]

### The Synthesis of tert-butyl 4-(4-((5-methyl-1,3,4-oxadiazol-2-yl)methoxy)phenyl)-1H-imidazole-1-carboxylate (491-2)

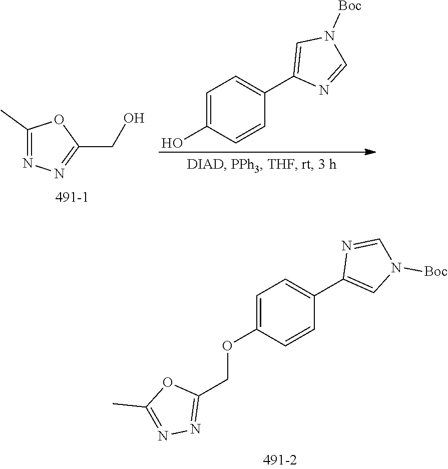

To a suspension of 491-1 (460 mg, 4.0 mmol) in THF (15 mL) was added tert-butyl 4-(4-hydroxyphenyl)-1H-imidazole-1-carboxylate (520 mg, 2.0 mmol), PPh₃ (628 mg, 2.4 mmol) and DIAD (525 mg, 2.6 mmol). The reaction mixture was stirred at room temperature for 3 hours until the reaction was complete (by LCMS). Water was added and the reaction mixture was extracted with EtOAc (50 mL), the combined organic layer was washed with brine, dried over anhydrous Na₂SO₄ and evaporated under reduced pressure. The crude was purified by silica gel chromatography (30% EA/PE) to afford 491-2 (300 mg, yield: 38%) as yellow oil.

### The Synthesis of 5-((4-(1H-imidazol-4-yl)phenoxy)methyl)-2-(methylsulfonyl)pyridine (491-3)

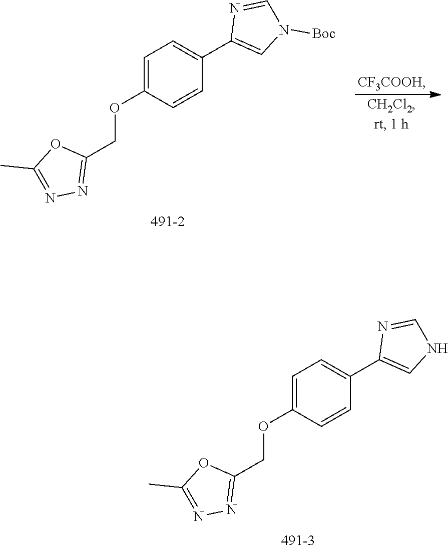 [SMILES: Cc1nnc(COc2ccc(-c3cn(C(=O)OC(C)(C)C)cn3)cc2)o1 ; "CF3COOH," ; "CH2Cl2,rt, 1 h" ; Cc1nnc(COc2ccc(-c3c[nH]cn3)cc2)o1]

To a stirred solution of 491-2 (300 mg, 0.84 mmol) in CH₂Cl₂ (10 mL) was added TFA (10 mL). The reaction mixture was stirred at room temperature for 1 hour until the reaction was complete (by LCMS). The reaction mixture was quenched with water, extracted with DCM (20 mL), the combined organic layer was washed with brine, dried over anhydrous Na₂SO₄. The mixture was concentrated under reduced pressure to afford 491-3 (150 mg, yield: 65%) as yellow oil, which was used to the next step without purification.

### The Synthesis of 4-(4-((5-methyl-1,3,4-oxadiazol-2-yl)methoxy)phenyl)-N-((1-phenylpyrrolidin-3-yl)methyl)-1H-imidazole-1-carboxamide (AC2020601-0491)

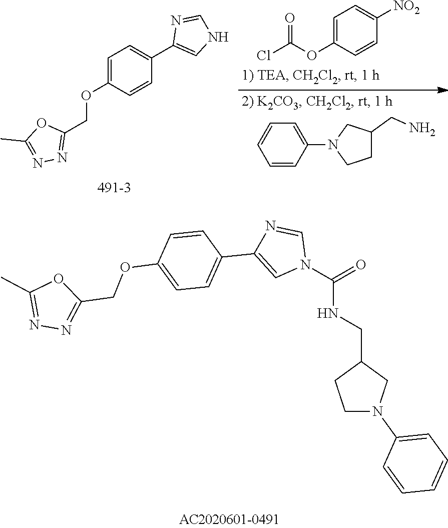 [SMILES: Cc1nnc(COc2ccc(-c3c[nH]cn3)cc2)o1 ; NCC1CCN(c2ccccc2)C1 ; O=C(Cl)Oc1ccc([N+](=O)[O-])cc1 ; Cc1nnc(COc2ccc(-c3cn(C(=O)NCC4CCN(c5ccccc5)C4)cn3)cc2)o1]

To a solution of 491-3 (150 mg, 0.6 mmol) in DCM (5 mL) was added 4-nitrophenyl carbonochloridate (184 mg, 0.9 mmol) and Et₃N (5 mL) at room temperature and stirred for 1 hour. Then (1-phenylpyrrolidin-3-yl)methanamine (211 mg, 1.2 mmol) and K₂CO₃ (156 mg, 1.2 mmol)was added. After 1 hour stirring, the reaction mixture was quenched with water, extracted with DCM (20 mL), the combined organic layer was washed with brine, dried over anhydrous Na₂SO₄ and evaporated under reduced pressure. The crude was purified by silica gel chromatography (5% MeOH/DCM) to afford AC2020601-0491 (6.0 mg, yield: 1.4%) as a white solid. ¹H NMR (400 MHz, DMSO-d₆) δ 8.67 (t, J=5.6 Hz, 1H), 8.31 (d, J=1.2 Hz, 1H), 8.06 (d, J=0.8 Hz, 1H), 7.76 (d, J=8.8 Hz, 2H), 7.15 (t, J=7.6 Hz, 2H), 7.10 (d, J=9.2 Hz, 2H), 6.58 (t, J=7.2 Hz, 1H), 6.53 (d, J=8.0 Hz, 2H), 5.38 (s, 2H), 3.36-3.41 (m, 4H), 3.23-3.27 (m, 1H), 3.05-3.09 (m, 1H), 2.61-2.67 (m, 1H), 2.53 (s, 3H), 2.10-2.15 (m, 1H), 1.78-1.83 (m, 1H). LC-MS m/z: 459.3 [M+H]⁺. HPLC Purity (254 nm): 96.13%; t_{R}=3.492 min.

AC2020601-0494

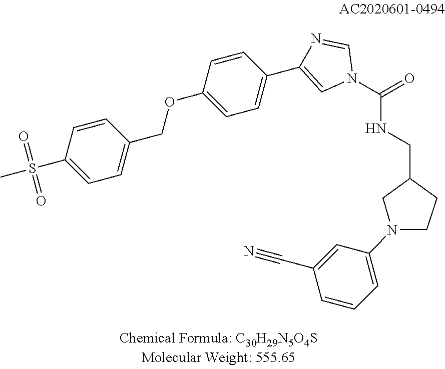 [SMILES: CS(=O)(=O)c1ccc(COc2ccc(-c3cn(C(=O)NCC4CCN(c5cccc(C#N)c5)C4)cn3)cc2)cc1]

### The Synthesis of tert-butyl (1-(3-cyanophenyl)pyrrolidin-3-yl)methylcarbamate (494-2)

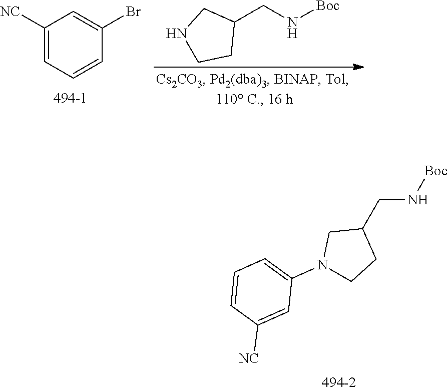 [SMILES: N#Cc1cccc(Br)c1 ; CC(C)(C)OC(=O)NCC1CCNC1 ; CC(C)(C)OC(=O)NCC1CCN(c2cccc(C#N)c2)C1]

To a suspension of 494-1 (364 mg, 2.0 mmol) in Tol (15 mL) was added tert-butyl pyrrolidin-3-ylmethylcarbamate (200 mg, 1.0 mmol), Cs₂CO₃ (650 mg, 2.0 mmol), Pd₂(dba)₃ (57 mg, 0.1 mmol) and BINAP (62 mg, 0.1 mmol). The reaction mixture was allowed to warm to 110° C. for 16 hours until the reaction was complete (by LCMS). Water was added and the reaction mixture was extracted with EtOAc (50 mL), the combined organic layer was washed with brine, dried over anhydrous Na₂SO₄ and evaporated under reduced pressure. The crude was purified by silica gel chromatography (40% EA/PE) to afford 494-2 (300 mg, yield: 50%) as yellow oil.

### The Synthesis of 3-(3-(aminomethyl)pyrrolidin-1-yl)benzonitrile (494-3)

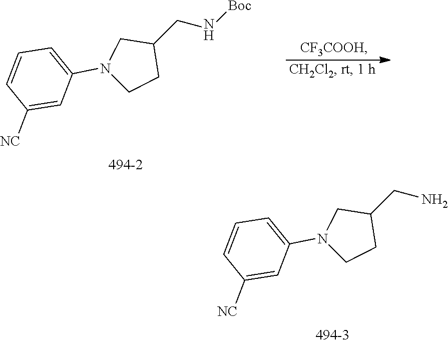 [SMILES: CC(C)(C)OC(=O)NCC1CCN(c2cccc(C#N)c2)C1 ; "CF3COOH," ; "CH2Cl2, rt, 1 h" ; N#Cc1cccc(N2CCC(CN)C2)c1]

To a stirred solution of 484-2 (300 mg, 1.0 mmol) in CH₂Cl₂ (10 mL) was added TFA (10 mL). The reaction mixture was stirred at room temperature for 1 hour until the reaction was complete (by LCMS). The reaction mixture was quenched with water, extracted with DCM (20 mL), the combined organic layer was washed with brine, dried over anhydrous Na₂SO₄. The mixture was concentrated under reduced pressure to afford 494-3 (150 mg, yield: 68%) as yellow oil, which was used to the next step without purification.

### The Synthesis of N-((1-(3-cyanophenyl)pyrrolidin-3-yl)methyl)-4-(4-(4-(methylsulfonyl)benzyloxy)phenyl)-1H-imidazole-1-carboxamide (AC2020601-0494)

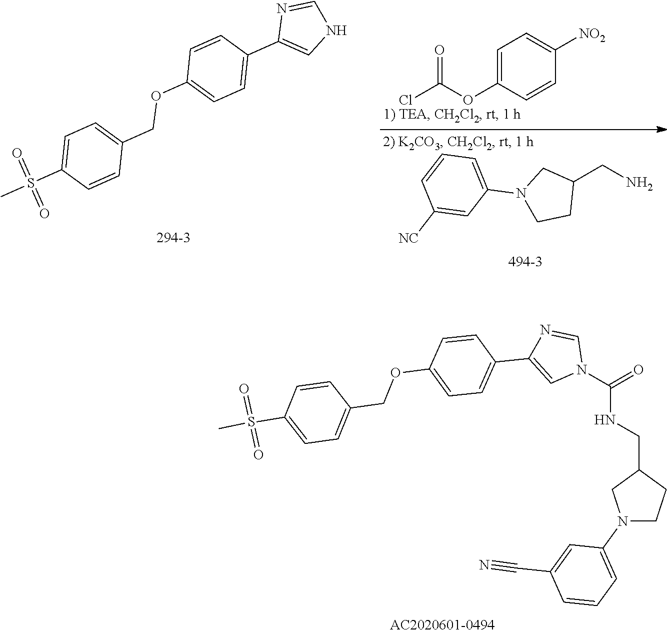 [SMILES: CS(=O)(=O)c1ccc(COc2ccc(-c3c[nH]cn3)cc2)cc1 ; O=C(Cl)Oc1ccc([N+](=O)[O-])cc1 ; N#Cc1cccc(N2CCC(CN)C2)c1 ; CS(=O)(=O)c1ccc(COc2ccc(-c3cn(C(=O)NCC4CCN(c5cccc(C#N)c5)C4)cn3)cc2)cc1]

To a solution of 294-3 (121 mg, 0.37 mmol) in DCM (5 mL) was added 4-nitrophenyl carbonochloridate (112 mg, 0.55 mmol) and Et₃N (5 mL) at room temperature and stirred for 1 hour. Then 494-3 (150 mg, 0.74 mmol) and K₂CO₃ (102 mg, 0.74 mmol) was added. After 1 hour stirring, the reaction mixture was quenched with water, extracted with DCM (20 mL), the combined organic layer was washed with brine, dried over anhydrous Na₂SO₄ and evaporated under reduced pressure. The crude was purified by silica gel chromatography (5% MeOH/DCM) to afford AC2020601-0494 (13.04 mg, yield: 4.6%) as a white solid. ¹H NMR (400 MHz, DMSO-d₆) δ 8.65 (t, J=5.6 Hz, 1H), 8.29 (d, J=1.2 Hz, 1H), 8.02 (d, J=1.2 Hz, 1H), 7.96 (d, J=8.4 Hz, 2H), 7.74 (t, J=8.8 Hz, 4H), 7.33 (t, J=8.0 Hz, 1H), 7.07 (d, J=8.8 Hz, 2H), 6.96 (d, J=7.6 Hz, 1H), 6.83-6.87 (m, 2H), 5.28 (s, 2H), 3.35-3.45 (m, 4H), 3.27-3.29 (m, 1H), 3.22 (s, 3H), 3.11 (dd, J=9.6 Hz, J=6.0 Hz, 1H), 2.61-2.68 (m, 1H), 2.12-2.16 (m, 1H), 1.80-1.85 (m, 1H). LC-MS m/z: 329.2 (fraction). HPLC Purity (254 nm): 90.96%; t_{R}=3.662 min.

AC2020601-0496

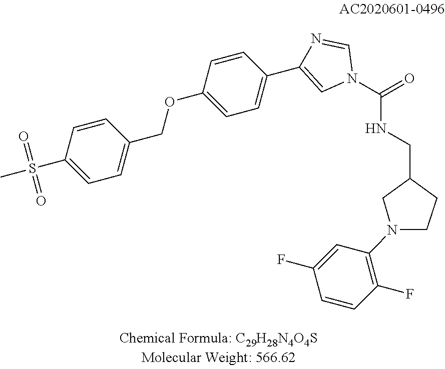 [SMILES: CS(=O)(=O)c1ccc(COc2ccc(-c3cn(C(=O)NCC4CCN(c5cc(F)ccc5F)C4)cn3)cc2)cc1]

### The Synthesis of tert-butyl (1-(2,5-difluorophenyl)pyrrolidin-3-yl)methylcarbamate (496-2)

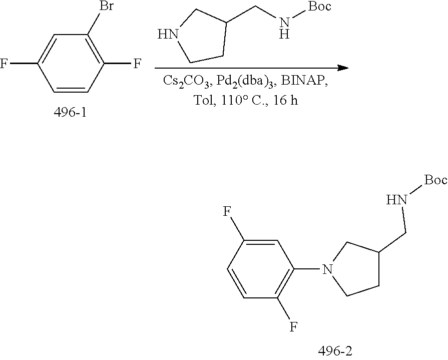

To a suspension of 496-1 (386 mg, 2.0 mmol) in Tol (10 mL) was added tert-butyl pyrrolidin-3-ylmethylcarbamate (200 mg, 1.0 mmol), Cs₂CO₃ (650 mg, 2.0 mmol), Pd₂(dba)₃ (57 mg, 0.1 mmol) and BINAP (62 mg, 0.1 mmol). The reaction mixture was allowed to warm to 100° C. for 16 hours until the reaction was complete (by LCMS). Water was added and the reaction mixture was extracted with EtOAc (50 mL), the combined organic layer was washed with brine, dried over anhydrous Na₂SO₄ and evaporated under reduced pressure. The crude was purified by silica gel chromatography (30% EA/PE) to afford 496-2 (300 mg, yield: 51.2%) as yellow oil.

### The Synthesis of (1-(2,5-difluorophenyl)pyrrolidin-3-yl)methanamine (496-3)

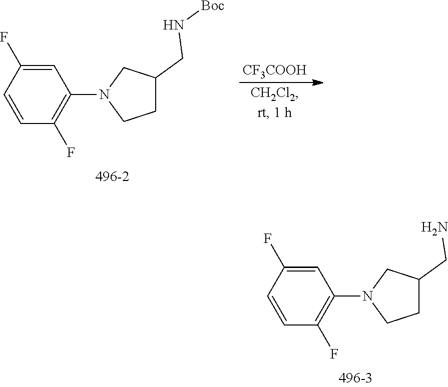 [SMILES: CC(C)(C)OC(=O)NCC1CCN(c2cc(F)ccc2F)C1 ; "CF3COOH" ; "CH2Cl2,rt, 1 h" ; NCC1CCN(c2cc(F)ccc2F)C1]

To a stirred solution of 496-2 (300 mg, 0.96 mmol) in CH₂Cl₂ (10 mL) was added TFA (10 mL). The reaction mixture was stirred at room temperature for 1 hour until the reaction was complete (by LCMS). The reaction mixture was quenched with water, extracted with DCM (20 mL), the combined organic layer was washed with brine, dried over anhydrous Na₂SO₄. The mixture was concentrated under reduced pressure to afford 496-3 (150 mg, yield: 73.9%) as yellow oil, which was used to the next step without purification.

### The Synthesis of N-((1-(2,5-difluorophenyl)pyrrolidin-3-yl)methyl)-4-(4-(4-(methylsulfonyl)benzyloxy)phenyl)-1H-imidazole-1-carboxamide (AC2020601-0496)

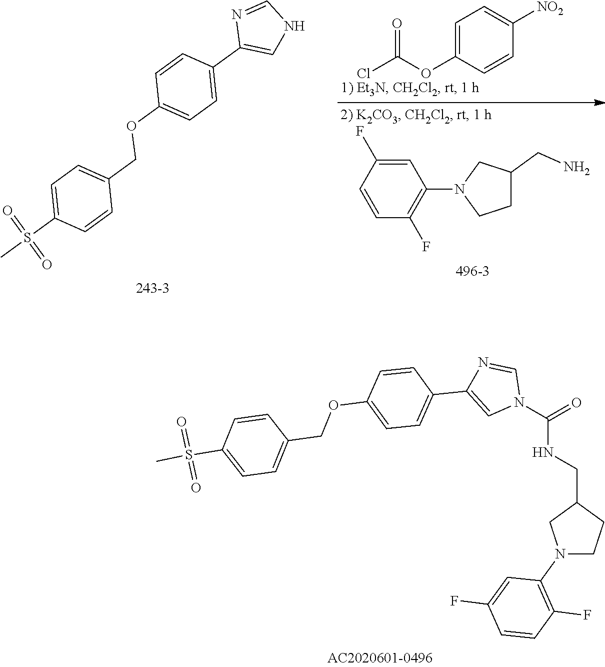 [SMILES: CS(=O)(=O)c1ccc(COc2ccc(-c3c[nH]cn3)cc2)cc1 ; NCC1CCN(c2cc(F)ccc2F)C1 ; O=C(Cl)Oc1ccc([N+](=O)[O-])cc1 ; CS(=O)(=O)c1ccc(COc2ccc(-c3cn(C(=O)NCC4CCN(c5cc(F)ccc5F)C4)cn3)cc2)cc1]

To a solution of 243-3 (114 mg, 0.35 mmol) in DCM (5 mL) was added 4-nitrophenyl carbonochloridate (105 mg, 0.52 mmol) and Et₃N (5 mL) at room temperature and stirred for 1 hour. Then 496-3 (150 mg, 0.7 mmol) and K₂CO₃ (96 mg, 0.7 mmol) was added. After 1 hour stirring, the reaction mixture was quenched with water, extracted with DCM (20 mL), the combined organic layer was washed with brine, dried over anhydrous Na₂SO₄ and evaporated under reduced pressure. The crude was purified by silica gel chromatography (5% MeOH/DCM) to afford AC2020601-0496 (10.01 mg, yield: 3.4%) as white solid. ¹H NMR (400 MHz, DMSO-d₆) δ 8.63 (t, J=5.6 Hz, 1H), 8.29 (d, J=0.8 Hz, 1H), 8.02 (s, 1H), 7.96 (d, J=8.0 Hz, 2H), 7.73 (t, J=8.4 Hz, 4H), 7.02-7.08 (m, 3H), 6.47-6.52 (m, 1H), 6.38-6.43 (m, 1H), 5.28 (s, 2H), 3.44-3.51 (m, 2H), 3.35-3.39 (m, 3H), 3.19-3.22 (m, 4H), 2.54-2.59 (m, 1H), 2.05-2.09 (m, 1H), 1.73-1.78 (m, 1H). LC-MS m/z: 329.2 (fraction), HPLC Purity (254 nm): 78.42%; t_{R}=1.959 min.

AC2020601-0497

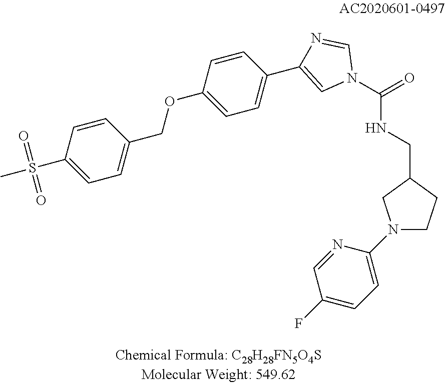 [SMILES: CS(=O)(=O)c1ccc(COc2ccc(-c3cn(C(=O)NCC4CCN(c5ccc(F)cn5)C4)cn3)cc2)cc1]

### The Synthesis of tert-butyl ((1-(3-chlorophenyl)azetidin-3-yl)methyl)carbamate (497-2)

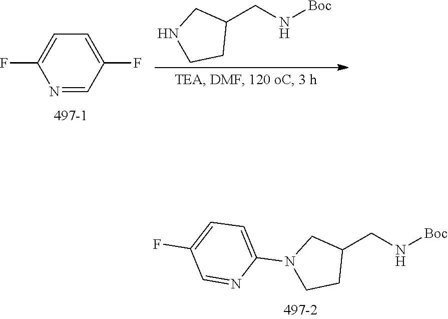

A mixture of 497-1 (1.15 g, 10.00 mmol), tert-butyl (pyrrolidin-3-ylmethyl)carbamate (2.40 g, 11.99 mmol),TEA (2.02 g, 19.99 mmol) in DMF (10 mL) was stirred at 120° C. for 3 hours under N₂ until the reaction was complete (by LCMS). The reaction mixture was quenched with water and extracted with EtOAc (50 mL), the combined organic layer was washed with brine, dried over anhydrous Na₂SO₄ and evaporated under reduced pressure. The crude was purified by silica gel chromatography (25% EA/PE) to afford 497-2 (2.85 g, yield: 97%) as yellow oil.

### The Synthesis of (1-(5-fluoropyridin-2-yl)pyrrolidin-3-yl)methanamine (497-3)

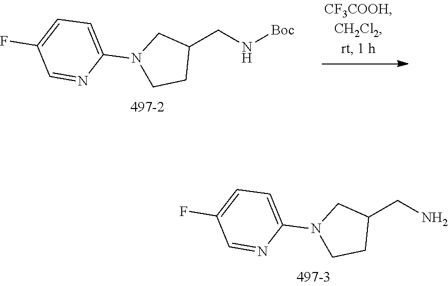 [SMILES: CC(C)(C)OC(=O)NCC1CCN(c2ccc(F)cn2)C1 ; NCC1CCN(c2ccc(F)cn2)C1]

To a stirred solution of 497-2 (295 mg, 1.00 mmol) in CH₂Cl₂ (10 mL) was added TFA (5 mL). The reaction mixture was stirred at room temperature for 2 hours until the reaction was complete (by LCMS). The mixture was concentrated under reduced pressure to afford 497-3 (180 mg, crude) as yellow oil, which was used to the next step without purification.

### The Synthesis of N-((1-(5-fluoropyridin-2-yl)pyrrolidin-3-yl)methyl)-4-(4-((4-(methylsulfonyl)benzyl)oxy)phenyl)-1H-imidazole-1-carboxamide (AC2020601-0497)

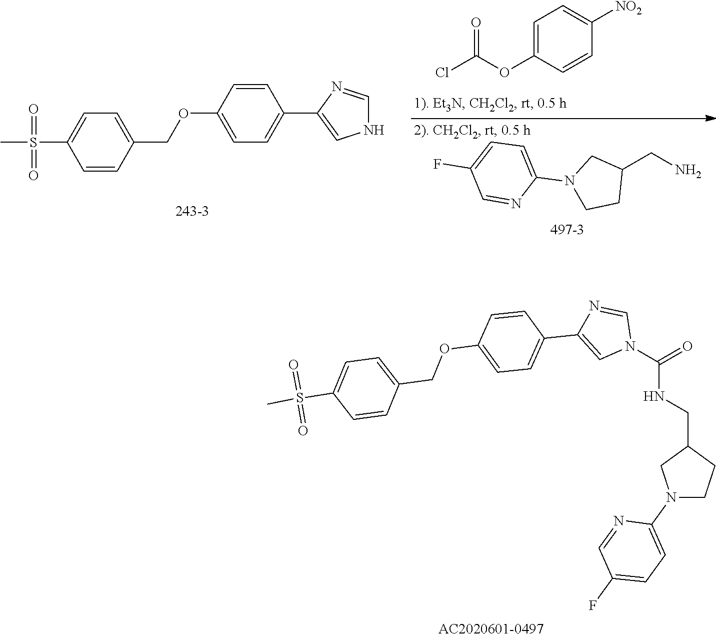 [SMILES: O=C(Cl)Oc1ccc([N+](=O)[O-])cc1 ; CS(=O)(=O)c1ccc(COc2ccc(-c3c[nH]cn3)cc2)cc1 ; NCC1CCN(c2ccc(F)cn2)C1 ; CS(=O)(=O)c1ccc(COc2ccc(-c3cn(C(=O)NCC4CCN(c5ccc(F)cn5)C4)cn3)cc2)cc1]

To a solution of 243-3 (250 mg, 0.76 mmol) in DCM (5 mL) was added 4-nitrophenyl carbonochloridate (169 mg, 0.84 mmol) and Et₃N (2 mL) at 0° C. and stirred for 0.5 hour. Then the reaction mixture was allowed to warm to room temperature and 497-3 (149 mg, 0.76 mmol) was added. After 0.5 hour stirring, the reaction mixture was quenched with water, extracted with DCM (20 mL), the combined organic layer was washed with brine, dried over anhydrous Na₂SO₄ and evaporated under reduced pressure. The crude was purified by silica gel chromatography (5% MeOH/DCM) to afford AC2020601-0497 (47 mg, yield: 11.2%) as a white solid. ¹H NMR (400 MHz, DMSO-d₆) δ 8.67 (t, J=5.6 Hz, 1H), 8.29 (d, J=0.8 Hz, 1H), 8.03-8.04 (m, 2H), 7.96 (d, J=8.4 Hz, 2H), 7.72-7.76 (m, 4H), 7.44-7.49 (m, 1H), 7.07 (d, J=8.8 Hz, 2H), 6.45 (dd, J=9.2 Hz, 3.2 Hz, 1H), 5.28 (s, 2H), 3.45-3.59 (m, 2H), 3.32-3.41 (m, 3H), 3.23 (s, 3H), 3.16-3.21 (m, 1H), 2.57-2.62 (m, 1H), 2.09-2.13 (m, 1H), 1.78-1.83 (m, 1H). LC-MS m/z: 550.3 [M+H]⁺. HPLC Purity (254 nm):100%; t_{R}=3.404 min.

AC2020601-0498

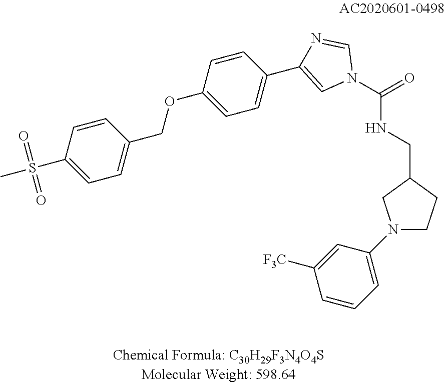 [SMILES: CS(=O)(=O)c1ccc(COc2ccc(-c3cn(C(=O)NCC4CCN(c5cccc(C(F)(F)F)c5)C4)cn3)cc2)cc1]

### The Synthesis of tert -butyl ((1-(3-(trifluoromethyl)phenyl)pyrrolidin-3-yl)methyl)carbamate (498-2)

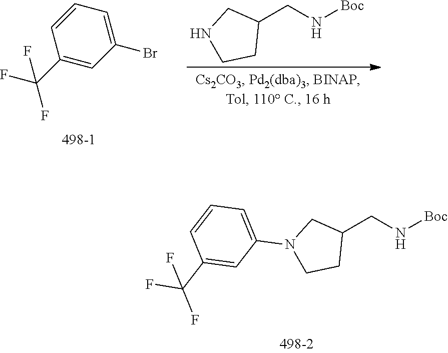 [SMILES: FC(F)(F)c1cccc(Br)c1 ; CC(C)(C)OC(=O)NCC1CCNC1 ; CC(C)(C)OC(=O)NCC1CCN(c2cccc(C(F)(F)F)c2)C1]

A mixture of 498-1 (500 mg, 2.22 mmol), tert-butyl (pyrrolidin-3-ylmethyl)carbamate (445 mg, 2.22 mmol), Pd₂(dba)₃ (20 mg, 0.02 mmol), BINAP (28 mg, 0.04 mmol) and Cs₂CO₃ (1.45 g, 4.44 mmol) in Tol (10 mL) was stirred at 110° C. overnight under N₂. The reaction mixture was quenched with water and extracted with EtOAc (50 mL), the combined organic layer was washed with brine, dried over anhydrous Na₂SO₄ and evaporated under reduced pressure. The crude was purified by silica gel chromatography (25% EA/PE) to afford 498-2 (520 mg, yield: 68%) as yellow oil.

### The Synthesis of (1-(3-(trifluoromethyl)phenyl)pyrrolidin-3-yl)methanamine (498-3)

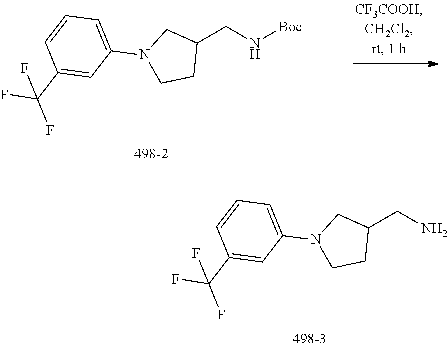 [SMILES: CC(C)(C)OC(=O)NCC1CCN(c2cccc(C(F)(F)F)c2)C1 ; NCC1CCN(c2cccc(C(F)(F)F)c2)C1]

To a stirred solution of 498-2 (344 mg, 1.00 mmol) in CH₂Cl₂ (10 mL) was added TFA (5 mL). The reaction mixture was stirred at room temperature for 1 hour until the reaction was complete (by LCMS). The mixture was concentrated under reduced pressure to afford 498-3 (235 mg, crude) as yellow oil, which was used to the next step without purification.

### The Synthesis of 4-(4-((4-(methylsulfonyl)benzyl)oxy)phenyl)-N-((1-(3-(trifluoromethyl)phenyl)pyrrolidin-3-yl)methyl)-1H-imidazole-1-carboxamide (AC2020601-0498)

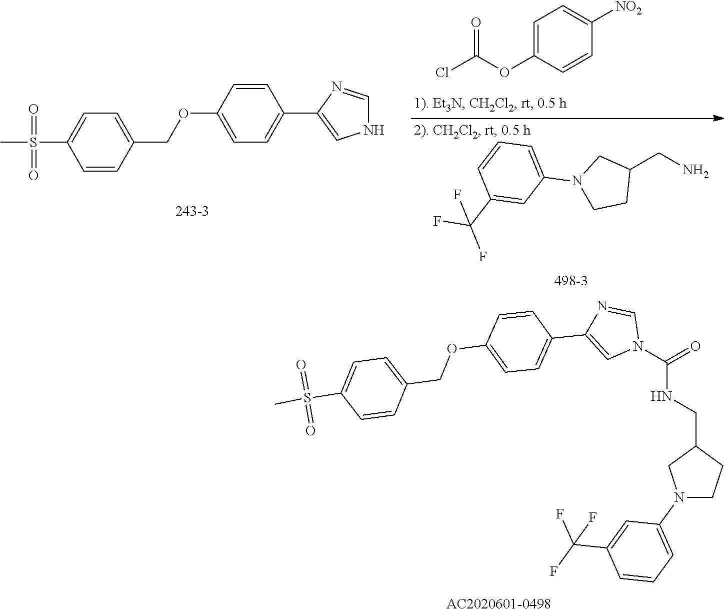 [SMILES: O=C(Cl)Oc1ccc([N+](=O)[O-])cc1 ; CS(=O)(=O)c1ccc(COc2ccc(-c3c[nH]cn3)cc2)cc1 ; NCC1CCN(c2cccc(C(F)(F)F)c2)C1 ; CS(=O)(=O)c1ccc(COc2ccc(-c3cn(C(=O)NCC4CCN(c5cccc(C(F)(F)F)c5)C4)cn3)cc2)cc1]

To a solution of 243-3 (150 mg, 0.46 mmol) in DCM (5 mL) was added 4-nitrophenyl carbonochloridate (101 mg, 0.50 mmol) and Et₃N (2 mL) at 0° C. and stirred for 0.5 hour. Then the reaction mixture was allowed to warm to room temperature and 498-3 (112 mg, 0.46 mmol) was added. After 0.5 hour stirring, the reaction mixture was quenched with water, extracted with DCM (20 mL), the combined organic layer was washed with brine, dried over anhydrous Na₂SO₄ and evaporated under reduced pressure. The crude was purified by silica gel chromatography (5% MeOH/DCM) to afford AC2020601-0498 (10 mg, yield: 3.6%) as a white solid. ¹H NMR (400 MHz, DMSO-d₆) δ 8.64 (t, J=7.0 Hz, 1H), 8.30 (d, J=0.8 Hz, 1H), 8.03 (d, J=1.2 Hz, 1H), 7.96 (d, J=8.0 Hz, 2H), 7.72-7.76 (m, 4H), 7.34-7.38 (m, 1H), 7.07 (d, J=8.4 Hz, 2H), 6.86-6.89 (m, 1H), 6.79-6.81 (m, 1H), 6.72 (s, 1H), 5.28 (s, 2H), 3.37-3.48 (m, 5H), 3.22 (s, 3H), 3.11-3.15 (m, 1H), 2.63-2.67 (m, 1H), 2.12-2.17 (m, 1H), 1.81-1.86 (m, 1H). LC-MS m/z: 1197.3 [2M+H]⁺. HPLC Purity (254 nm):94.22%; t_{R} 4.383 min.

AC2020601-0499

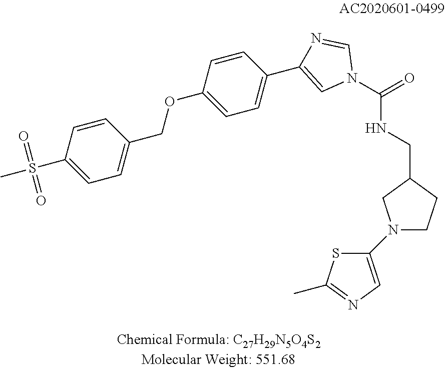 [SMILES: Cc1ncc(N2CCC(CNC(=O)n3cnc(-c4ccc(OCc5ccc(S(C)(=O)=O)cc5)cc4)c3)C2)s1]

### The Synthesis of tert-butyl ((1-(2-methylthiazol-5-yl)pyrrolidin-3-yl)methyl)carbamate (499-2)

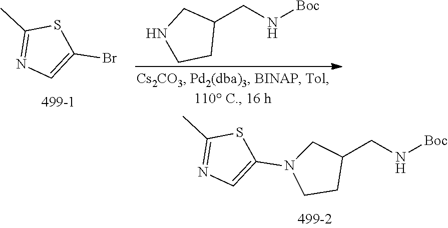

A mixture of 499-1 (395 mg, 2.22 mmol), tert-butyl (pyrrolidin-3-ylmethyl)carbamate (445 mg, 2.22 mmol), Pd₂(dba)₃ (20 mg, 0.02 mmol), BINAP (28 mg, 0.04 mmol) and Cs₂CO₃ (1.45 g, 4.44 mmol) in Tol (10 mL) was stirred at 110° C. overnight under N₂. The reaction mixture was quenched with water and extracted with EtOAc (50 mL), the combined organic layer was washed with brine, dried over anhydrous Na₂SO₄ and evaporated under reduced pressure. The crude was purified by silica gel chromatography (25% EA/PE) to afford 499-2 (480 mg, yield: 73%) as yellow oil.

### The Synthesis of (1-(2-methylthiazol-5-yl)pyrrolidin-3-yl)methanamine (499-3)

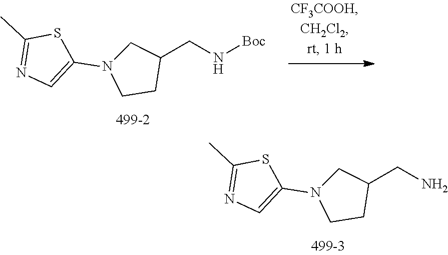 [SMILES: Cc1ncc(N2CCC(CNC(=O)OC(C)(C)C)C2)s1 ; Cc1ncc(N2CCC(CN)C2)s1]

To a stirred solution of 499-2 (297 mg, 1.00 mmol) in CH₂Cl₂ (10 mL) was added TFA (5 mL). The reaction mixture was stirred at room temperature for 1 hour until the reaction was complete (by LCMS). The mixture was concentrated under reduced pressure to afford 499-3 (195 mg, crude) as yellow oil, which was used to the next step without purification.

### The Synthesis of 4-(4-((4-(methylsulfonyl)benzyl)oxy)phenyl)-N-((1-(2-methylthiazol-5-yl)pyrrolidin-3-yl)methyl)-1H-imidazole-1-carboxamide (AC2020601-0499)

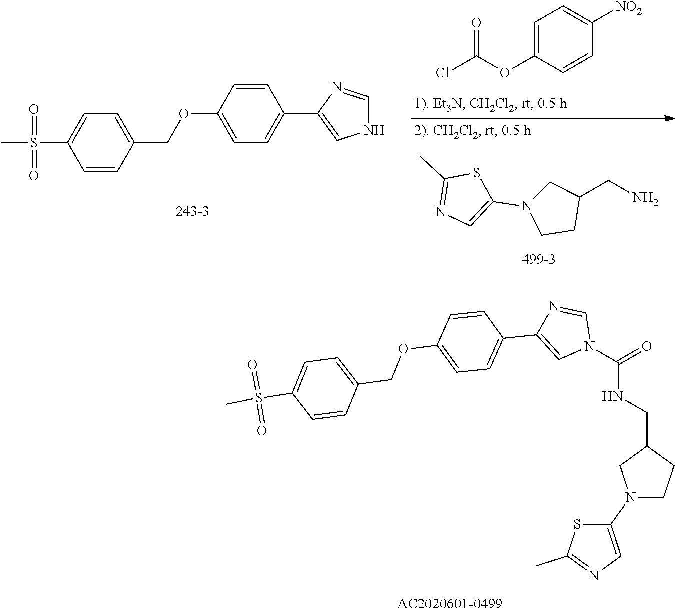 [SMILES: O=C(Cl)Oc1ccc([N+](=O)[O-])cc1 ; CS(=O)(=O)c1ccc(COc2ccc(-c3c[nH]cn3)cc2)cc1 ; Cc1ncc(N2CCC(CN)C2)s1 ; Cc1ncc(N2CCC(CNC(=O)n3cnc(-c4ccc(OCc5ccc(S(C)(=O)=O)cc5)cc4)c3)C2)s1]

To a solution of 243-3 (150 mg, 0.46 mmol) in DCM (5 mL) was added 4-nitrophenyl carbonochloridate (101 mg, 0.50 mmol) and Et₃N (2 mL) at 0° C. and stirred for 0.5 hour. Then the reaction mixture was allowed to warm to room temperature and 499-3 (91 mg, 0.46 mmol) was added. After 0.5 hour stirring, the reaction mixture was quenched with water, extracted with DCM (20 mL), the combined organic layer was washed with brine, dried over anhydrous Na₂SO₄ and evaporated under reduced pressure. The crude was purified by silica gel chromatography (5% MeOH/DCM) to afford AC2020601-0499 (15 mg, yield: 5.9%) as a white solid. ¹H NMR (400 MHz, DMSO-d₆) δ 8.63 (t, J=5.6 Hz, 1H), 8.29 (d, J=1.2 Hz, 1H), 8.02 (d, J=1.2 Hz, 1H), 7.96 (d, J=8.4 Hz, 2H), 7.07 (d, J=8.8 Hz, 4H), 6.43 (s, 1H), 5.28 (s, 2H), 3.24-3.37 (m, 4H), 3.22 (s, 3H), 3.14-3.19 (m, 1H), 3.00-3.03 (m, 1H), 2.62-2.67 (m, 1H), 2.45 (s, 3H), 2.08-2.14 (m, 1H), 1.77-1.82 (m, 1H). LC-MS m/z: 552.1 [M+H]⁺. HPLC Purity (254 nm):82.03%; t_{R}=3.199 min.

AC2020601-0501

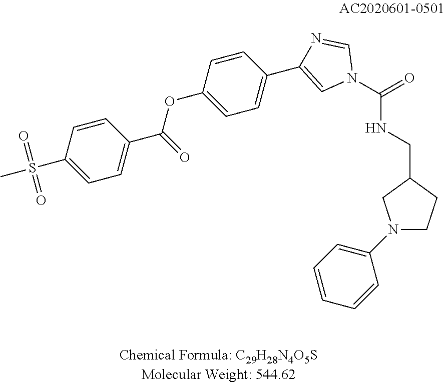 [SMILES: CS(=O)(=O)c1ccc(C(=O)Oc2ccc(-c3cn(C(=O)NCC4CCN(c5ccccc5)C4)cn3)cc2)cc1]

### The Synthesis of 4-(methylsulfonyl)benzoyl chloride (501-2)

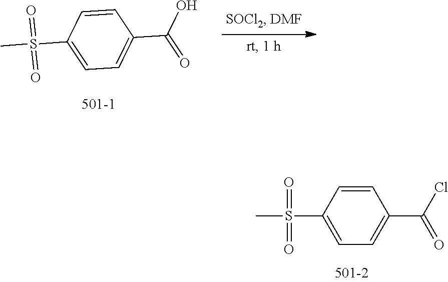

To a suspension of 501-1 (1.0 g, 5.0 mmol) in DMF (10 mL) was added SOCl₂ (892 mg, 7.5 mmol). The reaction mixture was stirred at room temperature for 1 hour until the reaction was complete (by LCMS). Water was added and the reaction mixture was extracted with EtOAc (50 mL), the combined organic layer was washed with brine, dried over anhydrous Na₂SO₄ and evaporated under reduced pressure. The crude was purified by silica gel chromatography (30% EA/PE) to afford 501-2 (700 mg, yield: 64%) as white oil.

### The Synthesis of tert-butyl 4-(4-(4-(methylsulfonyl)benzoyloxy)phenyl)-1H-imidazole-1-carboxylate (501-3)

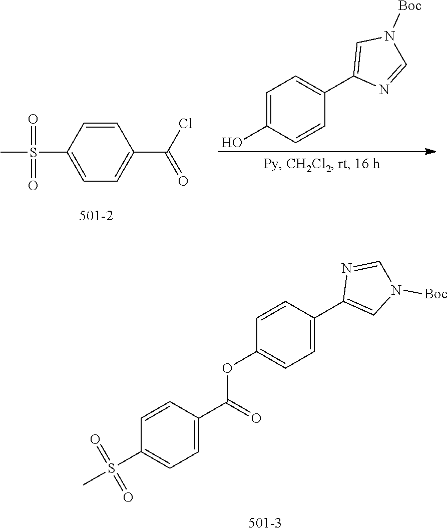 [SMILES: CS(=O)(=O)c1ccc(C(=O)Cl)cc1 ; CC(C)(C)OC(=O)n1cnc(-c2ccc(O)cc2)c1 ; CC(C)(C)OC(=O)n1cnc(-c2ccc(OC(=O)c3ccc(S(C)(=O)=O)cc3)cc2)c1]

To a suspension of 501-2 (700 mg, 3.2 mmol) in CH₂Cl₂ (15 mL) was added tert-butyl 4-(4-hydroxyphenyl)-1H-imidazole-1-carboxylate (416 mg, 1.6 mmol) and Py (10 mL). The reaction mixture was stirred at room temperature for 16 hours until the reaction was complete (by LCMS). Water was added and the reaction mixture was extracted with DCM (50 mL), the combined organic layer was washed with brine, dried over anhydrous Na₂SO₄ and evaporated under reduced pressure. The crude was purified by silica gel chromatography (30% EA/PE) to afford 501-3 (500 mg, yield: 35%) as yellow oil.

### The Synthesis of 4-(1H-imidazol-4-yl)phenyl 4-(methylsulfonyl)benzoate (501-4)

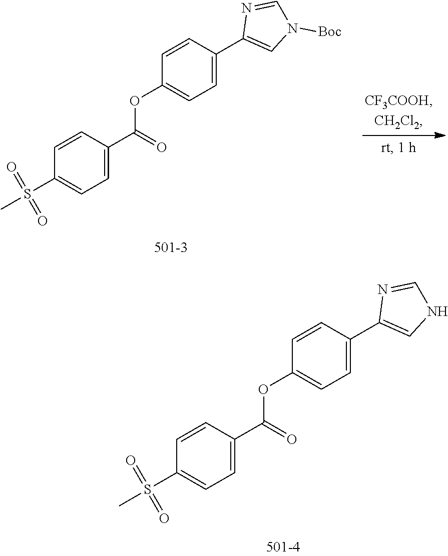 [SMILES: CC(C)(C)OC(=O)n1cnc(-c2ccc(OC(=O)c3ccc(S(C)(=O)=O)cc3)cc2)c1 ; CS(=O)(=O)c1ccc(C(=O)Oc2ccc(-c3c[nH]cn3)cc2)cc1]

To a stirred solution of 501-3 (500 mg, 1.13 mmol) in CH₂Cl₂ (10 mL) was added TFA (10 mL). The reaction mixture was stirred at room temperature for 1 hour until the reaction was complete (by LCMS). The reaction mixture was quenched with water, extracted with DCM (20 mL), the combined organic layer was washed with brine, dried over anhydrous Na₂SO₄. The mixture was concentrated under reduced pressure to afford 501-4 (300 mg, yield: 84%) as yellow oil, which was used to the next step without purification.

### The Synthesis of 4-(1-((1-phenylpyrrolidin-3-yl)methylcarbamoyl)-1H-imidazol-4-yl)phenyl 4-(methylsulfonyl)benzoate (AC2020601-0501)

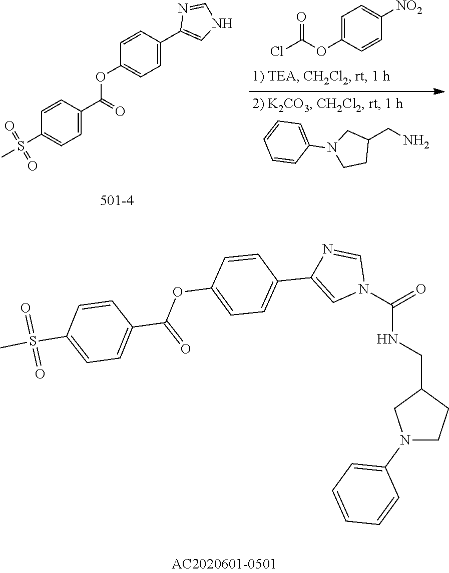 [SMILES: CS(=O)(=O)c1ccc(C(=O)Oc2ccc(-c3c[nH]cn3)cc2)cc1 ; NCC1CCN(c2ccccc2)C1 ; O=C(Cl)Oc1ccc([N+](=O)[O-])cc1 ; CS(=O)(=O)c1ccc(C(=O)Oc2ccc(-c3cn(C(=O)NCC4CCN(c5ccccc5)C4)cn3)cc2)cc1]

To a solution of 501-4 (300 mg, 0.9 mmol) in DCM (5 mL) was added 4-nitrophenyl carbonochloridate (271 mg, 1.35 mmol) and Et₃N (5 mL) at room temperature and stirred for 1 hour. Then (1-phenylpyrrolidin-3-yl)methanamine (316 mg, 1.8 mmol) and K₂CO₃ (248 mg, 1.8 mmol) was added. After 1 hour stirring, the reaction mixture was quenched with water, extracted with DCM (20 mL), the combined organic layer was washed with brine, dried over anhydrous Na₂SO₄ and evaporated under reduced pressure. The crude was purified by silica gel chromatography (5% MeOH/DCM) to afford AC2020601-0501 (9.09 mg, yield: 1.3%) as a yellow solid. ¹H NMR (400 MHz, DMSO-d₆) δ 8.73 (t, J=5.6 Hz, 1H), 8.37-8.39 (m, 3H), 8.16-8.20 (m, 3H), 7.92 (d, J=8.4 Hz, 2H), 7.38 (d, J=8.8 Hz, 2H), 7.16 (t, J=8.0 Hz, 2H), 6.59 (t, J=7.2 Hz, 1H), 6.54 (d, J=8.0 Hz, 2H), 3.36-3.42 (m, 7H), 3.24 (dd, J=16.0 Hz, J=7.2 Hz, 1H), 3.08 (dd, J=9.2 Hz, J=6.0 Hz, 1H), 2.62-2.68 (m, 1H), 2.12-2.16 (m, 1H), 1.80-1.85 (m, 1H). LC-MS m/z: 545.1 [M+H]⁺. HPLC Purity (254 nm): 94.03%; t_{R}=3.850 min.

AC2020601-0506

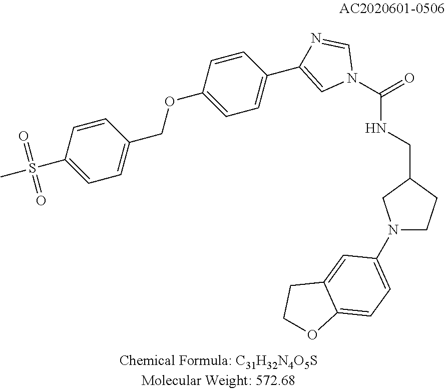 [SMILES: CS(=O)(=O)c1ccc(COc2ccc(-c3cn(C(=O)NCC4CCN(c5ccc6c(c5)CCO6)C4)cn3)cc2)cc1]

### The Synthesis of tert-butyl (1-(2,3-dihydrobenzofuran-5-yl)pyrrolidin-3-yl)methylcarbamate (506-2)

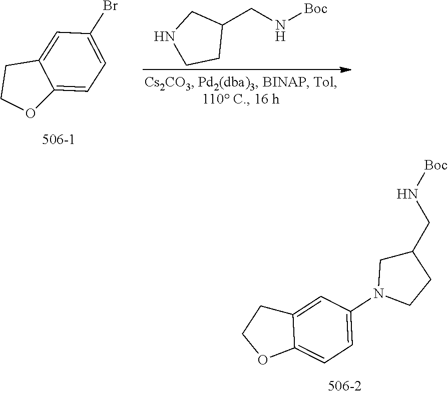 [SMILES: Brc1ccc2c(c1)CCO2 ; CC(C)(C)OC(=O)NCC1CCNC1 ; CC(C)(C)OC(=O)NCC1CCN(c2ccc3c(c2)CCO3)C1]

To a suspension of 506-1 (398 mg, 2.0 mmol) in Tol (10 mL) was added tert-butyl pyrrolidin-3-ylmethylcarbamate (200 mg, 1.0 mmol), Cs₂CO₃ (650 mg, 2.0 mmol), Pd₂(dba)₃ (57 mg, 0.1 mmol) and BINAP (6₂ mg, 0.1 mmol). The reaction mixture was allowed to warm to 100° C. for 16 hours until the reaction was complete (by LCMS). Water was added and the reaction mixture was extracted with EtOAc (50 mL), the combined organic layer was washed with brine, dried over anhydrous Na₂SO₄ and evaporated under reduced pressure. The crude was purified by silica gel chromatography (30% EA/PE) to afford 506-2 (300 mg, yield: 49.8%) as yellow oil.

### The Synthesis of (1-(2,3-dihydrobenzofuran-5-yl)pyrrolidin-3-yl)methanamine (506-3)

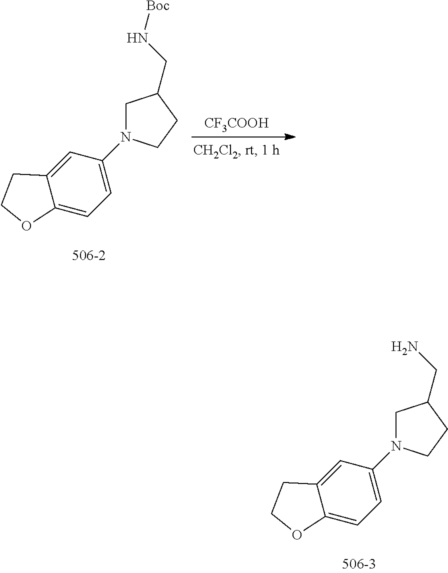 [SMILES: CC(C)(C)OC(=O)NCC1CCN(c2ccc3c(c2)CCO3)C1 ; "CF3COOH" ; NCC1CCN(c2ccc3c(c2)CCO3)C1]

To a stirred solution of 506-2 (300 mg, 0.94 mmol) in CH₂Cl₂ (10 mL) was added TFA (10 mL). The reaction mixture was stirred at room temperature for 1 hour until the reaction was complete (by LCMS). The reaction mixture was quenched with water, extracted with DCM (20 mL), the combined organic layer was washed with brine, dried over anhydrous Na₂SO₄. The mixture was concentrated under reduced pressure to afford 506-3 (150 mg, yield: 73.9%) as yellow oil, which was used to the next step without purification.

### The Synthesis of N-((1-(2,3-dihydrobenzofuran-5-yl)pyrrolidin-3-yl)methyl)-4-(4-(4-(methylsulfonyl)benzyloxy)phenyl)-1H-imidazole-1-carboxamide (AC2020601-0506)

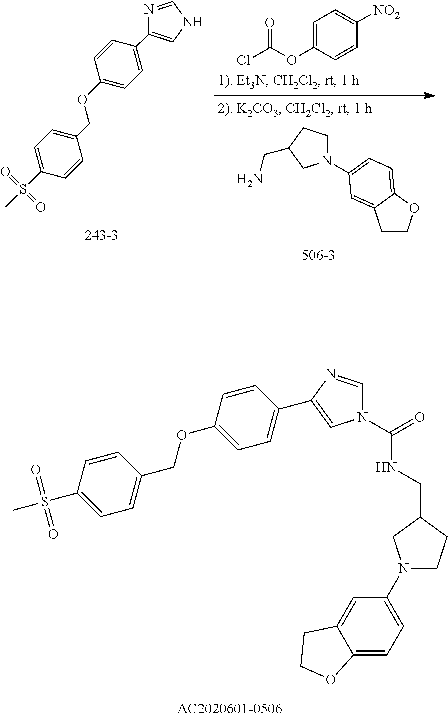 [SMILES: CS(=O)(=O)c1ccc(COc2ccc(-c3c[nH]cn3)cc2)cc1 ; O=C(Cl)Oc1ccc([N+](=O)[O-])cc1 ; NCC1CCN(c2ccc3c(c2)CCO3)C1 ; CS(=O)(=O)c1ccc(COc2ccc(-c3cn(C(=O)NCC4CCN(c5ccc6c(c5)CCO6)C4)cn3)cc2)cc1]

To a solution of 243-3 (150 mg, 0.38 mmol) in DCM (5 mL) was added 4-nitrophenyl carbonochloridate (114 mg, 0.57 mmol) and Et₃N (5 mL) at room temperature and stirred for 1 hour. Then 506-3 (150 mg, 0.76 mmol) and K₂CO₃ (105 mg, 0.76 mmol) was added. After 1 hour stirring, the reaction mixture was quenched with water, extracted with DCM (20 mL), the combined organic layer was washed with brine, dried over anhydrous Na₂SO₄ and evaporated under reduced pressure. The crude was purified by silica gel chromatography (5% MeOH/DCM) to afford AC2020601-0506 (6.14 mg, yield: 1.4%) as white solid. ¹H NMR (400 MHz, DMSO-d₆) δ 8.63 (t, J=5.2 Hz, 1H), 8.30 (s, 1H), 8.03 (s, 1H), 7.96 (d, J=8.4 Hz, 2H), 7.74 (t, J=8.4 Hz, 4H), 7.07 (d, J=8.8 Hz, 2H), 6.60 (d, J=8.4 Hz, 1H), 6.51 (s, 1H), 6.28 (d, J=8.4 Hz, 1H), 5.28 (s, 2H), 4.39 (t, J=8.8 Hz, 2H), 3.25-3.31 (m, 4H), 3.22 (s, 3H), 3.15-3.19 (m, 1H), 3.10 (t, J=8.8 Hz, 2H), 2.99-3.03 (m, 1H), 2.57-2.62 (m, 1H), 2.08-2.12 (m, 1H), 1.75-1.80 (m, 1H). LC-MS m/z: 573.2 [M+H]⁺. HPLC Purity (254 nm): 79.27%; t_{R}=1.856 min.

AC2020601-0507

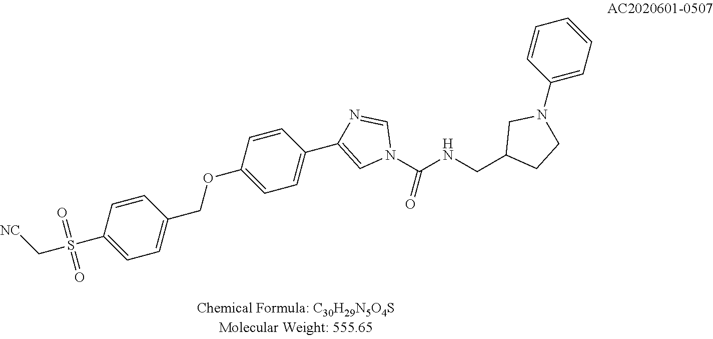 [SMILES: N#CCS(=O)(=O)c1ccc(COc2ccc(-c3cn(C(=O)NCC4CCN(c5ccccc5)C4)cn3)cc2)cc1]

### The Synthesis of methyl 4-(cyanomethylthio)benzoate (507-2)

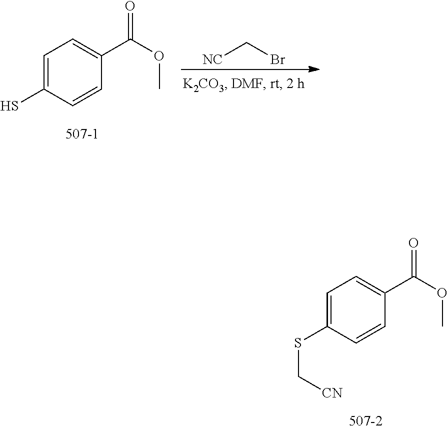

To a suspension of 507-1 (5.0 g, 30.0 mmol) in DMF (30 mL) was added 2-bromoacetonitrile (10.0 g, 60.0 mmol) and K₂CO₃ (8.3 g, 60.0 mmol). The reaction mixture was stirred at room temperature for 2 hours until the reaction was complete (by LCMS). Water was added and the reaction mixture was extracted with EtOAc (50 mL), the combined organic layer was washed with brine, dried over anhydrous Na₂SO₄ and evaporated under reduced pressure. The crude was purified by silica gel chromatography (15% PE/EA) to afford 507-2 (6.0 g, yield: 96.4%) as a white solid.

### The Synthesis of 2-(4-(hydroxymethyl)phenylthio)acetonitrile (507-3)

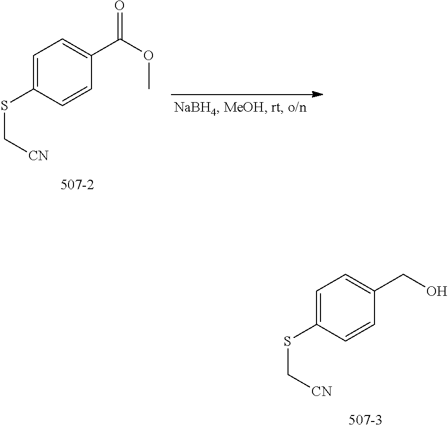

To a suspension of 507-2 (6.0 g, 29 mmol) in MeOH (20 mL) was added NaBH₄ (1.6 g, 43.5 mmol). The reaction mixture was stirred at room temperature for 2 hours until the reaction was complete (by LCMS). Water was added and the reaction mixture was extracted with EtOAc (50 mL), the combined organic layer was washed with brine, dried over anhydrous Na₂SO₄ and evaporated under reduced pressure. The crude was purified by silica gel chromatography (15% PE/EA) to afford 507-3 (3.0 g, yield: 41.5%) as white oil.

### The Synthesis of tert-butyl 4-(4-(4-(cyanomethylthio)benzyloxy)phenyl)-1H-imidazole-1-carboxylate (507-4)

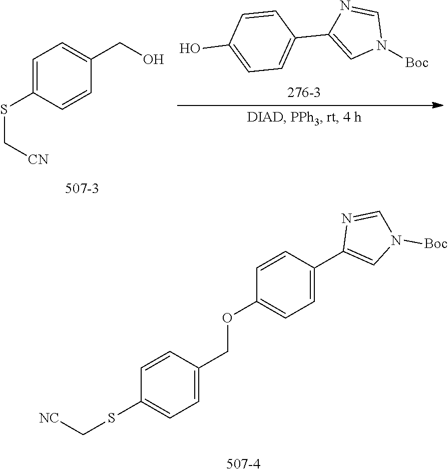 [SMILES: N#CCSc1ccc(CO)cc1 ; CC(C)(C)OC(=O)n1cnc(-c2ccc(O)cc2)c1 ; CC(C)(C)OC(=O)n1cnc(-c2ccc(OCc3ccc(SCC#N)cc3)cc2)c1]

To a suspension of 507-3 (3.0 g, 16.7 mmol) in THF (20 mL) was added 276-3 (2.17 g, 8.35 mmol), PPh₃ (2.62 g, 10.0 mmol) and DIAD (2.2 g, 10.9 mmol). The reaction mixture was stirred at room temperature for 4 hours until the reaction was complete (by LCMS). Water was added and the reaction mixture was extracted with EtOAc (50 mL), the combined organic layer was washed with brine, dried over anhydrous Na₂SO₄ and evaporated under reduced pressure. The crude was purified by silica gel chromatography (45% EA/PE) to afford 507-4 (800 mg, yield: 11.4%) as yellow oil.

### The Synthesis of tert-butyl 4-(4-(4-(cyanomethylsulfonyl)benzyloxy)phenyl)-1H-imidazole-1-carboxylate (507-5)

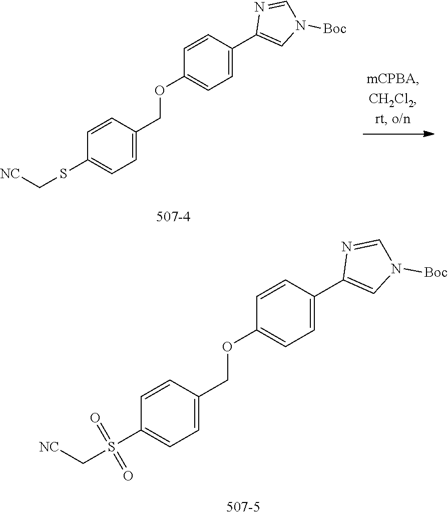 [SMILES: CC(C)(C)OC(=O)n1cnc(-c2ccc(OCc3ccc(SCC#N)cc3)cc2)c1 ; CC(C)(C)OC(=O)n1cnc(-c2ccc(OCc3ccc(S(=O)(=O)CC#N)cc3)cc2)c1]

To a stirred solution of 507-4 (800 mg, 1.9 mmol) in CH₂Cl₂ (10 mL) was added mCPBA (655 mg, 3.8 mmol). The reaction mixture was stirred at room temperature for 16 hour until the reaction was complete (by LCMS). The reaction mixture was quenched with water, extracted with DCM (20 mL), the combined organic layer was washed with brine, dried over anhydrous Na₂SO₄. The mixture was concentrated under reduced pressure to afford 507-5 (300 mg, yield: 47.5%) as white oil.

### The Synthesis of 2-(4-((4-(1H-imidazol-4-yl)phenoxy)methyl)phenylsulfonyl)acetonitrile (507-6)

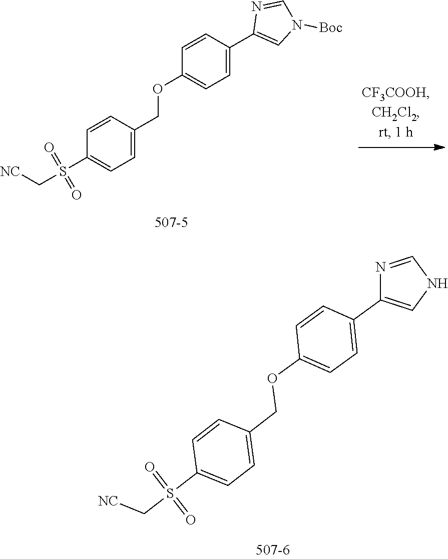 [SMILES: CC(C)(C)OC(=O)n1cnc(-c2ccc(OCc3ccc(S(=O)(=O)CC#N)cc3)cc2)c1 ; N#CCS(=O)(=O)c1ccc(COc2ccc(-c3c[nH]cn3)cc2)cc1]

To a stirred solution of 507-5 (300 mg, 0.66 mmol) in CH₂Cl₂ (10 mL) was added TFA (10 mL). The reaction mixture was stirred at room temperature for 1 hour until the reaction was complete (by LCMS). The reaction mixture was quenched with water, extracted with DCM (20 mL), the combined organic layer was washed with brine, dried over anhydrous Na₂SO₄. The mixture was concentrated under reduced pressure to afford 507-6 (100 mg, yield: 42.6%) as yellow oil, which was used to the next step without purification.

### The Synthesis of 4-(4-(4-(cyanomethylsulfonyl)benzyloxy)phenyl)-N-((1-phenylpyrrolidin-3-yl)methyl)-1H-imidazole-1-carboxamide (AC2020601-0507)

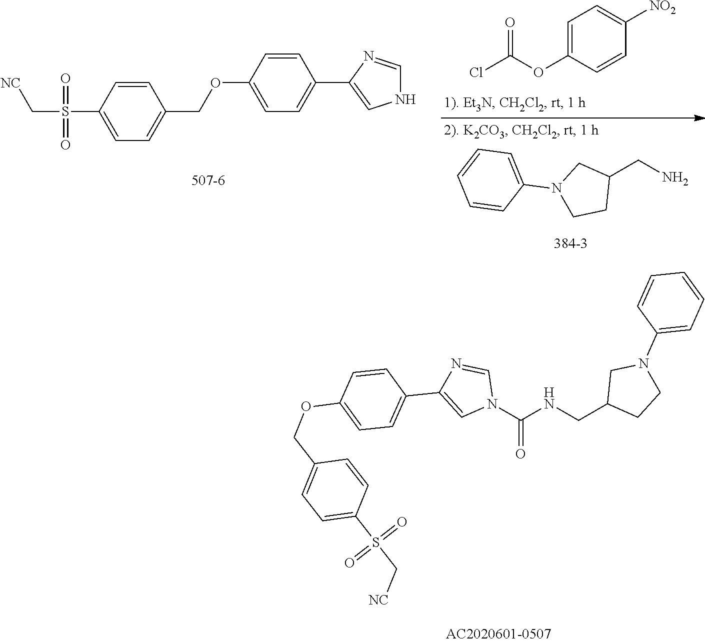 [SMILES: N#CCS(=O)(=O)c1ccc(COc2ccc(-c3c[nH]cn3)cc2)cc1 ; O=C(Cl)Oc1ccc([N+](=O)[O-])cc1 ; NCC1CCN(c2ccccc2)C1 ; N#CCS(=O)(=O)c1ccc(COc2ccc(-c3cn(C(=O)NCC4CCN(c5ccccc5)C4)cn3)cc2)cc1]

To a solution of 507-6 (100 mg, 0.28 mmol) in DCM (5 mL) was added 4-nitrophenyl carbonochloridate (84 mg, 0.42 mmol) and Et₃N (5 mL) at room temperature and stirred for 1 hour. Then (1-phenylpyrrolidin-3-yl)methanamine (384-3, 99 mg, 0.56 mmol) and K₂CO₃ (77 mg, 0.56 mmol)was added. After 1 hour stirring, the reaction mixture was quenched with water, extracted with DCM (20 mL), the combined organic layer was washed with brine, dried over anhydrous Na₂SO₄ and evaporated under reduced pressure. The crude was purified by silica gel chromatography (5% MeOH/DCM) to afford AC2020601-0507 (4.3 mg, yield: 1.6%) as a white solid. ¹H NMR (400 MHz, DMSO-d₆) δ 8.65 (t, J=4.8 Hz, 1H), 8.30 (s, 1H), 8.00-8.04 (m, 3H), 7.82 (d, J=8.0 Hz, 2H), 7.75 (d, J=8.4 Hz, 2H), 7.15 (t, J=7.6 Hz, 2H), 7.08 (d, J=8.8 Hz, 2H), 6.58 (t, J=6.8 Hz, 1H), 6.53 (d, J=7.6 Hz, 2H), 5.32 (s, 2H), 5.27 (s, 2H), 3.38-3.40 (m, 4H), 3.23-3.27 (m, 1H), 3.05-3.09 (m, 1H), 2.59-2.67 (m, 1H), 2.10-2.15 (m, 1H), 1.78-1.83 (m, 1H). LC-MS m/z: 556.3 [M+H]⁺. HPLC Purity (254 nm): 95.40%; t_{R}=2.298 min.

### Example 3: Biological Data

TABLE 1
Acid ceramidase assay data of example compounds

| | AC in vitro IC₅₀ |
|---|---|
| | A: below 300 nM |
| | B: 301-3000 nM |
| Compound ID | C: above 3000 nM |
| AC2020601-0167 | A |
| AC2020601-0199 | A |
| AC2020601-0205 | A |
| AC2020601-0206 | A |
| AC2020601-0243 | A |
| AC2020601-0245 | A |
| AC2020601-0246 | A |
| AC2020601-0275 | A |
| AC2020601-0276 | A |
| AC2020601-0278 | C |
| AC2020601-0284 | A |
| AC2020601-0294 | A |
| AC2020601-0295 | A |
| AC2020601-0296 | A |
| AC2020601-0303 | B |
| AC2020601-0306 | A |
| AC2020601-0321 | A |
| AC2020601-0323 | A |
| AC2020601-0324 | A |
| AC2020601-0327 | A |
| AC2020601-0328 | A |
| AC2020601-0339 | A |
| AC2020601-0342 | A |
| AC2020601-0347 | A |
| AC2020601-0348 | A |
| AC2020601-0349 | A |
| AC2020601-0363 | A |
| AC2020601-0373 | A |
| AC2020601-0374 | A |
| AC2020601-0377 | C |
| AC2020601-0379 | C |
| AC2020601-0380 | C |
| AC2020601-0382 | A |
| AC2020601-0384a | A |
| AC2020601-0384b | A |
| AC2020601-0401 | A |
| AC2020601-0402 | C |
| AC2020601-0407 | A |
| AC2020601-0428 | A |
| AC2020601-0430 | A |
| AC2020601-0431 | A |
| AC2020601-0434 | A |
| AC2020601-0437 | A |
| AC2020601-0438 | A |
| AC2020601-0439 | A |
| AC2020601-0443 | A |
| AC2020601-0444 | A |
| AC2020601-0445 | A |
| AC2020601-0447 | A |
| AC2020601-0448 | A |
| AC2020601-0449 | A |
| AC2020601-0451 | A |
| AC2020601-0452 | A |
| AC2020601-0456 | A |
| AC2020601-0472 | A |
| AC2020601-0477 | A |
| AC2020601-0478 | A |
| AC2020601-0481 | A |
| AC2020601-0482 | A |
| AC2020601-0488 | A |
| AC2020601-0489 | A |
| AC2020601-0491 | A |
| AC2020601-0494 | A |
| AC2020601-0496 | A |
| AC2020601-0497 | A |
| AC2020601-0498 | A |
| AC2020601-0499 | A |
| AC2020601-0501 | A |
| AC2020601-0506 | A |
| AC2020601-0507 | A |

The invention claimed is:

1. A compound, or a pharmaceutically acceptable salt thereof, having the formula: 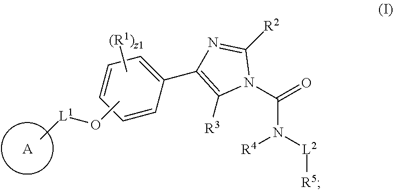
wherein
L¹ is a bond, —C(O)—, substituted or unsubstituted alkylene, or substituted or unsubstituted heteroalkylene;
Ring A is substituted or unsubstituted cycloalkyl, substituted or unsubstituted heterocycloalkyl, substituted or unsubstituted aryl, or substituted or unsubstituted heteroaryl;
R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)OR^{1C}, —C(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R¹ substituents may optionally be joined to form a substituted or unsubstituted cycloalkyl, substituted or unsubstituted heterocycloalkyl, substituted or unsubstituted aryl, or substituted or unsubstituted heteroaryl;
z1 is an integer from 0 to 4;
19 R² is hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR²D, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is hydrogen, halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NR^{3C}NR^{3A}R^{3B}, —ONR^{3A}R^{3B}, —NHC(O)NR^{3C}NR^{3A}R^{3B}, —NHC(O)NR^{3A}R^{3B}, —N(O)ₘ₃, —NR^{3A}R^{3B}, —C(O)R^{3C}, —C(O)OR^{3C}, —C(O)NR^{3A}R^{3B}, —OR^{3D}, —SR^{3D}, —NR^{3A}SO₂R^{3D}, —NR^{3A}C(O)R^{3C}, —NR^{3A}C(O)OR^{3C}, —NR^{3A}OR^{3C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁴ is hydrogen or unsubstituted C₁-C₄ alkyl;
L² is -L^{2A}-L^{2B}-L^{2C};
L^{2A}, L^{2B}, and L^{2C} are independently a bond, —O—, —NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R⁵ is hydrogen, halogen, —CX⁵₃, —CHX⁵₂, —CH₂X⁵, —OCX⁵₃, —OCH₂X⁵, —OCHX⁵₂, —CN, —SOₙ₅R^{5D}, —SOᵥ₅NR^{5A}R^{5B}, —NR^{5C}NR^{5A}R^{5B}, —ONR^{5A}R^{5B}, —NHC(O)NR^{5C}NR^{5A}R^{5B}, —NHC(O)NR^{5A}R^{5B}, —N(O)ₘ₅, —NR^{5A}R^{5B}, —C(O)R^{5C}, —C(O)OR^{5C}, —C(O)NR^{5A}R^{5B}, —OR^{5D}, —SR^{5D}, —NR^{5A}SO₂R^{5D}, —NR^{5A}C(O)R^{5C}, —NR^{5A}C(O)OR^{5C}, —NR^{5A}OR^{5C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{1A}, R^{1B}, R^{1C}, R^{1D}, R^{2A}, R^{2B}, R^{2C}, R^{2D}, R^{3A}, R^{3B}, R^{3C}, R^{3D}, R^{5A}, R^{5B}, R^{5C}, and R^{5D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{5A} and R^{5B} substituents bonded to the same nitrogen atom may optionally be joined to form a substituted or unsubstituted heterocycloalkyl or substituted or unsubstituted heteroaryl;
X¹, X², X³, and X⁵ are independently —F, —Cl, —Br, or —I;
n1, n2, n3, and n5 are independently an integer from 0 to 4; and
m1, m2, m3, m5, v1, v2, v3, and v5 are independently 1 or 2.

2. The compound of claim 1, having the formula:

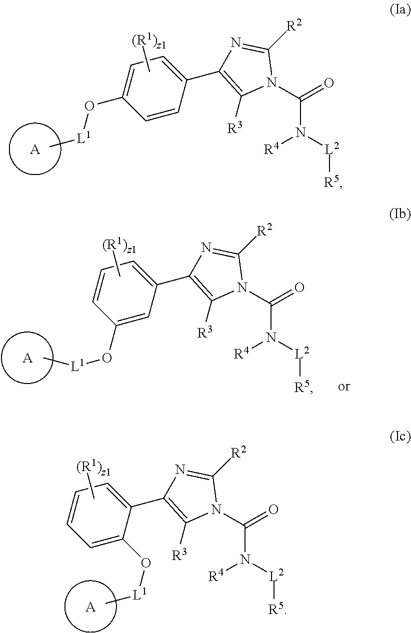

3. (canceled)

4. (canceled)

5. The compound of claim 1, having the formula: 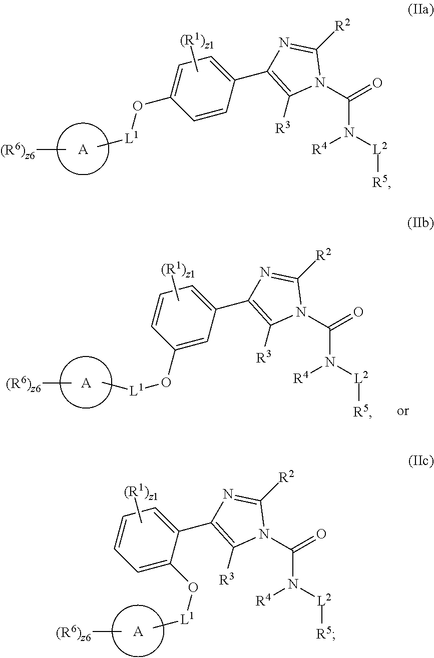
wherein
Ring A is cycloalkyl, heterocycloalkyl, aryl, or heteroaryl;
6 R⁶ is independently oxo, halogen, —CX⁶₃, —CHX⁶₂, —CH₂X⁶, —OCX⁶₃, —OCH₂X⁶, —OCHX⁶₂, —CN, —SOₙ₆R^{6D}, —S(O)(NH)R^{6D}, —SOᵥ₆NR^{6A}R^{6B}, —NR^{6C}NR^{6A}R^{6B}, —ONR^{6A}R^{6B}, —NHC(O)NR^{6C}NR^{6A}R^{6B}, —NHC(O)NR^{6A}R^{6B}, —N(O)ₘ₆, —NR^{6A}R^{6B}, —C(O)R^{6C}, —C(O)OR^{6C}, —C(O)NR^{6A}R^{6B}, —OR^{6D}, —SR^{6D}, —NR^{6A}SO₂R^{6D}, —NR^{6A}C(O)R^{6C}, —NR^{6A}C(O)OR^{6C}, —NR^{6A}OR⁶, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R⁶ substituents may optionally be joined to form a substituted or unsubstituted cycloalkyl, substituted or unsubstituted heterocycloalkyl, substituted or unsubstituted aryl, or substituted or unsubstituted heteroaryl;
R^{6A}, R^{6B}, R^{6C}, and R^{6D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{6A} and R^{6B} substituents bonded to the same nitrogen atom may optionally be joined to form a substituted or unsubstituted heterocycloalkyl or substituted or unsubstituted heteroaryl;
X⁶ is independently —F, —Cl, —Br, or —I;
n6 is independently an integer from 0 to 4;
m6 and v6 are independently 1 or 2; and
z6 is an integer from 0 to 11.

6. (canceled)

7. The compound of claim 5, wherein

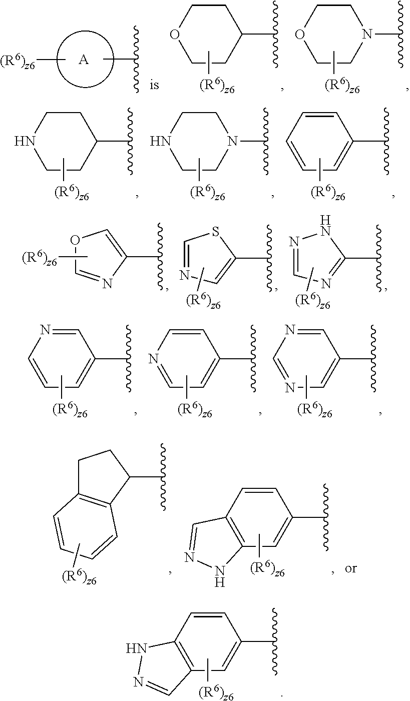

8. The compound of claim 5, wherein R⁶ is independently halogen, —CF₃, —CN, —SOₙ₆R^{6D}, —S(O)(NH)R^{6D}, —SOᵥ₆N^{6A}R^{6B}, —NR^{6A}R^{6B}, —C(O)NR^{6A}R^{6B}, —OR^{6D}, —SR^{6D}, —NR^{6A}SO₂R^{6D}, —NR^{6A}C(O)R^{6C}, substituted or unsubstituted C₁-C₆ alkyl, substituted or unsubstituted 2 to 6 membered heteroalkyl, or substituted or unsubstituted 3 to 6 membered heterocycloalkyl;
R^{6A} and R^{6B} are independently hydrogen, substituted or unsubstituted C₁-C₄ alkyl, substituted or unsubstituted 2 to 6 membered heteroalkyl, or substituted or unsubstituted 3 to 6 membered heterocycloalkyl; or R^{6A} and R^{6B} substituents bonded to the same nitrogen atom are joined to form a substituted or unsubstituted 3 to 6 membered heterocycloalkyl;
R^{6C} is independently unsubstituted C₃-C₆ cycloalkyl or substituted or unsubstituted 3 to 6 membered heterocycloalkyl; and
R^{6D} is independently hydrogen, —CHF₂, substituted or unsubstituted C₁-C₄ alkyl, unsubstituted 2 to 6 membered heteroalkyl, or substituted or unsubstituted 3 to 6 membered heterocycloalkyl.

9-12. (canceled)

13. The compound of claim 5, wherein R⁶ is independently —F, —CF₃, —CN, —SO₂CH₃, —SO₂NH₂, —SO₂NHCH₃, —S(O)CH₃, —S(O)(NH)CH₃, —NH₂, —C(O)NH₂, —SCH₃, —OH, —NHSO₂CH₃, —NHSO₂CHF₂, —CH₃,

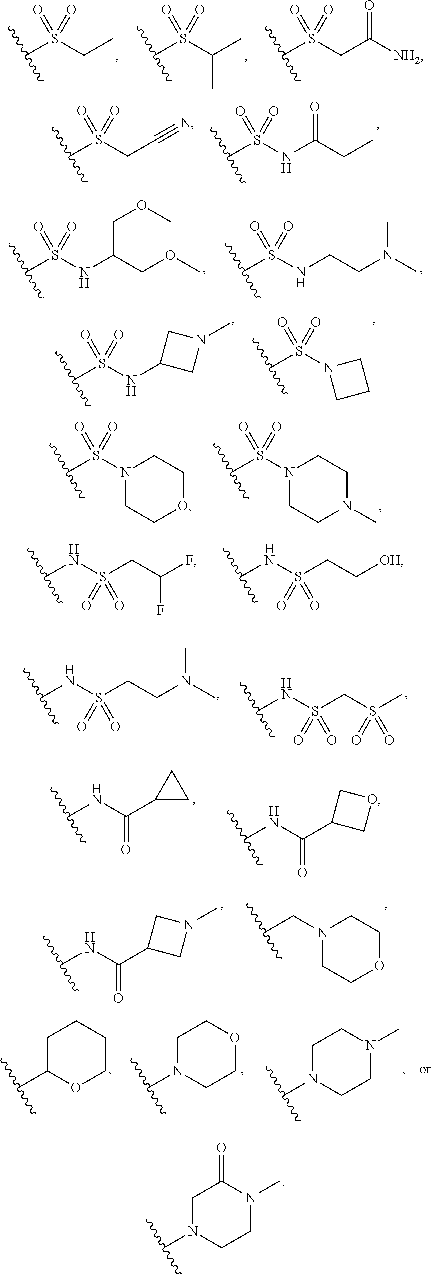

14. The compound of claim 5, wherein z6 is 0, 1, or 2.

15. (canceled)

16. (canceled)

17. The compound of claim 5, wherein

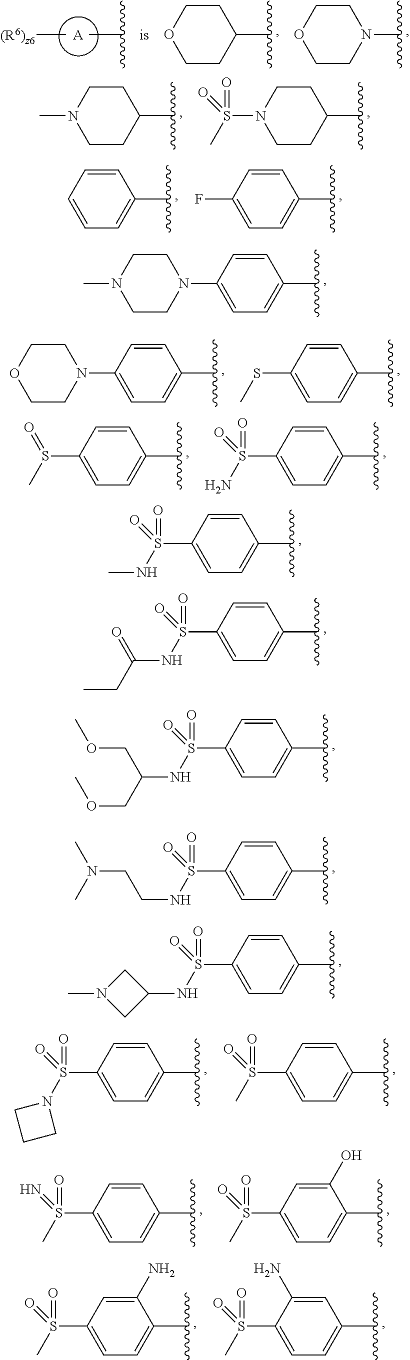

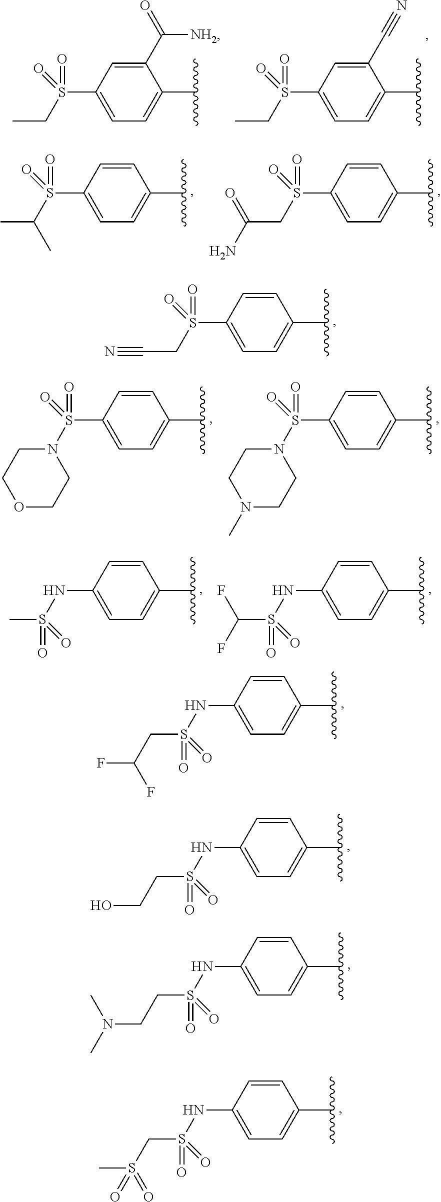

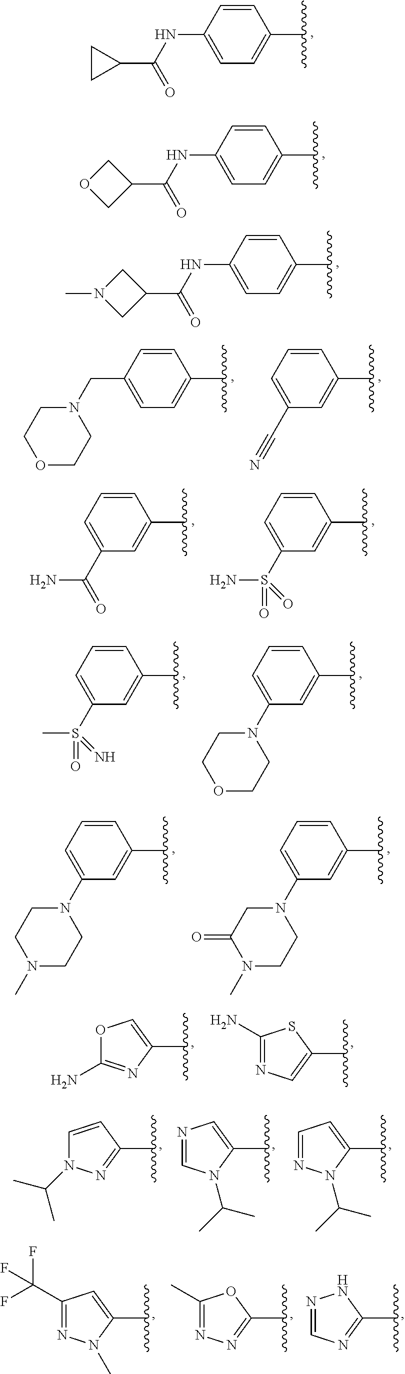

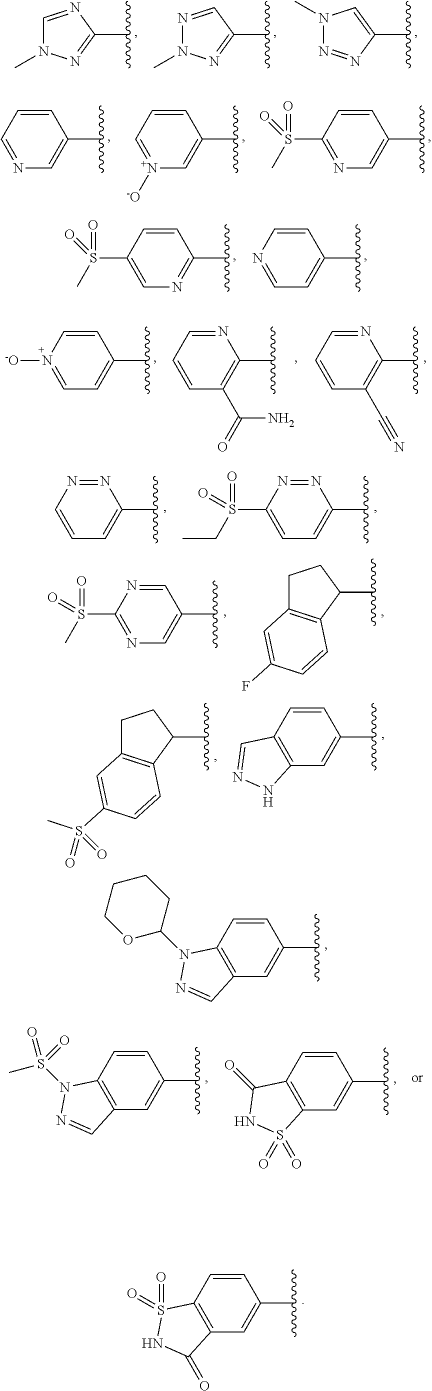

18. The compound of claim 1, wherein L¹ is a bond, —C(O)—, substituted or unsubstituted C₁-C₄ alkylene, or substituted or unsubstituted 2 to 4 membered heteroalkylene.

19. The compound of claim 1, wherein L¹ is a bond, —C(O)—,

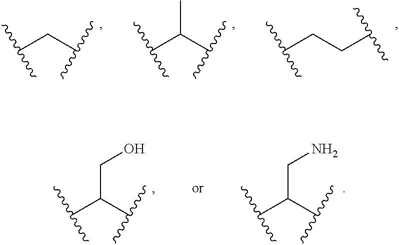

20-22. (canceled)

23. The compound of claim 1, wherein
R² is hydrogen or halogen;
R³ is hydrogen or halogen; and
R⁴ is hydrogen or unsubstituted methyl.

24-27. (canceled)

28. The compound of claim 1, wherein
L^{2A} is unsubstituted C₁-C₆ alkylene;
L^{2B} is a bond, —O—, —NH—, unsubstituted C₁-C₆ alkylene, unsubstituted C₃-C₆ cycloalkylene, or unsubstituted 3 to 6 membered heterocycloalkylene; and
L^{2C} is a bond, —O—, —NH—, unsubstituted C₁-C₆ alkylene, unsubstituted phenylene, or unsubstituted 5 to 6 membered heteroarylene.

29. (canceled)

30. (canceled)

31. The compound of claim 1, wherein L² is

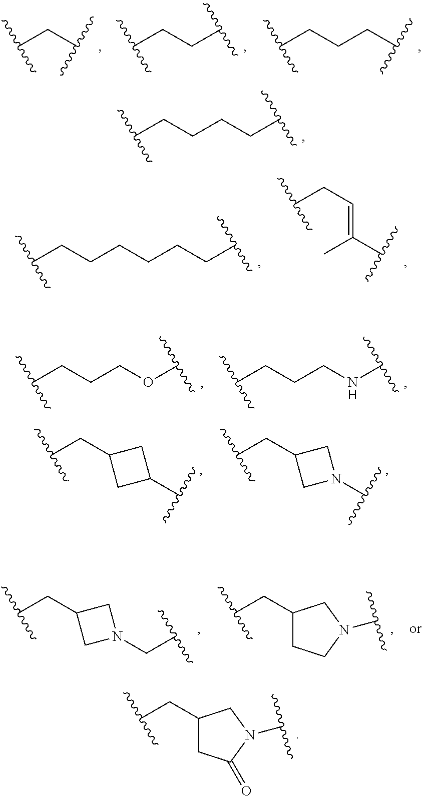

32. (canceled)

33. The compound of claim 1, wherein R⁵ is hydrogen, halogen, —CF₃, —CHF₂, —CH₂F, —OCF₃, —OCHF₂, —OCH₂F, —CN, —SF₅, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl.

34. The compound of claim 1, wherein R⁵ is hydrogen, —CF₃, —CHF₂, —OCF₃

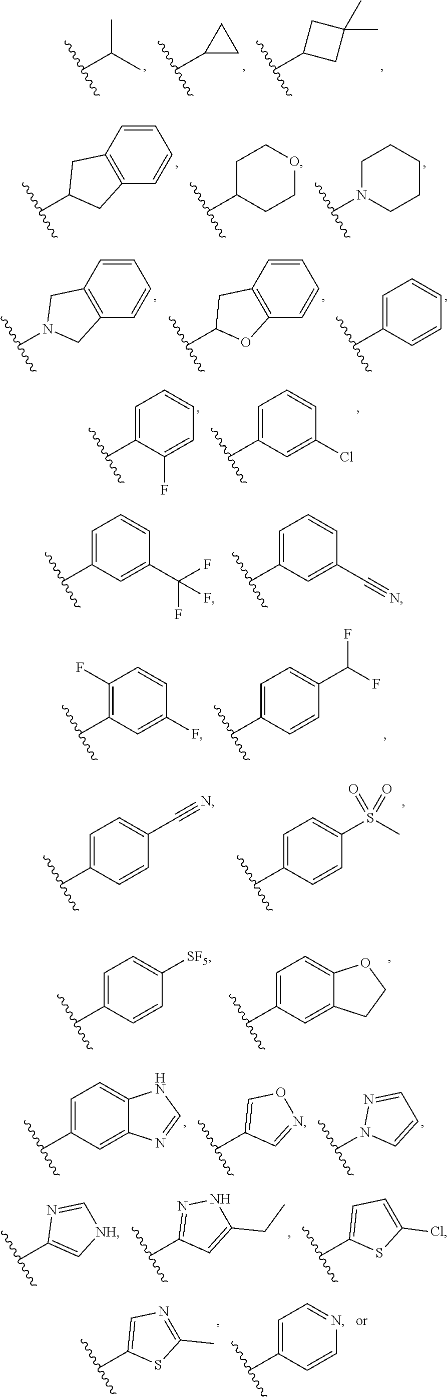

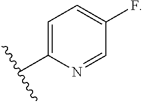 [SMILES: *c1ccc(F)cn1]

35. The compound of claim 1, wherein -L²-R⁵ is

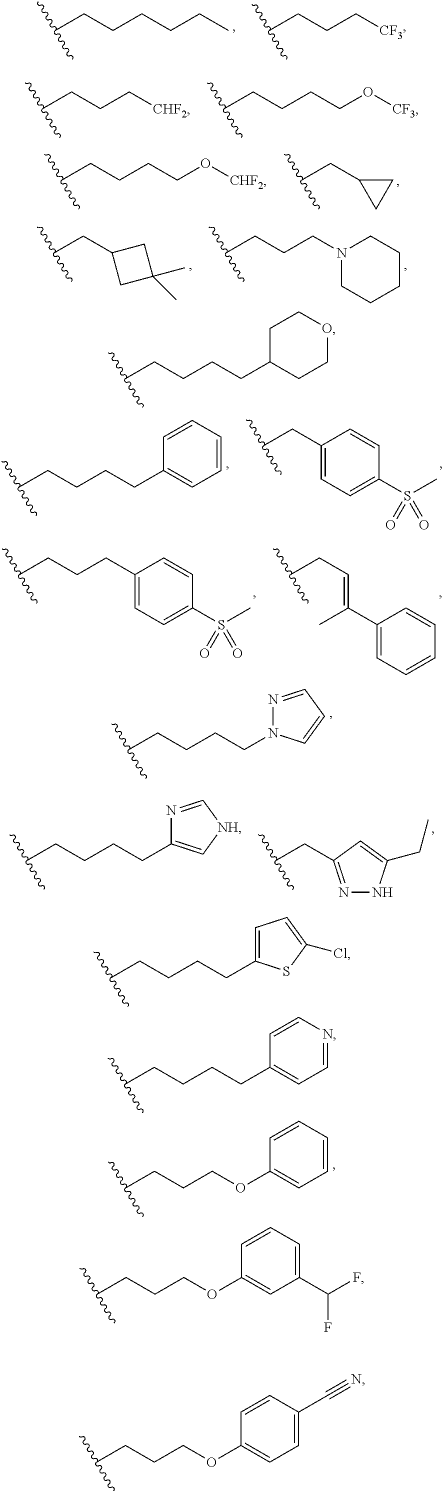

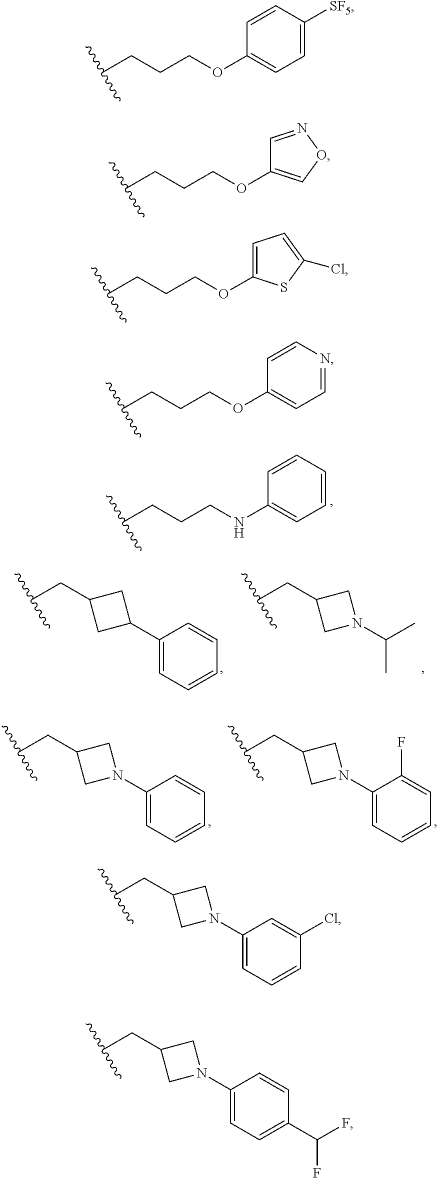

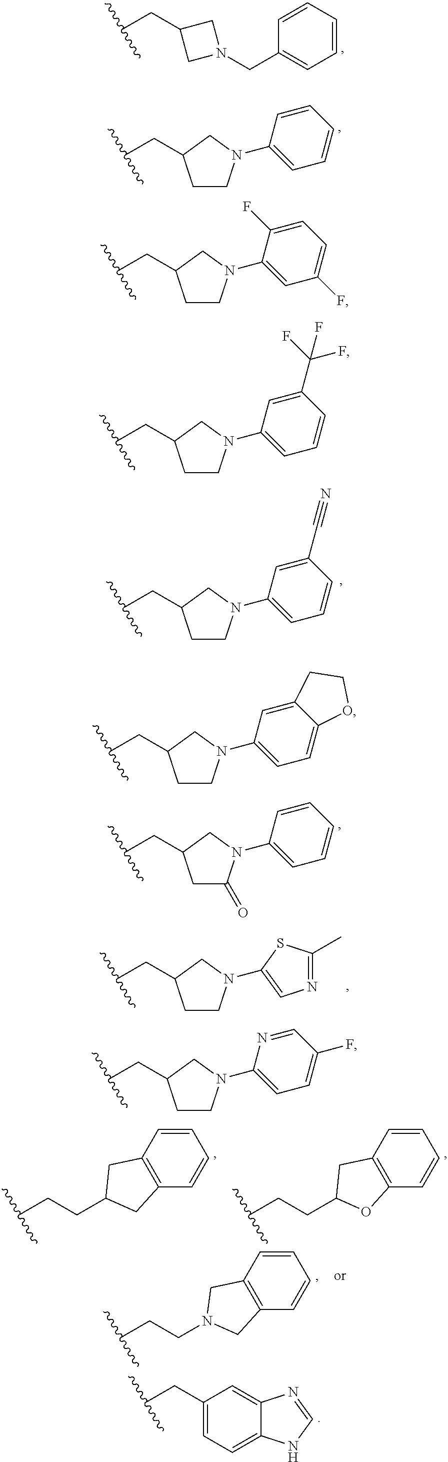

36. A pharmaceutical composition comprising the compound of claim 1, or a pharmaceutically acceptable salt thereof, and a pharmaceutically acceptable excipient.

37. A method of treating a fibrotic disease in a subject in need thereof, said method comprising administering to the subject in need thereof a therapeutically effective amount of an acid ceramidase inhibitor, or a pharmaceutically acceptable salt thereof.

38. A method of treating a fibrotic disease in a subject in need thereof, said method comprising administering to the subject in need thereof a therapeutically effective amount of a compound of claim 1, or a pharmaceutically acceptable salt thereof.

39. (canceled)

40. A method of treating cancer in a subject in need thereof, said method comprising administering to the subject in need thereof a therapeutically effective amount of a compound of claim 1, or a pharmaceutically acceptable salt thereof.

41. (canceled)
